(19) United States
(12) Patent Application Publication
(10) Pub. No.: US 2026/0027154 A1
(43) Pub. Date: Jan. 29, 2026

(54) Compositions And Methods For Enhancing Adoptive T Cell Therapeutics

(71) Applicants: The Regents of the University of California, Oakland, CA (US); Northwestern University, Evanston, IL (US)

(72) Inventors: Kole ROYBAL, San Francisco, CA (US); Julie GARCIA, San Francisco, CA (US); Iowis ZHU, San Francisco, CA (US); Jaehyuk CHOI, Chicago, IL (US); Jay DANIELS, Chicago, IL (US)

(21) Appl. No.: 19/179,303

(22) Filed: Apr. 15, 2025

Related U.S. Application Data
(62) Division of application No. 18/630,828, filed on Apr. 9, 2024, now Pat. No. 12,295,971.
Application No. 18/630,828 is a continuation of application No. PCT/US23/75738, filed on Oct. 2, 2023.
(60) Provisional application No. 63/412,300, filed on Sep. 30, 2022.

(51) Int. Cl. A61K 35/17; A61K 40/11; A61K 40/31; A61K 40/32; A61K 40/42; A61P 35/00; C07K 14/47; C07K 14/82; C07K 16/28; C12N 5/0783; C12N 9/16; C12N 9/22; C12N 15/63

(52) U.S. Cl. CPC A61K 35/17; A61K 40/11; A61K 40/31; A61K 40/32; A61K 40/4211; A61K 40/424; A61K 40/4251; A61P 35/00; C07K 14/4702; C07K 14/4747; C07K 14/82; C07K 16/2803; C12N 5/0636; C12N 9/16; C12N 9/22; C12N 15/63; A61K 2239/13; C12N 2310/20; C12N 2510/00

(57) ABSTRACT

The present disclosure relates generally to compositions and methods for improving T cell therapy. In particular, the disclosure provides polypeptides and recombinant nucleic acid constructs and/or recombinant nucleic acids encoding polypeptides having mutations capable of altering T cell signaling, cytokine production, and/or in vivo persistence in tumors of therapeutic T cells comprising the mutation. The T cell signaling can be by NFAT, NF-κB and/or AP-1 pathways. The disclosure also provides vectors and cells including the polypeptides and/or recombinant nucleic acid constructs and/or recombinant nucleic acids of the disclosure as well as methods of preparing a T cell for use in cell therapy, and methods of identifying a mutation useful for improving T cell therapy.

### CROSS-REFERENCE TO RELATED APPLICATION

This application is a divisional of U.S. patent application Ser. No. 18/630,828, filed Apr. 9, 2024, which is a continuation, filed under 35 U.S.C. § 120, of PCT/US2023/075738 on Oct. 2, 2023, which claims the benefit of priority to U.S. Provisional Patent Application No. 63/412,300 filed Sep. 30, 2022, the entire contents of which are incorporated by reference herein and for all purposes, including any drawings.

### STATEMENT REGARDING FEDERALLY SPONSORED R&D

This invention was made with government support under grant no. OD025751 and 1DP2AI136599-01 awarded by The National Institutes of Health. The government has certain rights in the invention.

### STATEMENT REGARDING SEQUENCE LISTING

The Sequence Listing XML associated with this application is provided in XML file format and is hereby incorporated by reference into the specification. The name of the XML file containing the Sequence Listing XML is 048536-737001WO_ST26.xml. The XML file is 720,329 bytes, and was created on Oct. 2, 2023, and is being submitted electronically via USPTO Patent Center.

### FIELD

The present disclosure relates generally to compositions and methods for enhancing T cell therapeutics. The disclosure provides recombinant nucleic acid constructs and/or recombinant nucleic acids encoding polypeptides that promote T cell signaling, efficacy and/or in vivo persistence, cells that comprise such recombinant nucleic acids, methods for preparing T cells for use in cell therapies, as well as methods for identifying mutations useful for improving T cell therapy.

### BACKGROUND

Adoptive T cell therapies, including chimeric antigen receptor (CAR) T cells, have revolutionized cancer therapy. However, impressive responses are limited to a subset of patients with hematological cancers and have not been unlocked in patients with solid tumors, which represent 90% of adult cancers. In both treatment-resistant hematological and solid cancers, adoptive T cell therapy is limited by a complex combination of factors including fitness of engineered T cells in tumors, T cell exhaustion, poor in vivo persistence and immunosuppressive environmental factors. Despite significant recent advances, rational design has failed to overcome the problems associated with such factors.

Another approach to identify modifications that improve T cell function in vitro and in vivo, besides rational design, is unbiased screening. For example, the vast majority of screening efforts, have focused on genome-scale or genome-wide alterations which modify expression of endogenous wild-type genes via CRISPR-Cas9 or short hairpin RNA (shRNA) or cDNA overexpression.

Chimeric antigen receptors (CARs) are synthetic receptors that include an antigen specific extracellular single chain variable fragment (scFv) attached to a flexible linker (hinge) region, transmembrane domain, and intracellular signaling domains. The intracellular portion of the receptor consists of T cell signaling domains such as 41BB, CD28 and CD3zeta, designed to mimic T cell receptor (TCR) stimulation and the immunological synapse upon engagement with the antigen specified by the scFv. CAR constructs do not require antigen presentation by MHC molecules, and therefore have been used to effectively redirect a patient's own T cells against a tumor specific cell surface antigen. To date, five CD19 targeted CAR-T cell therapies have been approved by the FDA for use against hematological B cell cancers. While these therapies have proven highly effective in refractory B cell malignancies, CAR-T cell therapies have yet to provide robust, long-term efficacy against solid tumors. In the solid tumor setting, CAR-T cells can become exhausted and struggle to proliferate and perform effector function, ultimately resulting in the inability to control tumor growth or prevent relapse. Therefore, to create effective targeted cellular therapies against solid tumors the proliferative capacity, persistence and effector function of CAR-T cells needs to be improved.

An avenue under investigation is genetically modifying CAR-T cells to improve their functionality in solid tumors. A recent case study described a chronic lymphocytic leukemia (CLL) patient who experienced a delayed yet complete response after treatment with a CD19 CAR-T cell therapy. It was later discovered that, within a single T cell clone, the CD19 CAR cassette had integrated into the one allele of TET2, a known T cell lymphoma tumor suppressor, rendering it nonfunctional. Interestingly, the second TET2 allele of this patient was also mutated, resulting in a lack of function of TET2 in the CD19 CAR-T cells dosed to this patient. This single TET2 knockout CAR-T cell clone exhibited altered T cell differentiation and improved overall effector function. Ultimately, this clone expanded to become a majority of the CAR-T cell population, and mediated a complete response against the patient's relapsed CLL. In a second example, a similar complete response was mediated in a patient when the CD22 CAR cassette integrated into the T cell lymphoma tumor suppressor CBL. These case studies demonstrate that genetic knockout of T cell lymphoma tumor suppressors, such as TET2 and CBL, can have remarkable beneficial effects on CAR-T cell therapies. In preclinical studies, genome wide knockout assays have revealed genes, such as REGNASE, that upon knockout improve T cell fitness and anti-tumor efficacy in vivo. Additionally, other studies have found that the knockout of genes related to T cell exhaustion and memory formation, such as the NR4A family of genes, can result in improved and prolonged CAR-T cell response to tumors.

While these examples indicate that CAR-T cell functionality can be improved through genetic manipulation, particularly through manipulation of tumor suppressor genes, these studies are often extremely broad in their scope (examining the entire genome) and focus solely on the effect of constitutive genetic knockouts. Somatic single nucleotide variant (SSNV) mutations, translocations and gene deletions that naturally arise in cancers offer biologically rational candidates for genetic manipulation alongside CAR expression.

There remains a need in the art for alternative solutions to address the significant unmet need for effective adoptive T cell therapies and for enhancing engineered T cell fitness.

### SUMMARY

This section provides a general summary of the disclosure, and is not comprehensive of its full scope or all of its features.

The disclosure provides recombinant nucleic acid constructs and/or recombinant nucleic acids and methods for enhancing adoptive T cell therapies. The recombinant nucleic acid constructs and/or recombinant nucleic acids of the disclosure encode polypeptides with mutations wherein the mutations enhance the therapeutic efficacy of T cells by altering T cell signaling, decreasing T cell exhaustion and/or by enhancing in vivo persistence and fitness of engineered T cells.

In one aspect, the disclosure provides a polypeptide comprising: (a) a caspase-associated recruitment domain (CARD) containing protein or a functional fragment thereof; and

(b) a domain capable of binding to (i) a substrate localized to the intracellular side of the plasma membrane of a cell and/or (ii) a target polypeptide comprising a phosphorylated tyrosine. In some embodiments, the domain in b) is capable of binding to a substrate indirectly localized to the intracellular side of the plasma membrane through binding to another polypeptide or lipid that is directly localized to the intracellular side. In some embodiments, the CARD containing protein comprises or consists of a sequence having at least 75%, 80%, 85%, 90%, 95%, 96%, 97%, 98%, 99%, or 100% identity to any one of SEQ ID NO: 261-289. In some embodiments, the CARD containing protein is selected from CARD9, CARD10, CARD11, and CARD14. In some embodiments, the CARD containing protein comprises or consists of a sequence having at least 75%, 80%, 85%, 90%, 95%, 96%, 97%, 98%, 99%, or 100% identity to any one of SEQ ID NO: 261-264. In some embodiments, the functional fragment of the CARD containing protein is derived from CARD11 and comprises or consists of a sequence having at least 75%, 80%, 85%, 90%, 95%, 96%, 97%, 98%, 99%, or 100% identity to SEQ ID NO: 263.

In some embodiments, the function of the CARD containing protein or the functional fragment thereof is to bind to a CARD domain on BCL10. In some embodiments, the functional fragment thereof comprises at least 40, at least 50, at least 60, at least 70, at least 80, at least 90, at least 100, at least 150, at least 200, at least 300, at least 400, or at least 500 amino acids.

In some embodiments, the cell is a T cell, a macrophage, a monocyte, or a natural killer (NK) cell. In some embodiments, activation of the cell produces the substrate localized to the intracellular side of the plasma membrane. In some embodiments, the substrate localized to the intracellular side of the plasma membrane of a cell is a phosphoinositide. In some embodiments, the phosphoinositide is selected from phosphatidylinositol (3,4,5)-trisphosphate (PIP3), phosphatidylinositol 4,5-bisphosphate (PI(4,5)P2). In some embodiments, the polypeptide binds to the phosphoinositide with a Kd of less than 50 PM, 10 PM, 5 μM, 1 μM, 0.5 μM, 0.1 μM, 0.05 μM, or 0.01 μM, and wherein the Kd is analyzed using SPR.

In some embodiments, the target polypeptide comprises or consists of a sequence having at least 75%, 80%, 85%, 90%, 95%, 96%, 97%, 98%, 99%, or 100% identity to any one of SEQ ID NOs: 298-304. In some embodiments, the target polypeptide is derived from IGF-1R, CTLA-4, or CD28. In some embodiments, the phosphorylated tyrosine is located at the position corresponding to pY1221 of SEQ ID NO: 302. In some embodiments, the phosphorylated tyrosine is located at the position corresponding to pY1346 of SEQ ID NO: 301.

In some embodiments, the polypeptide binds to the target polypeptide with a Kd of less than 10 μM, 5 μM, 1 μM, 0.5 μM, 0.1 μM, 0.05 μM, or 0.01 μM, and wherein the Kd is analyzed by fluorescence polarization assay. In some embodiments, the polypeptide has a higher affinity for the target polypeptide comprising the phosphorylated tyrosine than a control polypeptide without phosphorylation at the corresponding tyrosine position. In some embodiments, the polypeptide has at least 2-fold, at least 5-fold, at least 10-fold, at least 50-fold, or at least 100-fold, higher affinity (lower Kd) for the target polypeptide comprising the phosphorylated tyrosine than a control polypeptide without phosphorylation at the corresponding tyrosine position.

In some embodiments, the domain in (b) is, or comprises, a SH3 domain. In some embodiments, the domain in (b) is, or comprises, a phosphotyrosine-binding (PTB) domain. In some embodiments, the domain in (b) is, or comprises, a pleckstrin homology (PH) domain. In some embodiments, the domain in (b) is, or comprises, a SH2 domain.

In one aspect, the disclosure provides a polypeptide comprising: (i) a caspase-associated recruitment domain (CARD) containing protein or a functional fragment thereof; and (ii) a SH2 domain. In some embodiments, the CARD containing protein is CARD11. In some embodiments, the SH2 domain is capable of binding to a polypeptide comprising a phosphorylated tyrosine. In some embodiments, the SH2 domain comprises or consists of a sequence having at least 75%, 80%, 85%, 90%, 95%, 96%, 97%, 98%, 99%, or 100% identity to any one of SEQ ID NOs: 305 and 307-437. In some embodiments, the SH2 domain comprises the motif of a conserved arginine residue in the FLVR motif.

In some embodiments, the SH2 domain comprises or consists of a sequence having at least 75%, 80%, 85%, 90%, 95%, 96%, 97%, 98%, 99%, or 100% identity to SEQ ID NO: 305. In some embodiments, the SH2 domain is an engineered SH2 domain with an enhanced affinity for phosphotyorsine. In some embodiments, the SH2 domain comprises or consists of a sequence having at least 75%, 80%, 85%, 90%, 95%, 96%, 97%, 98%, 99%, or 100% identity to any one of SEQ ID NOs: 313-15.

In one aspect, the disclosure provides a polypeptide comprising: (i) a CARD domain derived from CARD11 protein; and (ii) a second polypeptide portion derived from PIK3R3 protein. In some embodiments, the second polypeptide portion comprises or consists of a sequence having at least 75%, 80%, 85%, 90%, 95%, 96%, 97%, 98%, 99%, or 100% identity to any one of SEQ ID NO: 205, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 246, SEQ ID NO: 247, SEQ ID NO: 249, SEQ ID NO: 251, SEQ ID NO: 253, and SEQ ID NO: 255.

In some embodiments, the polypeptide does not comprise a Coiled-coil domain or a portion thereof. In some embodiments, the polypeptide comprises a Coiled-coil domain or a portion thereof. In some embodiments, the Coiled-coil domain comprises or consists of a sequence having at least 75%, 80%, 85%, 90%, 95%, 96%, 97%, 98%, 99%, or 100% identity to any one of SEQ ID NOs: 290-293. In some embodiments, the Coiled-coil domain comprises or consists of a sequence having at least 75%, 80%, 85%, 90%, 95%, 96%, 97%, 98%, 99%, or 100% identity to any one of SEQ ID NO: 290.

In some embodiments, the polypeptide comprises or consists of about 10, about 20, about 30, about 40, about 50, about 60, about 80, about 100, about 120, about 140, about 150, about 160, about 180, about 200, about 220, about 240, about 250, about 260, about 280, or about 300 amino acids of the N-terminal portion of the Coiled-coil domain. In some embodiments, the polypeptide comprises no more than 10, 20, 30, 40, 50, 60, 80, 100, 120, 140, 150, 160, 180, 200, 220, 240, 250, 260, 280, or 300 amino acids of the N-terminal portion of the Coiled-coil domain.

In some embodiments, the domain in (b), or the SH2 domain, or the second polypeptide portion, is located at the N-terminus or the CARD domain, between the CARD domain and the Coiled-coil domain, or at the C-terminus of the CARD domain and/or the Coiled-coil domain. In some embodiments, the CARD domain is derived from a CARD11 protein followed by the Coiled-coil domain derived from the CARD11 protein.

In some embodiments, the domain in (b), or the SH2 domain, or the second polypeptide portion, is located close to the C-terminus of the polypeptide, wherein the polypeptide has no more than 50, 40, 30, 20, 15, 10, or 5 amino acids at the C-terminus of the domain in (b), or the SH2 domain, or the second polypeptide portion.

In some embodiments, the polypeptide does not comprise an inhibitory domain (ID) or a portion thereof. In some embodiments, the polypeptide comprises an inhibitory domain (ID) or a portion thereof. In some embodiments, the inhibitory domain (ID) comprises or consists of a sequence having at least 75%, 80%, 85%, 90%, 95%, 96%, 97%, 98%, 99%, or 100% identity to SEQ ID NO: 294. In some embodiments, the inhibitory domain (ID) comprises or consists of a sequence having at least 75%, 80%, 85%, 90%, 95%, 96%, 97%, 98%, 99%, or 100% identity to SEQ ID NO: 295.

In some embodiments, the polypeptide comprises or consists of about 10, about 20, about 30, about 40, about 50, about 60, about 80, about 100, about 120, about 140, about 150, about 160, about 180, or about 200 amino acids of the N-terminal portion of the inhibitory domain (ID). In some embodiments, the second polypeptide portion comprises no more than 10, 20, 30, 40, 50, 60, 80, 100, 120, 140, 150, 160, 180, or 200 amino acids of the N-terminal portion of the inhibitory domain (ID).

In some embodiments, the polypeptide does not comprise a sequence that is at least 90%, 95%, 96%, 97%, 98%, 99%, or 100% identical to SEQ ID NO: 297. In some embodiments, the polypeptide comprises one or more mutations corresponding to S615F, D357N, Y361C, E634K, and/or S655C of SEQ ID NO: 26.

In some embodiments, expression of the polypeptide in a T cell promotes its in vivo accumulation in tumors. In some embodiments, the T cell expresses an engineered immune receptor that binds to a target on a tumor cell. In some embodiments, the T cell is selected from the group consisting of a regulatory (Treg), a gamma delta T cell, an invariant iNKT cell, a MAIT cell, a CAR T cell, a tumor-infiltrating lymphocyte, and an engineered T cell comprising a transcriptional receptor.

In one aspect, the disclosure provides a polypeptide, comprising a mutation capable of: (i) altering T cell signaling through NFAT, NF-κB and/or AP-1 pathways, (ii) altering cytokine production, (iii) altering JAK/STAT signaling in T cells, (iv) altering co-stimulatory molecule signaling in T cells, (v) altering RAS/MEK/ERK signaling in T cells, (vi) altering phospholipase gamma signaling, (vii) altering a transcription factor activity in T cells, and/or (viii) altering or enhancing in vivo persistence in tumors of T cells comprising the mutation.

In one aspect, the disclosure provides a recombinant nucleic acid encoding a polypeptide described herein. In some embodiments, the nucleic acid comprises a promoter. In some embodiments, the promoter is a constitutive promoter. In some embodiments, the constitutive promoter is CD4 promoter, CD8a promoter, CD8b promoter, TCRa promoter, TCRb promoter, CD3d promoter, CD3g promoter, CD3e promoter, or CD3z promoter. In some embodiments, the promoter is a minimal TATA promoter, a pGK, actin promoter, CD25 promoter, IL2 promoter, IL7 promoter, IL15 promoter, KLRG-1 promoter, HLA-DR promoter, CD38 promoter, CD69 promoter, Ki-67 promoter, CD11a promoter, CD58 promoter, CD99 promoter, CD62L promoter, CD103 promoter, CCR4 promoter, CCR5 promoter, CCR6 promoter, CCR9 promoter, CCR10 promoter, CXCR3 promoter, CXCR4 promoter, CLA promoter, Granzyme A promoter, Granzyme B promoter, Perforin promoter, CD57 promoter, CD161 promoter, IL-18Ra promoter, CD69 promoter, GzmB promoter, T-bet promoter, IFNgamma promoter, TIM3 promoter, IL4 promoter, GATA3 promoter, IL1 promoter, IL5 promoter, IL6 promoter, IL13 promoter, IL10 promoter, IL17A promoter, IL6 promoter, IL21 promoter, IL23R promoter, FoxP3 promoter, CTLA4 promoter, CD25 promoter, PD1 promoter, CD45RO promoter, CCR7 promoter, CD28 promoter, CD95 promoter, CD28 promoter, CD27 promoter, CD127 promoter, PD-1 promoter, CD122 promoter, CD132 promoter, c-Kit promoter, nuclear factor of activated T cells (NFAT) promoter, programmed death 1 (PD-1) promoter, T cell immunoglobulin mucin-3 (TIM-3) promoter, cytotoxic T lymphocyte antigen-4 (CTLA4) promoter, lymphocyte-activation protein 3 (LAG-3) promoter, tumor necrosis factor (TNF)-related apoptosis-inducing ligand (TRAIL) promoter, B- and T-lymphocyte attenuator (BTLA) promoter, CD25 promoter, CD69 promoter, Fas ligand (FasL) promoter, TIGIT promoter, TGF-beta promoter, T-bet promoter, Eomes promoter, GATA3 promoter, CD45RA promoter, 2B4 promoter, Type I interferon (IFN) alpha, Type I IFN beta promoter, IFN gamma promoter, IRF3 promoter, IRF7 promoter, NFkB promoter, AP-1 promoter, TNF-alpha promoter, CD130 promoter, NR4A1 promoter, NR4A2, or NR4A3 promoter.

In one aspect, the disclosure provides a vector comprising a recombinant nucleic acid construct as described herein. In some embodiments, the vector is a viral vector selected from a retrovirus vector, an adenovirus vector, and an adeno-associated virus vector. In some embodiments, the retrovirus is a lentivirus.

In one aspect, the disclosure provides a cell comprising a polypeptide described herein and/or a recombinant nucleic acid as described herein. In some embodiments, the ell is a non-natural cell or has been genetically engineered. In some embodiments, the cell is not a CD4+ T cell. In some embodiments, the cell is not a cancerous cell. In some embodiments, the recombinant nucleic acid is exogenous.

In some embodiments, the cell comprises at least one copy, or at least two copies, of endogenous nucleic acid sequence encoding a CARD11 protein, or a protein comprising a CARD11 CARD domain without any SH2 domain. In some embodiments, the recombinant nucleic acid of the cell is located at the endogenous CARD11-encoding gene locus, or comprises at least a portion of the endogenous CARD11-encoding gene, of the cell. I

In some embodiments, the cell comprises a polypeptide comprising a sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204, SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ ID NO: 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, and functional variants thereof comprising at least one mutation listed in Table 1.

In some embodiments, the cell comprises a nucleic acid sequence having at least 75%, 80%, 85%, 90%, 95%, 96%, 97%, 98%, 99% or 100% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, or functional variants thereof comprising at least one mutation listed in Table 1.

In some embodiments, the cell further comprises: (i) a chimeric antigen receptor (CAR) having specificity for a target antigen; and/or (ii) a T cell receptor (TCR) having specificity for a target antigen. In some embodiments, the cell is selected from the group consisting of an immune cell, a T cell, a regulatory T cell, a CD8+ cell, a natural killer cell, a tumor infiltrating lymphocyte, and a MAIT cell. In some embodiments, the target antigen is DLL3, LY6G6D, Claudin 6, GCC, p53R175H, or PRAME.

In one aspect, the disclosure provides a method of preparing a T cell for use in a cell therapy, the method comprises expressing in the T cell a polypeptide as described herein. In some embodiments, the method comprises genetically modifying the T cell for expression of the polypeptide. In some embodiments, the method comprises introducing to the T cell a recombinant nucleic acid encoding the polypeptide, or a vector comprising the recombinant nucleic acid. In some embodiments, the method comprises expressing in the T cell an engineered immune receptor that binds to a target in a tumor cell. In some embodiments, the method comprises administering to the subject a cell as described herein, or a T cell prepared by a method described herein.

In some embodiments, the subject has a cancer or an autoimmune disease. In some embodiments, the cancer is a solid tumor. In some embodiments, the cancer is a hematological cancer. In some embodiments, the cancer expresses CD19, B7H3 (CD276), BCMA (CD269), ALPPL2, Claudin 18.2, CD123, CD171, CD179a, CD20, CD213A2, CD22, CD24, CD246, CD272, CD30, CD33, CD38, CD44v6, CD46, CD71, CD97, CEA, Claudin 6(CLDN6), CLECL1, CS-1, DLL-3, EGFR, EGFRvIII, ELF2M, EpCAM, EphA2, Ephrin B2, FAP, FLT3, GCC, GD2, GD3, GM3, GPRC5D, HER2 (ERBB2/neu), IGLL1, IL-11Ra, KIT (CD117), KLK2, LY6G6D, MUC1, NCAM, p53R175H, PAP, PDGFR-β, PRAME, PRSS21, PSCA, PSMA, ROR1, SIRPα, SSEA-4, TAG72, TEM1/CD248, TEM7R, TSHR, VEGFR2, ALPI, citrullinated vimentin, cMet, and/or Axl. In some embodiments, the cancer expresses DLL3, LY6G6D, Claudin 6, GCC, p53R175H, and/or PRAME.

In some embodiments, the cancer is small cell lung cancer, colorectal cancer, testicular cancer, ovarian cancer, melanoma, lymphoma, leukemia, multiple myeloma, prostate cancer, breast cancer, non-small cell lung cancer, gastric cancer, esophageal cancer, liver cancer, kidney cancer, head & neck cancer, glioblastoma, neuroblastoma, soft tissue sarcoma, uterine cancer, brain cancer, skin cancer, renal cancer, bladder cancer, pancreatic cancer, thyroid cancer, eye cancer, gastrointestinal cancer, carcinoma, or sarcoma.

In some embodiments, the method of treatment does not comprise administration of lymphodepletive agents within 7 days prior to administration of the T cell therapy. In some embodiments, the method of treatment does not comprise administration of cyclophosphamide, fludarabine, and/or bendamustine within 7 days prior to administration of the T cell therapy. In some embodiments, the method of treatment does not comprise administration of at least 600,000 IU/kg of IL-2 every 8 hours. In some embodiments, the method of treatment does not comprise a checkpoint therapy which blocks PD-1 or CTLA-4 signaling.

In some embodiments, the cell has reduced exhaustion, increased proliferative capacity, enhanced replicative lifespan, decreased replicative senescence, enhanced anti-tumor effect, reduced dysfunction, enhanced persistence, and/or increase intratumoral presence in vivo. In some embodiments, the cell has increased or decreased signaling through the CARD11-BCL10-MALT1 complex, NF-κB, AP-1, NFAT, JAK/STAT, and/or MEK/ERK pathways.

In some embodiments, the disclosure provides a recombinant nucleic acid construct encoding a polypeptide, wherein the polypeptide includes a mutation capable of altering (i) T cell signaling through NFAT, NF-κB and/or AP-1 pathways, (ii) cytokine production, and/or (iii) in vivo persistence in tumors of therapeutic T cells comprising the mutation.

In some embodiments of the recombinant nucleic acid constructs and/or recombinant nucleic acids of the disclosure, the mutation is a point mutation, a gene fusion, a substitution, a gain-of-function mutation, a stop-gain mutation, an insertion mutation, a deletion mutation, a duplication mutation or a translocation. In some embodiments, the mutation is a T cell lymphoma mutation or a mutation in a clonally expanded population of T cells. In some embodiments, the mutation is capable of altering/promoting/enhancing CARD11-BCL10-MALT1 complex signaling in T cells.

In some embodiments, the mutation is in a gene selected from the group consisting of: caspase recruitment domain family member 11 (CARD11), capping protein regulator and myosin 1 linker 2 (CARMIL2), mucosa-associated lymphoid tissue lymphoma translocation protein 1 (MALT1), B-cell lymphoma 6 (BCL6), B-cell lymphoma 10 (BCL10) and MYCN.

In some embodiments, the polypeptide includes an amino acid sequence having at least about 75%, 80%, 85%, 90%, 95%, 96%, 97%, 98%, 99% or 100% identity to SEQ ID NO: 25 and comprising a substitution at an amino acid position selected from the group consisting of 361, 615, 634, 655, and 357 of SEQ ID NO: 25. In some embodiments, the substitution includes Y361C, S615F, E634K, D357N, S655C, or combinations thereof.

In some embodiments, the mutation is a fusion between a CARD11 polypeptide and a PIK3R3 polypeptide. In some embodiments, the fusion includes a CARD domain, a coiled-coil domain, and an SH2 domain from PIK3R3.

In some embodiments of the recombinant nucleic acid constructs and/or recombinant nucleic acids nucleic acid constructs and/or recombinant nucleic acids of the disclosure, the polypeptide is encoded by a nucleic acid sequence having at least about 75%, 80%, 85%, 90%, 95%, 96%, 97%, 98%, 99%, or 100% sequence identity to SEQ ID NO: 205, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 246, SEQ ID NO: 247, SEQ ID NO: 249, SEQ ID NO: 251, SEQ ID NO: 253, or SEQ ID NO: 255.

In some embodiments of the recombinant nucleic acid constructs and/or recombinant nucleic acids of the disclosure, the polypeptide includes an amino acid sequence having at least about 75%, 80%, 85%, 90%, 95%, 96%, 97%, 98%, 99% or 100% sequence identity to SEQ ID NO: 206, SEQ ID NOS: 226, SEQ ID NOS: 228, SEQ ID NOS: 230, SEQ ID NOS: 232, SEQ ID NOS: 234, SEQ ID NOS: 236, SEQ ID NOS: 238, SEQ ID NOS: 240, SEQ ID NOS: 242, SEQ ID NOS: 244, SEQ ID NOS: 248, SEQ ID NOS: 250, SEQ ID NOS: 252, SEQ ID NOS: 254, or SEQ ID NO:256.

In some embodiments of the recombinant nucleic acid constructs and/or recombinant nucleic acids of the disclosure, the polypeptide includes an amino acid sequence having at least about 75%, 80%, 85%, 90%, 95%, 96%, 97%, 98%, 99% or 100% sequence identity to SEQ ID NO: 4 and comprising a substitution at amino acid 647 of SEQ ID NO: 4, SEQ ID NO: 28 and having a substitution at amino acid 575 of SEQ ID NO: 28, or SEQ ID NO: 114 and having a substitution at amino acid 44 of SEQ ID NO: 114. In some embodiments, the substitution at amino acid 647 of SEQ ID NO: 4 is S647R, wherein the substitution at amino acid 575 of of SEQ ID NO: 28 is Q575E, and wherein the substitution at amino acid 44 of SEQ ID NO: 114 is P44L.

In some embodiments of the recombinant nucleic acid constructs and/or recombinant nucleic acids of the disclosure, the polypeptide includes a mutation capable of (i) altering JAK/STAT signaling in T cells (ii) altering cytokine production, and/or (iii) enhancing in vivo persistence of therapeutic T cells comprising the mutation in tumors. In some embodiments, the polypeptide having the mutation includes a JAK1, JAK3, STAT3, or STAT5 polypeptide. In some embodiments, the polypeptide includes an amino acid sequence having at least about 75%, 80%, 85%, 90%, 95%, 96%, 97%, 98%, 99% or 100% sequence identity to: SEQ ID NO: 90 and having a substitution at amino acid position 1097 of SEQ ID NO: 90, or to SEQ ID NO: 94 and having a substitution at an amino acid position 573 of SEQ ID NO: 94, or having SEQ ID NO: 176 and having a substitution at an amino acid position 618, 647, or 661 of SEQ ID NO: 176, or SEQ ID NO: 141 and having a substitution at an amino acid position 628, or at amino acid position 665 of SEQ ID NO: 182.

In some embodiments of the recombinant nucleic acid constructs and/or recombinant nucleic acids of the disclosure, the nucleic acid construct includes a nucleic acid sequence having at least about 75%, 80%, 85%, 90%, 95%, 96%, 97%, 98%, 99% or 100% sequence identity to a sequence selected from the group consisting of SEQ ID NO:87, SEQ ID NO: 91, SEQ ID NO:169, SEQ ID NO: 171, SEQ ID NO: 173, SEQ ID NO: 177 or SEQ ID NO: 179.

In some embodiments of the recombinant nucleic acid constructs and/or recombinant nucleic acids of the disclosure, the polypeptide comprises a mutation capable of altering co-stimulatory molecule signaling in T cells and persistence in tumors of T cells comprising the mutation. In some embodiments, the polypeptide having the mutation includes a TNFRSF1B, CD28, ICOS, or CTLA4 polypeptide. In some embodiments, the polypeptide includes an amino acid sequence having at least about 75%, 80%, 85%, 90%, 95%, 96%, 97%, 98%, 99% or 100% sequence identity to i) SEQ ID NO: 192 and including a substitution at an amino acid position 256 or at position 377 of SEQ ID NO: 192, or ii) SEQ ID NO: 42 and including a substitution at an amino acid position 51 or 77 of SEQ ID NO: 42, or iii) SEQ ID NO: 220, or iv) SEQ ID NO: 218. In some embodiments, the nucleic acid construct includes a polypeptide having an amino acid sequence having at least about 75%, 80%, 85%, 90%, 95%, 96%, 97%, 98%, 99% or 100% sequence identity to SEQ ID NO: 189, SEQ ID NO: 189, SEQ ID NO: 35, SEQ ID NO: 37, SEQ ID NO: 39, SEQ ID NO:43, SEQ ID NO:217 or SEQ ID NO:219.

In some embodiments, the polypeptide includes a mutation capable of altering RAS/MEK/ERK signaling in T cells and in vivo persistence in tumors of therapeutic T cells comprising the mutation.

In some embodiments, the mutation includes a BRAF gene or a RASGRP1 polypeptide. In some embodiments, the polypeptide includes an amino acid sequence having at least about 75%, 80%, 85%, 90%, 95%, 96%, 97%, 98%, 99% or 100% sequence identity to i) SEQ ID NO: 16 and including an amino acid substitution at amino acid position 469 or at position 594 of of SEQ ID NO: 16, or to SEQ ID NO: 158 and including a substitution at amino acid position 261 of SEQ ID NO: 261. In some embodiments, the nucleic acid construct comprises a nucleic acid sequence having at least about 75%, 80%, 85%, 90%, 95%, 96%, 97%, 98%, 99% or 100% sequence identity to SEQ ID NO: 9, SEQ ID NO: 11, SEQ ID NO:13 or SEQ ID NO: 157.

In some embodiments, the polypeptide includes a mutation capable of altering phospholipase gamma signaling and/or (ii) cytokine production, and/or (iii) in vivo persistence in tumors of therapeutic T cells comprising the mutation. In some embodiments, the mutation is in a Phospholipase C gamma 1 (PLCG1) gene. In some embodiments, the polypeptide includes an amino acid sequence having at least about 75%, 80%, 85%, 90%, 95%, 96%, 97%, 98%, 99% or 100% sequence identity to SEQ ID NO: 142 and having a substitution at amino acid position 47, 48, 520, 1163 or 1165 of SEQ ID NO: 142. In some embodiments, the recombinant nucleic acid construct includes a nucleic acid sequence having at least about 75%, 80%, 85%, 90%, 95%, 96%, 97%, 98%, 99% or 100% sequence identity to SEQ ID NO: 133, SEQ ID NO: 135, SEQ ID NO: 137, SEQ ID NO:139, or SEQ ID NO: 143.

In some embodiments of the recombinant nucleic acid constructs and/or recombinant nucleic acids of the disclosure, the polypeptide includes a mutation capable of altering a transcription factor activity in T cells comprising the mutation. In some embodiments, the polypeptide having the mutation includes a NFKB1, NFKB2 or JUNB polypeptide. In some embodiments, the polypeptide includes an amino acid sequence having at least about 75%, 80%, 85%, 90%, 95%, 96%, 97%, 98%, 99% or 100% sequence identity to SEQ ID NO: 118 and comprising a substitution at amino acid 67 of SEQ ID NO: 118, SEQ ID NO: 122 and comprising a substitution at amino acid 565 of SEQ ID NO: 122, or SEQ ID NO: 98 and comprising a substitution at amino acid 282. In some embodiments, the nucleic acid construct comprises a nucleic acid sequence having at least about 75%, 80%, 85%, 90%, 95%, 96%, 97%, 98%, 99% or 100% sequence identity to SEQ ID NO:115, SEQ ID NO: 119 and SEQ ID NO: 95.

In some embodiments of the constructs polypeptides and nucleic acids of the disclosure, the mutation is a mutation listed in Table 1.

In some embodiments of the recombinant nucleic acid constructs and/or recombinant nucleic acids of the disclosure, the polypeptide includes a sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204, SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ ID NO: 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, or functional variants thereof having at least one mutation listed in Table 1.

In some embodiments of the recombinant nucleic acid constructs and/or recombinant nucleic acids of the disclosure, the recombinant nucleic acid construct includes a nucleic acid sequence having at least about 75%, 80%, 85%, 90%, 95%, 96%, 97%, 98%, 99% or 100% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, or functional variants thereof comprising at least one mutation listed in Table 1.

In an aspect of the disclosure, provided herein is a recombinant nucleic acid construct encoding a fusion polypeptide wherein the fusion polypeptide includes a first polypeptide encoding a partial CARD11 polypeptide and a second polypeptide encoding a partial PIK3R3 polypeptide and wherein expression of the fusion polypeptide promotes in vivo persistence in tumors of therapeutic T cells comprising the fusion polypeptide.

In some embodiments, the T cell is selected from the group consisting of a regulatory (Treg), a gamma delta T cell, an invariant iNKT cell, a macrophage, a monocyte, a natural killer (NK), a CAR T cell, and an engineered T cell comprising a transcriptional receptor.

In some embodiments of the recombinant nucleic acid constructs and/or recombinant nucleic acids of the disclosure, the first polypeptide includes an amino acid sequence having at least about 75%, 80%, 85%, 90%, 95%, 96%, 97%, 98%, 99% or 100% sequence identity to SEQ ID NO: 258. In some embodiments, the second polypeptide comprises an amino acid sequence having at least about 75%, 80%, 85%, 90%, 95%, 96%, 97%, 98%, 99% or 100% sequence identity to SEQ ID NO: 260. In some embodiments, the first polypeptide includes a CARD domain, a coiled-coil domain, and wherein the second polypeptide comprises an SH2 domain from PIK3R3.

In some embodiments of the recombinant nucleic acid constructs and/or recombinant nucleic acids of the disclosure, the construct comprises a promoter. In some embodiments, the promoter is a constitutive promoter. In some embodiments, the constitutive promoter is a CD4 promoter, CD8a promoter, CD8b promoter, TCRa promoter, TCRb promoter, CD3d promoter, CD3g promoter, CD3e promoter, or CD3z promoter.

In some embodiments, the promoter is a MND promoter, EF1a promoter, sEF1a promoter, gamma retroviral LTR promoter, minimal TATA promoter, a pGK, actin promoter, CD25 promoter, IL2 promoter, IL7 promoter, IL15 promoter, KLRG-1 promoter, HLA-DR promoter, CD38 promoter, CD69 promoter, Ki-67 promoter, CD11a promoter, CD58 promoter, CD99 promoter, CD62L promoter, CD103 promoter, CCR4 promoter, CCR5 promoter, CCR6 promoter, CCR9 promoter, CCR10 promoter, CXCR3 promoter, CXCR4 promoter, CLA promoter, Granzyme A promoter, Granzyme B promoter, Perforin promoter, CD57 promoter, CD161 promoter, IL-18Ra promoter, CD69 promoter, GzmB promoter, T-bet promoter, IFNgamma promoter, TIM3 promoter, IL4 promoter, GATA3 promoter, IL1 promoter, IL5 promoter, IL6 promoter, IL13 promoter, IL10 promoter, IL17A promoter, IL6 promoter, IL21 promoter, IL23R promoter, FoxP3 promoter, CTLA4 promoter, CD25 promoter, PD1 promoter, CD45RO promoter, CCR7 promoter, CD28 promoter, CD95 promoter, CD28 promoter, CD27 promoter, CD127 promoter, PD-1 promoter, CD122 promoter, CD132 promoter, c-Kit promoter, nuclear factor of activated T cells (NFAT) promoter, programmed death 1 (PD-1) promoter, T cell immunoglobulin mucin-3 (TIM-3) promoter, cytotoxic T lymphocyte antigen-4 (CTLA4) promoter, lymphocyte-activation protein 3 (LAG-3) promoter, tumor necrosis factor (TNF)-related apoptosis-inducing ligand (TRAIL) promoter, B- and T-lymphocyte attenuator (BTLA) promoter, CD25 promoter, CD69 promoter, Fas ligand (FasL) promoter, TIGIT promoter, TGF-beta promoter, T-bet promoter, Eomes promoter, GATA3 promoter, CD45RA promoter, 2B4 promoter, Type I interferon (IFN) alpha, Type I IFN beta promoter, IFN gamma promoter, IRF3 promoter, IRF7 promoter, NFkB promoter, AP-1 promoter, TNF-alpha promoter, CD130 promoter, NR4A1 promoter, NR4A2, or NR4A3 promoter.

In an aspect of the disclosure, provided herein are recombinant nucleic acid constructs and/or recombinant nucleic acids wherein the cytokine is IL-2, IL-4, 11-5, TNF alpha, IFN-gamma, IL-13 and/or a combination of any thereof.

In an aspect of the disclosure, provided herein are vectors including any of the recombinant nucleic acid constructs and/or recombinant nucleic acids of the disclosure. In some embodiments, the vector is a retrovirus, an adenovirus, or an adeno-associated virus. In some embodiments, the retrovirus is a lentivirus.

In an aspect of the disclosure, provided herein are cells including a nucleic acid construct or a vector of the disclosure. In some embodiments, the cell includes (i) a chimeric antigen receptor (CAR) having specificity for a target antigen; (ii) a T cell receptor (TCR) having specificity for a target antigen; and/or (iii) a transcriptional receptor.

In some embodiments, the cells are selected from the group consisting of an immune cell, a T cell, a regulatory T cell, a CD8+ cell, a natural killer cell, and a tumor infiltrating lymphocyte.

In an aspect, the disclosure provides a target antigen. In some embodiments, the target antigen is selected from the group consisting of DLL3, LY6G6D, Claudin 6, GCC, p53R175H, PRAME, CD1, CD1a, CD1b, CD1c, CD1d, CD1e, CD2, CD3d, CD3e, CD3g, CD3F, CD4, CD5, CD7, CD8a, CD8b, CD19, CD20, CD21, CD22, CD23, CD24, CD25, CD27, CD28, CD30, CD33, CD34, CD38, CD40, CD44v6, CD45, CD48, CD52, CD59, CD66, CD70, CD71, CD72, CD73, CD79A, CD79B, CD80 (B7.1), CD86 (B7.2), CD94, CD95, CD97, CD123, CD134, CD140 (PDGFR4), CD152, CD154, CD158, CD171, CD178, CD179, CD179a, CD181 (CXCR1), CD182 (CXCR2), CD183 (CXCR3), CD210, CD246, CD252, CD253, CD261, CD262, CD273 (PD-L2), CD274 (PD-L1), CD276 (B7H3), CD279, CD295, CD339 (JAG1), CD340 (HER2), CEA, CLL-1, CS1, EGFR, FGFR2, AFP, CA125, MUC-1, MAGE, alkaline phosphatase, placental-like 2 (ALPPL2), B-cell maturation antigen (BCMA), green fluorescent protein (GFP), enhanced green fluorescent protein (eGFP), Claudin18.2, PSMA, ROR1, Mesothelin, IL13Ra2, FAP, signal regulatory protein α (SIRPα), TRAC, TCRβ, BCMA, TSHR, EGFRvIII, GD2, GD3, Tn Ag, ROR1, ROR2, GPC1, GPC2, FLT3, FAP, TAG72, CEA, EPCAM, B7H3, KIT, IL-13Ra2, IL-11Ra, PSCA, PRSS21, VEGFR2, LewisY, PDGFR-beta, SSEA-4, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-la, MAGE-Al, legumain, HPV E6, E7, MAGE Al, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, LAIR1, FCAR, LILRA2, CD300LF, CLEC12A, BST2, EMR2, LY75, GPC3, FCRL5, IGLL1, AFP, TRAC, TCRβ, BCMA, TSHR, EGFRvIII, GD2, GD3, Tn Ag, PSMA, ROR1, ROR2, GPC1, GPC2, FLT3, FAP, TAG72, CEA, EPCAM, B7H3, KIT, IL-13Ra2, IL-11Ra, PSCA, PRSS21, VEGFR2, LewisY, PDGFR-beta, SSEA-4, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-la, MAGE-Al, legumain, HPV E6,E7, MAGE Al, ETV6-AML, sperm protein 17, XAGE1, Tie 2, MAD-CT-1, MAD-CT-2, Fos-related antigen 1, p53, p53 mutant, KRAS, mutant KRAS, KRAS G12D, prostein, surviving, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, LAIR1, FCAR, LILRA2, CD300LF, CLEC12A, BST2, EMR2, LY75, GPC3, FCRL5, IGLL1, the extracellular portion of the APRIL protein, or any combinations thereof.

In some embodiments, the target antigen is selected from cell surface receptors, adhesion proteins, integrins, mucins, lectins, tumor-associated antigens, and tumor-specific antigens. In some embodiments, the target antigen is a tumor-associated antigen selected from the group consisting of CD19, B7H3 (CD276), BCMA (CD269), ALPPL2, Claudin 18.2, CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, IL-11Ra, KIT (CD117), MUC1, NCAM, PAP, PDGFR-0, PRSS21, PSCA, PSMA, ROR1, SIRPα, SSEA-4, TAG72, TEM1/CD248, TEM7R, TSHR, VEGFR2, ALPI, citrullinated vimentin, cMet, and Axl.

In a further aspect, the disclosure provides methods of preparing a T cell for use in a cell therapy. In some embodiments, the method comprises transducing the T cell with a recombinant nucleic acid construct comprising a mutation capable of altering (i) T cell signaling through NFAT, NF-κB and/or AP-1 pathways, (ii) cytokine production, and/or (iii) in vivo persistence of T cells in tumors. In some embodiments, the recombinant nucleic acid construct includes the recombinant nucleic acid construct of the disclosure.

In some embodiments, the T cell further includes a CAR, a TCR, or a transcriptional receptor.

In a further aspect, the disclosure provides a method for enhancing the in vivo persistence of a T cell in a subject in need thereof, the method including administering to the subject a therapeutically effective amount of the T cell of the disclosure. In some embodiments, the T cell is selected from the group consisting of a regulatory (Treg), a natural killer (NK), a tumor infiltrating lymphocyte, and a CAR T cell.

In some embodiments, the subject has cancer or an autoimmune disease. In some embodiments, the cancer is a solid tumor. In some embodiments, the cancer is a hematological cancer.

Also provided herein, is a method for identifying a mutation useful for improving T cell therapy including a) identifying mutations from a clonal T cell genomic sequencing database; b) identifying a frequency of occurrence of the mutations; and c) applying a statistical test to identify a significant difference in a hotspot genomic region where a mutation occurs more often than expected by chance, thereby identifying a mutation in the hotspot region that is capable of improving T cell therapy. In some embodiments, the statistical test includes using a binomial distribution, a Chi square analysis or any other multivariate analysis. In some embodiments, the mutation is a T cell lymphoma mutation. In some embodiments, the hotspot region is in a coding sequence of a gene.

In some embodiments, the mutation improves T cell therapy by a) increasing proliferation, and/or b) altering effector function, and/or c) resisting T cell dysfunction, and/or d) enhancing growth of a therapeutic T cell comprising the mutation in a tumor. In some embodiments, the mutation is a mutation listed in Table 1. In some embodiments, the mutation promotes positive T cell selection and/or T cell clonal outgrowth.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIG. 1 is a schematic depicting the present disclosure's approach to creation of a screening library of mutations identified in T cell lymphomas, and in vitro and in vivo screening analysis of the library.

FIGS. 2A-2G show the detailed signaling results of the in vitro T cell lymphoma mutation screen. FIG. 2A-2D show the reporter activity Z score for NFAT, NF-κb and AP-1 for each of the indicated T cell lymphoma mutation constructs. In some cases the PD-1 expression and IL-2 Z scores are also depicted. PD-1 expression was assessed via flow. IL-2 secretion was assessed via ELISA. Z score indicates the mean Z score of two independent biological replicates. FIG. 2A shows the reporter activity for each construct when expressed in CD19-CD28z CAR Jurkat cells and co-cultured with K562 (CD19 negative) cell lines. FIG. 2B shows the reporter activity and IL-2 secretion for each construct when expressed in CD19-CD28z CAR Jurkat cells and co-cultured with CD19-K562 positive cell lines. FIG. 2C shows the reporter activity and PD-1 expression for each construct when expressed in CD19-BBz CAR Jurkat cells and co-cultured with K562 (CD19 negative) cell lines. FIG. 2D shows the reporter activity, IL-2 secretion and PD-1 expression for each construct when expressed in CD19-BBz CAR Jurkat cells and co-cultured with CD19-K562 positive cell lines. FIG. 2E shows a bar graph demonstrating that in both the CD19-CD28z CAR and CD19-BBz CAR settings, point mutation and fusion constructs showed more significantly different effects versus controls in the CD19-K562 condition compared to the parental K562 (CD19 negative) line. FIG. 2F shows a bar graph indicating the number of T cell lymphoma point mutation or fusion constructs for which expression in CD19-BBz CAR Jurkats resulted in each distinct combination of signaling up or downregulation upon antigenic stimulation. FIG. 2G shows a bar graph indicating percent NFAT, NF-κB, and AP-1 signaling in CD19-BBz CAR cells upon CD19-K562 stimulation of select mutations highlighting a high degree of tunability of signaling outputs. Each point represents a biological replicate.

FIG. 3 shows the Z score for NFAT, AP-1 and NF-κB signaling of mutations that showed a statistically significant difference from their wildtype controls, ordering the mutations based on the Z-score from positive (top of y-axis) to negative (bottom of y-axis). This ordering demonstrates that some mutations induce a gain of function (increase the signaling output as compared to wildtype), while others cause a loss of function (reduce the signaling output as compared to wildtype).

FIG. 4 shows tumor growth curves of CD19-K562 tumor bearing animals treated with PBS or CD19-BBz CAR T cells.

FIGS. 5A and 5B show the correlation between results of the in vivo screen and PD-1 in vitro studies on the identified mutations of the disclosure. FIG. 5A shows a waterfall plot of the in vivo screen log 2 fold change for each construct as determined by MAGeCK, compared to baseline (pre-injection). Positive log 2 fold change indicates increased persistence in tumors compared to input. FIG. 5B depicts a bar graph plotting the in vivo fold change of each mutation and splitting constructs into two categories 1) all constructs that did not significantly increase PD-1 expression in the in vitro jurkat screen or 2) constructs that did significantly increase PD-1 expression in the in vitro jurkat screen. These results indicate that PD-1 expression in vitro may help to predict in vivo persistence of T cell lymphoma mutations.

FIG. 6 is a schematic indicating signaling by the CARD11-BCL10-MALT1 (CBM) complex in T cells. In normal T cells, T cell receptor (TCR) signaling activates PKCθ, which in turn promotes the assembly of the CBM signalosome. The CBM complex subsequently has three major outputs: NF-κB transcriptional activity, AP-1 transcriptional activity and MALT1 proteolytic activity.

FIG. 7 shows a schematic of the CARD11-PIK3R3 fusion according to the disclosure. The topmost diagram shows the structure and domains of wild-type CARD11 comprising 1154 amino acids and the location of the breakpoint where the C-terminal component is lost and the PIK3R3 polypeptide is attached in the fusion protein. The middle diagram depicts the structure of wild-type PIK3R3 which comprises 461 amino acids and the location of the breakpoint where the C-terminal component of PIK3R3 is attached in the fusion protein. The bottom diagram depicts the CARD11-PIK3R3 fusion protein having 725 amino acids with the CARD, Coiled-coil and truncated inhibitory domains derived from the N-terminus of CARD11 and an SH2 domain derived from the C-terminus of PIK3R3. The truncation of CARD11 is at the location in the wildtype gene shown in topmost structure and labelled “Breakpoint”, and the truncation of PIK3R3 is at the location in the wildtype gene shown in the middle structure and labelled “Breakpoint”.

FIGS. 8A-B depict various CARD11-PIK3R3 fusion variants and their function. FIG. 8A is a diagram of the variants harboring deletions of various domains of the CARD11-PIK3R3 fusion generated. FIG. 8B depicts reporter activity of CD19-BBz CAR CARD11-PIK3R3 fusion variants in Jurkat cells co-cultured with CD19-K562s.

FIGS. 9A-C illustrate CARD11-PIK3R3 fusion function in the CBM complex. FIG. 9A is a diagram of the BCL10 binding-deficient CARD11-PIK3R3 mutant containing an alanine substitution for arginine at the 28th amino acid position. FIG. 9B shows the CD19-BBz CAR Jurkat reporter activity of the BCL10 binding-deficient CARD11-PIK3R3 mutant compared to control and CARD11-PIK3R3 expressing cells. **** indicates P value<0.0001, determined by one-way ANOVA followed by Tukey's multiple comparison test. FIG. 9C shows western blotting of MALT1 substrates in CARD11-PIK3R3 expressing CD19-BBz CAR Jurkat cells or control cells. P/I indicates phorbol myristate acetate/ionomycin treatment. Ct indicates the C-terminal cleavage product. Data representative of 2 independent experiments.

FIGS. 10A-B show CARD11-PIK3R3 fusion phosphorylation signaling dynamics. FIG. 10A shows a schematic indicating an experimental approach to study CAR-dependent signaling in primary human CD3+ T cells in accordance with the current disclosure. FIG. 10B shows heatmaps of marker expression determined by mass cytometry by time of flight (CyTOF) for each of the indicated timepoints in CD19-BBz CAR samples. Values indicate mean for three independent T cell donors.

FIGS. 11A-C show CARD11-PIK3R3 fusion transcriptional landscape. FIG. 11A shows the principal component (PC) analysis of human CD8+ T cells from three independent donors transduced with the indicated constructs and left unstimulated (Unstim) or co-cultured for 8 hours with CD19 expressing A549 cells (Stim). FIG. 11B shows a volcano plot of differentially expressed genes in CD19-BBz CAR T cells either unstimulated or stimulated with CD19 antigen. Select genes of interest are labeled. Positive log 2 fold changes indicate higher expression in CARD11-PIK3R3 expressing CAR-T cells. FIG. 11C shows NF-κB, AP-1, and MALT1 gene signature enrichment in CARD11-PIK3R3 expressing CD19-BBz CAR cells after stimulation with CD19 expressing A549 cells.

FIGS. 12A-B show the results of experiments of CD3+ T cell growth after anti-CD3/anti-CD28 bead stimulation. Cells counts of CD19-BBz CAR (FIG. 12A) or CD19-CD28z CAR (FIG. 12B) CD3+ T cells over a period of 9 days after removal from anti-CD3/anti-CD28 bead stimulation. Ns indicates not significant, * indicates P value<0.05.

FIGS. 13A-B show CD3+ cytokine secretion after co-culture with CD19-K562s. Cytokine secretion profiles of CD19-BBz CAR (FIG. 13A) or CD19-CD28z CAR (FIG. 13B) CD3+ T cells co-cultured for 48 hours at 1:1 ratio with CD19-K562s. P values determined by ratio paired T test. Ns indicates not significant, * indicates P value<0.05, ** indicates P value<0.01.

FIGS. 14A-C shows activation and cytokine secretion of CD8+ T cells after co-culture with CD19-K562s. FIG. 14A shows activation markers expressed on CD19-BBz CAR T cells, with or without CARD11-PIK3R3, 24 hours post 1:1 co-culture with CD19-K562s. FIG. 14B shows activation markers expressed on untransduced or CARD11-PIK3R3 transduced CD8+ T cells 24 hours post 1:1 co-culture with CD19-K562s. FIG. 14C shows cytokine secretion of CD8+ T cells expressing the CD19-BBz CAR, with or without CARD11-PIK3R3, 48 hours post 1:1 co-culture with CD19-K562s. P values determined by ratio paired T test. Ns indicates not significant, * indicates P value<0.05.

FIGS. 15A-B show the results of experiments on long co-culture persistence, killing and multi-stimulation growth. FIG. 15A shows flow plots depicting CD19-BBz CAR transduced CD8+ T cells and CD19-K562 populations after 14-day co-culture together with and without supplemental IL-2, one donor depicted. Bar graph summarizing population percentages from three donors. P values determined by unpaired T test. FIG. 15B shows cell counts of CD19-BBz CAR transduced CD8+ T cells co-cultured with CD19-K562s on Day 0 and restimulated with targets on Day 6. P values determined by unpaired T test. Ns indicates not significant. * indicates P value<0.05, **** indicates P value<0.0001.

FIGS. 16A and B show cytotoxicity at varying effector to target ratios. Growth of CD19-A549 mKate2+ targets co-cultured with CD19-BBz CAR (A) or CD19-CD28z CAR (B) CD8+ T cells over 108 hour period. Bar graph indicates target cell counts at hour 108, standardized to control. P values calculated by 1-way ANOVA followed by Tukey's multiple comparison test. ** indicates P value<0.01, **** indicates P value<0.0001.

FIG. 17 shows a schematic indicating a xenograft CAR leukemic model used to assess CARD11-PIK3R3 therapeutic function in T cells in vivo. In this model NOD.Cg-Prkdcscid Il2rgtm1Wjl/SzJ (NSG) mice are dosed with 5×10′ Nalm6-GFP-Luciferase cells via tail vein injection. Primary human CD3+ T cells are transduced to express the CD19-BBz CAR (with or without co-expression of CARD11-PIK3R3) and electroporated with TRAC RNPs to knockout expression of the human TCR. TRAC RNPs are CRISPR/Cas9 ribonuclear protein complexes targeting the human T cell receptor alpha constant region gene. Nalm6 bearing mice are dosed with 1×10⁶ CD19-BBz CAR T cells and tumor burden is assessed twice weekly via bioluminesce imaging using an IVIS imaging system. Mice are euthanized at first signs of hind limb paralysis.

FIG. 18A-B demonstrate that CD19-BBz CAR expressing the CARD11-PIK3R3 fusion control Nalm6 leukemia in vivo. FIG. 18A shows average radiance of luciferase expressing Nalm6 tumor cells measured using in vivo imaging, radiance used as a proxy for tumor burden. Data indicates mean+/−standard deviation. P values determined using unpaired T test of average radiance values at Day 21. Control (n=5), CAR (n=6), CAR+CARD11-PIK3R3 (n=5). ** indicates P value<0.01. FIG. 18B shows survival analysis of Nalm6 bearing mice treated with untransduced (control)(n=5), BBz CAR (n=6) or BBz CAR with CARD11-PIK3R3 (n=5) T cells. P values determined using Log-rank (Mantel-Cox) test. *** indicates P values<0.001.

FIG. 19 is a schematic of the syngeneic CAR Melanoma Model used to assess CARD11-PIK3R3 therapeutic function in CD19-BBz CAR T cells in vivo. hCD19-B16s=B16 tumor cells expressing human CD19. OT-I cells=T cells collected from C57BL/6-Tg(TcraTcrb)1100Mjb/J mice

FIGS. 20A-C shows results of experiments with CD19-BBz CAR with CARD11-PIK3R3 fusion in controlling hCD19 B16 tumors. FIG. 20A shows accumulation of CD19-BBz CAR T cells in the spleen and tumor 5 days post adoptive cell transfer. P values determined using unpaired Mann-Whitney t test. FIGS. 20B and C show tumor volumes (B) and survival analysis (C) of CD19-B16 tumor bearing animals treated with untransduced (control)(n=5), BBz CAR (n=5) or BBz CAR with CARD11-PIK3R3 (n=5) OT-1 T cells. P values determined using Log-rank (Mantel-Cox) test. * indicates P value<0.05, ** indicates P values<0.01.

FIG. 21 shows a schematic indicating the syngeneic CAR Mesothelioma Model used to assess CARD11-PIK3R3 therapeutic function in CAR T cells in vivo. hALPPL2-40Ls=hALPPL2-expressing tumor cells.

FIG. 22 shows tumor growth curves of hALPPL2 expressing 40L (mesothelioma) tumors injected subcutaneously and treated with untransduced, ALPPL2-BBz CAR or ALPPL2-BBz CAR with CARD11-PIK3R3 T cells. This data demonstrates that ALPPL2-BBz CAR T cells with CARD11-PIK3R3 expression induce some tumor control in the 40L model.

FIG. 23 is a schematic indicating the syngeneic TCR-transgenic Melanoma Model used to assess CARD11-PIK3R3 therapeutic function in T cells in vivo OT-I CD8+ T cells=T cells collected from CD45.1 C57BL/6-Tg(TcraTcrb)1100Mjb/J mice.

FIGS. 24A-B shows that CARD11-PIK3R3 OT-I cells preferentially expand and accumulate in tumors. It shows accumulation of TILs in the dual-transfer competition assay 7 days post T cell transfer. FIG. 24A demonstrates expansion via flow cytometry plots. FIG. 24B quantifies fold expansion of CARD11-PIK3R3 OT-Is in tumors in vivo over control. P values determined by ratio paired T test. **** indicates P value<0.0001.

FIG. 25 shows accumulation of CARD 11-PIK3R3 OT-I in tumor, spleen and draining lymph node 7 days post transfer of 1×10⁶ CARD11-PIK3R3 or control OT-Is. P values determined by unpaired T test. * indicates P value<0.05, **** indicates P value<0.0001.

FIG. 26A shows TCF-1 expression in OT-I+CARD11-PIK3R3 TILs. TCF-1 expression of TIL cells 7 days post transfer of 1×10⁶ CARD11-PIK3R3 or control OT-Is. FIG. 26B shows the frequency of TCF-1+OT-I cells in spleen and tumor draining lymph node (LN) of mice bearing B16-OVA subcutaneous tumors. P values determined by unpaired T test. ns indicates not significant, *** indicates P values<0.001.

FIG. 27 shows that CARD11-PIK3R3 OT-Is have improved functionality in vivo. It depicts ex vivo cytokine production of TILs isolated 7 days post transfer of 1×10⁶ CARD11-PIK3R3 or control OT-Is, and restimulated ex vivo with PMA/Ionomycin. P values determined by unpaired T test. * indicates P value<0.05, *** indicates P values<0.001.

FIGS. 28A and 28B show that CARD11-PIK3R3 OT-Is demonstrate improved anti-tumor efficacy in vivo. FIG. 28A depicts tumor size and FIG. 28B depicts survival analysis in B16-OVA melanoma bearing mice treated with PBS or OT-I cells (2×10⁶) 12 days after tumor inoculation. Complete response was defined as an absence of detectable tumor. **** indicates P value<0.0001.

FIGS. 29A and 29B show that CARD11-PIK3R3 OT-Is demonstrate improved functionality in vivo at low doses and during tumor rechallenge. Tumor size of B16-OVA melanoma bearing mice treated with OT-I CARD11-PIK3R3 T cells at 1×10⁵ (n=4) or 2×10⁵ (n=5) T cell dose as compared to 2×10⁶ control OT-I T cells (n=5) (A) and mice with cleared tumors from the dose response experiment rechallenged with B16-OVA melanoma tumors subcutaneously on the contralateral flank (B). *** indicates P values<0.001, **** indicates P value<0.0001.

FIGS. 30A-D shows results of experiments for assessing CARD11-PIK3R3 truncations in CD8+CD19-BBz CAR T cells in vitro. FIG. 30A is a schematic describing the CARD11-PIK3R3 coiled coil truncation designs, AID indicates removal of the inhibitory domain, −XX AA indicates the number of amino acids removed from the coiled coil region. FIG. 30B shows cell counts of CD19-BBz CAR CD8+ T cells with CARD11-PIK3R3 truncations over a period of 7 days after removal from anti-CD3/anti-CD28 bead stimulation. FIG. 30C shows CD19-BBz CAR CD8+ T cells with CARD11-PIK3R3 truncations co-cultured at 1:1 ratio with CD19 target cells for 24 hours, after which the supernatant was assessed via ELISA for IL-2 secretion. FIG. 30D show flow cytometry plots depicting CD19-BBz with CARD11-PIK3R3 or fusion variant CD8+ T cells and CD19-K562 populations after 14-day co-culture together without supplemental IL-2.

FIG. 31A depicts a lollipop diagram of STAT3 gene point mutations. FIG. 31B shows the in vivo screen log 2 fold change for each STAT mutation and wild-type as determined by MAGeCK, compared to baseline (pre-injection). Positive log 2 fold change indicates increased persistence in tumors compared to input.

FIG. 32A shows a lollipop diagram depicting BRAF gene point mutations. FIG. 32B shows CD19-BBz CAR Jurkat reporter activity Z score for NFAT, NF-κB, AP-1, and IL-2 for each BRAF mutation or wild-type after co-culture with CD19-K562s. Z score indicates the mean Z score of two independent biological replicates.

FIGS. 33A-B shows CARD1I mutations. FIG. 33A shows CD19-BBz CAR Jurkat reporter activity Z score for NFAT, NF-κB, AP-1 and IL-2 for each CARD11 mutation or wild-type after co-culture with CD19-K562s. Z score indicates the mean Z score of two independent biological replicates. FIG. 33B shows the in vivo screen log 2 fold change for each CARD11 mutation or wild-type as determined by MAGeCK, compared to baseline (pre-injection). Positive log 2 fold change indicates increased persistence in tumors compared to input.

FIG. 34A shows a lollipop diagram depicting RASGRP1 gene point mutations. FIG. 34B CD19-BBz CAR Jurkat reporter activity Z score for NFAT, NF-κB, AP-1 and IL-2 for each RASGRP1 mutation or wild-type after co-culture with CD19-K562s. Z score indicates the mean Z score of two independent biological replicates. FIG. 34C shows the in vivo screen log 2 fold change for each RASGRP1 mutation or wild-type as determined by MAGeCK, compared to baseline (pre-injection). Positive log 2 fold change indicates increased persistence in tumors compared to input.

FIG. 35A depicts a lollipop diagram showing various PLCG1 gene point mutations. FIG. 35B is a graph showing CD19-BBz CAR Jurkat reporter activity Z score for NFAT, NF-κB, AP-1 and IL-2 for each PLCG1 mutation after co-culture with CD19-K562s. Z score indicates the mean Z score of two independent biological replicates.

FIG. 36A is a lollipop diagram depicting TNFRSF1B gene point mutations. FIG. 36B shows a bar graph illustrating CD19-BBz CAR Jurkat reporter activity Z score for NFAT, NF-κB, AP-1 and IL-2 for each TNFRSF1B mutation or wild-type after co-culture with CD19-K562s. Z score indicates the mean Z score of two independent biological replicates. FIG. 36C shows the in vivo screen log 2 fold change for each TNFRSF1B mutation or wild-type as determined by MAGeCK, compared to baseline (pre-injection). Positive log 2 fold change indicates increased persistence in tumors compared to input.

FIG. 37A is a lollipop diagram depicting JAK/JAK3 gene mutations. FIG. 37B shows the in vivo screen log 2 fold change for each JAK1/JAK3 mutation as determined by MAGeCK, compared to baseline (pre-injection). Positive log 2 fold change indicates increased persistence in tumors compared to input.

FIG. 38 shows NF-κB reporter activity of control, CARD11, or BCL10 CRISPR knockout BBz-CAR Jurkat cells.

FIG. 39A shows Principal component analysis of transcriptomes of human BBz-CAR T cells. FIG. 39B is a heatmap of significantly upregulated genes in CARD11-PIK3R3 compared to control BBz-CAR T cells after stimulation with CD19 antigen shared among both CD4+ and CD8+ T cells. FIG. 39C shows the top 5 Reactome pathways enriched in CD4+ and CD8+ T cells (top) and NF-κB, AP-1, and MALT1 gene signature enrichment in CARD11-PIK3R3 expressing CD8+ BBz-CAR T cells after stimulation (bottom).

FIG. 40 shows activation markers expressed on transduced CD4 and CD8 T cells 24 hours post 1:1 co-culture with CD19-K562s. Ratio of MFI in CARD11-PIK3R3 relative to control shown. P values determined by unpaired T test.

FIG. 41 shows cytokine secretion of CD4+CD19-BBz-CAR T cells and CD19-BBz-CAR+CARD11-PIK3R3 T cells after 48 hours of stimulation.

FIG. 42A shows control (n=6), CD19-BBz-CAR (n=7), CD19-BBz-CAR+CARD11-PIK3R3 (n=7) dosed at 7e6 CAR+ T cells. CARD11-PIK3R3 T cell (n=7) dose was equivalent to the total number of CARD11-PIK3R3 expressing T cells dosed in the CD19-BBz-CAR+CARD11-PIK3R3 group. FIG. 42B shows control (n=7), CD19-CD28z-CAR (n=7), CD19-CD28z-CAR+CARD11-PIK3R3 (n=7) T cells dosed at 4e5 CAR+ T cells. FIG. 42C shows survival analysis of part (b) surviving CD19-CD28z-CAR+CARD11-PIK3R3 (n=6) animals or naive mice (n=4) rechallenged with 5e5 Nalm6-Luc-GFP tumors. FIG. 42D shows tumor volumes of M28 tumor bearing animals treated with control (n=5), MCAM-CD28z-CAR (n=5), or MCAM-CD28z-CAR+CARD11-PIK3R3 (n=5). T cells dosed at 5e5 CAR+ cells, control cells dosed equivalent to highest total T cell dose in other treatment groups.

FIG. 43A shows flow cytometry plots indicating CAR (FLAG) and CARD11-PIK3R3 (mCherry) expression in human CD3 T cells. FIG. 43B and FIG. 43C show percent weight change from baseline of (FIG. 43B) tumor bearing or (FIG. 43C) non-tumor bearing animals treated with control, CD19-BBz-CAR, CD19-BBz-CAR+CARD11-PIK3R3 or CARD11-PIK3R3. FIG. 43D shows survival analysis of FIG. 42B surviving CD19-BBz-CAR (n=3), CD19-BBz-CAR+CARD11-PIK3R3 (n=4) animals or naive mice (n=4) rechallenged with 5e5 Nalm6-Luc-GFP tumors. P values determined by Log-rank Mantel-Cox. * indicates P value<0.05.

FIG. 44A and FIG. 44B show flow cytometry plots indicating FLAG (CAR) and mCherry (CARD11-PIK3R3) expression in human CD3 T cells. FIG. 44C shows Percent weight change from baseline of control, MCAM-CD28z-CAR or MCAM-CD28z-CAR+CARD11-PIK3R3 treated M28 tumor bearing animals.

FIG. 45 shows tumor size of SNU-1 HLA-C*08:02 gastric cancer xenograft bearing mice treated with control (n=5), KRAS p.G12D-specific TCR T cells (n=5), or KRAS p.G12D-specific TCRCARD11-PIK3R3 T cells (n=6). Complete response was defined as an absence of a detectable tumor. * indicates P value<0.05, *** indicates P values<0.001, **** indicates P value<0.0001.

FIG. 46A, FIG. 46B, and FIG. 46C show in vitro expansion of CD19-BBz-CAR T cells with and without CARD11-PIK3R3. FIG. 46A and FIG. 46B show CAR or CAR+CARD11-PIK3R3 were sorted for purity, then expanded with IL-2 for 12 days. On Day 12,

cultures were split, reseeding each group without IL-2 (FIG. 46A) and with IL-2 (FIG. 46B). Cells were counted and split from day 12 to day 30. FIG. 46C shows, on Day 30, CD19-BBz-CAR+CARD11-PIK3R3 T cells that were cultured with IL-2 were reseeded without IL-2 and counted and split for an additional 10 days.

FIG. 47 shows in vivo analysis of CARD11-PIK3R3 expressing CAR T cells. Histograms are shown indicating human CD19 ligand expression on hCD19-B16 tumors, CD19-BBz-CAR or CD19-BBz-CAR+CARD11-PIK3R3 treated with that had reach euthanasia endpoint, compared to known CD19 positive B16 tumor sample.

FIG. 48A, FIG. 48B, and FIG. 48C show in vivo analysis of CARD11-PIK3R3 expressing OT-I T cells. FIG. 48A shows a schematic of competition experiments with CARD11-PIK3R3 and CARD11-PIK3R3 R28A pmel-1 CD8 T cells in B16-F10 tumor model. FIG. 48B shows tumor growth curves of mice described in (a). FIG. 48C shows fold-enrichment in the tumor of wild-type CARD11-PIK3R3 or CARD11-PIK3R3 (p.R28A) expressing pmel-1 CD8 T cells compared to vector control 7 days after adoptive transfer. All P values determined by ratio paired T test.

FIGS. 49A-49J show long-term evaluation of B6 mice treated with CARD11-PIK3R3 expressing OT-I T cells. FIG. 49A shows mice that cleared B16-F10-OVA were monitored for up to 240 days after adoptive T cell transfer. Necropsies were performed in as outlined in this schematic. FIG. 49B shows body weights of all CARD11-PIK3R3 infused mice from FIGS. 29A-29B were measured weekly and compared to expected weight curves published by The Jackson Laboratory Research Institute. FIG. 49C shows spleen weight for three animals that underwent necropsy on day 240. This was calculated as percent of body weight and compared to expected spleen weight published by The Jackson Laboratory Research Institute FIG. 49D shows necropsy of one representative animal. None of the three animals had gross pathology.

FIG. 49E shows the Percentage of CD8 T cells in spleen and blood that express CARD11-PIK3R3 240 days after adoptive transfer. FIG. 49F shows representative hematoxylin and eosin-stained tissue sections from select organs where nodal or extranodal lymphomas can occur. Animals were subject to full-body necropsies. Tissues did not show evidence of nuclear atypia, changes in cellular architecture, or presence of neoplastic disease. Representative images at low (left) and high (right)-power magnification are shown with size bars. White box reflects the site of high-magnification image. FIG. 49G and FIG. 491 show a schematic of blood sampling of mice presented in FIGS. 29A-29B 330 days after adoptive transfer (FIG. 49G) or of 2e6 OT-I CARD11-PIK3R3 treated mice 418 days after adoptive transfer (FIG. 49I). FIG. 49H and FIG. 49J shows the percentage of CD8 T cells in blood that express CARD11-PIK3R3.

In the following detailed description, reference is made to the Figures, which form a part hereof. In the Figures, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

### DETAILED DESCRIPTION OF THE DISCLOSURE

### Overview

In general, the present disclosure relates, inter alia, to compositions and methods for improving adoptive T cell therapy. The disclosers have discovered ways to improve the therapeutic efficacy of T cells by altering T cell signaling, decreasing T cell exhaustion and/or by enhancing in vivo persistence and fitness of engineered T cells. T cell cancers can undergo positive selection of beneficial genetic alterations (e.g., mutations). In some embodiments, the present disclosure relates to exploiting such beneficial mutations to improve the effectiveness of T cell therapeutics.

The disclosure also relates to polypeptides having one or more mutations that can alter or promote or enhance signaling in T cells. The disclosure also relates to recombinant nucleic acid constructs and/or recombinant nucleic acids encoding polypeptides having one or more mutations that can alter or promote or enhance signaling in T cells. Non-limiting examples of such mutations are shown in Table 1. Non-limiting examples of signaling in T cells include: 1) CARD11-BCL10-MALT1 complex signaling, 2) JAK/STAT signaling, 3) co-stimulatory molecule signaling, 4) RAS/MEK/ERK signaling, 5) phospholipase gamma signaling, 6) transcription factor activity and/or other signaling pathways.

In some embodiments, the mutation alters a TCR transcriptional signaling output. In some embodiments, the mutation inhibits or reduces a TCR transcriptional signaling output e.g., NFAT, NF-κB or AP-1 signaling) or cytokine outputs (e.g., IL-2)

The disclosure also relates to polypeptides having one or more mutations, that can reduce T cell exhaustion, increase proliferation, alter effector function, resist T cell dysfunction, increase T cell fitness, enhance in vivo persistence, and/or increase intratumoral presence of therapeutic T cells. The disclosure further relates to recombinant nucleic acid constructs and/or recombinant nucleic acids encoding polypeptides having one or more mutations, that can reduce T cell exhaustion, increase proliferation, alter effector function, resist T cell dysfunction, increase T cell fitness, enhance in vivo persistence, and/or increase intratumoral presence of therapeutic T cells.

Reprograming T cells for proliferation, cytokine production and differentiation towards effector cells after engagement of a T cell receptor complex can depend on activating costimulatory signals and can be counteracted by coinhibitory molecules. The transcription factors NF-κB, NFAT and AP-1 have a major role in inducing the transcriptional program that is required for T cell activation and differentiation. Measuring the expression of such transcription factors are within the methods of the current disclosure and can be indicative to the major changes in T cell function for the mutations of the disclosure.

### Definitions

Unless otherwise defined, all terms of art, notations, and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art. All publications, patent applications, patents, GenBank or other accession numbers and other references mentioned herein are incorporated by reference in their entirety for all purposes.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B.”

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As will be understood by one having ordinary skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It is understood that aspects and embodiments of the disclosure described herein include “comprising,” “consisting,” and “consisting essentially of” aspects and embodiments. As used herein, “comprising” is synonymous with “including,” “containing,” or “characterized by,” and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or step.

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI web site at ncbi.nlm.nih.gov/BLAST. Such sequences are then the to be “substantially identical.” This definition also refers to, or may be applied to, the complement of a test sequence. This definition also includes sequences that have deletions and/or additions, as well as those that have substitutions. Generally, sequence identity can exist over a region that is at least about 20 amino acids or nucleotides in length, or over a region that is 10-100 amino acids or nucleotides in length, or over the entire length of a given sequence

If necessary, sequence identity can be calculated using published techniques and widely available computer programs, such as the GCS program package (Devereux et al, Nucleic Acids Res. 12:387, 1984), BLASTP, BLASTN, FASTA (Atschul et al., J. Molecular Biol. 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

As used herein, the term “mutation” refers to a point mutation, a gene fusion, a substitution, a gain-of-function mutation, a stop-gain mutation, an insertion mutation, a deletion mutation, a duplication mutation and/or a translocation. The mutation may be in one or more genes. The mutation may be naturally occurring. Alternatively, the mutation may be induced or engineered. As used herein, the term “vector” refers to a recombinant polynucleotide construct designed for transfer between host cells, and that may be used for the purpose of transformation, e.g., the introduction of heterologous DNA into a host cell. As such, in some embodiments, the vector can be a replicon, such as a plasmid, phage, or cosmid, into which another DNA segment may be inserted so as to bring about the replication of the inserted segment. In some embodiments, the expression vector can be an integrating vector.

As used herein, the term “viral vector” refers either to a nucleic acid molecule (e.g., a transfer plasmid) that includes virus-derived nucleic acid elements that generally facilitate transfer of the nucleic acid molecule or integration into the genome of a cell or to a viral particle that mediates nucleic acid transfer. Viral particles will generally include various viral components and sometimes also host cell components in addition to nucleic acid(s). The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. Viral vectors that can be used in the disclosure include, for example, retrovirus vectors, adenovirus vectors, and adeno-associated virus vectors, lentivirus vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.). For example, a recombinant polypeptide as disclosed herein can be produced in a eukaryotic host, such as a mammalian cells (e.g., COS cells, NIH 3T3 cells, or HeLa cells). These cells are available from many sources, including the American Type Culture Collection (Manassas, VA). In selecting an expression system, care should be taken to ensure that the components are compatible with one another. Artisans or ordinary skill are able to make such a determination. Furthermore, if guidance is required in selecting an expression system, skilled artisans may consult P. Jones, “Vectors: Cloning Applications”, John Wiley and Sons, New York, N.Y., 2009).

As used herein, the term “retroviral vector” refers to a viral vector or plasmid containing structural and functional genetic elements, or portions thereof, that are primarily derived from a retrovirus. The retroviral vector can be a lentiviral vector. As used herein, the term “lentiviral vector” refers to a viral vector or plasmid containing structural and functional genetic elements, or portions thereof, including LTRs that are primarily derived from a lentivirus, which is a genus of retrovirus. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile; and (vii) a relatively easy system for vector manipulation and production.

As used herein, the term “pharmaceutically acceptable carrier” as used herein means any suitable carriers, diluents or excipients. These include all aqueous and non-aqueous isotonic sterile injection solutions, which may contain anti-oxidants, buffers and solutes, which render the composition isotonic with the blood of the intended recipient; aqueous and non-aqueous sterile suspensions, which may include suspending agents and thickening agents, dispersion media, antifungal and antibacterial agents, isotonic and absorption agents and the like. It will be understood that compositions of the present disclosure may also include other supplementary physiologically active agents. The carrier must be pharmaceutically “acceptable” in the sense of being compatible with the other ingredients of the composition and not injurious to the subject.

As used herein, the term “PEGylation” refers to modifying a protein by covalently attaching polyethylene glycol (PEG) to the protein, with “PEGylated” referring to a protein having a PEG attached. A range of PEG, or PEG derivative sizes with optional ranges of from about 10,000 Daltons to about 40,000 Daltons may be attached to the recombinant polypeptides of the disclosure using a variety of chemistries. In some embodiments, the average molecular weight of the PEG, or PEG derivative, is about 1 kD to about 200 kD such as, e.g., about 10 kD to about 150 kD, about 50 kD to about 100 kD, about 5 kD to about 100 kD, about 20 kD to about 80 kD, about 30 kD to about 70 kD, about 40 kD to about 60 kD, about 50 kD to about 100 kD, about 100 kD to about 200 kD, or about 1 150 kD to about 200 kD. In some embodiments, the average molecular weight of the PEG, or PEG derivative, is about 5 kD, about 10 kD, about 20 kD, about 30 kD, about 40 kD, about 50 kD, about 60 kD, about 70 kD, or about 80 kD. In some embodiments, the average molecular weight of the PEG, or PEG derivative, is about 40 kD.

As used herein, the terms “administration” and “administering” refer to the delivery of a bioactive composition or formulation by an administration route including, but not limited to, oral, intravenous, intra-arterial, intramuscular, intraperitoneal, subcutaneous, intramuscular, and topical administration, or combinations thereof. The term includes, but is not limited to, administering by a medical professional and self-administering.

As used herein, the term “injection” includes intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion.

The term “cancer” generally refers to the presence of cells possessing characteristics typical of cancer-causing cells, such as uncontrolled proliferation, immortality, metastatic potential, rapid growth and proliferation rate, and certain characteristic morphological features. Cancer cells can be in the form of a tumor, but such cells can exist alone within an animal subject, or can be a non-tumorigenic cancer cell, such as a leukemia cell. These terms include a solid tumor, a soft tissue tumor, or a metastatic lesion. As used herein, the term “cancer” includes premalignant, as well as malignant cancers. In some embodiments, the cancer is a solid tumor, a soft tissue tumor, or a metastatic lesion.

As used herein, and unless otherwise specified, a “therapeutically effective” or “pharmaceutically effective” amount or number of a subject construct, nucleic acid, cell, or composition of the disclosure generally refer to an amount or number sufficient for a construct, nucleic acid, cell, or composition to accomplish a stated purpose relative to the absence of the composition, e.g., to provide a therapeutic benefit in the treatment or management of the cancer, or to delay or minimize one or more symptoms associated with the cancer. A therapeutically effective amount of a compound means an amount of therapeutic agent, alone or in combination with other therapeutic agents, which provides a therapeutic benefit in the treatment or management of the cancer. The term “therapeutically effective amount” can encompass an amount that improves overall therapy, reduces or avoids symptoms or causes of the cancer, or enhances the therapeutic efficacy of another therapeutic agent. An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins)

As used herein, a “subject” or an “individual” includes animals, such as human (e.g., human subjects) and non-human animals. In some embodiments, a “subject” or “individual” is a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, e.g. non-human primates, and non-mammals, e.g., sheep, dogs, cows, chickens, amphibians, reptiles, etc.

Headings, e.g., (a), (b), (i) etc., are presented merely for ease of reading the specification and claims. The use of headings in the specification or claims does not require the steps or elements be performed in alphabetical or numerical order or the order in which they are presented. It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### TCR Signaling

Upon engagement of a peptide-MHC complex, the immunoreceptor tyrosine-based activation motifs (ITAMs) contained within the intracellular tails of the TCR complex become phosphorylated and recruit Zap70. Zap70 then recruits and activates the downstream LAT signalosome containing LAT, PLCy, Slp76. This signalosome causes the activation of the Ras/MAPK/ERK signaling leading to AP-1 transcriptional activity, the activation of PKCθ leading to NF-κB transcriptional activity and finally the influx of calcium which is responsible for NFAT transcriptional activity. Additionally, signaling from costimulatory receptors, such as 41BB, and cytokine support from IL-2 enhances these signaling pathways, and is required for T cells to undergo full activation and avoid anergic cell death. When fully activated, T cells rapidly proliferate and differentiate, secrete inflammatory cytokines, and cytotoxic cells begin performing target killing through release of cytotoxic granules. After resolution of this acute response, activated T cells undergo contraction to a form a small population of long-lived memory T cells that surveil the body, ready to respond to future encounters with the antigen.

### T Cell Signaling

T cells become fully activated through T cell receptor (TCR) engagement, costimulatory signaling and cytokine support. TCR activation induces NFAT, NF-κB and AP-1 transcription factors signaling, IL-2 and other inflammatory cytokine secretion.

### T Cell Exhaustion

T cell exhaustion is a state of hypo-responsiveness induced in effector T cell populations after chronic exposure to antigen. Exhaustion is marked by the upregulation of surface inhibitory receptors such as PD-1, TIM-3, LAG-3 and CTLA-4 among others, and the inability to proliferate long term, secrete inflammatory cytokines and kill target cells effectively. Exhausted T cells exist in a distinct transcriptional and epigenetic landscape. While exhaustion has been well characterized in chronic viral infections, it is also implicated in cancer, in which tumor infiltrating T cells, and engineered T cell therapies exhibit signs of exhaustion and fail to control tumor growth. To better treat chronic infections or cancer, T cell exhaustion must be prevented or overcome. In fact, checkpoint therapies which block signaling from the PD-1 or CTLA-4 axis of inhibition have proven somewhat effective in reinvigorating T cell responses in certain cancers leading to limited but effective tumor control.

### NFAT Signaling:

The transcription factor nuclear factor of activated T cells (NFAT) is involved in T cell activation as well as programs of T cell exhaustion. At resting state, NFAT transcription factors are found in the cytoplasm in a phosphorylated state, unable to translocate to the nucleus to induce signaling. The extracellular influx of Ca²⁺ that occurs during T cell activation activates the calcium dependent phosphatase calcineurin, resulting in dephosphorylation of NFAT, and its subsequent translocation and signaling in the nucleus. NFAT binds DNA in multiple ways, as a monomer, dimer or as a complex with other transcription factors. In particular NFAT is known to cooperatively bind with the transcription factor AP-1, inducing transcription of activation associated genes critical for effector T cell function. In contrast, when NFAT is not bound by AP-1, but is instead partnerless, it has been found to induce the expression of exhaustion associated genes, such as the inhibitory receptor PD-1. Additionally, studies have determined that exhaustion induced by partnerless NFAT can be remedied in CAR T cells by over-expressing the AP-1 family member, cJun. NFAT induces the expression of transcription factors TOX and NR4A1/2/3, which have been associated with T cell exhaustion and deletion or knockdown of these transcription factors has improved T cell phenotype and in vivo tumor control.

As NFAT signaling is key to successful T cell activation, but also plays a role in exhaustion, particularly in the absence of AP-1, it can be used to determine how genetic modifications in T cells influences the dynamics of NFAT signaling. Targeting the NFAT transcriptional pathway can be beneficial for T cell therapeutics.

### NF-κB Signaling:

NF-κB (nuclear factor kappa light chain enhancer of B cells) is a family of transcription factors that induces transcriptional programs critical for T cell activation and effector function. NF-κB signaling occurs in many cell types. In T cells, NF-κB signaling induces a wide range of transcriptional programs responsible for proliferation and memory formation, resistance to apoptosis, cytokine secretion, and the production of a robust effector T cell response. NF-κB signaling is induced through two pathways, the canonical and noncanonical pathways.

In the canonical pathway NFKB1 is bound in the cytoplasm by IkBa and IkB-like molecule p105, forming a complex that prevents the nuclear translocation of NKFB1. When activated, TCR signaling induces activation of PKCθ, which in turn phosphorylates and activates CARD11. Activated CARD11 forms a complex with BCL10 and MALT1, and ultimately causes the phosphorylation and degradation of IkBa and IkB-like molecule p105, releasing NF-κB to translocate to the nucleus.

The non-canonical pathway is triggered by signaling through tumor necrosis factor receptor (TNFR) family members, which includes the costimulatory domain 41BB. At rest, the NFKB2 is bound by p100, during activation NF-κB-inducing kinase (NIK) activates IKKa, which in turn phosphorylates p100 and leads to the release and translocation of NKFB2 to the nucleus.

In syngeneic models of solid tumors, endogenous T cells that can respond to the tumors required NF-κB signaling to mediate tumor clearance. While NF-κB is induced through TCR signaling, it is also stimulated through the costimulatory domain 41BB (a TNFRS superfamily member), which is used clinically in FDA approved chimeric antigen receptor (CARs) therapies. CAR therapies using 41BB costimulatory domains have been found to persist longer in patients as compared to CAR therapies using other costimulatory domains, such as CD28. The persistence of 41BB CARs is directly linked to NF-κB signaling, which improves CAR T resistance to apoptosis through suppression of apoptotic proteins such as Bim.

### AP-1 Signaling:

AP-1 transcription factors are a family of homo or hetero-dimeric proteins formed from complexes of JUN, FOS, ATF or MAF proteins. AP-1 signaling is induced by the phosphorylation cascade called the Mitogen Activated Protein Kinase (MAPK) pathway, triggered by TCR, cytokine/chemokine, or growth factor signaling. AP-1 often signals in complex with NFAT, and as indicated in the NFAT section, the loss of AP-1 causes partnerless NFAT to induce exhaustion programs in T cells, however this exhaustion can be remedied by overexpression of the AP-1 family member c-Jun. Related to this, loss of AP-1 is also known to induce an anergic cell state, where the T cell has partially activated through the TCR, but does not have enough costimulatory and cytokine signaling to induce full activation.

AP-1 dimers are activated by a plethora of physiological and pathological stimuli. Studies have reported that AP-1 proteins, mostly those that belong to the Jun group, control cell life and death through their ability to regulate the expression and function of cell cycle regulators such as Cyclin D1, p53, p21(cip1/waf1), p19(ARF) and p16. Amongst the June proteins, c-Jun is unique in its ability to positively regulate cell proliferation through the repression of tumor suppressor gene expression and function, and induction of cyclin D1 transcription. These actions are antagonized by JunB, which upregulates tumor suppressor genes and represses cyclin D1. An important target for AP-1 effects on cell life and death is the tumor suppressor p53, whose expression as well as transcriptional activity, are modulated by AP-1 proteins.

The concomitant induction of NFAT and AP-1 takes concerted activation of two different signaling pathways: calcium/calcineurin, which promotes NFAT dephosphorylation, nuclear translocation and activation; and protein kinase C (PKC)/Ras, which promotes the synthesis, phosphorylation, and activation of members of the Fos and Jun families of transcription factors. (Shaulian E, Karin M. AP-1 as a regulator of cell life and death. Nat Cell Biol 2002 45. 2002; 4(5):E131-E136.)

### IL-2 Signaling:

IL-2 is a pleiotropic cytokine required for activation, proliferation differentiation, and maintenance of T cells. Naïve T cells express a low affinity IL-2 receptor requiring high volumes of IL-2 to initiate activation, while memory and regulatory T cells express a high affinity IL-2 receptor requiring much lower amounts of IL-2 for effective signaling. The IL-2 receptor makes use of the JAK/STAT signaling cascade, resulting in wide ranging transcriptional changes. Though CD8+ T cells are responsible for the cytotoxic effector response to foreign antigens, CD8+ T cells themselves cannot effectively produce IL-2 and rely instead on helper CD4+ T cells to produce IL-2 and other cytokine support.

IL-2 is so critical for T cell persistence and proliferation that high dose IL-2 therapy has been FDA approved for certain cancers, with the hopes of expanding endogenous cytotoxic T cell populations to induce tumor rejection. In metastatic melanoma and renal cell carcinoma trials, high dose IL-2 therapy induces limited (7%) long term response rates.

However this therapy is not well tolerated by patients, causing multiple deaths, and likely contributes to the expansion of CD4+CD25+FoxP3+ regulatory T cells. Regulatory T cells are known to be pro-tumorigenic and high numbers of regulatory T cells (Tregs) are correlated with poor prognosis in solid tumor settings. Toxicity and the expansion of pro-tumor Treg populations present so much of a challenge to IL-2 therapy that synthetic orthogonal IL-2 cytokine therapies have recently been developed to target the effects of IL-2 cytokine therapy to cytotoxic T cells and avoid expansion of Tregs.

IL-2 secretion is highly relevant for the persistence and proliferation of T cell therapeutic products, therefore the inventors/disclosers assessed IL-2 secretion capacity of each mutation when expressed in CAR Jurkat cell lines and after co-culture with target cells. As a follow up for relevant hits, the inventors/disclosers assessed the capacity for mutations to improve CAR T cell in vitro target killing when cultured without supplemental IL-2. This setting, where IL-2 is withheld, better reflects the challenges of the tumor microenvironment, where IL-2 is scarce and autocrine secretion of IL-2 would be beneficial for maintenance of proliferation and long-term killing capabilities.

### In Vivo Screening of Mutations:

A major barrier to the success of cell therapy is the accumulation and persistence of T cells within tumors. The inventors/disclosers sought to systematically screen mutations in primary human CAR T cells in a xenograft model. To uncover mutations that improve persistence in highly adverse conditions, the inventors/disclosers utilized a difficult to control K562 subcutaneous tumor model in which T cell efficacy is limited.

The inventors/disclosers found that clinically relevant CD19 targeted BBz CAR T cells are ineffective in controlling CD19-K562 tumors in a subcutaneous xenograft model, likely failing due to low CAR T cell infiltration, and the lack of persistence and expansion within the tumor microenvironment. These challenges mirror some of the CAR T cell clinical failures observed when treating solid tumors. The inventors/disclosers opted to screen the mutations in this high bar “failure” model with the hopes of finding mutations that greatly improve CAR T cell persistence within solid tumors.

While adoptive cell therapies have proven highly effective in refractory B cell malignancies, CAR-T cell therapies have yet to provide robust, long-term efficacy against solid tumors. In the solid tumor setting, CAR-T cells become exhausted and struggle to proliferate and perform effector function, ultimately resulting in the inability to control tumor growth or prevent relapse. Therefore, the inventors/disclosers opted to create effective targeted cellular therapies against solid tumors by improving the proliferative capacity, persistence, and effector function of CAR-T cells.

### CARD11-BCL10-Malt Signalosome

Formation of a CARD11-BCL10-MALT (CBM) signaling complex is a key event in T- and B cell receptor-induced gene expression. After exposure to distinct immune triggers, these molecules form self-organizing filaments with MALT1 protease activity to regulate canonical nuclear factor-κB (NF-κB) and mitogen-activated protein kinase (MAPK) signalling pathways and the degradation of mRNA-binding proteins, which provides two layers of control of inflammatory gene expression. Deregulation of CARD11, BCL10 or MALT1 expression or CBM signaling have been associated with cancer, immunodeficiency, and autoimmunity (J. Ruland, L. Hartjes, CARD-BCL-10-MALT1 signalling in protective and pathological immunity. Nat Rev Immunol 19, 118-134 (2019).

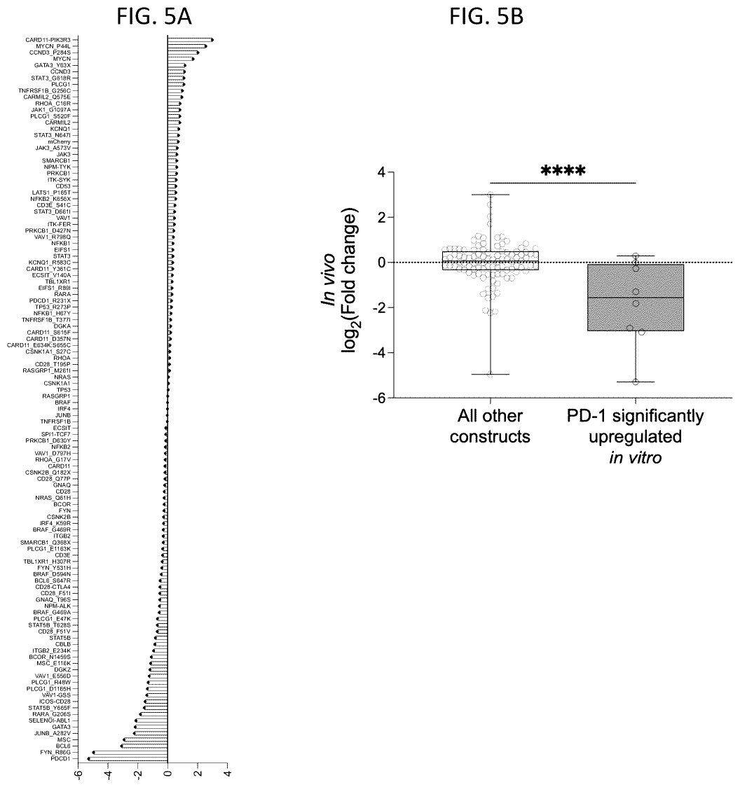

In normal T cells, T cell receptor (TCR) signaling activates PKCθ, which in turn promotes the assembly of the CARD11-BCL10-MALT1 (CBM) signalosome. The CBM complex subsequently has three major outputs: NF-κB transcriptional activity, AP-1 transcriptional activity and MALT1 proteolytic activity (FIG. 5). The assembly of the CARD11-BCL10-MALT1 signalosome complex is an essential step in regulating NF-κB in lymphoid immune cells.

An inhibitory domain present in CARD11 may allow for intramolecular autoinhibition which can prevent CARD11 binding to BCL10 in the absence of upstream signals. Upon phosphorylation of the inhibitory domain in normal T cells, CARD11 autoinhibition can be relieved and the CARD11 protein can oligomerize and promote prion-like assembly of the CBM complex with recruitment of BCL10-MALT1 filaments, which then can allow for CBM complex signaling.

Genes involved in CBM signaling include but are not limited to caspase recruitment domain family member 11 (CARD11), capping protein regulator and myosin 1 linker 2 (CARMIL2), mucosa-associated lymphoid tissue lymphoma translocation protein 1 (MALT1), B-cell lymphoma 10 (BCL10). CARD11 can be involved in both the innate and adaptive immune systems. CARD11 is implicated in the activation of NF-kB by the TCR complex.

### JAK/STAT Signaling

The biochemistry of JAK/STAT signaling is well known to a person of skill in the art. Briefly, signaling begins with extracellular association of cytokines or growth factors with their corresponding transmembrane receptors. This facilitates trans-activation of receptor-bound Janus kinases (JAKS) by putting them in spatial proximity and by prompting conformational changes that distance their kinase domains from inhibitory pseudokinase domains. Activated JAKS then phosphorylate latent STAT monomers, leading to dimerization, nuclear translocation, and DNA binding. In mammals, 4 JAKS (JAK1, JAK2, JAK3, TYK2) and 7 STATs (STAT1, STAT2, STAT3, STAT4, STAT5a, STAT5b, and STAT6) are employed by more than 50 cytokines and growth factors. (Villarino A V., Kanno Y, O'Shea J J. Mechanisms and consequences of JAK-STAT signaling in the immune system. Nat Immunol 2017 184. 2017; 18(4):374-384).

### Co-Stimulatory Molecule Signaling

Co-stimulatory and co-inhibitory molecules are cell surface receptors and ligands that are classified into various families on the basis of their structure and functions.

Co-stimulatory and co-inhibitory receptors determine the functional outcome of T cell receptor (TCR) signaling. The specific recognition of cognate antigenic peptides presented by MHC molecules triggers T cell receptor signaling, but it is co-stimulatory and co-inhibitory receptors on T cells that direct T cell function and determine T cell fate. T cell co-signaling receptors have been broadly defined as cell-surface molecules that can transduce signals into T cells to positively (co-stimulatory receptors) or negatively (co-inhibitory receptors) modulate TCR signaling. Examples of co-stimulatory or co-inhibitory receptors includes CD28 and CTLA-4, both of which bind to ligands B7-1 and 7-2. Other genes involved in co-stimulatory molecule signaling include TNFR2, TNFRS1B, and ICOS (Chen L, Flies D B. Molecular mechanisms of T cell co-stimulation and co-inhibition. Nat Rev Immunol 2013 134. 2013; 13(4):227-242.) RAS/MEK/ERK

As is known by the skilled in the art, the sarcoma/mitogen-activated protein kinase kinase/extracellular receptor kinase (RAS/MEK/ERK) is a conserved signaling pathway that plays pivotal roles in cell proliferation, survival and differentiation. The aberrant activation of the RAS/MEK/ERK signaling pathway induces tumors. Efforts have been dedicated to targeting this signaling pathway for cancer treatment. The aberrant activation of the signaling pathway contributes to tumorigenesis and tumor development.

It is also known that triggering of a T-cell receptor by its cognate antigen results in nearly immediate activation of downstream signaling cascades, including the RAS/MEK/ERK pathway. Studies have also shown that RAS/MEK/ERK signaling is memory stage-dependent in human T cells, conferring susceptibility to alloreactive T-cell selective inhibition.

As an important upstream molecular marker of the RAS-RAF-MEK-ERK pathway, RAS serves as a molecular switch by binding GTP/GDP, and it includes four isoforms:HRAS, KRAS4A, KRAS4B, and NRAS. KRAS is the most frequent isoform in all human cancers. KRAS4A and KRAS4B are the different splicing isoforms of the same gene. When the transmembrane receptors (receptor tyrosine kinase, RTKs) are activated, the complexes of growth-factor-receptor bound protein 2 (GRB2) and son of sevenless (SOS) in the cytoplasm are recruited to the inner surface of cell membrane. (McCubrey J A, Steelman L S, Bäsecke J, Martelli A M. Raf/mek/erk signaling. Target Ther Acute Myeloid Leuk. January 2015:275-305.)

Several mutations identified by the inventors/disclosers are in genes having various roles in this signaling pathway.

### Phospholipase C Gamma Signaling

Phospholipase C (PLC) is an essential mediator of cellular signaling. PLC regulates multiple cellular processes by generating bioactive molecules such as inositol-1,4,5-triphosphate (IP3) and diacylglycerol (DAG). These products propagate and regulate cellular signaling via calcium (Ca2+) mobilization and activation of protein kinase C (PKC), other kinases, and ion channels. PLCγ1, one of the primary subtypes of PLC, is directly activated by membrane receptors, including receptor tyrosine kinases (RTKs), and adhesion receptors such as integrin. PLCγ1 mediates signaling through direct interactions with other signaling molecules via SH domains, as well as its lipase activity. PLCγ1 is frequently enriched and mutated in various cancers, and is involved in the processes of tumorigenesis, including proliferation, migration, and invasion. (Jang H J, Suh P G, Lee Y J, Shin K J, Cocco L, Chae Y C. PLCγ1: Potential arbitrator of cancer progression. Adv Biol Regul. 2018; 67:179-189, and Patterson R L, Van Rossum D B, Nikolaidis N, Gill D L, Snyder S H. Phospholipase C-y: diverse roles in receptor-mediated calcium signaling. Trends Biochem Sci. 2005; 30(12):688-697

The phospholipase C gamma signaling pathway has been implicated in T cell lymphomas and primarily in cutaneous T cell lymphomas (CTCL). Nine PLCG1 mutations (p.R48W, p.S312L, p.D342N, p.S345F, p.S520F, p.R1158H, p.E1163K, p.D1165H, and the in-frame indel p.VYEEDM1161V) have been identified in Sézary Syndrome, the leukemic variant of CTCL. (V. M. Patel et al., Frequent and Persistent PLCG1 Mutations in Sézary Cells Directly Enhance PLCγ1 Activity and Stimulate NFκB, AP-1, and NFAT Signaling. J Invest Dermatol 140, 380-389.e384 (2020).

Recombinant Nucleic Acid Constructs and/or Recombinant Nucleic Acids and Polypeptides

Certain aspects of the disclosure are directed to polypeptides, recombinant nucleic acid constructs, and recombinant nucleic acids encoding polypeptides, wherein the polypeptides include a mutation capable of altering T cell signaling.

Exemplary mutations in the polypeptides encoded by the recombinant nucleic acid constructs and/or recombinant nucleic acids of the disclosure are described in Table 1. In some embodiments of the recombinant nucleic constructs and/or recombinant nucleic acids of the disclosure, the polypeptides can include more than one mutation. For example, one or two or three or four or five or more mutations. In some embodiments, the mutations are a combination of different types of mutations. The different types of mutations can be point mutations, gene fusions, substitutions, gain-of-function mutations, stop-gain mutations, insertion mutations, deletion mutations, duplication mutations or translocations. The mutation can be a T cell lymphoma mutation or a mutation in a clonally expanded population of T cells. In some embodiments, the mutation is a point mutation or substitution.

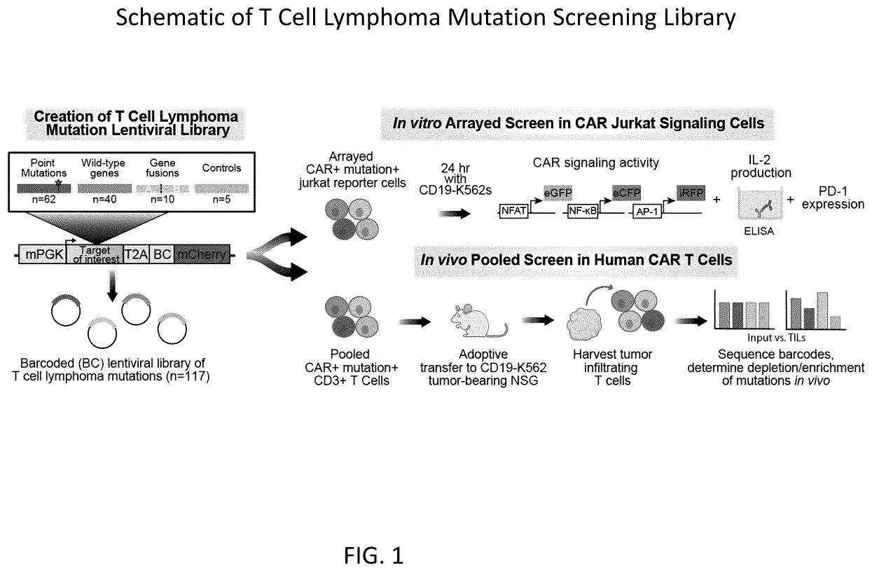

In some embodiments, the mutation is a gene fusion. In some embodiments, the gene fusion comprises a polypeptide comprising a caspase-associated recruitment domain (CARD). In some embodiments, the gene fusion comprises a polypeptide comprising a CARD containing protein or a functional fragment thereof. A functional fragment of a CARD containing protein can be, for example, a fragment that provides NF-κB transcriptional activity, AP-1 transcriptional activity, and/or MALT1 proteolytic activity at a level at least 70%, 75%, 80%, 85%, 90%, or 95% of that of a full-length protein, as determined by the in vitro CAR Jurkat assay shown in FIG. 1 and described herein. In some embodiments, the gene fusion comprises a domain capable of binding to (i) a substrate localized to the intracellular side of the plasma membrane of a cell and/or (ii) a target polypeptide comprising a phosphorylated tyrosine (pTyr). In some embodiments, the gene fusion comprises a CARD containing protein and a domain capable of binding to (i) a substrate localized to the intracellular side of the plasma membrane of a cell and/or (ii) a target polypeptide comprising a pTyr.

In some embodiments, the domain is capable of binding to a substrate indirectly localized to the intracellular side of the plasma membrane. Indirect localization can refer to localization of the substrate to the intracellular side of the plasma membrane through, for example, binding of the substrate to another polypeptide or lipid that is directly localized to the intracellular side of the plasma membrane. Indirect localization can refer to localization of the substrate to the intracellular side of the plasma membrane through, for example, interaction of the substrate with another polypeptide or lipid that is directly localized to the intracellular side of the plasma membrane. In some embodiments, the domain is capable of binding to a substrate directly localized to the intracellular side of the plasma membrane. Direct localization can refer to localization of the substrate to the intracellular side of the plasma membrane through, for example, binding of the substrate to the intracellular side of the plasma membrane itself. Direct localization can refer to localization of the substrate to the intracellular side of the plasma membrane through, for example, interaction of the substrate with the intracellular side of the plasma membrane itself.

In some embodiments, the mutation is a T cell lymphoma mutation. T cell lymphoma mutation can be a mutation occurring in, or identified in, a T cell lymphoma. T cell lymphomas are a heterogeneous group of lymphoid malignancies that occur in nodal and extranodal sites. There are two main types of T cell lymphomas namely T-lymphoblastic lymphoma and Peripheral T-cell lymphomas classified based on clinical manifestations and cytogenetic mutations. Peripheral T cell lymphoma can be further divided into cutaneous T cell lymphoma, adult T cell lymphoma, angioimmunoblastic T cell lymphoma, natural killer T cell lymphoma, enteropathy associated T cell lymphoma, and anaplastic large cell lymphoma. In some embodiments, the T cell lymphoma mutation occurs in a T lymphoblastic lymphoma. In some embodiments, the T cell lymphoma mutation occurs in a Peripheral T cell lymphoma.

In some embodiments, the mutation is a mutation in a clonally expanded population of T cells. As used herein, a clonally expanded population of T cells can be a population of T cells that are descended from a single progenitor cell. T cells within the clonally expanded population can express the same T cell receptor.

The polypeptides of the disclosure, or the recombinant nucleic acid constructs and/or recombinant nucleic acids of the disclosure, can alter T cell signaling in various ways. In some embodiments, the altering of the T cell signaling can be by enhancing, promoting, improving, reducing, regulating, or modulating the signaling pathway within a T cell. In some embodiments, the altering of the T cell signaling can be by activating, increasing, suppressing, inhibiting, or other means of changing the signaling.

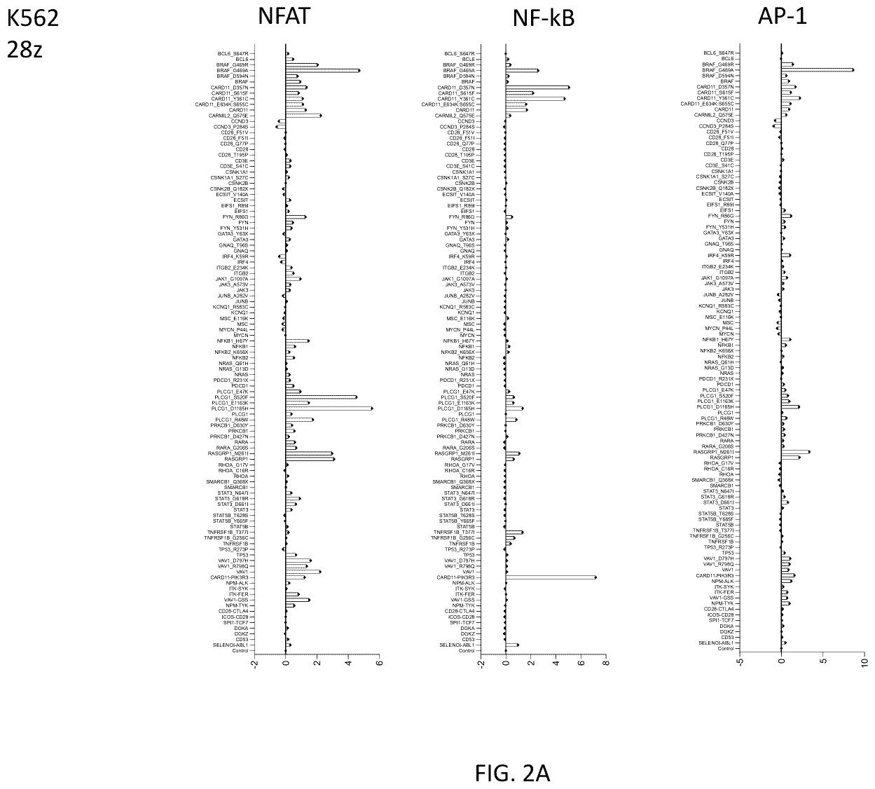

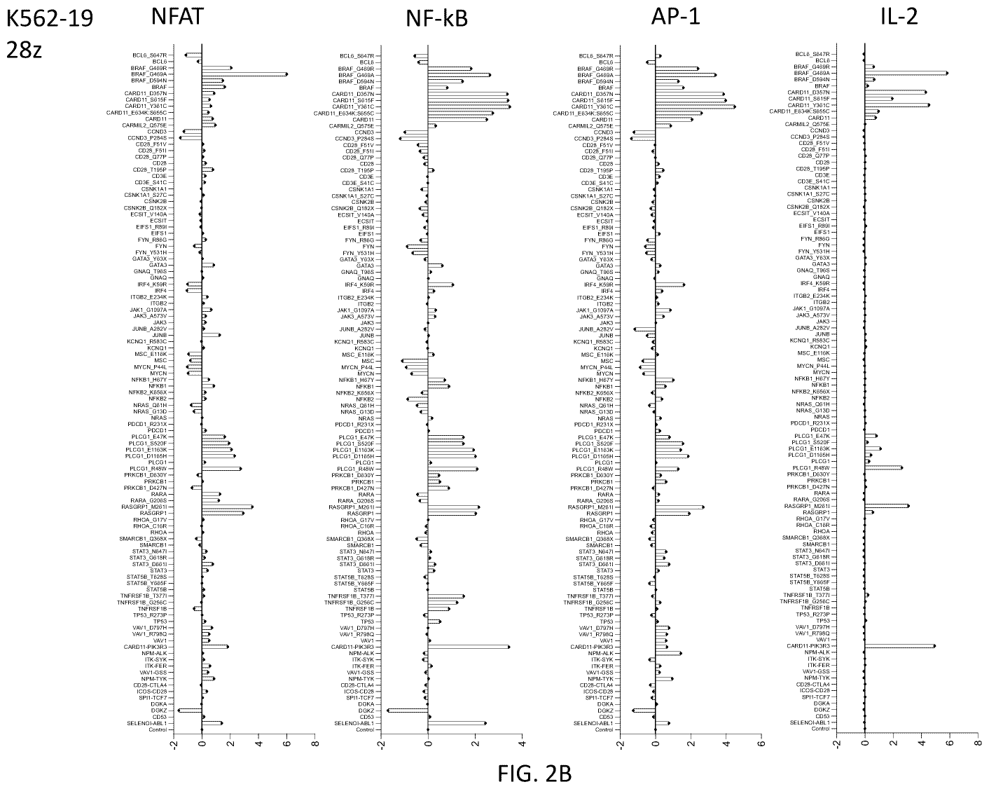

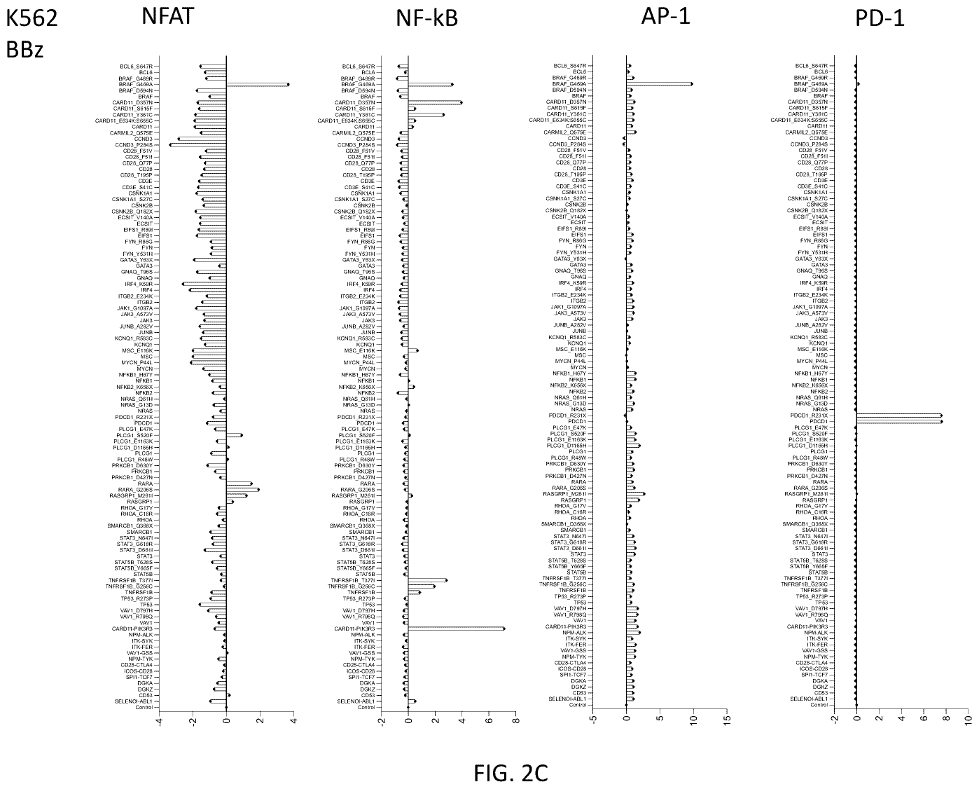

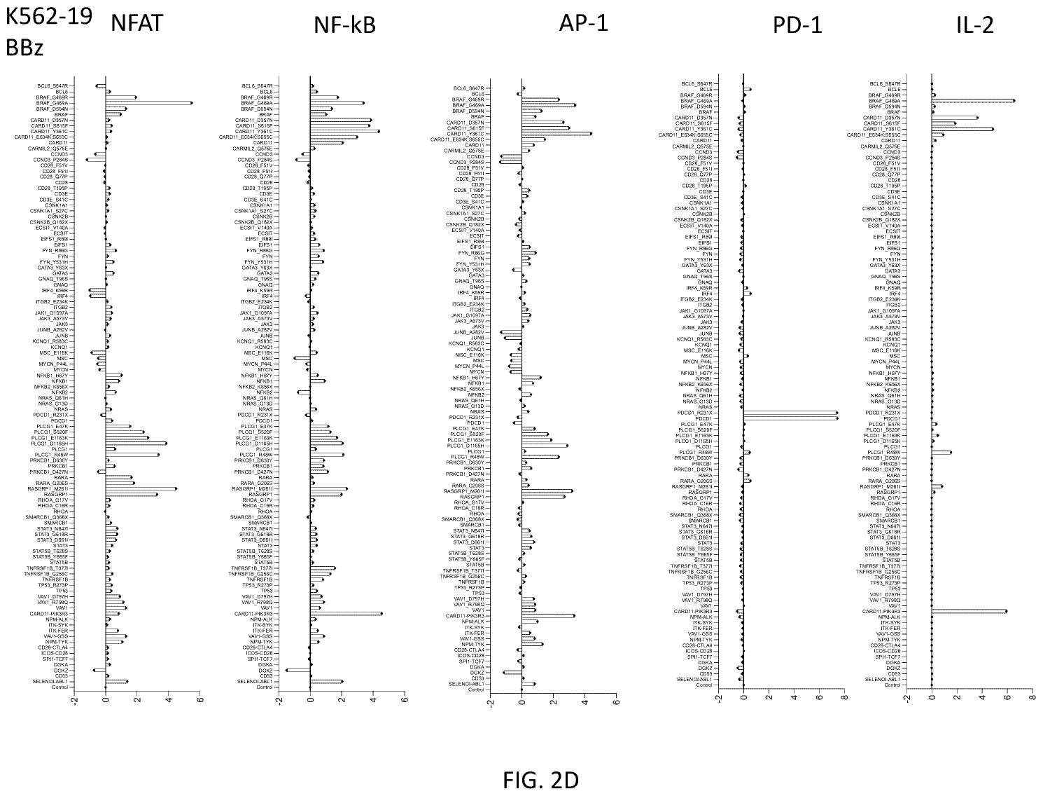

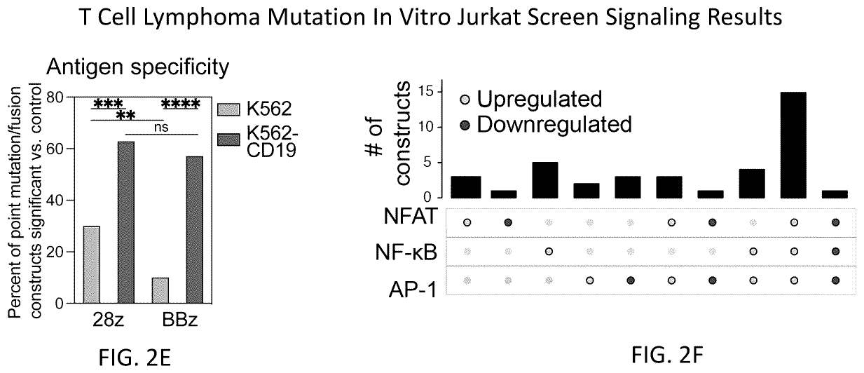

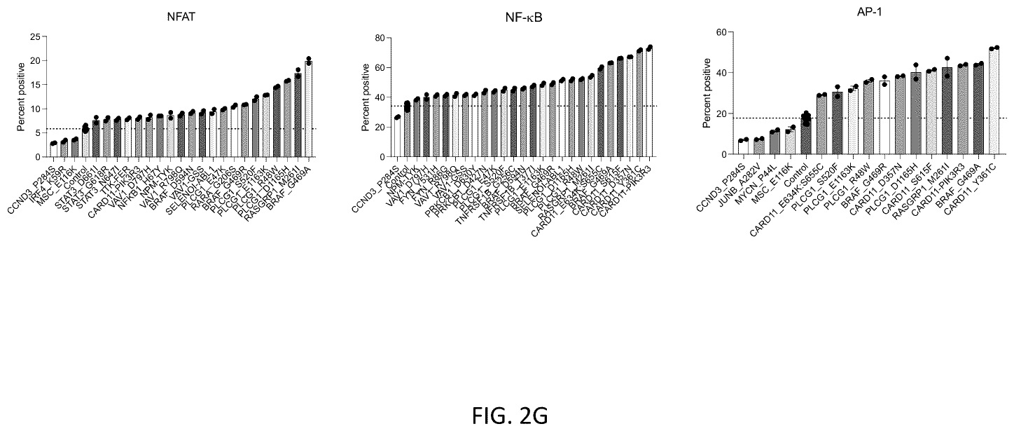

The polypeptides of the disclosure, or the recombinant nucleic acid constructs and/or recombinant nucleic acids of the disclosure, can alter T cell signaling through one or more T cell signaling pathways. In some embodiments, the polypeptides or recombinant nucleic acid constructs and/or recombinant nucleic acids of the disclosure can alter T cell signaling by increasing one or more pathways and/or reducing one or more pathways, or by a combination of enhancing and/or reducing various pathways, for example as shown in FIG. 2F. In some embodiments, the mutation can alter cytokine production. The cytokine can be, without limitation, IL-2, IL-4, IL-5, TNF alpha, IFN-gamma, IL-13 and/or other cytokines. In some embodiments, the cytokine is IL-2. In some embodiments, the cytokine is IL-2, and the production of IL-2 is increased.

In some embodiments, the polypeptides or recombinant nucleic acid constructs and/or recombinant nucleic acids of the disclosure can alter T cell signaling through one or more T cell signaling pathways. Signaling pathways can be, without limitation, the NFAT pathway, NF-κB pathway, AP-1 pathway, JAK/STAT pathway, RAS/MEK/ERK, and/or phospholipase gamma signaling, or described elsewhere herein.

In some embodiments, the polypeptides or recombinant nucleic acid constructs and/or recombinant nucleic acids of the disclosure can alter CARD11-BCL10-MALT1 complex signaling, co-stimulatory molecule signaling, cytokine production and/or transcription factor activity in T cells.

The disclosure also relates to polypeptides having one or more mutations that can reduce T cell exhaustion, increase proliferation, alter effector function, resist T cell dysfunction, increase T cell fitness, enhance in vivo persistence, and/or increase intratumoral presence of therapeutic T cells. The disclosure further relates to recombinant nucleic acids encoding polypeptides having one or more mutations, that can reduce T cell exhaustion, increase proliferation, alter effector function, resist T cell dysfunction, increase T cell fitness, enhance in vivo persistence, and/or increase intratumoral presence of therapeutic T cells.

T cell fitness can refer to the ability of a T cell to generate an immune response. The ability of a T cell to perform T cell functions such as signaling, cytokine production, survival, and persistence in a tumor, may contribute to the fitness of a T cell. T cell exhaustion may contribute to reducing its fitness.

In some embodiments, polypeptides of the disclosure, or the recombinant nucleic acid constructs and/or recombinant nucleic acids of the disclosure can alter in vivo persistence in tumors of therapeutic T cells including the mutation. The in vivo persistence of therapeutic T cells can refer to the length of time that the therapeutic T cells exist within the tumor of the host after infusion. In some embodiments, in vivo persistence of therapeutic T cells is enhanced. Enhanced in vivo persistence of therapeutic T cells can include at least a positive log 2 fold change in therapeutic T cells as compared to the input total.

In some embodiments, the polypeptides or recombinant nucleic acid constructs and/or recombinant nucleic acids of the disclosure can alter the in vivo persistence in tumors or the other functions or activities of T cells described herein, through one or more of the following genes: BCL6, BCOR, BRAF, CARD11, CARMIL2, CCND3, CD28, CD3E, CSNK1A1, CSNK2B, ECSIT, EIFS1, FYN, GATA, GNAQ, IRF4, ITGB2, JAK1, JAK3, JUNB, KCNQ1, LATS1, MSC, MYCN, NFKB1, NFKB2, NRAS, PDCD1, PLCG1, PRKCB1, RARA, RASGRP1, RHOA, SMARCB1, STAT3, STAT5, TBL1XR1, TNFRSF1B, TP53 and VAV1.

In some embodiments, the genes include caspase recruitment domain family member 11 (CARD11). In some embodiments, the genes include capping protein regulator and myosin 1 linker 2 (CARMIL2), mucosa-associated lymphoid tissue lymphoma translocation protein 1 (MALT1), B-cell lymphoma 6 (BCL6), B-cell lymphoma 10 (BCL10), and MYCN. In some embodiments, the gene can be a STAT3, STAT5B, JAK1, JAK2, or JAK3 gene. In some embodiments, the gene can be BRAF, or RASGRP1. In some embodiments, the gene can be phospholipase C gamma 1 (PLCG1) gene. In some embodiments, the gene can be NFKB1, NFKB2, or JUNB gene. In some embodiments, the gene can be TNFRSF1B.

In some embodiments, the polypeptides of the disclosure, or the recombinant nucleic acid constructs and/or recombinant nucleic acids of the disclosure can alter the therapeutic efficacy of engineered T cells. Alteration of therapeutic efficacy can include, without limitation, a decrease in T cell exhaustion, increased proliferative capacity, enhanced anti-tumor effect, enhanced replicative lifespan, decreased replicative senescence, enhanced ability to kill, enhanced fitness of engineered T cells and/or other functions or activities of T cells. In some embodiments, the nucleic acid constructs and/or recombinant nucleic acids of the disclosure encodes a polypeptide comprising an amino acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204, SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ ID NO: 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256 or a variant thereof wherein the amino acid sequence includes one or more of the mutations listed in Table 1 for the same gene.

In some embodiments, the nucleic acids constructs and/or recombinant nucleic acids of the disclosure include a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, or SEQ ID NO: 255 or a variant thereof wherein the variant includes one or more of the mutations listed in Table 1 for the same gene.

In some embodiments, the polypeptides of the disclosure have, or the nucleic acid constructs and/or recombinant nucleic acids of the disclosure encode a polypeptide sequence having, at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 25 and comprising a substitution at an amino acid position 361, 615, 634, 655, 357 or combinations thereof. In some embodiments, the substitution is Y361C, S615F, E634K, S655C and/or D357N. In some embodiments the nucleic acid constructs and/or recombinant nucleic acids of the disclosure encode a polypeptide a sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 25 and comprising at least one, two or more substitutions at an amino acid position 361, 615, 634, 655, 357. In some embodiments, the nucleic acid constructs and/or recombinant nucleic acids encode a polypeptide having E634K and S655C mutations. In some embodiments, the recombinant nucleic acid construct encodes a substitution selected from the group consisting of: Y361C, S615F, E634K, D357N, S655C, and any combination thereof.

In some embodiments, the disclosure relates to polypeptides, or to recombinant nucleic acids encoding polypeptides, comprising a caspase-associated recruitment domain (CARD) containing protein, or a functional fragment thereof. CARD is a conserved homology domain comprising a 6-helix bundle or 5-helix bundle. CARDs can mediate protein-protein interactions between key apoptotic signaling molecules. Non-limiting examples of CARDs include CARD6, CARD8, CARD9, CARD10, CARD11, CARD14, CARD16, CARD18, and CARD19. Non-limiting examples of proteins comprising CARDs include those described in Boyle and Monie bioRxiv 087908; doi: https://doi.org/10.1101/087908; and/or Park. In J Mol Med. 2019 March; 43(3):1119-1127, such as human caspase-1, -2, -4, and -5, mouse caspase-1, -2, -11, and -12, ASC, NOD1, NOD2, Apaf-1, BCL-10, and RIG-I. In some embodiments, the CARD comprises or consists of a sequence having at least 75%, 80%, 85%, 90%, 95%, 96%, 97%, 98%, 99%, or 100% identity to any one of SEQ ID NO: 261-289. In some embodiments, the CARD containing protein, or functional fragment thereof, is derived from a CARD protein selected from CARD9 (UniProt #Q9H257), CARD10 (UniProt #Q9BWT7) CARD11 (UniProt #Q9BXL7), or CARD14 (UniProt #Q9H257) (Wang et al. J Biol Chem. 2001 Jun. 15; 276(24):21405-9; Bertin et al. J Biol Chem. 2001 Apr. 13; 276(15):11877-82.). In some embodiments, the CARD containing protein, or functional fragment thereof, is derived from a CARD11 protein (UniProt #Q9BXL7), or a functional fragment thereof. In some embodiments, the CARD containing protein comprises or consists of a sequence having at least 75%, 80%, 85%, 90%, 95%, 96%, 97%, 98%, 99%, or 100% identity to any one of SEQ ID NOs: 261-264. In some embodiments, the functional fragment of the CARD containing protein is derived from CARD11 and comprises or consists of a sequence having at least 75%, 80%, 85%, 90%, 95%, 96%, 97%, 98%, 99%, or 100% identity to SEQ ID NO: 263.

In some embodiments, the function of the CARD containing protein or the functional fragment thereof is to bind to a CARD domain on BCL10 (Bertin et al. J. Biol Chem. 201 April; 276(15):11877-11882). In some embodiments, the functional fragment thereof comprises at least 40, at least 50, at least 60, at least 70, at least 80, at least 90, at least 100, at least 150, at least 200, at least 300, at least 400, or at least 500 amino acids. Non-limiting examples of a cell include a T cell, a macrophage, a monocyte, and a natural killer (NK) cell. In some embodiments, activation of the cell produces the substrate localized to the intracellular side of the plasma membrane. In some embodiments, the substrate localized to the intracellular side of the plasma membrane of a cell is a phosphoinositide. Non-limiting examples of phosphoinosities include those described in Posor et al. Nat Rev Mol Cell Biol. 2022 December; 23(12):797-816. In some embodiments, the phosphoinositide is selected from phosphatidylinositol (3,4,5)-trisphosphate (PIP3), phosphatidylinositol 4,5-bisphosphate (PI(4,5)P2) (Hawse and Cattley J. Bio. Chem. 2019 March; 294(13):4793-4805; Sun et al. PLoS ONE. 2011 November; 6(11): e27227). In some embodiments, the polypeptide binds to the phosphoinositide with a Kd of less than 100 μM, 50 μM, 10 μM, 5 μM, 1 μM, 0.5 μM, 0.1 μM, 0.05 μM, or 0.01 μM, and wherein the Kd is analyzed using SPR as described in Yu et al. Molecular Cell. 2004 March; 13(5):p677-688.

The disclosure further relates to polypeptides comprising a domain capable of binding to (i) a substrate localized to the intracellular side of the plasma membrane of a cell and/or (ii) a target polypeptide comprising a phosphorylated tyrosine (pTyr). In some embodiments, the domain is capable of binding to a substrate indirectly localized to the intracellular side of the plasma membrane. Indirect localization can refer to localization of the substrate to the intracellular side of the plasma membrane through, for example, binding of the substrate to another polypeptide or lipid that is directly localized to the intracellular side of the plasma membrane. Indirect localization can refer to localization of the substrate to the intracellular side of the plasma membrane through, for example, interaction of the substrate with another polypeptide or lipid that is directly localized to the intracellular side of the plasma membrane. In some embodiments, the domain is capable of binding to a substrate directly localized to the intracellular side of the plasma membrane. Direct localization can refer to localization of the substrate to the intracellular side of the plasma membrane through, for example, binding of the substrate to the intracellular side of the plasma membrane itself. Direct localization can refer to localization of the substrate to the intracellular side of the plasma membrane through, for example, interaction of the substrate with the intracellular side of the plasma membrane itself.

Non-limiting examples of domains capable of binding to (i) and/or (ii) include Src Homology region 2 (SH2) domains, Src Homology region 3 (SH3) domains, pleckstrin homology (PH) domains, and phosphytyrosine-binding (PTB) domains. SH2 and PTB domains mediate protein-protein interactions involved in many signal transduction pathways. SH2, SH3, and PTB domains that can be used in the disclosure include, for example, those disclosed in Schlessinger et al. Sci STKE. 2003 Jul. 15; 2003(191):RE12. SH2 containing proteins can bind to plasma membrane lipids through a different binding pocket than the pTyr binding pocket; most SH2 domains bind plasma membrane lipids and many have high phosphoinositide specificity (Park et al., Cell. 2016 Apr. 7; 62(1):7-20). In some embodiments, the domain is, or comprises, an SH3 domain. In some embodiments, the domain is, or comprises, a PTB domain. In some embodiments, the domain is, or comprises, a PH domain. In some embodiments, the domain is, or comprises, an SH2 domain. In some embodiments, the SH2 domain comprises or consists of a sequence having at least 75%, 80%, 85%, 90%, 95%, 96%, 97%, 98%, 99%, or 100% identity to any one of SEQ ID NOs: 305 and 307-437. In some embodiments, the SH2 domain is from a PIK3R3 protein (UniProt #Q92569). In some embodiments, the SH2 domain comprises the motif of a conserved arginine residue in the FLVR motif (Arg 01B5 or Arg175 in the v-Src SH2 domain). Most conserved resides are clustered on the βB strand, and the conserved arginine residue in the FLVR motif plays the central role in forming a double hydrogen bond with the phosphate group of pTyr. Additional residues that are key for phosphopeptide binding are His PD4, Lys PD6, and Arg αA2, which coordinate and anchor the aromatic ring of the phospho-tyrosine (Diop et al. Int J Mol Sci. 2022 Dec. 15; 23(24):15944). In some embodiments, the SH2 domain comprises or consists of a sequence having at least 75%, 80%, 85%, 90%, 95%, 96%, 97%, 98%, 99%, or 100% identity to SEQ ID NO: 305 In some embodiments, the SH2 domain is an engineered SH2 domain with an enhanced affinity for phosphotyorsine. In some embodiments, the SH2 domain comprises or consists of a sequence having at least 75%, 80%, 85%, 90%, 95%, 96%, 97%, 98%, 99%, or 100% identity to any one of SEQ ID NO: 313-315 (e.g., Veggiani et al. Protein Sci. 2019 February; 28(2):403-413).

Non-limiting examples of target polypeptides comprising a pTyr include P110α/PIK3CA, P110β/PIK3CB, P110δ/PIK3CD, IGF-1R, ErbB2, CTLA-4, and CD28. In some embodiments, the target polypeptide is derived from IGF-1R, CTLA-4, or CD28. In some embodiments, the target polypeptide comprises or consists of a sequence having at least 75%, 80%, 85%, 90%, 95%, 96%, 97%, 98%, 99%, or 100% identity to any one of SEQ ID NOs: 298-304. In some embodiments, the pTyr is located at the position corresponding to pY1346 of SEQ ID NO: 301. In some embodiments, the pTyr is located at the position corresponding to pY1221 of SEQ ID NO: 302. In some embodiments, the polypeptide binds to the target polypeptide with a Kd of less than 100 μM, 50 μM, 10 μM, 5 μM, 1 μM, 0.5 μM, 0.1 μM, 0.05 μM, or 0.01 μM, wherein the Kd is analyzed by fluorescence polarization assay as described in Hause et al. PLoS One. 2012; 7(9):e44471. In some embodiments, the polypeptide has a higher affinity for the target polypeptide comprising the pTyr than a control polypeptide without phosphorylation at the corresponding tyrosine position. In some embodiments, the polypeptide has at least 2-fold, at least 5-fold, at least 10-fold, at least 50-fold, or at least 100-fold, higher affinity (lower Kd) for the target polypeptide comprising the phosphorylated tyrosine than a control polypeptide without phosphorylation at the corresponding tyrosine position.

Furthermore, the disclosure relates to polypeptides comprising a CARD containing protein or a functional fragment thereof, and a domain capable of binding to (i) a substrate localized to the intracellular side of the plasma membrane of a cell and/or (ii) a target polypeptide comprising a phosphorylated tyrosine (also called a CARD fusion polypeptide), as disclosed herein. In some embodiments, the polypeptide comprises a CARD containing protein, or a functional fragment thereof, and an SH2 domain (also called a CARD-SH2 fusion polypeptide). In some embodiments, the disclosure relates to a polypeptide comprising a functional fragment of a CARD containing protein derived from a CARD11 protein and an SH2 domain from a PIK3R3 protein (also called a CARD11-PIK3R3 fusion polypeptide).

Furthermore, the disclosure relates to recombinant nucleic acids encoding polypeptides comprising a CARD containing protein or a functional fragment thereof and a domain capable of binding to (i) a substrate localized to the intracellular side of the plasma membrane of a cell and/or (ii) a target polypeptide comprising a phosphorylated tyrosine, as disclosed herein. In some embodiments, the recombinant nucleic acid encodes a polypeptide comprising a CARD containing protein or a functional fragment thereof and an SH2 domain. In some embodiments, the recombinant nucleic acid encodes a functional fragment of a CARD containing protein derived from a CARD11 protein and an SH2 domain from a PIK3R3 protein. In some embodiments, the recombinant nucleic acid encodes a functional fragment of a CARD containing protein derived from a CARD9 protein and an SH2 domain from a PIK3R3 protein.

In some embodiments, the polypeptide is a CARD11-PIK3R3 fusion polypeptide. In some embodiments, the recombinant nucleic acid constructs and/or recombinant nucleic acids encode a CARD11-PIK3R3 fusion polypeptide or any truncations thereof. In some embodiments, the polypeptide includes, or the nucleic acid constructs and/or recombinant nucleic acid encodes a CARD11-PIK3R3 fusion polypeptide or truncations thereof, including an amino acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOS: 206, SEQ ID NOS: 226, SEQ ID NOS: 228, SEQ ID NOS: 230, SEQ ID NOS: 232, SEQ ID NOS: 234, SEQ ID NOS: 236, SEQ ID NOS: 238, SEQ ID NOS: 240, SEQ ID NOS: 242, SEQ ID NOS: 244, SEQ ID NOS: 248, SEQ ID NOS: 250, SEQ ID NOS: 252, SEQ ID NOS: 254, or SEQ ID NO:256. In some embodiments, the truncations are encoded by a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 205, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 246, SEQ ID NO: 247, SEQ ID NO: 249, SEQ ID NO: 251, SEQ ID NO: 253, or SEQ ID NO: 255.

The disclosure further relates to a second polypeptide portion derived from PIK3R3. Non-limiting examples of polypeptides derived from PIK3R3 include either of two SH2 domains, a catalytic subunit, and a regulatory subunit. In some embodiments, the second polypeptide portion comprises or consists of a sequence having at least 75%, 80%, 85%, 90%, 95%, 96%, 97%, 98%, 99%, or 100% identity to any one of SEQ ID NO: 205, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 246, SEQ ID NO: 247, SEQ ID NO: 249, SEQ ID NO: 251, SEQ ID NO: 253, and SEQ ID NO: 255. In some embodiments, the polypeptide does not comprise a Coiled-coil domain or a portion thereof. In some embodiments, the polypeptide comprises a Coiled-coil domain or a portion thereof. In some embodiments, the Coiled-coil domain comprises or consists of a sequence having at least 75%, 80%, 85%, 90%, 95%, 96%, 97%, 98%, 99%, or 100% identity to any one of SEQ ID NOs: 290-294. In some embodiments, the Coiled-coil domain comprises or consists of a sequence having at least 75%, 80%, 85%, 90%, 95%, 96%, 97%, 98%, 99%, or 100% identity to SEQ ID NO: 290. In some embodiments, the polypeptide comprises or consists of about 10, about 20, about 30, about 40, about 50, about 60, about 80, about 100, about 120, about 140, about 150, about 160, about 180, about 200, about 220, about 240, about 250, about 260, about 280, or about 300 amino acids of the N-terminal portion of the Coiled-coil domain. In some embodiments, the polypeptide comprises no more than 10, 20, 30, 40, 50, 60, 80, 100, 120, 140, 150, 160, 180, 200, 220, 240, 250, 260, 280, or 300 amino acids of the N-terminal portion of the Coiled-coil domain.

The present disclosure further relates to polypeptides wherein the domain capable of binding to the target polypeptide comprising the phosphorylated tyrosine, or the SH2 domain, or the second polypeptide portion, is located at the N-terminus of the CARD containing protein or functional fragment thereof, between the CARD containing protein and the Coiled-coil domain, or at the C-terminus of the CARD containing protein and/or the Coiled-coil domain. In some embodiments, the polypeptide comprises the CARD domain derived from a CARD11 protein followed by the Coiled-coil domain derived from the CARD11 protein. The present disclosure further relates to polypeptides wherein the domain capable of binding to the target polypeptide comprising the phosphorylated tyrosine, or the SH2 domain, or the second polypeptide portion, is located close to the C-terminus of the polypeptide, wherein the polypeptide has no more than 50, 40, 30, 20, 15, 10, or 5 amino acids at the C-terminus of the domain in (b), or the SH2 domain, or the second polypeptide portion.

In some embodiments, the polypeptide does not comprise an inhibitory domain (ID) or a portion thereof. In other embodiments, the polypeptide comprises an ID or a portion thereof. In some embodiments, the ID comprises or consists of a sequence having at least 75%, 80%, 85%, 90%, 95%, 96%, 97%, 98%, 99%, or 100% identity to SEQ ID NO: 294. In some embodiments, the inhibitory domain (ID) comprises or consists of a sequence having at least 75%, 80%, 85%, 90%, 95%, 96%, 97%, 98%, 99%, or 100% identity to SEQ ID NO: 295. In some embodiments, the polypeptide comprises or consists of about 10, about 20, about 30, about 40, about 50, about 60, about 80, about 100, about 120, about 140, about 150, about 160, about 180, or about 200 amino acids of the N-terminal portion of the ID. In some embodiments, the second polypeptide portion comprises no more than 10, 20, 30, 40, 50, 60, 80, 100, 120, 140, 150, 160, 180, or 200 amino acids of the N-terminal portion of the ID. In some embodiments, the polypeptide does not comprise a sequence that is at least 90%, 95%, 96%, 97%, 98%, 99%, or 100% identical to SEQ ID NO: 297. In some embodiments, the polypeptide comprises one or more mutations corresponding to S615F, D357N, Y361C, E634K, and/or S655C of SEQ ID NO: 26.

In some embodiments, the CARD11-PIK3R3 fusion polypeptide includes a CARD domain (i.e., containing protein or functional fragment thereof), a CARD inhibitory domain (ID), a coiled-coil domain, and an SH2 domain from PIK3R3. In some embodiments, the CARD11-PIK3R3 fusion polypeptide includes a functional fragment of a CARD containing protein derived from CARD11 domain, a coiled-coil domain, and an SH2 domain from PIK3R3.

In some embodiments, the recombinant nucleic acid constructs and/or recombinant nucleic acids of the disclosure, includes a CARD11-PIK3R3 fusion polypeptide encoded by a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 205, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 246, SEQ ID NO: 247, SEQ ID NO: 249, SEQ ID NO: 251, SEQ ID NO: 253, SEQ ID NO: 255, or SEQ ID NO: 296.

In some embodiments, the polypeptide of the disclosure has, or the recombinant nucleic acid constructs and/or recombinant nucleic acids of the disclosure encodes, a polypeptide comprising a BCL3 polypeptide having a substitution at amino acid 647. In some embodiments of the recombinant nucleic acid constructs and/or recombinant nucleic acids of the disclosure, the polypeptide includes an amino acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 4 and including a substitution at amino acid 647. In some embodiments, the substitution at amino acid 647 of SEQ ID NO: 4 is S647R.

In some embodiments, the polypeptide of the disclosure has, or the recombinant nucleic acid constructs and/or recombinant nucleic acids of the disclosure encodes, a polypeptide comprising a CARMIL2 polypeptide having a substitution at amino acid 575. In some embodiments, the polypeptide includes an amino acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 28 and including a substitution at amino acid 575. In some embodiments, the substitution at amino acid 575 of SEQ ID NO: 28 is Q575E.

In some embodiments, the polypeptide of the disclosure has, or the recombinant nucleic acid constructs and/or recombinant nucleic acids of the disclosure encodes a polypeptide comprising a MYCN polypeptide having a substitution at amino acid 44. In some embodiments, the polypeptide includes an amino acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 114 and including a substitution at amino acid 44. In some embodiments, the substitution at amino acid 44 of SEQ ID NO: 114 is P44L.

In some embodiments, the polypeptide is encoded by a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:1. SEQ ID NO: 111 or SEQ ID 129. In some embodiments, the polypeptide includes a mutation capable of (i) altering JAK/STAT signaling in T cells (ii) altering cytokine production, and/or (iii) enhancing in vivo persistence of therapeutic T cells comprising the mutation in tumors. In some embodiments the mutation is in a JAK1, JAK3 STAT3, or STAT5 gene. In some embodiments, the polypeptide includes an amino acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to (i) SEQ ID NO: 90 and comprising a substitution at amino acid position 1097 of SEQ ID NO: 90, (ii) SEQ ID NO: 94 and comprising a substitution at an amino acid position 573 of SEQ ID NO: 94, (iii) SEQ ID NO: 176 and comprising a substitution at an amino acid position 618, 647, or 661 of SEQ ID NO: 176 or a combination thereof, (iv) SEQ ID NO: 182 and comprising a substitution at an amino acid position 628, or at amino acid position 665 of SEQ ID NO: 182 or a combination thereof.

In some embodiments of the recombinant nucleic acid constructs and/or recombinant nucleic acids of the disclosure, the nucleic acid construct includes a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:87 wherein the nucleic acid sequence encodes a JAK1 polypeptide with a G1097A substitution.

In some embodiments of the recombinant nucleic acid constructs and/or recombinant nucleic acids of the disclosure, the nucleic acid construct includes a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 91 wherein the nucleic acid encodes a JAK3 polypeptide with an A573V substitution.

In some embodiments of the recombinant nucleic acid constructs and/or recombinant nucleic acids of the disclosure, the nucleic acid construct includes a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:169 and encoding a STAT3 polypeptide having an N647I substitution.

In some embodiments of the recombinant nucleic acid constructs and/or recombinant nucleic acids of the disclosure, the nucleic acid construct includes a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 171 and encoding a STAT3 polypeptide having a G618R substitution.

In some embodiments of the recombinant nucleic acid constructs and/or recombinant nucleic acids of the disclosure, the nucleic acid construct includes a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 173 and encoding a STAT3 polypeptide having a D661I substitution.

In some embodiments of the recombinant nucleic acid constructs and/or recombinant nucleic acids of the disclosure, the nucleic acid construct includes a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 177 and encoding a STAT5 polypeptide with a T628S substitution.

In some embodiments of the recombinant nucleic acid constructs and/or recombinant nucleic acids of the disclosure, the nucleic acid construct includes a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 179 and encoding a STAT5 polypeptide with a Y665F substitution.

In some embodiments of the recombinant nucleic acid constructs and/or recombinant nucleic acids of the disclosure, the polypeptide includes a mutation capable of altering co-stimulatory molecule signaling in T cells and persistence in tumors of T cells including the mutation. In some embodiments, the mutation is in a TNFR2, TNFRS1B, CD28, ICOS, CTLA4 gene, or a combination thereof.

In some embodiments of the recombinant nucleic acid constructs and/or recombinant nucleic acids of the disclosure, the nucleic acid encodes a polypeptide comprising a TNFRSF1B polypeptide having a substitution at amino acid 256 and/or 377. In some embodiments of the recombinant nucleic acid constructs and/or recombinant nucleic acids of the disclosure, the polypeptide includes an amino acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 192 and comprising a substitution at an amino acid position 256 and/or at position 377 of SEQ ID NO: 192 or a combination thereof.

In some embodiments of the recombinant nucleic acid constructs and/or recombinant nucleic acids of the disclosure, the nucleic acid encodes a polypeptide comprising a TNFRSF1B polypeptide having a substitution at amino acid 337. In some embodiments of the recombinant nucleic acid constructs and/or recombinant nucleic acids of the disclosure, the nucleic acid construct includes a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 187 and encoding a TNFRSF1B polypeptide with a T337I substitution.

In some embodiments of the recombinant nucleic acid constructs and/or recombinant nucleic acids of the disclosure, the nucleic acid encodes a polypeptide comprising a TNFRSF1B polypeptide having a substitution at amino acid 256. In some embodiments of the recombinant nucleic acid constructs and/or recombinant nucleic acids of the disclosure, the nucleic acid construct includes a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 189 and encoding a TNFRSF1B polypeptide with a G256C substitution.

In some embodiments of the recombinant nucleic acid constructs and/or recombinant nucleic acids of the disclosure, the nucleic acid encodes a polypeptide comprising a CD28 polypeptide having a substitution at amino acid 51 and/or 77. In some embodiments of the recombinant nucleic acid constructs and/or recombinant nucleic acids of the disclosure, the polypeptide includes an amino acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 42 and encoding a CD28 polypeptide including a substitution at an amino acid position 51 and/or 77 of SEQ ID NO: 42 or a combination thereof.

In some embodiments of the recombinant nucleic acid constructs and/or recombinant nucleic acids of the disclosure, the nucleic acid construct includes a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 35 and encoding a CD28 polypeptide with a F51V substitution.

In some embodiments of the recombinant nucleic acid constructs and/or recombinant nucleic acids of the disclosure, the nucleic acid construct includes a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 37 and encoding a CD28 polypeptide with a F511 substitution.

In some embodiments of the recombinant nucleic acid constructs and/or recombinant nucleic acids of the disclosure, the nucleic acid construct includes a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 39 and encoding a CD28 polypeptide with a Q77P substitution.

In some embodiments of the recombinant nucleic acid constructs and/or recombinant nucleic acids of the disclosure, the nucleic acid construct includes a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:43 and encoding a CD28 polypeptide with a T195P substitution.

In some embodiments of the recombinant nucleic acid constructs and/or recombinant nucleic acids of the disclosure, the nucleic acid encodes a polypeptide comprising an ICOS-CD28 polypeptide. In some embodiments of the recombinant nucleic acid constructs and/or recombinant nucleic acids of the disclosure, the polypeptide includes an amino acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 220.

In some embodiments of the recombinant nucleic acid construct of the disclosure, the nucleic acid construct includes a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:219.

In some embodiments of the recombinant nucleic acid constructs and/or recombinant nucleic acids of the disclosure, the nucleic acid encodes a polypeptide comprising a CD28-CTLA4 polypeptide. In some embodiments of the recombinant nucleic acid constructs and/or recombinant nucleic acids of the disclosure, the polypeptide includes an amino acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 218.

In some embodiments of the recombinant nucleic acid constructs and/or recombinant nucleic acids of the disclosure, the nucleic acid construct includes a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:217

In some embodiments of the recombinant nucleic acid constructs and/or recombinant nucleic acids of the disclosure, the polypeptide includes a mutation capable of altering RAS/MEK/ERK signaling in T cells and in vivo persistence in tumors of therapeutic T cells including the mutation. In some embodiments, the mutation is in a BRAF gene. In some embodiments, the polypeptide includes an amino acid sequence having at least about 75%, 80%, 85%, 90%, 95%, 96%, 97%, 98%, 99% or 100% sequence identity to SEQ ID NO: 16, and including an amino acid substitution at amino acid position 469 or 594 or a combination thereof. In some embodiments, the amino acid substitution is G469R. In some embodiments, the amino acid substitution is G469A. In some embodiments, the substitution is D594N.

In some embodiments of the recombinant nucleic acid constructs and/or recombinant nucleic acids of the disclosure, the nucleic acid construct includes a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 9 and encoding a BRAF polypeptide with a G469R substitution. In some embodiments of the recombinant nucleic acid constructs and/or recombinant nucleic acids of the disclosure, the nucleic acid construct includes a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:11 and encodes a BRAF polypeptide with a G469A substitution.

In some embodiments of the recombinant nucleic acid constructs and/or recombinant nucleic acids of the disclosure, the nucleic acid construct includes a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:13 and encodes a BRAF polypeptide with a D594N substitution.

In some embodiments n some embodiments of the recombinant nucleic acid constructs and/or recombinant nucleic acids of the disclosure, the polypeptide includes a mutation capable of altering RAS/MEK/ERK signaling in T cells and in vivo persistence in tumors of therapeutic T cells including the mutation. In some embodiments, the mutation is in a RASGRP1 gene. In some embodiments, the polypeptide includes an amino acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 158 and including a substitution at amino acid position 261.

In some embodiments of the recombinant nucleic acid constructs and/or recombinant nucleic acids of the disclosure, the nucleic acid construct includes a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 157 and encoding a RASGRP1 polypeptide with a M261I substitution.

In some embodiments of the recombinant nucleic acid constructs and/or recombinant nucleic acids of the disclosure, the polypeptide includes a mutation capable of altering phospholipase gamma signaling and/or (ii) cytokine production, and/or (iii) in vivo persistence in tumors of therapeutic T cells comprising the mutation. In some embodiments, the mutation is in a Phospholipase C gamma 1 (PLCG1) gene. In some embodiments, the polypeptide includes an amino acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 142 and including a substitution at amino acid position 47, 48, 520, 1163, 1165 or any combination thereof. In some embodiments, the substitution is E47K. In some embodiments, the substitution is R48W. In some embodiments, the substitution is S520F. In some embodiments, the substitution is E1163K. In some embodiments, the substitution is D1165H.

In some embodiments, the recombinant nucleic acid construct includes a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 133 and encodes a PLCG1 polypeptide having a E47K substitution.

In some embodiments, the recombinant nucleic acid construct includes a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 135 and encodes a PLCG1 polypeptide with a S520F substitution.

In some embodiments, the recombinant nucleic acid construct includes a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to, SEQ ID NO: 137 and encodes a PLCG1 polypeptide with a substitution E1163K.

In some embodiments, the recombinant nucleic acid construct includes a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:139 and encodes a PLCG1 polypeptide with a D1165H substitution.

In some embodiments, the recombinant nucleic acid construct includes a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 143 and encodes a PLCG1 polypeptide with a R48W substitution.

In some embodiments of the recombinant nucleic acid constructs and/or recombinant nucleic acids of the disclosure, the polypeptide includes a mutation capable of altering a transcription factor activity in T cells including the mutation. In some embodiments, the mutation is in a NFKB1, NFKB2 or JUNB gene.

In some embodiments of the recombinant nucleic acid constructs and/or recombinant nucleic acids of the disclosure, the nucleic acid encodes a polypeptide comprising an NFKB1 polypeptide having a substitution at amino acid 67. In some embodiments, the polypeptide includes an amino acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 118 and including a substitution at amino acid 67 of SEQ ID NO:118. In some embodiments, the substitution is H67Y.

In some embodiments, the recombinant nucleic acid construct includes a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:115 and encodes a NFKB1 polypeptide with a H67Y substitution.

In some embodiments of the recombinant nucleic acid constructs and/or recombinant nucleic acids of the disclosure, the nucleic acid encodes a polypeptide comprising an NFKB2 polypeptide having a substitution at amino acid 656. In some embodiments, the polypeptide includes an amino acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 122 and including a substitution at amino acid 656 of SEQ ID NO:122. In some embodiments, the substitution is K656X.

In some embodiments, the recombinant nucleic acid construct includes a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 119 and encodes a NFKB2 polypeptide with a K656X mutation.

In some embodiments of the recombinant nucleic acid constructs and/or recombinant nucleic acids of the disclosure, the nucleic acid encodes a polypeptide comprising a JUNB polypeptide having a substitution at amino acid 282. In some embodiments, the polypeptide includes an amino acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 98 and including a substitution at amino acid 282 of SEQ ID NO:98. In some embodiments, the substitution is A282V.

In some embodiments, the recombinant nucleic acid construct includes a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 95 and encodes a JUNB polypeptide with a A282V substitution.

In some embodiments, the polypeptide includes a first polypeptide encoding a partial CARD 11 polypeptide and a second polypeptide encoding a partial PIK3R3 polypeptide. In some embodiments, the first polypeptide includes an amino acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 258. In some embodiments, the first polypeptide includes an amino acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 260.

In some embodiments, the recombinant nucleic acid constructs and/or recombinant nucleic acids of the disclosure includes a promoter. The promoter can be any promoter. The promoter can be a T cell promoter, or an inducible promoter. The promoter can be active in a subset of T cells.

In some embodiments, the promoter can be a constitutive promoter. Examples of constitutive promoters include but are not limited to a MND promoter, EF1a promoter, sEF1 promoter, gamma retroviral LTR promoter, CD4 promoter, CD8a promoter, CD8b promoter, TCRa promoter, TCRb promoter, CD3d promoter, CD3g promoter, CD3e promoter, or a CD3z promoter. In some embodiments, the promoter is a minimal TATA promoter, a pGK, actin promoter, CD25 promoter, IL2 promoter, IL7 promoter, IL15 promoter, KLRG-1 promoter, HLA-DR promoter, CD38 promoter, CD69 promoter, Ki-67 promoter, CD11a promoter, CD58 promoter, CD99 promoter, CD62L promoter, CD103 promoter, CCR4 promoter, CCR5 promoter, CCR6 promoter, CCR9 promoter, CCR10 promoter, CXCR3 promoter, CXCR4 promoter, CLA promoter, Granzyme A promoter, Granzyme B promoter, Perforin promoter, CD57 promoter, CD161 promoter, IL-18Ra promoter, CD69 promoter, GzmB promoter, T-bet promoter, IFNgamma promoter, TIM3 promoter, IL4 promoter, GATA3 promoter, IL1 promoter, IL5 promoter, IL6 promoter, IL13 promoter, IL10 promoter, IL17A promoter, IL6 promoter, IL21 promoter, IL23R promoter, FoxP3 promoter, CTLA4 promoter, CD25 promoter, PD1 promoter, CD45RO promoter, CCR7 promoter, CD28 promoter, CD95 promoter, CD28 promoter, CD27 promoter, CD127 promoter, PD-1 promoter, CD122 promoter, CD132 promoter, c-Kit promoter, nuclear factor of activated T cells (NFAT) promoter, programmed death 1 (PD-1) promoter, T cell immunoglobulin mucin-3 (TIM-3) promoter, cytotoxic T lymphocyte antigen-4 (CTLA4) promoter, lymphocyte-activation protein 3 (LAG-3) promoter, tumor necrosis factor (TNF)-related apoptosis-inducing ligand (TRAIL) promoter, B- and T-lymphocyte attenuator (BTLA) promoter, CD25 promoter, CD69 promoter, Fas ligand (FasL) promoter, TIGIT promoter, TGF-beta promoter, T-bet promoter, Eomes promoter, GATA3 promoter, CD45RA promoter, 2B4 promoter, Type I interferon (IFN) alpha, Type I IFN beta promoter, IFN gamma promoter, IRF3 promoter, IRF7 promoter, NF-κB promoter, AP-1 promoter, TNF-alpha promoter, and CD130 promoter, NR4A1 promoter, NR4A2, or NR4A3 promoter.

The disclosure also provides vectors including the recombinant nucleic acid constructs and/or recombinant nucleic acids of the disclosure.

The nucleic acid molecules can be contained within a vector that is capable of directing their expression in, for example, a cell that has been transformed/transduced with the vector. Suitable vectors for use in eukaryotic and prokaryotic cells are known in the art and are commercially available, or readily prepared by a skilled artisan. See for example, Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference).

DNA vectors can be introduced into eukaryotic cells via conventional transformation or transfection techniques. Suitable methods for transforming or transfecting host cells can be found in Sambrook et al. (2012, supra) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection.

In some embodiments, the expression vector can be a viral vector.

### Host Cells

The nucleic acid of the present disclosure can be introduced into a host cell, such as, for example, a human T lymphocyte, to produce a recombinant or engineered cell containing the nucleic acid molecule. Accordingly, some embodiments of the disclosure relate to methods for making a recombinant or engineered cell, including (a) providing a cell capable of protein expression and (b) contacting the provided cell with a recombinant nucleic acid of the disclosure.

Introduction of the nucleic acid molecules of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, the nucleic acid molecules can be delivered by viral or non-viral delivery vehicles known in the art. For example, the nucleic acid molecule can be stably integrated in the host genome, or can be episomally replicating, or present in the recombinant host cell as a mini-circle expression vector for transient expression. Accordingly, in some embodiments, the nucleic acid molecule is maintained and replicated in the recombinant host cell as an episomal unit. In some embodiments, the nucleic acid molecule is stably integrated into the genome of the recombinant cell. Stable integration can be achieved using classical random genomic recombination techniques or with more precise techniques such as guide RNA-directed CRISPR/Cas9 genome editing, or DNA-guided endonuclease genome editing with NgAgo (Natronobacterium gregoryi Argonaute), or TALENs genome editing (transcription activator-like effector nucleases). In some embodiments, the nucleic acid molecule is present in the recombinant host cell as a mini-circle expression vector for transient expression.

The nucleic acid molecules can be encapsulated in a viral capsid or a lipid nanoparticle, or can be delivered by viral or non-viral delivery means and methods known in the art, such as electroporation. For example, introduction of nucleic acids into cells may be achieved by viral transduction. In a non-limiting example, adeno-associated virus (AAV) is engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral-derived vector systems are also useful for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile; and (vii) a relatively easy system for vector manipulation and production.

In some embodiments, host cells can be genetically engineered (e.g., transduced or transformed or transfected) with, for example, a vector construct of the present application that can be, for example, a viral vector or a vector for homologous recombination that includes nucleic acid sequences homologous to a portion of the genome of the host cell, or can be an expression vector for the expression of the polypeptides of interest. Host cells can be either untransformed cells or cells that have already been transfected with at least one nucleic acid molecule.

Certain aspects of the disclosure relate to cells comprising the constructs and or/recombinant nucleic acid or the vectors of the disclosure. In some embodiments, the recombinant cell is a prokaryotic cell or a eukaryotic cell. In some embodiments, the cell is in vivo. In some embodiments, the cell is ex vivo. In some embodiments, the cell is in vitro. In some embodiments, the recombinant cell is a eukaryotic cell. In some embodiments, the recombinant cell is an animal cell. In some embodiments, the animal cell is a mammalian cell. In some embodiments, the animal cell is a human cell. In some embodiments, the cell is a non-human primate cell. In some embodiments, the cell is a non-natural cell or has been genetically engineered. In some embodiments, the cell is not a cancerous cell. In some embodiments, the recombinant nucleic acid is exogenous. In some embodiments, the mammalian cell is an immune cell, a neuron, an epithelial cell, and endothelial cell, or a stem cell. In some embodiments, the cell is not a CD4+ T cell. In some embodiments, the cell can be an immune cell, a T cell, a CD4+ cell, a CD8+ cell, a regulatory T cell, a gamma delta T cell, an invariant iNKT cell, a MAIT cell, a macrophage, a monocyte, a natural killer cell (NK), or a tumor infiltrating lymphocyte (TIL). In some embodiments, the cell comprises at least one copy, or at least two copies, of endogenous nucleic acid sequence encoding a CARD11 protein, or a protein comprising a CARD11 CARD domain without any SH2 domain. In some embodiments, the recombinant nucleic acid of the cell is located at the endogenous CARD11-encoding gene locus, or comprises at least a portion of the endogenous CARD11-encoding gene, of the cell.

The T cells of the disclosure or precursors thereof can be immune cells of the lymphoid lineage. In some embodiments, the T cell expresses an engineered immune receptor that binds to a target on a tumor cell. In some embodiments, the T cell can express a T cell receptor (TCR). A TCR as known by the skilled in the art can be composed of two different, an alpha chain and a beta chain, each consisting of a constant region that anchors the chain inside the T cell surface membrane, and a variable region which can recognize and bind an antigen presented by MHCs. The TCR complex can be associated with six polypeptides forming two heterodimers, CD3γE and CD3δE, and one homodimer, CD3ζ, which together forms the CD3 complex. TCRs can be engineered to utilize the modification of T cells that retain these complexes to specifically target the antigens expressed by particular tumor cells. As used herein, a TCR can be a naturally-occurring or an engineered TCR.

In some embodiments, the T cells can be CD4+ or CD8+ and can include, but are not limited to, regulatory T cells, cytotoxic T lymphocytes, T helper cells, and memory T cells, including central memory T cells (TCM), stem memory T cells (TSCM), stem-cell-like memory T cells (or stem-like memory T cells), and effector memory T cells such as, for example, TEM cells and TEMRA (CD45RA+) cells, effector T cells, Th1 cells, Th2 cells, Th9 cells, Th17 cells, Th22 cells, Tfh (follicular helper) cells, T regulatory cells, natural killer T cells, mucosal associated invariant T cells (MAIT), and γδ T cells. Major T cell subtypes include TSCM (stem cell memory), TCM (central memory), TTM (Transitional Memory), TEM (Effector memory), TTE (Terminal Effector) and TN (naive).

In some embodiments, the T cells of the disclosure, or precursors thereof, can be cells that mediate an immune response, i.e. immunostimulatory cells. Immunostimulatory T cells include, but are not limited to, T helper cells (CD4+), cytotoxic T cells and memory T cells, including central memory T cells (TCM), stem memory T cells (TSCM), stem-cell-like memory T cells (or stem-like memory T cells), and effector memory T cells, for example, TEM cells and TEMRA (CD45RA+) cells, effector T cells, Th1 cells, Th2 cells, Th9 cells, Th17 cells, Th22 cells, Tfh (follicular helper) cells, natural killer T cells, mucosal associated invariant T cells (MAIT), and T6 T cells.

In some embodiments, the T cells of the disclosure can be immunoinhibitory cells, i.e., cells that inhibit an immune response. Immunoinhibitory T cells include regulatory T cells (T regulatory cells, Treg) and follicular regulatory T cells (Tfh) cells.

In some embodiments, the T cells of the disclosure can be hematopoietic stem and/or progenitor cells of the lymphoid lineage that can differentiate into T cells. Hematopoietic stem and/or progenitor cells can be derived from bone marrow, umbilical cord blood, and adult peripheral blood.

In some aspects, the cell further includes (i) a chimeric antigen receptor (CAR) having specificity for a target antigen; and/or (ii) a T cell receptor (TCR) having specificity for a target antigen.

In some embodiments, the T cells of the disclosure can be engineered to express a transgene, such as a CAR or a transcriptional regulator. Transcriptional regulators include synthetic receptors such as the synthetic notch receptors described in U.S. Pat. No. 11,202,801 or other synthetic receptors, such as, for example, those described in provisional application nos: 62/905,251, 63/15,428, 62/905,268, 62/905,263, 62/935,024 and 62/905,248, which are herein incorporated by reference in their entirety.

The T cells can be genetically engineered for recombinant expression of a transgene. Such T cells can but need not express a CAR or a transcriptional receptor that binds to a target antigen, since the cells already are target antigen-specific so that their immune response (for example, cytotoxicity) is stimulated specifically by such target antigen. Such T cells that recognize and are sensitized to a target antigen can be obtained by known methods, by way of example, in vitro sensitization methods using naive T cells or hematopoietic progenitor cells (described, for example, by Wolfl et al., Nat. Protocols 9:950-966 (2014), or by van Lent et al., J Immunol. 179:4959-4968 (2007)), or obtained from a subject that has been exposed to and is mounting an immune response against the target antigen (i.e., in vivo sensitized T cells).

In some aspects, the cell, e.g., T cell, NK cell, and/or TIL, comprises a CAR. In some aspects, the cell that can be prepared to express a CAR (e.g., a CAR T cell) is, e.g., a CD8+ T cell or CD4+ T cell. In some aspects, a CAR-expressing cell disclosed herein is a CAR T cell, e.g., a mono CAR T cell, a genome-edited CAR T cell, a dual CAR T cell, or a tandem CAR T cell.

In some embodiments, the target antigen can be cell surface receptors, adhesion proteins, integrins, mucins, lectins, tumor-associated antigens, and tumor-specific antigens.

In some embodiments, the target antigen can be a tumor-associated antigen. Non-limiting exemplary tumor-associated antigens suitable for the compositions and methods of the disclosure include CD19, B7H3 (CD276), BCMA (CD269), ALPPL2, Claudin 18.2, CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, DLL-3, EGFR, EGFRvIII, ELF2M, EpCAM, EphA2, Ephrin B2, FAP, FLT3, GCC, GD2, GD3, GM3, GPRC5D, HER2 (ERBB2/neu), IGLL1, IL-11Ra, KIT (CD117), KLK2, LY6G6D, MUC1, NCAM, p53R175H, PAP, PDGFR-0, PRAME, PRSS21, PSCA, PSMA, ROR1, SIRPα, SSEA-4, TAG72, TEM1/CD248, TEM7R, TSHR, VEGFR2, ALPI, citrullinated vimentin, cMet, and Axl.

In some embodiments, the target antigen can be CD1, CD1a, CD1b, CD1c, CD1d, CD1e, CD2, CD3d, CD3e, CD3g, CD3F, CD4, CD5, CD7, CD8a, CD8b, CD19, CD20, CD21, CD22, CD23, CD24, CD25, CD27, CD28, CD30, CD33, CD34, CD38, CD40, CD44v6, CD45, CD48, CD52, CD59, CD66, CD70, CD71, CD72, CD73, CD79A, CD79B, CD80 (B7.1), CD86 (B7.2), CD94, CD95, CD97, CD123, CD134, CD140 (PDGFR4), CD152, CD154, CD158, CD171, CD178, CD179, CD179a, CD181 (CXCR1), CD182 (CXCR2), CD183 (CXCR3), CD210, CD246, CD252, CD253, CD261, CD262, CD273 (PD-L2), CD274 (PD-L1), CD276 (B7H3), CD279, CD295, CD339 (JAG1), CD340 (HER2), CEA, CLL-1, CS1, EGFR, FGFR2, AFP, CA125, MUC-1, MAGE, alkaline phosphatase, placental-like 2 (ALPPL2), B-cell maturation antigen (BCMA), green fluorescent protein (GFP), enhanced green fluorescent protein (eGFP), Claudin18.2, PSMA, ROR1, Mesothelin, IL13Ra2, FAP, signal regulatory protein α (SIRPα), TRAC, TCRβ, BCMA, TSHR, EGFRvIII, GD2, GD3, Tn Ag, ROR1, ROR2, GPC1, GPC2, FLT3, FAP, TAG72, CEA, EPCAM, B7H3, KIT, IL-13Ra2, IL-11Ra, PSCA, PRSS21, VEGFR2, LewisY, PDGFR-beta, SSEA-4, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-la, MAGE-Al, legumain, HPV E6,E7, MAGE Al, ETV6-AML, sperm protein 17, XAGE1, Tie 2, MAD-CT-1, MAD-CT-2, Fos-related antigen 1, p53, p53 mutant, KRAS, mutant KRAS, KRAS G12D, prostein, surviving, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, LAIR1, FCAR, LILRA2, CD300LF, CLEC12A, BST2, EMR2, LY75, GPC3, FCRL5, IGLL1, AFP, TRAC, TCRβ, BCMA, TSHR, EGFRvIII, GD2, GD3, Tn Ag, PSMA, ROR1, ROR2, GPC1, GPC2, FLT3, FAP, TAG72, CEA, EPCAM, B7H3, KIT, IL-13Ra2, IL-11Ra, PSCA, PRSS21, VEGFR2, LewisY, PDGFR-beta, SSEA-4, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-la, MAGE-Al, legumain, HPV E6,E7, MAGE Al, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, androgen receptor, cyclin Bi, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, LAIR1, FCAR, LILRA2, CD300LF, CLEC12A, BST2, EMR2, LY75, GPC3, FCRL5, IGLL1, the extracellular portion of the APRIL protein, or any combinations thereof.

In some aspects, the TCR targets AFP, CD19, TRAC, TCRβ, BCMA, CLL-1, CS1, CD38, CD19, TSHR, CD123, CD22, CD30, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, IL-11Ra, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-la, MAGE-Al, legumain, HPV E6,E7, MAGE Al, ETV6-AML, sperm protein 17, XAGE1, Tie 2, MAD-CT-1, MAD-CT-2, Fos-related antigen 1, p53, p53 mutant, KRAS, mutant KRAS, KRAS G12D, prostein, surviving, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, the extracellular portion of the APRIL protein, or any combinations thereof.

A CAR may comprise a costimulatory signaling domain, e.g., to increase signaling potency. See U.S. Pat. Nos. 7,741,465, and 6,319,494, as well as Krause et al. and Finney et al. (supra), Song et al., Blood 119:696-706 (2012); Kalos et al, Sci Transl. Med. 3:95 (2011); Porter et al, N. Engl. J. Med. 365:725-33 (2011), and Gross et al, Annu. Rev. Pharmacol. Toxicol. 56:59-83 (2016). Signals generated through a TCR alone may be insufficient for full activation of a T cell and a secondary or co-stimulatory signal may increase activation. Thus, in some embodiments, a signaling domain further comprises one or more additional signaling domains (e.g., costimulatory signaling domains) that activate one or more immune cell effector functions (e.g., a native immune cell effector function described herein). In some embodiments, a portion of such costimulatory signaling domains may be used, as long as the portion transduces the effector function signal. In some embodiments, a cytoplasmic domain described herein comprises one or more cytoplasmic sequences of a T cell co-receptor (or fragment thereof). Non-limiting examples of such T cell co-receptors comprise CD27, CD28, 4-1BB (CD137), 0X40, CD30, CD40, PD-1, ICOS, lymphocyte function-associated antigen-1 (LFA-1), MYD88, CD2, CD7, LIGHT, NKG2C, B7-H3, and a ligand that binds with CD83. An exemplary costimulatory protein has the amino acid sequence of a costimulatory protein found naturally on T cells, the complete native amino acid sequence of which costimulatory protein is described in NCBI Reference Sequence: NP_006130.1.

In various embodiments, a mechanism of modulating (e.g., decreasing) binding activity of the target antigen is desired, e.g., to minimize or curtail adverse events resulting from binding activity. It may also be desired to comprise an inducible “on” or “accelerator” switch in immune cells. Suitable techniques comprise use of inducible caspase-9 (U.S. Appl. 2011/0286980) or a thymidine kinase, before, after, or at the same time, as the cells are transduced with the CAR construct of the present disclosure. Additional methods for introducing suicide genes and/or “on” switches comprise TALENS, zinc fingers, RNAi, siRNA, shRNA, antisense technology, and other techniques.

In accordance with the present disclosure, on-off or other types of control switch techniques may be incorporated herein. These techniques may comprise use of dimerization domains and optional activators of such domain dimerization, e.g., as disclosed by Wu et al., Science 2015 October; 350(6258):aab4077 utilizing FKBP/Rapalog dimerization systems in certain cells, the contents of which are incorporated by reference herein in their entirety. Additional dimerization technology is described in, e.g., Fegan et al. Chem. Rev. 2010, 110, 3315-3336 as well as U.S. Pat. Nos. 5,830,462; 5,834,266; 5,869,337; and 6,165,787, the contents of each of which is also incorporated by reference herein with respect to dimerization technology. Additional dimerization pairs may comprise cyclosporine-A/cyclophilin, receptor, estrogen/estrogen receptor (optionally using tamoxifen, 4-hydroxytamoxifen, or endoxifen), glucocorticoids/glucocorticoid receptor, tetracycline/tetracycline receptor, and/or vitamin D/vitamin D receptor. Further examples of dimerization technology may be found in e.g., WO 2014/127261, WO 2015/090229, US 2014/0286987, US 2015/0266973, US 2016/0046700, U.S. Pat. No. 8,486,693, US 2014/0171649, and US 2012/0130076, the contents of which are further incorporated by reference herein in their entirety.

In some embodiments, the T cell may comprise a bicistronic CAR. Bicistronic CARs can comprise two CARs that bind different targets and are encoded by a single vector. A bicistronic CAR may comprise a first CAR sequence and a second CAR sequence expressed as a single polypeptide comprising a cleavable linker between the first and second CARs. Non-limiting examples of first and/or second CAR sequences include CD19,CD20, BCMA, CD22, CD70, DLL3, LY6G6D, Claudin 6, GCC, p53R175H, and PRAME. An exemplary cleavable linker is Furin-GSG-T2A (see, e.g., Chng et al. MAbs. 2015 March-April; 7(2): 403-412, which is herein incorporated by reference with respect to cleavable linkers; see also Guedan et al. Mol Ther Methods Clin Dev. 2019 Mar. 15; 12: 145-156, which is incorporated herein by reference with respect to bicistronic CAR design).

In some embodiments, the T cell may comprise a bispecific CAR. In some embodiments, a first binding motif and a second binding motif (e.g., distinct anti-CD20 and anti-CD 19 binding motifs) are both comprised in single bispecific CAR. In such bispecific CARs, a CAR molecule itself may be engineered to recognize more than one antigen. In tandem bispecific CARs, the first and second binding motifs are extracellular and may be characterized as a membrane-proximal binding motif and a membrane-distal binding motif.

Sources for the T cells include, but are not limited to, peripheral blood, bone marrow, or other sources of hematopoietic cells. T cells can be isolated by methods well known in the art, including commercially available isolation methods (see, for example, Rowland-Jones et al., Lymphocytes: A Practical Approach, Oxford University Press, New York (1999), Su et al., Methods Mol. Biol. 806:287-299 (2012); Bluestone et al., Sci. Transl. Med. 7(315) (doi: 10.1126/scitranslmed.aad4134)(2015); Miyara et al., Nat. Rev. Rheumatol. 10:543-551 (2014); Liu et al., J. Exp. Med. 203:1701-1711 (2006); Seddiki et al., J. Exp. Med. 203:1693-1700 (2006); Ukena et al., Exp. Hematol. 39:1152-1160 (2011); Chen et al., J. Immunol. 183:4094-4102 (2009); Putnam et al., Diabetes 58:652-662 (2009); Putnam et al., Am. Tranplant. 13:3010-3020 (2013); Lee et al., Cancer Res. 71:2871-2881 (2011); MacDonald et al., J Clin. Invest. 126:1413-1424 (2016)).

Various known techniques can be employed to isolate or enrich for desired immune cells such as T cells. If a particular type of T cell is to be isolated, various cell surface markers or combinations of markers, including but not limited to, CD3, CD4, CD8, CD34 (for hematopoietic stem and progenitor cells) and the like, can be used to separate the cells, as is well known in the art (see Kearse, T Cell Protocols: Development and Activation, Humana Press, Totowa N.J. (2000); De Libero, T Cell Protocols, Vol. 514 of Methods in Molecular Biology, Humana Press, Totowa N.J. (2009)) Negative selection methods can be used to remove cells that are not the desired immune cells. Additionally, positive selection methods can be used to isolate or enrich for desired T cells. In some instances, a combination of both positive and negative selection methods can be used.

### Pharmaceutical Compositions

In some embodiments, the recombinant nucleic acid constructs and/or recombinant nucleic acids of the disclosure and recombinant cells of the disclosure can be incorporated into compositions, including pharmaceutical compositions. Such compositions generally include the recombinant nucleic acids, recombinant polypeptides, and/or recombinant cells of the disclosure, and a pharmaceutically acceptable excipient, e.g., carrier.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Systemic administration of the subject recombinant nucleic acid constructs and/or recombinant nucleic acids and recombinant cells of the disclosure can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In some embodiments, the recombinant nucleic acid constructs and/or recombinant nucleic acids and recombinant cells of the disclosure can also be administered by transfection or infection using methods known in the art, including but not limited to the methods described in McCaffrey et al. (Nature 418:6893, 2002), Xia et al. (Nature Biotechnol. 20: 1006-1010, 2002), or Putnam (Am. J. Health Syst. Pharm. 53: 151-160, 1996, erratum at Am. J. Health Syst. Pharm. 53:325, 1996).

In some embodiments, the recombinant nucleic acid constructs and/or recombinant nucleic acids and recombinant cells of the disclosure are prepared with carriers that will protect the recombinant polypeptides against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811. As described in greater detail below, the polypeptides of the present disclosure may also be modified to achieve extended duration of action such as by PEGylation, acylation, Fc fusions, linkage to molecules such as albumin, etc. In some embodiments, the recombinant polypeptides can be further modified to prolong their half-life in vivo and/or ex vivo. Non-limiting examples of known strategies and methodologies suitable for modifying the recombinant polypeptides of the disclosure include (1) chemical modification of a recombinant polypeptide described herein with highly soluble macromolecules such as polyethylene glycol (“PEG”) which prevents the recombinant polypeptides from contacting with proteases; and (2) covalently linking or conjugating a recombinant polypeptide described herein with a stable protein such as, for example, albumin. Accordingly, in some embodiments, the polypeptides of the disclosure can be fused to a stable protein, such as, albumin. For example, human albumin is known as one of the most effective proteins for enhancing the stability of polypeptides fused thereto and there are many such fusion proteins reported.

In some embodiments, the pharmaceutical compositions of the disclosure include one or more PEGEylation reagents. In some embodiments, the PEGylation reagent is selected from methoxy polyethylene glycol-succinimidyl propionate (mPEG-SPA), mPEG-succinimidyl butyrate (mPEG-SBA), mPEG-succinimidyl succinate (mPEG-SS), mPEG-succinimidyl carbonate (mPEG-SC), mPEG-Succinimidyl Glutarate (mPEG-SG), mPEG-N-hydroxyl-succinimide (mPEG-NHS), mPEG-tresylate and mPEG-aldehyde. In some embodiments, the PEGylation reagent is polyethylene glycol. In some embodiments, the PEGylation reagent is polyethylene glycol with an average molecular weight of 20 kD covalently bound to the N-terminal methionine residue of the recombinant polypeptides of the disclosure., or about 80 kD covalently bound to the N-terminal methionine residue of the polypeptides of the disclosure. In some embodiments, the PEGylation reagent is polyethylene glycol with an average molecular weight of about 40 kD covalently bound to the N-terminal methionine residue of the polypeptides of the disclosure.

Accordingly, in some embodiments, recombinant nucleic acid constructs and/or recombinant nucleic acids and recombinant cells of the disclosure are chemically modified with one or more polyethylene glycol moieties, e.g., PEGylated; or with similar modifications, e.g. PASylated. In some embodiments, the PEG molecule or PAS molecule is conjugated to one or more amino acid side chains of the disclosed recombinant polypeptide. In some embodiments, the PEGylated or PASylated polypeptide contains a PEG or PAS moiety on only one amino acid. In other embodiments, the PEGylated or PASylated polypeptide contains a PEG or PAS moiety on two or more amino acids, e.g., attached to two or more, five or more, ten or more, fifteen or more, or twenty or more different amino acid residues. In some embodiments, the PEG or PAS chain is 2000, greater than 2000, 5000, greater than 5,000, 10,000, greater than 10,000, greater than 10,000, 20,000, greater than 20,000, and 30,000 Da. The PASylated polypeptide may be coupled directly to PEG or PAS (e.g., without a linking group) through an amino group, a sulfhydryl group, a hydroxyl group, or a carboxyl group. In some embodiments, the recombinant polypeptide of the disclosure is covalently bound to a polyethylene glycol with an average molecular weight of 20,000 Daltons. In some embodiments, the recombinant polypeptide of the disclosure is covalently bound to a polyethylene glycol with an average molecular weight ranging from about 1 kD to about 200 kD such as, e.g., about 10 kD to about 150 kD, about 50 kD to about 100 kD, about 5 kD to about 100 kD, about 20 kD to about 80 kD, about 30 kD to about 70 kD, about 40 kD to about 60 kD, about 50 kD to about 100 kD, about 100 kD to about 200 kD, or about 1 150 kD to about 200 kD. In some embodiments, the recombinant polypeptide of the disclosure is covalently bound to a polyethylene glycol with an average molecular weight of about 5 kD, about 10 kD, about 20 kD, about 30 kD, about 40 kD, about 50 kD, about 60 kD, about 70 kD, or about 80 kD. In some embodiments, the recombinant polypeptide of the disclosure is covalently bound to a polyethylene glycol with an average molecular weight of about 40 kD

### Methods of The Disclosure

The disclosure also relates to methods including the polypeptides, recombinant nucleic acid constructs and/or recombinant nucleic acids or the cells of the disclosure.

### Methods for Identifying a Mutation Useful for Improving T Cell Therapy

In an aspect, the disclosure relates to methods for identifying a mutation useful (beneficial) for improving T cell therapy (e.g., as described in Example 1). The mutation can be identified from genomic sequencing data of T cell lymphomas or from clonal T cells or other T cells. The methods can include applying a statistical test to determine a mutation that occurs more often than expected by chance in a hotspot region of a coding sequence. As used herein, a hotspot region can be a segment of DNA that is prone to genetic alteration. In some embodiments, the hotspot region is in a coding sequence of a gene. In some embodiments, the statistical tests employ a binomial distribution. In some embodiments, the statistical tests employ Chi square analysis or any other multivariate analysis.

The binomial distributions can be employed across gene lengths in the entire genome of a cell. The statistical tests can employ various false discovery rates. In some embodiments, the discovery rate can be 0.05. In some embodiments, it can be controlled for by the Benjamini-Hochberg algorithm. In some embodiments, heterogeneity due to transcription couple repair can be accounted for.

In some embodiments, a mutation useful or beneficial for improving T cell therapy, can be identified by selecting a mutation that has occurred in patients. In some embodiments, a mutation can be identified by performing a statistical analysis of genomic sequencing data of clonal T cells, including T cell lymphomas, from public and/or private databases. In some embodiments, a mutation is identified by using binomial distributions across gene lengths in the entire genome and a false discovery rate of 0.05 controlled for by the Benjamini-Hochberg algorithm. In some embodiments, the mutation is identified in hotspot regions where the mutation occurred more often than expected by chance alone in the hotspot regions. In some embodiments, a mutation is identified by permuting the background rate of mutations across the genome accounting for heterogeneity due to transcription couple repair thereby identifying genes that harbor mutations that occur more often than expected by chance using the permuted background rate of mutations.

The identified mutation can be a mutation that increases proliferation of therapeutic T cells, or alters effector function, or resists T cell dysfunction, and/or T cell enhances growth, or reduces T cell exhaustion or promotes in vivo persistence of T cells. Increasing proliferation of therapeutic T cells can include clonal expansion, increase in replication rate of T cells and/or increase in the number of T cells. The mutation can be any mutation listed in Table 1.

T-cell effector functions can involve the interaction of an armed effector T cell with a target cell displaying specific antigen. The effector proteins released by these T cells are focused on the appropriate target cell by mechanisms that are activated by recognition of antigen on the target cell.

In some embodiments, the method for identifying a mutation useful (beneficial) for improving T cell therapy includes various steps such as a) identifying mutations from a clonal T cell genomic sequencing database; b) identifying a frequency of occurrence of the mutations; and c) applying a statistical test to identify a significant difference in a hotspot genomic region where a mutation occurs more often than expected by chance, thereby identifying a mutation in the hotspot region that is capable of improving T cell therapy. The mutation can improve T cell therapy by increasing proliferation, altering effector function, resisting T cell dysfunction, and/or enhancing growth, of a therapeutic T cell including the mutation, in a tumor. The mutation can promote positive T cell selection and/or T cell clonal outgrowth. The mutation can be any mutation listed in Table 1.

### Methods for Preparing T Cells for Use in Cell Therapy

The disclosure also provides methods for preparing a T cell for use in cell therapy. The T cell can further comprise a CAR, a TCR, and a transcriptional receptor. The T cell can be an NK cell or a tumor infiltrating lymphocyte from a patient with cancer. Preparing the cell can include introducing (e.g., by transducing) into a cell any one or more of the recombinant nucleic acid constructs and/or recombinant nucleic acids of the disclosure. Preparing the cell can include introducing (e.g., by transducing) into a cell any one or more vectors comprising any one or more of the recombinant nucleic acid constructs and/or recombinant nucleic acids of the disclosure. Preparing the cell can include expressing in the cell any one or more of the polypeptides of the disclosure. The cell can be genetically modified for expression of the polypeptide. The cell can further comprise expression of an engineered immune receptor that binds to a target in a tumor cell.

The cells can be transduced with a recombinant nucleic acid construct that can alter T cell signaling via the NFAT pathway, NF-κB pathway, AP-1 pathway, JAK/STAT pathway, RAS/MEK/ERK, phospholipase gamma signaling or other T cell signaling pathways.

The methods of preparing a T cell according to the disclosure can include transducing T cells with recombinant nucleic acid constructs and/or recombinant nucleic acids capable of enhancing or promoting or improving or reducing or regulating or modulating the pathway or activating or increasing or suppressing or inhibiting or other means of changing T cell signaling. The methods of preparing a T cell according to the disclosure can include transducing cells with recombinant nucleic acid constructs and/or recombinant nucleic acids that can alter CARD11-BCL10-MALT1 complex signaling, co-stimulatory molecule signaling, and cytokine production and/or transcription factor activity in T cells.

In an embodiment, the method of preparing a T cell for use in a cell therapy includes transducing the T cell with a recombinant nucleic acid construct and/or recombinant nucleic acid having a mutation capable of altering (i) T cell signaling through NFAT, NF-κB and/or AP-1 pathways, (ii) cytokine production, and/or (iii) in vivo persistence of T cells in tumors.

In some embodiments of the methods of preparing a T cell for use in a cell therapy includes polypeptides and/or recombinant nucleic constructs and/or recombinant nucleic acids that can alter in vivo persistence in tumors of therapeutic T cells including the mutation. In some embodiments, the recombinant nucleic acid constructs and/or recombinant nucleic acids of the disclosure can alter the therapeutic efficacy, decrease T cell exhaustion, increase proliferative capacity, enhance anti-tumor effect, enhance the replicative lifespan, decrease replicative senescence, and enhance ability to kill, enhance the fitness of engineered T cells and/or other functions or activities of T cells.

In some embodiments, the polypeptides or recombinant nucleic acid constructs and/or recombinant nucleic acids of the disclosure can alter the in vivo persistence in tumors, in vivo accumulation in tumors, or the other functions or activities of T cells described herein, through a mutation in one or more of the forty one genes listed in Table 1 or FIG. 2A. In some embodiments, the genes include caspase recruitment domain family member 11 (CARD11). In some embodiments, the genes include capping protein regulator and myosin 1 linker 2 (CARMIL2), mucosa-associated lymphoid tissue lymphoma translocation protein 1 (MALT1), B-cell lymphoma 6 (BCL6), B-cell lymphoma 10 (BCL10) and MYCN. The gene can be a signal transducer and activator of transcription gene (STAT) including STAT3, STAT5B. The gene can be a janus kinase gene such as JAK1, JAK2, or JAK3 gene. The gene can be v-raf murine sarcoma viral oncogene homolog B1 (BRAF). The gene can be RAS Guanyl Releasing Protein 1 (RASGRP1). The gene can be Tumor necrosis factor receptor superfamily member 1B (TNFRSF1B). The gene can be phospholipase C gamma 1 (PLCG1) gene. The gene can be nuclear factor kappa light chain enhancer of activated B cells (NF-κB1), or (NF-1B2) or JunB Proto-Oncogene, AP-1 Transcription Factor Subunit (JUNB) gene.

### Methods for Treatmenting a Subject

The disclosure also provides methods of treating a subject in need of cell therapy, comprising administering to the subject a cell as described herein or a T-cell prepared by any of the methods described herein. The disclosure also provides methods for enhancing the in vivo persistence of a T cell (e.g., a therapeutic T cell) in a subject in need thereof, by administering to the subject a therapeutically effective amount of the T cells of the disclosure. The T cells can include any of the recombinant nucleic acid constructs and/or recombinant nucleic acids of the disclosure.

This administering step can be accomplished using any method of implantation delivery in the art. For example, the recombinant cells of the disclosure can be infused directly in the individual's bloodstream or otherwise administered to the individual.

In some embodiments, the methods disclosed herein include administering which term is used interchangeably with the terms “introducing,” implanting,” and “transplanting,” recombinant cells into an individual, by a method or route that results in at least partial localization of the introduced cells at a desired site such that a desired effect(s) is/are produced. The recombinant cells or their differentiated progeny can be administered by any appropriate route that results in delivery to a desired location in the individual where at least a portion of the administered cells or components of the cells remain viable. The period of viability of the cells after administration to an individual can be as short as a few hours, e.g., twenty-four hours, to a few days, to as long as several years, or even the lifetime of the individual, i.e., long-term engraftment.

When provided prophylactically, the recombinant cells described herein can be administered to an individual in advance of any symptom of a disease or condition to be treated. Accordingly, in some embodiments the prophylactic administration of a recombinant cell population prevents the occurrence of symptoms of the disease or condition.

When provided therapeutically in some embodiments, recombinant cells are provided at (or after) the onset of a symptom or indication of a disease or condition, e.g., upon the onset of disease or condition.

For use in the various embodiments described herein, an effective amount of recombinant cells as disclosed herein, can be at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. The recombinant cells can be derived from one or more donors or can be obtained from an autologous source. In some embodiments, the recombinant cells are expanded in culture prior to administration to an individual in need thereof.

In some embodiments, the delivery of a recombinant cell composition (e.g., a composition including a plurality of recombinant cells according to any of the cells described herein) into an individual by a method or route results in at least partial localization of the cell composition at a desired site. A composition including recombinant cells can be administered by any appropriate route that results in effective treatment in the individual, e.g., administration results in delivery to a desired location in the individual where at least a portion of the composition delivered, e.g., at least 1×10⁴ cells, is delivered to the desired site for a period of time. Modes of administration include injection, infusion, and instillation. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. In some embodiments, the route is intravenous. For the delivery of cells, delivery by injection or infusion is a preferred mode of administration.

In some embodiments, the recombinant cells are administered systemically, e.g., via infusion or injection. For example, a population of recombinant cells are administered other than directly into a target site, tissue, or organ, such that it enters the individual's circulatory system and, thus, is subject to metabolism and other similar biological processes.

The efficacy of a treatment including any of the compositions provided herein for the treatment of a disease or condition can be determined by a skilled clinician. However, one skilled in the art will appreciate that a treatment is considered effective if any one or all of the signs or symptoms or markers of disease are improved or ameliorated. Efficacy can also be measured by failure of an individual to worsen as assessed by decreased hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

As discussed above, a therapeutically effective amount includes an amount of a therapeutic composition that is sufficient to promote a particular beneficial effect when administered to an individual, such as one who has, is suspected of having, or is at risk for a disease. In some embodiments, an effective amount includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

In some embodiments of the disclosed methods, the individual is a mammal. In some embodiments, the mammal is a human. In some embodiments, the individual has or is suspected of having a disease associated with inhibition of cell signaling mediated by a cell surface ligand or antigen. The diseases suitable for being treated by the compositions and methods of the disclosure include, but are not limited to, cancers, autoimmune diseases, inflammatory diseases, and infectious diseases. In some embodiments, the disease is a cancer or a chronic infection.

Methods for CAR design, delivery and expression in T cells, and the manufacturing of clinical-grade CAR-T cell populations are known in the art. See, for example, Lee et al., Clin Cancer Res (2012) 18(10):2780-90, hereby incorporated by reference in its entirety. For example, the engineered CARs may be introduced into T cells using retroviruses, which efficiently and stably integrate a nucleic acid sequence encoding the chimeric antigen receptor into the target cell genome.

Other methods known in the art include, but are not limited to, lentiviral transduction, transposon-based systems, direct RNA transfection, and CRISPR/Cas systems (e.g., type I, type II, or type III systems using a suitable Cas protein such Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, Cas12a (Cpf1), Cas13a (C2c2), Cas13b, Cas13d, CasF, CasG, CasH, Csy1, Csy2, Csy3, Cse1 (or CasA), Cse2 (or CasB), Cse3 (or CasE), CasX, CasY, Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966, etc.).

In some embodiments, a recombinant adeno-associated virus (AAV) vector can be used for delivery. Techniques to produce rAAV particles, in which an AAV genome to be packaged that includes the polynucleotide to be delivered, rep and cap genes, and helper virus functions are provided to a cell are standard in the art. Production of rAAV requires that the following components are present within a single cell (denoted herein as a packaging cell): a rAAV genome, AAV rep and cap genes separate from (e.g., not in) the rAAV genome, and helper virus functions. The AAV rep and cap genes can be from any AAV serotype for which recombinant virus can be derived, and can be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13 and AAV rh.74. Production of pseudotyped rAAV is disclosed in, for example, international patent application publication number WO 01/83692.

The CAR-T cells, once they have been expanded ex vivo in response to, for example, an autoimmune disease antigen, can be reinfused into the subject in a therapeutically effective amount.

The precise amount of CAR T cells to be administered can be determined by a physician with consideration of individual differences in age, weight, extent of disease and condition of the subject.

Administration of T cell therapies may be defined by number of total cells per infusion or number of cells per kilogram of body weight, especially for pediatric subjects (e.g., patients). As T cells replicate and expand after transfer, the administered cell dose may not resemble the final steady-state number of cells. In some embodiments, a pharmaceutical composition including the CAR T cells of the present disclosure may be administered at a dosage of 10⁴ to 10¹⁰ total cells. In another embodiment, a pharmaceutical composition including the CAR T cells of the present disclosure may be administered at a dosage of 10³ to 10⁸ cells/kg body weight, including all integer values within those ranges.

Compositions including the CAR T cells of the present disclosure may also be administered multiple times at these dosages. The cells can be administered by using infusion techniques that are known in the art (see, for example, Rosenberg et al., New Engl J Med, (1988) 319:1676). The optimal dosage and treatment regimen for a particular subject can be determined by one skilled in the art by monitoring the subject for signs of disease and adjusting the treatment accordingly.

In some embodiments, administration of any of the compositions embodied herein, for the treatment of, for example, an autoimmune or inflammatory disease, can be combined with other cell-based therapies, for example, stem cells, antigen presenting cells, pancreatic islets etc.

The composition of the present disclosure may be prepared in a manner known in the art and in a manner suitable for parenteral administration to mammals, particularly humans, including a therapeutically effective amount of the composition alone, with one or more pharmaceutically acceptable carriers or diluents.

The term “pharmaceutically acceptable carrier” as used herein means any suitable carriers, diluents or excipients. These include all aqueous and non-aqueous isotonic sterile injection solutions, which may contain anti-oxidants, buffers and solutes, which render the composition isotonic with the blood of the intended recipient; aqueous and non-aqueous sterile suspensions, which may include suspending agents and thickening agents, dispersion media, antifungal and antibacterial agents, isotonic and absorption agents and the like. It will be understood that compositions of the present disclosure may also include other supplementary physiologically active agents.

The carrier must be pharmaceutically “acceptable” in the sense of being compatible with the other ingredients of the composition and not injurious to the subject. Compositions include those suitable for parenteral administration, including subcutaneous, intramuscular, intravenous and intradermal administration. The compositions may conveniently be presented in unit dosage form and may be prepared by any method well known in the art of pharmacy. Such methods include preparing the carrier for association with the CAR-T cells. In general, the compositions are prepared by uniformly and intimately bringing into association any active ingredients with liquid carriers.

In some embodiments, the composition is suitable for parenteral administration. In another embodiment, the composition is suitable for intravenous administration.

Compositions suitable for parenteral administration include aqueous and nonaqueous isotonic sterile injection solutions which may contain anti-oxidants, buffers, bactericides and solutes, which render the composition isotonic with the blood of the intended recipient; and aqueous and non-aqueous sterile suspensions which may include suspending agents and thickening agents.

Enhancing the in vivo persistence of a T cell in a subject can be enhancing the fitness or enhancing the function and/or enhancing the efficacy of the therapeutic T cells. The enhancing can be measured by determining the accumulation of the number of T cell in a tumor (for example as described in Example 13 below).

In some embodiments, in vivo persistence can be by promoting the intra-tumoral increase of effector cytokines. In some embodiments, the in vivo persistence can be by increasing the expression of the stemness-associated transcription factor TCF1 in TILs. In some embodiments, the in vivo persistence can be by increasing the expression of TNF-α, IFN-7, and/or IL-2.

In some embodiments, enhancing the in vivo persistence can be measured by measuring differences in gene expression. In some embodiments, the genes include but are not limited to activation markers (IL2RA, CD69), cytotoxic and effector molecules (IFNG, TNF, IL4, IL5, IL13, GZMA, GZMB), chemokines (CCL4, CCL20, CCL8), and co-stimulatory molecules (ICOS, OX40, 4-1BB, GITR).

In some embodiments, the subject has cancer or an autoimmune disease. In some embodiments, the cancer can be a solid tumor. In some embodiments, the cancer can be a hematological cancer. In some embodiments, the cancer expresses a tumor-associated antigen, as described herein. In some embodiments, the cancer expresses DLL3, LY6G6D, Claudin 6, GCC, p53R175H, and/or PRAME.

Exemplary solid tumors include, without limitation, small cell lung cancer, colorectal cancer, testicular cancer, ovarian cancer, or melanoma, lymphoma, leukemia, multiple myeloma, prostate cancer, breast cancer, non-small cell lung cancer, gastric cancer, esophageal cancer, liver cancer, kidney cancer, head & neck cancer, glioblastoma, neuroblastoma, soft tissue sarcoma, uterine cancer, brain cancer, skin cancer, renal cancer, bladder cancer, pancreatic cancer, thyroid cancer, eye cancer, gastrointestinal cancer, carcinoma, and sarcoma.

In some embodiments, the method of treatment does not comprise administration of lymphodepletive agents within 7 days prior to administration of the T cell therapy. Non-limiting examples of lymphodepletive agents include cyclophosphamide, fludarabine, and/or bendamustine within 7 days prior to administration of the T cell therapy. In some embodiments, the method of treatment does not comprise administration of at least 600,000 IU/kg of IL-2 every 8 hours. In some embodiments, the method of treatment does not comprise a checkpoint therapy which blocks PD-1 or CTLA-4 signaling.

In some embodiments, the method of treatment comprises a cell with reduced exhaustion, increased proliferative capacity, enhanced replicative lifespan, decreased replicative senescence, enhanced anti-tumor effect, reduced dysfunction, enhanced persistence, and/or increase intratumoral presence in vivo. In some embodiments, the method of treatment comprises a cell with increased or decreased signaling through the CARD11-BCL10-MALT1 complex, NF-κB, AP-1, NFAT, JAK/STAT, and/or MEK/ERK pathways.

The cells of the methods of the disclosure can be regulatory (Treg), natural killer (NK) cells, a gamma delta T cell, an invariant iNKT cell, a macrophage, a monocyte, TILs or engineered T cells. In some embodiments, the engineered T cells can express a recombinant TCR or a CAR or a transcriptional receptor.

The engineered T cells can be autologous or allogeneic/non-autologous to the subject to which they are administered in the methods of the disclosure. For example, autologous cells can be isolated from the subject to which the T cells are to be administered. The autologous cells can be isolated from the subject to which the engineered cells recombinantly expressing a CAR or a transcriptional receptor are to be administered. Optionally, the cells can be obtained by leukapheresis, where leukocytes are selectively removed from withdrawn blood, engineered and made recombinant, and then retransfused into the donor. Alternatively, allogeneic cells from an allogeneic/non-autologous donor that is not the subject can be used. In the case of an allogeneic/non-autologous donor, the cells are typed and matched for human leukocyte antigen (HLA) to determine an appropriate level of compatibility, as is well known in the art. For both autologous and allogeneic/cells, methods for genetic manipulation and/or administration to a subject are well known in the art. In some situations, the cells can be optionally preserved (e.g., cryopreserved) until ready to be used.

Procedures for separation of cells include, but are not limited to, flow cytometry, affinity chromatography, density gradient centrifugation, magnetic separation with antibody-coated magnetic beads, conjugation to particles that modify cell density, cytotoxic agents joined to or used in conjunction with a monoclonal antibody (mAb) or any other appropriate technique.

In some embodiments, the isolated T cells are genetically engineered ex vivo for recombinant expression of a transgene. In some embodiments, isolated T cells are genetically engineered ex vivo for recombinant expression of a CAR or a transcriptional receptor as described in more details supra. In some embodiments, the cells can be genetically engineered for recombinant expression. Methods suitable for genetically engineering cells for recombinant expression are well known in the art.

### Systems and Kits

Systems or kits of the present disclosure include one or more of any of the recombinant nucleic acids, recombinant cells, or pharmaceutical compositions disclosed herein as well as syringes (including pre-filled syringes) and/or catheters (including pre-filled syringes) used to administer any of the recombinant nucleic acids, recombinant cells, or pharmaceutical compositions to a subject. The kits also include written instructions for using of any of the recombinant nucleic acids, recombinant cells, or pharmaceutical compositions disclosed herein as well as syringes and/or catheters for use with their administration.

Any of the above-described systems and kits can further include one or more additional reagents, where such additional reagents can be selected from: dilution buffers; reconstitution solutions, wash buffers, control reagents, control expression vectors, negative control polypeptides, positive control polypeptides, reagents for in vitro production of the chimeric receptor polypeptides.

In some embodiments, the components of a system or kit can be in separate containers. In some other embodiments, the components of a system or kit can be combined in a single container.

In some embodiments, a system or kit can further include instructions for using the components of the kit to practice the methods. The instructions for practicing the methods are generally recorded on a suitable recording medium. For example, the instructions can be printed on a substrate, such as paper or plastic, etc. The instructions can be present in the kits as a package insert, in the labeling of the container of the kit or components thereof (i.e., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the inventors/disclosers reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

### EXAMPLES

The practice of the present disclosure will employ, unless otherwise indicated, conventional techniques of molecular biology, microbiology, cell biology, biochemistry, nucleic acid chemistry, and immunology, which are well known to those skilled in the art. Such techniques are explained fully in the literature cited above.

Additional embodiments are disclosed in further detail in the following examples, which are provided by way of illustration and are not in any way intended to limit the scope of this disclosure or the claims.

### Example 1

### Identification of Mutations

This Example describes the design and implementation of a statistical test to identify mutations for improving T cell therapy by altering signaling pathways, reducing T cell exhaustion, increasing therapeutic T cell proliferation, altering T cell effector function, resisting T cell dysfunction, enhancing growth in harsh tumor microenvironment, increasing in vivo persistence of therapeutic T cells and/or by other means.

In order to identify a mutation useful or beneficial for improving T cell therapy, the inventors/disclosers either 1—selected mutations that occurred in patients or 2—performed a statistical analysis of genomic sequencing data of clonal T cells, including T cell lymphomas, and have identified, from public and private databases, using binomial distributions across gene lengths in the entire genome and a false discovery rate of 0.05 controlled for by the Benjamini-Hochberg algorithm, hotspot regions and mutations that occur more often than expected by chance alone in the hotspot regions. In particular, the inventors/disclosers permuted the background rate of mutations across the genome accounting for heterogeneity due to transcription couple repair. Then they identified genes that harbor mutations that occur more often than expected by chance assuming the background rate of mutations.

Using the methods, the inventors/disclosers identified and cloned 62 point mutations (encoding non-synonymous amino acid substitutions and putative gain-of-function truncating mutations) in 40 different genes. In addition, the inventors/disclosers identified 10 gene fusions.

### Example 2

### Library Construction and In Vitro Mutation Screening

This Example describes the design and construction of the library of mutations shown in Table 1 and the screening of the constructs in order to identify the constructs that could improve in vivo persistence of human CAR T cells.

The inventors/disclosers cloned the 62 identified point mutations and the 10 fusion polypeptides. For each point mutation construct, a wild-type control of the same gene was generated to control for the effects of overexpression of the wild-type form of the gene. Five control constructs were also cloned. These targets were cloned into a barcoded lentiviral construct to enable pooled screening. In total, the library for T cell lymphoma mutation screening included 117 unique constructs (FIGS. 1 and 2A-D).

Wildtype genes of the mutation library were ordered as plasmids through DNASU, and point mutations were introduced through PCR site directed mutagenesis. For some mutated genes the genes were synthesized by Twist Biosciences (South San Francisco, CA). Wild type or mutated gene fragments were cloned into a modified pHR′SIN:CSW vector containing a PGK promoter followed by a T2A self-cleaving sequence, a unique barcode and the fluorescent tag mCherry used to identify transduced cells. Wild type or mutant gene fragments were cloned via a Sbf1 site in the multiple cloning site 3′ to the PGK promoter sequence. All constructs were cloned via Infusion cloning (Clontech #ST0345) or Gibson assembly.

Intracellular domains containing the appropriate costimulatory domain, and CD3zeta domain were synthesized as synthesized by Twist. Receptors were built by fusing the CD19 scFv to the corresponding receptor scaffold and intracellular tail. All receptors contained an n-terminal CD8a signal peptide (MALPVTALLLPLALLLHAARP SEQ ID NO: 261) for membrane targeting and a flag-tag (DYKDDDDK SEQ ID NO: 262) for easy determination of surface expression with α-flag PE (Biolegend 637310). In some cases the receptors additionally contained a T2A self-cleaving sequence followed by a tNGFR sequence, used in downstream applications for T cell isolations. The receptors were cloned into a modified pHR′SIN:CSW vector containing a PGK promoter for all primary T cell experiments.

Triple reporter Jurkat cells (a human T cell line) that stably express NFAT-eGFP, NF-kB-eCFP, and AP-1-iRFP fluorescent protein reporter constructs were generated.

These triple reporter cells were then transduced to express a CD19-CD28z or CD19-BBz CAR, which led to CD19 antigen dependent signaling and IL-2 production.

The effects of most of the mutations had not been fully characterized in the context of T cell signaling and effector function. To elucidate their functions, the inventors/disclosers transduced the triple reporter line with each construct in the library. Collectively, the reporter cells enabled elucidation of the mutations' effects on biochemical signaling pathways, the effects of antigen and the evaluation of effector cytokine production. A schematic of the in vitro and in vivo screens of the mutations to uncover effects of the mutations on T cell signaling pathways using triple reporter Jurkat cells (a human T cell line) stably expressing NFAT-eGFP, NF-kB-eCFP and AP-1-iRFP fluorescent protein reporter constructs is illustrated in FIG. 1.

To screen the effects of T cell lymphoma mutations on CAR signaling, triple reporter CAR cells were transduced with a CD19-CD28z or a CD19-BBz chimeric antigen receptor (CAR). Then co-cultured with K652 or K562-CD19 cells, and NFAT, NF-κB, and AP-1 reporter activity was determined by flow cytometry. As a readout of effector functions, supernatants from the K562-CD19 condition were collected and analyzed for IL-2 by ELISA. Each screen was performed twice with independent transductions and was highly reproducible across biological replicates. In vitro screens uncovered numerous mutations with significant impacts on CAR signaling and cytokine production (FIG. 2, Table 3-5). For the CD19-BBz CAR screen, PD-1 levels were assessed by flow cytometry.

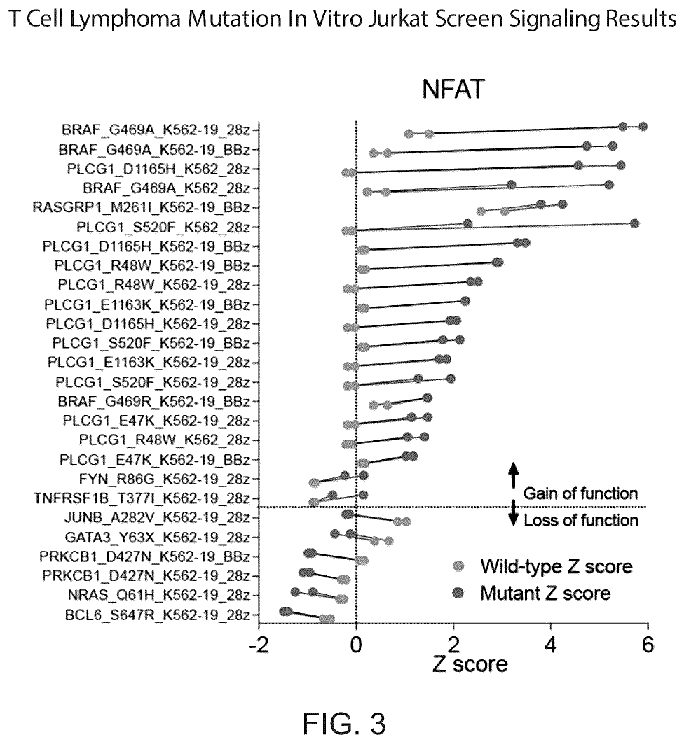

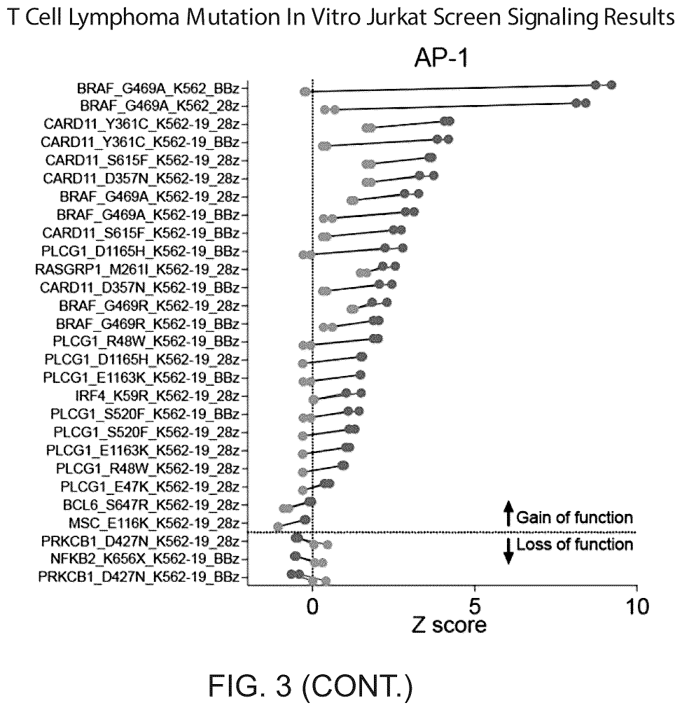

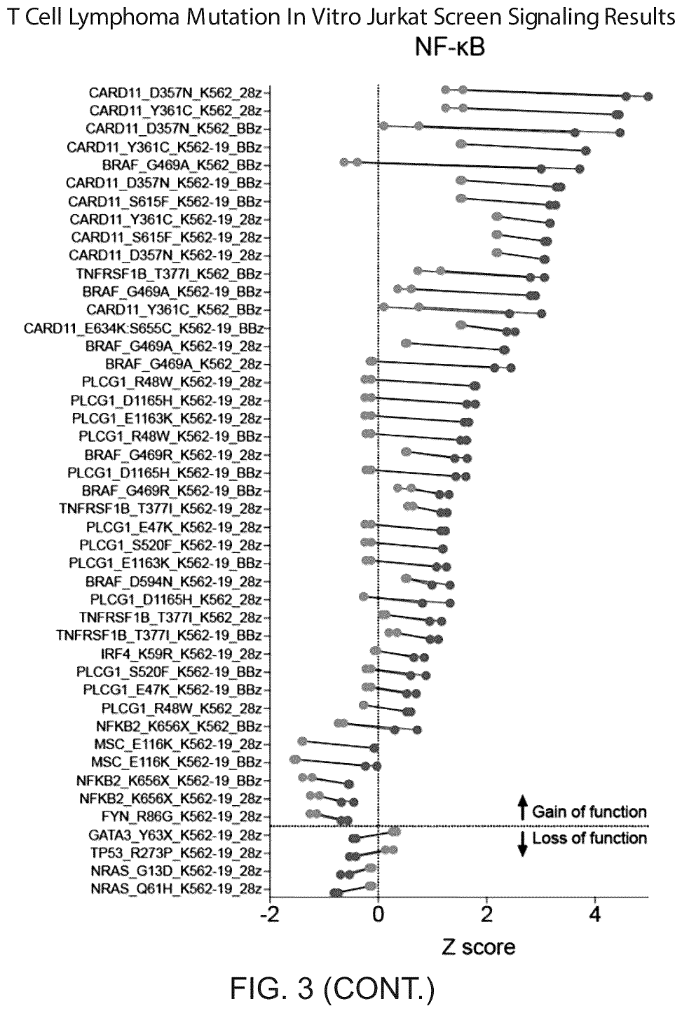

Mutations altered signaling in ways that were unachievable via expression of the wild-type form of the gene. Twenty-five point mutation constructs showed significant differences versus their wild-type counterparts, demonstrating substantial increases or decreases of signaling endowed by the mutations not attributable to overexpression of the wild-type genes (FIG. 3). Therefore, this mutation library allows for the alteration of T cell signaling in ways that are not possible with overexpression of wild-type genes. This feature of the mutation library differentiates it from approaches were expression of wild-type genes are enhanced or elevated.

In vitro screening uncovered numerous mutations with significant impacts on CAR signaling and cytokine production. These screens were highly reproducible across two biological replicates. Similar effects were observed when mutations were paired with either the CD19-CD28z CAR or CD19-BBz CAR. Mutations previously reported to upregulate TCR dependent signaling (e.g. PLCG1) had effects that were consistent with these previous findings, suggesting our assay effectively captures known positives. In addition, expression of the negative control construct DGKZ, which encodes diacyl glycerol kinase zeta, a known inhibitor of T cell receptor signaling, significantly reduced CAR dependent signaling, as expected.

The mutation constructs showed a significant degree of antigenic specificity. For both the CD19-CD28z CAR and the CD19-BBz CAR, mutation constructs showed significantly more effects upon antigenic stimulation than in the absence of antigen (FIG. 2E).

The mutations produced a striking diversity of effects on TCR-dependent signaling (FIG. 2F). For the CD19-BBz CAR, a total of 10 different combinations of signaling pathway up- or down-regulation induced by mutations were observed. Some of these modifications may be desirable. For example, imbalances between NFAT and AP-1 signaling may contribute to T cell exhaustion. Therefore, mutations that increase AP-1 but not NFAT may be beneficial to T cell therapies. 25 mutation constructs showed significant differences versus their wild-type counterparts, demonstrating substantial increases or decreases of signaling endowed by the mutations not attributable to overexpression of the wild-type genes (FIG. 3).

Moreover, these mutations provide effect sizes unachievable with changes in gene expression alone, enabling tunable changes over wide dynamic ranges. As an example of this tunability, AP-1 reporter expression could be tuned down or up over a range of 0.4 fold to nearly 3 fold the levels induced by CAR activation in controls (FIG. 2G).

### Example 3

### In Vivo Mutation Screening

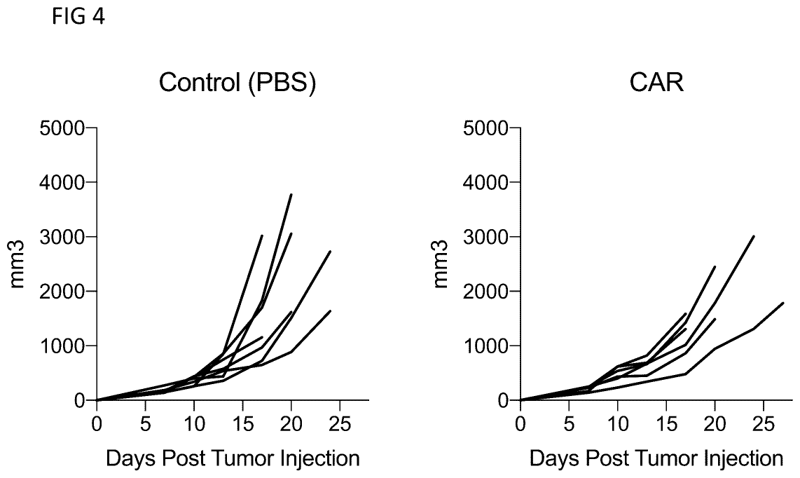

Next, the inventors/disclosers systematically screened T cell lymphoma mutations to identify constructs which could improve in vivo persistence of human CAR-T cells as follows. Primary human CD3+ T cells were co-transduced with a CD19-BBz CAR and T cell lymphoma mutation constructs. Cells expressing both the CAR and the mutation constructs were pooled, sorted, and then injected into immunodeficient mice bearing subcutaneous CD19-K562 tumors (FIG. 1). A sample of this pre-injection pooled library was taken for sequencing of barcodes. Barcode frequency in tumors were compared to the pre-injection T cell pool to determine constructs which were depleted or enriched in vivo. The single most depleted construct in the library was PDCD1, which encodes the co-inhibitory receptor and immunotherapy target PD-1, suggesting that the in vivo screen was able to identify therapeutically relevant targets (FIG. 5A). The K562 subcutaneous xenograft model generally shows poor T cell infiltration and persistence, and a lack of anti-tumor efficacy of CAR-T cells (FIG. 4). This model was employed to identify constructs which enable T cells to overcome the harsh solid tumor microenvironment that limits therapeutic T cell efficacy. 1×10⁶ CD19-K562s were injected subcutaneously to NSG mice on day 0, followed by intravenous injection of 5×10⁶ CD19-BBz CAR cell or PBS vector control cells on Day 4. These data show that CD19-BBz CAR cells control CD19-K562 growth poorly, suggesting T cell function is limited in this model and providing rationale for using this model to screen for mutation constructs which improve T cell function. Constructs which improve the infiltration and/or persistence in this model could be valuable for improving the anti-tumor efficacy of cell therapies against solid tumors.

In vivo screening identified 35 mutation constructs with positive log 2 fold changes in vivo (FIG. 5A). Positive log 2 fold changes indicate mutations, which were enriched in tumors compared to pre-injection samples. Enrichment in tumors suggests these constructs enable T cells to accumulate or persist or expand in tumors in vivo, which is a favorable feature for cellular therapies. These include the following mutation constructs: CARD11-PIK3R3, MYCN_P44L, CCND3_P284S, GATA3_Y63X, STAT3_G618R, TNFRSF1B_G256C, CARMIL2_Q575E, RHOA_C16R, JAK1_G1097A, PLCG1_S520F, STAT3_N647I, JAK3_A573V, NPM-TYK, ITK-SYK, LATS1_P165T, NFKB2_K656X, CD3E_S41C, STAT3_D661I, ITK-FER, PRKCB1_D427N, VAV1_R798Q, KCNQ1_R583C, CARD11_Y361C, ECSIT_V140A, EIFS1_R89I, PDCD1_R231X, TP53_R273P, NFKB1_H67Y, TNFRSF1B_T377I, CARD11_S615F, CARD11_D357N, CARD11_E634K:S655C, CSNK1A1_S27C, CD28_T195P, RASGRP1_M261I.

Mutation constructs altered in vivo persistence in ways unattainable by wild-type gene overexpression (Table 3). For example, while wild-type CARD11 showed a negative log 2 fold change, the CARD11-PIK3R3 fusion and 4 CARD11 point mutations (CARD11 p.Y361C, p.S615F, p.D357N, and p. E634K:S655C) all showed positive log 2 fold change. Similarly, TNFRSF1B p.G256C and p.T377I mutation showed positive log 2 fold change while TNFRSF1B wild-type showed a negative log 2 fold change in tumors in vivo. Therefore, mutations can allow for improvement of in vivo persistence in tumors beyond the levels allowed by wild-type gene expression.

In vitro screening results showed correlation with in vivo screening results. Consistent with a role for PD-1 levels in regulating the in vivo persistence of CAR cells, staining for PD-1 in CD19-BBz CAR Jurkat cells showed that constructs which significantly upregulated PD-1 in response to antigen had worse persistence (FIG. 5B).

### Example 4

### Analysis of the Various Domains of the CARD11-PIK3R3 Gene Fusion

This experiment was done to elucidate which domains of the CARD11-PIK3R3 gene fusion were responsible for the gain of function of CARD11-PIK3R3.

The CARD11-PIK3R3 fusion was initially identified in a patient with CD4+ leukemic cutaneous T cell lymphoma (L. Wang et al., Genomic profiling of Sézary syndrome identifies alterations of key T cell signaling and differentiation genes. Nature Genetics 47, 1426-1434 (2015)).

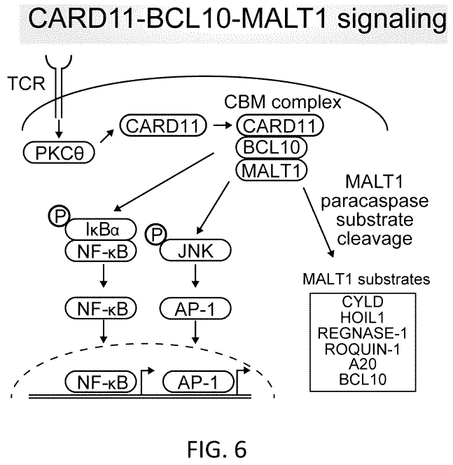

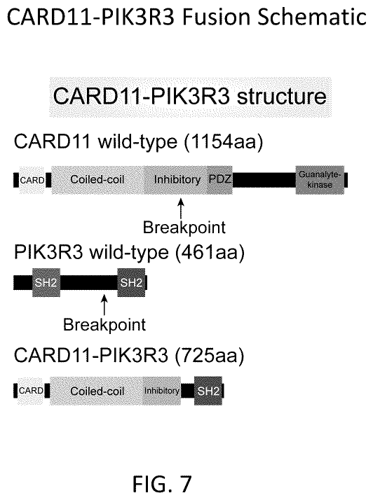

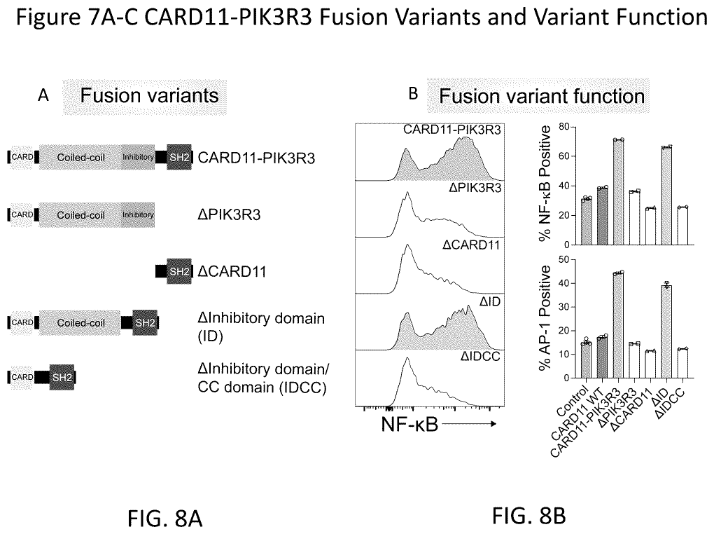

The product of the translocation between CARD11 and PIK3R3, (SEQ ID NO: 160) results in a gene fusion of the N-terminal CARD11 protein CARD domain, coiled-coil domain, and part of the inhibitory domain with an SH2 domain from the C-terminus of PIK3R3 (FIG. 7). Because of the important role of CARD11 in mediating antigen dependent signaling in T cells (FIG. 6) and the partial lack of inhibitory domain in the fusion protein, the CARD11-PIK3R3 fusion was tested to determine if it alters CBM complex signaling.

The inventor/disclosers generated variants lacking specific domains and tested their ability to induce NF-κB and AP-1 signaling (FIG. 8A-8B). Deletion of the PIK3R3 component of the fusion abolished signaling activity, but the PIK3R3 component was not sufficient on its own to induce these pathways. Therefore, the CARD11-PIK3R3 fusion does not gain function simply by truncating part of the CARD11 protein, but rather requires the C-terminal PIK3R3 to activate downstream signaling cascades. The remaining part of the inhibitory domain was not required for fusion gain-of-function, however removing the coiled-coil domain or the entire CARD11 portion of the fusion component prevented function.

### Example 5

### Truncations of the CARD11-PIK3R3 Gene Fusion

This experiment was done to characterize the structure-function relationship of the CARD11-PIK3R3 gene fusion (SEQ ID NO: 206).

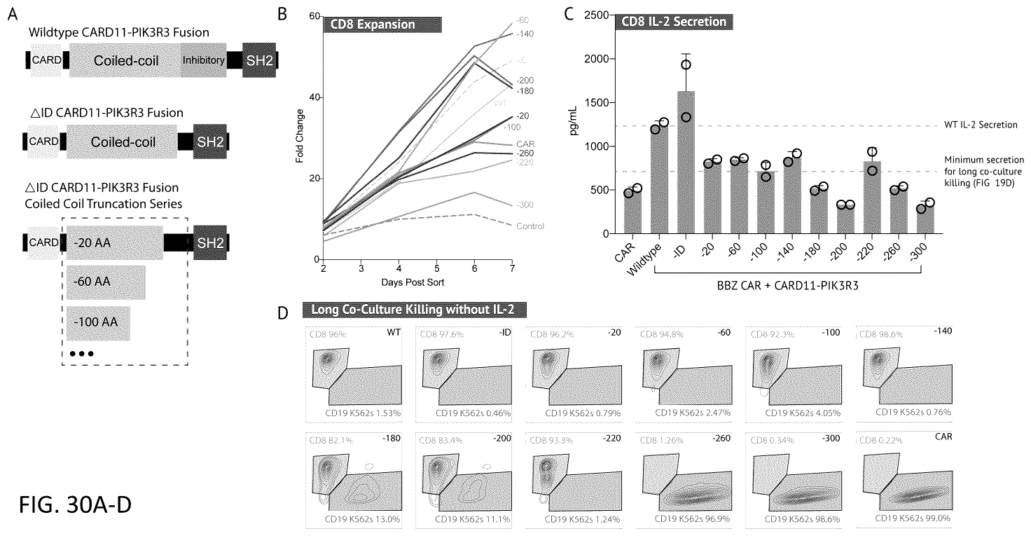

The inventors/disclosers introduced various truncations of the fusion polypeptide (SEQ ID NO: 226, 228, 230, 232, 234, 236, 238, 240, 242, 244, 246, 248, 250, 252, 254, 256) as shown in FIG. 30A. SEQ ID NOS: 226, 228, 230, 232, 234, 236, 238, 240, 242, 244, 246, 248, 250, 252, 254, 256 contain the sequence of SEQ ID NO: 206 with a 20, 40, 60, 80, 100, 120, 140, 160, 180, 200, 220, 240, 260, 280 or 300 amino acid deletion respectively. The various truncations were assessed in CD8+CD19-BBz CAR T cells over a period of 7 days after removal from anti-CD3/anti-CD28 bead stimulation (FIG. 30B). IL-2 secretions were analyzed by ELISA after co-culturing CD19-BBz CAR CD8+ T cells with CARD11-PIK3R3 truncations at 1:1 ratio with CD19 target cells for 24 hours. CD8+ and CD19K562 populations after 14-day co-culture together without supplemental IL-2 were assessed and the results are shown in FIG. 30D. The long co-culture killing assay (FIG. 30D) demonstrated that some truncated versions of the fusion (−ID, −20, −60, −100, −140, −220) induced persistent target cell killing without IL-2 supplementation similar to that of the WT (full length) version of the fusion. In contrast, some truncated versions of the fusion (−180, −200, −260, −300) were not able to induce persistent target cell killing without IL-2 supplementation and looked more similar to the CAR alone condition. These results indicate that the coiled-coil domain of CARD11 could be truncated without losing the established phenotype of the full-length fusion, though certain truncations lead to loss of function. Additionally, the IL-2 secretion data (FIG. 30C) indicates that only the −ID fusion variant produces similar amounts of IL-2 as compared to the WT (full length) version of the fusion (dotted line labeled WT IL-2 secretion). However, when comparing the IL-2 secretion data to the long co-culture killing data, the inventors/disclosers hypothesize that there is a minimum threshold of IL-2 secretion required to observe the long co-culture killing without IL-2 phenotype. In FIG. 30C this minimum threshold is indicated with a labeled dotted line. The fusion variants that were unable to killing CD19-K562 targets in the long co-culture killing without IL-2 assay (FIG. 30D) are the variants that fall below the hypothesized IL-2 secretion minimum threshold in FIG. 30C.

### Example 6

### CARD11-PIK3R3 Promotes Assembly and Signaling of the CBM Complex

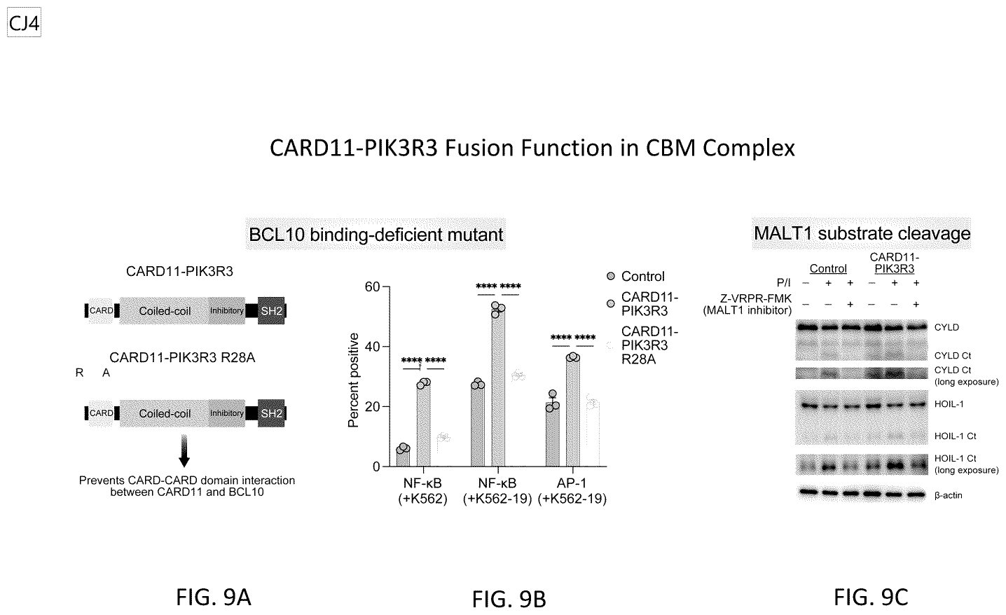

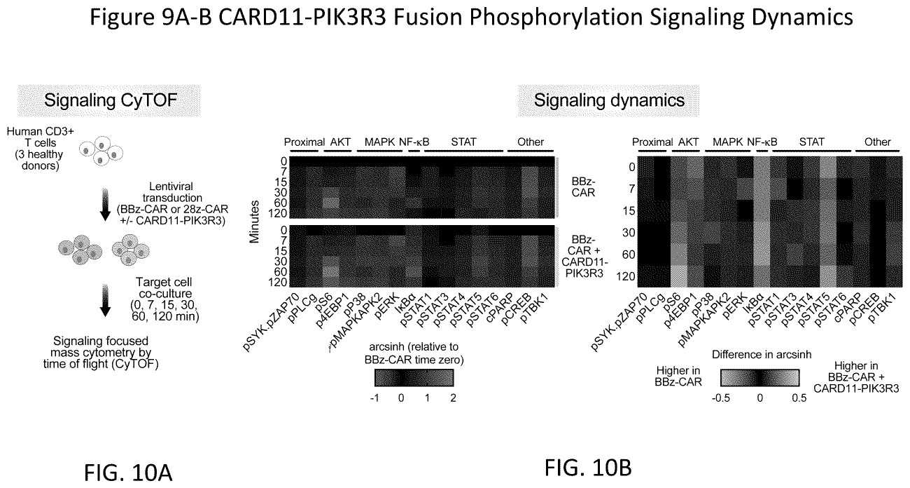

To test whether CARD11-PIK3R3 promotes assembly and signaling of the CBM complex through binding to BCL10, a mutant version of CARD11-PIK3R3 was generated with an amino acid substitution (R28A) at the BCL10 binding interface of the CARD domain of CARD11, previously reported to be essential for CARD11-BCL10 binding. It was observed that BCL10 binding was important for both tonic NF-κB and CAR signaling induced NF-κB and AP-1 (FIGS. 9A and 9B). In addition, western blotting of canonical MALT1 substrates CYLD and HOIL-1 indicated increased tonic and stimulation-induced cleavage of these proteins in the presence of the fusion (FIG. 9C). MALT1 inhibitor treatment demonstrated cleavage of these proteins was indeed MALT1 dependent (FIG. 9C). Altogether, these data indicated that the CARD11-PIK3R3 fusion protein enhances CBM complex signaling.

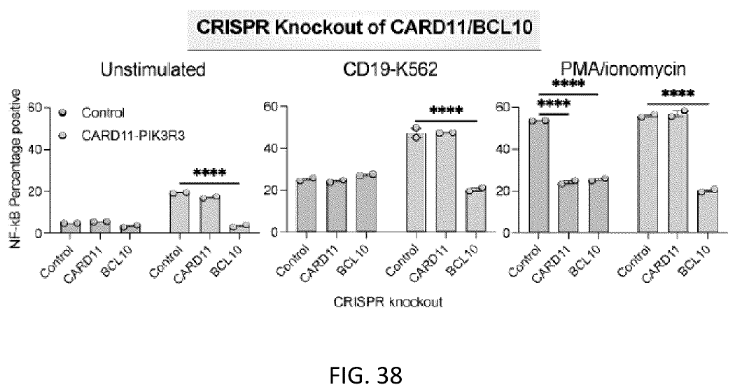

To test whether CARD11 or BCL10 were required for the function of CARD11-PIK3R3, CRISPR knockout experiments were performed. CRISPR/Cas9 was used to knock out CARD11 or BCL10 in the BBz-CAR Jurkat signaling cell line. In unstimulated, CAR-stimulated, and pharmacological TCR stimulated conditions, a reliance of CARD11-PIK3R3 on BCL10 but not wild-type CARD11 was observed (FIG. 38). In contrast, control cells relied on both CARD11 and BCL10 for TCR-triggered activation of NF-κB as expected. Interestingly, cells lacking CARD11-PIK3R3 did not depend on either CARD11 or BCL10 for CAR-dependent signaling, indicating that CBM may not be optimally engaged during CAR-T cell signaling (FIG. 38).

### Example 7

### Effects of CARD11-PIK3R3 Expression on Signaling in Primary Human T Cells

This example was to examine CARD11-PIK3R3 fusion expression in primary human T cells.

Human CD3+ T cells from three healthy donors were lentivirally transduced with CD19-CD28z or BBz CARs, with or without the CARD11-PIK3R3 fusion. CAR T cells were analyzed by CyTOF at 0, 7, 15, 30, 60, and 120 minute time points of co-culture with CD19 expressing cells (FIG. 10A). Consistent with activation of NF-κB signaling induced in Jurkat cells, both CD19-CD28z and CD19-BBz CAR cells expressing the CARD11-PIK3R3 fusion displayed significantly reduced levels of IκBα, a negative regulator of NF-κB which is degraded downstream of CBM complex activation (FIG. 10B). Furthermore, CD19-BBz CAR cells showed enhanced signaling through two pathways downstream of the CBM complex, PI3K/Akt (p4EBP1) and MAPK, though these were not significantly different in CD19-CD28z CAR cells. Consistent with signaling differences being mediated through the fusion itself and not differences in transduction from the CAR, no differences were observed in proximal signaling events.

This data indicated that CARD11-PIK3R3 expression altered downstream signaling events in primary cells following CAR engagement.

### Example 8

### CARD11-PIK3R3 Enhances Gene Expression

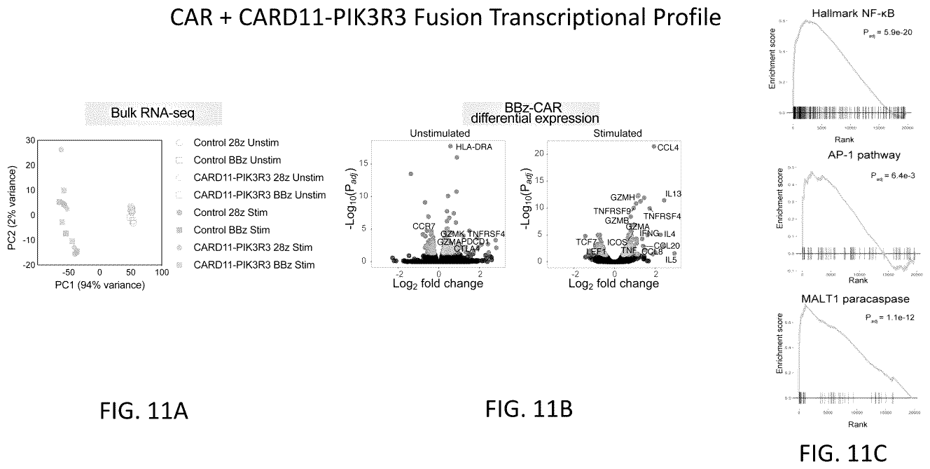

To examine potential difference in gene expression, bulk RNA-sequencing of human CD8+ T cells from three healthy donors was performed with and without CAR stimulation through co-culture with CD19 expressing targets. Principal component analysis revealed strong transcriptional differences due to stimulation, but minimal global differences in the transcriptomes of CARD 11-PIK3R3 cells from controls (FIG. 11A). Differential expression analysis revealed that genes significantly upregulated in CARD11-PIK3R3 CD19-BBz CAR cells compared to controls in the stimulated state were genes normally induced in control cells by CAR signaling, that were enhanced in the presence of the fusion (FIG. 11B). This suggests that rather than inducing an entirely distinct transcriptional state, CARD11-PIK3R3 expression augments the expression of a subset of CAR induced genes. These include activation markers (IL2RA, CD69), cytotoxic and effector molecules (IFNG, TNF, IL4, IL5, IL13, GZMA, GZMB), chemokines (CCL4, CCL20, CCL8), and co-stimulatory molecules (ICOS, OX40, 4-1BB, GITR). Consistent with the inventors/disclosers' biochemical analyses, gene set enrichment utilizing a previously reported set of genes which are downregulated in response to MALT1 paracaspase inhibition in human T cells revealed significant enrichment of these genes in CARD11-PIK3R3 expressing cells, as well as NF-κB and AP-1 induced genes (FIG. 11C).

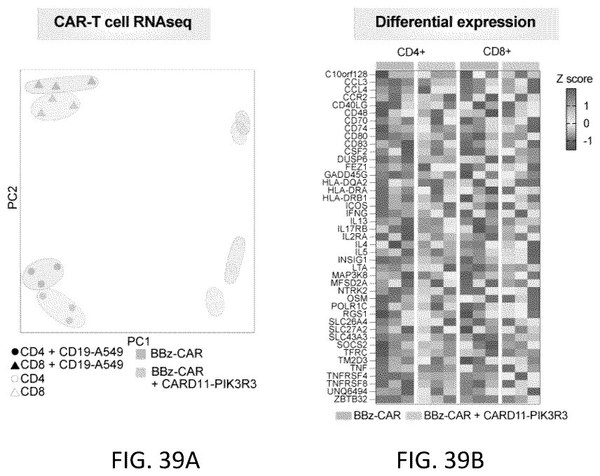

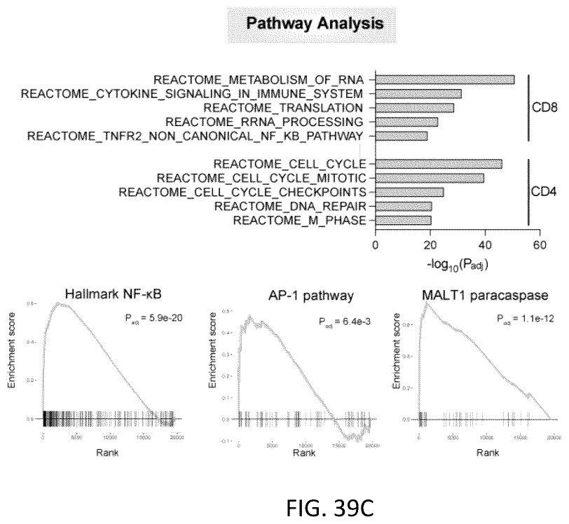

RNA-sequencing of human CD4+ and CD8+ T cells from three healthy donors, with and without antigen stimulation (Supplemental Table 2). Principal component analysis demonstrated the most dramatic transcriptional differences were induced by CAR-dependent stimulation, suggesting that CARD11-PIK3R3 expression is not sufficient to cause primary cells to adopt a fully antigen activated phenotype (FIG. 39A). By comparing transcripts upregulated in both CD4+ and CD8+ T cells upon CAR stimulation, we identified a core group of 43 genes modulated by CARD11-PIK3R3 (FIG. 39B) These included several transcripts with important roles in CAR T cell function and effector cytokine production (FIG. 39B), including activation markers (IL2RA), cytotoxic and effector molecules (IFNG, TNF, IL4, IL5, IL13), chemokines (CCL4), and co-stimulatory molecules (ICOS, TNFRSF4[OX40]). Several of these genes, such as ICOS48 and OX4049, have been previously suggested to favorably impact anti-tumor responses. Interestingly, CARD11-PIK3R3 expressing CD4+ T cells were enriched for gene signatures related to cell cycle as compared to CD8+ T cells, which were enriched most for RNA metabolism, cytokine signaling, and translation signatures (FIG. 39C). Consistent with our biochemical analyses, gene set enrichment identified enrichment of NF-κB, AP-1, and MALT1 paracaspase signatures (FIG. 39C).

### Example 9

### CARD11-PIK3R3 Provides Proliferative Advantage to CD3 T Cells

The following experiment shows how increased CBM complex signaling affects the activation state and effector phenotypes in primary CAR T cells.

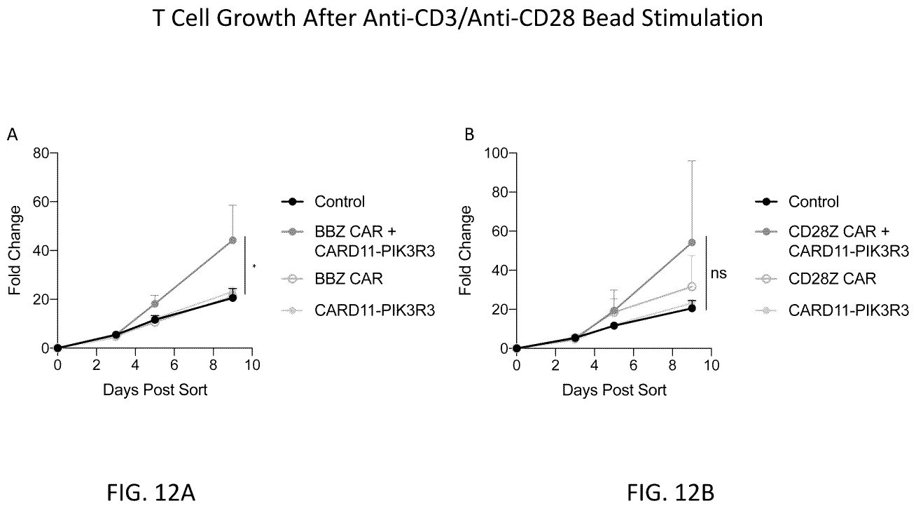

Primary T cells were co-transduced with a CD19-BBz or CD19-CD28z CAR and the CARD11-PIK3R3 fusion protein. After removing anti-CD3/CD28 beads and sorting for a purified population, cells were expanded and rested in culture for approximately one week. In transduced CD3 T cells, greater expansion of CD19-BBZ CAR with CARD11-PIK3R3 T cells was observed as compared to the CD19-BBz CAR T cells alone (FIG. 12A). However, this observation was not replicated in the CD19-CD2z T cell only population (FIG. 12B), suggesting the fusion provides a proliferative advantage particularly when paired with a BBz CAR.

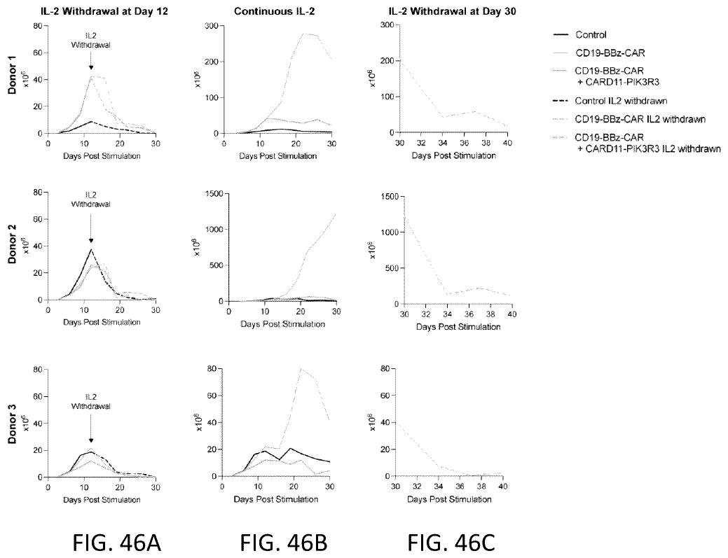

Further experiments were performed to determine if CARD11-PIK3R3 could induce cytokine independent growth in primary CAR T cells. To do so, we co-transduced primary T cells with a CD19-BBz-CAR and CARD11-PIK3R3. After removing anti-CD3/CD28 stimulation and sorting for a purified population, cells were expanded and rested in culture with IL-2. CARD11-PIK3R3 improved the expansion of CAR-T cells in the presence of IL-2, however removal of IL-2 either early or late in the culture led to rapid T cell population contraction (FIGS. 46A-46C).

### Example 10

### Antigen-Induced Activation States

To assess antigen-induced activation states, CD8+ T cells were co-cultured with K562-CD19 targets for 24 hours, and then assessed the expression of activation markers via flow.

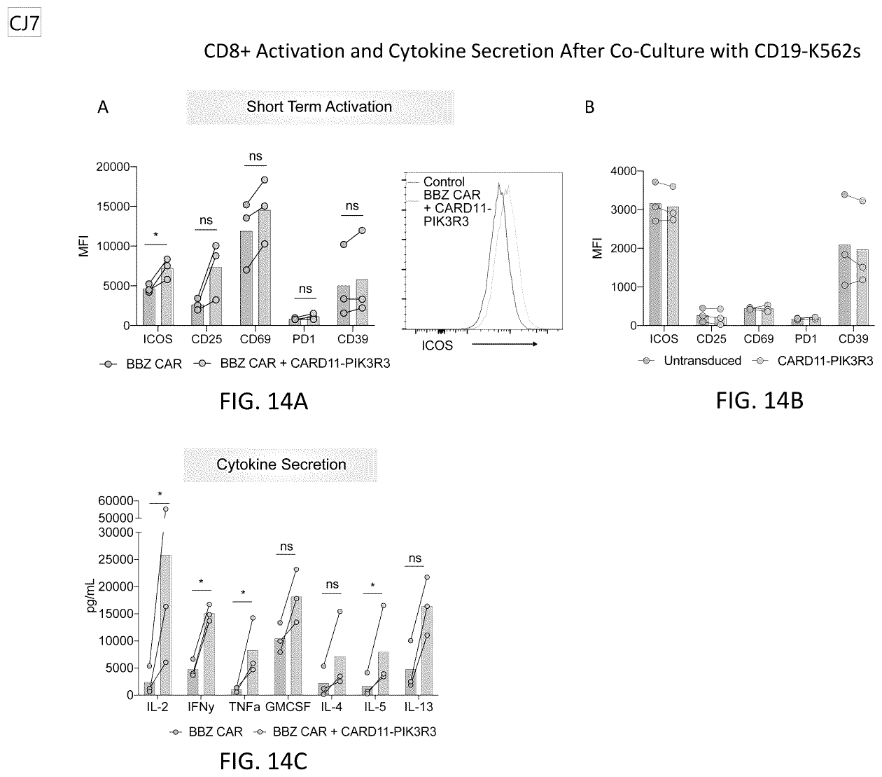

Most short term activation markers (CD25, CD69, PD-1, CD39) by both CD19-BBz CAR T cells and CD19-BBz CAR with CARD11-PIK3R3 fusion protein T cells were upregulated (FIG. 14A). Consistent with the bulk RNAseq data, ICOS was found to be significantly upregulated in CD19-BBz CAR with CARD11-PIK3R3 fusion T cells as compared to CAR T cells alone. These observations were similar in the CD19-CD28z CAR setting. Interestingly, while Jurkat signaling data suggested that the CARD11-PIK3R3 fusion protein leads to higher basal expression of NF-κB, the inventors/disclosers did not find that CD8+ T cells expressing only the CARD11-PIK3R3 fusion had significantly higher expression of activation markers as compared to control (untransduced) T cells (FIG. 14B). This suggested that while the fusion does induce some basal NF-κB signaling, it is not significant enough to cause antigen independent activation of the cell.

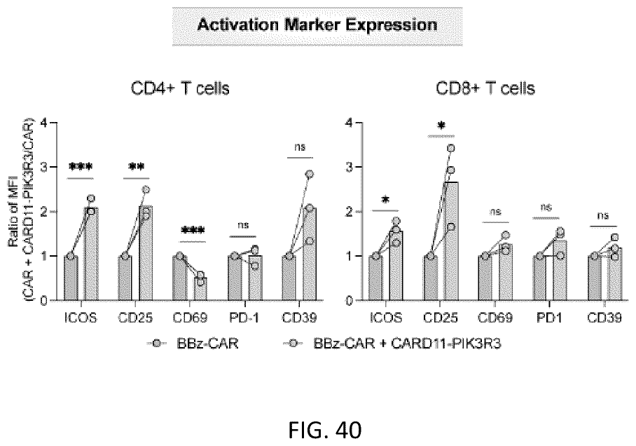

Further experiments were performed using both CD8+ and CD4+ T cells. Equivalent expression of some activation markers (PD-1, CD39) in CD19-BBz-CAR T cells with and without CARD11-PIK3R3 was observed (FIG. 40). Consistent with the bulk RNAseq data, CD25 (IL2RA) and ICOS were found to be significantly upregulated in CARD11-PIK3R3 CD19-BBz CAR T cells as compared to CAR T cells alone (FIG. 40).

### Example 11

### CARD11-PIK3R3 Upregulated Cytokines

This Example shows how CARD11-PIK3R3 upregulates various cytokines.

Bulk RNAseq analysis of activated CD19-BBz CAR with CARD11-PIK3R3 fusion T cells identified numerous upregulated cytokines (TNFα, IFN-γ, IL-5, and IL-13).

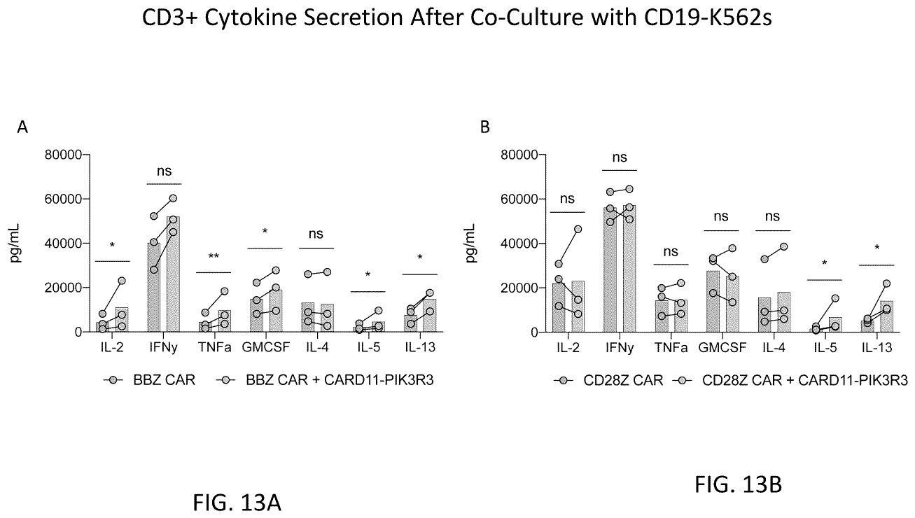

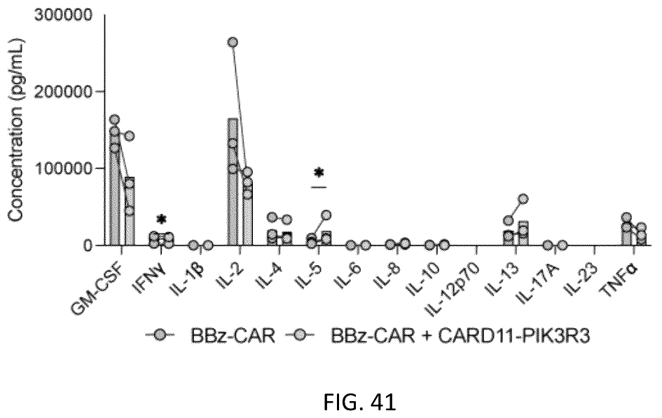

To confirm these findings at the protein level the inventors/disclosers co-cultured transduced CD8+ T cells with CD19-K562 targets for 48 hours and assessed the supernatant for a variety of cytokines. Verifying the Jurkat ELISA results, they found that the CARD11-PIK3R3 fusion induced higher secretion of IL-2, in addition to other inflammatory cytokines such as IFN-γ, TNFα and GMCSF (though not significantly increased) (FIG. 14C). Surprisingly, while cytokine secretion was substantially increased in CD8+CARD11-PIK3R CAR T cells, this was less so in CD4+CARD11-PIK3R CAR T cells, despite being originally observed in a CD4+ T cell cancer (FIG. 41) Interestingly, Th2 cytokines IL-5 was also significantly increased by expression of the fusion, indicating that the fusion induces a unique cytokine profile. Similar trends were observed in CD8+ T cells with the CD19-CD28z CAR and bulk CD3 T cells with either CD19-BBz or CD19-CD28z CAR, however in some cases the increase observed in fusion transduced cells was not significant (FIG. 13A-B).

### Example 12

### CARD11-PIK3R3, Cytotoxicity, and Growth

To understand how the expression of the CARD11-PIK3R3 fusion shaped CAR T cell cytotoxicity and growth, the following experiment was conducted.

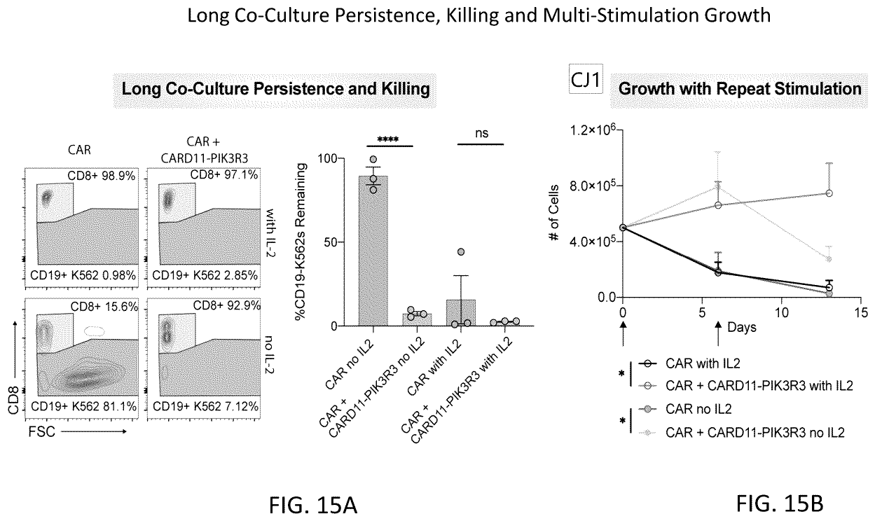

As tumor microenvironments often lack pro-survival signaling required by cytotoxic T cells, such as IL-2, the inventors/disclosers did experiments to reveal how the CARD11-PIK3R3 fusion would affect engineered T cell killing in a state of IL-2 starvation. A long-term assay was set up, where transduced T cells were mixed with CD19-K562s at a 1:1 ratio and maintained in culture for two weeks with or without supplemental IL-2. Interestingly, after two weeks of culture both the CD19-BBz CAR alone and CAR with CARD11-PIK3R3 were able to efficiently clear the CD19-K562 targets, however when cultured without supplemental IL-2, only the CD19-BBz CAR with CARD11-PIK3R3 was able to efficiently clear the CD19-K562 targets (FIG. 15A). Less dramatic differences in killing were observed for the CD19-CD28z CAR in the long co-culture killing assay.

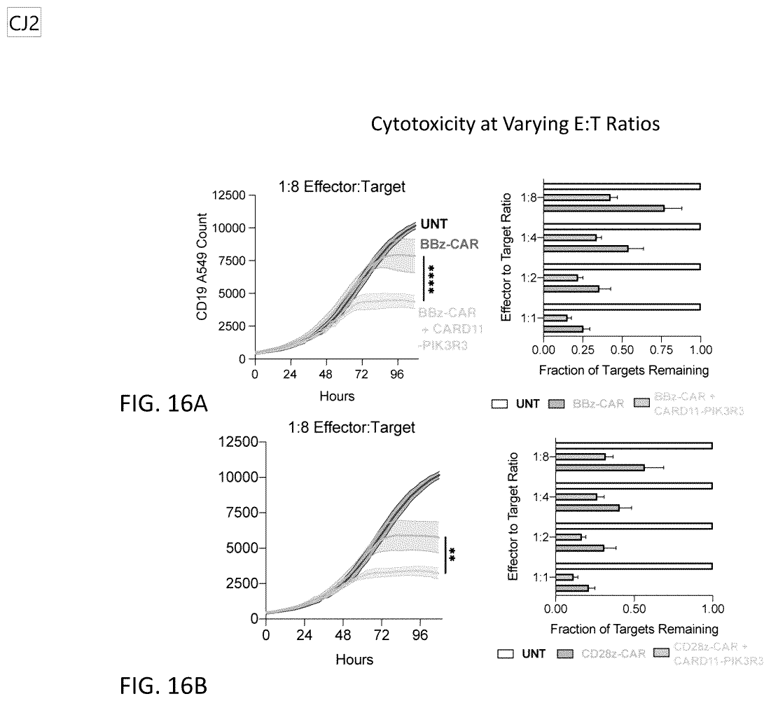

When stimulated twice in a two-week period, the inventors/disclosers noticed that regardless of supplemental IL-2, the CD19-BBz CAR alone decreased in numbers, while the CD19-BBz CAR with CARD11-PIK3R3 efficiently expanded (with IL-2), or maintained a higher number of cells (without IL-2) over time (FIG. 15B). Finally, the inventors/disclosers assessed target killing at varying ratios over time and found that at every ratio tested, the CD19-BBz CAR with CARD11-PIK3R3 better controlled target cell growth as compared to the CD19-BBz CAR alone (FIG. 16). Similar trends were noted for CD19-28z CAR with and without the CARD11-PIK3R3 fusion.

### Example 13

### CARD11-PIK3R3 Improves Fitness, Function and Efficacy of Therapeutic T cells

This Example showed how the CARD11-PIK3R3 fusion improved the fitness, function, and efficacy of therapeutic T cells in a fully immunocompetent, syngeneic setting.

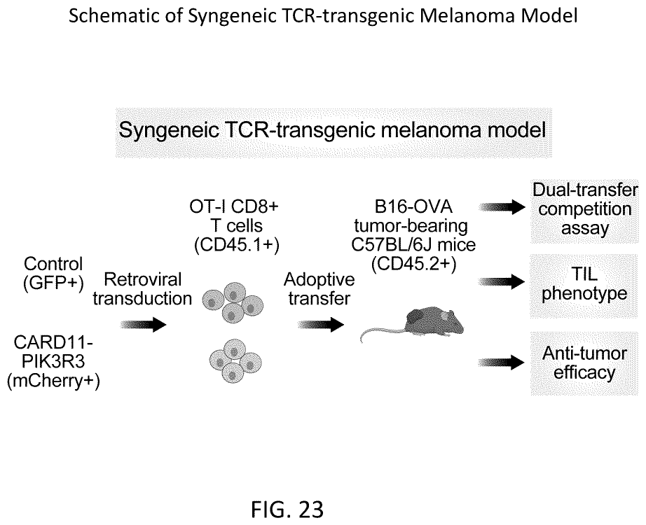

Transgenic TCR-expressing murine OT-I CD8+ T cells, which are specific for chicken ovalbumin (OVA), were collected from CD45.1 C57BL/6-Tg(TcraTcrb)1100Mjb/J (OT-I) mice. These cells were used in B16-OVA mouse model, in which B16 melanoma cells express the OVA antigen (FIG. 23).

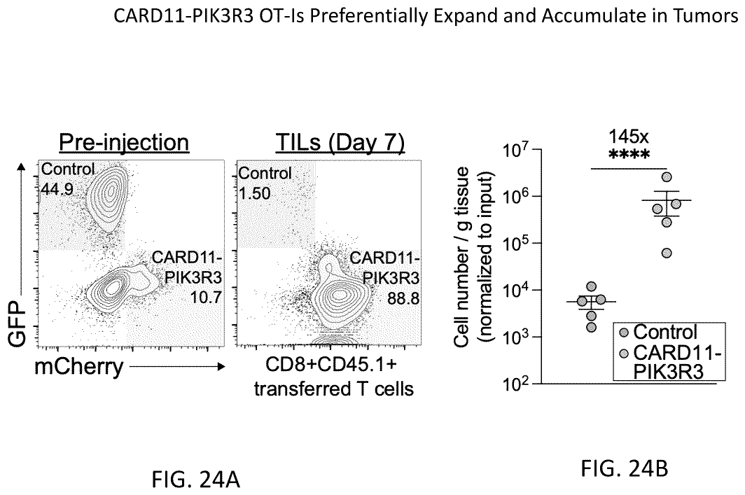

CD45.1+OT-I CD8+ T cells were transduced with either control (GFP+) or CARD11-PIK3R3 (mCherry+) retroviruses, enabling tracking of adoptively transferred cells in CD45.2+C57BL/6J hosts bearing B16-OVA melanoma tumors. To analyze in vivo accumulation, a dual-transfer system was used in which competition between transferred T cells in the same tumor can be determined. The inventors/disclosers generated a mixture of CD45.1+OT-I T cells in which approximately 10% of the cells were mCherry positive (corresponding to CARD11-PIK3R3 expressing cells) to directly compare in vivo to GFP+ control cells. Strikingly, a ˜145 fold increase in cell number (normalized to input) of CARD11-PIK3R3 expressing cells was observed compared to control cells among the TILs 7 days following transfer (FIGS. 24A and B).

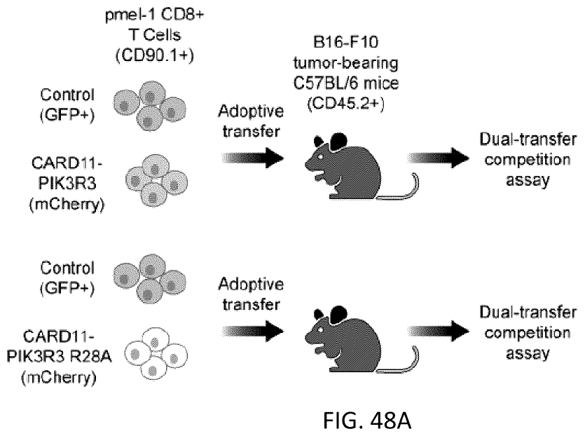

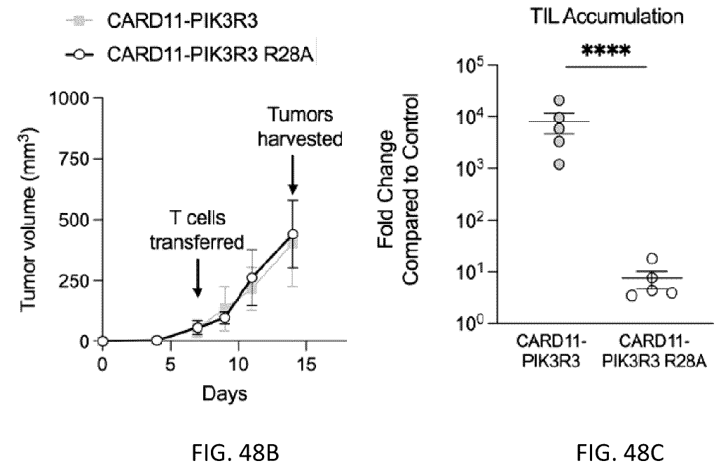

We confirmed enhanced competitive accumulation of CARD11-PIK3R3 in a second transgenic TCR mouse model, pmel-1 T cells (which recognize gp100, an endogenous melanoma antigen) against B16-F10 tumors (FIGS. 48A-48C). We performed this assay with both the wildtype CARD11-PIK3R3 and the R28A mutant to assess the dependence of the in vivo phenotypes on BCL10 interactions. Mutating the R28A binding site reduced accumulation in vivo by 1257-fold (FIGS. 48A-48C).

This corresponded to a significantly increased fraction of transferred cells among total CD8+ T cells, even without normalization to input numbers. Therefore, within the same tumor microenvironment, CARD11-PIK3R3 expressing T cells have dramatically improved accumulation compared to control cells.

### Example 14

### Characterization of the Phenotype of Cells Following Adoptive Transfer of CARD11-PIK3R3

The following test was used to characterize the phenotype of cells following adoptive transfer of either CARD11-PIK3R3 or control transduced OT-I cells.

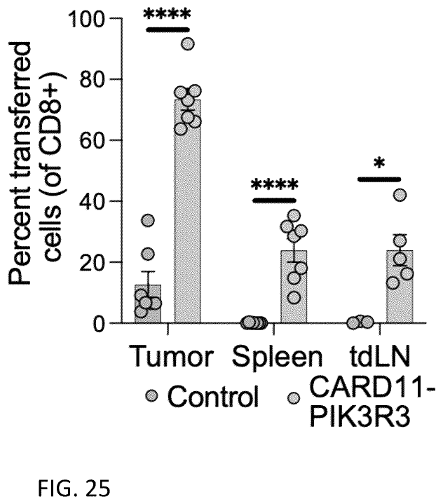

In accordance with the data obtained from the competitive dual-transfer assay, there was also enhanced accumulation of CARD11-PIK3R3 expressing cells in the tumor when transferred separately. In addition, the inventors/disclosers detected a higher proportion of CARD11-PIK3R3 OT-I cells compared to control OT-I cells in the spleen and tumor draining lymph node, though to a lesser extent than observed in the tumor. (FIG. 25)

### Example 15

### Effector Functions of CARD11-PIK3R3

The following test was used to characterize the effector functions of the CARD11-PIK3R3 fusion protein.

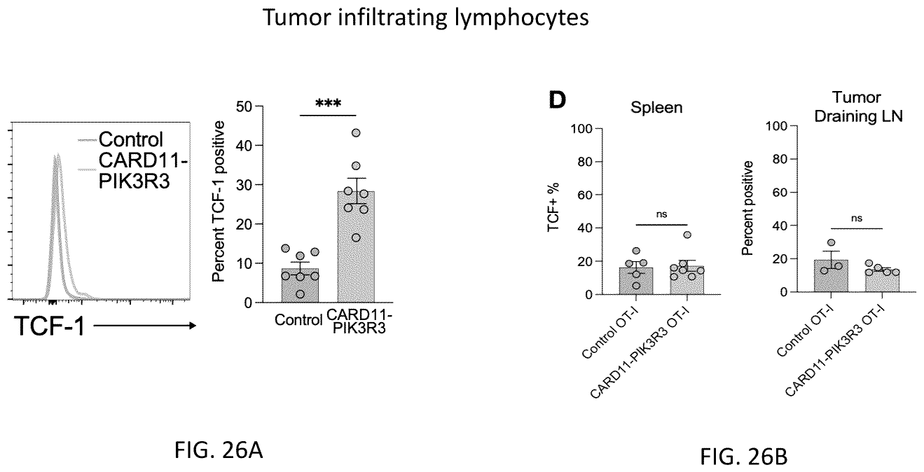

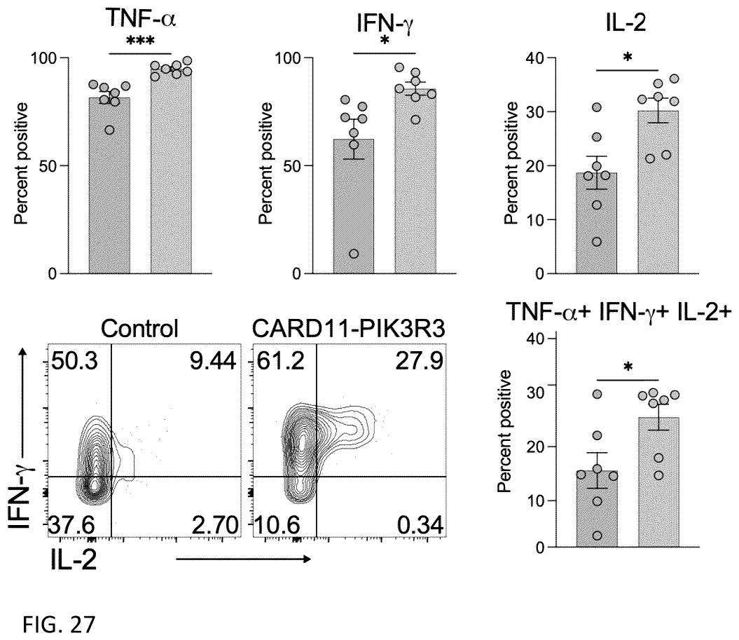

Ex vivo restimulation and intracellular cytokine staining of CARD11-PIK3R3 OT-I cells revealed enhanced effector functions of fusion expressing cells, including higher production of TNF-α, IFN-γ, and IL-2 (FIG. 27). Furthermore, the proportion of polyfunctional cells producing all three of these effector cytokines was significantly elevated in CARD11-PIK3R3 OT-I cells. Additionally, CARD11-PIK3R3 expression substantially increased the expression of the stemness-associated transcription factor TCF1 in TILs (FIG. 26A). Consistent with tumor-specific reprogramming, TCF1 expression was not significantly different in either tumor draining lymph nodes or spleen. (FIG. 26B)

These results indicate that CARD11-PIK3R3 promotes the intra-tumoral accumulation of highly functional, stem-like T cells.

### Example 16

### Anti-Tumor Efficacy of CARD11-PIK3R3 in a Syngeneic, TCR Transgenic Melanoma Mouse Model

The following experiment was done to determine the effects of CARD11-PIK3R3 on the therapeutic efficacy of T cells in a syngeneic, immunocompetent systems.

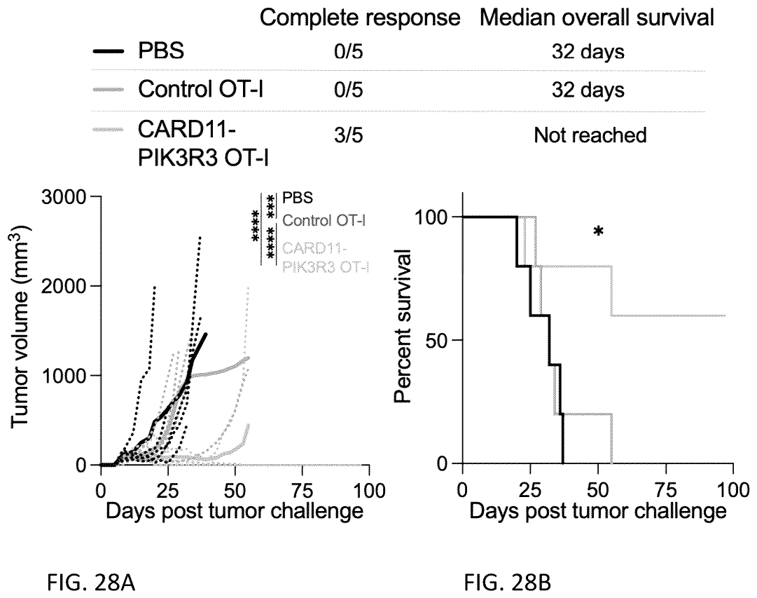

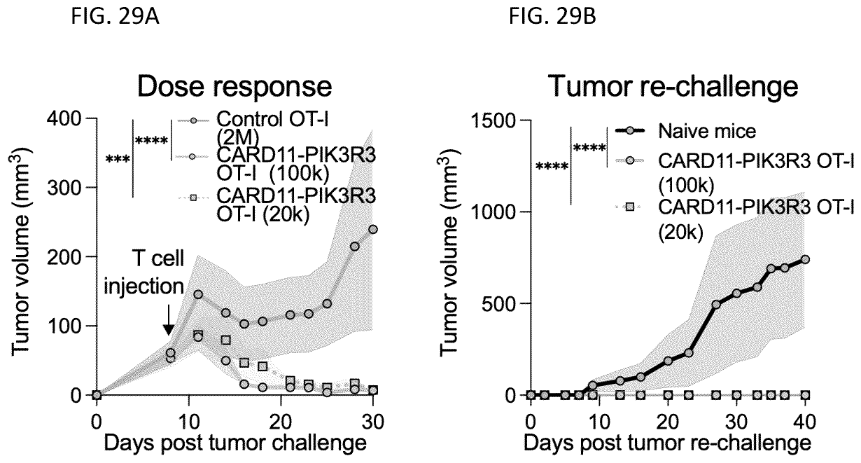

On day 12 following inoculation with B16-OVA tumors subcutaneously, mice were treated with PBS control or 2×10⁶ OT-I cells transduced with either control or CARD11-PIK3R3 retrovirus, without pre-conditioning or lymphodepletion. CARD11-PIK3R3 cells mediated significantly enhanced control of tumor volume (FIG. 28A). In addition, CARD11-PIK3R3 OT-I cells promoted prolonged overall survival (FIG. 28B). 60% of mice (⅗) receiving the CARD11-PIK3R3 OT-Is achieved complete clearance of tumors at 85 days following tumor challenge, at which point no PBS or control OT-I treated mice survived. Altogether, these data indicate that CARD11-PIK3R3 expressing T cells have superior therapeutic function in vivo. Reduced cell numbers are routinely used as “stress tests” to quantify relative efficacy of modified T cell therapies. Moreover, these data suggest that the CARD11-PIK3R3 fusion may overcome one key limitation of cell therapies, i.e. the ability to manufacture sufficient cells for therapeutic efficacy in humans. To test this, therapeutic efficacy of 20,000 and 100,000 CARD11-PIK3R3 transduced OT-I cells against 2 million control OT-I cells in B16-OVA tumor bearing mice was compared. Strikingly, CARD11-PIK3R3 enabled tumor control with both 20-fold and 100-fold lower dose than control cells (FIG. 29A)

Current T cell therapies often lack long term persistence, failing to form memory populations after clearance of the primary tumor, resulting in high incidence of relapse. After more than two weeks of initial tumor clearance, CARD11-PIK3R3 OT-1 treated animals treated with low cell doses or naïve controls were rechallenged with B16-OVA tumor cells in the contralateral flank. The CARD11-PIK3R3 OT-I treated animals were protected from tumor development compared to naïve, untreated mice (FIG. 29B). The rejection of a secondary tumor challenge suggests the CARD11-PIK3R3 OT-Is develop a long term, memory phenotype that is capable of responding to and suppressing tumor reoccurrence. This phenotype is highly desirable in cancer therapies, allowing for a single engineered therapy to respond to primary tumors and to prevent relapse, inducing durable clinical remission. Altogether, these data indicate that CARD11-PIK3R3 expressing T cells have superior therapeutic function in vivo.

### Example 17

### Enhancement of Other Cell Types than the Original Cell by Mutation Constructs

The CARD11-PIK3R3 fusion was initially uncovered in a patient with CD4+ T cell lymphoma (Wang et al., Genomic profiling of Sézary syndrome identifies alterations of key T cell signaling and differentiation genes. Nature Genetics 47, 1426-1434 (2015). The OT-I data described in Example 16 is a CD8+ only cell therapy model, and the CARD11-PIK3R3 fusion dramatically enhanced the function of these CD8+ T cells in vivo, despite being a sequence derived from a CD4+ T cell. Therefore, mutations described herein can have therapeutic value in cell types that differ from those the mutations originally occur in.

### Example 18

### CARD11-PIK3R3 Improves the Anti-Tumor Efficacy of Mouse CAR T Cells in a Syngeneic Melanoma Model

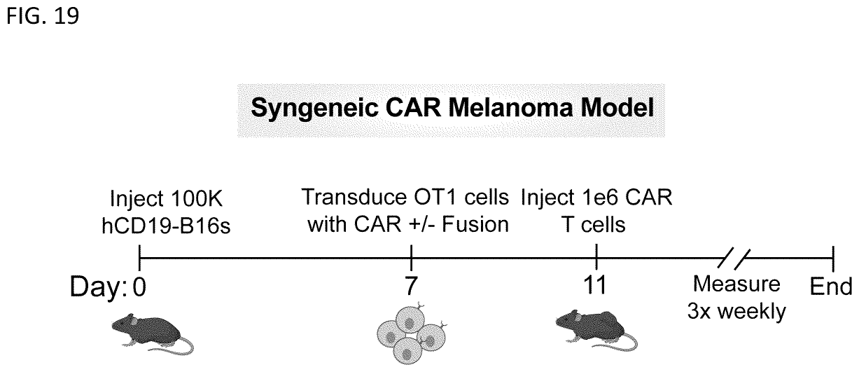

T cells were collected from C57BL/6-Tg(TcraTcrb)1100Mjb/J (OT-I) mice and transduced with a hCD19-CAR (CD19-BBz CAR), with or without the CARD11-PIK3R3 fusion (FIG. 19). T cells were transferred to mice bearing hCD19-B16 melanoma tumors. In this model B6.SJL-Ptprca Pepcb/BoyJ mice receive a subcutaneous injection of 1×10⁵ hCD19 expressing B16 tumor cells in the hind flank. Eleven days after tumor inoculation, 1×10⁶ CAR+ mouse T cells were dosed to tumor bearing animals, and tumor growth was tracked via calipers measurements taken 3× weekly. Tumor volume was calculated using the following equation: (Length×Width×Width)/2, where length is the longest measurement. Mice were euthanized when the tumor reaches 2000 mm3 or 2 cm in either length or width.

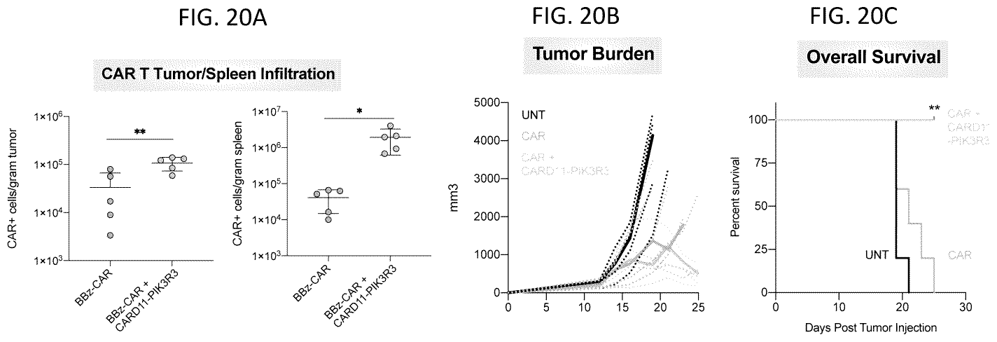

CARD11-PIK3R3 CAR T cells had significantly increased accumulation in the tumor and the spleen, significantly increased ability to control tumor volume, and significantly increased overall survival (FIG. 20). Therefore, CARD11-PIK3R3 enhances the efficacy of CAR T cells in vivo, in a syngeneic setting without lymphodepletion, radiation, or chemotherapy.

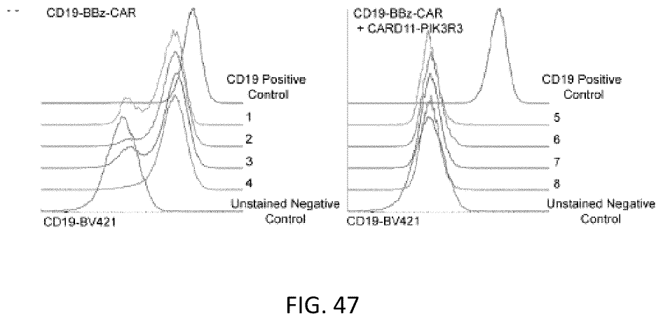

In a repeated study, CD19-BBz-CAR and CARD11-PIK3R3 CD19-BBz-CAR T cell treated tumors were assessed at tumor endpoint by flow cytometry. CD19-BBz-CAR treated tumors maintained CD19 expression, while CARD11-PIK3R3 CD19-BBz-CAR T cell treated tumors were uniformly CD19 negative, suggesting antigen loss as the mechanism of relapse (FIG. 47).

### Example 19

### CARD11-PIK3R3 Improves the Anti-Tumor Efficacy of Human CAR T Cells in a Xenograft Leukemia Model

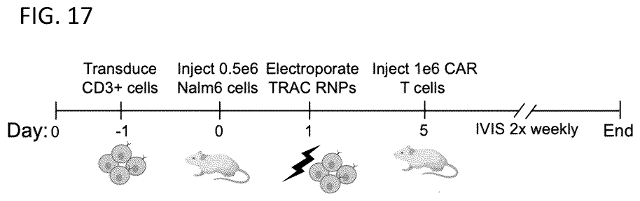

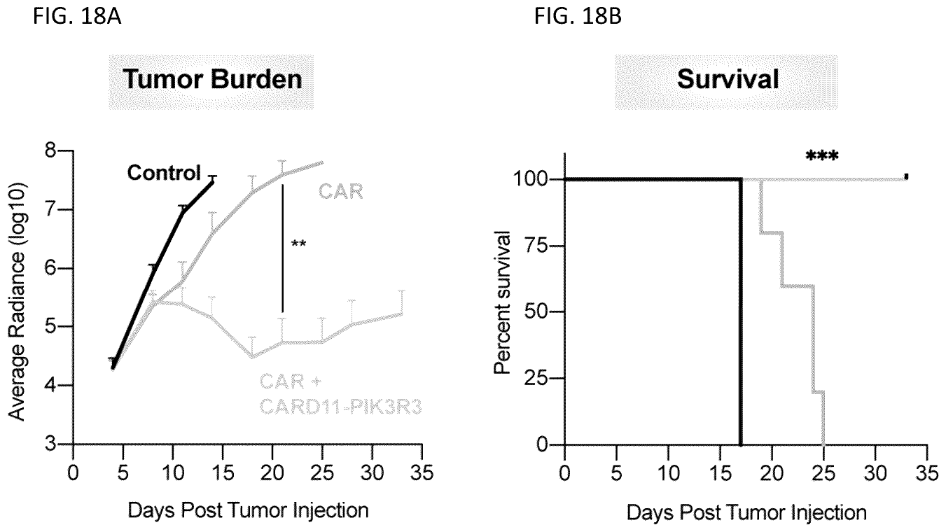

Human CD3+ T cells were transduced with a CD19-BBz CAR with or without the CARD11-PIK3R3 fusion. T cells were then electroporated with Cas9 RNPs targeting the TCR alpha locus to knockout the endogenous T cell receptor. NSG mice bearing Nalm-6-luciferase leukemia were injected with CAR T cells (FIG. 17). Mice treated with CARD11-PIK3R3 CAR T cells had significantly lower tumor burden and a significantly improved survival (FIG. 18).

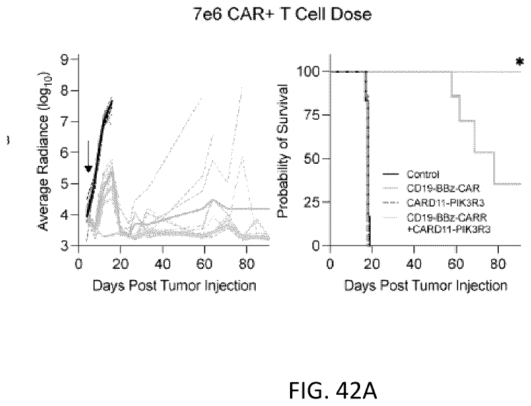

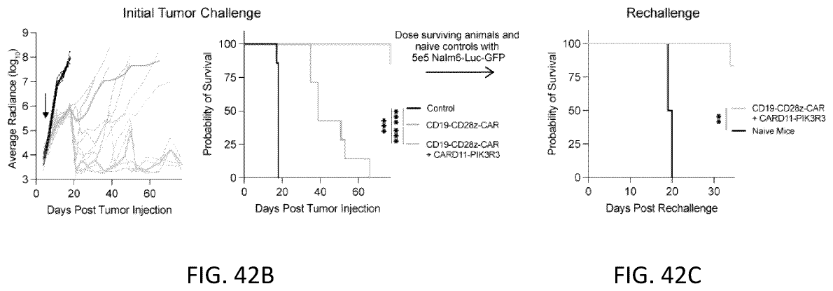

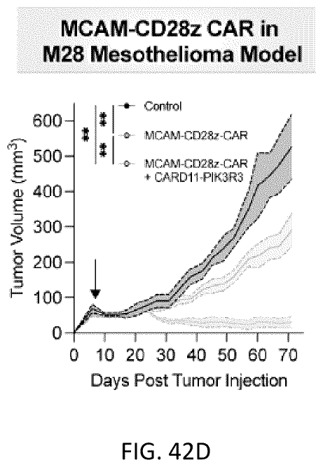

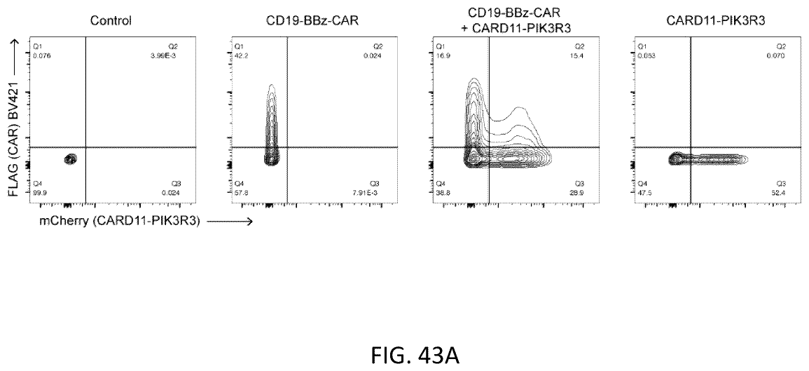

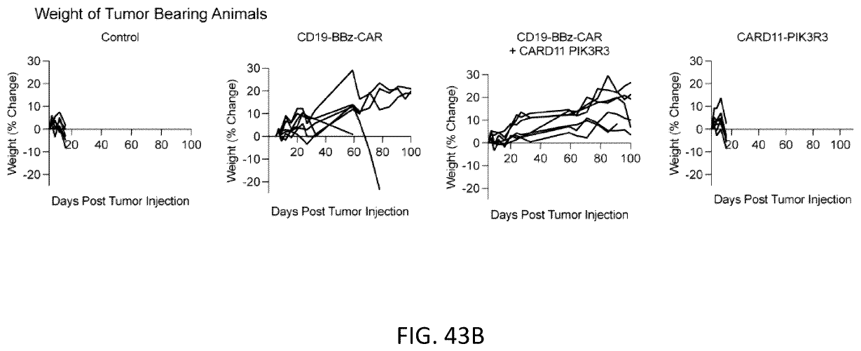

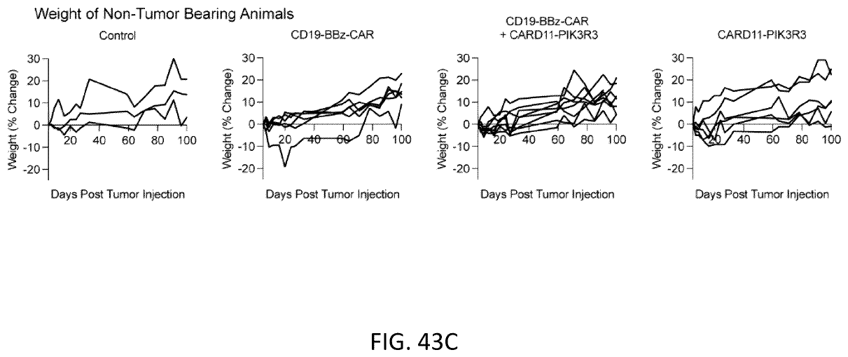

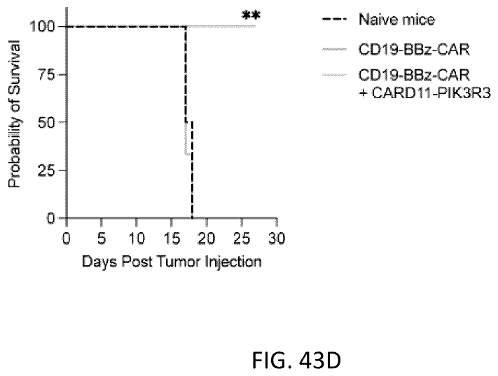

The improved efficacy of CD19-BBz-CAR T cells was also observed at higher doses of 7×10⁶ CAR+ T cells, where 4 of 7 CD19-BBz-CAR treated animals that initially controlled tumor began relapsing with NALM-6 disease, while 7 of 7 CARD11-PIK3R3 CD19-BBz-CAR T cell treated animals did not relapse (FIG. 42A, FIG. 43A). We also determined that CARD11-PIK3R3 expressing T cells alone had no anti-tumor effect, demonstrating that the anti tumor effects observed are antigen-specific, and dependent on CAR activity (FIG. 42A). To address safety concerns, we also determined that NALM-6 bearing animals that controlled tumors with CAR or CARD11-PIK3R3 CAR T cell treatment gained weight over the course of 100 days, losing weight only when NALM-6 relapse occurred (FIG. 42A, FIG. 43B). Similarly, non-tumor bearing mice tolerated high doses of control T cells, CAR T cells, CAR+CARD11-PIK3R3 T cells (FIG. 43C). Finally, upon 5×10⁵ NALM-6 tumor rechallenge (to animals that had eliminated the leukemia and had not developed symptoms of GVHD), we found that CD19-BBz-CAR treated animals succumbed to NALM-6 disease at a similar rate as naïve age matched controls, while CARD11-PIK3R3 CD19-BBz-CAR T cells prevented leukemic growth (FIG. 43D). These data indicate that CARD11-PIK3R3 enhances therapeutic efficacy while maintaining safety, even at high T cell doses, in vivo.

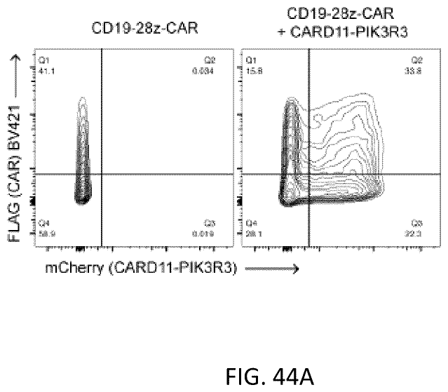

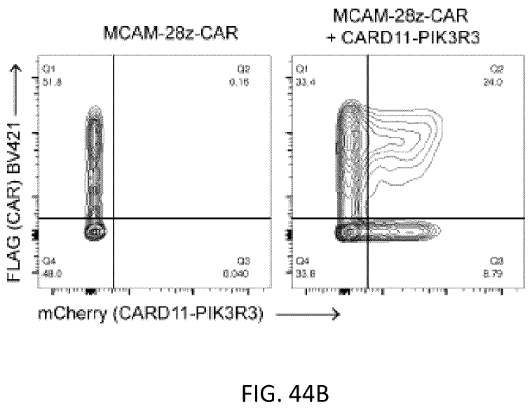

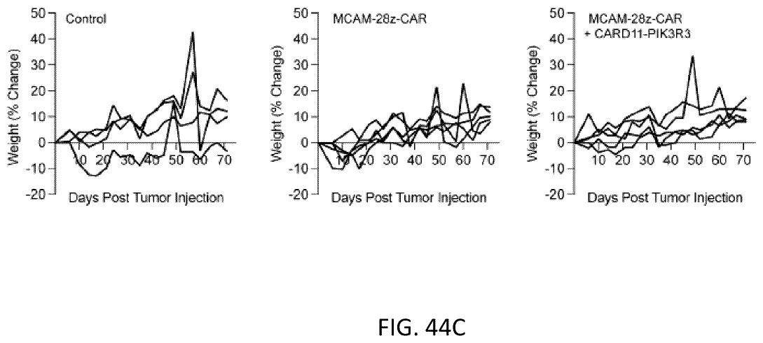

We next determined whether CARD11-PIK3R3 could improve CD19-CD28z-CAR T cell activity at low doses in the NALM6 leukemia model described above. We thus dosed NALM6-bearing mice with 4×10⁵ CD19-CD28z-CAR T cells, or CARD11-PIK3R3 CD19-CD28z-CAR T cells (FIG. 44A) and found that CAR T cell treated animals succumbed to disease, while CARD11-PIK3R3 CAR T cells cleared two successive NALM6 challenges (FIGS. 42B-42C). Therefore, the anti-tumor efficacy of CARD11-PIK3R3 expression is not limited to CARs with 4-1BB costimulatory domains, but also improves those with CD28 domains, suggesting that CARD11-PIK3R3 expression could broadly benefit CAR T cell therapies.

Finally, we sought to determine how CARD11-PIK3R3 could perform in a xenograft solid tumor model. Here, we used the subcutaneous model of mesothelioma (M28), which naturally expresses the tumor associated antigen MCAM55. We manufactured and dosed tumor bearing animals with 5×10⁵ MCAM targeted CD28z-CAR T cells with or without CARD11-PIK3R3, or control T cells (FIG. 44B). Animals maintained weight throughout the study (FIG. 44C), indicating therapy was well tolerated. While MCAM-CD28z-CAR treatment delayed tumor growth compared to the control, both control and CAR T cell treated tumors progressively increased in size (FIG. 42D). In contrast, CARD11-PIK3R3 MCAM CD28z-CAR T cell treated animals controlled M28 tumor growth over the course of approximately 70 days (FIG. 42D). Therefore, CARD11-PIK3R3 safely improves the long-term therapeutic efficacy of human CAR T cells in vivo in both hematological and solid tumor settings. Furthermore, CARD11-PIK3R3 CAR T cells protect from relapse (rechallenge) as compared to CAR T cells alone.

### Example 20

### CARD11-PIK3R3 Improves the Anti-Tumor Efficacy of Human CAR T Cells in a Human TCR-Based Xenograft Model

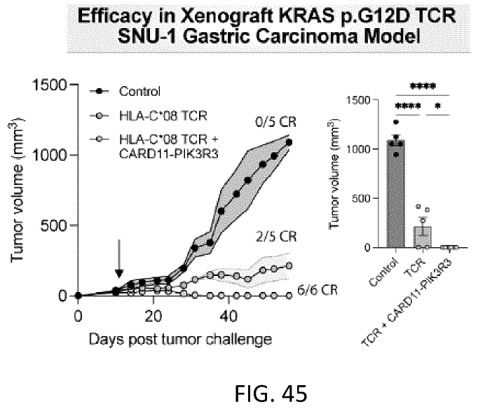

To further extend these TCR efficacy findings to human engineered TCR T cells, we developed a human TCR-based xenograft model. KRAS p.G12D, a common mutation present in human solid tumors, can be presented on various human HLA alleles and has been targeted by adoptive T cell therapies in small studies in humans. Utilizing HLA-C*08:02 over-expressing SNU-1 gastric carcinoma cells which harbor a KRAS p.G12D mutation and a clinically validated TCR against HLA-C*08:02 presented KRAS p.G12D, we observed significantly enhanced tumor clearance with CARD11-PIK3R3 expression (FIG. 45). Therefore, CARD11-PIK3R3 expression enhances the function of both human and mouse therapeutic TCR cells. Altogether, these data indicate that CARD11-PIK3R3 expressing T cells have superior therapeutic function in vivo in multiple immunotherapy refractory tumor models, including CAR and TCR-transgenic based models.

### Example 21

### CARD11-PIK3R3 Improves the Anti-Tumor Efficacy of Mouse CAR T Cells in a Syngeneic Mesothelioma Model

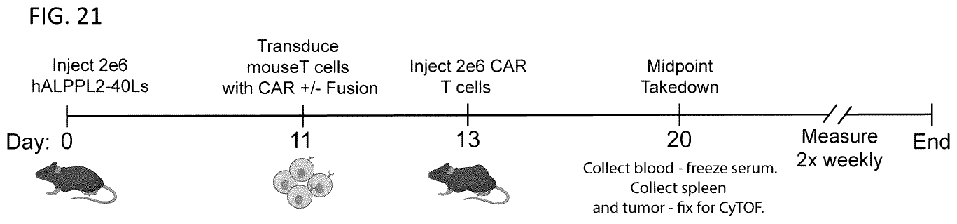

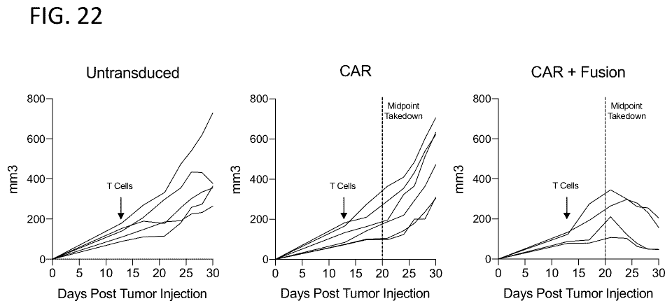

Mouse T cells were transduced with a hAPPL2-CAR, with or without the CARD11-PIK3R3 fusion (FIG. 21). T cells were transferred to mice bearing hAPPL2-40L mesothelioma tumors. In this model, C57BL/6J mice receive a subcutaneous injection of 2×10⁶ hALPPL2-expressing 40L (mesothelioma) tumor cells in the hind flank. T cells were collected from B6.SJL-Ptprca Pepcb/BoyJ (CD45.1) mice and transduced to express ALPPL2-BBz CAR with or without co-expression of CARD11-PIK3R3. Thirteen days after tumor inoculation, 2×10⁶ CAR+ mouse T cells were dosed to tumor bearing animals, and tumor growth was tracked via calipers measurements taken 3× weekly. Tumor volume was calculated using the following equation: (Length×Width×Width)/2, where length is the longest measurement. Mice were euthanized when the tumor reaches 2000 mm3 or 2 cm in either length or width. CARD11-PIK3R3 CAR T cells had significantly increased ability to control tumor volume (FIG. 22).

The approach used by the inventors/disclosers allowed the identification of a CARD11-PIK3R3 gene fusion which dramatically enhances therapeutic T cell function and efficacy through engaging the CBM complex. Furthermore, expression of the fusion enables superior tumor control at 100-fold lower cell doses than control cells in a fully immunocompetent murine model without lymphodepletion. Notably, the gene fusion the inventors/disclosers have identified would be inaccessible through previous T cell screening efforts such as loss of function, CRISPR activation, or wild-type gene overexpression screens. Therefore, naturally occurring mutations which have undergone positive selection in humans in vivo represent a novel and powerful set of tools to enhance cell therapy. The results obtained by the inventors/disclosers suggest that further testing of additional mutations identified in T cell lymphomas, or even other somatic mutations occurring in T cells including autoimmune disease, may hold promise for identifying additional approaches to enhance T cell function. Furthermore, this approach provides a platform for modifying other immune cell types with naturally occurring mutations for cell therapy, such as macrophages, NK cells, gamma delta T cells, or B cells. The potential utility of this approach is highlighted in the fact that while discovered in CD4+ T cells, the CARD11-PIK3R3 fusion dramatically enhances CD8+ T cell function.

The results presented herein implicate the CBM signalosome as a key regulator of therapeutic T cell function. Several individual outputs of CBM signaling, such as inducing AP-1 and NF-κB transcriptional activity and downregulation of MALT1 cleavage substrates REGNASE-1 and ROQUIN, have each been implicated independently as approaches to improve T cell therapy. This raises the possibility that a unifying feature of these various approaches is that they partly address a relative deficiency of CBM signaling. CARD11-PIK3R3 expression represents a powerful engineering solution to enhance each of these separate CBM outputs simultaneously. Further investigation of T cell lymphoma mutations in the context of adoptively transferred T cells may improve cellular therapies and elucidate new T cell biology.

### Example 22

### Safety in OT-I Mice

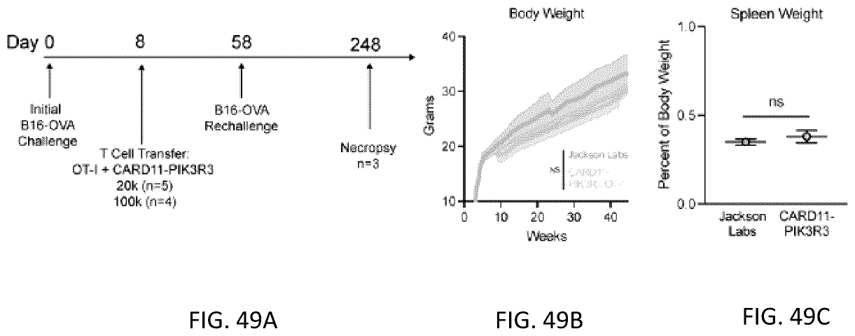

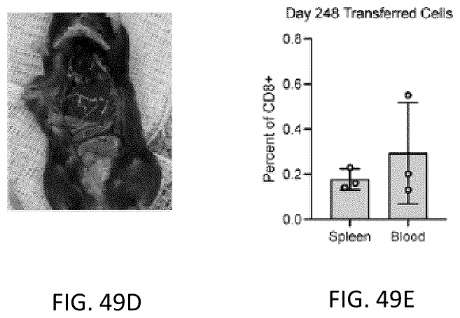

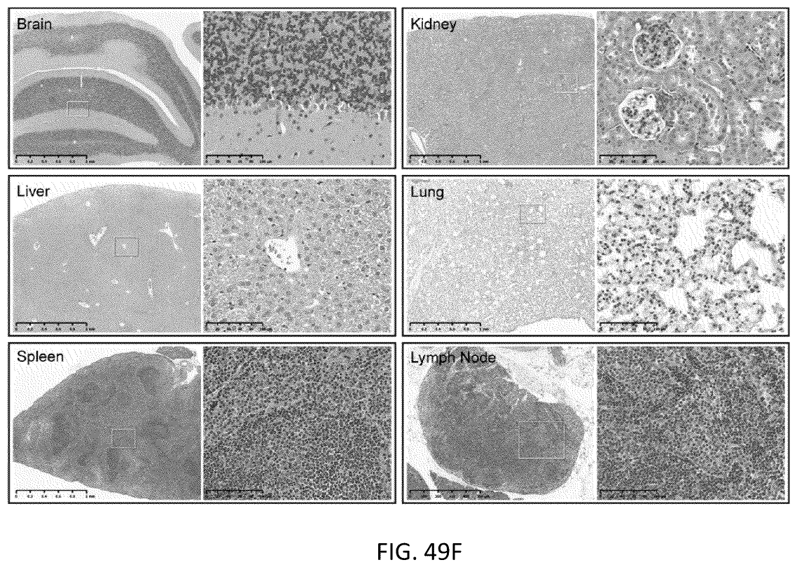

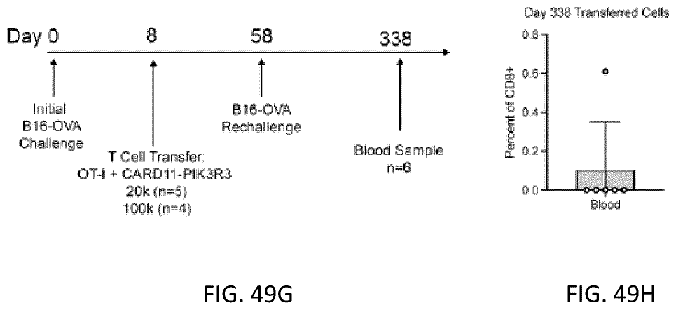

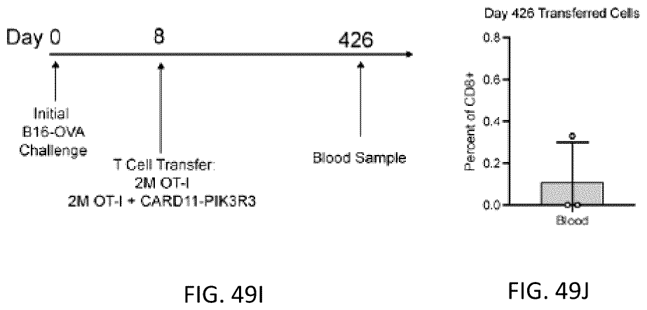

To address concerns of toxicity or transformation, we monitored the mice from FIG. 29 for up to 418 days after T cell transfer (FIG. 49A). Throughout this time, CARD11-PIK3R3 OT-I T cell treated animals gained weight similarly to that of published controls (FIG. 49B). To assess for occult disease, we performed necropsy on three mice on day 240 after T cell transfer. Their spleens were of normal weight and gross appearance (FIGS. 49C-49D). Additionally, CARD11-PIK3R3 OT-I T cells made up less than 1% of total CD8+ T cells in the spleen and blood (FIG. 49E). We performed hematoxylin and eosin (H&E) staining of common extra-nodal sites of lymphoma, as well as the spleen and lymph nodes. Pathology review failed to identify any evidence of nuclear atypia, destruction of normal cellular architecture, or neoplastic disease (FIG. 49F).

For the remaining 9 animals, we monitored the blood for evidence of leukemic disease for 330-418 after adoptive transfer. There were no atypical cells in the blood. Moreover, tail bleeds of CARD11-PIK3R3 OT-I treated animals revealed CARD11-PIK3R3 OT-I T cells were present at less than 1% of the overall CD8 population (FIGS. 49G-49J). This data suggests contraction of CARD11-PIK3R3 OT-I T cell population after exposure to initial or rechallenge tumors. Altogether, these data demonstrate a favorable safety profile of CARD11-PIK3R3 expressing anti-tumor T cells and a lack of evidence of malignant transformation in vivo over long time periods.

### Example 23

### STAT3 Putative Gain-of-Function Mutations

STAT3 is a transcription factor which is phosphorylated by receptor associated Janus kinases (JAKs). JAK/STAT signaling is critical for responses to signaling molecules such as cytokines. STAT3 is essential for T cell mediated control of infection. STAT3 plays an important role in T cell differentiation, including Th17 and Tfh differentiation. Additionally, STAT3 promotes the survival of T cells through inhibiting apoptosis (Oh et al. 2011. J. Biol. Chem. 286(35):30888-30897). Modulation of STAT3 signaling by expression of mutated versions of STAT3 may therefore be advantageous in the context of T cell therapies by modulation of one or more of the above mentioned roles of STAT3 in T cells.

STAT3 mutations tested include the following: STAT3_G618R, STAT3_N647I, and STAT3_D661I.

These mutations occurred in the SH2 domain. SH2 mutations in STAT3 can lead to increased STAT3 activity, as determined by phosphorylated STAT3 levels. SH2 mutations in STAT3 can also lead to cytokine independent growth of cell lines (Küçük et. al., 2015, Nature Communications. 6, Article number 6025)

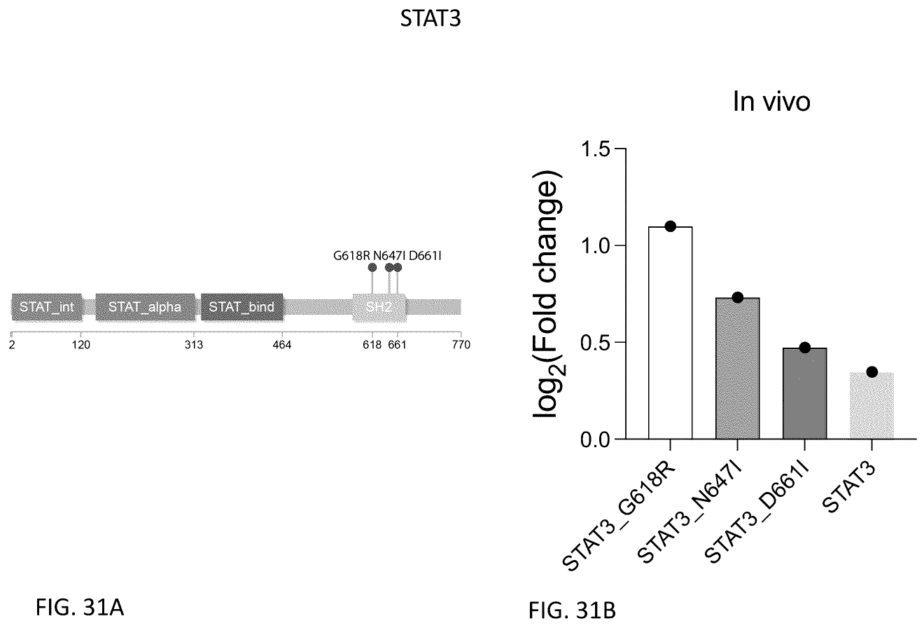

STAT3 mutations improved the in vivo accumulation and/or persistence of CAR T cells. In vivo screening identified positive log 2 fold change for all three STAT3 variants tested, suggesting that these mutations are advantageous for the in vivo accumulation and/or persistence of cell therapy (FIG. 31).

### Example 24

### BRAF Putative Gain-of-Function Mutations

BRAF is a serine/threonine kinase which plays a key role in regulating cell growth in response to signaling from receptor tyrosine kinases. The RAF/MEK/ERK pathway is essential for T cell development and T cell functions. This pathway is activated downstream of T cell receptor signaling. MEK/ERK signaling are required for IL-2 production and proliferation of naïve T cells in response to TCR stimulation. Therefore, modulation of this key signaling pathway in T cells may be advantageous to improving adoptive T cell therapies.

BRAF mutations tested include the following: BRAF_G469R, BRAF_D594N and BRAF_G469A.

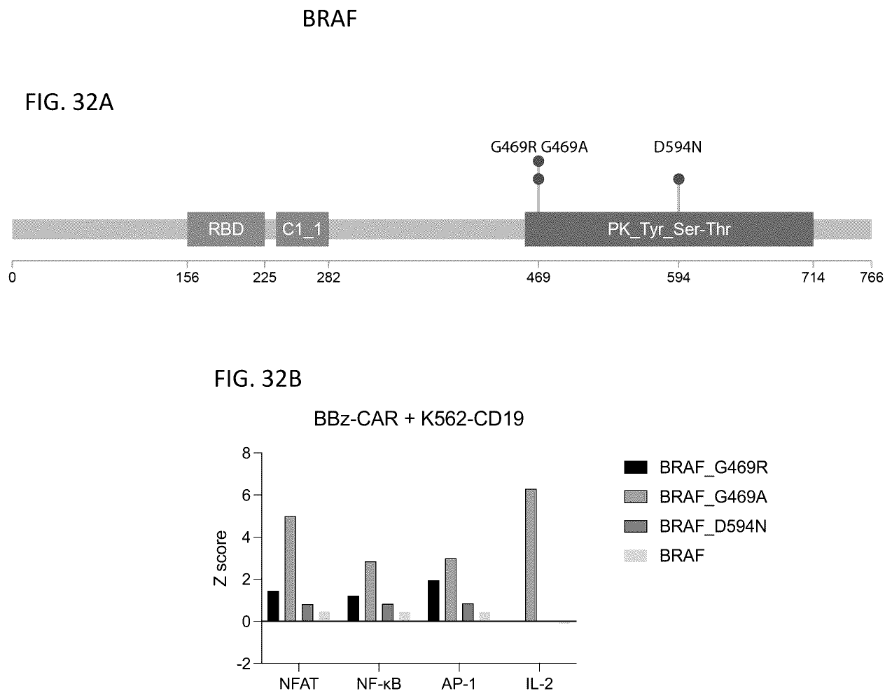

These mutations occurred in the BRAF kinase domain (FIG. 32). Kinase domain mutations in BRAF such as G469A can increase BRAF signaling as determined by kinase cascade (Davies, H. et. al. 2002. Nature. 417, 949-954)

BRAF mutations strongly enhanced CAR-dependent NFAT, NF-κB, and AP-1 signaling and IL-2 production (FIG. 32). Modulation of BRAF signaling by expression of BRAF mutations may therefore be used to alter CAR-T cell signaling.

### Example 25

### CARD11 Putative Gain-of-Function Mutations

CARD11 is a key signaling molecule in the CARD11-BCL10-MALT1 signalosome. CARD11-PIK3R3 fusion expression improves anti-tumor efficacy and enhances CARD11-BCL10-MALT1 signaling. Point mutations in CARD11 can similarly be used to enhance CARD11-BCL10-MALT1 signaling in therapeutic T cells.

CARD11 mutations tested include the following: CARD11_S615F, CARD11_D357N, CARD11_Y361C, and CARD11_E634K:S655C.

These mutations occurred in the inhibitory domain or the coiled coil domain. These mutations can increase NF-κB signaling and other CARD11-BCL10-MALT1 signalosome outputs (Da Silva Almeida et al. 2015. Nature Genetics. 47(12):1465-1470)

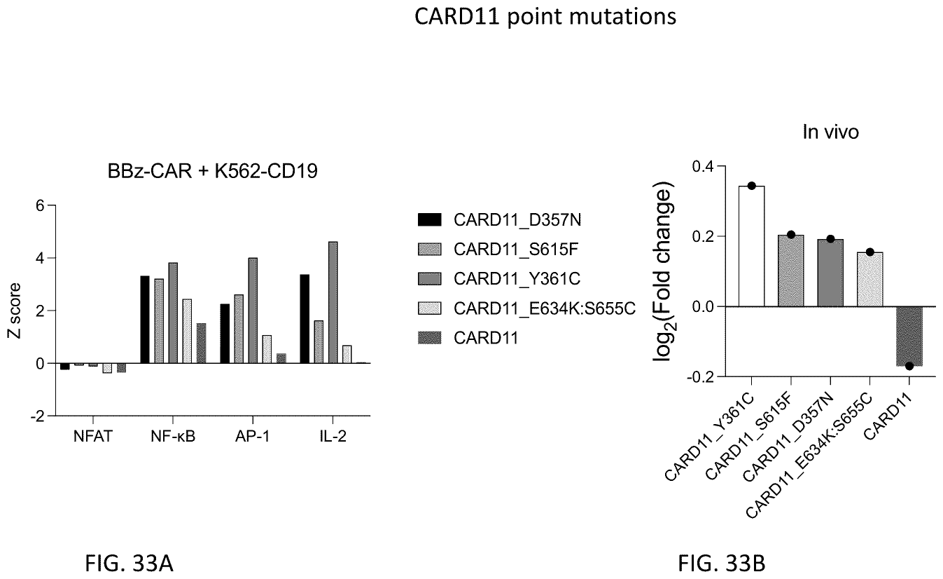

CARD11 mutations enhanced CAR-dependent NF-κB and AP-1 signaling and IL-2 production (FIG. 33). These mutations also increased the in vivo accumulation of CAR T cells. Modulation of CARD11-BCL10-MALT1 signaling by expression of CARD11 point mutations may therefore be used to alter CAR-T cell signaling and improve in vivo function.

### Example 26

### RASGRP1 Putative Gain-of-Function Mutation

RASGRP1 plays an important role in mediating TCR-dependent activation of Erk signaling. RASGRP1 plays a role in positive selection in the thymus and in TCR dependent T cell activation, as loss of RASGRP1 impairs these processes.

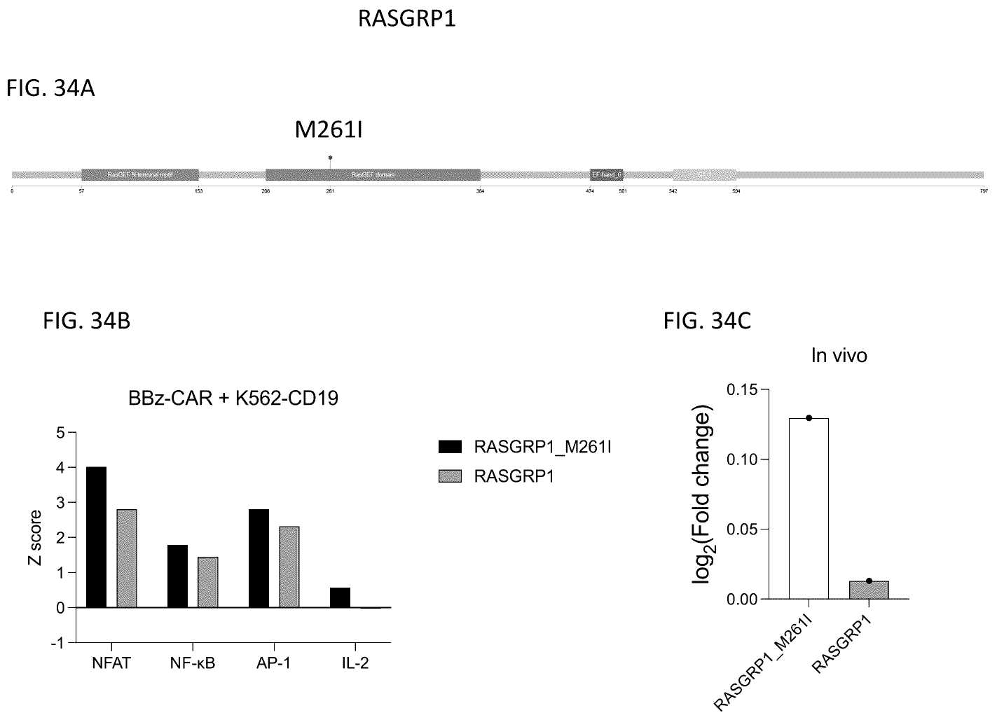

The RASGRP1 mutation tested was M261I. This mutation occurred in the RasGEF domain (FIG. 34). This domain is the catalytic domain of the protein which promotes guanyl nucleotide exchange.

RASGRP1 mutation enhances CAR-dependent NFAT, NF-κB and AP-1 signaling and IL-2 production compared to wild-type RASGRP1 (FIG. 34). This mutation also increased the in vivo accumulation of CAR T cells (FIG. 34). RASGRP1 mutation may therefore be used to alter CAR-T cell signaling and improve in vivo function.

### Example 27

### PLCG1 Putative Gain-of-Function Mutations

PLCG1 is a critical signaling molecule in T cells downstream of TCR activation. Upon activation, PLCG1 catalyzes the formation of IP3 and DAG from PI-4,5BP. IP3 and DAG in turn activate additional signaling cascades, including NF-κB and NFAT signaling. PLCG1 is required for the development, activation, proliferation, and cytokine production of T cells (Fu et al., 2010. J. Exp Med. 207(2):309-318)

The PLCG1 mutations tested include the following: PLCG1_D1165H, PLCG1_E1163K, PLCG1_E47K, PLCG1_R48W, PLCG1_S520F.

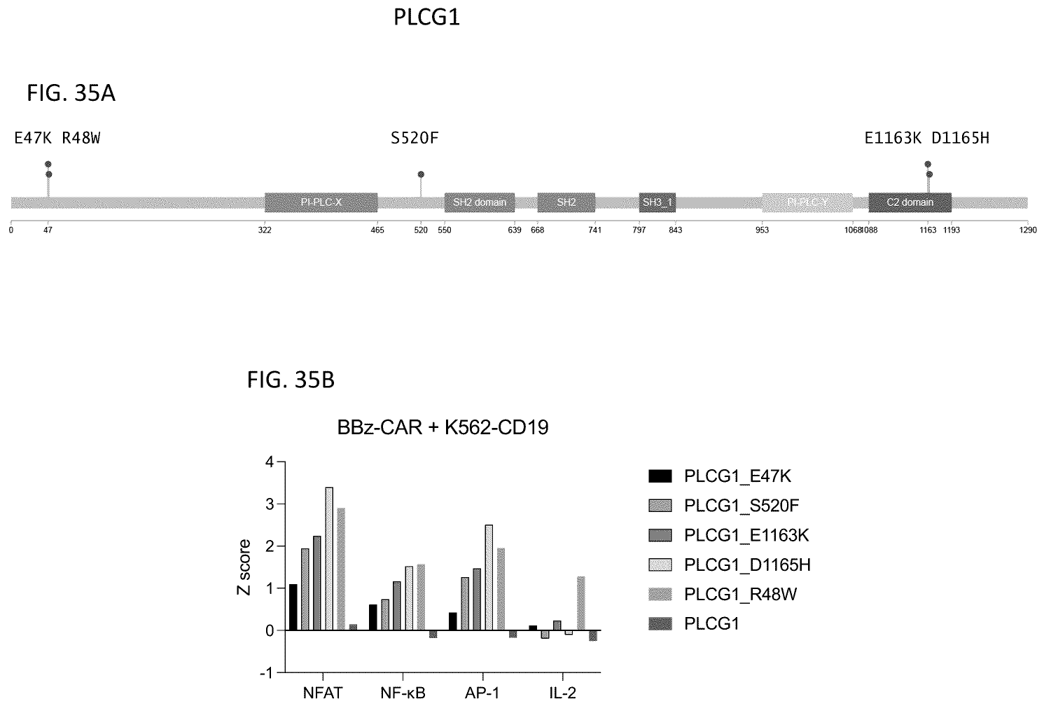

These mutations occurred in numerous regions of the PLCG1 protein including the C2 domain (FIG. 35). Several of these mutations have been previously characterized to activate NFAT, NF-κB, and AP-1 signaling basally (Fu et al., 2010. J. Exp Med. 207(2):309-318).

PLCG1 mutations enhanced CAR-dependent NFAT, NF-κB and AP-1 signaling and IL-2 production compared to wild-type PLCG1 (FIG. 35). PLCG1 mutation may therefore be used to alter CAR-T cell signaling.

### Example 28

### TNFRSF1B Putative Gain-of-Function Mutations

TNFRSF1B (TNFR2) is a co-stimulatory molecule expressed on activated T cells. TNFR2 binds to TNFa and LTa3. Upon binding, TNFR2 signals through TRAF2 to activate NF-κB signaling. TNFR2 is required for T cell expansion and effector differentiation (Ward-Kavanagh. 2016. Cell. Immunity. 44:1005-1019).

The TNFRSF1B mutations tested include TNFRSF1B_G256C and TNFRSF1B_T3771. The T377I mutation has been previously characterized to activate NF-κB signaling (Ungewickell et al. 2015. Nature Genetics. 47:1056-1060).

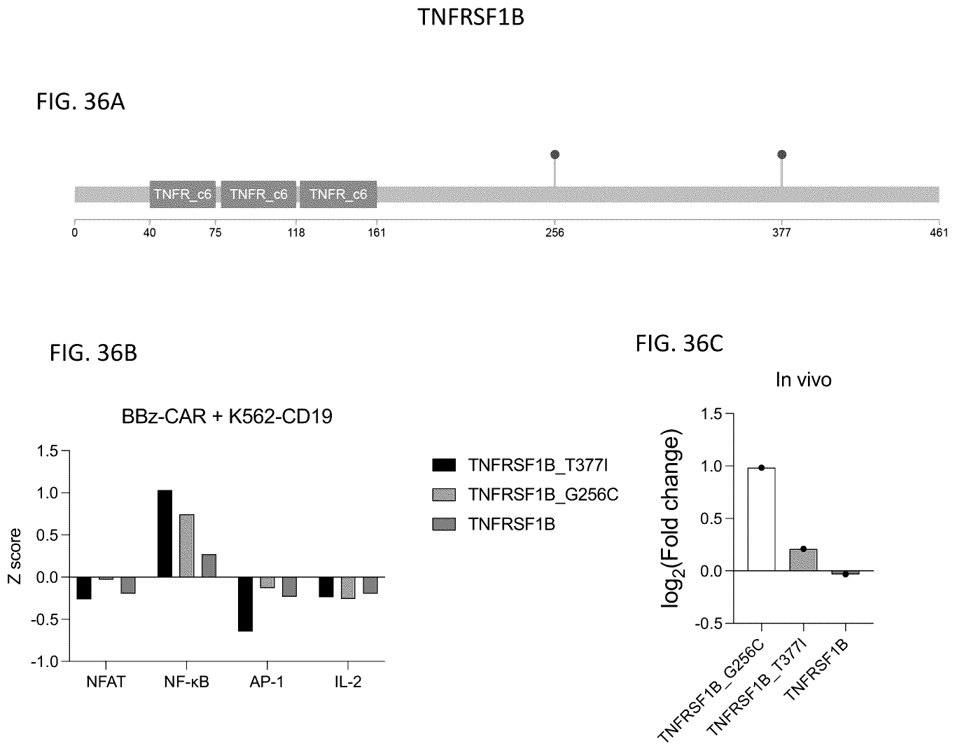

TNFRSF1B mutations activated CAR-dependent NF-κB to a greater extent than wild-type TNFRSF1B (FIG. 36). The TNFRSF1B mutations also led to higher in vivo accumulation than wild-type TNFRSF1B (FIG. 36). These mutations can therefore be leveraged to alter CAR signaling and enhance in vivo persistence.

### Example 29

### JAK Family Putative Gain-of-Function Mutations

JAK/STAT signaling plays a critical role in the biology of T cells, particularly in response to cytokines. Cytokine receptors that have recognized their ligand lead to the trans-activation of JAK proteins that are bound to the receptor. JAKs then phosphorylate STATs, which dimerize and translocate to the nucleus to affect transcription (Villarino et al. 2015. J. of Immunology. 194(1):21-27).

JAK/STAT signaling in T cells has previously been modified in numerous ways in order to potentiate cell therapies, through methods including synthetic cytokines, synthetic cytokine receptors including switch receptors, and through incorporation of JAK/STAT signaling domains in CAR constructs. The JAK1/JAK3 mutations tested include the following: JAK1_G1097A and JAK3_A573V.

The JAK3 mutation has previously been shown to increase STAT3 and STAT5 phosphorylation and the expansion of NK cells (Picod et al. 2022. Haematologica. 107(9):2218-2225).

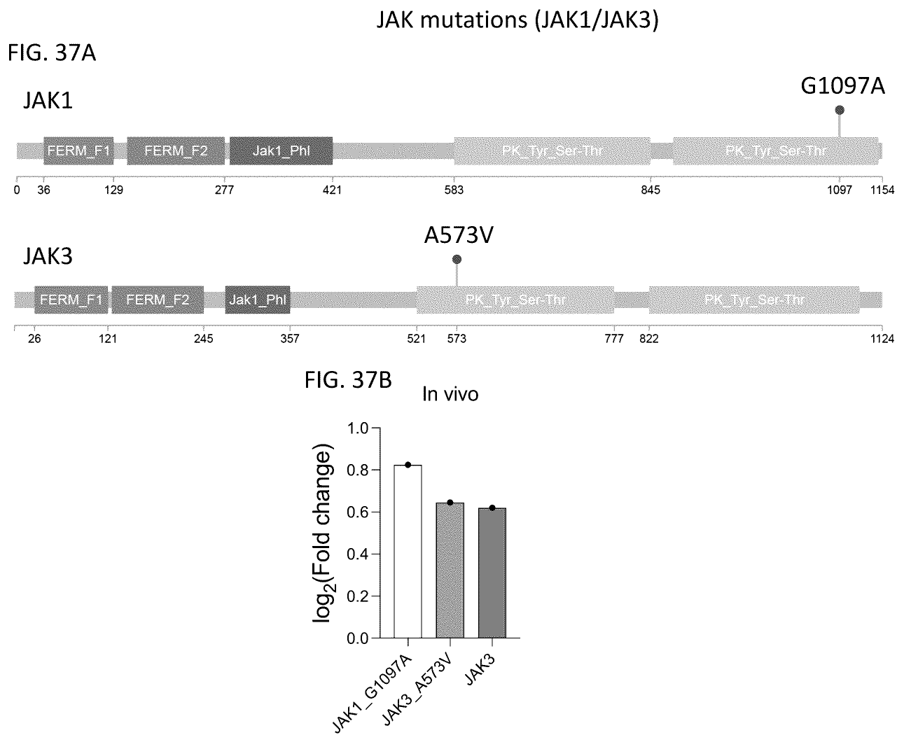

These mutations occurred in the kinase or pseudokinase domains of JAK1/JAK3 (FIG. 37). JAK1/JAK3 mutations had positive effects on in vivo accumulation (FIG. 37). These mutations can therefore be leveraged to enhance in vivo persistence in CAR T cells.

### Example 30

### Materials and Methods

### Receptor and Mutation Screening Construct Construction

Intracellular domains containing the appropriate costimulatory domain and CD3zeta domain were synthesized by Twist. Receptors were built by fusing the CD19 scFv1 to the corresponding receptor scaffold and intracellular tail. All receptors contain an n-terminal CD8a signal peptide (MALPVTALLLPLALLLHAARP SEQ ID NO:261) for membrane targeting and a flag-tag (DYKDDDDK SEQ ID NO: 262) for easy determination of surface expression with α-flag PE or BV421 (Biolegend 637310, 637322). In some cases the receptors additionally contained a T2A self-cleaving sequence followed by a tNGFR sequence, used in downstream applications for T cell isolations. The receptors were cloned into a modified pHR′SIN:CSW vector containing a PGK promoter for all primary T cell experiments.

### Primary Human T Cell Isolation and Culture

Primary CD3+, CD4+ and CD8+ T cells were isolated from anonymous donor blood after apheresis by negative selection (Biolegend Mojo Sort #480129, 480130, 480131). Blood was obtained from Blood Centers of the Pacific (San Francisco, CA) as approved by the University Institutional Review Board. T cells were cryopreserved in RPMI-1640 (UCSF cell culture core) with 20% human AB serum (Valley Biomedical Inc., #HP1022) and 10% DMSO. After thawing, T cells were cultured in human T cell medium consisting of X-VIVO 15 (Lonza #04-418Q), 5% Human AB serum and 10 mM neutralized N-acetyl L-Cysteine (Sigma-Aldrich #A9165) supplemented with 30 units/mL IL-2 (NCI BRB Preclinical Repository) for all experiments unless otherwise noted. In vivo experiments were completed with bulk CD3+ cells isolated in a similar manner.

### Lentiviral Transduction of Human T Cells

Pantropic VSV-G pseudotyped lentivirus was produced via transfection of Lenti-X 293T cells (Clontech #11131D) with a pHR′SIN:CSW transgene expression vector and the viral packaging plasmids pCMVdR8.91 and pMD2.G using Mirus TransIT-Lenti (Mirus #MIR 6606). Primary T cells were thawed the same day, and after 24 hours in culture, were stimulated with Human T-Activator CD3/CD28 Dynabeads (Life Technologies #11131D) at a 1:3 cell:bead ratio. At 48 hours, viral supernatant was harvested and the primary T cells were exposed to the virus for 24 hours. At day 5 post T cell stimulation, the Dynabeads were removed, T cells were sorted, and the T cells expanded until day 10-14 when they were rested and could be used in vitro or in vivo assays. T cells were sorted for assays with a Beckton Dickinson (BD) FACs ARIA II.

### TCR Knockout and Lentiviral Transduction of Human T Cells

For TCR KO experiments, primary T cells were cultured in human T cell medium consisting of X-VIVO 15 (Lonza #04-418Q), 5% Human AB serum and 10 mM neutralized N-acetyl L-Cysteine (Sigma-Aldrich #A9165) supplemented with 100 units/mL IL-7 (Miltenyi #130-095-362) and 100 units/mL IL-15 (Miltenyi #130-095-765). Primary T cells were thawed, rested for 1 hour then stimulated with Human T-Activator CD3/CD28 Dynabeads (Life Technologies #11131D) at a 1:3 cell:bead ratio. One day after activation, T cells were transduced with concentrated virus. 24 hours later, virus and Dynabeads were removed, cells were rested for 24 hours, then resuspended at 1×100⁶ cells per mL in P3 electroporation buffer (Lonza #V4SP-3960) with gRNA (CAGGGTTCTGGATATCTGT) targeting the human TRAC locus and Cas9. 23 μL of this mixture was aliquoted to each well of a 96 well nucleofection plate (Lonza #V4SP-3960) and immediately electroporated using a 4-D Lonza Nucleofector with program EH115. Cells were resuspended in pre-warmed human T cell medium and recovered for 30 minutes in the incubator before being transferred to culture. Electroporated cells were assessed for TCR KO and lentiviral transduction via flow before injection to mice.

### Cell Lines

This Example describes the various cell lines used in the experiments of this study. The cancer cell lines used were K562 myelogenous leukemia cells (ATCC #CCL-24 3), Jurkat cells (Clone E6-1, ATCC #TIB-152), B16-F10 melanoma cells (ATCC #CRL-6475) and A549 lung epithelial carcinoma cells (ATCC #CCL-18). K562s and A549s were lentivirally transduced to stably express human CD19. CD19 levels were determined by flow cytometry (Biolegend, clone HIB19). A549s were additionally transduced to express the nuclear stain mkate2. All cell lines were sorted for expression of the transgenes. Jurkat cells were transduced with retroviruses encoding fluorescent reporter constructs (Addgene 118095, 118094, and 118031) and stimulated with PMA/ionomycin to sort a line with high induction of each reporter. To enable screening with our mCherry expressing lentiviral constructs, an iRFP fluorescent reporter was subcloned to replace mCherry in the previously described AP1-mCherry construct. Triple reporter cells were then transduced with a CD19-CD28z CAR or CD19-BBz-CAR. Jurkat and K562 cells were cultured in RPMI+10% FBS with penicillin/streptomycin and sodium pyruvate. B16-OVA and A549 cells were cultured in DMEM+10% FBS with penicillin/streptomycin. All cell lines were routinely tested for mycoplasma contamination (Southern Biotech).

### In Vitro Primary T Cell Assays

For in vitro co-culture assays, transduced primary T cells were co-cultured with target cells at effector to target ratios and co-culture time courses. For Luminex assays, cells were co-cultured in media lacking exogenous IL-2, supernatants were collected 48 hours after start, frozen at −80C and sent for analysis to Eve Technologies. For IL-2 ELISA assays, supernatant was collected from 24-hour co-cultures of transduced T cells and target cells. Supernatants were assessed for IL-2 secretion using the Invitrogen Instant IL-2 ELISA kit (Invitrogen #BMS221INST). For repeat stimulation assay, transduced T cells were co-cultured on the adherent CD19 expressing A549 cell line, after 1 week T cells were removed from co-culture without disturbing adherent cells, counted and replated on CD19 expressing A549 cells seeded 24 hours prior. For co-culture assays performed over a period of days to weeks, media was supplemented regularly. For Mass cytometry by time of flight (CyTOF) transduced CD3+ T cells were co-cultured with CD19-K562s for 7.5, 15, 30, 60 or 120 minutes, then immediately fixed with 1.6% PFA for 10 minutes at room temperature. Cells were then pelleted, washed, and stored at −80C until ready for barcoding and staining. All primary T cell in vitro assays were performed with three donors.

### Flow Cytometry

For all in vitro primary T cell assays, cells were washed with PBS 2% FBS twice, stained with surface staining markers at room temperature for 20 minutes, washed twice, and resuspended in PBS 2% FBS with DRAQ7 (diluted 1:1000) before analysis on a BD FACSymphony X-50 Flow Cytometer. The following antibodies were used: CD8 (SK1), CD25 (M-A251), CD39 (A1), CD69 (FN50), CD271 (ME20.4), CD278 (C398.4A), CD279 (EH12.2H7)

### K562 In Vivo Xenograft Assays

NOD.Cg-Prkdcscid Il2rgtm1Wjl/SzJ (NSG) mice were dosed with 1×10⁶ CD19 expressing K562 cells via subcutaneous injection. 5 days post tumor injection, 3 to 6×10⁶ transduced or control T cells were dosed to tumor bearing animals via retro-orbital injection. Tumors were measured with calipers twice weekly, and tumor volume was calculated using the following formula: (length×width2)/2. Throughout experiment animal drinking water was supplemented with Clavomox to prevent bacterial infections.

### Nalm6 In Vivo Xenograft Assays

NOD.Cg-Prkdcscid Il2rgtm1Wjl/SzJ (NSG) mice were dosed with 5×10⁵ luciferase expressing Nalm6 cells via intravenous tail vein injection. 5 days post tumor injection, 1×10⁶ transduced or control T cells were dosed to tumor bearing animals via retro-orbital injection. Tumor burden was measured twice weekly via bioluminescence imaging after 200 uL intraperontineal injection of D-Luciferin (15 mg/mL, Goldbio), image analysis performed using Living Image (Perkin-Elmer). Animal drinking water was supplemented with Clavomox to prevent bacterial infections.

### Jurkat Reporter Screening and Analysis

CAR triple reporter Jurkat cells were transduced in 96 well plate format with individual T cell lymphoma mutation lentiviruses. 48 hours post-transduction, cells were plated at approximately a 1:1 ratio with K562 or K562-CD19 cells. Plates were spun at 300 g for 2 minutes to promote interaction of CAR cells and target cells. Following 24 hours of co-culture, supernatants were removed for IL-2 ELISA (ELISA MAX™ Deluxe Set Human IL-2, Biolegend) and cells were washed in FACS buffer (PBS+2% FBS) and analyzed by FACS. For the CD19-BBz CAR screen, cells were stained with anti-PD-1 (Biolegend, clone EH12.2H7) prior to FACS analysis. Each screen was performed in biological replicate with separate lentiviral transductions. For analysis, the percentage positive for each reporter in each condition (i.e. each CAR and co-culture cell type) was compared to the mCherry only controls and the corresponding wild-type gene control when available by T test followed by Bonferroni correction. Z scores were calculated for each condition as (x−μ)/σ (where x is the observed value, μ is the mean, and σ is the standard deviation).

### In Vivo Screening and Analysis

Human CD3 T cells were lentivirally co-transduced with a CD19-BBZ CAR with tNGFR and the mutant construct library in an arrayed fashion. CD3+ T cells were assessed via flow for CD19-BBZ CAR (FLAG) and mutant construct (mCherry) expression. T cells were pooled based on mCherry expression and sorted for a purified dual positive population. 6×106 library T cells were injected to 15 CD19-K562 tumor bearing animals. T cells were isolated from the tumor and spleen by positive selection using the tNGFR (CELLection™ Biotin Binder Kit) at 7, 14, and 21 days post injection. gDNA was isolated from these T cells (NucleoSpin Tissue, Machery Nagel), and quantified by NanoDrop Spectrophotometry and adjusted to 8 ng/uL. PCR amplification of the samples was performed using primers (Table 2) to enrich for the mutations barcodes (PCR1). PCRs were performed with Kappa HiFi, in reaction volumes of 50 μL with between 10 and 1090 ng of gDNA, all reactions underwent 7 cycles of amplification. For the subsequent PCR to add Illumina barcodes and adapters to the products (PCR2), all products from PCR1 were quantified with a Qubit 1×dsDNA High Sensitivity Assay kit (Invitrogen) and up to 10 ng of template was used in a 25 μL reaction with Kappa Hifi. Different forward and reverse primers were used for each sample for PCR2 to add unique custom Illumina 15 and 17 barcode sequences to each sample. Finally, PCR2 products were again quantified using Quibit, and these products were pooled at 1:1 molar ratio, diluted, loaded, and run on a MiniSeq 75 cycle cartridge using the standard manufacturer protocols.

### Bulk RNAseq and Analysis

For bulk RNAseq, T cells from 3 independent healthy donors transduced with the indicated constructs were either unstimulated or stimulated via co-culture with CD19 expressing A549 cells for 8 hours. RNA was then isolated (Nucleospin RNA XS, Machery-Nagel and cDNA libraries were constructed using SMART-Seqv4 Ultra Low Input RNA Kit (Takara Bio) and Nextera XT (Illumina). Sequencing reads were aligned using STAR, transcripts quantified using HT-Seq, and differentially expressed genes were identified using DESeq2 as previously described. (Love M I, Huber W, Anders S. Moderated estimation of fold change and dispersion for RNA-seq data with DESeq2. Genome Biol. 2014; 15(12):1-21.)

### Immunoblot Analysis

Jurkat cells (1-5×10⁶) were pre-treated for 30 minutes with the MALT1 inhibitor Z-VRPR-FMK (75 uM) or vehicle control. Cells were then treated with PMA/ionomycin as indicated for 2 hours. Whole-cell lysates were generated and analyzed by immunoblotting with the following antibodies: anti-HOIL1 (Millipore Sigma, MABC576), anti-CYLD (Santa Cruz, sc-74435), and anti-3-actin (Cell Signaling Technologies, 4967).

### Mass Cytometry by Time of Flight (CyTOF)

Cells were thawed on ice for 15 minutes, washed, and barcoded with distinct combinations of stable Pd isotopes for 15 min at room temperature on a shaker in Maxpar Barcode Perm Buffer (Fluidigm, cat #201057) (Mass tag cellular barcodes; Bodenmiller B, Zunder E R, Finck R, et al. Multiplexed mass cytometry profiling of cellular states perturbed by small-molecule regulators. Nat Biotechnol 2012 309. 2012; 30(9):858-867). Cells were washed twice with cell staining media (PBS with 0.5% BSA and 0.02% NaN3) and pooled into a single cluster tube. Cells were stained with surface staining antibodies for 30 min at room temperature on a shaker. After staining, cells were washed two times with cell staining media followed by permeabilization with methanol for 10 min at 4° C. Permeabilized cells were washed twice in cell-staining media and stained with intracellular antibodies for 30 min at room temperature on a shaker. Cells were washed twice in cell-staining media and then stained with 1 ml of 1:4000 191/193Ir DNA intercalator (Fluidigm) diluted in PBS with 1.6% PFA overnight. Cells were diluted in Cell Acquisition Solution containing bead standards (Fluidigm, Cat #201078) to and analyzed on a Helios mass cytometer (Fluidigm) equilibrated with Cell Acquisition Solution. After acquisition, the data from the pooled samples underwent bead standard data normalization and de-barcoding using the R package from the PICI institute available at https://github.com/ParkerICI/premessa.

### Mouse T Cell Purification and Retroviral Transduction

CD8+ T cells were isolated from the spleens of CD45.1+ or CD45.2 OT-J mice using a mouse CD8+ T cell isolation kit (Stemcell Technologies) or Mouse Pan CD3+ T cell isolation kit (Biolegend). T cells were then cultured with 100 U/mL recombinant human IL-2 (Peprotech), anti-CD3e (1 ug/mL) and anti-CD28 (0.5 ug/mL) overnight. For CAR experiments, T cells were stimulated overnight with anti-CD3/CD28 beads (ThermoFisher). Retroviral supernatants were added to T cells in Retronectin (Takara) coated plates and spinduction was performed for 1 hour at 2000 rpm at 30° C. Following transduction, T cells were resuspended and cultured in fresh media containing 100 U/mL IL-2 until adoptive transfer. Transduction efficiency was determined by flow cytometry prior to adoptive transfer.

### B16-OVA Melanoma Tumor Model

Female C57BL/6 mice age 6-8 weeks were injected subcutaneously with 5×10⁵ B16-OVA melanoma cells. Mice with similar sized tumors were randomized to receive treatments of OT-J T cells. 100 μL of T cells (or PBS control) were retro-orbitally injected on day 8-12 post tumor inoculation. For dual-transfer competitive assay and tumor infiltrating lymphocyte analysis experiments, 1×10⁶ T cells were transferred. For anti-tumor efficacy, 2×10⁶ T cells were transferred. Tumor lengths and widths were determined every 2-3 days by digital caliper measurement and tumor volume was calculated as length×width×[(length×width)0.5]×π/6 as previously described (Wei, J., Long, L., Zheng, W. et al. Targeting REGNASE-1 programs long-lived effector T cells for cancer therapy. Nature 576, 471-476 (2019). https://doi.org/10.1038/s41586-019-1821-z).

Death was defined as a progressively growing tumor that reaches 15 mm in its longest axis or developing ulceration or necrosis. For the anti-tumor efficacy experiment, measurement of tumors and determination of survival endpoint was blinded to experimental condition. Experiments were performed in accordance with Northwestern University Institutional Animal Care and Use Committee approved protocols.

### B16-OVA Tumor Infiltrating Lymphocyte (TIL) Isolation and Analysis

To isolate TILs, B16-OVA tumors were excised, minced, and digested using collagenase IV (1 mg/mL) and DNAse I (50 ug/mL) for 30 minutes at 37° C. in a shaking incubator at 200 rpm. TILs were filtered through a 70 uM cell strainer and isolated over Percoll density centrifugation. For dual-transfer experiments, the isolated TILs were subjected to Fc receptor blocking, live/dead staining (Live/Dead Violet) and surface marker staining for flow cytometric analysis. For intracellular cytokine staining, cells were restimulated ex vivo in media containing brefeldin and monensin (Invitrogen), phorbol myristate acetate, and ionomycin for four hours. Following Fc blocking, live/dead staining, and cell surface marker staining, cells were fixed (BD CytoFix), permeabilized (Invitrogen 10× Perm), and stained for intracellular proteins.

### CD19-B16 Melanoma Tumor Model

Male B6.SJL-Ptprca Pepcb/BoyJ mice age 6-12 weeks were injected subcutaneously with 1×10⁵ CD19-B16 melanoma cells. Mice with similar sized tumors were randomized to receive treatments of CAR T cells. 2×10⁶ CAR+ or untransduced (control) T cells were retro-orbitally injected on day 12 post tumor inoculation. Tumor lengths and widths were determined every 2-3 days by digital caliper measurement and tumor volume was calculated using the following formula: (length×width2)/2. Death was defined as a progressively growing tumor that reaches, 2000 mm3 or 20 mm in its longest axis, whichever comes first.

### ALPPL2-40L Mesothelioma Tumor Model

Male B6.SJL-Ptprca Pepcb/BoyJ mice age 6-12 weeks were injected subcutaneously with 2×10⁶ ALPPL2-40L mesothelioma cells. Mice with similar sized tumors were randomized to receive treatments of CAR T cells. 2×10⁶ CAR+ or untransduced (control) T cells were retro-orbitally injected on day 12 post tumor inoculation. Tumor lengths and widths were determined every 2-3 days by digital caliper measurement and tumor volume was calculated using the following formula: (length×width2)/2. Death was defined as a progressively growing tumor that reaches, 2000 mm3 or 20 mm in its longest axis, whichever comes first.

### CARD11 and BCL10 CRISPR Knock-Out

CARD11 and BCL10 CRISPR knock-out CRISPR knockout was performed in triple reporter Jurkat cells using the SE Cell Line 4D Nucleofector Kit (Lonza). 1×10⁶ cells were nucleofected with Cas9 only or ribonuclear protein complexes of Cas9 with CARD11 gRNA (CAATGACCTTACACTGACGC SEQ ID NO: 263) or BCL10 gRNA (TCGCCGAATAGATTCAACAA SEQ ID NO: 264).

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

TABLE 2
LIST OF PCR1 PRIMERS

| | |
|---|---|
| PCR1F_1 | TCGTCGGCAGCGTCAGATGTGTATAAGAGACAGacgtagaagagaatcctgggc |
| PCR1F_2 | TCGTCGGCAGCGTCAGATGTGTATAAGAGACAGNacgtagaagagaatcctgggc |
| PCR1F_3 | TCGTCGGCAGCGTCAGATGTGTATAAGAGACAGNNacgtagaagagaatcctgggc |
| PCR1F_4 | TCGTCGGCAGCGTCAGATGTGTATAAGAGACAGNNNacgtagaagagaatcctgggc |
| PCR1F_5 | TCGTCGGCAGCGTCAGATGTGTATAAGAGACAGNNNNacgtagaagagaatcctgggc |
| PCR1R_1 | GTCTCGTGGGCTCGGAGATGTGTATAAGAGACAGttggtcaccttcagcttggc |
| PCR1R_2 | GTCTCGTGGGCTCGGAGATGTGTATAAGAGACAGNttggtcaccttcagcttggc |
| PCR1R_3 | GTCTCGTGGGCTCGGAGATGTGTATAAGAGACAGNNttggtcaccttcagcttqqc |
| PCR1R_4 | GTCTCGTGGGCTCGGAGATGTGTATAAGAGACAGNNNttggtcaccttcagcttggc |
| PCR1R_5 | GTCTCGTGGGCTCGGAGATGTGTATAAGAGACAGNNNNttggtcaccttcagcttggc |

TABLE 3
RESULTS OF IN VIVO MUTATION SCREENING

| Construct | In vivo log2 fold change |
|---|---|
| CARD11-PIK3R3 | 3.0005 |
| MYCN_P44L | 2.5543 |
| CCND3_P284S | 2.0288 |
| MYCN | 1.7046 |
| GATA3_Y63X | 1.1711 |
| CCND3 | 1.1358 |
| STAT3_G618R | 1.0989 |
| PLCG1 | 1.0924 |
| TNFRSF1B_G256C | 0.98454 |
| CARMIL2_Q575E | 0.95534 |
| RHOA_C16R | 0.83989 |
| JAK1_G1097A | 0.82419 |
| PLCG1_S520F | 0.81648 |
| CARMIL2 | 0.81511 |
| KCNQ1 | 0.74451 |
| STAT3_N647I | 0.73129 |
| mCherry | 0.71787 |
| JAK3_A573V | 0.64517 |
| JAK3 | 0.6204 |
| SMARCB1 | 0.61464 |
| NPM-TYK | 0.61211 |
| PRKCB1 | 0.60187 |
| ITK-SYK | 0.57059 |
| CD53 | 0.55832 |
| LATS1_P165T | 0.55327 |
| NFKB2_K656X | 0.54768 |
| CD3E_S41C | 0.49715 |
| STAT3_D661I | 0.47289 |
| VAV1 | 0.46373 |
| ITK-FER | 0.45221 |
| PRKCB1_D427N | 0.42466 |
| VAV1_R798Q | 0.38758 |
| NFKB1 | 0.36173 |
| EIFS1 | 0.35107 |
| STAT3 | 0.34767 |
| KCNQ1_R583C | 0.34574 |
| CARD11_Y361C | 0.34369 |
| ECSIT_V140A | 0.30825 |
| TBL1XR1 | 0.3019 |
| EIFS1_R89I | 0.294 |
| RARA | 0.29369 |
| PDCD1_R231X | 0.26309 |
| TP53_R273P | 0.2616 |
| NFKB1_H67Y | 0.23355 |
| TNFRSF1B_T377I | 0.20846 |
| DGKA | 0.20622 |
| CARD11_S615F | 0.20484 |
| CARD11_D357N | 0.19285 |
| CARD11_E634K: S655C | 0.15507 |
| CSNK1A1_S27C | 0.14252 |
| RHOA | 0.1348 |
| CD28_T195P | 0.13182 |
| RASGRP1_M261I | 0.1296 |
| NRAS | 0.067501 |
| CSNK1A1 | 0.061897 |
| TP53 | 0.043614 |
| RASGRP1 | 0.013008 |
| BRAF | −0.0035971 |
| IRF4 | −0.010882 |
| JUNB | −0.025732 |
| TNFRSF1B | −0.032402 |
| ECSIT | −0.12417 |
| SPI1-TCF7 | −0.12925 |
| PRKCB1_D630Y | −0.13355 |
| NFKB2 | −0.15157 |
| VAV1_D797H | −0.15363 |
| RHOA_G17V | −0.16445 |
| CARD11 | −0.16964 |
| CSNK2B_Q182X | −0.17317 |
| CD28_Q77P | −0.17518 |
| GNAQ | −0.19753 |
| CD28 | −0.22358 |
| NRAS_Q61H | −0.23431 |
| BCOR | −0.23446 |
| FYN | −0.24392 |
| CSNK2B | −0.27225 |
| IRF4_K59R | −0.27748 |
| BRAF_G469R | −0.28717 |
| ITGB2 | −0.28743 |
| SMARCB1_Q368X | −0.28979 |
| PLCG1_E1163K | −0.33003 |
| CD3E | −0.34196 |
| TBL1XR1_H307R | −0.36523 |
| FYN_Y531H | −0.38263 |
| BRAF_D594N | −0.42484 |
| BCL6_S647R | −0.50434 |
| CD28-CTLA4 | −0.51046 |
| CD28_F51I | −0.52077 |
| GNAQ_T96S | −0.5295 |
| NPM-ALK | −0.54833 |
| BRAF_G469A | −0.56155 |
| PLCG1_E47K | −0.68232 |
| STAT5B_T628S | −0.68373 |
| CD28_F51V | −0.68501 |
| STAT5B | −0.82356 |
| CBLB | −0.85465 |
| ITGB2_E234K | −0.93692 |
| BCOR_N1459S | −1.0945 |
| MSC_E116K | −1.1425 |
| DGKZ | −1.183 |
| VAV1_E556D | −1.2374 |
| PLCG1_R48W | −1.3029 |
| PLCG1_D1165H | −1.3605 |
| VAV1-GSS | −1.3956 |
| ICOS-CD28 | −1.5123 |
| STAT5B_Y665F | −1.5667 |
| RARA_G206S | −1.8206 |
| SELENOI-ABL1 | −2.1187 |
| GATA3 | −2.1676 |
| JUNB_A282V | −2.2295 |
| MSC | −2.9114 |
| BCL6 | −3.088 |
| FYN_R86G | −4.9583 |
| PDCD1 | −5.2934 |

TABLE 4
RESULTS OF IN VITRO MUTATION SCREENING

| Construct | condition | group | CAR | nfat_zscore | nfkb_zscore | ap1_zscore | il2_zscore | pd1_zscore |
|---|---|---|---|---|---|---|---|---|
| BCL6 | K562 | Wild-type | 28z | −0.04037972 | −0.0955831 | −0.41976843 | NA | NA |
| BCL6 | K562 | Wild-type | BBz | −0.42494348 | −0.14672375 | −0.48966525 | NA | −0.14045184 |
| BCL6 | K562-19 | Wild-type | 28z | −0.60059827 | −0.70834113 | −0.81444001 | −0.34152766 | NA |
| BCL6 | K562-19 | Wild-type | BBz | −0.18288789 | −0.11036598 | −0.65804064 | −0.21448862 | 0.53255809 |
| BCL6_S647R | K562 | Mutant | 28z | −0.35915673 | −0.29389965 | −0.30193543 | NA | NA |
| BCL6_S647R | K562 | Mutant | BBz | −0.69736303 | −0.63294304 | −0.27092471 | NA | −0.15216875 |
| BCL6_S647R | K562-19 | Mutant | 28z | −1.4472224 | −0.87572869 | −0.06785586 | −0.34164038 | NA |
| BCL6_S647R | K562-19 | Mutant | BBz | −1.0354498 | −0.36073958 | −0.23781412 | −0.2349297 | −0.06574864 |
| BRAF | K562 | Wild-type | 28z | 0.41734257 | −0.12651894 | 0.53507192 | NA | NA |
| BRAF | K562 | Wild-type | BBz | −0.15086105 | −0.5089276 | −0.22431594 | NA | −0.14240422 |
| BRAF | K562-19 | Wild-type | 28z | 1.29334332 | 0.51574434 | 1.23532286 | −0.07349989 | NA |
| BRAF | K562-19 | Wild-type | BBz | 0.4989266 | 0.48421561 | 0.47549493 | −0.11387979 | 0.07579773 |
| BRAF_D594N | K562 | Mutant | 28z | 0.23749219 | −0.06506305 | 0.23140873 | NA | NA |
| BRAF_D594N | K562 | Mutant | BBz | −0.90612685 | −0.69591963 | −0.03449949 | NA | −0.13537424 |
| BRAF_D594N | K562-19 | Mutant | 28z | 1.17145567 | 1.15905427 | 0.95419286 | 0.38641353 | NA |
| BRAF_D594N | K562-19 | Mutant | BBz | 0.83238883 | 0.83616754 | 0.85878988 | −0.01315878 | 0.05440468 |
| BRAF_G469A | K562 | Mutant | 28z | 4.19343309 | 2.29844317 | 8.27352879 | NA | NA |
| BRAF_G469A | K562 | Mutant | BBz | 4.53554578 | 3.36214342 | 8.97191503 | NA | 0.13955362 |
| BRAF_G469A | K562-19 | Mutant | 28z | 5.687353 | 2.32667926 | 3.05616511 | 5.55025808 | NA |
| BRAF_G469A | K562-19 | Mutant | BBz | 5.00458398 | 2.85478237 | 3.00208717 | 6.30688486 | −0.10690446 |
| BRAF_G469R | K562 | Mutant | 28z | 1.52300962 | 0.08415717 | 1.0106096 | NA | NA |
| BRAF_G469R | K562 | Mutant | BBz | −0.34720489 | −0.75881493 | 0.2378824 | NA | −0.12170792 |
| BRAF_G469R | K562-19 | Mutant | 28z | 1.76072274 | 1.52697931 | 2.06857341 | 0.35364478 | NA |
| BRAF_G469R | K562-19 | Mutant | BBz | 1.46193293 | 1.21983288 | 1.96356853 | 0.00654301 | 0.09843618 |
| CARD11 | K562 | Wild-type | 28z | 0.76432902 | 1.40268456 | 0.57314851 | NA | NA |
| CARD11 | K562 | Wild-type | BBz | −1.03720751 | 0.42615385 | 0.01644312 | NA | −0.12678245 |
| CARD11 | K562-19 | Wild-type | 28z | 0.44778195 | 2.19188712 | 1.73088915 | 0.49655187 | NA |
| CARD11 | K562-19 | Wild-type | BBz | −0.34811726 | 1.52691894 | 0.37366488 | 0.04741407 | −0.17274699 |
| CARD11_D357N | K562 | Mutant | 28z | 0.81660002 | 4.77616697 | 1.31667239 | NA | NA |
| CARD11_D357N | K562 | Mutant | BBz | −0.86215615 | 4.04596443 | 0.38224895 | NA | −0.14513877 |
| CARD11_D357N | K562-19 | Mutant | 28z | 0.55371145 | 3.06936424 | 3.5186095 | 4.03791094 | NA |
| CARD11_D357N | K562-19 | Mutant | BBz | −0.23505497 | 3.32790316 | 2.25349661 | 3.3844891 | −0.41758481 |
| CARD11_E634K: | K562 | Mutant | 28z | 0.58427801 | 1.34030602 | 0.72247509 | NA | NA |
| S655C | | | | | | | | |
| CARD11_E634K: | K562 | Mutant | BBz | −1.04901776 | 0.57885195 | 0.22322985 | NA | −0.12951579 |
| S655C | | | | | | | | |
| CARD11_E634K: | K562-19 | Mutant | 28z | 0.14528109 | 2.45027526 | 2.26857329 | 0.69828143 | NA |
| S655C | | | | | | | | |
| CARD11_E634K: | K562-19 | Mutant | BBz | −0.37563674 | 2.4486072 | 1.07736493 | 0.68266188 | −0.27479898 |
| S655C | | | | | | | | |
| CARD11_S615F | K562 | Mutant | 28z | 0.31394837 | 1.90037921 | 0.75928438 | NA | NA |
| CARD11_S615F | K562 | Mutant | BBz | −0.77510162 | 0.58107106 | 0.06433776 | NA | −0.14552813 |
| CARD11_S615F | K562-19 | Mutant | 28z | 0.22611254 | 3.09528996 | 3.63508187 | 1.66924451 | NA |
| CARD11_S615F | K562-19 | Mutant | BBz | −0.07758086 | 3.21725938 | 2.61699929 | 1.62726411 | −0.34556963 |
| CARD11_Y361C | K562 | Mutant | 28z | 0.57506305 | 4.41637671 | 1.85631617 | NA | NA |
| CARD11_Y361C | K562 | Mutant | BBz | −1.00449277 | 2.71705204 | 0.2478598 | NA | −0.14162271 |
| CARD11_Y361C | K562-19 | Mutant | 28z | 0.32389953 | 3.168582 | 4.14743406 | 4.27085752 | NA |
| CARD11_Y361C | K562-19 | Mutant | BBz | −0.11740999 | 3.82923422 | 4.01651052 | 4.62183388 | −0.42416689 |
| CARD11-PIK3R3 | K562 | Fusion | 28z | 0.69354179 | 6.90840923 | 1.20497724 | NA | NA |
| CARD11-PIK3R3 | K562 | Fusion | BBz | 0.1466756 | 7.22743303 | 0.85507554 | NA | −0.14435763 |
| CARD11-PIK3R3 | K562-19 | Fusion | 28z | 1.51517617 | 3.13320621 | 0.3049936 | 4.66564406 | NA |
| CARD11-PIK3R3 | K562-19 | Fusion | BBz | 0.37107759 | 4.00945098 | 2.95110071 | 5.69050212 | −0.53650972 |
| CARMIL2_Q575E | K562 | Mutant | 28z | 1.72977003 | 0.06737486 | 0.21901141 | NA | NA |
| CARMIL2_Q575E | K562 | Mutant | BBz | −0.66414944 | −0.477902 | 0.53793429 | NA | −0.10647467 |
| CARMIL2_Q575E | K562-19 | Mutant | 28z | 0.63637423 | 0.02116175 | 0.51849677 | −0.25751396 | NA |
| CARMIL2_Q575E | K562-19 | Mutant | BBz | −0.44119459 | −0.26441597 | 0.06724462 | −0.21628463 | −0.04312157 |
| CCND3 | K562 | Wild-type | 28z | −0.95864479 | −0.32481792 | −1.14022793 | NA | NA |
| CCND3 | K562 | Wild-type | BBz | −2.00046112 | −0.62346197 | −1.19086847 | NA | −0.16232143 |
| CCND3 | K562-19 | Wild-type | 28z | −1.58580772 | −1.29423889 | −1.56376981 | −0.34298667 | NA |
| CCND3 | K562-19 | Wild-type | BBz | −1.12445669 | −1.02722958 | −1.69420663 | −0.24816655 | −0.4710794 |
| CCND3_P284S | K562 | Mutant | 28z | −1.09502855 | −0.41842547 | −1.30374901 | NA | NA |
| CCND3_P284S | K562 | Mutant | BBz | −2.50537585 | −0.75280415 | −1.24360496 | NA | −0.17013348 |
| CCND3_P284S | K562-19 | Mutant | 28z | −1.85171994 | −1.48521031 | −1.71008355 | −0.34298667 | NA |
| CCND3_P284S | K562-19 | Mutant | BBz | −1.65191799 | −1.4182088 | −1.76611827 | −0.26181593 | −0.55667426 |
| CD28 | K562 | Wild-type | 28z | −0.58014083 | −0.3764552 | −0.29434623 | NA | NA |
| CD28 | K562 | Wild-type | BBz | −0.47872898 | −0.45757179 | −0.2707104 | NA | −0.14592009 |
| CD28 | K562-19 | Wild-type | 28z | −0.05505631 | −0.45761568 | −0.16829841 | −0.26499215 | NA |
| CD28 | K562-19 | Wild-type | BBz | −0.5443677 | −0.69953906 | −0.52164624 | −0.25884193 | −0.04189137 |
| CD28_F51I | K562 | Mutant | 28z | −0.57621223 | −0.34975894 | −0.62237231 | NA | NA |
| CD28_F51I | K562 | Mutant | BBz | −0.72070639 | −0.3765616 | −0.1941139 | NA | −0.15529191 |
| CD28_F51I | K562-19 | Mutant | 28z | −0.15470396 | −0.65172335 | −0.49779072 | −0.29299669 | NA |
| CD28_F51I | K562-19 | Mutant | BBz | −0.55989238 | −0.62514142 | −0.59278357 | −0.22804159 | −0.07851296 |
| CD28_F51V | K562 | Mutant | 28z | −0.51884618 | −0.32456415 | −0.48693454 | NA | NA |
| CD28_F51V | K562 | Mutant | BBz | −0.36977225 | −0.42444839 | −0.38081091 | NA | −0.1498244 |
| CD28_F51V | K562-19 | Mutant | 28z | −0.23571422 | −0.73386965 | −0.37360963 | −0.30394058 | NA |
| CD28_F51V | K562-19 | Mutant | BBz | −0.48915964 | −0.65072433 | −0.31805897 | −0.24965553 | −0.030371 |
| CD28_Q77P | K562 | Mutant | 28z | −0.53216238 | −0.35701187 | −0.38039333 | NA | NA |
| CD28_Q77P | K562 | Mutant | BBz | −0.42416747 | −0.47278101 | −0.23361863 | NA | −0.15138761 |
| CD28_Q77P | K562-19 | Mutant | 28z | −0.24138729 | −0.48806784 | −0.35292875 | −0.23546789 | NA |
| CD28_Q77P | K562-19 | Mutant | BBz | −0.51274029 | −0.5997123 | −0.37119695 | −0.25269648 | 0.00707333 |
| CD28_T195P | K562 | Mutant | 28z | −0.44590801 | −0.34711941 | −0.37264683 | NA | NA |
| CD28_T195P | K562 | Mutant | BBz | −0.62145361 | −0.43013779 | −0.06551116 | NA | −0.13225238 |
| CD28_T195P | K562-19 | Mutant | 28z | 0.46152183 | −0.08448582 | 0.09520837 | −0.26199817 | NA |
| CD28_T195P | K562-19 | Mutant | BBz | −0.2143554 | −0.41628433 | 0.09413578 | −0.24803365 | 0.1375158 |
| CD28-CTLA4 | K562 | Fusion | 28z | −0.47332426 | −0.36779612 | −0.2118667 | NA | NA |
| CD28-CTLA4 | K562 | Fusion | BBz | 0.71391578 | −0.14055481 | −0.23057066 | NA | −0.14865473 |
| CD28-CTLA4 | K562-19 | Fusion | 28z | −0.3741551 | −0.40850329 | −0.64424609 | −0.26715955 | NA |
| CD28-CTLA4 | K562-19 | Fusion | BBz | −0.32034873 | −0.6219302 | −0.6635623 | −0.249844 | −0.08015496 |
| CD3E | K562 | Wild-type | 28z | −0.20869157 | −0.36252031 | −0.15002519 | NA | NA |
| CD3E | K562 | Wild-type | BBz | −0.77432561 | −0.63649336 | 0.10336625 | NA | −0.13186032 |
| CD3E | K562-19 | Wild-type | 28z | −0.07655176 | −0.33187641 | −0.12152951 | −0.30548728 | NA |
| CD3E | K562-19 | Wild-type | BBz | −0.20130779 | −0.30622441 | −0.03709553 | −0.23060918 | −0.04641549 |
| CD3E_S41C | K562 | Mutant | 28z | −0.22277297 | −0.36653084 | −0.44028483 | NA | NA |
| CD3E_S41C | K562 | Mutant | BBz | −0.84042023 | −0.59300352 | −0.18473182 | NA | −0.14318508 |
| CD3E_S41C | K562-19 | Mutant | 28z | −0.11814801 | −0.31809851 | −0.23428394 | −0.29767783 | NA |
| CD3E_S41C | K562-19 | Mutant | BBz | −0.28441598 | −0.51332993 | −0.30931003 | −0.21643876 | −0.17027997 |
| CD53 | K562 | Wild-type | 28z | −0.36542757 | −0.35020662 | −0.2877231 | NA | NA |
| CD53 | K562 | Wild-type | BBz | 1.03246777 | −0.14741347 | 0.20596587 | NA | −0.14748358 |
| CD53 | K562-19 | Wild-type | 28z | −0.15998843 | −0.22181842 | −0.46156325 | −0.27093551 | NA |
| CD53 | K562-19 | Wild-type | BBz | −0.29143329 | −0.48906853 | −0.28471326 | −0.22034481 | −0.15093587 |
| CSNK1A1 | K562 | Wild-type | 28z | −0.44874715 | −0.33704702 | −0.42349776 | NA | NA |
| CSNK1A1 | K562 | Wild-type | BBz | −0.92780734 | −0.48795022 | −0.33677387 | NA | −0.15997838 |
| CSNK1A1 | K562-19 | Wild-type | 28z | −0.35891914 | −0.57390489 | −0.43016389 | −0.28566222 | NA |
| CSNK1A1 | K562-19 | Wild-type | BBz | −0.38452477 | −0.27172985 | −0.44943316 | −0.22846393 | −0.1521738 |
| CSNK1A1_S27C | K562 | Mutant | 28z | −0.31294661 | −0.30901164 | −0.52438639 | NA | NA |
| CSNK1A1_S27C | K562 | Mutant | BBz | −0.56733553 | −0.27275759 | −0.35643497 | NA | −0.14123307 |
| CSNK1A1_S27C | K562-19 | Mutant | 28z | −0.20514256 | −0.34914646 | −0.37183918 | −0.26088941 | NA |
| CSNK1A1_S27C | K562-19 | Mutant | BBz | −0.30094182 | −0.19923758 | −0.18489185 | −0.24110229 | −0.09909219 |
| CSNK2B | K562 | Wild-type | 28z | −0.51786169 | −0.24503595 | −0.56053973 | NA | NA |
| CSNK2B | K562 | Wild-type | BBz | −0.50168434 | −0.04736048 | −0.67212366 | NA | −0.14709013 |
| CSNK2B | K562-19 | Wild-type | 28z | −0.3803699 | −0.40786621 | −0.49920708 | −0.24916952 | NA |
| CSNK2B | K562-19 | Wild-type | BBz | −0.43728406 | −0.27377335 | −0.55126321 | −0.23935517 | −0.00568437 |
| CSNK2B_Q182X | K562 | Mutant | 28z | −0.67970208 | −0.34188232 | −0.62332948 | NA | NA |
| CSNK2B_Q182X | K562 | Mutant | BBz | −0.98270142 | −0.35146927 | −0.75707037 | NA | −0.150606 |
| CSNK2B_Q182X | K562-19 | Mutant | 28z | −0.3451449 | −0.65386662 | −0.61408605 | −0.32332219 | NA |
| CSNK2B_Q182X | K562-19 | Mutant | BBz | −0.39709762 | −0.51274607 | −0.76661099 | −0.23277307 | −0.19291069 |
| DGKA | K562 | Wild-type | 28z | −0.39000468 | −0.36890619 | −0.15889508 | NA | NA |
| DGKA | K562 | Wild-type | BBz | 0.30986127 | −0.26049532 | 0.22635721 | NA | −0.14748237 |
| DGKA | K562-19 | Wild-type | 28z | −0.34400475 | −0.32754857 | −0.27622474 | −0.32293663 | NA |
| DGKA | K562-19 | Wild-type | BBz | −0.19168181 | −0.45998248 | −0.27295276 | −0.24893913 | −0.11761202 |
| DGKZ | K562 | Wild-type | 28z | −0.55927303 | −0.40663331 | −0.35859179 | NA | NA |
| DGKZ | K562 | Wild-type | BBz | 0.11179913 | −0.23052019 | 0.24430382 | NA | −0.14982672 |
| DGKZ | K562-19 | Wild-type | 28z | −1.95081386 | −2.01088233 | −1.60264003 | −0.34298667 | NA |
| DGKZ | K562-19 | Wild-type | BBz | −1.19877096 | −2.04362801 | −1.53464225 | −0.25612365 | −0.4965937 |
| ECSIT | K562 | Wild-type | 28z | −0.23687303 | −0.24493021 | −0.46175394 | NA | NA |
| ECSIT | K562 | Wild-type | BBz | −0.72081725 | −0.20094585 | −0.52462183 | NA | −0.13849964 |
| ECSIT | K562-19 | Wild-type | 28z | −0.39929618 | −0.34501722 | −0.42268011 | −0.30511933 | NA |
| ECSIT | K562-19 | Wild-type | BBz | −0.50135184 | −0.28970695 | −0.63286958 | −0.23625678 | −0.04642178 |
| ECSIT_V140A | K562 | Mutant | 28z | −0.64210503 | −0.33154217 | −0.58523506 | NA | NA |
| ECSIT_V140A | K562 | Mutant | BBZ | −0.70911786 | −0.30636496 | −0.4532958 | NA | −0.15099537 |
| ECSIT_V140A | K562-19 | Mutant | 28z | −0.46486066 | −0.52145765 | −0.55558429 | −0.26915792 | NA |
| ECSIT_V140A | K562-19 | Mutant | BBz | −0.52833077 | −0.43089642 | −0.52594927 | −0.25689741 | −0.12295566 |
| EIFS1 | K562 | Wild-type | 28z | −0.33400571 | −0.34881446 | 0.0336754 | NA | NA |
| EIFS1 | K562 | Wild-type | BBz | −0.90712457 | −0.55524309 | 0.11098623 | NA | −0.14826305 |
| EIFS1 | K562-19 | Wild-type | 28z | −0.24866653 | −0.34009359 | −0.12706579 | −0.30538397 | NA |
| EIFS1 | K562-19 | Wild-type | BBz | −0.1668651 | 0.03097729 | 0.11486094 | −0.22898211 | −0.1920877 |
| EIFS1_R89I | K562 | Mutant | 28z | −0.43376759 | −0.27968626 | −0.47256045 | NA | NA |
| EIFS1_R89I | K562 | Mutant | BBz | −0.8080935 | −0.33537153 | −0.38488282 | NA | −0.15490199 |
| EIFS1_R89I | K562-19 | Mutant | 28z | −0.40268728 | −0.4565814 | −0.47281287 | −0.20454285 | NA |
| EIFS1_R89I | K562-19 | Mutant | BBz | −0.44297121 | −0.22071782 | −0.29790811 | −0.23771353 | −0.18385599 |
| FYN | K562 | Wild-type | 28z | −0.05799779 | −0.20253699 | −0.00215442 | NA | NA |
| FYN | K562 | Wild-type | BBz | −0.01905981 | −0.31568471 | −0.15599027 | NA | −0.11975312 |
| FYN | K562-19 | Wild-type | 28z | −0.85921924 | −1.1977602 | −0.92289747 | −0.34298667 | NA |
| FYN | K562-19 | Wild-type | BBZ | −0.3281698 | −0.00308721 | 0.08058235 | −0.25575489 | −0.25875356 |
| FYN_R86G | K562 | Mutant | 28z | 0.75297246 | 0.21942135 | 0.80158894 | NA | NA |
| FYN_R86G | K562 | Mutant | BBz | −0.07484076 | −0.47378958 | 0.08973765 | NA | −0.12561306 |
| FYN_R86G | K562-19 | Mutant | 28z | −0.04036374 | −0.62345576 | −0.79588366 | −0.34164038 | NA |
| FYN_R86G | K562-19 | Mutant | BBz | 0.18905917 | 0.31539973 | 0.48388528 | −0.18699662 | −0.22665185 |
| FYN_Y531H | K562 | Mutant | 28z | −0.14062917 | −0.1530213 | 0.05741357 | NA | NA |
| FYN_Y531H | K562 | Mutant | BBz | −0.07367675 | −0.3354128 | −0.23575382 | NA | −0.12678393 |
| FYN_Y531H | K562-19 | Mutant | 28z | −0.46888288 | −0.96120984 | −0.85903648 | −0.34287395 | NA |
| FYN_Y531H | K562-19 | Mutant | BBz | 0.0378552 | 0.28382445 | 0.10854927 | −0.2305466 | −0.28673403 |
| GATA3 | K562 | Wild-type | 28z | −0.25312402 | −0.10426333 | −0.04252235 | NA | NA |
| GATA3 | K562 | Wild-type | BBz | 0.42846653 | −0.29107382 | −0.06983707 | NA | −0.12639411 |
| GATA3 | K562-19 | Wild-type | 28z | 0.52722908 | 0.29999748 | −0.07303806 | −0.24319387 | NA |
| GATA3 | K562-19 | Wild-type | BBz | 0.03539228 | −0.02472126 | −0.29166927 | −0.21948347 | −0.3636831 |
| GATA3_Y63X | K562 | Mutant | 28z | −0.65513429 | −0.30733058 | −0.40944273 | NA | NA |
| GATA3_Y63X | K562 | Mutant | BBz | −1.07125254 | −0.37426247 | −0.91994707 | NA | −0.14904382 |
| GATA3_Y63X | K562-19 | Mutant | 28z | −0.27944967 | −0.43875685 | −0.56807322 | −0.24868349 | NA |
| GATA3_Y63X | K562-19 | Mutant | BBz | −0.44090309 | −0.52955546 | −0.93240665 | −0.23567896 | −0.11513939 |
| GNAQ | K562 | Wild-type | 28z | −0.44688316 | −0.35104536 | −0.33518606 | NA | NA |
| GNAQ | K562 | Wild-type | BBZ | −0.15075019 | −0.25073099 | −0.28754576 | NA | −0.15411937 |
| GNAQ | K562-19 | Wild-type | 28z | −0.23181408 | −0.29396016 | −0.39681704 | −0.29613938 | NA |
| GNAQ | K562-19 | Wild-type | BBz | −0.39717757 | −0.35328757 | −0.41451023 | −0.20870113 | −0.14517238 |
| GNAQ_T96S | K562 | Mutant | 28z | −0.4353353 | −0.32822234 | −0.33067469 | NA | NA |
| GNAQ_T96S | K562 | Mutant | BBz | −0.89625661 | −0.3048718 | 0.04248594 | NA | −0.13889039 |
| GNAQ_T96S | K562-19 | Mutant | 28z | −0.3181088 | −0.18469656 | −0.18184961 | −0.31291166 | NA |
| GNAQ_T96S | K562-19 | Mutant | BBz | −0.4363487 | −0.20902502 | −0.0973324 | −0.22998832 | −0.04394915 |
| ICOS-CD28 | K562 | Fusion | 28z | −0.51884618 | −0.38236185 | −0.26868927 | NA | NA |
| ICOS-CD28 | K562 | Fusion | BBz | 0.6594097 | −0.11098172 | 0.05786873 | NA | −0.14006563 |
| ICOS-CD28 | K562-19 | Fusion | 28z | 0.03735682 | −0.4839386 | −0.45996985 | −0.3169841 | NA |
| ICOS-CD28 | K562-19 | Fusion | BBz | −0.32080934 | −0.54095634 | −0.27775725 | −0.26181593 | −0.0118555 |
| IRF4 | K562 | Wild-type | 28z | −0.80277896 | −0.30963891 | −0.28951135 | NA | NA |
| IRF4 | K562 | Wild-type | BBz | −1.333691 | −0.51429436 | −0.14121072 | NA | −0.15763533 |
| IRF4 | K562-19 | Wild-type | 28z | −1.37738118 | −0.05069881 | 0.03311782 | −0.32231906 | NA |
| IRF4 | K562-19 | Wild-type | BBz | −1.44081503 | −0.823781 | −0.50723276 | −0.26181593 | 0.53667093 |
| IRF4_K59R | K562 | Mutant | 28z | −0.9342683 | −0.22734071 | 0.67516944 | NA | NA |
| IRF4_K59R | K562 | Mutant | BBz | −1.73863237 | −0.40137383 | 0.19384536 | NA | −0.14709078 |
| IRF4_K59R | K562-19 | Mutant | 28z | −1.3313861 | 0.75404077 | 1.27079425 | −0.34156794 | NA |
| IRF4_K59R | K562-19 | Mutant | BBZ | −1.48668861 | −0.4887766 | −0.19041351 | −0.26181593 | 0.26097145 |
| ITGB2 | K562 | Wild-type | 28z | −0.02169084 | −0.34878973 | −0.00313834 | NA | NA |
| ITGB2 | K562 | Wild-type | BBZ | −0.60005026 | −0.64766956 | 0.20716443 | NA | −0.11545778 |
| ITGB2 | K562-19 | Wild-type | 28z | −0.19610073 | −0.3393405 | −0.1822037 | −0.29301631 | NA |
| ITGB2 | K562-19 | Wild-type | BBz | −0.10175371 | −0.3230338 | −0.03494402 | −0.23935517 | −0.11225607 |
| ITGB2_E234K | K562 | Mutant | 28z | −0.15081928 | −0.26040171 | −0.16747267 | NA | NA |
| ITGB2_E234K | K562 | Mutant | BBz | −0.31537702 | −0.52119303 | −0.06812259 | NA | −0.11740942 |
| ITGB2_E234K | K562-19 | Mutant | 28z | 0.0755843 | −0.2849073 | −0.26479808 | −0.24113582 | NA |
| ITGB2_E234K | K562-19 | Mutant | BBz | −0.33917759 | −0.6567167 | −0.21644462 | −0.25459582 | −0.15834883 |
| ITK-FER | K562 | Fusion | 28z | 0.30650175 | −0.26552591 | 0.339244 | NA | NA |
| ITK-FER | K562 | Fusion | BBZ | 0.62763726 | −0.30559963 | 0.56295316 | NA | −0.15841731 |
| ITK-FER | K562-19 | Fusion | 28z | 0.25918967 | −0.1613113 | −0.08105297 | −0.25876702 | NA |
| ITK-FER | K562-19 | Fusion | BBZ | 0.3190046 | −0.0643324 | 0.13673189 | −0.23981607 | −0.10443676 |
| ITK-SYK | K562 | Fusion | 28z | −0.46775096 | −0.34064177 | −0.13857165 | NA | NA |
| ITK-SYK | K562 | Fusion | BBz | 0.72555975 | −0.09117297 | 0.05354281 | NA | −0.15138751 |
| ITK-SYK | K562-19 | Fusion | 28z | −0.16785575 | −0.51165169 | −0.68242105 | −0.28232082 | NA |
| ITK-SYK | K562-19 | Fusion | BBz | −0.36070427 | −0.49155775 | −0.56230655 | −0.25434095 | −0.13118414 |
| JAK1_G1097A | K562 | Mutant | 28z | 0.42310718 | −0.20649125 | 0.30278053 | NA | NA |
| JAK1_G1097A | K562 | Mutant | BBz | −0.95031926 | −0.61664585 | 0.231969 | NA | −0.11936376 |
| JAK1_G1097A | K562-19 | Mutant | 28z | 0.34262633 | 0.01806679 | 0.50105059 | −0.29272241 | NA |
| JAK1_G1097A | K562-19 | Mutant | BBz | −0.0592784 | −0.07528756 | 0.14885098 | −0.23495171 | −0.03242997 |
| JAK3 | K562 | Wild-type | 28z | −0.25558056 | −0.2954291 | −0.1403376 | NA | NA |
| JAK3 | K562 | Wild-type | BBz | −0.45715935 | −0.5203064 | 0.06271058 | NA | −0.13654595 |
| JAK3 | K562-19 | Wild-type | 28z | −0.06437501 | −0.30261584 | −0.32223756 | −0.29898304 | NA |
| JAK3 | K562-19 | Wild-type | BBZ | −0.33314729 | −0.36073958 | −0.2982667 | −0.22208007 | −0.17727392 |
| JAK3_A573V | K562 | Mutant | 28z | −0.22464628 | −0.33008657 | −0.15794237 | NA | NA |
| JAK3_A573V | K562 | Mutant | BBz | −0.4910381 | −0.54463151 | 0.2654254 | NA | −0.11701858 |
| JAK3_A573V | K562-19 | Mutant | 28z | −0.04707723 | −0.00158642 | 0.10858252 | −0.26096146 | NA |
| JAK3_A573V | K562-19 | Mutant | BBz | −0.14718084 | −0.31542799 | 0.02177982 | −0.23573164 | −0.06699879 |
| JUNB | K562 | Wild-type | 28z | −0.44669186 | −0.33746281 | −0.62478529 | NA | NA |
| JUNB | K562 | Wild-type | BBz | −0.54504532 | −0.43211554 | −0.71144585 | NA | −0.16856981 |
| JUNB | K562-19 | Wild-type | 28z | 0.94164374 | −0.28510589 | −0.82050743 | −0.23656332 | NA |
| JUNB | K562-19 | Wild-type | BBz | −0.18029336 | −0.63961538 | −1.46348391 | −0.23480652 | −0.33817152 |
| JUNB_A282V | K562 | Mutant | 28z | −0.69517854 | −0.35490786 | −0.76877835 | NA | NA |
| JUNB_A282V | K562 | Mutant | BBz | −0.74210974 | −0.26477095 | −0.63257923 | NA | −0.16232162 |
| JUNB_A282V | K562-19 | Mutant | 28z | −0.17477039 | −0.44149591 | −1.51788904 | −0.33009102 | NA |
| JUNB_A282V | K562-19 | Mutant | BBz | −0.49771866 | −0.27830607 | −1.70377976 | −0.25632839 | −0.33775973 |
| KCNQ1 | K562 | Wild-type | 28z | −0.57289484 | −0.35789649 | −0.56554082 | NA | NA |
| KCNQ1 | K562 | Wild-type | BBz | −0.42433376 | −0.39318396 | −0.31942259 | NA | −0.16114991 |
| KCNQ1 | K562-19 | Wild-type | 28z | −0.18270476 | −0.32187185 | −0.53166871 | −0.19437329 | NA |
| KCNQ1 | K562-19 | Wild-type | BBz | −0.29417356 | −0.57486705 | −0.58833766 | −0.23668669 | −0.2603987 |
| KCNQ1_R583C | K562 | Mutant | 28z | −0.54665436 | −0.35848146 | −0.44108024 | NA | NA |
| KCNQ1_R583C | K562 | Mutant | BBz | −0.66620031 | −0.40875584 | −0.35545072 | NA | −0.16622722 |
| KCNQ1_R583C | K562-19 | Mutant | 28z | −0.35436554 | −0.35407009 | −0.48565588 | −0.24148623 | NA |
| KCNQ1_R583C | K562-19 | Mutant | BBz | −0.32406186 | −0.48074073 | −0.46693104 | −0.23464042 | −0.19908496 |
| MSC | K562 | Wild-type | 28z | −0.72181095 | −0.39528524 | −0.83416068 | NA | NA |
| MSC | K562 | Wild-type | BBz | −1.14727283 | −0.23939536 | −0.83007119 | NA | −0.15919771 |
| MSC | K562-19 | Wild-type | 28z | −1.13520336 | −1.39933196 | −1.06386155 | −0.34164038 | NA |
| MSC | K562-19 | Wild-type | BBz | −0.93974045 | −1.53484494 | −1.06220362 | −0.26166008 | 0.31445534 |
| MSC_E116K | K562 | Mutant | 28z | −0.66286786 | −0.10499992 | −0.4270475 | NA | NA |
| MSC_E116K | K562 | Mutant | BBz | −1.1472174 | 0.76604342 | −0.67473508 | NA | −0.16349332 |
| MSC_E116K | K562-19 | Mutant | 28z | −1.26808291 | −0.07448126 | −0.22644608 | −0.30387608 | NA |
| MSC_E116K | K562-19 | Mutant | BBz | −1.35047797 | −0.12937266 | −1.09519074 | −0.23662239 | −0.39577403 |
| MYCN | K562 | Wild-type | 28z | −0.53441829 | −0.36907896 | −0.73052664 | NA | NA |
| MYCN | K562 | Wild-type | BBz | −0.52375283 | −0.13385621 | −0.61985549 | NA | −0.15490153 |
| MYCN | K562-19 | Wild-type | 28z | −1.29255851 | −1.00813766 | −1.02119153 | −0.291337 | NA |
| MYCN | K562-19 | Wild-type | BBz | −0.84821898 | −0.72716547 | −1.1055169 | −0.23523482 | −0.23694413 |
| MYCN_P44L | K562 | Mutant | 28z | −0.71896248 | −0.37573976 | −0.89470367 | NA | NA |
| MYCN_P44L | K562 | Mutant | BBz | −1.26831696 | −0.09690238 | −0.74096526 | NA | −0.16115074 |
| MYCN_P44L | K562-19 | Mutant | 28z | −1.35072167 | −1.23801831 | −1.21374971 | −0.29281915 | NA |
| MYCN_P44L | K562-19 | Mutant | BBz | −0.98499353 | −0.77057168 | −1.19572933 | −0.2517801 | −0.29414444 |
| NFKB1 | K562 | Wild-type | 28z | 0.08092883 | −0.01440897 | 0.16505587 | NA | NA |
| NFKB1 | K562 | Wild-type | BBz | 0.00262068 | 0.14160741 | 0.53206058 | NA | −0.14982551 |
| NFKB1 | K562-19 | Wild-type | 28z | 0.53398727 | 0.58764619 | 0.21810224 | −0.28627586 | NA |
| NFKB1 | K562-19 | Wild-type | BBz | 0.39541034 | 0.39096508 | 0.32482993 | −0.20340792 | −0.14681573 |
| NFKB1_H67Y | K562 | Mutant | 28z | 0.9437595 | −0.15284853 | 0.70554411 | NA | NA |
| NFKB1_H67Y | K562 | Mutant | BBz | −0.17276325 | −0.53781539 | 0.5342513 | NA | −0.12639411 |
| NFKB1_H67Y | K562-19 | Mutant | 28z | 0.17675038 | 0.40592624 | 0.66606231 | −0.26262686 | NA |
| NFKB1_H67Y | K562-19 | Mutant | BBz | 0.5531618 | −0.06316469 | 0.80722912 | −0.16511798 | −0.24351568 |
| NFKB2 | K562 | Wild-type | 28z | 0.01532298 | −0.41819285 | −0.1576144 | NA | NA |
| NFKB2 | K562 | Wild-type | BBz | 0.03594513 | −0.69011208 | 0.23364379 | NA | −0.11975423 |
| NFKB2 | K562-19 | Wild-type | 28z | −0.06337762 | −1.17143728 | 0.01956663 | −0.29264744 | NA |
| NFKB2 | K562-19 | Wild-type | BBz | 0.16688863 | −1.31005425 | 0.19252285 | −0.14371182 | −0.09538002 |
| NFKB2_K656X | K562 | Mutant | 28z | −0.28259556 | −0.0751247 | −0.36198869 | NA | NA |
| NFKB2_K656X | K562 | Mutant | BBz | 0.45197618 | 0.51393383 | −0.1155886 | NA | −0.16076017 |
| NFKB2_K656X | K562-19 | Mutant | 28z | −0.07854653 | −0.56524921 | −0.53738204 | −0.25383021 | NA |
| NFKB2_K656X | K562-19 | Mutant | BBz | −0.25300012 | −0.54124827 | −0.52824367 | −0.17765405 | −0.25998173 |
| NPM-ALK | K562 | Fusion | 28z | −0.29188751 | −0.24869056 | 0.82144494 | NA | NA |
| NPM-ALK | K562 | Fusion | BBz | 0.71469179 | −0.25182085 | 1.16339034 | NA | −0.11975637 |
| NPM-ALK | K562-19 | Fusion | 28z | −0.25193555 | −0.47250257 | 1.0881593 | −0.33706929 | NA |
| NPM-ALK | K562-19 | Fusion | BBz | −0.19015425 | −0.19324521 | 0.60686912 | −0.22966099 | −0.34680161 |
| NPM-TYK | K562 | Fusion | 28z | 0.03245347 | −0.32985395 | 0.60945468 | NA | NA |
| NPM-TYK | K562 | Fusion | BBz | 0.37423759 | −0.22600195 | 0.46904507 | NA | −0.13498571 |
| NPM-TYK | K562-19 | Fusion | 28z | 0.53919769 | −0.2843115 | 0.60809169 | −0.34123768 | NA |
| NPM-TYK | K562-19 | Fusion | BBz | 0.5999941 | −0.01448809 | 0.92705852 | −0.2552337 | −0.1826134 |
| NRAS | K562 | Wild-type | 28z | −0.26714708 | −0.3438383 | −0.23332688 | NA | NA |
| NRAS | K562 | Wild-type | BBz | 0.49650117 | −0.05882057 | 0.05050276 | NA | −0.16661918 |
| NRAS | K562-19 | Wild-type | 28z | −0.29809741 | −0.13872043 | −0.06267367 | −0.27460561 | NA |
| NRAS | K562-19 | Wild-type | BBz | −0.12336034 | −0.1656188 | 0.02084694 | −0.21096022 | −0.2089562 |
| NRAS_G13D | K562 | Mutant | 28z | −0.44424465 | −0.37768858 | −0.20491114 | NA | NA |
| NRAS_G13D | K562 | Mutant | BBz | 0.09045122 | 0.11465611 | 0.29224607 | NA | −0.14240645 |
| NRAS_G13D | K562-19 | Mutant | 28z | −0.88362946 | −0.61201973 | −0.44925137 | −0.27116567 | NA |
| NRAS_G13D | K562-19 | Mutant | BBz | −0.40551089 | −0.51157836 | −0.21164013 | −0.20718884 | −0.26779935 |
| NRAS_Q61H | K562 | Mutant | 28z | −0.47321929 | −0.40909683 | −0.44480065 | NA | NA |
| NRAS_Q61H | K562 | Mutant | BBz | 0.71552322 | −0.05664085 | −0.12450236 | NA | −0.14631215 |
| NRAS_Q61H | K562-19 | Mutant | 28z | −1.07258633 | −0.77392917 | −0.69597225 | −0.32639748 | NA |
| NRAS_Q61H | K562-19 | Mutant | BBz | −0.46875157 | −0.55776573 | −0.47173554 | −0.21659873 | −0.30071955 |
| PDCD1 | K562 | Wild-type | 28z | −0.02032376 | −0.36001807 | −0.07108648 | NA | NA |
| PDCD1 | K562 | Wild-type | BBz | −0.30395477 | −0.30850404 | −0.70926304 | NA | 7.59073915 |
| PDCD1 | K562-19 | Wild-type | 28z | −0.05106677 | −0.28077806 | −0.09978636 | −0.30768851 | NA |
| PDCD1 | K562-19 | Wild-type | BBz | −0.04306742 | −0.42768521 | −0.89246352 | −0.249844 | 7.43334981 |
| PDCD1_R231X | K562 | Mutant | 28z | −0.24256996 | −0.35055896 | −0.39878905 | NA | NA |
| PDCD1_R231X | K562 | Mutant | BBz | 0.04648052 | −0.13083113 | −1.02023688 | NA | 7.55949913 |
| PDCD1_R231X | K562-19 | Mutant | 28z | −0.35566046 | −0.33910061 | −0.29796789 | −0.24374415 | NA |
| PDCD1_R231X | K562-19 | Mutant | BBz | −0.73519419 | −0.80156311 | −0.65710776 | −0.24165014 | 7.41689166 |
| PLCG1 | K562 | Wild-type | 28z | −0.14592485 | −0.27006513 | −0.27255806 | NA | NA |
| PLCG1 | K562 | Wild-type | BBz | −0.0511094 | −0.13591652 | 0.06682219 | NA | −0.12717747 |
| PLCG1 | K562-19 | Wild-type | 28z | −0.10502891 | −0.18763421 | −0.30261895 | 0.03183223 | NA |
| PLCG1 | K562-19 | Wild-type | BBz | 0.14712442 | −0.17993896 | −0.17291563 | −0.25344572 | −0.20360229 |
| PLCG1_D1165H | K562 | Mutant | 28z | 5.0043194 | 1.06939699 | 1.74380332 | NA | NA |
| PLCG1_D1165H | K562 | Mutant | BBz | 0.96482113 | −0.12252247 | 1.14691213 | NA | −0.07289151 |
| PLCG1_D1165H | K562-19 | Mutant | 28z | 1.99928762 | 1.71191811 | 1.51415128 | 0.16042837 | NA |
| PLCG1_D1165H | K562-19 | Mutant | BBz | 3.39863359 | 1.52384586 | 2.50979045 | −0.1017419 | 0.02518722 |
| PLCG1_E1163K | K562 | Mutant | 28z | 0.95874839 | 0.32092343 | 0.57719243 | NA | NA |
| PLCG1_E1163K | K562 | Mutant | BBZ | 0.27698025 | −0.34953342 | 0.51171685 | NA | −0.13420541 |
| PLCG1_E1163K | K562-19 | Mutant | 28z | 1.78122081 | 1.6305662 | 1.08474755 | 0.83569131 | NA |
| PLCG1_E1163K | K562-19 | Mutant | BBZ | 2.24403946 | 1.16736121 | 1.47865923 | 0.23789494 | −0.03118194 |
| PLCG1_E47K | K562 | Mutant | 28z | 0.42241897 | −0.02241964 | 0.10582477 | NA | NA |
| PLCG1_E47K | K562 | Mutant | BBz | 0.17678517 | −0.23237601 | −0.10701616 | NA | −0.14552971 |
| PLCG1_E47K | K562-19 | Mutant | 28z | 1.30608581 | 1.1979222 | 0.44967853 | 0.54289919 | NA |
| PLCG1_E47K | K562-19 | Mutant | BBz | 1.09976681 | 0.61371227 | 0.43268311 | 0.11867278 | 0.06017267 |
| PLCG1_R48W | K562 | Mutant | 28z | 1.22927865 | 0.56336014 | 0.22803413 | NA | NA |
| PLCG1_R48W | K562 | Mutant | BBz | 0.92157101 | −0.20925892 | −0.14827507 | NA | −0.11819362 |
| PLCG1_R48W | K562-19 | Mutant | 28z | 2.42644478 | 1.78223119 | 0.94506778 | 2.3504768 | NA |
| PLCG1_R48W | K562-19 | Mutant | BBz | 2.908172 | 1.57544174 | 1.95503529 | 1.28637241 | 0.46261435 |
| PLCG1_S520F | K562 | Mutant | 28z | 4.00894029 | 0.35879209 | 0.41078202 | NA | NA |
| PLCG1_S520F | K562 | Mutant | BBz | 1.75612741 | 0.18380354 | 0.56354848 | NA | −0.0775803 |
| PLCG1_S520F | K562-19 | Mutant | 28z | 1.61058144 | 1.19299858 | 1.21818287 | −0.08617912 | NA |
| PLCG1_S520F | K562-19 | Mutant | BBz | 1.95371054 | 0.74087351 | 1.26861741 | −0.1929633 | −0.01965869 |
| PRKCB1 | K562 | Wild-type | 28z | 0.04214668 | −0.31159131 | −0.04847615 | NA | NA |
| PRKCB1 | K562 | Wild-type | BBz | 0.1781709 | −0.22737381 | 0.30788287 | NA | −0.15021608 |
| PRKCB1 | K562-19 | Wild-type | 28z | −0.25562587 | 0.20157412 | 0.25791851 | −0.24507385 | NA |
| PRKCB1 | K562-19 | Wild-type | BBz | 0.11061361 | 0.30283114 | 0.20944644 | −0.23705736 | −0.27273543 |
| PRKCB1_D427N | K562 | Mutant | 28z | −0.31794602 | −0.15959751 | 0.00657155 | NA | NA |
| PRKCB1_D427N | K562 | Mutant | BBz | 0.48496806 | −0.12986446 | −0.03132451 | NA | −0.14631057 |
| PRKCB1_D427N | K562-19 | Mutant | 28z | −1.02109531 | 0.57795624 | −0.47941142 | −0.20952336 | NA |
| PRKCB1_D427N | K562-19 | Mutant | BBz | −0.94284723 | 0.58316658 | −0.53218811 | −0.24893913 | −0.42704698 |
| PRKCB1_D630Y | K562 | Mutant | 28z | −0.1190055 | −0.29334981 | −0.1422876 | NA | NA |
| PRKCB1_D630Y | K562 | Mutant | BBz | −0.28188628 | −0.26412693 | 0.19992547 | NA | −0.14709366 |
| PRKCB1_D630Y | K562-19 | Mutant | 28z | −0.61693134 | 0.16072021 | −0.04535664 | −0.30064774 | NA |
| PRKCB1_D630Y | K562-19 | Mutant | BBz | −0.27342234 | 0.33878534 | −0.11196159 | −0.2480327 | −0.265738 |
| RARA | K562 | Wild-type | 28z | 0.07357786 | −0.39766059 | −0.16504631 | NA | NA |
| RARA | K562 | Wild-type | BBz | 2.33556585 | −0.25638037 | 0.10486643 | NA | −0.14201467 |
| RARA | K562-19 | Wild-type | 28z | 0.97211566 | −0.75526894 | −0.15952742 | −0.33095303 | NA |
| RARA | K562-19 | Wild-type | BBz | 1.18566693 | −0.39465029 | −0.09539659 | −0.2605096 | 0.35151419 |
| RARA_G206S | K562 | Mutant | 28z | 0.15758565 | −0.40171701 | −0.10434602 | NA | NA |
| RARA_G206S | K562 | Mutant | BBz | 2.76202142 | −0.15996026 | 0.38169332 | NA | −0.14201504 |
| RARA_G206S | K562-19 | Mutant | 28z | 0.8819208 | −0.65958464 | −0.17755264 | −0.34298667 | NA |
| RARA_G206S | K562-19 | Mutant | BBz | 1.34330094 | −0.31338449 | 0.03224886 | −0.26181593 | 0.52022127 |
| RASGRP1 | K562 | Wild-type | 28z | 2.57715222 | 0.35289874 | 1.81260038 | NA | NA |
| RASGRP1 | K562 | Wild-type | BBz | 1.22692702 | −0.03808012 | 1.06901393 | NA | −0.09085726 |
| RASGRP1 | K562-19 | Wild-type | 28z | 2.61980049 | 1.72855108 | 1.5701744 | 0.302134 | NA |
| RASGRP1 | K562-19 | Wild-type | BBz | 2.80678965 | 1.4525213 | 2.32405964 | −0.02974785 | −0.12870888 |
| RASGRP1_M261I | K562 | Mutant | 28z | 2.45896976 | 0.80454914 | 2.99486157 | NA | NA |
| RASGRP1_M261I | K562 | Mutant | BBz | 2.04185381 | 0.35441582 | 1.86018033 | NA | −0.02485699 |
| RASGRP1_M261I | K562-19 | Mutant | 28z | 3.24928982 | 1.85592042 | 2.35935963 | 2.81543692 | NA |
| RASGRP1_M261I | K562-19 | Mutant | BBz | 4.01905476 | 1.79848086 | 2.80839464 | 0.57657597 | −0.1377674 |
| RHOA | K562 | Wild-type | 28z | −0.36170892 | −0.34721799 | −0.60897323 | NA | NA |
| RHOA | K562 | Wild-type | BBz | 0.63839436 | −0.2408085 | −0.27940985 | NA | −0.15255839 |
| RHOA | K562-19 | Wild-type | 28z | −0.23892652 | −0.40592153 | −0.53738204 | −0.32101367 | NA |
| RHOA | K562-19 | Wild-type | BBz | −0.49691491 | −0.48074073 | −0.59421791 | −0.26181593 | −0.24804846 |
| RHOA_C16R | K562 | Mutant | 28z | −0.58532221 | −0.3704851 | −0.5324475 | NA | NA |
| RHOA_C16R | K562 | Mutant | BBz | 0.28845793 | −0.07225337 | −0.45950291 | NA | −0.17208688 |
| RHOA_C16R | K562-19 | Mutant | 28z | −0.3381322 | −0.39317792 | −0.60018076 | −0.28372207 | NA |
| RHOA_C16R | K562-19 | Mutant | BBz | −0.23165669 | −0.34334633 | −0.56639387 | −0.25164868 | −0.28508287 |
| RHOA_G17V | K562 | Mutant | 28z | −0.40410474 | −0.33326553 | −0.56151919 | NA | NA |
| RHOA_G17V | K562 | Mutant | BBZ | 0.39591808 | −0.05345318 | −0.17230969 | NA | −0.17052395 |
| RHOA_G17V | K562-19 | Mutant | 28z | −0.21093773 | −0.29630203 | −0.46709954 | −0.28010727 | NA |
| RHOA_G17V | K562-19 | Mutant | BBz | −0.18783708 | −0.28371458 | −0.30966861 | −0.249844 | −0.2077192 |
| SELENOI-ABL1 | K562 | Fusion | 28z | −0.22444566 | 0.69022259 | 0.11938116 | NA | NA |
| SELENOI-ABL1 | K562 | Fusion | BBz | −0.10689035 | 0.59579253 | 0.24426412 | NA | −0.14435902 |
| SELENOI-ABL1 | K562-19 | Fusion | 28z | 1.09377981 | 2.13372187 | 0.41757099 | −0.313734 | NA |
| SELENOI-ABL1 | K562-19 | Fusion | BBz | 0.91589183 | 1.49637324 | 0.42809432 | −0.232441 | −0.36655742 |
| SMARCB1 | K562 | Wild-type | 28z | −0.50171568 | −0.37922877 | −0.54617456 | NA | NA |
| SMARCB1 | K562 | Wild-type | BBz | −0.10711207 | −0.0955718 | −0.33591663 | NA | −0.16154085 |
| SMARCB1 | K562-19 | Wild-type | 28z | −0.47709412 | −0.61833354 | −0.57484882 | −0.3284938 | NA |
| SMARCB1 | K562-19 | Wild-type | BBz | −0.12126392 | −0.49360125 | −0.54875311 | −0.26181593 | −0.31511976 |
| SMARCB1_Q368X | K562 | Mutant | 28z | −0.46137514 | −0.36617492 | −0.67273352 | NA | NA |
| SMARCB1_Q368X | K562 | Mutant | BBz | 0.38671299 | −0.04578918 | −0.71877212 | NA | −0.16349416 |
| SMARCB1_Q368X | K562-19 | Mutant | 28z | −0.71601326 | −0.79437677 | −0.68366037 | −0.34298667 | NA |
| SMARCB1_Q368X | K562-19 | Mutant | BBz | −0.27759608 | −0.70115249 | −0.65804064 | −0.26181593 | −0.322116 |
| SPI1-TCF7 | K562 | Fusion | 28z | −0.53040337 | −0.34621007 | −0.30420449 | NA | NA |
| SPI1-TCF7 | K562 | Fusion | BBz | 0.57235517 | −0.19235583 | −0.05235878 | NA | −0.16193225 |
| SPI1-TCF7 | K562-19 | Fusion | 28z | −0.26228599 | −0.4424889 | −0.54969392 | −0.25682659 | NA |
| SPI1-TCF7 | K562-19 | Fusion | BBz | −0.3100131 | −0.61710555 | −0.60310973 | −0.21940929 | −0.14846273 |
| STAT3 | K562 | Wild-type | 28z | −0.15080995 | −0.36655557 | −0.19152097 | NA | NA |
| STAT3 | K562 | Wild-type | BBz | 0.49677831 | −0.21131734 | 0.43387418 | NA | −0.16115093 |
| STAT3 | K562-19 | Wild-type | 28z | 0.08732942 | −0.04676817 | −0.16794432 | −0.32161814 | NA |
| STAT3 | K562-19 | Wild-type | BBz | −0.04533294 | −0.13170809 | 0.18183811 | −0.24073363 | −0.20237081 |
| STAT3_D661I | K562 | Mutant | 28z | 0.13870547 | −0.29602839 | 0.41702812 | NA | NA |
| STAT3_D661I | K562 | Mutant | BBz | −0.43614401 | −0.31233699 | 0.5539521 | NA | −0.13967422 |
| STAT3_D661I | K562-19 | Mutant | 28z | 0.45653491 | −0.00714714 | 0.42912679 | −0.29108867 | NA |
| STAT3_D661I | K562-19 | Mutant | BBZ | 0.20309873 | −0.12688344 | 0.39309856 | −0.26181593 | −0.10072875 |
| STAT3_G618R | K562 | Mutant | 28z | 0.39559527 | −0.38318303 | −0.00474699 | NA | NA |
| STAT3_G618R | K562 | Mutant | BBz | 0.03605599 | −0.37983119 | 0.42350786 | NA | −0.13771841 |
| STAT3_G618R | K562-19 | Mutant | 28z | −0.12323466 | −0.22181842 | 0.15247081 | −0.3398235 | NA |
| STAT3_G618R | K562-19 | Mutant | BBZ | 0.27884646 | −0.15143677 | 0.22034689 | −0.25164868 | −0.14557882 |
| STAT3_N647I | K562 | Mutant | 28z | −0.16335163 | −0.32806715 | −0.19958653 | NA | NA |
| STAT3_N647I | K562 | Mutant | BBz | −0.00774842 | −0.26404627 | 0.21457802 | NA | −0.12990961 |
| STAT3_N647I | K562-19 | Mutant | 28z | −0.00585759 | −0.19061317 | 0.26322985 | −0.3424531 | NA |
| STAT3_N647I | K562-19 | Mutant | BBz | 0.28169004 | −0.18301204 | 0.10589629 | −0.26181593 | −0.10731252 |
| STAT5B | K562 | Wild-type | 28z | −0.40487927 | −0.39818928 | −0.50856541 | NA | NA |
| STAT5B | K562 | Wild-type | BBz | 0.53914157 | −0.20078768 | −0.10050743 | NA | −0.1634936 |
| STAT5B | K562-19 | Wild-type | 28z | −0.17880463 | −0.32302214 | −0.32294574 | −0.31367221 | NA |
| STAT5B | K562-19 | Wild-type | BBz | −0.25081455 | −0.37433775 | −0.22820513 | −0.24893913 | −0.22582368 |
| STAT5B_T628S | K562 | Mutant | 28z | −0.59579927 | −0.37637061 | −0.45966002 | NA | NA |
| STAT5B_T628S | K562 | Mutant | BBz | 0.00178924 | −0.15911175 | −0.18991499 | NA | −0.15841722 |
| STAT5B_T628S | K562-19 | Mutant | 28z | −0.28776931 | −0.44463217 | −0.40412377 | −0.33107617 | NA |
| STAT5B_T628S | K562-19 | Mutant | BBz | −0.19976608 | −0.34071898 | −0.24247574 | −0.25164868 | −0.24434292 |
| STAT5B_Y665F | K562 | Mutant | 28z | −0.55909106 | −0.31539753 | −0.51453704 | NA | NA |
| STAT5B_Y665F | K562 | Mutant | BBz | 0.27637053 | −0.10626277 | −0.16922203 | NA | −0.16622861 |
| STAT5B_Y665F | K562-19 | Mutant | 28z | −0.31376501 | −0.32044038 | −0.69008188 | −0.30260997 | NA |
| STAT5B_Y665F | K562-19 | Mutant | BBz | −0.31075105 | −0.513038 | −0.54036276 | −0.25074722 | −0.23282391 |
| TNFRSF1B | K562 | Wild-type | 28z | −0.4745957 | 0.110219 | −0.2970961 | NA | NA |
| TNFRSF1B | K562 | Wild-type | BBz | −0.0403523 | 0.94032292 | 0.2084503 | NA | −0.15177781 |
| TNFRSF1B | K562-19 | Wild-type | 28z | −0.87041062 | 0.59058385 | −0.25496482 | −0.30627317 | NA |
| TNFRSF1B | K562-19 | Wild-type | BBz | −0.19335512 | 0.27300743 | −0.23121669 | −0.19391559 | −0.2443408 |
| TNFRSF1B_G256C | K562 | Mutant | 28z | −0.48399263 | 0.40692893 | −0.25995884 | NA | NA |
| TNFRSF1B_G256C | K562 | Mutant | BBz | 0.6930113 | 2.03443779 | 0.28157813 | NA | −0.13225321 |
| TNFRSF1B_G256C | K562-19 | Mutant | 28z | −0.35020921 | 0.92972811 | −0.08211524 | −0.23223905 | NA |
| TNFRSF1B_G256C | K562-19 | Mutant | BBz | −0.02784839 | 0.74686588 | −0.12709225 | −0.25434095 | −0.22088768 |
| TNFRSF1B_T377I | K562 | Mutant | 28z | −0.33576472 | 1.06097378 | −0.50435526 | NA | NA |
| TNFRSF1B_T377I | K562 | Mutant | BBz | 0.51757194 | 2.93772452 | −0.20383729 | NA | −0.14787192 |
| TNFRSF1B_T377I | K562-19 | Mutant | 28z | −0.16911799 | 1.21364477 | −0.52453902 | −0.03812957 | NA |
| TNFRSF1B_T377I | K562-19 | Mutant | BBz | −0.2620997 | 1.03465333 | −0.64377003 | −0.23336602 | −0.28508185 |
| TP53 | K562 | Wild-type | 28z | 0.14251044 | −0.17236928 | 0.01138413 | NA | NA |
| TP53 | K562 | Wild-type | BBz | −0.74044687 | −0.0501042 | −0.09734038 | NA | −0.14943476 |
| TP53 | K562-19 | Wild-type | 28z | −0.09228641 | 0.20709354 | −0.22786243 | −0.20325183 | NA |
| TP53 | K562-19 | Wild-type | BBz | −0.10002875 | −0.11519063 | −0.52200483 | −0.23889748 | −0.0739801 |
| TP53_R273P | K562 | Mutant | 28z | −0.69547482 | −0.39234249 | −0.46029367 | NA | NA |
| TP53_R273P | K562 | Mutant | BBz | −0.09641039 | −0.17315171 | −0.15547434 | NA | −0.15998051 |
| TP53_R273P | K562-19 | Mutant | 28z | −0.30416944 | −0.47095509 | −0.57591109 | −0.30374806 | NA |
| TP53_R273P | K562-19 | Mutant | BBz | −0.07456323 | −0.34071898 | −0.47101836 | −0.25344572 | −0.20360195 |
| VAV1 | K562 | Wild-type | 28z | 1.69015501 | −0.19419154 | 0.49339655 | NA | NA |
| VAV1 | K562 | Wild-type | BBz | 0.38771072 | −0.25044963 | 0.54144266 | NA | −0.09671506 |
| VAV1 | K562-19 | Wild-type | 28z | 0.19924322 | −0.22773503 | 0.25898077 | −0.3009065 | NA |
| VAV1 | K562-19 | Wild-type | BBz | 0.83381309 | 0.07656514 | 0.4638773 | −0.25344572 | −0.0081329 |
| VAV1_D797H | K562 | Mutant | 28z | 1.07978864 | −0.19517831 | 0.68582311 | NA | NA |
| VAV1_D797H | K562 | Mutant | BBz | −0.23880245 | −0.25206283 | 0.8836107 | NA | −0.11545778 |
| VAV1_D797H | K562-19 | Mutant | 28z | 0.38905639 | −0.30849115 | 0.42452363 | −0.33597073 | NA |
| VAV1_D797H | K562-19 | Mutant | BBz | 0.44839533 | 0.18150847 | 0.37631785 | −0.2480327 | −0.07932764 |
| VAV1_R798Q | K562 | Mutant | 28z | 0.83286034 | −0.20416535 | 0.61914673 | NA | NA |
| VAV1_R798Q | K562 | Mutant | BBz | 0.23384099 | −0.28740408 | 0.81943633 | NA | −0.10804196 |
| VAV1_R798Q | K562-19 | Mutant | 28z | 0.20522753 | −0.35800073 | 0.29963436 | −0.32318256 | NA |
| VAV1_R798Q | K562-19 | Mutant | BBZ | 0.66830636 | 0.33205532 | 0.46029144 | −0.2552337 | −0.12130254 |
| VAV1-GSS | K562 | Fusion | 28z | 0.99509265 | −0.21842428 | 0.32021909 | NA | NA |
| VAV1-GSS | K562 | Fusion | BBz | 0.9002231 | −0.23806478 | 0.49522281 | NA | −0.1463102 |
| VAV1-GSS | K562-19 | Fusion | 28z | 0.1197961 | −0.41402271 | −0.11209824 | −0.34298667 | NA |
| VAV1-GSS | K562-19 | Fusion | BBz | 0.8401066 | 0.33030375 | 0.42909725 | −0.2471248 | −0.11184598 |

TABLE 5
RESULTS OF IN VITRO MUTATION SCREENING (CONTINUED)

| construct | condition | group | CAR | ctrl_pval_nfat | ctrl_pval_nfkb | ctrl_pval_ap1 | ctrl_pval_il2 |
|---|---|---|---|---|---|---|---|
| BCL6 | K562 | Wild-type | 28z | 1 | 1 | 1 | NA |
| BCL6 | K562 | Wild-type | BBz | 1 | 1 | 1 | NA |
| BCL6 | K562-19 | Wild-type | 28z | 1 | 0.001197 | 0.423738 | 1 |
| BCL6 | K562-19 | Wild-type | BBz | 1 | 0.761292 | 1 | 1 |
| BCL6_ | K562 | Mutant | 28z | 1 | 1 | 1 | NA |
| S647R | | | | | | | |
| BCL6_ | K562 | Mutant | BBz | 1 | 0.912114 | 1 | NA |
| S647R | | | | | | | |
| BCL6_ | K562-19 | Mutant | 28z | 6.39E−19 | 3.90E−09 | 1 | 1 |
| S647R | | | | | | | |
| BCL6_ | K562-19 | Mutant | BBz | 0.4788 | 1 | 1 | 1 |
| S647R | | | | | | | |
| BRAF | K562 | Wild-type | 28z | 0.241794 | 1 | 1 | NA |
| BRAF | K562 | Wild-type | BBz | 1 | 1 | 1 | NA |
| BRAF | K562-19 | Wild-type | 28z | 2.01E−36 | 4.02E−20 | 2.32E−29 | 1 |
| BRAF | K562-19 | Wild-type | BBz | 1.51E−06 | 2.17E−13 | 4.12E−08 | 1 |
| BRAF_ | K562 | Mutant | 28z | 1 | 1 | 1 | NA |
| D594N | | | | | | | |
| BRAF_ | K562 | Mutant | BBz | 1 | 0.256158 | 1 | NA |
| D594N | | | | | | | |
| BRAF_ | K562-19 | Mutant | 28z | 6.37E−32 | 3.50E−56 | 2.61E−20 | 2.20E−05 |
| D594N | | | | | | | |
| BRAF_ | K562-19 | Mutant | BBz | 5.24E−13 | 3.71E−24 | 4.57E−18 | 1 |
| D594N | | | | | | | |
| BRAF_ | K562 | Mutant | 28z | 3.45E−61 | 1.11E−71 | 3.09E−64 | NA |
| G469A | | | | | | | |
| BRAF_ | K562 | Mutant | BBz | 0.043092 | 1.02E−46 | 1.57E−46 | NA |
| G469A | | | | | | | |
| BRAF_ | K562-19 | Mutant | 28z | 5.70E−209 | 1.36E−127 | 4.74E−99 | 3.18E−129 |
| G469A | | | | | | | |
| BRAF_ | K562-19 | Mutant | BBz | 2.28E−137 | 2.13E−103 | 6.75E−97 | 1.25E−10 |
| G469A | | | | | | | |
| BRAF_ | K562 | Mutant | 28z | 3.57E−13 | 1 | 1 | NA |
| G469R | | | | | | | |
| BRAF_ | K562 | Mutant | BBz | 1 | 0.0684684 | 1 | NA |
| G469R | | | | | | | |
| BRAF_ | K562-19 | Mutant | 28z | 3.85E−56 | 1.66E−79 | 2.97E−60 | 0.00010855 |
| G469R | | | | | | | |
| BRAF_ | K562-19 | Mutant | BBz | 1.27E−28 | 1.02E−37 | 7.59E−57 | 1 |
| G469R | | | | | | | |
| CARD11 | K562 | Wild-type | 28z | 0.00030883 | 9.36E−36 | 1 | NA |
| CARD11 | K562 | Wild-type | BBz | 1 | 1 | 1 | NA |
| CARD11 | K562-19 | Wild-type | 28z | 4.40E−08 | 8.38E−120 | 2.75E−47 | 1.85E−07 |
| CARD11 | K562-19 | Wild-type | BBz | 1 | 1.06E−49 | 4.57E−06 | 1 |
| CARD11_ | K562 | Mutant | 28z | 6.85E−05 | 8.36E−167 | 0.1726074 | NA |
| D357N | | | | | | | |
| CARD11_ | K562 | Mutant | BBz | 1 | 2.39E−63 | 1 | NA |
| D357N | | | | | | | |
| CARD11_ | K562-19 | Mutant | 28z | 4.19E−11 | 6.22E−168 | 6.94E−117 | 6.88E−101 |
| D357N | | | | | | | |
| CARD11_ | K562-19 | Mutant | BBZ | 1 | 7.18E−122 | 8.93E−68 | 0.0100868 |
| D357N | | | | | | | |
| CARD11_ | K562 | Mutant | 28z | 0.0128079 | 3.57E−33 | 1 | NA |
| E634K: | | | | | | | |
| S655C | | | | | | | |
| CARD11_ | K562 | Mutant | BBz | 1 | 1 | 1 | NA |
| E634K: | | | | | | | |
| S655C | | | | | | | |
| CARD11_ | K562-19 | Mutant | 28z | 0.081396 | 1.40E−134 | 3.21E−68 | 8.32E−12 |
| E634K: | | | | | | | |
| S655C | | | | | | | |
| CARD11_ | K562-19 | Mutant | BBZ | 1 | 3.54E−87 | 9.29E−25 | 1 |
| E634K: | | | | | | | |
| S655C | | | | | | | |
| CARD11_ | K562 | Mutant | 28z | 1 | 2.66E−55 | 1 | NA |
| S615F | | | | | | | |
| CARD11_ | K562 | Mutant | BBz | 1 | 1 | 1 | NA |
| S615F | | | | | | | |
| CARD11_ | K562-19 | Mutant | 28z | 0.00175241 | 2.68E−169 | 4.00E−121 | 6.91E−40 |
| S615F | | | | | | | |
| CARD11_ | K562-19 | Mutant | BBz | 1 | 1.33E−117 | 5.03E−82 | 1 |
| S615F | | | | | | | |
| CARD11_ | K562 | Mutant | 28z | 0.0113715 | 3.42E−154 | 0.00046683 | NA |
| Y361C | | | | | | | |
| CARD11_ | K562 | Mutant | BBz | 1 | 6.61E−32 | 1 | NA |
| Y361C | | | | | | | |
| CARD11_ | K562-19 | Mutant | 28z | 2.13E−05 | 5.72E−173 | 1.14E−139 | 4.31E−106 |
| Y361C | | | | | | | |
| CARD11_ | K562-19 | Mutant | BBz | 1 | 1.24E−140 | 2.54E−134 | 1.13E−05 |
| Y361C | | | | | | | |
| CARD11- | K562 | Fusion | 28z | 0.0013957 | 5.17E−231 | 0.519498 | NA |
| PIK3R3 | | | | | | | |
| CARD11- | K562 | Fusion | BBz | 1 | 1.64E−138 | 1 | NA |
| PIK3R3 | | | | | | | |
| CARD11- | K562-19 | Fusion | 28z | 8.45E−46 | 3.09E−171 | 0.00044528 | 4.18E−113 |
| PIK3R3 | | | | | | | |
| CARD11- | K562-19 | Fusion | BBz | 0.00016806 | 3.61E−147 | 6.27E−95 | 1.05E−08 |
| PIK3R3 | | | | | | | |
| CARMIL2_ | K562 | Mutant | 28z | 1.67E−16 | 1 | 1 | NA |
| Q575E | | | | | | | |
| CARMIL2_ | K562 | Mutant | BBz | 1 | 1 | 1 | NA |
| Q575E | | | | | | | |
| CARMIL2_ | K562-19 | Mutant | 28z | 3.76E−13 | 0.061047 | 2.29E−08 | 1 |
| Q575E | | | | | | | |
| CARMIL2_ | K562-19 | Mutant | BBz | 1 | 1 | 0.81396 | 1 |
| Q575E | | | | | | | |
| CCND3 | K562 | Wild-type | 28z | 1 | 1 | 1 | NA |
| CCND3 | K562 | Wild-type | BBz | 1 | 1 | 1 | NA |
| CCND3 | K562-19 | Wild-type | 28z | 1.20E−23 | 2.32E−28 | 1.21E−17 | 1 |
| CCND3 | K562-19 | Wild-type | BBz | 0.044289 | 0.1393308 | 1.02E−19 | 1 |
| CCND3_ | K562 | Mutant | 28z | 1 | 1 | 1 | NA |
| P284S | | | | | | | |
| CCND3_ | K562 | Mutant | BBz | 0.246582 | 0.0761292 | 1 | NA |
| P284S | | | | | | | |
| CCND3_ | K562-19 | Mutant | 28z | 1.11E−33 | 4.86E−39 | 4.52E−22 | 1 |
| P284S | | | | | | | |
| CCND3_ | K562-19 | Mutant | BBz | 9.50E−11 | 2.18E−09 | 6.03E−22 | 1 |
| P284S | | | | | | | |
| CD28 | K562 | Wild-type | 28z | 1 | 1 | 1 | NA |
| CD28 | K562 | Wild-type | BBz | 1 | 1 | 1 | NA |
| CD28 | K562-19 | Wild-type | 28z | 1 | 1 | 1 | 1 |
| CD28 | K562-19 | Wild-type | BBz | 1 | 1 | 1 | 1 |
| CD28_ | K562 | Mutant | 28z | 1 | 1 | 1 | NA |
| F51I | | | | | | | |
| CD28_ | K562 | Mutant | BBz | 1 | 1 | 1 | NA |
| F51I | | | | | | | |
| CD28_ | K562-19 | Mutant | 28z | 1 | 0.0306432 | 1 | 1 |
| F51I | | | | | | | |
| CD28_ | K562-19 | Mutant | BBz | 1 | 1 | 1 | 1 |
| F51I | | | | | | | |
| CD28_ | K562 | Mutant | 28z | 1 | 1 | 1 | NA |
| F51V | | | | | | | |
| CD28_ | K562 | Mutant | BBz | 1 | 1 | 1 | NA |
| F51V | | | | | | | |
| CD28_ | K562-19 | Mutant | 28z | 1 | 0.00020469 | 1 | 1 |
| F51V | | | | | | | |
| CD28_ | K562-19 | Mutant | BBz | 1 | 1 | 1 | 1 |
| F51V | | | | | | | |
| CD28_ | K562 | Mutant | 28z | 1 | 1 | 1 | NA |
| Q77P | | | | | | | |
| CD28_ | K562 | Mutant | BBz | 1 | 1 | 1 | NA |
| Q77P | | | | | | | |
| CD28_ | K562-19 | Mutant | 28z | 1 | 1 | 1 | 1 |
| Q77P | | | | | | | |
| CD28_ | K562-19 | Mutant | BBz | 1 | 1 | 1 | 1 |
| Q77P | | | | | | | |
| CD28_ | K562 | Mutant | 28z | 1 | 1 | 1 | NA |
| T195P | | | | | | | |
| CD28_ | K562 | Mutant | BBz | 1 | 1 | 1 | NA |
| T195P | | | | | | | |
| CD28_ | K562-19 | Mutant | 28z | 1.92E−08 | 1 | 0.84987 | 1 |
| T195P | | | | | | | |
| CD28_ | K562-19 | Mutant | BBz | 1 | 1 | 0.301644 | 1 |
| T195P | | | | | | | |
| CD28- | K562 | Fusion | 28z | 1 | 1 | 1 | NA |
| CTLA4 | | | | | | | |
| CD28- | K562 | Fusion | BBz | 1 | 1 | 1 | NA |
| CTLA4 | | | | | | | |
| CD28- | K562-19 | Fusion | 28z | 1 | 1 | 1 | 1 |
| CTLA4 | | | | | | | |
| CD28- | K562-19 | Fusion | BBz | 1 | 1 | 1 | 1 |
| CTLA4 | | | | | | | |
| CD3E | K562 | Wild-type | 28z | 1 | 1 | 1 | NA |
| CD3E | K562 | Wild-type | BBz | 1 | 0.847476 | 1 | NA |
| CD3E | K562-19 | Wild-type | 28z | 1 | 1 | 1 | 1 |
| CD3E | K562-19 | Wild-type | BBz | 1 | 1 | 1 | 1 |
| CD3E_ | K562 | Mutant | 28z | 1 | 1 | 1 | NA |
| S41C | | | | | | | |
| CD3E_ | K562 | Mutant | BBz | 1 | 1 | 1 | NA |
| S41C | | | | | | | |
| CD3E_ | K562-19 | Mutant | 28z | 1 | 1 | 1 | 1 |
| S41C | | | | | | | |
| CD3E_ | K562-19 | Mutant | BBz | 1 | 1 | 1 | 1 |
| S41C | | | | | | | |
| CD53 | K562 | Wild-type | 28z | 1 | 1 | 1 | NA |
| CD53 | K562 | Wild-type | BBz | 1 | 1 | 1 | NA |
| CD53 | K562-19 | Wild-type | 28z | 1 | 1 | 1 | 1 |
| CD53 | K562-19 | Wild-type | BBz | 1 | 1 | 1 | 1 |
| CSNK1A1 | K562 | Wild-type | 28z | 1 | 1 | 1 | NA |
| CSNK1A1 | K562 | Wild-type | BBz | 1 | 1 | 1 | NA |
| CSNK1A1 | K562-19 | Wild-type | 28z | 1 | 1 | 1 | 1 |
| CSNK1A1 | K562-19 | Wild-type | BBz | 1 | 1 | 1 | 1 |
| CSNK1A1_ | K562 | Mutant | 28z | 1 | 1 | 1 | NA |
| S27C | | | | | | | |
| CSNK1A1_ | K562 | Mutant | BBz | 1 | 1 | 1 | NA |
| S27C | | | | | | | |
| CSNK1A1_ | K562-19 | Mutant | 28z | 1 | 1 | 1 | 1 |
| S27C | | | | | | | |
| CSNK1A1_ | K562-19 | Mutant | BBz | 1 | 1 | 1 | 1 |
| S27C | | | | | | | |
| CSNK2B | K562 | Wild-type | 28z | 1 | 1 | 1 | NA |
| CSNK2B | K562 | Wild-type | BBz | 1 | 1 | 1 | NA |
| CSNK2B | K562-19 | Wild-type | 28z | 1 | 1 | 1 | 1 |
| CSNK2B | K562-19 | Wild-type | BBz | 1 | 1 | 1 | 1 |
| CSNK2B_ | K562 | Mutant | 28z | 1 | 1 | 1 | NA |
| Q182X | | | | | | | |
| CSNK2B_ | K562 | Mutant | BBz | 1 | 1 | 1 | NA |
| Q182X | | | | | | | |
| CSNK2B_ | K562-19 | Mutant | 28z | 1 | 0.0246582 | 1 | 1 |
| Q182X | | | | | | | |
| CSNK2B_ | K562-19 | Mutant | BBz | 1 | 1 | 1 | 1 |
| Q182X | | | | | | | |
| DGKA | K562 | Wild-type | 28z | 1 | 1 | 1 | NA |
| DGKA | K562 | Wild-type | BBz | 1 | 1 | 1 | NA |
| DGKA | K562-19 | Wild-type | 28z | 1 | 1 | 1 | 1 |
| DGKA | K562-19 | Wild-type | BBz | 1 | 1 | 1 | 1 |
| DGKZ | K562 | Wild-type | 28z | 1 | 1 | 1 | NA |
| DGKZ | K562 | Wild-type | BBz | 1 | 1 | 1 | NA |
| DGKZ | K562-19 | Wild-type | 28z | 1.33E−37 | 4.69E−71 | 8.69E−19 | 1 |
| DGKZ | K562-19 | Wild-type | BBz | 0.00447678 | 2.59E−28 | 3.14E−15 | 1 |
| ECSIT | K562 | Wild-type | 28z | 1 | 1 | 1 | NA |
| ECSIT | K562 | Wild-type | BBz | 1 | 1 | 1 | NA |
| ECSIT | K562-19 | Wild-type | 28z | 1 | 1 | 1 | 1 |
| ECSIT | K562-19 | Wild-type | BBz | 1 | 1 | 1 | 1 |
| ECSIT_ | K562 | Mutant | 28z | 1 | 1 | 1 | NA |
| V140A | | | | | | | |
| ECSIT_ | K562 | Mutant | BBz | 1 | 1 | 1 | NA |
| V140A | | | | | | | |
| ECSIT_ | K562-19 | Mutant | 28z | 1 | 1 | 1 | 1 |
| V140A | | | | | | | |
| ECSIT_ | K562-19 | Mutant | BBz | 1 | 1 | 1 | 1 |
| V140A | | | | | | | |
| EIFS1 | K562 | Wild-type | 28z | 1 | 1 | 1 | NA |
| EIFS1 | K562 | Wild-type | BBz | 1 | 1 | 1 | NA |
| EIFS1 | K562-19 | Wild-type | 28z | 1 | 1 | 1 | 1 |
| EIFS1 | K562-19 | Wild-type | BBz | 1 | 0.00397404 | 0.2001384 | 1 |
| EIFS1_ | K562 | Mutant | 28z | 1 | 1 | 1 | NA |
| R89I | | | | | | | |
| EIFS1_ | K562 | Mutant | BBz | 1 | 1 | 1 | NA |
| R89I | | | | | | | |
| EIFS1_ | K562-19 | Mutant | 28z | 1 | 1 | 1 | 1 |
| R89I | | | | | | | |
| EIFS1_ | K562-19 | Mutant | BBz | 1 | 1 | 1 | 1 |
| R89I | | | | | | | |
| FYN | K562 | Wild-type | 28z | 1 | 1 | 1 | NA |
| FYN | K562 | Wild-type | BBz | 1 | 1 | 1 | NA |
| FYN | K562-19 | Wild-type | 28z | 0.00318402 | 2.22E−23 | 0.01022238 | 1 |
| FYN | K562-19 | Wild-type | BBz | 1 | 0.01970262 | 0.452466 | 1 |
| FYN_ | K562 | Mutant | 28z | 0.00040459 | 0.0591318 | 1 | NA |
| R86G | | | | | | | |
| FYN_ | K562 | Mutant | BBz | 1 | 1 | 1 | NA |
| R86G | | | | | | | |
| FYN_ | K562-19 | Mutant | 28z | 1 | 0.1630314 | 0.763686 | 1 |
| R86G | | | | | | | |
| FYN_ | K562-19 | Mutant | BBz | 0.0567378 | 5.41E−09 | 2.97E−08 | 1 |
| R86G | | | | | | | |
| FYN_ | K562 | Mutant | 28z | 1 | 1 | 1 | NA |
| Y531H | | | | | | | |
| FYN_ | K562 | Mutant | BBz | 1 | 1 | 1 | NA |
| Y531H | | | | | | | |
| FYN_ | K562-19 | Mutant | 28z | 1 | 1.97E−12 | 0.0988722 | 1 |
| Y531H | | | | | | | |
| FYN_ | K562-19 | Mutant | BBz | 1 | 3.69E−08 | 0.1623132 | 1 |
| Y531H | | | | | | | |
| GATA3 | K562 | Wild-type | 28z | 1 | 1 | 1 | NA |
| GATA3 | K562 | Wild-type | BBz | 1 | 1 | 1 | NA |
| GATA3 | K562-19 | Wild-type | 28z | 2.56E−10 | 1.05E−10 | 1 | 1 |
| GATA3 | K562-19 | Wild-type | BBz | 1 | 0.0469224 | 1 | 1 |
| GATA3_ | K562 | Mutant | 28z | 1 | 1 | 1 | NA |
| Y63X | | | | | | | |
| GATA3_ | K562 | Mutant | BBz | 1 | 1 | 1 | NA |
| Y63X | | | | | | | |
| GATA3_ | K562-19 | Mutant | 28z | 1 | 1 | 1 | 1 |
| Y63X | | | | | | | |
| GATA3_ | K562-19 | Mutant | BBz | 1 | 1 | 0.043092 | 1 |
| Y63X | | | | | | | |
| GNAQ | K562 | Wild-type | 28z | 1 | 1 | 1 | NA |
| GNAQ | K562 | Wild-type | BBz | 1 | 1 | 1 | NA |
| GNAQ | K562-19 | Wild-type | 28z | 1 | 1 | 1 | 1 |
| GNAQ | K562-19 | Wild-type | BBz | 1 | 1 | 1 | 1 |
| GNAQ_ | K562 | Mutant | 28z | 1 | 1 | 1 | NA |
| T96S | | | | | | | |
| GNAQ_ | K562 | Mutant | BBz | 1 | 1 | 1 | NA |
| T96S | | | | | | | |
| GNAQ_ | K562-19 | Mutant | 28z | 1 | 1 | 1 | 1 |
| T96S | | | | | | | |
| GNAQ_ | K562-19 | Mutant | BBZ | 1 | 1 | 1 | 1 |
| T96S | | | | | | | |
| ICOS- | K562 | Fusion | 28z | 1 | 1 | 1 | NA |
| CD28 | | | | | | | |
| ICOS- | K562 | Fusion | BBz | 1 | 1 | 1 | NA |
| CD28 | | | | | | | |
| ICOS- | K562-19 | Fusion | 28z | 1 | 1 | 1 | 1 |
| CD28 | | | | | | | |
| ICOS- | K562-19 | Fusion | BBz | 1 | 1 | 1 | 1 |
| CD28 | | | | | | | |
| IRF4 | K562 | Wild-type | 28z | 1 | 1 | 1 | NA |
| IRF4 | K562 | Wild-type | BBz | 1 | 1 | 1 | NA |
| IRF4 | K562-19 | Wild-type | 28z | 1.10E−16 | 1 | 1 | 1 |
| IRF4 | K562-19 | Wild-type | BBz | 8.47E−07 | 1 | 1 | 1 |
| IRF4_ | K562 | Mutant | 28z | 1 | 1 | 1 | NA |
| K59R | | | | | | | |
| IRF4_ | K562 | Mutant | BBz | 1 | 1 | 1 | NA |
| K59R | | | | | | | |
| IRF4_ | K562-19 | Mutant | 28z | 4.09E−15 | 1.07E−32 | 1.90E−30 | 1 |
| K59R | | | | | | | |
| IRF4_ | K562-19 | Mutant | BBz | 1.27E−07 | 1 | 1 | 1 |
| K59R | | | | | | | |
| ITGB2 | K562 | Wild-type | 28z | 1 | 1 | 1 | NA |
| ITGB2 | K562 | Wild-type | BBz | 1 | 0.641592 | 1 | NA |
| ITGB2 | K562-19 | Wild-type | 28z | 1 | 1 | 1 | 1 |
| ITGB2 | K562-19 | Wild-type | BBz | 1 | 1 | 1 | 1 |
| ITGB2_ | K562 | Mutant | 28z | 1 | 1 | 1 | NA |
| E234K | | | | | | | |
| ITGB2_ | K562 | Mutant | BBz | 1 | 1 | 1 | NA |
| E234K | | | | | | | |
| ITGB2_ | K562-19 | Mutant | 28z | 1 | 1 | 1 | 1 |
| E234K | | | | | | | |
| ITGB2_ | K562-19 | Mutant | BBz | 1 | 1 | 1 | 1 |
| E234K | | | | | | | |
| ITK- | K562 | Fusion | 28z | 1 | 1 | 1 | NA |
| FER | | | | | | | |
| ITK- | K562 | Fusion | BBz | 1 | 1 | 1 | NA |
| FER | | | | | | | |
| ITK- | K562-19 | Fusion | 28z | 0.00066553 | 1 | 1 | 1 |
| FER | | | | | | | |
| ITK- | K562-19 | Fusion | BBz | 0.00089057 | 0.1989414 | 0.0687078 | 1 |
| FER | | | | | | | |
| ITK- | K562 | Fusion | 28z | 1 | 1 | 1 | NA |
| SYK | | | | | | | |
| ITK- | K562 | Fusion | BBz | 1 | 1 | 1 | NA |
| SYK | | | | | | | |
| ITK- | K562-19 | Fusion | 28z | 1 | 1 | 1 | 1 |
| SYK | | | | | | | |
| ITK- | K562-19 | Fusion | BBz | 1 | 1 | 1 | 1 |
| SYK | | | | | | | |
| JAK1_ | K562 | Mutant | 28z | 0.1671012 | 1 | 1 | NA |
| G1097A | | | | | | | |
| JAK1_ | K562 | Mutant | BBz | 1 | 1 | 1 | NA |
| G1097A | | | | | | | |
| JAK1_ | K562-19 | Mutant | 28z | 1.03E−05 | 0.0924084 | 4.43E−08 | 1 |
| G1097A | | | | | | | |
| JAK1_ | K562-19 | Mutant | BBz | 1 | 0.241794 | 0.0440496 | 1 |
| G1097A | | | | | | | |
| JAK3 | K562 | Wild-type | 28z | 1 | 1 | 1 | NA |
| JAK3 | K562 | Wild-type | BBz | 1 | 1 | 1 | NA |
| JAK3 | K562-19 | Wild-type | 28z | 1 | 1 | 1 | 1 |
| JAK3 | K562-19 | Wild-type | BBZ | 1 | 1 | 1 | 1 |
| JAK3_ | K562 | Mutant | 28z | 1 | 1 | 1 | NA |
| A573V | | | | | | | |
| JAK3_ | K562 | Mutant | BBz | 1 | 1 | 1 | NA |
| A573V | | | | | | | |
| JAK3_ | K562-19 | Mutant | 28z | 1 | 0.253764 | 0.576954 | 1 |
| A573V | | | | | | | |
| JAK3_ | K562-19 | Mutant | BBz | 1 | 1 | 1 | 1 |
| A573V | | | | | | | |
| JUNB | K562 | Wild-type | 28z | 1 | 1 | 1 | NA |
| JUNB | K562 | Wild-type | BBz | 1 | 1 | 1 | NA |
| JUNB | K562-19 | Wild-type | 28z | 2.04E−23 | 1 | 0.34713 | 1 |
| JUNB | K562-19 | Wild-type | BBz | 1 | 1 | 3.54E−13 | 1 |
| JUNB_ | K562 | Mutant | 28z | 1 | 1 | 1 | NA |
| A282V | | | | | | | |
| JUNB_ | K562 | Mutant | BBz | 1 | 1 | 1 | NA |
| A282V | | | | | | | |
| JUNB_ | K562-19 | Mutant | 28z | 1 | 1 | 2.51E−16 | 1 |
| A282V | | | | | | | |
| JUNB_ | K562-19 | Mutant | BBz | 1 | 1 | 4.19E−20 | 1 |
| A282V | | | | | | | |
| KCNQ1 | K562 | Wild-type | 28z | 1 | 1 | 1 | NA |
| KCNQ1 | K562 | Wild-type | BBz | 1 | 1 | 1 | NA |
| KCNQ1 | K562-19 | Wild-type | 28z | 1 | 1 | 1 | 1 |
| KCNQ1 | K562-19 | Wild-type | BBz | 1 | 1 | 1 | 1 |
| KCNQ1_ | K562 | Mutant | 28z | 1 | 1 | 1 | NA |
| R583C | | | | | | | |
| KCNQ1_ | K562 | Mutant | BBz | 1 | 1 | 1 | NA |
| R583C | | | | | | | |
| KCNQ1_ | K562-19 | Mutant | 28z | 1 | 1 | 1 | 1 |
| R583C | | | | | | | |
| KCNQ1_ | K562-19 | Mutant | BBz | 1 | 1 | 1 | 1 |
| R583C | | | | | | | |
| MSC | K562 | Wild-type | 28z | 1 | 1 | 1 | NA |
| MSC | K562 | Wild-type | BBz | 1 | 1 | 1 | NA |
| MSC | K562-19 | Wild-type | 28z | 1.30E−09 | 3.06E−34 | 3.04E−05 | 1 |
| MSC | K562-19 | Wild-type | BBz | 1 | 2.14E−12 | 0.00031361 | 1 |
| MSC_ | K562 | Mutant | 28z | 1 | 1 | 1 | NA |
| E116K | | | | | | | |
| MSC_ | K562 | Mutant | BBZ | 1 | 1 | 1 | NA |
| E116K | | | | | | | |
| MSC_ | K562-19 | Mutant | 28z | 3.93E−13 | 1 | 1 | 1 |
| E116K | | | | | | | |
| MSC_ | K562-19 | Mutant | BBz | 2.63E−05 | 1 | 7.97E−05 | 1 |
| E116K | | | | | | | |
| MYCN | K562 | Wild-type | 28z | 1 | 1 | 1 | NA |
| MYCN | K562 | Wild-type | BBz | 1 | 1 | 1 | NA |
| MYCN | K562-19 | Wild-type | 28z | 3.38E−14 | 1.55E−14 | 0.00019631 | 1 |
| MYCN | K562-19 | Wild-type | BBz | 1 | 1 | 4.55E−05 | 1 |
| MYCN_ | K562 | Mutant | 28z | 1 | 1 | 1 | NA |
| P44L | | | | | | | |
| MYCN_ | K562 | Mutant | BBz | 1 | 1 | 1 | NA |
| P44L | | | | | | | |
| MYCN_ | K562-19 | Mutant | 28z | 9.74E−16 | 1.55E−25 | 1.97E−08 | 1 |
| P44L | | | | | | | |
| MYCN_ | K562-19 | Mutant | BBz | 1 | 1 | 7.09E−07 | 1 |
| P44L | | | | | | | |
| NFKB1 | K562 | Wild-type | 28z | 1 | 1 | 1 | NA |
| NFKB1 | K562 | Wild-type | BBz | 1 | 1 | 1 | NA |
| NFKB1 | K562-19 | Wild-type | 28z | 1.04E−10 | 1.00E−23 | 0.01474704 | 1 |
| NFKB1 | K562-19 | Wild-type | BBz | 6.85E−05 | 6.85E−11 | 4.69E−05 | 1 |
| NFKB1_ | K562 | Mutant | 28z | 1.63E−06 | 1 | 1 | NA |
| H67Y | | | | | | | |
| NFKB1_ | K562 | Mutant | BBz | 1 | 1 | 1 | NA |
| H67Y | | | | | | | |
| NFKB1_ | K562-19 | Mutant | 28z | 0.0265734 | 5.31E−15 | 4.62E−12 | 1 |
| H67Y | | | | | | | |
| NFKB1_ | K562-19 | Mutant | BBz | 1.54E−07 | 0.1989414 | 8.98E−17 | 1 |
| H67Y | | | | | | | |
| NFKB2 | K562 | Wild-type | 28z | 1 | 1 | 1 | NA |
| NFKB2 | K562 | Wild-type | BBz | 1 | 0.256158 | 1 | NA |
| NFKB2 | K562-19 | Wild-type | 28z | 1 | 4.88E−22 | 1 | 1 |
| NFKB2 | K562-19 | Wild-type | BBz | 0.0928872 | 6.22E−07 | 0.00871416 | 1 |
| NFKB2_ | K562 | Mutant | 28z | 1 | 1 | 1 | NA |
| K656X | | | | | | | |
| NFKB2_ | K562 | Mutant | BBz | 1 | 1 | 1 | NA |
| K656X | | | | | | | |
| NFKB2_ | K562-19 | Mutant | 28z | 1 | 1 | 1 | 1 |
| K656X | | | | | | | |
| NFKB2_ | K562-19 | Mutant | BBz | 1 | 1 | 1 | 1 |
| K656X | | | | | | | |
| NPM- | K562 | Fusion | 28z | 1 | 1 | 1 | NA |
| ALK | | | | | | | |
| NPM- | K562 | Fusion | BBz | 1 | 1 | 1 | NA |
| ALK | | | | | | | |
| NPM- | K562-19 | Fusion | 28z | 1 | 1 | 1.66E−24 | 1 |
| ALK | | | | | | | |
| NPM- | K562-19 | Fusion | BBz | 1 | 1 | 2.73E−11 | 1 |
| ALK | | | | | | | |
| NPM- | K562 | Fusion | 28z | 1 | 1 | 1 | NA |
| TYK | | | | | | | |
| NPM- | K562 | Fusion | BBz | 1 | 1 | 1 | NA |
| TYK | | | | | | | |
| NPM- | K562-19 | Fusion | 28z | 2.56E−10 | 1 | 1.27E−10 | 1 |
| TYK | | | | | | | |
| NPM- | K562-19 | Fusion | BBz | 2.56E−08 | 0.0306432 | 3.69E−20 | 1 |
| TYK | | | | | | | |
| NRAS | K562 | Wild-type | 28z | 1 | 1 | 1 | NA |
| NRAS | K562 | Wild-type | BBz | 1 | 1 | 1 | NA |
| NRAS | K562-19 | Wild-type | 28z | 1 | 1 | 1 | 1 |
| NRAS | K562-19 | Wild-type | BBz | 1 | 1 | 1 | 1 |
| NRAS_ | K562 | Mutant | 28z | 1 | 1 | 1 | NA |
| G13D | | | | | | | |
| NRAS_ | K562 | Mutant | BBz | 1 | 1 | 1 | NA |
| G13D | | | | | | | |
| NRAS_ | K562-19 | Mutant | 28z | 0.00094802 | 0.241794 | 1 | 1 |
| G13D | | | | | | | |
| NRAS_ | K562-19 | Mutant | BBz | 1 | 1 | 1 | 1 |
| G13D | | | | | | | |
| NRAS_ | K562 | Mutant | 28z | 1 | 1 | 1 | NA |
| Q61H | | | | | | | |
| NRAS_ | K562 | Mutant | BBZ | 1 | 1 | 1 | NA |
| Q61H | | | | | | | |
| NRAS_ | K562-19 | Mutant | 28z | 4.19E−08 | 1.05E−05 | 1 | 1 |
| Q61H | | | | | | | |
| NRAS_ | K562-19 | Mutant | BBz | 1 | 1 | 1 | 1 |
| Q61H | | | | | | | |
| PDCD1 | K562 | Wild-type | 28z | 1 | 1 | 1 | NA |
| PDCD1 | K562 | Wild-type | BBz | 1 | 1 | 1 | NA |
| PDCD1 | K562-19 | Wild-type | 28z | 1 | 1 | 1 | 1 |
| PDCD1 | K562-19 | Wild-type | BBz | 1 | 1 | 0.1587222 | 1 |
| PDCD1_ | K562 | Mutant | 28z | 1 | 1 | 1 | NA |
| R231X | | | | | | | |
| PDCD1_ | K562 | Mutant | BBz | 1 | 1 | 1 | NA |
| R231X | | | | | | | |
| PDCD1_ | K562-19 | Mutant | 28z | 1 | 1 | 1 | 1 |
| R231X | | | | | | | |
| PDCD1_ | K562-19 | Mutant | BBz | 1 | 1 | 1 | 1 |
| R231X | | | | | | | |
| PLCG1 | K562 | Wild-type | 28z | 1 | 1 | 1 | NA |
| PLCG1 | K562 | Wild-type | BBz | 1 | 1 | 1 | NA |
| PLCG1 | K562-19 | Wild-type | 28z | 1 | 1 | 1 | 1 |
| PLCG1 | K562-19 | Wild-type | BBz | 0.1671012 | 1 | 1 | 1 |
| PLCG1_ | K562 | Mutant | 28z | 7.01E−80 | 1.41E−23 | 0.00179311 | NA |
| D1165H | | | | | | | |
| PLCG1_ | K562 | Mutant | BBz | 1 | 1 | 1 | NA |
| D1165H | | | | | | | |
| PLCG1_ | K562-19 | Mutant | 28z | 1.06E−66 | 1.11E−90 | 3.02E−39 | 0.111222 |
| D1165H | | | | | | | |
| PLCG1_ | K562-19 | Mutant | BBz | 1.50E−88 | 1.81E−49 | 2.80E−78 | 1 |
| D1165H | | | | | | | |
| PLCG1_ | K562 | Mutant | 28z | 1.51E−06 | 0.00113236 | 1 | NA |
| E1163K | | | | | | | |
| PLCG1_ | K562 | Mutant | BBz | 1 | 1 | 1 | NA |
| E1163K | | | | | | | |
| PLCG1_ | K562-19 | Mutant | 28z | 2.25E−57 | 1.06E−85 | 2.66E−24 | 7.95E−16 |
| E1163K | | | | | | | |
| PLCG1_ | K562-19 | Mutant | BBz | 8.07E−52 | 9.46E−36 | 1.33E−38 | 1 |
| E1163K | | | | | | | |
| PLCG1_ | K562 | Mutant | 28z | 0.1584828 | 1 | 1 | NA |
| E47K | | | | | | | |
| PLCG1_ | K562 | Mutant | BBz | 1 | 1 | 1 | NA |
| E47K | | | | | | | |
| PLCG1_ | K562-19 | Mutant | 28z | 5.39E−37 | 8.91E−59 | 5.77E−07 | 1.34E−08 |
| E47K | | | | | | | |
| PLCG1_ | K562-19 | Mutant | BBz | 3.71E−19 | 4.24E−17 | 2.87E−07 | 1 |
| E47K | | | | | | | |
| PLCG1_ | K562 | Mutant | 28z | 1.69E−09 | 7.21E−09 | 1 | NA |
| R48W | | | | | | | |
| PLCG1_ | K562 | Mutant | BBz | 1 | 1 | 1 | NA |
| R48W | | | | | | | |
| PLCG1_ | K562-19 | Mutant | 28z | 1.82E−85 | 3.66E−95 | 6.37E−20 | 3.64E−59 |
| R48W | | | | | | | |
| PLCG1_ | K562-19 | Mutant | BBz | 6.25E−73 | 1.48E−51 | 2.36E−56 | 1 |
| R48W | | | | | | | |
| PLCG1_ | K562 | Mutant | 28z | 5.29E−61 | 0.00028489 | 1 | NA |
| S520F | | | | | | | |
| PLCG1_ | K562 | Mutant | BBz | 1 | 1 | 1 | NA |
| S520F | | | | | | | |
| PLCG1_ | K562-19 | Mutant | 28z | 2.05E−49 | 1.53E−58 | 1.03E−28 | 1 |
| S520F | | | | | | | |
| PLCG1_ | K562-19 | Mutant | BBz | 8.50E−43 | 4.31E−21 | 2.26E−31 | 1 |
| S520F | | | | | | | |
| PRKCB1 | K562 | Wild-type | 28z | 1 | 1 | 1 | NA |
| PRKCB1 | K562 | Wild-type | BBz | 1 | 1 | 1 | NA |
| PRKCB1 | K562-19 | Wild-type | 28z | 1 | 4.62E−07 | 0.00270522 | 1 |
| PRKCB1 | K562-19 | Wild-type | BBz | 0.421344 | 1.03E−08 | 0.00421344 | 1 |
| PRKCB1_ | K562 | Mutant | 28z | 1 | 1 | 1 | NA |
| D427N | | | | | | | |
| PRKCB1_ | K562 | Mutant | BBz | 1 | 1 | 1 | NA |
| D427N | | | | | | | |
| PRKCB1_ | K562-19 | Mutant | 28z | 9.48E−07 | 1.57E−23 | 1 | 1 |
| D427N | | | | | | | |
| PRKCB1_ | K562-19 | Mutant | BBz | 1 | 3.14E−16 | 1 | 1 |
| D427N | | | | | | | |
| PRKCB1_ | K562 | Mutant | 28z | 1 | 1 | 1 | NA |
| D630Y | | | | | | | |
| PRKCB1_ | K562 | Mutant | BBz | 1 | 1 | 1 | NA |
| D630Y | | | | | | | |
| PRKCB1_ | K562-19 | Mutant | 28z | 1 | 1.12E−05 | 1 | 1 |
| D630Y | | | | | | | |
| PRKCB1_ | K562-19 | Mutant | BBz | 1 | 1.46E−09 | 1 | 1 |
| D630Y | | | | | | | |
| RARA | K562 | Wild-type | 28z | 1 | 1 | 1 | NA |
| RARA | K562 | Wild-type | BBz | 1 | 1 | 1 | NA |
| RARA | K562-19 | Wild-type | 28z | 1.95E−24 | 4.17E−05 | 1 | 1 |
| RARA | K562-19 | Wild-type | BBz | 2.73E−21 | 1 | 1 | 1 |
| RARA_ | K562 | Mutant | 28z | 1 | 1 | 1 | NA |
| G206S | | | | | | | |
| RARA_ | K562 | Mutant | BBz | 1 | 1 | 1 | NA |
| G206S | | | | | | | |
| RARA_ | K562-19 | Mutant | 28z | 6.39E−21 | 0.01970262 | 1 | 1 |
| G206S | | | | | | | |
| RARA_ | K562-19 | Mutant | BBZ | 1.90E−25 | 1 | 1 | 1 |
| G206S | | | | | | | |
| RASGRP1 | K562 | Wild-type | 28z | 1.41E−30 | 0.00025616 | 0.00094563 | NA |
| RASGRP1 | K562 | Wild-type | BBz | 1 | 1 | 1 | NA |
| RASGRP1 | K562-19 | Wild-type | 28z | 3.73E−94 | 4.24E−92 | 2.39E−41 | 0.00095524 |
| RASGRP1 | K562-19 | Wild-type | BBz | 8.43E−70 | 1.05E−46 | 8.95E−71 | 1 |
| RASGRP1_ | K562 | Mutant | 28z | 1.12E−28 | 2.22E−15 | 2.92E−11 | NA |
| M261I | | | | | | | |
| RASGRP1_ | K562 | Mutant | BBz | 1 | 1 | 0.01560888 | NA |
| M261I | | | | | | | |
| RASGRP1_ | K562-19 | Mutant | 28z | 1.04E−120 | 1.28E−99 | 1.02E−71 | 2.97E−70 |
| M261I | | | | | | | |
| RASGRP1_ | K562-19 | Mutant | BBz | 3.57E−108 | 1.74E−60 | 5.51E−90 | 1 |
| M261I | | | | | | | |
| RHOA | K562 | Wild-type | 28z | 1 | 1 | 1 | NA |
| RHOA | K562 | Wild-type | BBz | 1 | 1 | 1 | NA |
| RHOA | K562-19 | Wild-type | 28z | 1 | 1 | 1 | 1 |
| RHOA | K562-19 | Wild-type | BBz | 1 | 1 | 1 | 1 |
| RHOA_ | K562 | Mutant | 28z | 1 | 1 | 1 | NA |
| C16R | | | | | | | |
| RHOA_ | K562 | Mutant | BBz | 1 | 1 | 1 | NA |
| C16R | | | | | | | |
| RHOA_ | K562-19 | Mutant | 28z | 1 | 1 | 1 | 1 |
| C16R | | | | | | | |
| RHOA_ | K562-19 | Mutant | BBz | 1 | 1 | 1 | 1 |
| C16R | | | | | | | |
| RHOA_ | K562 | Mutant | 28z | 1 | 1 | 1 | NA |
| G17V | | | | | | | |
| RHOA_ | K562 | Mutant | BBz | 1 | 1 | 1 | NA |
| G17V | | | | | | | |
| RHOA_ | K562-19 | Mutant | 28z | 1 | 1 | 1 | 1 |
| G17V | | | | | | | |
| RHOA_ | K562-19 | Mutant | BBz | 1 | 1 | 1 | 1 |
| G17V | | | | | | | |
| SELENOI- | K562 | Fusion | 28z | 1 | 4.02E−12 | 1 | NA |
| ABL1 | | | | | | | |
| SELENOI- | K562 | Fusion | BBz | 1 | 1 | 1 | NA |
| ABL1 | | | | | | | |
| SELENOI- | K562-19 | Fusion | 28z | 3.47E−29 | 2.02E−116 | 2.68E−06 | 1 |
| ABL1 | | | | | | | |
| SELENOI- | K562-19 | Fusion | BBz | 8.28E−15 | 1.52E−48 | 5.39E−07 | 1 |
| ABL1 | | | | | | | |
| SMARCB1 | K562 | Wild-type | 28z | 1 | 1 | 1 | NA |
| SMARCB1 | K562 | Wild-type | BBz | 1 | 1 | 1 | NA |
| SMARCB1 | K562-19 | Wild-type | 28z | 1 | 0.1989414 | 1 | 1 |
| SMARCB1 | K562-19 | Wild-type | BBz | 1 | 1 | 1 | 1 |
| SMARCB1_ | K562 | Mutant | 28z | 1 | 1 | 1 | NA |
| Q368X | | | | | | | |
| SMARCB1_ | K562 | Mutant | BBZ | 1 | 1 | 1 | NA |
| Q368X | | | | | | | |
| SMARCB1_ | K562-19 | Mutant | 28z | 0.761292 | 2.54E−06 | 1 | 1 |
| Q368X | | | | | | | |
| SMARCB1_ | K562-19 | Mutant | BBz | 1 | 1 | 1 | 1 |
| Q368X | | | | | | | |
| SPI1- | K562 | Fusion | 28z | 1 | 1 | 1 | NA |
| TCF7 | | | | | | | |
| SPI1- | K562 | Fusion | BBz | 1 | 1 | 1 | NA |
| TCF7 | | | | | | | |
| SPI1- | K562-19 | Fusion | 28z | 1 | 1 | 1 | 1 |
| TCF7 | | | | | | | |
| SPI1- | K562-19 | Fusion | BBz | 1 | 1 | 1 | 1 |
| TCF7 | | | | | | | |
| STAT3 | K562 | Wild-type | 28z | 1 | 1 | 1 | NA |
| STAT3 | K562 | Wild-type | BBz | 1 | 1 | 1 | NA |
| STAT3 | K562-19 | Wild-type | 28z | 0.643986 | 1 | 1 | 1 |
| STAT3 | K562-19 | Wild-type | BBz | 1 | 1 | 0.0140049 | 1 |
| STAT3_ | K562 | Mutant | 28z | 1 | 1 | 1 | NA |
| D661I | | | | | | | |
| STAT3_ | K562 | Mutant | BBz | 1 | 1 | 1 | NA |
| D661I | | | | | | | |
| STAT3_ | K562-19 | Mutant | 28z | 1.92E−08 | 0.253764 | 1.98E−06 | 1 |
| D661I | | | | | | | |
| STAT3_ | K562-19 | Mutant | BBz | 0.0306432 | 1 | 1.85E−06 | 1 |
| D661I | | | | | | | |
| STAT3_ | K562 | Mutant | 28z | 0.265734 | 1 | 1 | NA |
| G618R | | | | | | | |
| STAT3_ | K562 | Mutant | BBz | 1 | 1 | 1 | NA |
| G618R | | | | | | | |
| STAT3_ | K562-19 | Mutant | 28z | 1 | 1 | 0.137655 | 1 |
| G618R | | | | | | | |
| STAT3_ | K562-19 | Mutant | BBz | 0.00318402 | 1 | 0.00327978 | 1 |
| G618R | | | | | | | |
| STAT3_ | K562 | Mutant | 28z | 1 | 1 | 1 | NA |
| N647I | | | | | | | |
| STAT3_ | K562 | Mutant | BBz | 1 | 1 | 1 | NA |
| N647I | | | | | | | |
| STAT3_ | K562-19 | Mutant | 28z | 1 | 1 | 0.00270522 | 1 |
| N647I | | | | | | | |
| STAT3_ | K562-19 | Mutant | BBz | 0.0029925 | 1 | 0.2001384 | 1 |
| N647I | | | | | | | |
| STAT5B | K562 | Wild-type | 28z | 1 | 1 | 1 | NA |
| STAT5B | K562 | Wild-type | BBz | 1 | 1 | 1 | NA |
| STAT5B | K562-19 | Wild-type | 28z | 1 | 1 | 1 | 1 |
| STAT5B | K562-19 | Wild-type | BBz | 1 | 1 | 1 | 1 |
| STAT5B_ | K562 | Mutant | 28z | 1 | 1 | 1 | NA |
| T628S | | | | | | | |
| STAT5B_ | K562 | Mutant | BBz | 1 | 1 | 1 | NA |
| T628S | | | | | | | |
| STAT5B_ | K562-19 | Mutant | 28z | 1 | 1 | 1 | 1 |
| T628S | | | | | | | |
| STAT5B_ | K562-19 | Mutant | BBz | 1 | 1 | 1 | 1 |
| T628S | | | | | | | |
| STAT5B_ | K562 | Mutant | 28z | 1 | 1 | 1 | NA |
| Y665F | | | | | | | |
| STAT5B_ | K562 | Mutant | BBz | 1 | 1 | 1 | NA |
| Y665F | | | | | | | |
| STAT5B_ | K562-19 | Mutant | 28z | 1 | 1 | 1 | 1 |
| Y665F | | | | | | | |
| STAT5B_ | K562-19 | Mutant | BBz | - | 1 | 1 | 1 |
| Y665F | | | | | | | |
| TNFRSF1B | K562 | Wild-type | 28z | 1 | 1 | 1 | NA |
| TNFRSF1B | K562 | Wild-type | BBz | 1 | 0.0464436 | 1 | NA |
| TNFRSF1B | K562-19 | Wild-type | 28z | 0.00191999 | 6.42E−24 | 1 | 1 |
| TNFRSF1B | K562-19 | Wild-type | BBz | 1 | 6.89E−08 | 1 | 1 |
| TNFRSF1B_ | K562 | Mutant | 28z | 1 | 2.04E−05 | 1 | NA |
| G256C | | | | | | | |
| TNFRSF1B_ | K562 | Mutant | BBz | 1 | 8.16E−18 | 1 | NA |
| G256C | | | | | | | |
| TNFRSF1B_ | K562-19 | Mutant | 28z | 1 | 1.45E−42 | 1 | 1 |
| G256C | | | | | | | |
| TNFRSF1B_ | K562-19 | Mutant | BBz | 1 | 2.80E−21 | 1 | 1 |
| G256C | | | | | | | |
| TNFRSF1B_ | K562 | Mutant | 28z | 1 | 2.20E−23 | 1 | NA |
| T377I | | | | | | | |
| TNFRSF1B_ | K562 | Mutant | BBz | 1 | 2.05E−36 | 1 | NA |
| T377I | | | | | | | |
| TNFRSF1B_ | K562-19 | Mutant | 28z | 1 | 1.01E−59 | 1 | 1 |
| T377I | | | | | | | |
| TNFRSF1B_ | K562-19 | Mutant | BBz | 1 | 4.72E−31 | 1 | 1 |
| T377I | | | | | | | |
| TP53 | K562 | Wild-type | 28z | 1 | 1 | 1 | NA |
| TP53 | K562 | Wild-type | BBz | 1 | 1 | 1 | NA |
| TP53 | K562-19 | Wild-type | 28z | 1 | 3.42E−07 | 1 | 1 |
| TP53 | K562-19 | Wild-type | BBz | 1 | 0.914508 | 1 | 1 |
| TP53_ | K562 | Mutant | 28z | 1 | 1 | 1 | NA |
| R273P | | | | | | | |
| TP53_ | K562 | Mutant | BBz | 1 | 1 | 1 | NA |
| R273P | | | | | | | |
| TP53_ | K562-19 | Mutant | 28z | 1 | 1 | 1 | 1 |
| R273P | | | | | | | |
| TP53_ | K562-19 | Mutant | BBz | 1 | 1 | 1 | 1 |
| R273P | | | | | | | |
| VAV1 | K562 | Wild-type | 28z | 3.33E−151 | 1 | 1 | NA |
| VAV1 | K562 | Wild-type | BBz | | 1 | 1 | NA |
| VAV1 | K562-19 | Wild-type | 28z | 0.00821142 | 1 | 0.00270522 | 1 |
| VAV1 | K562-19 | Wild-type | BBz | 3.93E−13 | 0.00093605 | 5.72E−08 | 1 |
| VAV1_ | K562 | Mutant | 28z | 1.67E−07 | 1 | 1 | NA |
| D797H | | | | | | | |
| VAV1_ | K562 | Mutant | BBZ | 1 | 1 | 1 | NA |
| D797H | | | | | | | |
| VAV1_ | K562-19 | Mutant | 28z | 2.30E−06 | 1 | 1.98E−06 | 1 |
| D797H | | | | | | | |
| VAV1_ | K562-19 | Mutant | BBz | 8.50E−06 | 7.95E−06 | 3.40E−06 | 1 |
| D797H | | | | | | | |
| VAV1_ | K562 | Mutant | 28z | 5.94E−05 | 1 | 1 | NA |
| R798Q | | | | | | | |
| VAV1_ | K562 | Mutant | BBz | 1 | 1 | 1 | NA |
| R798Q | | | | | | | |
| VAV1_ | K562-19 | Mutant | 28z | 0.00821142 | 1 | 0.00057935 | 1 |
| R798Q | | | | | | | |
| VAV1_ | K562-19 | Mutant | BBz | 1.00E−09 | 2.82E−09 | 5.72E−08 | 1 |
| R798Q | | | | | | | |
| VAV1- | K562 | Fusion | 28z | 1.19E−06 | 1 | 1 | NA |
| GSS | | | | | | | |
| VAV1- | K562 | Fusion | BBz | 1 | 1 | 1 | NA |
| GSS | | | | | | | |
| VAV1- | K562-19 | Fusion | 28z | 0.2353302 | 1 | 1 | 1 |
| GSS | | | | | | | |
| VAV1- | K562-19 | Fusion | BBz | 2.94E−13 | 2.82E−09 | 2.87E−07 | 1 |
| GSS | | | | | | | |
| | construct | ctrl_pval_pd1 | wt_pval_nfat | wt_pval_nfkb | wt_pval_ap1 | wt_pval_il2 | wt_pval_pd1 |
| | BCL6 | NA | NA | NA | NA | NA | NA |
| | BCL6 | 1 | NA | NA | NA | NA | NA |
| | BCL6 | NA | NA | NA | NA | NA | NA |
| | BCL6 | 1.54E−27 | NA | NA | NA | NA | NA |
| | BCL6_ | NA | 1 | 1 | 1 | NA | NA |
| | S647R | | | | | | |
| | BCL6_ | 1 | 1 | 1 | 1 | NA | 1 |
| | S647R | | | | | | |
| | BCL6_ | NA | 5.00E−05 | 1 | 0.01982232 | 1 | NA |
| | S647R | | | | | | |
| | BCL6_ | 1 | 0.0871416 | 1 | 1 | 1 | 4.43E−19 |
| | S647R | | | | | | |
| | BRAF | NA | NA | NA | NA | NA | NA |
| | BRAF | 1 | NA | NA | NA | NA | NA |
| | BRAF | NA | NA | NA | NA | NA | NA |
| | BRAF | 1 | NA | NA | NA | NA | NA |
| | BRAF_ | NA | 1 | 1 | 1 | NA | NA |
| | D594N | | | | | | |
| | BRAF_ | 1 | 1 | 1 | 1 | NA | 1 |
| | D594N | | | | | | |
| | BRAF_ | NA | 1 | 5.41E−06 | 1 | 1 | NA |
| | D594N | | | | | | |
| | BRAF_ | 1 | 1 | 1 | 1 | 1 | 1 |
| | D594N | | | | | | |
| | BRAF_ | NA | 4.64E−25 | 2.24E−40 | 1.30E−32 | NA | NA |
| | G469A | | | | | | |
| | BRAF_ | 0.0316008 | 0.1201788 | 2.61E−37 | 1.47E−24 | NA | 0.00049316 |
| | G469A | | | | | | |
| | BRAF_ | NA | 4.55E−109 | 2.44E−49 | 8.07E−22 | 9.08E−98 | NA |
| | G469A | | | | | | |
| | BRAF_ | 1 | 2.12E−72 | 6.97E−38 | 4.07E−39 | 1.84E−05 | 1 |
| | G469A | | | | | | |
| | BRAF_ | NA | 1 | 1 | 1 | NA | NA |
| | G469R | | | | | | |
| | BRAF_ | 1 | 1 | 1 | 1 | NA | 1 |
| | G469R | | | | | | |
| | BRAF_ | NA | 1 | 5.39E−17 | 0.00189365 | 1 | NA |
| | G469R | | | | | | |
| | BRAF_ | 1 | 0.0073017 | 0.01558494 | 2.54E−14 | 1 | 1 |
| | G469R | | | | | | |
| | CARD11 | NA | NA | NA | NA | NA | NA |
| | CARD11 | 1 | NA | NA | NA | NA | NA |
| | CARD11 | NA | NA | NA | NA | NA | NA |
| | CARD11 | 0.588924 | NA | NA | NA | NA | NA |
| | CARD11_ | NA | 1 | 7.47E−69 | 1 | NA | NA |
| | D357N | | | | | | |
| | CARD11_ | 1 | 1 | 5.05E−33 | 1 | NA | 1 |
| | D357N | | | | | | |
| | CARD11_ | NA | 1 | 1.60E−12 | 3.14E−21 | 3.81E−60 | NA |
| | D357N | | | | | | |
| | CARD11_ | 1.69E−15 | 1 | 6.87E−23 | 1.46E−22 | 1 | 0.0378252 |
| | D357N | | | | | | |
| | CARD11_ | NA | 1 | 1 | 1 | NA | NA |
| | E634K: | | | | | | |
| | S655C | | | | | | |
| | CARD11_ | 1 | 1 | 1 | 1 | NA | 1 |
| | E634K: | | | | | | |
| | S655C | | | | | | |
| | CARD11_ | NA | 1 | 1 | 1 | 1 | NA |
| | E634K: | | | | | | |
| | S655C | | | | | | |
| | CARD11_ | 6.10E−06 | 1 | 3.50E−05 | 0.0892962 | 1 | 1 |
| | E634K: | | | | | | |
| | S655C | | | | | | |
| | CARD11_ | NA | 1 | 1 | 1 | NA | NA |
| | S615F | | | | | | |
| | CARD11_ | 1 | 1 | 1 | 1 | NA | 1 |
| | S615F | | | | | | |
| | CARD11_ | NA | 1 | 2.35E−13 | 6.42E−24 | 2.18E−10 | NA |
| | S615F | | | | | | |
| | CARD11_ | 2.23E−10 | 1 | 3.23E−20 | 1.59E−31 | 1 | 1 |
| | S615F | | | | | | |
| | CARD11_ | NA | 1 | 1.06E−57 | 1 | NA | NA |
| | Y361C | | | | | | |
| | CARD11_ | 1 | 1 | 1.83E−13 | 1 | NA | 1 |
| | Y361C | | | | | | |
| | CARD11_ | NA | 1 | 1.04E−15 | 4.69E−37 | 3.33E−65 | NA |
| | Y361C | | | | | | |
| | CARD11_ | 5.43E−16 | 1 | 4.33E−36 | 1.44E−70 | 0.0287574 | 0.02271906 |
| | Y361C | | | | | | |
| | CARD11- | NA | NA | NA | NA | NA | NA |
| | PIK3R3 | | | | | | |
| | CARD11- | 1 | NA | NA | NA | NA | NA |
| | PIK3R3 | | | | | | |
| | CARD11- | NA | NA | NA | NA | NA | NA |
| | PIK3R3 | | | | | | |
| | CARD11- | 8.04E−25 | NA | NA | NA | NA | NA |
| | PIK3R3 | | | | | | |
| | CARMIL2_ | NA | NA | NA | NA | NA | NA |
| | Q575E | | | | | | |
| | CARMIL2_ | 1 | NA | NA | NA | NA | NA |
| | Q575E | | | | | | |
| | CARMIL2_ | NA | NA | NA | NA | NA | NA |
| | Q575E | | | | | | |
| | CARMIL2_ | 1 | NA | NA | NA | NA | NA |
| | Q575E | | | | | | |
| | CCND3 | NA | NA | NA | NA | NA | NA |
| | CCND3 | 1 | NA | NA | NA | NA | NA |
| | CCND3 | NA | NA | NA | NA | NA | NA |
| | CCND3 | 1.37E−19 | NA | NA | NA | NA | NA |
| | CCND3_ | NA | 1 | 1 | 1 | NA | NA |
| | P284S | | | | | | |
| | CCND3_ | 1 | 1 | 1 | 1 | NA | 1 |
| | P284S | | | | | | |
| | CCND3_ | NA | 1 | 1 | 1 | 1 | NA |
| | P284S | | | | | | |
| | CCND3_ | 1.81E−26 | 1 | 1 | 1 | 1 | 1 |
| | P284S | | | | | | |
| | CD28 | NA | NA | NA | NA | NA | NA |
| | CD28 | 1 | NA | NA | NA | NA | NA |
| | CD28 | NA | NA | NA | NA | NA | NA |
| | CD28 | 1 | NA | NA | NA | NA | NA |
| | CD28_ | NA | 1 | 1 | 1 | NA | NA |
| | F51I | | | | | | |
| | CD28_ | 1 | 1 | 1 | 1 | NA | 1 |
| | F51I | | | | | | |
| | CD28_ | NA | 1 | 1 | 1 | 1 | NA |
| | F51I | | | | | | |
| | CD28_ | 1 | 1 | 1 | 1 | 1 | 1 |
| | F51I | | | | | | |
| | CD28_ | NA | 1 | 1 | 1 | NA | NA |
| | F51V | | | | | | |
| | CD28_ | 1 | 1 | 1 | 1 | NA | 1 |
| | F51V | | | | | | |
| | CD28_ | NA | 1 | 1 | 1 | 1 | NA |
| | F51V | | | | | | |
| | CD28_ | 1 | 1 | 1 | 1 | 1 | 1 |
| | F51V | | | | | | |
| | CD28_ | NA | 1 | 1 | 1 | NA | NA |
| | Q77P | | | | | | |
| | CD28_ | 1 | 1 | 1 | 1 | NA | 1 |
| | Q77P | | | | | | |
| | CD28_ | NA | 1 | 1 | 1 | 1 | NA |
| | Q77P | | | | | | |
| | CD28_ | 1 | 1 | 1 | 1 | 1 | 1 |
| | Q77P | | | | | | |
| | CD28_ | NA | 1 | 1 | 1 | NA | NA |
| | T195P | | | | | | |
| | CD28_ | 1 | 1 | 1 | 1 | NA | 1 |
| | T195P | | | | | | |
| | CD28_ | NA | 1 | 1 | 1 | 1 | NA |
| | T195P | | | | | | |
| | CD28_ | 0.495558 | 1 | 1 | 0.655956 | 1 | 1 |
| | T195P | | | | | | |
| | CD28- | NA | NA | NA | NA | NA | NA |
| | CTLA4 | | | | | | |
| | CD28- | 1 | NA | NA | NA | NA | NA |
| | CTLA4 | | | | | | |
| | CD28- | NA | NA | NA | NA | NA | NA |
| | CTLA4 | | | | | | |
| | CD28- | 1 | NA | NA | NA | NA | NA |
| | CTLA4 | | | | | | |
| | CD3E | NA | NA | NA | NA | NA | NA |
| | CD3E | 1 | NA | NA | NA | NA | NA |
| | CD3E | NA | NA | NA | NA | NA | NA |
| | CD3E | 1 | NA | NA | NA | NA | NA |
| | CD3E_ | NA | 1 | 1 | 1 | NA | NA |
| | S41C | | | | | | |
| | CD3E_ | 1 | 1 | 1 | 1 | NA | 1 |
| | S41C | | | | | | |
| | CD3E_ | NA | 1 | 1 | 1 | 1 | NA |
| | S41C | | | | | | |
| | CD3E_ | 0.737352 | 1 | 1 | 1 | 1 | 1 |
| | S41C | | | | | | |
| | CD53 | NA | NA | NA | NA | NA | NA |
| | CD53 | 1 | NA | NA | NA | NA | NA |
| | CD53 | NA | NA | NA | NA | NA | NA |
| | CD53 | 1 | NA | NA | NA | NA | NA |
| | CSNK1A1 | NA | NA | NA | NA | NA | NA |
| | CSNK1A1 | 1 | NA | NA | NA | NA | NA |
| | CSNK1A1 | NA | NA | NA | NA | NA | NA |
| | CSNK1A1 | 1 | NA | NA | NA | NA | NA |
| | CSNK1A1_ | NA | 1 | 1 | 1 | NA | NA |
| | S27C | | | | | | |
| | CSNK1A1_ | 1 | 1 | 1 | 1 | NA | 1 |
| | S27C | | | | | | |
| | CSNK1A1_ | NA | 1 | 1 | 1 | 1 | NA |
| | S27C | | | | | | |
| | CSNK1A1_ | 1 | 1 | 1 | 1 | 1 | 1 |
| | S27C | | | | | | |
| | CSNK2B | NA | NA | NA | NA | NA | NA |
| | CSNK2B | 1 | NA | NA | NA | NA | NA |
| | CSNK2B | NA | NA | NA | NA | NA | NA |
| | CSNK2B | 1 | NA | NA | NA | NA | NA |
| | CSNK2B_ | NA | 1 | 1 | 1 | NA | NA |
| | Q182X | | | | | | |
| | CSNK2B_ | 1 | 1 | 1 | 1 | NA | 1 |
| | Q182X | | | | | | |
| | CSNK2B_ | NA | 1 | 1 | 1 | 1 | NA |
| | Q182X | | | | | | |
| | CSNK2B_ | 0.0866628 | 1 | 1 | 1 | 1 | 1 |
| | Q182X | | | | | | |
| | DGKA | NA | NA | NA | NA | NA | NA |
| | DGKA | 1 | NA | NA | NA | NA | NA |
| | DGKA | NA | NA | NA | NA | NA | NA |
| | DGKA | 1 | NA | NA | NA | NA | NA |
| | DGKZ | NA | NA | NA | NA | NA | NA |
| | DGKZ | 1 | NA | NA | NA | NA | NA |
| | DGKZ | NA | NA | NA | NA | NA | NA |
| | DGKZ | 1.32E−21 | NA | NA | NA | NA | NA |
| | ECSIT | NA | NA | NA | NA | NA | NA |
| | ECSIT | 1 | NA | NA | NA | NA | NA |
| | ECSIT | NA | NA | NA | NA | NA | NA |
| | ECSIT | 1 | NA | NA | NA | NA | NA |
| | ECSIT_ | NA | 1 | 1 | 1 | NA | NA |
| | V140A | | | | | | |
| | ECSIT_ | 1 | 1 | 1 | 1 | NA | 1 |
| | V140A | | | | | | |
| | ECSIT_ | NA | 1 | 1 | 1 | 1 | NA |
| | V140A | | | | | | |
| | ECSIT_ | 1 | 1 | 1 | 1 | 1 | 1 |
| | V140A | | | | | | |
| | EIFS1 | NA | NA | NA | NA | NA | NA |
| | EIFS1 | 1 | NA | NA | NA | NA | NA |
| | EIFS1 | NA | NA | NA | NA | NA | NA |
| | EIFS1 | 0.0940842 | NA | NA | NA | NA | NA |
| | EIFS1_ | NA | 1 | 1 | 1 | NA | NA |
| | R89I | | | | | | |
| | EIFS1_ | 1 | 1 | 1 | 1 | NA | 1 |
| | R89I | | | | | | |
| | EIFS1_ | NA | 1 | 1 | 1 | 1 | NA |
| | R89I | | | | | | |
| | EIFS1_ | 0.20995 | 1 | 1 | 1 | 1 | 1 |
| | R89I | 38 | | | | | |
| | FYN | NA | NA | NA | NA | NA | NA |
| | FYN | 1 | NA | NA | NA | NA | NA |
| | FYN | NA | NA | NA | NA | NA | NA |
| | FYN | 4.91E−05 | NA | NA | NA | NA | NA |
| | FYN_ | NA | 1 | 1 | 1 | NA | NA |
| | R86G | | | | | | |
| | FYN_ | 1 | 1 | 1 | 1 | NA | 1 |
| | R86G | | | | | | |
| | FYN_ | NA | 0.00011491 | 0.00017093 | 1 | 1 | NA |
| | R86G | | | | | | |
| | FYN_ | 0.00234612 | 1 | 1 | 1 | 1 | 1 |
| | R86G | | | | | | |
| | FYN_ | NA | 1 | 1 | 1 | NA | NA |
| | Y531H | | | | | | |
| | FYN_ | 1 | 1 | 1 | 1 | NA | 1 |
| | Y531H | | | | | | |
| | FYN_ | NA | 1 | 1 | 1 | 1 | NA |
| | Y531H | | | | | | |
| | FYN_ | 1.22E−06 | 1 | 1 | 1 | 1 | 1 |
| | Y531H | | | | | | |
| | GATA3 | NA | NA | NA | NA | NA | NA |
| | GATA3 | 1 | NA | NA | NA | NA | NA |
| | GATA3 | NA | NA | NA | NA | NA | NA |
| | GATA3 | 1.29E−11 | NA | NA | NA | NA | NA |
| | GATA3_ | NA | 1 | 1 | 1 | NA | NA |
| | Y63X | | | | | | |
| | GATA3_ | 1 | 1 | 1 | 1 | NA | 1 |
| | Y63X | | | | | | |
| | GATA3_ | NA | 0.00022312 | 1.55E−08 | 1 | 1 | NA |
| | Y63X | | | | | | |
| | GATA3_ | 1 | 1 | 1 | 0.363888 | 1 | 0.0284886 |
| | Y63X | | | | | | |
| | GNAQ | NA | NA | NA | NA | NA | NA |
| | GNAQ | 1 | NA | NA | NA | NA | NA |
| | GNAQ | NA | NA | NA | NA | NA | NA |
| | GNAQ | 1 | NA | NA | NA | NA | NA |
| | GNAQ_ | NA | 1 | 1 | 1 | NA | NA |
| | T96S | | | | | | |
| | GNAQ_ | 1 | 1 | 1 | 1 | NA | 1 |
| | T96S | | | | | | |
| | GNAQ_ | NA | 1 | 1 | 1 | 1 | NA |
| | T96S | | | | | | |
| | GNAQ_ | 1 | 1 | 1 | 1 | 1 | 1 |
| | T96S | | | | | | |
| | ICOS- | NA | NA | NA | NA | NA | NA |
| | CD28 | | | | | | |
| | ICOS- | 1 | NA | NA | NA | NA | NA |
| | CD28 | | | | | | |
| | ICOS- | NA | NA | NA | NA | NA | NA |
| | CD28 | | | | | | |
| | ICOS- | 1 | NA | NA | NA | NA | NA |
| | CD28 | | | | | | |
| | IRF4 | NA | NA | NA | NA | NA | NA |
| | IRF4 | 1 | NA | NA | NA | NA | NA |
| | IRF4 | NA | NA | NA | NA | NA | NA |
| | IRF4 | 7.06E−28 | NA | NA | NA | NA | NA |
| | IRF4_ | NA | 1 | 1 | 1 | NA | NA |
| | K59R | | | | | | |
| | IRF4_ | 1 | 1 | 1 | 1 | NA | 1 |
| | K59R | | | | | | |
| | IRF4_ | NA | 1 | 1.82E−10 | 1.02E−09 | 1 | NA |
| | K59R | | | | | | |
| | IRF4_ | 2.51E−07 | 1 | 1 | 1 | 1 | 0.00313614 |
| | K59R | | | | | | |
| | ITGB2 | NA | NA | NA | NA | NA | NA |
| | ITGB2 | 1 | NA | NA | NA | NA | NA |
| | ITGB2 | NA | NA | NA | NA | NA | NA |
| | ITGB2 | 1 | NA | NA | NA | NA | NA |
| | ITGB2_ | NA | 1 | 1 | 1 | NA | NA |
| | E234K | | | | | | |
| | ITGB2_ | 1 | 1 | 1 | 1 | NA | 1 |
| | E234K | | | | | | |
| | ITGB2_ | NA | 1 | 1 | 1 | 1 | NA |
| | E234K | | | | | | |
| | ITGB2_ | 1 | 1 | 1 | 1 | 1 | 1 |
| | E234K | | | | | | |
| | ITK- | NA | NA | NA | NA | NA | NA |
| | FER | | | | | | |
| | ITK- | 1 | NA | NA | NA | NA | NA |
| | FER | | | | | | |
| | ITK- | NA | NA | NA | NA | NA | NA |
| | FER | | | | | | |
| | ITK- | 1 | NA | NA | NA | NA | NA |
| | FER | | | | | | |
| | ITK- | NA | NA | NA | NA | NA | NA |
| | SYK | | | | | | |
| | ITK- | 1 | NA | NA | NA | NA | NA |
| | SYK | | | | | | |
| | ITK- | NA | NA | NA | NA | NA | NA |
| | SYK | | | | | | |
| | ITK- | 1 | NA | NA | NA | NA | NA |
| | SYK | | | | | | |
| | JAK1_ | NA | NA | NA | NA | NA | NA |
| | G1097A | | | | | | |
| | JAK1_ | 1 | NA | NA | NA | NA | NA |
| | G1097A | | | | | | |
| | JAK1_ | NA | NA | NA | NA | NA | NA |
| | G1097A | | | | | | |
| | JAK1_ | 1 | NA | NA | NA | NA | NA |
| | G1097A | | | | | | |
| | JAK3 | NA | NA | NA | NA | NA | NA |
| | JAK3 | 1 | NA | NA | NA | NA | NA |
| | JAK3 | NA | NA | NA | NA | NA | NA |
| | JAK3 | 0.390222 | NA | NA | NA | NA | NA |
| | JAK3_ | NA | 1 | 1 | 1 | NA | NA |
| | A573V | | | | | | |
| | JAK3_ | 1 | 1 | 1 | 1 | NA | 1 |
| | A573V | | | | | | |
| | JAK3_ | NA | 1 | 1 | 1 | 1 | NA |
| | A573V | | | | | | |
| | JAK3_ | 1 | 1 | 1 | 1 | 1 | 1 |
| | A573V | | | | | | |
| | JUNB | NA | NA | NA | NA | NA | NA |
| | JUNB | 1 | NA | NA | NA | NA | NA |
| | JUNB | NA | NA | NA | NA | NA | NA |
| | JUNB | 6.99E−10 | NA | NA | NA | NA | NA |
| | JUNB_ | NA | 1 | 1 | 1 | NA | NA |
| | A282V | | | | | | |
| | JUNB_ | 1 | 1 | 1 | 1 | NA | 1 |
| | A282V | | | | | | |
| | JUNB_ | NA | 5.63E−10 | 1 | 0.0734958 | 1 | NA |
| | A282V | | | | | | |
| | JUNB_ | 7.45E−10 | 1 | 1 | 1 | 1 | 1 |
| | A282V | | | | | | |
| | KCNQ1 | NA | NA | NA | NA | NA | NA |
| | KCNQ1 | 1 | NA | NA | NA | NA | NA |
| | KCNQ1 | NA | NA | NA | NA | NA | NA |
| | KCNQ1 | 3.97E−05 | NA | NA | NA | NA | NA |
| | KCNQ1_ | NA | 1 | 1 | 1 | NA | NA |
| | R583C | | | | | | |
| | KCNQ1_ | 1 | 1 | 1 | 1 | NA | 1 |
| | R583C | | | | | | |
| | KCNQ1_ | NA | 1 | 1 | 1 | 1 | NA |
| | R583C | | | | | | |
| | KCNQ1_ | 0.0464436 | 1 | 1 | 1 | 1 | 1 |
| | R583C | | | | | | |
| | MSC | NA | NA | NA | NA | NA | NA |
| | MSC | 1 | NA | NA | NA | NA | NA |
| | MSC | NA | NA | NA | NA | NA | NA |
| | MSC | 8.74E−11 | NA | NA | NA | NA | NA |
| | MSC_ | NA | 1 | 1 | 1 | NA | NA |
| | E116K | | | | | | |
| | MSC_ | 1 | 1 | 0.378252 | 1 | NA | 1 |
| | E116K | | | | | | |
| | MSC_ | NA | 1 | 1.61E−28 | 0.00150822 | 1 | NA |
| | E116K | | | | | | |
| | MSC_ | 6.80E−14 | 1 | 7.68E−14 | 1 | 1 | 9.38E−26 |
| | E116K | | | | | | |
| | MYCN | NA | NA | NA | NA | NA | NA |
| | MYCN | 1 | NA | NA | NA | NA | NA |
| | MYCN | NA | NA | NA | NA | NA | NA |
| | MYCN | 0.00071102 | NA | NA | NA | NA | NA |
| | MYCN_ | NA | 1 | 1 | 1 | NA | NA |
| | P44L | | | | | | |
| | MYCN_ | 1 | 1 | 1 | 1 | NA | 1 |
| | P44L | | | | | | |
| | MYCN_ | NA | 1 | 1 | 1 | 1 | NA |
| | P44L | | | | | | |
| | MYCN_ | 4.38E−07 | 1 | 1 | 1 | 1 | 1 |
| | P44L | | | | | | |
| | NFKB1 | NA | NA | NA | NA | NA | NA |
| | NFKB1 | 1 | NA | NA | NA | NA | NA |
| | NFKB1 | NA | NA | NA | NA | NA | NA |
| | NFKB1 | 1 | NA | NA | NA | NA | NA |
| | NFKB1_ | NA | 1 | 1 | 1 | NA | NA |
| | H67Y | | | | | | |
| | NFKB1_ | 1 | 1 | 1 | 1 | NA | 1 |
| | H67Y | | | | | | |
| | NFKB1_ | NA | 1 | 1 | 1 | 1 | NA |
| | H67Y | | | | | | |
| | NFKB1_ | 0.00032319 | 1 | 1 | 1 | 1 | 1 |
| | H67Y | | | | | | |
| | NFKB2 | NA | NA | NA | NA | NA | NA |
| | NFKB2 | 1 | NA | NA | NA | NA | NA |
| | NFKB2 | NA | NA | NA | NA | NA | NA |
| | NFKB2 | 1 | NA | NA | NA | NA | NA |
| | NFKB2_ | NA | 1 | 1 | 1 | NA | NA |
| | K656X | | | | | | |
| | NFKB2_ | 1 | 1 | 0.01869714 | 1 | NA | 1 |
| | K656X | | | | | | |
| | NFKB2_ | NA | 1 | 4.29E−05 | 1 | 1 | NA |
| | K656X | | | | | | |
| | NFKB2_ | 4.19E−05 | 1 | 0.00471618 | 0.0462042 | 1 | 1 |
| | K656X | | | | | | |
| | NPM- | NA | NA | NA | NA | NA | NA |
| | ALK | | | | | | |
| | NPM- | 1 | NA | NA | NA | NA | NA |
| | ALK | | | | | | |
| | NPM- | NA | NA | NA | NA | NA | NA |
| | ALK | | | | | | |
| | NPM- | 1.85E−10 | NA | NA | NA | NA | NA |
| | ALK | | | | | | |
| | NPM- | NA | NA | NA | NA | NA | NA |
| | TYK | | | | | | |
| | NPM- | 1 | NA | NA | NA | NA | NA |
| | TYK | | | | | | |
| | NPM- | NA | NA | NA | NA | NA | NA |
| | TYK | | | | | | |
| | NPM- | 0.2360484 | NA | NA | NA | NA | NA |
| | TYK | | | | | | |
| | NRAS | NA | NA | NA | NA | NA | NA |
| | NRAS | 1 | NA | NA | NA | NA | NA |
| | NRAS | NA | NA | NA | NA | NA | NA |
| | NRAS | 0.01656648 | NA | NA | NA | NA | NA |
| | NRAS_ | NA | 1 | 1 | 1 | NA | NA |
| | G13D | | | | | | |
| | NRAS_ | 1 | 1 | 1 | 1 | NA | 1 |
| | G13D | | | | | | |
| | NRAS_ | NA | 0.225036 | 0.01838592 | 1 | 1 | NA |
| | G13D | | | | | | |
| | NRAS_ | 1.53E−05 | 1 | 1 | 1 | 1 | 1 |
| | G13D | | | | | | |
| | NRAS_ | NA | 1 | 1 | 1 | NA | NA |
| | Q61H | | | | | | |
| | NRAS_ | 1 | 1 | 1 | 1 | NA | 1 |
| | Q61H | | | | | | |
| | NRAS_ | NA | 0.00057456 | 6.68E−06 | 0.363888 | 1 | NA |
| | Q61H | | | | | | |
| | NRAS_ | 1.74E−07 | 1 | 1 | 1 | 1 | 1 |
| | Q61H | | | | | | |
| | PDCD1 | NA | NA | NA | NA | NA | NA |
| | PDCD1 | 7.16E−273 | NA | NA | NA | NA | NA |
| | PDCD1 | NA | NA | NA | NA | NA | NA |
| | PDCD1 | 1.12E−264 | NA | NA | NA | NA | NA |
| | PDCD1_ | NA | 1 | 1 | 1 | NA | NA |
| | R231X | | | | | | |
| | PDCD1_ | 2.00E−272 | 1 | 1 | 1 | NA | 1 |
| | R231X | | | | | | |
| | PDCD1_ | NA | 1 | 1 | 1 | 1 | NA |
| | R231X | | | | | | |
| | PDCD1_ | 1.95E−264 | 1 | 1 | 1 | 1 | 1 |
| | R231X | | | | | | |
| | PLCG1 | NA | NA | NA | NA | NA | NA |
| | PLCG1 | 1 | NA | NA | NA | NA | NA |
| | PLCG1 | NA | NA | NA | NA | NA | NA |
| | PLCG1 | 0.0292068 | NA | NA | NA | NA | NA |
| | PLCG1_ | NA | 3.42E−45 | 9.29E−13 | 1 | NA | NA |
| | D1165H | | | | | | |
| | PLCG1_ | 1 | 1 | 1 | 1 | NA | 1 |
| | D1165H | | | | | | |
| | PLCG1_ | NA | 4.14E−35 | 2.51E−53 | 8.07E−22 | 1 | NA |
| | D1165H | | | | | | |
| | PLCG1_ | 1 | 5.10E−43 | 1.71E−20 | 1.16E−43 | 1 | 0.1264032 |
| | D1165H | | | | | | |
| | PLCG1_ | NA | 1 | 0.517104 | 1 | NA | NA |
| | E1163K | | | | | | |
| | PLCG1_ | 1 | 1 | 1 | 1 | NA | 1 |
| | E1163K | | | | | | |
| | PLCG1_ | NA | 6.37E−29 | 1.10E−49 | 1.48E−12 | 0.00015164 | NA |
| | E1163K | | | | | | |
| | PLCG1_ | 1 | 3.71E−19 | 1.22E−12 | 1.15E−17 | 1 | 1 |
| | E1163K | | | | | | |
| | PLCG1_ | NA | 1 | 1 | 1 | NA | NA |
| | E47K | | | | | | |
| | PLCG1_ | 1 | 1 | 1 | 1 | NA | 1 |
| | E47K | | | | | | |
| | PLCG1_ | NA | 2.78E−16 | 5.41E−31 | 0.01668618 | 0.39078 | NA |
| | E47K | | | | | | |
| | PLCG1_ | 1 | 0.00998298 | 0.00232936 | 0.758898 | 1 | 0.00845082 |
| | E47K | | | | | | |
| | PLCG1_ | NA | 0.049077 | 0.00052668 | 1 | NA | NA |
| | R48W | | | | | | |
| | PLCG1_ | 1 | 1 | 1 | 1 | NA | 1 |
| | R48W | | | | | | |
| | PLCG1_ | NA | 3.11E−48 | 1.20E−56 | 6.06E−10 | 1.65E−33 | NA |
| | R48W | | | | | | |
| | PLCG1_ | 7.40E−22 | 2.61E−32 | 9.43E−22 | 9.15E−29 | 1 | 4.31E−23 |
| | R48W | | | | | | |
| | PLCG1_ | NA | 3.50E−32 | 0.2348514 | 1 | NA | NA |
| | S520F | | | | | | |
| | PLCG1_ | 1 | 1 | 1 | 1 | NA | 1 |
| | S520F | | | | | | |
| | PLCG1_ | NA | 7.83E−24 | 7.76E−31 | 3.09E−15 | 1 | NA |
| | S520F | | | | | | |
| | PLCG1_ | 1 | 4.45E−14 | 3.50E−05 | 1.98E−13 | 1 | 1 |
| | S520F | | | | | | |
| | PRKCB1 | NA | NA | NA | NA | NA | NA |
| | PRKCB1 | 1 | NA | NA | NA | NA | NA |
| | PRKCB1 | NA | NA | NA | NA | NA | NA |
| | PRKCB1 | 8.02E−06 | NA | NA | NA | NA | NA |
| | PRKCB1_ | NA | 1 | 1 | 1 | NA | NA |
| | D427N | | | | | | |
| | PRKCB1_ | 1 | 1 | 1 | 1 | NA | 1 |
| | D427N | | | | | | |
| | PRKCB1_ | NA | 0.00080917 | 0.703836 | 0.02355696 | 1 | NA |
| | D427N | | | | | | |
| | PRKCB1_ | 3.30E−16 | 0.00089057 | 1 | 0.0280098 | 1 | 1 |
| | D427N | | | | | | |
| | PRKCB1_ | NA | 1 | 1 | 1 | NA | NA |
| | D630Y | | | | | | |
| | PRKCB1_ | 1 | 1 | 1 | 1 | NA | 1 |
| | D630Y | | | | | | |
| | PRKCB1_ | NA | 1 | 1 | 1 | 1 | NA |
| | D630Y | | | | | | |
| | PRKCB1_ | 2.00E−05 | 1 | 1 | 1 | 1 | 1 |
| | D630Y | | | | | | |
| | RARA | NA | NA | NA | NA | NA | NA |
| | RARA | 1 | NA | NA | NA | NA | NA |
| | RARA | NA | NA | NA | NA | NA | NA |
| | RARA | 2.23E−13 | NA | NA | NA | NA | NA |
| | RARA_ | NA | 1 | 1 | 1 | NA | NA |
| | G206S | | | | | | |
| | RARA_ | 1 | 1 | 1 | 1 | NA | 1 |
| | G206S | | | | | | |
| | RARA_ | NA | 1 | 1 | 1 | 1 | NA |
| | G206S | | | | | | |
| | RARA_ | 1.59E−26 | 1 | 1 | 1 | 1 | 1 |
| | G206S | | | | | | |
| | RASGRP1 | NA | NA | NA | NA | NA | NA |
| | RASGRP1 | 1 | NA | NA | NA | NA | NA |
| | RASGRP1 | NA | NA | NA | NA | NA | NA |
| | RASGRP1 | 1 | NA | NA | NA | NA | NA |
| | RASGRP1_ | NA | 1 | 1 | 1 | NA | NA |
| | M261I | | | | | | |
| | RASGRP1_ | 1 | 1 | 1 | 1 | NA | 1 |
| | M261I | | | | | | |
| | RASGRP1_ | NA | 0.0739746 | 1 | 0.00687078 | 1.26E−36 | NA |
| | M261I | | | | | | |
| | RASGRP1_ | 1 | 1.39E−05 | 1 | 1 | 1 | 1 |
| | M261I | | | | | | |
| | RHOA | NA | NA | NA | NA | NA | NA |
| | RHOA | 1 | NA | NA | NA | NA | NA |
| | RHOA | NA | NA | NA | NA | NA | NA |
| | RHOA | 0.00018601 | NA | NA | NA | NA | NA |
| | RHOA_ | NA | 1 | 1 | 1 | NA | NA |
| | C16R | | | | | | |
| | RHOA_ | 1 | 1 | 1 | 1 | NA | 1 |
| | C16R | | | | | | |
| | RHOA_ | NA | 1 | 1 | 1 | 1 | NA |
| | C16R | | | | | | |
| | RHOA_ | 1.53E−06 | 1 | 1 | 1 | 1 | 1 |
| | C16R | | | | | | |
| | RHOA_ | NA | 1 | 1 | 1 | NA | NA |
| | G17V | | | | | | |
| | RHOA_ | 1 | 1 | 1 | 1 | NA | 1 |
| | G17V | | | | | | |
| | RHOA_ | NA | 1 | 1 | 1 | 1 | NA |
| | G17V | | | | | | |
| | RHOA_ | 0.01888866 | 1 | 1 | 1 | 1 | 1 |
| | G17V | | | | | | |
| | SELENOI- | NA | NA | NA | NA | NA | NA |
| | ABL1 | | | | | | |
| | SELENOI- | 1 | NA | NA | NA | NA | NA |
| | ABL1 | | | | | | |
| | SELENOI- | NA | NA | NA | NA | NA | NA |
| | ABL1 | | | | | | |
| | SELENOI- | 8.16E−12 | NA | NA | NA | NA | NA |
| | ABL1 | | | | | | |
| | SMARCB1 | NA | NA | NA | NA | NA | NA |
| | SMARCB1 | 1 | NA | NA | NA | NA | NA |
| | SMARCB1 | NA | NA | NA | NA | NA | NA |
| | SMARCB1 | 2.20E−08 | NA | NA | NA | NA | NA |
| | SMARCB1_ | NA | 1 | 1 | 1 | NA | NA |
| | Q368X | | | | | | |
| | SMARCB1_ | 1 | 1 | 1 | 1 | NA | 1 |
| | Q368X | | | | | | |
| | SMARCB1_ | NA | 1 | 1 | 1 | 1 | NA |
| | Q368X | | | | | | |
| | SMARCB1_ | 7.83E−09 | 1 | 1 | 1 | 1 | 1 |
| | Q368X | | | | | | |
| | SPI1- | NA | NA | NA | NA | NA | NA |
| | TCF7 | | | | | | |
| | SPI1- | 1 | NA | NA | NA | NA | NA |
| | TCF7 | | | | | | |
| | SPI1- | NA | NA | NA | NA | NA | NA |
| | TCF7 | | | | | | |
| | SPI1- | 1 | NA | NA | NA | NA | NA |
| | TCF7 | | | | | | |
| | STAT3 | NA | NA | NA | NA | NA | NA |
| | STAT3 | 1 | NA | NA | NA | NA | NA |
| | STAT3 | NA | NA | NA | NA | NA | NA |
| | STAT3 | 0.0330372 | NA | NA | NA | NA | NA |
| | STAT3_ | NA | 1 | 1 | 1 | NA | NA |
| | D661I | | | | | | |
| | STAT3_ | 1 | 1 | 1 | 1 | NA | 1 |
| | D661I | | | | | | |
| | STAT3_ | NA | 1 | 1 | 0.876204 | 1 | NA |
| | D661I | | | | | | |
| | STAT3_ | 1 | 1 | 1 | 1 | 1 | 1 |
| | D661I | | | | | | |
| | STAT3_ | NA | 1 | 1 | 1 | NA | NA |
| | G618R | | | | | | |
| | STAT3_ | 1 | 1 | 1 | 1 | NA | 1 |
| | G618R | | | | | | |
| | STAT3_ | NA | 1 | 1 | 1 | 1 | NA |
| | G618R | | | | | | |
| | STAT3_ | 1 | 1 | 1 | 1 | 1 | 1 |
| | G618R | | | | | | |
| | STAT3_ | NA | 1 | 1 | 1 | NA | NA |
| | N647I | | | | | | |
| | STAT3_ | 1 | 1 | 1 | 1 | NA | 1 |
| | N647I | | | | | | |
| | STAT3_ | NA | 1 | 1 | 1 | 1 | NA |
| | N647I | | | | | | |
| | STAT3_ | 1 | 1 | 1 | 1 | 1 | 1 |
| | N647I | | | | | | |
| | STAT5B | NA | NA | NA | NA | NA | NA |
| | STAT5B | 1 | NA | NA | NA | NA | NA |
| | STAT5B | NA | NA | NA | NA | NA | NA |
| | STAT5B | 0.00258552 | NA | NA | NA | NA | NA |
| | STAT5B_ | NA | 1 | 1 | 1 | NA | NA |
| | T628S | | | | | | |
| | STAT5B_ | 1 | 1 | 1 | 1 | NA | 1 |
| | T628S | | | | | | |
| | STAT5B_ | NA | 1 | 1 | 1 | 1 | NA |
| | T628S | | | | | | |
| | STAT5B_ | 0.00029207 | 1 | 1 | 1 | 1 | 1 |
| | T628S | | | | | | |
| | STAT5B_ | NA | 1 | 1 | 1 | NA | NA |
| | Y665F | | | | | | |
| | STAT5B_ | 1 | 1 | 1 | 1 | NA | 1 |
| | Y665F | | | | | | |
| | STAT5B_ | NA | 1 | 1 | 1 | 1 | NA |
| | Y665F | | | | | | |
| | STAT5B_ | 0.00115151 | 1 | 1 | 1 | 1 | 1 |
| | Y665F | | | | | | |
| | TNFRSF1B | NA | NA | NA | NA | NA | NA |
| | TNFRSF1B | 1 | NA | NA | NA | NA | NA |
| | TNFRSF1B | NA | NA | NA | NA | NA | NA |
| | TNFRSF1B | 0.00029207 | NA | NA | NA | NA | NA |
| | TNFRSF1B_ | NA | 1 | 1 | 1 | NA | NA |
| | G256C | | | | | | |
| | TNFRSF1B_ | 1 | 1 | 0.090972 | 1 | NA | 1 |
| | G256C | | | | | | |
| | TNFRSF1B_ | NA | 1 | 1 | 1 | 1 | NA |
| | G256C | | | | | | |
| | TNFRSF1B_ | 0.00450072 | 1 | 1 | 1 | 1 | 1 |
| | G256C | | | | | | |
| | TNFRSF1B_ | NA | 1 | 9.96E−06 | 1 | NA | NA |
| | T377I | | | | | | |
| | TNFRSF1B_ | 1 | 1 | 5.03E−10 | 1 | NA | 1 |
| | T377I | | | | | | |
| | TNFRSF1B_ | NA | 0.0108927 | 1.54E−05 | 1 | 1 | NA |
| | T377I | | | | | | |
| | TNFRSF1B_ | 1.53E−06 | 1 | 0.0056259 | 1 | 1 | 1 |
| | T377I | | | | | | |
| | TP53 | NA | NA | NA | NA | NA | NA |
| | TP53 | 1 | NA | NA | NA | NA | NA |
| | TP53 | NA | NA | NA | NA | NA | NA |
| | TP53 | 1 | NA | NA | NA | NA | NA |
| | TP53_ | NA | 1 | 1 | 1 | NA | NA |
| | R273P | | | | | | |
| | TP53_ | 1 | 1 | 1 | 1 | NA | 1 |
| | R273P | | | | | | |
| | TP53_ | NA | 1 | 6.15E−07 | 1 | 1 | NA |
| | R273P | | | | | | |
| | TP53_ | 0.0292068 | 1 | 1 | 1 | 1 | 1 |
| | R273P | | | | | | |
| | VAV1 | NA | NA | NA | NA | NA | NA |
| | VAV1 | 1 | NA | NA | NA | NA | NA |
| | VAV1 | NA | NA | NA | NA | NA | NA |
| | VAV1 | 1 | NA | NA | NA | NA | NA |
| | VAV1_ | NA | 1 | 1 | 1 | NA | NA |
| | D797H | | | | | | |
| | VAV1_ | 1 | 1 | 1 | 1 | NA | 1 |
| | D797H | | | | | | |
| | VAV1_ | NA | 1 | 1 | 1 | 1 | NA |
| | D797H | | | | | | |
| | VAV1_ | 1 | 1 | 1 | 1 | 1 | 1 |
| | D797H | | | | | | |
| | VAV1_ | NA | 1 | 1 | 1 | NA | NA |
| | R798Q | | | | | | |
| | VAV1_ | 1 | 1 | 1 | 1 | NA | 1 |
| | R798Q | | | | | | |
| | VAV1_ | NA | 1 | 1 | 1 | 1 | NA |
| | R798Q | | | | | | |
| | VAV1_ | 1 | 1 | 1 | 1 | 1 | 1 |
| | R798Q | | | | | | |
| | VAV1- | NA | NA | NA | NA | NA | NA |
| | GSS | | | | | | |
| | VAV1- | 1 | NA | NA | NA | NA | NA |
| | GSS | | | | | | |
| | VAV1- | NA | NA | NA | NA | NA | NA |
| | GSS | | | | | | |
| | VAV1- | 1 | NA | NA | NA | NA | NA |
| | GSS | | | | | | |

### REFERENCES

1 H., O'Connor, R. S., Kawalekar, O. U., Gliassemi, S. & Milone, M. C. CAR T cell immunotherapy for human cancer. Science 359, 1361-1365, doi: 10. 1126/science.aar6711 (2018).
2 Hou, A. J., Chen, L. C. & Chen, Y. Y. Navigating CAR-T cells through the solid-tumour microenvironment. Nature Reviews Drug Discovery 20, 531-550, doi:10.1038/s41573-021-00189-2 (2021).
3 Larson, R. C. & Maus, M. V. Recent advances and discoveries in the mechanisms and functions of CAR T cells. Nature Reviews Cancer 21, 145-161, doi:10.1038/s41568-020-00323-z (2021).
4 Long, A. H. et al. 4-1BB costimulation ameliorates T cell exhaustion induced by tonic signaling of chimeric antigen receptors. Nat Med 21, 581-590, doi:10.1038/nm.3838 (2015).
Maude, S. L. et al. Chimeric Antigen Receptor T Cells for Sustained Remissions in Leukemia. New England Journal of Medicine 371, 1507-1517, doi:10.1056/NEJMoa1407222 (2014).
6 Kloss, C. C. et al. Dominant-Negative TGF-β Receptor Enhances PSMA-Targeted Human CAR T Cell Proliferation And Augments Prostate Cancer Eradication. Mol Ther 26, 1855-1866, doi:10.1016/j.ymthe.2018.05.003 (2018).
7 Wei, J. et al. Targeting REGNASE-1 programs long-lived effector T cells for cancer therapy. Nature 576, 471-476, doi:10.1038/s41586-019-1821-z (2019).
8 Legut, M. et al. A genome-scale screen for synthetic drivers of T cell proliferation. Nature 603, 728-735, doi:10.1038/s41586-022-04494-7 (2022).
9 Shifrut, E. et al. Genome-wide CRISPR Screens in Primary Human T Cells Reveal Key Regulators of Immune Function. Cell 175, 1958-1971.e1915, doi:https://doi.org/10.1016/j.cell.2018.10.024 (2018).
10 Schmidt, R. et al. CRISPR activation and interference screens decode stimulation responses in primary human T cells. Science 375, eabj4008, doi:10.1126/science.abj4008.
11 Sutra Del Galy, A. et al. In vivo genome-wide CRISPR screens identify SOCS1 as intrinsic checkpoint of CD4(+) T(H)1 cell response. Sci Immunol 6, eabe8219, doi:10.1126/sciimmunol.abe8219 (2021).
12 Dong, M. B. et al. Systematic Immunotherapy Target Discovery Using Genome-Scale In Vivo CRISPR Screens in CD8 T Cells. Cell 178, 1189-1204.e1123, doi:10.1016/j.cell.2019.07.044 (2019).
13 Zhou, P. et al. In vivo discovery of immunotherapy targets in the tumour microenvironment. Nature 506, 52-57, doi:10.1038/nature12988 (2014).
14 Roth, T. L. et al. Pooled Knockin Targeting for Genome Engineering of Cellular Immunotherapies. Cell 181, 728-744.e721, doi:10.1016/j.cell.2020.03.039 (2020).
15 Mustjoki, S. & Young, N. S. Somatic Mutations in “Benign” Disease. N Engl J Med 384, 2039-2052, doi:10.1056/NEJMra2101920 (2021).
16 Kadin, M. E. et al. Loss of receptors for transforming growth factor beta in human T-cell malignancies. Proc Natl Acad Sci USA 91, 6002-6006, doi:10.1073/pnas.91.13.6002 (1994).
17 Park, J. et al. Integrated genomic analyses of cutaneous T-cell lymphomas reveal the molecular bases for disease heterogeneity. Blood 138, 1225-1236, doi:10.1182/blood.2020009655 (2021).
18 Wartewig, T. et al. PD-1 is a haploinsufficient suppressor of T cell lymphomagenesis. Nature 552, 121-125, doi:10.1038/nature24649 (2017).
19 Kataoka, K. et al. Integrated molecular analysis of adult T cell leukemia/lymphoma. Nat Genet 47, 1304-1315, doi:10.1038/ng.3415 (2015).
20 Kleppe, M. et al. Mutation analysis of the tyrosine phosphatase PTPN2 in Hodgkin's lymphoma and T-cell non-Hodgkin's lymphoma. Haematologica 96, 1723-1727, doi:10.3324/haematol.2011.041921 (2011).
21 Stadtmauer Edward, A. et al. CRISPR-engineered T cells in patients with refractory cancer. Science 367, eaba7365, doi:10.1126/science.aba7365 (2020).
22 Fraietta, J. A. et al. Disruption of TET2 promotes the therapeutic efficacy of CD19-targeted T cells. Nature 558, 307-312, doi:10.1038/s41586-018-0178-z (2018).
23 Kumar, J. et al. Deletion of Cbl-b inhibits CD8&lt;sup&gt;+&lt;/sup&gt; T-cell exhaustion and promotes CAR T-cell function. Journal for ImmunoTherapy of Cancer 9, e001688, doi:10.1136/jitc-2020-001688 (2021).
24 Prinzing, B. et al. Deleting DNMT3A in CAR T cells prevents exhaustion and enhances antitumor activity. Science Translational Medicine 13, eabh0272, doi:10.1126/scitranslmed.abh0272.
25 Wiede, F. et al. PTPN2 phosphatase deletion in T cells promotes anti-tumour immunity and CAR T-cell efficacy in solid tumours. The EMBO Journal 39, e103637, doi:https://doi.org/10.15252/embj.2019103637 (2020).
26 Park, J. et al. Genomic analysis of 220 CTCLs identifies a novel recurrent gain-of-function alteration in RLTPR (p.Q575E). Blood 130, 1430-1440, doi:10.1182/blood-2017-02-768234 (2017).
27 Daniels, J. et al. Cellular origins and genetic landscape of cutaneous gamma delta T cell lymphomas. Nature communications 11, 1806-1806, doi:10.1038/s41467-020-15572-7 (2020).
28 Jutz, S. et al. Assessment of costimulation and coinhibition in a triple parameter T cell reporter line: Simultaneous measurement of NF-κB, NFAT and AP-1. J Immunol Methods 430, 10-20, doi:10.1016/j.jim.2016.01.007 (2016).
29 Patel, V. M. et al. Frequent and Persistent PLCG1 Mutations in Sézary Cells Directly Enhance PLC71 Activity and Stimulate NFκB, AP-1, and NFAT Signaling. J Invest Dermatol 140, 380-389.e384, doi:10.1016/j.jid.2019.07.693 (2020).
30 Ungewickell, A. et al. Genomic analysis of mycosis fungoides and Sézary syndrome identifies recurrent alterations in TNFR2. Nature genetics 47, 1056-1060, doi:10.1038/ng.3370 (2015).
31 Robles-Valero, J. et al. Cancer-associated mutations in VAV1 trigger variegated signaling outputs and T-cell lymphomagenesis. Embo j 40, e108125, doi:10.15252/embj.2021108125 (2021).
32 Vallois, D. et al. Activating mutations in genes related to TCR signaling in angioimmunoblastic and other follicular helper T-cell-derived lymphomas. Blood 128, 1490-1502, doi:10.1182/blood-2016-02-698977 (2016).
33 Chen, S. S., Hu, Z. & Zhong, X.-P. Diacylglycerol Kinases in T Cell Tolerance and Effector Function. Frontiers in Cell and Developmental Biology 4, doi:10.3389/fcell.2016.00130 (2016).
34 Martinez, G. J. et al. The transcription factor NFAT promotes exhaustion of activated CD8+ T cells. Immunity 42, 265-278, doi:10.1016/j.immuni.2015.01.006 (2015).
35 Rafiq, S., Hackett, C. S. & Brentjens, R. J. Engineering strategies to overcome the current roadblocks in CAR T cell therapy. Nature Reviews Clinical Oncology 17, 147-167, doi:10.1038/s41571-019-0297-y (2020).
36 Sharma, P. & Allison, J. P. Immune checkpoint targeting in cancer therapy: toward combination strategies with curative potential. Cell 161, 205-214, doi:10.1016/j.cell.2015.03.030 (2015).
37 Schmitz, R. et al. Burkitt lymphoma pathogenesis and therapeutic targets from structural and functional genomics. Nature 490, 116-120, doi:10.1038/nature11378 (2012).
38 Liu, Y. et al. The genomic landscape of pediatric and young adult T-lineage acute lymphoblastic leukemia. Nature genetics 49, 1211-1218, doi:10.1038/ng.3909 (2017).
39 Lynn, R. C. et al. c-Jun overexpression in CAR T cells induces exhaustion resistance. Nature 576, 293-300, doi:10.1038/s41586-019-1805-z (2019).
40 Kahan, S. M. et al. Intrinsic IL-2 production by effector CD8 T cells affects IL-2 signaling and promotes fate decisions, stemness, and protection. Sci Immunol 7, eabl6322, doi:10.1126/sciimmunol.abl6322 (2022).
41 Wang, L. et al. Genomic profiling of Sézary syndrome identifies alterations of key T cell signaling and differentiation genes. Nature genetics 47, 1426-1434, doi:10.1038/ng.3444 (2015).
42 Ruland, J. & Hartjes, L. CARD-BCL-10-MALT1 signalling in protective and pathological immunity. Nat Rev Immunol 19, 118-134, doi:10.1038/s41577-018-0087-2 (2019).
43 Jattani, R. P., Tritapoe, J. M. & Pomerantz, J. L. Intramolecular Interactions and Regulation of Cofactor Binding by the Four Repressive Elements in the Caspase Recruitment Domain-containing Protein 11 (CARD11) Inhibitory Domain. J Biol Chem 291, 8338-8348, doi:10.1074/jbc.M116.717322 (2016).
44 Burke, J. E. Structural Basis for Regulation of Phosphoinositide Kinases and Their Involvement in Human Disease. Molecular Cell 71, 653-673, doi:https://doi.org/10.1016/j.molcel.2018.08.005 (2018).
45 Kutzner, K. et al. Phosphorylation of serine-893 in CARD11 suppresses the formation and activity of the CARD11-BCL10-MALT1 complex in T and B cells. Science Signaling 15, eabk3083, doi:10.1126/scisignal.abk3083.
46 Li, S., Yang, X., Shao, J. & Shen, Y. Structural Insights into the Assembly of CARMA1 and BCL10. PLOS ONE 7, e42775, doi:10.1371/journal.pone.0042775 (2012).
47 Mingueneau, M. et al. Single-cell mass cytometry of TCR signaling: Amplification of small initial differences results in low ERK activation in NOD mice. Proceedings of the National Academy of Sciences 111, 16466-16471, doi:10.1073/pnas.1419337111 (2014).
48 Yamamoto, Y. & Gaynor, R. B. IkappaB kinases: key regulators of the NF-kappaB pathway. Trends Biochem Sci 29, 72-79, doi:10.1016/j.tibs.2003.12.003 (2004).
49 Fan, X., Quezada, S. A., Sepulveda, M. A., Sharma, P. & Allison, J. P. Engagement of the ICOS pathway markedly enhances efficacy of CTLA-4 blockade in cancer immunotherapy. Journal of Experimental Medicine 211, 715-725, doi:10.1084/jem.20130590 (2014).
50 Massarelli, E. et al. High OX-40 expression in the tumor immune infiltrate is a favorable prognostic factor of overall survival in non-small cell lung cancer. J Immunother Cancer 7, 351, doi:10.1186/s40425-019-0827-2 (2019).
51 Bardet, M. et al. The T-cell fingerprint of MALT1 paracaspase revealed by selective inhibition. Immunol Cell Biol 96, 81-99, doi:10.1111/imcb.1018 (2018).
52 Jiang, T., Zhou, C. & Ren, S. Role of IL-2 in cancer immunotherapy. Oncoimmunology 5, e1163462, doi:10.1080/2162402x.2016.1163462 (2016).
53 Thommen, D. S. & Schumacher, T. N. T Cell Dysfunction in Cancer. Cancer Cell 33, 547-562, doi:10.1016/j.ccell.2018.03.012 (2018).
54 Guedan, S. et al. Enhancing CAR T cell persistence through ICOS and 4-1BB costimulation. JCI Insight 3, doi:10.1172/jci.insight.96976 (2018).
55 Li, Q.-X., Feuer, G., Ouyang, X. & An, X. Experimental animal modeling for immuno-oncology. Pharmacology & Therapeutics 173, 34-46, doi:https://doi.org/10.1016/j.pharmthera.2017.02.002 (2017).
56 Kalbasi, A. et al. Potentiating adoptive cell therapy using synthetic IL-9 receptors. Nature 607, 360-365, doi:10.1038/s41586-022-04801-2 (2022).
57 Zhang, Y., Liu, Z., Wei, W. & Li, Y. TCR engineered T cells for solid tumor immunotherapy. Experimental Hematology & Oncology 11, 38, doi:10.1186/s40164-022-00291-0 (2022).
58 Overwijk, W. W. & Restifo, N. P. B16 as a mouse model for human melanoma. Curr Protoc Immunol Chapter 20, Unit-20.21, doi:10.1002/0471142735.im2001s39 (2001).
59 Eyquem, J. et al. Targeting a CAR to the TRAC locus with CRISPR/Cas9 enhances tumour rejection. Nature 543, 113-117, doi:10.1038/nature21405 (2017).
60 McLellan, A. D. & Ali Hosseini Rad, S. M. Chimeric antigen receptor T cell persistence and memory cell formation. Immunol Cell Biol 97, 664-674, doi:10.1111/imcb.12254 (2019).
61 Klichinsky, M. et al. Human chimeric antigen receptor macrophages for cancer immunotherapy. Nat Biotechnol 38, 947-953, doi:10.1038/s41587-020-0462-y (2020).
62 Rezvani, K. Adoptive cell therapy using engineered natural killer cells. Bone Marrow Transplantation 54, 785-788, doi:10.1038/s41409-019-0601-6 (2019).
63 Kabelitz, D., Serrano, R., Kouakanou, L., Peters, C. & Kalyan, S. Cancer immunotherapy with 76 T cells: many paths ahead of us. Cellular & Molecular Immunology 17, 925-939, doi:10.1038/s41423-020-0504-x (2020).
64 Wennhold, K., Shimabukuro-Vornhagen, A. & von Bergwelt-Baildon, M. B Cell-Based Cancer Immunotherapy. Transfusion Medicine and Hemotherapy 46, 36-46, doi:10.1159/000496166 (2019).
65 Zhu, I. et al. Modular design of synthetic receptors for programmed gene regulation in cell therapies. Cell 185, 1431-1443.e1416, doi:10.1016/j.cell.2022.03.023 (2022).
66 Zhao, H. et al. Genome-wide fitness gene identification reveals Roquin as a potent suppressor of CD8 T cell expansion and anti-tumor immunity. Cell Rep 37, 110083, doi:10.1016/j.celrep.2021.110083 (2021).
67 Chen L, Flies D B. Molecular mechanisms of T cell co-stimulation and co-inhibition. Nat Rev Immunol 2013 134. 2013; 13(4):227-242.
68 McCubrey J A, Steelman L S, Bäsecke J, Martelli A M. Raf/mek/erk signaling. Target Ther Acute Myeloid Leuk. January 2015:275-305.
69 Jutz, S. et al. Assessment of costimulation and coinhibition in a triple parameter T cell reporter line: Simultaneous measurement of NF-κB, NFAT and AP-1. J Immunol Methods 430, 10-20, doi:10.1016/j.jim.2016.01.007 (2016).
70 Love M I, Huber W, Anders S. Moderated estimation of fold change and dispersion for RNA-seq data with DESeq2. Genome Biol. 2014; 15(12):1-21.
71 J. Ruland, L. Hartjes, CARD-BCL-10-MALT1 signalling in protective and pathological immunity. Nat Rev Immunol 19, 118-134 (2019). ADDIN EN.REFLIST
72 Müller MR, Rao A. NFAT, immunity and cancer: a transcription factor comes of age. Nat Rev Immunol 2010 109. 2010; 10(9):645-656
73 Jang H J, Suh P G, Lee Y J, Shin K J, Cocco L, Chae Y C. PLC71: Potential arbitrator of cancer progression. Adv Biol Regul. 2018; 67: 179-189.
74 Patterson R L, Van Rossum D B, Nikolaidis N, Gill D L, Snyder S H. Phospholipase C-y: diverse roles in receptor-mediated calcium signaling. Trends Biochem Sci. 2005; 30(12):688-697.
75 Patel V M et al., Frequent and Persistent PLCG1 Mutations in Sézary Cells Directly Enhance PLCT1 Activity and Stimulate NFκB, AP-1, and NFAT Signaling. J Invest Dermatol 140, 380-389.e384 (2020).
76 Park, J. et al. Integrated genomic analyses of cutaneous T-cell lymphomas reveal the molecular bases for disease heterogeneity. Blood 138, 1225-1236, doi:10.1182/blood.2020009655 (2021).
77 Porter, D. L., Levine, B. L., Kalos, M., Bagg, A. & June, C. H. Chimeric Antigen Receptor-Modified T Cells in Chronic Lymphoid Leukemia. New England Journal of Medicine 365, 725-733, doi:10.1056/NEJMoa1103849 (2011).
78 Shaulian E, Karin M. AP-1 as a regulator of cell life and death. Nat Cell Biol 2002 45. 2002; 4(5):E131-E136.
79 Taniguchi K, Karin M. NF-κB, inflammation, immunity and cancer: coming of age. Nat Rev Immunol 2018 185. 2018; 18(5):309-324. doi:10.1038/nri.2017.142.
80 Villarino A V., Kanno Y, O'Shea J J. Mechanisms and consequences of Jak-STAT signaling in the immune system. Nat Immunol 2017184. 2017; 18(4):374-384.
81 Wei, J. et al. Targeting REGNASE-1 programs long-lived effector T cells for cancer therapy. Nature 576, 471-476, doi:10.1038/s41586-019-1821-z (2019).
82 L. Wang et al., Genomic profiling of Sézary syndrome identifies alterations of key T cell signaling and differentiation genes. Nature genetics 47, 1426-1434 (2015).

What is claimed:

1. A method of treating a subject with a disease, the method comprising administering to the subject an engineered cell comprising a polypeptide or a nucleic acid encoding the polypeptide, the polypeptide comprising:
a. a caspase-associated recruitment domain (CARD) or a functional fragment thereof; and
b. a Src Homology region 2 (SH2) domain.

2. The method of claim 1, wherein the polypeptide comprises a coiled-coil domain.

3. The method of claim 2, wherein both the CARD and the coiled-coil domain are from CARD11.

4. The method of claim 1, wherein the SH2 domain is from PIK3R3.

5. The method of claim 1, wherein the polypeptide is not a transmembrane receptor.

6. The method of claim 1, wherein the disease is a cancer.

7. The method of claim 6, wherein the cancer is a solid tumor cancer or a hematological cancer.

8. The method of claim 7, wherein the solid tumor cancer is small cell lung cancer, colorectal cancer, testicular cancer, ovarian cancer, melanoma, prostate cancer, breast cancer, non-small cell lung cancer, gastric cancer, esophageal cancer, liver cancer, kidney cancer, head & neck cancer, glioblastoma, neuroblastoma, soft tissue sarcoma, uterine cancer, brain cancer, skin cancer, renal cancer, bladder cancer, pancreatic cancer, thyroid cancer, eye cancer, gastrointestinal cancer, carcinoma, or sarcoma.

9. The method of claim 6, wherein the cancer expresses CD1, CD1a, CD1b, CD1c, CD1d, CD1e, CD2, CD3d, CD3e, CD3g, CD3F, CD4, CD5, CD7, CD8a, CD8b, CD19, CD20, CD21, CD22, CD23, CD24, CD25, CD27, CD28, CD30, CD33, CD34, CD38, CD40, CD44v6, CD45, CD48, CD52, CD59, CD66, CD70, CD71, CD72, CD73, CD79A, CD79B, CD80 (B7.1), CD86 (B7.2), CD94, CD95, CD97, CD123, CD134, CD140 (PDGFR4), CD152, CD154, CD158, CD171, CD178, CD179, CD179a, CD181 (CXCR1), CD182 (CXCR2), CD183 (CXCR3), CD210, CD246, CD252, CD253, CD261, CD262, CD273 (PD-L2), CD274 (PD-L1), CD276 (B7H3), CD279, CD295, CD339 (JAG1), CD340 (HER2), CEA, CLL-1, CS1, DLL3, LY6G6D, Claudin 6, GCC, p53R175H, PRAME, EGFR, FGFR2, AFP, CA125, MUC-1, MAGE, alkaline phosphatase, placental-like 2 (ALPPL2), B-cell maturation antigen (BCMA), green fluorescent protein (GFP), enhanced green fluorescent protein (eGFP), Claudin18.2, PSMA, ROR1, Mesothelin, IL13Ra2, FAP, signal regulatory protein α (SIRPα), TCRalpha, TCRbeta, TSHR, EGFRvIII, GD2, GD3, Tn Ag, ROR1, ROR2, GPC1, GPC2, FLT3, FAP, TAG72, CEA, EPCAM, KIT, IL-13Ra2, IL-11Ra, PSCA, PRSS21, VEGFR2, LewisY, PDGFR-beta, SSEA-4, folate receptor alpha, ERBB2 (Her2/neu), MUC16, NCAM, prostase, PAP, ELF2M, Ephrin B2, IGF-I receptor, CAIX, LMP2, gp100, bcr-abl, tyrosinase, EphA2, fucosyl GM1, sLe, GM3, TGS5, HMWMAA, o-acetyl-GD2, folate receptor beta, TEM1/CD248, TEM7R, GPRC5D, CXORF61, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-la, MAGE-Al, legumain, HPV E6,E7, ETV6-AML, sperm protein 17, XAGE1, Tie 2, MAD-CT-1, MAD-CT-2, Fos-related antigen 1, p53, p53 mutant, KRAS, mutant KRAS, KRAS G12D, prostein, surviving, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2,LAIR1, FCAR, LILRA2, CD300LF, CLEC12A, BST2, EMR2, LY75, GPC3, FCRL5, IGLL1, PSMA, an extracellular portion of APRIL protein, or any combinations thereof.

10. The method of claim 6, wherein the cancer expresses BCMA, CD19, CD20, CD22, CD70, CD79A, CD79B, CD276 (B7H3), Claudin 6, Claudin 18.2, DLL3, GCC, GD2, GD3, GPC3, GPRC5D, LY6G6D, p53R175H, PRAME, ROR1, or any combinations thereof.

11. The method of claim 1, wherein the disease is an autoimmune disease.

12. The method of claim 1, wherein the method of treatment does not comprise administration of a lymphodepletive agent within 7 days prior to administration of the engineered cell.

13. The method of claim 1, wherein the method of treatment does not comprise administration of cyclophosphamide, fludarabine, and/or bendamustine within 7 days prior to administration of the engineered cell.

14. The method of claim 1, wherein the method of treatment does not comprise administration of at least 600,000 IU/kg of IL-2 every 8 hours.

15. The method of claim 1, wherein the method of treatment does not comprise administration of a checkpoint therapy.

16. The method of claim 1, wherein the engineered cell has reduced exhaustion, increased proliferative capacity, enhanced replicative lifespan, decreased replicative senescence, enhanced anti-tumor effect, reduced dysfunction, enhanced persistence, or increased intratumoral presence in vivo, compared to a control cell that does not comprise the polypeptide or the nucleic acid.

17. The method of claim 1, wherein the engineered cell has increased or decreased signaling through CARD11-BCL10-MALT1 complex, NF-κB, AP-1, NFAT, JAK/STAT, or MEK/ERK pathway, compared to a control cell that does not comprise the polypeptide or the nucleic acid.

18. The method of claim 1, wherein the CARD comprises or consists of a sequence having at least 85% identity to any one of SEQ ID NO: 261-289.

19. The method of claim 1, wherein the polypeptide comprises or consists of a sequence having at least 85% identity to any one of SEQ ID NO: 206, 226, 228, 230, 232, 234, 236, 238, 240, 242, 244, 246, 248, 250, 252, 254, or 256.

20. The method of claim 1, wherein the nucleic acid encoding the polypeptide comprises or consists of a sequence having at least 85% identity to any one of SEQ ID NO: 205, 225, 227, 229, 231, 233, 235, 237, 239, 241, 243, 245, 247, 249, 251, 253, or 255.

21. The method of claim 2, wherein the coiled-coil domain comprises or consists of a sequence having at least 85% identity to any one of SEQ ID NOs: 290-293.

22. The method of claim 2, wherein the coiled-coil domain comprises or consists of a sequence having at least 85% identity to SEQ ID NO: 290.

23. The method of claim 1, wherein the SH2 domain comprises or consists of a sequence having at least 85% identity to any one of SEQ ID NOs: 305 and 307-437.

24. The method of claim 1, wherein the SH2 domain comprises or consists of a sequence having at least 85% identity to SEQ ID NO: 305.

25. The method of claim 1, wherein the engineered cell further comprises a chimeric antigen receptor (CAR), a T cell receptor (TCR), a cytokine, a chemokine, a growth factor, a safety switch, or any combinations thereof.

26. The method of claim 25, wherein the CAR or the TCR have specificity for a target antigen selected from the group consisting of CD1, CD1a, CD1b, CD1c, CD1d, CD1e, CD2, CD3d, CD3e, CD3g, CD3F, CD4, CD5, CD7, CD8a, CD8b, CD19, CD20, CD21, CD22, CD23, CD24, CD25, CD27, CD28, CD30, CD33, CD34, CD38, CD40, CD44v6, CD45, CD48, CD52, CD59, CD66, CD70, CD71, CD72, CD73, CD79A, CD79B, CD80 (B7.1), CD86 (B7.2), CD94, CD95, CD97, CD123, CD134, CD140 (PDGFR4), CD152, CD154, CD158, CD171, CD178, CD179, CD179a, CD181 (CXCR1), CD182 (CXCR2), CD183 (CXCR3), CD210, CD246, CD252, CD253, CD261, CD262, CD273 (PD-L2), CD274 (PD-L1), CD276 (B7H3), CD279, CD295, CD339 (JAG1), CD340 (HER2), CEA, CLL-1, CS1, DLL3, LY6G6D, Claudin 6, GCC, p53R175H, PRAME, EGFR, FGFR2, AFP, CA125, MUC-1, MAGE, alkaline phosphatase, placental-like 2 (ALPPL2), B-cell maturation antigen (BCMA), green fluorescent protein (GFP), enhanced green fluorescent protein (eGFP), Claudin 18.2, PSMA, ROR1, Mesothelin, IL13Ra2, FAP, signal regulatory protein α (SIRPα), TCRalpha, TCRbeta, TSHR, EGFRvIII, GD2, GD3, Tn Ag, ROR1, ROR2, GPC1, GPC2, FLT3, FAP, TAG72, CEA, EPCAM, KIT, IL-13Ra2, IL-11Ra, PSCA, PRSS21, VEGFR2, LewisY, PDGFR-beta, SSEA-4, folate receptor alpha, ERBB2 (Her2/neu), MUC16, NCAM, prostase, PAP, ELF2M, Ephrin B2, IGF-I receptor, CAIX, LMP2, gp100, bcr-abl, tyrosinase, EphA2, fucosyl GM1, sLe, GM3, TGS5, HMWMAA, o-acetyl-GD2, folate receptor beta, TEM1/CD248, TEM7R, GPRC5D, CXORF61, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-la, MAGE-Al, legumain, HPV E6,E7, ETV6-AML, sperm protein 17, XAGE1, Tie 2, MAD-CT-1, MAD-CT-2, Fos-related antigen 1, p53, p53 mutant, KRAS, mutant KRAS, KRAS G12D, prostein, surviving, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, LAIR1, FCAR, LILRA2, CD300LF, CLEC12A, BST2, EMR2, LY75, GPC3, FCRL5, IGLL1, PSMA, an extracellular portion of APRIL protein, and any combinations thereof.

27. The method of claim 25, wherein the CAR or the TCR have specificity for a target antigen selected from the group consisting of BCMA, CD19, CD20, CD22, CD70, CD79A, CD79B, CD276 (B7H3), Claudin 6, Claudin 18.2, DLL3, GCC, GD2, GD3, GPC3, GPRC5D, LY6G6D, p53R175H, PRAME, ROR1, and any combinations thereof.

28. The method of claim 1, wherein the engineered cell is selected from the group consisting of an immune cell, a T cell, a CD4+ T cell, a CD8+ T cell, a regulatory T cell (Treg), a gamma delta T cell (76T), an invariant natural killer T (iNKT) cell, a mucosal associated invariant T (MAIT) cell, a macrophage, a monocyte, a natural killer (NK) cell, a tumor infiltrating lymphocyte (TIL), a cytotoxic T cell, a T helper cell, a memory T cell, a central memory T (TCM) cell, a stem memory T (TSCM) cell, a stem-cell-like memory T cell, a stem-like memory T cell, an effector memory T (TEM) cell, a TEMRA (CD45RA+) cell, an effector T cell, a Th1 cell, a Th2 cell, a Th9 cell, a Th17 cell, a Th22 cell, a Tfh (follicular helper) cell, a natural killer T (NKT) cell, a transitional memory T (TTM) cell, a terminal effector T (TTE) cell, a naïve T (TN) cell, a hematopoietic stem cell, and a progenitor cell of lymphoid lineage.

29. The method of claim 1, wherein the engineered cell is selected from the group consisting of a T cell, a macrophage, a monocyte, and a natural killer (NK) cell.

30. The method of claim 1, wherein expression of the polypeptide is controlled by a promoter.

31. The method of claim 30, wherein the promoter is selected from the group consisting of a CD4 promoter, a CD8a promoter, a CD8b promoter, a TCRa promoter, a TCRb promoter, a CD3d promoter, a CD3g promoter, a CD3e promoter, a CD3z promoter, a minimal TATA promoter, a pGK, actin promoter, a CD25 promoter, an IL2 promoter, an IL7 promoter, an IL15 promoter, a KLRG-1 promoter, a HLA-DR promoter, a CD38 promoter, a CD69 promoter, a Ki-67 promoter, a CD11a promoter, a CD58 promoter, a CD99 promoter, a CD62L promoter, a CD103 promoter, a CCR4 promoter, a CCR5 promoter, a CCR6 promoter, a CCR9 promoter, a CCR10 promoter, a CXCR3 promoter, a CXCR4 promoter, a CLA promoter, a Granzyme A promoter, a Granzyme B promoter, a Perforin promoter, a CD57 promoter, a CD161 promoter, an IL-18Ra promoter, a CD69 promoter, a GzmB promoter, a T-bet promoter, an IFNgamma promoter, an IL4 promoter, a GATA3 promoter, an IL1 promoter, an IL5 promoter, an IL6 promoter, an IL13 promoter, an IL10 promoter, an IL17A promoter, an IL6 promoter, an IL21 promoter, an IL23R promoter, a FoxP3 promoter, a CTLA4 promoter, a CD25 promoter, a CD45RO promoter, a CCR7 promoter, a CD28 promoter, a CD95 promoter, a CD28 promoter, a CD27 promoter, a CD127 promoter, a CD122 promoter, a CD132 promoter, a c-Kit promoter, a nuclear factor of activated T cells (NFAT) promoter, a programmed death 1 (PD-1) promoter, a T cell immunoglobulin mucin-3 (TIM-3) promoter, a cytotoxic T lymphocyte antigen-4 (CTLA4) promoter, a lymphocyte-activation protein 3 (LAG-3) promoter, a tumor necrosis factor (TNF)-related apoptosis-inducing ligand (TRAIL) promoter, a B- and T-lymphocyte attenuator (BTLA) promoter, a CD25 promoter, a CD69 promoter, a Fas ligand (FasL) promoter, a TIGIT promoter, a TGF-beta promoter, a T-bet promoter, an Eomes promoter, a CD45RA promoter, a 2B4 promoter, a Type I interferon (IFN) alpha, a Type I IFN beta promoter, an IFN gamma promoter, an IRF3 promoter, an IRF7 promoter, a NFkB promoter, an AP-1 promoter, a TNF-alpha promoter, a CD130 promoter, a NR4A1 promoter, a NR4A2, a NR4A3 promoter, and any combination thereof.

32. The method of claim 1, wherein the nucleic acid is inserted in a TCR alpha locus of the engineered cell.

33. The method of claim 1, wherein the engineered cell has reduced or eliminated expression of an endogenous T cell receptor (TCR).

34. The method of claim 1, wherein the engineered cell is an autologous cell.

35. The method of claim 1, wherein the engineered cell is an allogeneic cell.

36. A method of enhancing in vivo persistence of a T cell in a subject in need thereof, comprising introducing to the T cell a nucleic acid encoding a polypeptide comprising:
a. a caspase-associated recruitment domain (CARD) or a functional fragment thereof; and
b. a Src Homology region 2 (SH2) domain.

37. The method of claim 36, wherein the in vivo persistence of the T cell is measured by:
(i) the number of accumulated T cells in a tumor;
(ii) intra-tumoral expression of TNF-α, IFNγ, IL-2, or any combination thereof; or
(iii) expression of stemness-associated transcription factor TCF1 in tumor infiltrating lymphocytes.


SEQUENCE LISTING (437 sequences)

SEQ ID NO: 1 - DNA, 2118 bp, Synthetic construct
  source 1..2118: mol_type = other DNA
atggcctcgccggctgacagctgtatccagttcacccgccatgccagtgatgttcttctcaaccttaatcgtctccggagtcgagacatcttgactgatgttgtcattgttgtgagccgtgagcagtttagagcccataaaacggtcctcatggcctgcagtggcctgttctatagcatctttacagaccagttgaaatgcaaccttagtgtgatcaatctagatcctgagatcaaccctgagggattctgcatcctcctggacttcatgtacacatctcggctcaatttgcgggagggcaacatcatggctgtgatggccacggctatgtacctgcagatggagcatgttgtggacacttgccggaagtttattaaggccagtgaagcagagatggtttctgccatcaagcctcctcgtgaagagttcctcaacagccggatgctgatgccccaagacatcatggcctatcggggttgtgaggtggtggagaacaacctgccactgaggagcgcccctgggtgtgagagcagagcctttgcccccagcctgtacagtggcctgtccacaccgccagcctcttattccatgtacagccacctccctgtcagcagcctcctcttctccgatgaggagtttcgggatgtccggatgcctgtggccaaccccttccccaaggagcgggcactcccatgtgatagtgccaggccagtccctggtgagtacagccggccgactttggaggtgtcccccaatgtgtgccacagcaatatctattcacccaaggaaacaatcccagaagaggcacgaagtgatatgcactacagtgtggctgagggcctcaaacctgctgccccctcagcccgaaatgccccctacttcccttgtgacaaggccagcaaagaagaagagagaccctcctcggaagatgagattgccctgcatttcgagccccccaatgcacccctgaaccggaagggtctggttagtccacagagcccccagaaatctgactgccagcccaactcgcccacagagtcctgcagcagtaagaatgcctgcatcctccaggcttctggctcccctccagccaagagccccactgaccccaaagcctgcaactggaagaaatacaagttcatcgtgctcaacagcctcaatcagaatgccaaaccagaggggcctgagcaggctgagctgggccgcctttccccacgagcctacacggccccacctgcctgccagccacccatggagcctgagaaccttgacctccagtccccaaccaagctgagtgccagcggggaggactccaccatcccacaagccagccggctcaataacatcgttaacaggtccatgacgggctctccccgcagcagcagcgagagccactcaccactctacatgcaccccccgaagtgcacgtcctgcggctctcagtccccacagcatgcagagatgtgcctccacaccgctggccccacgttccctgaggagatgggagagacccagtctgagtactcagattctagctgtgagaacggggccttcttctgcaatgagtgtgactgccgcttctctgaggaggcctcactcaagaggcacacgctgcagacccacagtgacaaaccctacaagtgtgaccgctgccaggcctccttccgctacaagggcaacctcgccagccacaagaccgtccataccggtgagaaaccctatcgttgcaacatctgtggggcccagttcaaccggccagccaacctgaaaacccacactcgaattcactctggagagaagccctacaaatgcgaaacctgcggagccagatttgtacaggtggcccacctccgtgcccatgtgcttatccacactggtgagaagccctatccctgtgaaatctgtggcacccgtttccggcaccttcagactctgaagcgccacctgcgaatccacacaggagagaaaccttaccattgtgagaagtgtaacctgcatttccgtcacaaaagccagctgcgacttcacttgcgccagaagcatggcgccatcaccaacaccaaggtgcaataccgcgtgtcagccactgacctgcctccggagctccccaaagcctgc

SEQ ID NO: 2 - protein, 706 aa, Synthetic construct
MASPADSCIQFTRHASDVLLNLNRLRSRDILTDVVIVVSREQFRAHKTVLMACSGLFYSIFTDQLKCNLSVINLDPEINPEGFCILLDFMYTSRLNLREGNIMAVMATAMYLQMEHVVDTCRKFIKASEAEMVSAIKPPREEFLNSRMLMPQDIMAYRGCEVVENNLPLRSAPGCESRAFAPSLYSGLSTPPASYSMYSHLPVSSLLFSDEEFRDVRMPVANPFPKERALPCDSARPVPGEYSRPTLEVSPNVCHSNIYSPKETIPEEARSDMHYSVAEGLKPAAPSARNAPYFPCDKASKEEERPSSEDEIALHFEPPNAPLNRKGLVSPQSPQKSDCQPNSPTESCSSKNACILQASGSPPAKSPTDPKACNWKKYKFIVLNSLNQNAKPEGPEQAELGRLSPRAYTAPPACQPPMEPENLDLQSPTKLSASGEDSTIPQASRLNNIVNRSMTGSPRSSSESHSPLYMHPPKCTSCGSQSPQHAEMCLHTAGPTFPEEMGETQSEYSDSSCENGAFFCNECDCRFSEEASLKRHTLQTHSDKPYKCDRCQASFRYKGNLASHKTVHTGEKPYRCNICGAQFNRPANLKTHTRIHSGEKPYKCETCGARFVQVAHLRAHVLIHTGEKPYPCEICGTRFRHLQTLKRHLRIHTGEKPYHCEKCNLHFRHKSQLRLHLRQKHGAITNTKVQYRVSATDLPPELPKAC

SEQ ID NO: 3 - DNA, 2118 bp, Homo sapiens
  source 1..2118: mol_type = genomic DNA
atggcctcgccggctgacagctgtatccagttcacccgccatgccagtgatgttcttctcaaccttaatcgtctccggagtcgagacatcttgactgatgttgtcattgttgtgagccgtgagcagtttagagcccataaaacggtcctcatggcctgcagtggcctgttctatagcatctttacagaccagttgaaatgcaaccttagtgtgatcaatctagatcctgagatcaaccctgagggattctgcatcctcctggacttcatgtacacatctcggctcaatttgcgggagggcaacatcatggctgtgatggccacggctatgtacctgcagatggagcatgttgtggacacttgccggaagtttattaaggccagtgaagcagagatggtttctgccatcaagcctcctcgtgaagagttcctcaacagccggatgctgatgccccaagacatcatggcctatcggggttgtgaggtggtggagaacaacctgccactgaggagcgcccctgggtgtgagagcagagcctttgcccccagcctgtacagtggcctgtccacaccgccagcctcttattccatgtacagccacctccctgtcagcagcctcctcttctccgatgaggagtttcgggatgtccggatgcctgtggccaaccccttccccaaggagcgggcactcccatgtgatagtgccaggccagtccctggtgagtacagccggccgactttggaggtgtcccccaatgtgtgccacagcaatatctattcacccaaggaaacaatcccagaagaggcacgaagtgatatgcactacagtgtggctgagggcctcaaacctgctgccccctcagcccgaaatgccccctacttcccttgtgacaaggccagcaaagaagaagagagaccctcctcggaagatgagattgccctgcatttcgagccccccaatgcacccctgaaccggaagggtctggttagtccacagagcccccagaaatctgactgccagcccaactcgcccacagagtcctgcagcagtaagaatgcctgcatcctccaggcttctggctcccctccagccaagagccccactgaccccaaagcctgcaactggaagaaatacaagttcatcgtgctcaacagcctcaatcagaatgccaaaccagaggggcctgagcaggctgagctgggccgcctttccccacgagcctacacggccccacctgcctgccagccacccatggagcctgagaaccttgacctccagtccccaaccaagctgagtgccagcggggaggactccaccatcccacaagccagccggctcaataacatcgttaacaggtccatgacgggctctccccgcagcagcagcgagagccactcaccactctacatgcaccccccgaagtgcacgtcctgcggctctcagtccccacagcatgcagagatgtgcctccacaccgctggccccacgttccctgaggagatgggagagacccagtctgagtactcagattctagctgtgagaacggggccttcttctgcaatgagtgtgactgccgcttctctgaggaggcctcactcaagaggcacacgctgcagacccacagtgacaaaccctacaagtgtgaccgctgccaggcctccttccgctacaagggcaacctcgccagccacaagaccgtccataccggtgagaaaccctatcgttgcaacatctgtggggcccagttcaaccggccagccaacctgaaaacccacactcgaattcactctggagagaagccctacaaatgcgaaacctgcggagccagatttgtacaggtggcccacctccgtgcccatgtgcttatccacactggtgagaagccctatccctgtgaaatctgtggcacccgtttccggcaccttcagactctgaagagccacctgcgaatccacacaggagagaaaccttaccattgtgagaagtgtaacctgcatttccgtcacaaaagccagctgcgacttcacttgcgccagaagcatggcgccatcaccaacaccaaggtgcaataccgcgtgtcagccactgacctgcctccggagctccccaaagcctgc

SEQ ID NO: 4 - protein, 706 aa, Homo sapiens [sequence omitted]

SEQ ID NO: 5 - DNA, 5265 bp, Synthetic construct
  source 1..5265: mol_type = other DNA
atgctctcagcaacccccctgtatgggaacgttcacagctggatgaacagcgagagggtccgcatgtgtggggcgagcgaagacaggaaaatccttgtaaatgatggtgacgcttcaaaagccagactggaactgagggaagagaatcccttgaaccacaacgtggtggatgcgagcacggcccataggatcgatggcctggcagcactgagcatggaccgcactggcctgatccgggaagggctgcgggtcccgggaaacatcgtctattctagcttgtgtggactgggctcagagaaaggtcgggaggctgccacaagcactctaggtggccttgggttttcttcggaaagaaatccagagatgcagttcaaaccgaatacacccgagacagtggaggcttctgccgtctctggaaaacccccaaatggcttcagtgctatatacaaaacaccgcctggaatacaaaaaagtgctgtagccacagcagaagcgctgggcttggacaggcctgccagcgacaaacagagccctctcaacatcaatggtgctagttatctgcggctgccctgggtcaatccttacatggagggtgccacgccagccatctaccctttcctcgactcgccaaataagtattcactgaacatgtacaaggccttgctacctcagcagtcctacagcttggcccagccgctgtattctccagtctgcaccaatggggagcgctttctctacctgccgccacctcactacgtcggtccccacatcccatcgtccttggcatcacccatgaggctctcgacaccttcggcctccccagccatcccgcctctcgtccattgcgcagacaaaagcctcccgtggaagatgggcgtcagccctgggaatcctgttgattcccacgcctatcctcacatccagaacagtaagcagcccagggttccctctgccaaggcggtcaccagtggcctgccgggggacacagctctcctgttgcccccctcgcctcggccgtcaccccgagtccaccttcccacccagcctgctgcagacacctactcggagttccacaagcactatgccaggatctccacctctccttcagttgccctgtcaaagccatacatgacagttagcagcgagttccccgcggccaggctctccaatggcaagtatcccaaggctccggaagggggcgaaggtgcccagccagtgcccgggcatgcccggaagacagcggttcaagacagaaaagatggcagctcacctcctctgttggagaagcagaccgttaccaaagacgtcacagataagccactagacttgtcttctaaagtggtggatgtagatgcttccaaagctgaccacatgaaaaagatggctcccacggtcctggttcacagcagggctggaagtggcttagtgctctccggaagtgagattccgaaagaaacactatctcctccaggaaatggttgtgctatctatagatctgaaatcatcagcactgctccctcatcctgggtggtgcccgggccaagtcctaacgaagagaacaatggcaaaagcatgtcgctgaaaaacaaggcattggactgggcgataccacagcagcggagttcatcatgcccgcgcatgggcggcaccgatgctgtcatcactaacgtttcagggtcagtgtcgagtgcaggccgcccagcctccgcatcacccgcccccaatgccaatgcagatggcaccaaaaccagcaggagctctgtagaaaccacaccatccgttattcagcacgtgggccagcccccggccactcctgccaagcacagtagcagcaccagcagcaagggcgccaaagccagcaacccagaaccgagtttcaaagcaaacgagaacggccttccaccaagctctatatttctgtctccaaatgaggcattcaggtccccaccaattccctaccccaggagttacctcccttacccagcccctgagggcattgctgtaagtcccctctccttacatggcaaaggacctgtctaccctcacccagttttgttacccaatggcagtctgtttcctgggcaccttgccccaaagcctgggctgccctatgggcttcccaccggccgtccagagtttgtgacctaccaagatgccctggggttgggcatggtgcatcccatgttgataccacacacgcccatagagattactaaagaggagaaaccagagaggagatcccggtcccatgagagagcccgttacgaggacccaaccctccggaatcggttttccgagattttggaaactagcagcaccaagttacatccagatgtccccaccgacaagaacctaaagccgaaccccaactggaatcaagggaagactgttgtcaaaagcgacaagcttgtctacgtagaccttctccgagaagaaccagatgctaaaactgacacaaacgtgtccaaacccagctttgcagcagagagtgttggccagagcgctgagccccccaagccctcagttgagccggccctgcagcagcaccgtgatttcatcgccctgagagaggagttggggcgcatcagtgacttccacgaaacttatactttcaaacagccagtcttcaccgtaagcaaggacagtgttctggcaggtaccaacaaagagaacctagggttgccagtctcgactccattcctggagccacctctggggagcgatggccctgctgtaacttttggtaaaacccaagaggatcccaaaccattttgtgtgggcagtgccccaccaagtgtggatgtgacccccacctataccaaagatggagctgatgaggctgaatcaaatgatggcaaagttctgaaaccgaagccatctaagctggcaaagagaatcgccaactcagcgggttacgtgggtgaccgattcaaatgtgtcactaccgaactgtatgcagattccagtcagctcagccgggagcaacgggcattgcagatggaaggattacaagaggacagtattttatgtctacccgctgcttactgtgagcgtgcaatgatgcgcttctcagagttggagatgaaagaaagagaaggtggccacccagcaaccaaagactccgagatgtgcaaattcagcccagccgactgggaaaggttgaaaggaaatcaggacaaaaagccaaagtcggtcaccctggaggaggccattgcagaacagaacgaaagtgagagatgcgagtatagtgttggaaacaagcaccgtgatccctttgaagccccagaggacaaagatcttcctgtggagaagtactttgtggagaggcagcctgtgagcgagcctcccgcagaccaggtggcctcggacatgcctcacagccccaccctccgggtggacaggaaacgcaaagtctcaggtgacagcagccacactgagaccactgcggaggaggtgccagaggaccctctgctgaaagccaaacgccgacgagtctctaaagatgactggcctgagagggaaatgacaaacagttcctctaaccacttagaagacccacattatagtgagctgaccaacctgaaggtgtgcattgaattaacagggctccatcctaaaaaacaacgccacttgctgcaccttagagaacgatgggagcagcaggtgtcggcagcagatggcaaacctggccggcaaagcaggaaggaagtgacccaggccactcagcctgaggccattcctcaggggactaacatcactgaagagaaacctggcaggaaaagggcagaggccaaaggcaacagaagctggtcggaagagtctcttaaacccagtgacaatgaacaaggcttgcctgtgttctccggctctccgcccatgaagagtctttcatccaccagtgcaggcggcaaaaagcaggctcagccaagctgcgcaccagcctccaggccgcctgccaaacagcagaaaattaaagaaaaccagaagacagatgtgctgtgtgcagacgaagaagaggattgccaggctgcctccctgctgcagaaatacaccgacaacagcgagaagccatccgggaagagactgtgcaaaaccaaacacttgatccctcaggagtccaggcggggattgccactgacaggggaatactacgtggagaatgccgatggcaaggtgactgtccggagattcagaaagcggccggagcccagttcggactatgatctgtcaccagccaagcaggagccaaagcccttcgaccgcttgcagcaactgctaccagcctcccagtccacacagctgccatgctcaagttcccctcaggagaccacccagtctcgccctatgccgccggaagcacggagacttattgtctctaagaacgctggcgagacccttctgcagcgggcagccaggcttggctatgaggaagtggtcctgtactgcttagagaacaagatttgtgatgtaaatcatcgggacaacgcaggttactgcgccctgcatgaagcttgtgctaggggctggctcaacattgtgcgacacctccttgaatatggcgctgatgtcaactgtagtgcccaggatggaaccaggcctctgcacgatgctgttgagaacgatcacttggaaattgtccgactacttctctcttatggtgctgaccccaccttggctacgtactcaggtagaaccatcatgaaaatgacccacagtgaacttatggaaaagttcttaacagattatttaaatgacctccagggtcgcaatgatgatgacgccagtggcacttgggacttctatggcagctctgtttgtgaaccagatgatgaaagtggctatgatgttttagccaaccccccaggaccagaagaccaggatgatgatgacgatgcctatagcgatgtgtttgaatttgaattttcagagacccccctcttaccgtgttataacatccaagtatctgtggctcaggggccacgaaactggctactgctttcggatgtccttaagaaattgaaaatgtcctcccgcatatttcgctgcaattttccaaacgtggaaattgtcaccattgcagaggcagaattttatcggcaggtttctgcaagtctcttgttctcttgctccaaagacctggaagccttcaaccctgaaagtaaggagctgttagatctggtggaattcacgaacgaaattcagactctgctgggctcctctgtagagtggctccaccccagtgatctggcctcagacaactactgg

SEQ ID NO: 6 - protein, 1755 aa, Synthetic construct
MLSATPLYGNVHSWMNSERVRMCGASEDRKILVNDGDASKARLELREENPLNHNVVDASTAHRIDGLAALSMDRTGLIREGLRVPGNIVYSSLCGLGSEKGREAATSTLGGLGFSSERNPEMQFKPNTPETVEASAVSGKPPNGFSAIYKTPPGIQKSAVATAEALGLDRPASDKQSPLNINGASYLRLPWVNPYMEGATPAIYPFLDSPNKYSLNMYKALLPQQSYSLAQPLYSPVCTNGERFLYLPPPHYVGPHIPSSLASPMRLSTPSASPAIPPLVHCADKSLPWKMGVSPGNPVDSHAYPHIQNSKQPRVPSAKAVTSGLPGDTALLLPPSPRPSPRVHLPTQPAADTYSEFHKHYARISTSPSVALSKPYMTVSSEFPAARLSNGKYPKAPEGGEGAQPVPGHARKTAVQDRKDGSSPPLLEKQTVTKDVTDKPLDLSSKVVDVDASKADHMKKMAPTVLVHSRAGSGLVLSGSEIPKETLSPPGNGCAIYRSEIISTAPSSWVVPGPSPNEENNGKSMSLKNKALDWAIPQQRSSSCPRMGGTDAVITNVSGSVSSAGRPASASPAPNANADGTKTSRSSVETTPSVIQHVGQPPATPAKHSSSTSSKGAKASNPEPSFKANENGLPPSSIFLSPNEAFRSPPIPYPRSYLPYPAPEGIAVSPLSLHGKGPVYPHPVLLPNGSLFPGHLAPKPGLPYGLPTGRPEFVTYQDALGLGMVHPMLIPHTPIEITKEEKPERRSRSHERARYEDPTLRNRFSEILETSSTKLHPDVPTDKNLKPNPNWNQGKTVVKSDKLVYVDLLREEPDAKTDTNVSKPSFAAESVGQSAEPPKPSVEPALQQHRDFIALREELGRISDFHETYTFKQPVFTVSKDSVLAGTNKENLGLPVSTPFLEPPLGSDGPAVTFGKTQEDPKPFCVGSAPPSVDVTPTYTKDGADEAESNDGKVLKPKPSKLAKRIANSAGYVGDRFKCVTTELYADSSQLSREQRALQMEGLQEDSILCLPAAYCERAMMRFSELEMKEREGGHPATKDSEMCKFSPADWERLKGNQDKKPKSVTLEEAIAEQNESERCEYSVGNKHRDPFEAPEDKDLPVEKYFVERQPVSEPPADQVASDMPHSPTLRVDRKRKVSGDSSHTETTAEEVPEDPLLKAKRRRVSKDDWPEREMTNSSSNHLEDPHYSELTNLKVCIELTGLHPKKQRHLLHLRERWEQQVSAADGKPGRQSRKEVTQATQPEAIPQGTNITEEKPGRKRAEAKGNRSWSEESLKPSDNEQGLPVFSGSPPMKSLSSTSAGGKKQAQPSCAPASRPPAKQQKIKENQKTDVLCADEEEDCQAASLLQKYTDNSEKPSGKRLCKTKHLIPQESRRGLPLTGEYYVENADGKVTVRRFRKRPEPSSDYDLSPAKQEPKPFDRLQQLLPASQSTQLPCSSSPQETTQSRPMPPEARRLIVSKNAGETLLQRAARLGYEEVVLYCLENKICDVNHRDNAGYCALHEACARGWLNIVRHLLEYGADVNCSAQDGTRPLHDAVENDHLEIVRLLLSYGADPTLATYSGRTIMKMTHSELMEKFLTDYLNDLQGRNDDDASGTWDFYGSSVCEPDDESGYDVLANPPGPEDQDDDDDAYSDVFEFEFSETPLLPCYNIQVSVAQGPRNWLLLSDVLKKLKMSSRIFRCNFPNVEIVTIAEAEFYRQVSASLLFSCSKDLEAFNPESKELLDLVEFTNEIQTLLGSSVEWLHPSDLASDNYW

SEQ ID NO: 7 - DNA, 5265 bp, Homo sapiens
  source 1..5265: mol_type = genomic DNA
atgctctcagcaacccccctgtatgggaacgttcacagctggatgaacagcgagagggtccgcatgtgtggggcgagcgaagacaggaaaatccttgtaaatgatggtgacgcttcaaaagccagactggaactgagggaagagaatcccttgaaccacaacgtggtggatgcgagcacggcccataggatcgatggcctggcagcactgagcatggaccgcactggcctgatccgggaagggctgcgggtcccgggaaacatcgtctattctagcttgtgtggactgggctcagagaaaggtcgggaggctgccacaagcactctaggtggccttgggttttcttcggaaagaaatccagagatgcagttcaaaccgaatacacccgagacagtggaggcttctgccgtctctggaaaacccccaaatggcttcagtgctatatacaaaacaccgcctggaatacaaaaaagtgctgtagccacagcagaagcgctgggcttggacaggcctgccagcgacaaacagagccctctcaacatcaatggtgctagttatctgcggctgccctgggtcaatccttacatggagggtgccacgccagccatctaccctttcctcgactcgccaaataagtattcactgaacatgtacaaggccttgctacctcagcagtcctacagcttggcccagccgctgtattctccagtctgcaccaatggggagcgctttctctacctgccgccacctcactacgtcggtccccacatcccatcgtccttggcatcacccatgaggctctcgacaccttcggcctccccagccatcccgcctctcgtccattgcgcagacaaaagcctcccgtggaagatgggcgtcagccctgggaatcctgttgattcccacgcctatcctcacatccagaacagtaagcagcccagggttccctctgccaaggcggtcaccagtggcctgccgggggacacagctctcctgttgcccccctcgcctcggccgtcaccccgagtccaccttcccacccagcctgctgcagacacctactcggagttccacaagcactatgccaggatctccacctctccttcagttgccctgtcaaagccatacatgacagttagcagcgagttccccgcggccaggctctccaatggcaagtatcccaaggctccggaagggggcgaaggtgcccagccagtgcccgggcatgcccggaagacagcggttcaagacagaaaagatggcagctcacctcctctgttggagaagcagaccgttaccaaagacgtcacagataagccactagacttgtcttctaaagtggtggatgtagatgcttccaaagctgaccacatgaaaaagatggctcccacggtcctggttcacagcagggctggaagtggcttagtgctctccggaagtgagattccgaaagaaacactatctcctccaggaaatggttgtgctatctatagatctgaaatcatcagcactgctccctcatcctgggtggtgcccgggccaagtcctaacgaagagaacaatggcaaaagcatgtcgctgaaaaacaaggcattggactgggcgataccacagcagcggagttcatcatgcccgcgcatgggcggcaccgatgctgtcatcactaacgtttcagggtcagtgtcgagtgcaggccgcccagcctccgcatcacccgcccccaatgccaatgcagatggcaccaaaaccagcaggagctctgtagaaaccacaccatccgttattcagcacgtgggccagcccccggccactcctgccaagcacagtagcagcaccagcagcaagggcgccaaagccagcaacccagaaccgagtttcaaagcaaacgagaacggccttccaccaagctctatatttctgtctccaaatgaggcattcaggtccccaccaattccctaccccaggagttacctcccttacccagcccctgagggcattgctgtaagtcccctctccttacatggcaaaggacctgtctaccctcacccagttttgttacccaatggcagtctgtttcctgggcaccttgccccaaagcctgggctgccctatgggcttcccaccggccgtccagagtttgtgacctaccaagatgccctggggttgggcatggtgcatcccatgttgataccacacacgcccatagagattactaaagaggagaaaccagagaggagatcccggtcccatgagagagcccgttacgaggacccaaccctccggaatcggttttccgagattttggaaactagcagcaccaagttacatccagatgtccccaccgacaagaacctaaagccgaaccccaactggaatcaagggaagactgttgtcaaaagcgacaagcttgtctacgtagaccttctccgagaagaaccagatgctaaaactgacacaaacgtgtccaaacccagctttgcagcagagagtgttggccagagcgctgagccccccaagccctcagttgagccggccctgcagcagcaccgtgatttcatcgccctgagagaggagttggggcgcatcagtgacttccacgaaacttatactttcaaacagccagtcttcaccgtaagcaaggacagtgttctggcaggtaccaacaaagagaacctagggttgccagtctcgactccattcctggagccacctctggggagcgatggccctgctgtaacttttggtaaaacccaagaggatcccaaaccattttgtgtgggcagtgccccaccaagtgtggatgtgacccccacctataccaaagatggagctgatgaggctgaatcaaatgatggcaaagttctgaaaccgaagccatctaagctggcaaagagaatcgccaactcagcgggttacgtgggtgaccgattcaaatgtgtcactaccgaactgtatgcagattccagtcagctcagccgggagcaacgggcattgcagatggaaggattacaagaggacagtattttatgtctacccgctgcttactgtgagcgtgcaatgatgcgcttctcagagttggagatgaaagaaagagaaggtggccacccagcaaccaaagactccgagatgtgcaaattcagcccagccgactgggaaaggttgaaaggaaatcaggacaaaaagccaaagtcggtcaccctggaggaggccattgcagaacagaacgaaagtgagagatgcgagtatagtgttggaaacaagcaccgtgatccctttgaagccccagaggacaaagatcttcctgtggagaagtactttgtggagaggcagcctgtgagcgagcctcccgcagaccaggtggcctcggacatgcctcacagccccaccctccgggtggacaggaaacgcaaagtctcaggtgacagcagccacactgagaccactgcggaggaggtgccagaggaccctctgctgaaagccaaacgccgacgagtctctaaagatgactggcctgagagggaaatgacaaacagttcctctaaccacttagaagacccacattatagtgagctgaccaacctgaaggtgtgcattgaattaacagggctccatcctaaaaaacaacgccacttgctgcaccttagagaacgatgggagcagcaggtgtcggcagcagatggcaaacctggccggcaaagcaggaaggaagtgacccaggccactcagcctgaggccattcctcaggggactaacatcactgaagagaaacctggcaggaaaagggcagaggccaaaggcaacagaagctggtcggaagagtctcttaaacccagtgacaatgaacaaggcttgcctgtgttctccggctctccgcccatgaagagtctttcatccaccagtgcaggcggcaaaaagcaggctcagccaagctgcgcaccagcctccaggccgcctgccaaacagcagaaaattaaagaaaaccagaagacagatgtgctgtgtgcagacgaagaagaggattgccaggctgcctccctgctgcagaaatacaccgacaacagcgagaagccatccgggaagagactgtgcaaaaccaaacacttgatccctcaggagtccaggcggggattgccactgacaggggaatactacgtggagaatgccgatggcaaggtgactgtccggagattcagaaagcggccggagcccagttcggactatgatctgtcaccagccaagcaggagccaaagcccttcgaccgcttgcagcaactgctaccagcctcccagtccacacagctgccatgctcaagttcccctcaggagaccacccagtctcgccctatgccgccggaagcacggagacttattgtcaataagaacgctggcgagacccttctgcagcgggcagccaggcttggctatgaggaagtggtcctgtactgcttagagaacaagatttgtgatgtaaatcatcgggacaacgcaggttactgcgccctgcatgaagcttgtgctaggggctggctcaacattgtgcgacacctccttgaatatggcgctgatgtcaactgtagtgcccaggatggaaccaggcctctgcacgatgctgttgagaacgatcacttggaaattgtccgactacttctctcttatggtgctgaccccaccttggctacgtactcaggtagaaccatcatgaaaatgacccacagtgaacttatggaaaagttcttaacagattatttaaatgacctccagggtcgcaatgatgatgacgccagtggcacttgggacttctatggcagctctgtttgtgaaccagatgatgaaagtggctatgatgttttagccaaccccccaggaccagaagaccaggatgatgatgacgatgcctatagcgatgtgtttgaatttgaattttcagagacccccctcttaccgtgttataacatccaagtatctgtggctcaggggccacgaaactggctactgctttcggatgtccttaagaaattgaaaatgtcctcccgcatatttcgctgcaattttccaaacgtggaaattgtcaccattgcagaggcagaattttatcggcaggtttctgcaagtctcttgttctcttgctccaaagacctggaagccttcaaccctgaaagtaaggagctgttagatctggtggaattcacgaacgaaattcagactctgctgggctcctctgtagagtggctccaccccagtgatctggcctcagacaactactgg

SEQ ID NO: 8 - protein, 1755 aa, Homo sapiens
MLSATPLYGNVHSWMNSERVRMCGASEDRKILVNDGDASKARLELREENPLNHNVVDASTAHRIDGLAALSMDRTGLIREGLRVPGNIVYSSLCGLGSEKGREAATSTLGGLGFSSERNPEMQFKPNTPETVEASAVSGKPPNGFSAIYKTPPGIQKSAVATAEALGLDRPASDKQSPLNINGASYLRLPWVNPYMEGATPAIYPFLDSPNKYSLNMYKALLPQQSYSLAQPLYSPVCTNGERFLYLPPPHYVGPHIPSSLASPMRLSTPSASPAIPPLVHCADKSLPWKMGVSPGNPVDSHAYPHIQNSKQPRVPSAKAVTSGLPGDTALLLPPSPRPSPRVHLPTQPAADTYSEFHKHYARISTSPSVALSKPYMTVSSEFPAARLSNGKYPKAPEGGEGAQPVPGHARKTAVQDRKDGSSPPLLEKQTVTKDVTDKPLDLSSKVVDVDASKADHMKKMAPTVLVHSRAGSGLVLSGSEIPKETLSPPGNGCAIYRSEIISTAPSSWVVPGPSPNEENNGKSMSLKNKALDWAIPQQRSSSCPRMGGTDAVITNVSGSVSSAGRPASASPAPNANADGTKTSRSSVETTPSVIQHVGQPPATPAKHSSSTSSKGAKASNPEPSFKANENGLPPSSIFLSPNEAFRSPPIPYPRSYLPYPAPEGIAVSPLSLHGKGPVYPHPVLLPNGSLFPGHLAPKPGLPYGLPTGRPEFVTYQDALGLGMVHPMLIPHTPIEITKEEKPERRSRSHERARYEDPTLRNRFSEILETSSTKLHPDVPTDKNLKPNPNWNQGKTVVKSDKLVYVDLLREEPDAKTDTNVSKPSFAAESVGQSAEPPKPSVEPALQQHRDFIALREELGRISDFHETYTFKQPVFTVSKDSVLAGTNKENLGLPVSTPFLEPPLGSDGPAVTFGKTQEDPKPFCVGSAPPSVDVTPTYTKDGADEAESNDGKVLKPKPSKLAKRIANSAGYVGDRFKCVTTELYADSSQLSREQRALQMEGLQEDSILCLPAAYCERAMMRFSELEMKEREGGHPATKDSEMCKFSPADWERLKGNQDKKPKSVTLEEAIAEQNESERCEYSVGNKHRDPFEAPEDKDLPVEKYFVERQPVSEPPADQVASDMPHSPTLRVDRKRKVSGDSSHTETTAEEVPEDPLLKAKRRRVSKDDWPEREMTNSSSNHLEDPHYSELTNLKVCIELTGLHPKKQRHLLHLRERWEQQVSAADGKPGRQSRKEVTQATQPEAIPQGTNITEEKPGRKRAEAKGNRSWSEESLKPSDNEQGLPVFSGSPPMKSLSSTSAGGKKQAQPSCAPASRPPAKQQKIKENQKTDVLCADEEEDCQAASLLQKYTDNSEKPSGKRLCKTKHLIPQESRRGLPLTGEYYVENADGKVTVRRFRKRPEPSSDYDLSPAKQEPKPFDRLQQLLPASQSTQLPCSSSPQETTQSRPMPPEARRLIVNKNAGETLLQRAARLGYEEVVLYCLENKICDVNHRDNAGYCALHEACARGWLNIVRHLLEYGADVNCSAQDGTRPLHDAVENDHLEIVRLLLSYGADPTLATYSGRTIMKMTHSELMEKFLTDYLNDLQGRNDDDASGTWDFYGSSVCEPDDESGYDVLANPPGPEDQDDDDDAYSDVFEFEFSETPLLPCYNIQVSVAQGPRNWLLLSDVLKKLKMSSRIFRCNFPNVEIVTIAEAEFYRQVSASLLFSCSKDLEAFNPESKELLDLVEFTNEIQTLLGSSVEWLHPSDLASDNYW

SEQ ID NO: 9 - DNA, 2298 bp, Synthetic construct
  source 1..2298: mol_type = other DNA
atggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctcccgggcctcagcgagaaaggaagtcatcttcatcctcagaagacaggaatcgaatgaaaacacttggtagacgggactcgagtgatgattgggagattcctgatgggcagattacagtgggacaaagaattggatctggatcatttcgaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaagcttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctgggtccattttgtggatggcaccagaagtcatcagaatgcaagataaaaatccatacagctttcagtcagatgtatatgcatttgggattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccac

SEQ ID NO: 10 - protein, 766 aa, Synthetic construct
MAALSGGGGGGAEPGQALFNGDMEPEAGAGAGAAASSAADPAIPEEVWNIKQMIKLTQEHIEALLDKFGGEHNPPSIYLEAYEEYTSKLDALQQREQQLLESLGNGTDFSVSSSASMDTVTSSSSSSLSVLPSSLSVFQNPTDVARSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETALTSGSSPSAPASDSIGPQILTSPSPSKSIPIPQPFRPADEDHRNQFGQRDRSSSAPNVHINTIEPVNIDDLIRDQGFRGDGGSTTGLSATPPASLPGSLTNVKALQKSPGPQRERKSSSSSEDRNRMKTLGRRDSSDDWEIPDGQITVGQRIGSGSFRTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGAFPVH

SEQ ID NO: 11 - DNA, 2298 bp, Synthetic construct
  source 1..2298: mol_type = other DNA
atggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctcccgggcctcagcgagaaaggaagtcatcttcatcctcagaagacaggaatcgaatgaaaacacttggtagacgggactcgagtgatgattgggagattcctgatgggcagattacagtgggacaaagaattggatctggatcatttgcaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaagcttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctgggtccattttgtggatggcaccagaagtcatcagaatgcaagataaaaatccatacagctttcagtcagatgtatatgcatttgggattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccac

SEQ ID NO: 12 - protein, 766 aa, Synthetic construct
MAALSGGGGGGAEPGQALFNGDMEPEAGAGAGAAASSAADPAIPEEVWNIKQMIKLTQEHIEALLDKFGGEHNPPSIYLEAYEEYTSKLDALQQREQQLLESLGNGTDFSVSSSASMDTVTSSSSSSLSVLPSSLSVFQNPTDVARSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETALTSGSSPSAPASDSIGPQILTSPSPSKSIPIPQPFRPADEDHRNQFGQRDRSSSAPNVHINTIEPVNIDDLIRDQGFRGDGGSTTGLSATPPASLPGSLTNVKALQKSPGPQRERKSSSSSEDRNRMKTLGRRDSSDDWEIPDGQITVGQRIGSGSFATVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGAFPVH

SEQ ID NO: 13 - DNA, 2298 bp, Synthetic construct
  source 1..2298: mol_type = other DNA
atggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctcccgggcctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaagcttatagatattgcacgacagactgcacagggcatggattacttacacgccaagtcaatcatccacagagacctcaagagtaataatatatttcttcatgaagacctcacagtaaaaataggtaattttggtctagctacagtgaaatctcgatggagtgggtcccatcagtttgaacagttgtctgggtccattttgtggatggcaccagaagtcatcagaatgcaagataaaaatccatacagctttcagtcagatgtatatgcatttgggattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccac

SEQ ID NO: 14 - protein, 766 aa, Synthetic construct
MAALSGGGGGGAEPGQALFNGDMEPEAGAGAGAAASSAADPAIPEEVWNIKQMIKLTQEHIEALLDKFGGEHNPPSIYLEAYEEYTSKLDALQQREQQLLESLGNGTDFSVSSSASMDTVTSSSSSSLSVLPSSLSVFQNPTDVARSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETALTSGSSPSAPASDSIGPQILTSPSPSKSIPIPQPFRPADEDHRNQFGQRDRSSSAPNVHINTIEPVNIDDLIRDQGFRGDGGSTTGLSATPPASLPGSLTNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGNFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGAFPVH

SEQ ID NO: 15 - DNA, 2298 bp, Homo sapiens
  source 1..2298: mol_type = genomic DNA
atggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctcccgggcctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaagcttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctgggtccattttgtggatggcaccagaagtcatcagaatgcaagataaaaatccatacagctttcagtcagatgtatatgcatttgggattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccac

SEQ ID NO: 16 - protein, 766 aa, Homo sapiens [sequence omitted]

SEQ ID NO: 17 - DNA, 3441 bp, Synthetic construct
  source 1..3441: mol_type = other DNA
atggatgactacatggagacgctgaaggatgaagaggacgccttgtgggagaatgtggagtgtaaccggcacatgctcagccgctatatcaaccctgccaagctcacgccctacctgcgtcagtgtaaggtcattgatgagcaggatgaagatgaagtgcttaatgcccctatgctgccatccaagatcaaccgagcaggccggctgttggacattctacataccaaggggcaaaggggctatgtggtcttcttggagagcctagaattttattacccagaactgtacaaactggtgactgggaaagagcccactcggagattctccaccattgtggtggaggaaggccacgagggcctcacgcacttcctgatgaacgaggtcatcaagctgcagcagcagatgaaggccaaggacctgcaacgctgcgagctgctggccaggttgcggcagctggaggatgagaagaagcagatgacgctgacgcgcgtggagctgctaaccttccaggagcggtactacaagatgaaggaagagcgggacagctacaatgacgagctggtcaaggtgaaggacgacaactacaacttagccatgcgctacgcacagctcagtgaggagaagaacatggcggtcatgaggagccgagacctccaactcgagatcgatcagctaaagcaccggttgaataagatggaggaggaatgtaagctggagagaaatcagtctctaaaactgaagaatgacattgaaaatcggcccaagaaggagcaggttctggaactggagcgggagaatgaaatgctgaagaccaaaaaccaggagctgcagtccatcatccaggccgggaagcgcagcctgccagactcagacaaggccatcctggacatcttggaacacgaccgcaaggaggccctggaggacaggcaggagctggtcaacaggatctacaacctgcaggaggaggcccgccaggcagaggagctgcgagacaagtacctggaggagaaggaggacctggagctcaagtgctcgaccctgggaaagaactgtgaaatgtacaagcaccgcatgaacacggtcatgctgcagctggaggaggtggagcgggagcgggaccaggccttccactcccgagatgaagctcagacacagtactcgcagtgcttaatcgaaaaggacaagtacaggaagcagatccgcgagctggaggagaagaacgatgagatgaggatcgagatggtgcggcgggaggcctgcatcgtcaacctggagagcaagctgcggcgcctctccaaggacagcaacaacctggaccagagtctgcccaggaacctgccagtaaccatcatctctcaggactttggggatgccagccccaggaccaatggtcaagaagctgacgattcttccacctcggaggagtcacctgaagacagcaagtacttcctgccctaccatccgccccagcgcaggatgaacctgaagggcatccagctgcagagagccaaatcccccatcagcctgaagcgaacatcagattttcaagccaaggggcacgaggaagaaggcacggatgccagccctagctcctgcggatctctgcccatcaccaactccttcaccaagatgcagcccccccggagccgcagcagcatcatgtcaatcaccgccgagcccccgggaaacgactccatcgtcagacgctacaaggaggacgcgccccatcgcagcacagtcgaagaagacaatgacagcggcgggtttgacgccttagatctggatgatgacagtcacgaacgctactccttcggaccctcctccatccactcctcctcctcctcccaccaatccgagggcctggatgcctacgacctggagcaggtcaacctcatgttcaggaagttctctctggaaagacccttccggccttcggtcacctctgtggggcacgtgcggggcccagggccctcggtgcagcacacgacgctgaatggcgacagcctcacctcccagctcaccctgctggggggcaacgcgcgagggagcttcgtgcactcggtcaagcctggctctctggccgagaaagccggcctccgtgagggccaccagctgctgctgctagaaggctgcatccgaggcgagaggcagagtgtcccgttggacacatgcaccaaagaggaagcccactggaccatccagaggtgcagcggccccgtcacgctgcactacaaggtcaaccacgaagggtaccggaagctggtgaaggacatggaggacggcctgatcacatcgggggactcgttctacatccggctgaacctgaacatctccagccagctggacgcctgcaccatgtccctgaagtgtgacgatgttgtgcacgtccgtgacaccatgtaccaggacaggcacgagtggctgtgcgcgcgggtcgaccctttcacagaccatgacctggatatgggcaccatacccagctacagccgagcccagcagctcctcctggtgaaactgcagcgcctgatgcaccgaggcagccgggaggaggtagacggcacccaccacaccctgcgggcactccggaacaccctgcagccagaagaagcgctttcaacaagcgacccccgggtcagcccccgtctctcgcgagcaagcttcctttttggccagctccttcagttcgtcagcaggtccgagaacaagtataagcggatgaacagcaacgagcgggtccgcatcatctcggggagtccgctagggagcctggcccggtcctcgctggacgccaccaagctcttgactgagaagcaggaagagctggaccctgagagcgagctgggcaagaacctcagcctcatcccctacagcctggtacgcgccttctactgcgagcgccgccggcccgtgctcttcacacccaccgtgctggccaagacgctggtgcagaggctgctcaactcgggaggtgccatggagttcaccatctgcaagtcagatatcgtcacaagagatgagttcctcagaaggcagaagacggagaccatcatctactcccgagagaagaaccccaacgcgttcgaatgcatcgcccctgccaacattgaagctgtggccgccaagaacaagcactgcctgctggaggctgggatcggctgcacaagagacttgatcaagtccaacatctaccccatcgtgctcttcatccgggtgtgtgagaagaacatcaagaggttcagaaagctgctgccccgacctgagacggaggaggagttcctgcgcgtgtgccggctgaaggagaaggagctggaggccctgccgtgcctgtacgccacggtggaacctgacatgtggggcagcgtagaggagctgctccgcgttgtcaaggacaagatcggcgaggagcagcgcaagaccatctgggtggacgaggaccagctg

SEQ ID NO: 18 - protein, 1147 aa, Synthetic construct
MDDYMETLKDEEDALWENVECNRHMLSRYINPAKLTPYLRQCKVIDEQDEDEVLNAPMLPSKINRAGRLLDILHTKGQRGYVVFLESLEFYYPELYKLVTGKEPTRRFSTIVVEEGHEGLTHFLMNEVIKLQQQMKAKDLQRCELLARLRQLEDEKKQMTLTRVELLTFQERYYKMKEERDSYNDELVKVKDDNYNLAMRYAQLSEEKNMAVMRSRDLQLEIDQLKHRLNKMEEECKLERNQSLKLKNDIENRPKKEQVLELERENEMLKTKNQELQSIIQAGKRSLPDSDKAILDILEHDRKEALEDRQELVNRIYNLQEEARQAEELRDKYLEEKEDLELKCSTLGKNCEMYKHRMNTVMLQLEEVERERDQAFHSRDEAQTQYSQCLIEKDKYRKQIRELEEKNDEMRIEMVRREACIVNLESKLRRLSKDSNNLDQSLPRNLPVTIISQDFGDASPRTNGQEADDSSTSEESPEDSKYFLPYHPPQRRMNLKGIQLQRAKSPISLKRTSDFQAKGHEEEGTDASPSSCGSLPITNSFTKMQPPRSRSSIMSITAEPPGNDSIVRRYKEDAPHRSTVEEDNDSGGFDALDLDDDSHERYSFGPSSIHSSSSSHQSEGLDAYDLEQVNLMFRKFSLERPFRPSVTSVGHVRGPGPSVQHTTLNGDSLTSQLTLLGGNARGSFVHSVKPGSLAEKAGLREGHQLLLLEGCIRGERQSVPLDTCTKEEAHWTIQRCSGPVTLHYKVNHEGYRKLVKDMEDGLITSGDSFYIRLNLNISSQLDACTMSLKCDDVVHVRDTMYQDRHEWLCARVDPFTDHDLDMGTIPSYSRAQQLLLVKLQRLMHRGSREEVDGTHHTLRALRNTLQPEEALSTSDPRVSPRLSRASFLFGQLLQFVSRSENKYKRMNSNERVRIISGSPLGSLARSSLDATKLLTEKQEELDPESELGKNLSLIPYSLVRAFYCERRRPVLFTPTVLAKTLVQRLLNSGGAMEFTICKSDIVTRDEFLRRQKTETIIYSREKNPNAFECIAPANIEAVAAKNKHCLLEAGIGCTRDLIKSNIYPIVLFIRVCEKNIKRFRKLLPRPETEEEFLRVCRLKEKELEALPCLYATVEPDMWGSVEELLRVVKDKIGEEQRKTIWVDEDQL

SEQ ID NO: 19 - DNA, 3441 bp, Synthetic construct
  source 1..3441: mol_type = other DNA
atggatgactacatggagacgctgaaggatgaagaggacgccttgtgggagaatgtggagtgtaaccggcacatgctcagccgctatatcaaccctgccaagctcacgccctacctgcgtcagtgtaaggtcattgatgagcaggatgaagatgaagtgcttaatgcccctatgctgccatccaagatcaaccgagcaggccggctgttggacattctacataccaaggggcaaaggggctatgtggtcttcttggagagcctagaattttattacccagaactgtacaaactggtgactgggaaagagcccactcggagattctccaccattgtggtggaggaaggccacgagggcctcacgcacttcctgatgaacgaggtcatcaagctgcagcagcagatgaaggccaaggacctgcaacgctgcgagctgctggccaggttgcggcagctggaggatgagaagaagcagatgacgctgacgcgcgtggagctgctaaccttccaggagcggtactacaagatgaaggaagagcgggacagctacaatgacgagctggtcaaggtgaaggacgacaactacaacttagccatgcgctacgcacagctcagtgaggagaagaacatggcggtcatgaggagccgagacctccaactcgagatcgatcagctaaagcaccggttgaataagatggaggaggaatgtaagctggagagaaatcagtctctaaaactgaagaatgacattgaaaatcggcccaagaaggagcaggttctggaactggagcgggagaatgaaatgctgaagaccaaaaaccaggagctgcagtccatcatccaggccgggaagcgcagcctgccagactcagacaaggccatcctggacatcttggaacacgaccgcaaggaggccctggaggacaggcaggagctggtcaacaggatctacaacctgcaggaggaggcccgccaggcagaggagctgcgagacaagtacctggaggagaaggaggacctggagctcaagtgctcgaccctgggaaaggactgtgaaatgtacaagcaccgcatgaacacggtcatgctgcagctggaggaggtggagcgggagcgggaccaggccttccactcccgagatgaagctcagacacagtactcgcagtgcttaatcgaaaaggacaagtacaggaagcagatccgcgagctggaggagaagaacgatgagatgaggatcgagatggtgcggcgggaggcctgcatcgtcaacctggagagcaagctgcggcgcctctccaaggacagcaacaacctggaccagagtctgcccaggaacctgccagtaaccatcatctctcaggactttggggatgccagccccaggaccaatggtcaagaagctgacgattcttccacctcggaggagtcacctgaagacagcaagtacttcctgccctaccatccgccccagcgcaggatgaacctgaagggcatccagctgcagagagccaaatcccccatcagcctgaagcgaacatcagattttcaagccaaggggcacgaggaagaaggcacggatgccagccctagctcctgcggatctctgcccatcaccaactccttcaccaagatgcagcccccccggagccgcagcagcatcatgtcaatcaccgccgagcccccgggaaacgactccatcgtcagacgctacaaggaggacgcgccccatcgcagcacagtcgaagaagacaatgacagcggcgggtttgacgccttagatctggatgatgacagtcacgaacgctactccttcggaccctccttcatccactcctcctcctcctcccaccaatccgagggcctggatgcctacgacctggagcaggtcaacctcatgttcaggaagttctctctggaaagacccttccggccttcggtcacctctgtggggcacgtgcggggcccagggccctcggtgcagcacacgacgctgaatggcgacagcctcacctcccagctcaccctgctggggggcaacgcgcgagggagcttcgtgcactcggtcaagcctggctctctggccgagaaagccggcctccgtgagggccaccagctgctgctgctagaaggctgcatccgaggcgagaggcagagtgtcccgttggacacatgcaccaaagaggaagcccactggaccatccagaggtgcagcggccccgtcacgctgcactacaaggtcaaccacgaagggtaccggaagctggtgaaggacatggaggacggcctgatcacatcgggggactcgttctacatccggctgaacctgaacatctccagccagctggacgcctgcaccatgtccctgaagtgtgacgatgttgtgcacgtccgtgacaccatgtaccaggacaggcacgagtggctgtgcgcgcgggtcgaccctttcacagaccatgacctggatatgggcaccatacccagctacagccgagcccagcagctcctcctggtgaaactgcagcgcctgatgcaccgaggcagccgggaggaggtagacggcacccaccacaccctgcgggcactccggaacaccctgcagccagaagaagcgctttcaacaagcgacccccgggtcagcccccgtctctcgcgagcaagcttcctttttggccagctccttcagttcgtcagcaggtccgagaacaagtataagcggatgaacagcaacgagcgggtccgcatcatctcggggagtccgctagggagcctggcccggtcctcgctggacgccaccaagctcttgactgagaagcaggaagagctggaccctgagagcgagctgggcaagaacctcagcctcatcccctacagcctggtacgcgccttctactgcgagcgccgccggcccgtgctcttcacacccaccgtgctggccaagacgctggtgcagaggctgctcaactcgggaggtgccatggagttcaccatctgcaagtcagatatcgtcacaagagatgagttcctcagaaggcagaagacggagaccatcatctactcccgagagaagaaccccaacgcgttcgaatgcatcgcccctgccaacattgaagctgtggccgccaagaacaagcactgcctgctggaggctgggatcggctgcacaagagacttgatcaagtccaacatctaccccatcgtgctcttcatccgggtgtgtgagaagaacatcaagaggttcagaaagctgctgccccgacctgagacggaggaggagttcctgcgcgtgtgccggctgaaggagaaggagctggaggccctgccgtgcctgtacgccacggtggaacctgacatgtggggcagcgtagaggagctgctccgcgttgtcaaggacaagatcggcgaggagcagcgcaagaccatctgggtggacgaggaccagctg

SEQ ID NO: 20 - protein, 1147 aa, Synthetic construct
MDDYMETLKDEEDALWENVECNRHMLSRYINPAKLTPYLRQCKVIDEQDEDEVLNAPMLPSKINRAGRLLDILHTKGQRGYVVFLESLEFYYPELYKLVTGKEPTRRFSTIVVEEGHEGLTHFLMNEVIKLQQQMKAKDLQRCELLARLRQLEDEKKQMTLTRVELLTFQERYYKMKEERDSYNDELVKVKDDNYNLAMRYAQLSEEKNMAVMRSRDLQLEIDQLKHRLNKMEEECKLERNQSLKLKNDIENRPKKEQVLELERENEMLKTKNQELQSIIQAGKRSLPDSDKAILDILEHDRKEALEDRQELVNRIYNLQEEARQAEELRDKYLEEKEDLELKCSTLGKDCEMYKHRMNTVMLQLEEVERERDQAFHSRDEAQTQYSQCLIEKDKYRKQIRELEEKNDEMRIEMVRREACIVNLESKLRRLSKDSNNLDQSLPRNLPVTIISQDFGDASPRTNGQEADDSSTSEESPEDSKYFLPYHPPQRRMNLKGIQLQRAKSPISLKRTSDFQAKGHEEEGTDASPSSCGSLPITNSFTKMQPPRSRSSIMSITAEPPGNDSIVRRYKEDAPHRSTVEEDNDSGGFDALDLDDDSHERYSFGPSFIHSSSSSHQSEGLDAYDLEQVNLMFRKFSLERPFRPSVTSVGHVRGPGPSVQHTTLNGDSLTSQLTLLGGNARGSFVHSVKPGSLAEKAGLREGHQLLLLEGCIRGERQSVPLDTCTKEEAHWTIQRCSGPVTLHYKVNHEGYRKLVKDMEDGLITSGDSFYIRLNLNISSQLDACTMSLKCDDVVHVRDTMYQDRHEWLCARVDPFTDHDLDMGTIPSYSRAQQLLLVKLQRLMHRGSREEVDGTHHTLRALRNTLQPEEALSTSDPRVSPRLSRASFLFGQLLQFVSRSENKYKRMNSNERVRIISGSPLGSLARSSLDATKLLTEKQEELDPESELGKNLSLIPYSLVRAFYCERRRPVLFTPTVLAKTLVQRLLNSGGAMEFTICKSDIVTRDEFLRRQKTETIIYSREKNPNAFECIAPANIEAVAAKNKHCLLEAGIGCTRDLIKSNIYPIVLFIRVCEKNIKRFRKLLPRPETEEEFLRVCRLKEKELEALPCLYATVEPDMWGSVEELLRVVKDKIGEEQRKTIWVDEDQL

SEQ ID NO: 21 - DNA, 3441 bp, Synthetic construct
  source 1..3441: mol_type = other DNA
atggatgactacatggagacgctgaaggatgaagaggacgccttgtgggagaatgtggagtgtaaccggcacatgctcagccgctatatcaaccctgccaagctcacgccctacctgcgtcagtgtaaggtcattgatgagcaggatgaagatgaagtgcttaatgcccctatgctgccatccaagatcaaccgagcaggccggctgttggacattctacataccaaggggcaaaggggctatgtggtcttcttggagagcctagaattttattacccagaactgtacaaactggtgactgggaaagagcccactcggagattctccaccattgtggtggaggaaggccacgagggcctcacgcacttcctgatgaacgaggtcatcaagctgcagcagcagatgaaggccaaggacctgcaacgctgcgagctgctggccaggttgcggcagctggaggatgagaagaagcagatgacgctgacgcgcgtggagctgctaaccttccaggagcggtactacaagatgaaggaagagcgggacagctacaatgacgagctggtcaaggtgaaggacgacaactacaacttagccatgcgctacgcacagctcagtgaggagaagaacatggcggtcatgaggagccgagacctccaactcgagatcgatcagctaaagcaccggttgaataagatggaggaggaatgtaagctggagagaaatcagtctctaaaactgaagaatgacattgaaaatcggcccaagaaggagcaggttctggaactggagcgggagaatgaaatgctgaagaccaaaaaccaggagctgcagtccatcatccaggccgggaagcgcagcctgccagactcagacaaggccatcctggacatcttggaacacgaccgcaaggaggccctggaggacaggcaggagctggtcaacaggatctacaacctgcaggaggaggcccgccaggcagaggagctgcgagacaagtacctggaggagaaggaggacctggagctcaagtgctcgaccctgggaaaggactgtgaaatgtgcaagcaccgcatgaacacggtcatgctgcagctggaggaggtggagcgggagcgggaccaggccttccactcccgagatgaagctcagacacagtactcgcagtgcttaatcgaaaaggacaagtacaggaagcagatccgcgagctggaggagaagaacgatgagatgaggatcgagatggtgcggcgggaggcctgcatcgtcaacctggagagcaagctgcggcgcctctccaaggacagcaacaacctggaccagagtctgcccaggaacctgccagtaaccatcatctctcaggactttggggatgccagccccaggaccaatggtcaagaagctgacgattcttccacctcggaggagtcacctgaagacagcaagtacttcctgccctaccatccgccccagcgcaggatgaacctgaagggcatccagctgcagagagccaaatcccccatcagcctgaagcgaacatcagattttcaagccaaggggcacgaggaagaaggcacggatgccagccctagctcctgcggatctctgcccatcaccaactccttcaccaagatgcagcccccccggagccgcagcagcatcatgtcaatcaccgccgagcccccgggaaacgactccatcgtcagacgctacaaggaggacgcgccccatcgcagcacagtcgaagaagacaatgacagcggcgggtttgacgccttagatctggatgatgacagtcacgaacgctactccttcggaccctcctccatccactcctcctcctcctcccaccaatccgagggcctggatgcctacgacctggagcaggtcaacctcatgttcaggaagttctctctggaaagacccttccggccttcggtcacctctgtggggcacgtgcggggcccagggccctcggtgcagcacacgacgctgaatggcgacagcctcacctcccagctcaccctgctggggggcaacgcgcgagggagcttcgtgcactcggtcaagcctggctctctggccgagaaagccggcctccgtgagggccaccagctgctgctgctagaaggctgcatccgaggcgagaggcagagtgtcccgttggacacatgcaccaaagaggaagcccactggaccatccagaggtgcagcggccccgtcacgctgcactacaaggtcaaccacgaagggtaccggaagctggtgaaggacatggaggacggcctgatcacatcgggggactcgttctacatccggctgaacctgaacatctccagccagctggacgcctgcaccatgtccctgaagtgtgacgatgttgtgcacgtccgtgacaccatgtaccaggacaggcacgagtggctgtgcgcgcgggtcgaccctttcacagaccatgacctggatatgggcaccatacccagctacagccgagcccagcagctcctcctggtgaaactgcagcgcctgatgcaccgaggcagccgggaggaggtagacggcacccaccacaccctgcgggcactccggaacaccctgcagccagaagaagcgctttcaacaagcgacccccgggtcagcccccgtctctcgcgagcaagcttcctttttggccagctccttcagttcgtcagcaggtccgagaacaagtataagcggatgaacagcaacgagcgggtccgcatcatctcggggagtccgctagggagcctggcccggtcctcgctggacgccaccaagctcttgactgagaagcaggaagagctggaccctgagagcgagctgggcaagaacctcagcctcatcccctacagcctggtacgcgccttctactgcgagcgccgccggcccgtgctcttcacacccaccgtgctggccaagacgctggtgcagaggctgctcaactcgggaggtgccatggagttcaccatctgcaagtcagatatcgtcacaagagatgagttcctcagaaggcagaagacggagaccatcatctactcccgagagaagaaccccaacgcgttcgaatgcatcgcccctgccaacattgaagctgtggccgccaagaacaagcactgcctgctggaggctgggatcggctgcacaagagacttgatcaagtccaacatctaccccatcgtgctcttcatccgggtgtgtgagaagaacatcaagaggttcagaaagctgctgccccgacctgagacggaggaggagttcctgcgcgtgtgccggctgaaggagaaggagctggaggccctgccgtgcctgtacgccacggtggaacctgacatgtggggcagcgtagaggagctgctccgcgttgtcaaggacaagatcggcgaggagcagcgcaagaccatctgggtggacgaggaccagctg

SEQ ID NO: 22 - protein, 1147 aa, Synthetic construct
MDDYMETLKDEEDALWENVECNRHMLSRYINPAKLTPYLRQCKVIDEQDEDEVLNAPMLPSKINRAGRLLDILHTKGQRGYVVFLESLEFYYPELYKLVTGKEPTRRFSTIVVEEGHEGLTHFLMNEVIKLQQQMKAKDLQRCELLARLRQLEDEKKQMTLTRVELLTFQERYYKMKEERDSYNDELVKVKDDNYNLAMRYAQLSEEKNMAVMRSRDLQLEIDQLKHRLNKMEEECKLERNQSLKLKNDIENRPKKEQVLELERENEMLKTKNQELQSIIQAGKRSLPDSDKAILDILEHDRKEALEDRQELVNRIYNLQEEARQAEELRDKYLEEKEDLELKCSTLGKDCEMCKHRMNTVMLQLEEVERERDQAFHSRDEAQTQYSQCLIEKDKYRKQIRELEEKNDEMRIEMVRREACIVNLESKLRRLSKDSNNLDQSLPRNLPVTIISQDFGDASPRTNGQEADDSSTSEESPEDSKYFLPYHPPQRRMNLKGIQLQRAKSPISLKRTSDFQAKGHEEEGTDASPSSCGSLPITNSFTKMQPPRSRSSIMSITAEPPGNDSIVRRYKEDAPHRSTVEEDNDSGGFDALDLDDDSHERYSFGPSSIHSSSSSHQSEGLDAYDLEQVNLMFRKFSLERPFRPSVTSVGHVRGPGPSVQHTTLNGDSLTSQLTLLGGNARGSFVHSVKPGSLAEKAGLREGHQLLLLEGCIRGERQSVPLDTCTKEEAHWTIQRCSGPVTLHYKVNHEGYRKLVKDMEDGLITSGDSFYIRLNLNISSQLDACTMSLKCDDVVHVRDTMYQDRHEWLCARVDPFTDHDLDMGTIPSYSRAQQLLLVKLQRLMHRGSREEVDGTHHTLRALRNTLQPEEALSTSDPRVSPRLSRASFLFGQLLQFVSRSENKYKRMNSNERVRIISGSPLGSLARSSLDATKLLTEKQEELDPESELGKNLSLIPYSLVRAFYCERRRPVLFTPTVLAKTLVQRLLNSGGAMEFTICKSDIVTRDEFLRRQKTETIIYSREKNPNAFECIAPANIEAVAAKNKHCLLEAGIGCTRDLIKSNIYPIVLFIRVCEKNIKRFRKLLPRPETEEEFLRVCRLKEKELEALPCLYATVEPDMWGSVEELLRVVKDKIGEEQRKTIWVDEDQL

SEQ ID NO: 23 - DNA, 3441 bp, Synthetic construct
  source 1..3441: mol_type = other DNA
atggatgactacatggagacgctgaaggatgaagaggacgccttgtgggagaatgtggagtgtaaccggcacatgctcagccgctatatcaaccctgccaagctcacgccctacctgcgtcagtgtaaggtcattgatgagcaggatgaagatgaagtgcttaatgcccctatgctgccatccaagatcaaccgagcaggccggctgttggacattctacataccaaggggcaaaggggctatgtggtcttcttggagagcctagaattttattacccagaactgtacaaactggtgactgggaaagagcccactcggagattctccaccattgtggtggaggaaggccacgagggcctcacgcacttcctgatgaacgaggtcatcaagctgcagcagcagatgaaggccaaggacctgcaacgctgcgagctgctggccaggttgcggcagctggaggatgagaagaagcagatgacgctgacgcgcgtggagctgctaaccttccaggagcggtactacaagatgaaggaagagcgggacagctacaatgacgagctggtcaaggtgaaggacgacaactacaacttagccatgcgctacgcacagctcagtgaggagaagaacatggcggtcatgaggagccgagacctccaactcgagatcgatcagctaaagcaccggttgaataagatggaggaggaatgtaagctggagagaaatcagtctctaaaactgaagaatgacattgaaaatcggcccaagaaggagcaggttctggaactggagcgggagaatgaaatgctgaagaccaaaaaccaggagctgcagtccatcatccaggccgggaagcgcagcctgccagactcagacaaggccatcctggacatcttggaacacgaccgcaaggaggccctggaggacaggcaggagctggtcaacaggatctacaacctgcaggaggaggcccgccaggcagaggagctgcgagacaagtacctggaggagaaggaggacctggagctcaagtgctcgaccctgggaaaggactgtgaaatgtacaagcaccgcatgaacacggtcatgctgcagctggaggaggtggagcgggagcgggaccaggccttccactcccgagatgaagctcagacacagtactcgcagtgcttaatcgaaaaggacaagtacaggaagcagatccgcgagctggaggagaagaacgatgagatgaggatcgagatggtgcggcgggaggcctgcatcgtcaacctggagagcaagctgcggcgcctctccaaggacagcaacaacctggaccagagtctgcccaggaacctgccagtaaccatcatctctcaggactttggggatgccagccccaggaccaatggtcaagaagctgacgattcttccacctcggaggagtcacctgaagacagcaagtacttcctgccctaccatccgccccagcgcaggatgaacctgaagggcatccagctgcagagagccaaatcccccatcagcctgaagcgaacatcagattttcaagccaaggggcacgaggaagaaggcacggatgccagccctagctcctgcggatctctgcccatcaccaactccttcaccaagatgcagcccccccggagccgcagcagcatcatgtcaatcaccgccgagcccccgggaaacgactccatcgtcagacgctacaaggaggacgcgccccatcgcagcacagtcgaagaagacaatgacagcggcgggtttgacgccttagatctggatgatgacagtcacgaacgctactccttcggaccctcctccatccactcctcctcctcctcccaccaatccgagggcctggatgcctacgacctgaagcaggtcaacctcatgttcaggaagttctctctggaaagacccttccggccttcggtcacctgtgtggggcacgtgcggggcccagggccctcggtgcagcacacgacgctgaatggcgacagcctcacctcccagctcaccctgctggggggcaacgcgcgagggagcttcgtgcactcggtcaagcctggctctctggccgagaaagccggcctccgtgagggccaccagctgctgctgctagaaggctgcatccgaggcgagaggcagagtgtcccgttggacacatgcaccaaagaggaagcccactggaccatccagaggtgcagcggccccgtcacgctgcactacaaggtcaaccacgaagggtaccggaagctggtgaaggacatggaggacggcctgatcacatcgggggactcgttctacatccggctgaacctgaacatctccagccagctggacgcctgcaccatgtccctgaagtgtgacgatgttgtgcacgtccgtgacaccatgtaccaggacaggcacgagtggctgtgcgcgcgggtcgaccctttcacagaccatgacctggatatgggcaccatacccagctacagccgagcccagcagctcctcctggtgaaactgcagcgcctgatgcaccgaggcagccgggaggaggtagacggcacccaccacaccctgcgggcactccggaacaccctgcagccagaagaagcgctttcaacaagcgacccccgggtcagcccccgtctctcgcgagcaagcttcctttttggccagctccttcagttcgtcagcaggtccgagaacaagtataagcggatgaacagcaacgagcgggtccgcatcatctcggggagtccgctagggagcctggcccggtcctcgctggacgccaccaagctcttgactgagaagcaggaagagctggaccctgagagcgagctgggcaagaacctcagcctcatcccctacagcctggtacgcgccttctactgcgagcgccgccggcccgtgctcttcacacccaccgtgctggccaagacgctggtgcagaggctgctcaactcgggaggtgccatggagttcaccatctgcaagtcagatatcgtcacaagagatgagttcctcagaaggcagaagacggagaccatcatctactcccgagagaagaaccccaacgcgttcgaatgcatcgcccctgccaacattgaagctgtggccgccaagaacaagcactgcctgctggaggctgggatcggctgcacaagagacttgatcaagtccaacatctaccccatcgtgctcttcatccgggtgtgtgagaagaacatcaagaggttcagaaagctgctgccccgacctgagacggaggaggagttcctgcgcgtgtgccggctgaaggagaaggagctggaggccctgccgtgcctgtacgccacggtggaacctgacatgtggggcagcgtagaggagctgctccgcgttgtcaaggacaagatcggcgaggagcagcgcaagaccatctgggtggacgaggaccagctg

SEQ ID NO: 24 - protein, 1147 aa, Synthetic construct
MDDYMETLKDEEDALWENVECNRHMLSRYINPAKLTPYLRQCKVIDEQDEDEVLNAPMLPSKINRAGRLLDILHTKGQRGYVVFLESLEFYYPELYKLVTGKEPTRRFSTIVVEEGHEGLTHFLMNEVIKLQQQMKAKDLQRCELLARLRQLEDEKKQMTLTRVELLTFQERYYKMKEERDSYNDELVKVKDDNYNLAMRYAQLSEEKNMAVMRSRDLQLEIDQLKHRLNKMEEECKLERNQSLKLKNDIENRPKKEQVLELERENEMLKTKNQELQSIIQAGKRSLPDSDKAILDILEHDRKEALEDRQELVNRIYNLQEEARQAEELRDKYLEEKEDLELKCSTLGKDCEMYKHRMNTVMLQLEEVERERDQAFHSRDEAQTQYSQCLIEKDKYRKQIRELEEKNDEMRIEMVRREACIVNLESKLRRLSKDSNNLDQSLPRNLPVTIISQDFGDASPRTNGQEADDSSTSEESPEDSKYFLPYHPPQRRMNLKGIQLQRAKSPISLKRTSDFQAKGHEEEGTDASPSSCGSLPITNSFTKMQPPRSRSSIMSITAEPPGNDSIVRRYKEDAPHRSTVEEDNDSGGFDALDLDDDSHERYSFGPSSIHSSSSSHQSEGLDAYDLKQVNLMFRKFSLERPFRPSVTCVGHVRGPGPSVQHTTLNGDSLTSQLTLLGGNARGSFVHSVKPGSLAEKAGLREGHQLLLLEGCIRGERQSVPLDTCTKEEAHWTIQRCSGPVTLHYKVNHEGYRKLVKDMEDGLITSGDSFYIRLNLNISSQLDACTMSLKCDDVVHVRDTMYQDRHEWLCARVDPFTDHDLDMGTIPSYSRAQQLLLVKLQRLMHRGSREEVDGTHHTLRALRNTLQPEEALSTSDPRVSPRLSRASFLFGQLLQFVSRSENKYKRMNSNERVRIISGSPLGSLARSSLDATKLLTEKQEELDPESELGKNLSLIPYSLVRAFYCERRRPVLFTPTVLAKTLVQRLLNSGGAMEFTICKSDIVTRDEFLRRQKTETIIYSREKNPNAFECIAPANIEAVAAKNKHCLLEAGIGCTRDLIKSNIYPIVLFIRVCEKNIKRFRKLLPRPETEEEFLRVCRLKEKELEALPCLYATVEPDMWGSVEELLRVVKDKIGEEQRKTIWVDEDQL

SEQ ID NO: 25 - DNA, 3441 bp, Homo sapiens
  source 1..3441: mol_type = genomic DNA
atggatgactacatggagacgctgaaggatgaagaggacgccttgtgggagaatgtggagtgtaaccggcacatgctcagccgctatatcaaccctgccaagctcacgccctacctgcgtcagtgtaaggtcattgatgagcaggatgaagatgaagtgcttaatgcccctatgctgccatccaagatcaaccgagcaggccggctgttggacattctacataccaaggggcaaaggggctatgtggtcttcttggagagcctagaattttattacccagaactgtacaaactggtgactgggaaagagcccactcggagattctccaccattgtggtggaggaaggccacgagggcctcacgcacttcctgatgaacgaggtcatcaagctgcagcagcagatgaaggccaaggacctgcaacgctgcgagctgctggccaggttgcggcagctggaggatgagaagaagcagatgacgctgacgcgcgtggagctgctaaccttccaggagcggtactacaagatgaaggaagagcgggacagctacaatgacgagctggtcaaggtgaaggacgacaactacaacttagccatgcgctacgcacagctcagtgaggagaagaacatggcggtcatgaggagccgagacctccaactcgagatcgatcagctaaagcaccggttgaataagatggaggaggaatgtaagctggagagaaatcagtctctaaaactgaagaatgacattgaaaatcggcccaagaaggagcaggttctggaactggagcgggagaatgaaatgctgaagaccaaaaaccaggagctgcagtccatcatccaggccgggaagcgcagcctgccagactcagacaaggccatcctggacatcttggaacacgaccgcaaggaggccctggaggacaggcaggagctggtcaacaggatctacaacctgcaggaggaggcccgccaggcagaggagctgcgagacaagtacctggaggagaaggaggacctggagctcaagtgctcgaccctgggaaaggactgtgaaatgtacaagcaccgcatgaacacggtcatgctgcagctggaggaggtggagcgggagcgggaccaggccttccactcccgagatgaagctcagacacagtactcgcagtgcttaatcgaaaaggacaagtacaggaagcagatccgcgagctggaggagaagaacgatgagatgaggatcgagatggtgcggcgggaggcctgcatcgtcaacctggagagcaagctgcggcgcctctccaaggacagcaacaacctggaccagagtctgcccaggaacctgccagtaaccatcatctctcaggactttggggatgccagccccaggaccaatggtcaagaagctgacgattcttccacctcggaggagtcacctgaagacagcaagtacttcctgccctaccatccgccccagcgcaggatgaacctgaagggcatccagctgcagagagccaaatcccccatcagcctgaagcgaacatcagattttcaagccaaggggcacgaggaagaaggcacggatgccagccctagctcctgcggatctctgcccatcaccaactccttcaccaagatgcagcccccccggagccgcagcagcatcatgtcaatcaccgccgagcccccgggaaacgactccatcgtcagacgctacaaggaggacgcgccccatcgcagcacagtcgaagaagacaatgacagcggcgggtttgacgccttagatctggatgatgacagtcacgaacgctactccttcggaccctcctccatccactcctcctcctcctcccaccaatccgagggcctggatgcctacgacctggagcaggtcaacctcatgttcaggaagttctctctggaaagacccttccggccttcggtcacctctgtggggcacgtgcggggcccagggccctcggtgcagcacacgacgctgaatggcgacagcctcacctcccagctcaccctgctggggggcaacgcgcgagggagcttcgtgcactcggtcaagcctggctctctggccgagaaagccggcctccgtgagggccaccagctgctgctgctagaaggctgcatccgaggcgagaggcagagtgtcccgttggacacatgcaccaaagaggaagcccactggaccatccagaggtgcagcggccccgtcacgctgcactacaaggtcaaccacgaagggtaccggaagctggtgaaggacatggaggacggcctgatcacatcgggggactcgttctacatccggctgaacctgaacatctccagccagctggacgcctgcaccatgtccctgaagtgtgacgatgttgtgcacgtccgtgacaccatgtaccaggacaggcacgagtggctgtgcgcgcgggtcgaccctttcacagaccatgacctggatatgggcaccatacccagctacagccgagcccagcagctcctcctggtgaaactgcagcgcctgatgcaccgaggcagccgggaggaggtagacggcacccaccacaccctgcgggcactccggaacaccctgcagccagaagaagcgctttcaacaagcgacccccgggtcagcccccgtctctcgcgagcaagcttcctttttggccagctccttcagttcgtcagcaggtccgagaacaagtataagcggatgaacagcaacgagcgggtccgcatcatctcggggagtccgctagggagcctggcccggtcctcgctggacgccaccaagctcttgactgagaagcaggaagagctggaccctgagagcgagctgggcaagaacctcagcctcatcccctacagcctggtacgcgccttctactgcgagcgccgccggcccgtgctcttcacacccaccgtgctggccaagacgctggtgcagaggctgctcaactcgggaggtgccatggagttcaccatctgcaagtcagatatcgtcacaagagatgagttcctcagaaggcagaagacggagaccatcatctactcccgagagaagaaccccaacgcgttcgaatgcatcgcccctgccaacattgaagctgtggccgccaagaacaagcactgcctgctggaggctgggatcggctgcacaagagacttgatcaagtccaacatctaccccatcgtgctcttcatccgggtgtgtgagaagaacatcaagaggttcagaaagctgctgccccgacctgagacggaggaggagttcctgcgcgtgtgccggctgaaggagaaggagctggaggccctgccgtgcctgtacgccacggtggaacctgacatgtggggcagcgtagaggagctgctccgcgttgtcaaggacaagatcggcgaggagcagcgcaagaccatctgggtggacgaggaccagctg

SEQ ID NO: 26 - protein, 1154 aa, Homo sapiens [sequence omitted]

SEQ ID NO: 27 - DNA, 4305 bp, Homo sapiens
  source 1..4305: mol_type = genomic DNA
atggcccagacccccgacggcatctcctgtgagctccgaggcgagatcaccaggttcctgtggcccaaagaggtggagctgctgctgaaaacctggctacccggggagggtgctgtgcaaaaccatgtcctggcactgctacgatggagagcctacctgctgcacaccacctgcctcccgctgagggtggactgcacgttcagctacctggaggtccaggccatggcgctgcaggagacaccccctcaggtcacctttgagctggagtccctgcgtgagctggtcctggagtttcctggtgtggccgccctggaacagctggcccagcacgtggctgcagccatcaagaaggtcttccctcgctcgacccttgggaagctattccggaggcccacaccagcctccatgctggctcggctggagagaagcagcccctcggagtccactgacccctgcagcccctgtggtggcttcttggagacatacgaggctctgtgtgactacaatggcttccctttccgagaggagattcagtgggacgtggacaccatttaccatcgccagggctgccgccatttcagcctgggagacttcagccacctcggcagtcgggacctggccttgagtgtggctgccctgtcctacaacctgtggttccggtgcctctcctgtgtggacatgaagctgagccttgaggtctcagaacagattctgcacatgatgagtcagtcatcacacctggaggagctggtgctggagacctgcagcctgaggggagactttgtccgacgactggcccaggcgctggcgggacactcaagctctgggctgcgggagctcagcctcgcggggaacctgctggatgaccgaggcatgactgcactcagcagacacctcgagcgttgtccaggagccctgaggagactcagcctggcccagacagggttgacaccgcgaggaatgagggctctgggccgggcactggccaccaatgccgccttcgactccaccctgacccacctggacctttctgggaatcctggggcgctgggggcctccgaggacagtgggggcctctatagcttcctgagccgtcctaacgtactgtcgttcctgaatctcgcaggcaccgacactgccctggacactgtgagggggtgctccgtggggggatggatgaccggcagggcggactggagggcgggacggggagggctcggtccccccgcgggtgtagccaacagcctccccccgcagctcttcgcagcggtatcccgaggctgctgcaccagccttacccacctcgacgcttcgaggaacgtcttctcccgcacgaagtcccgcgccgcgccggccgcgctgcagctcttcctcagccgcgcgcggacgctgcggcacctgggcctggcgggctgcaagctgccgcccgacgcgctcagagcccttttggatggcctcgcgctcaacacgcacctccgcgacctgcacctggacctcagcgcttgcgagctgcgctcggccggcgcccaggtgatacaagacttagtgtgcgacgcaggcgctgtgagctccctggatctggcggataacggcttcggctcagacatggtgactctggtgctggccatcgggagaagccggtccctgagacatgtggcgcttggaaggaacttcaacgtccggtgcaaggagaccctggacgacgtcctgcaccggattgtccagctcatgcaggacgacgattgtcctcttcagtctctgtcggtggctgagtctcggctgaagctgggtgccagcgtcctactccgggccctagccaccaatcctaacctgaccgcgctggatatcagcggcaacgccatgggggacgcgggcgccaagttgctggccaaggcgctgcgggtcaactcgaggctccggtctgtggtctgggaccggaaccacacatctgctttgggtctgctggacgtggcgcaggcgctggagcagaaccacagcctgaaggccatgcctctgccactgaacgacgtggcccaggcgcagcgcagccgcccggaactgacagcacgtgcagtccatcagatccaagcctgtctcttgaggaacaaccgcgcagaccctgcctcttctgaccacacgacccgccttcagccacttggtctggtctcagacccctcagagcaggaagtgaatgaattgtgtcagtcggtgcaggagcatgtggagctgctgggctgtggggctgggccccagggtgaagccgctgtgcgccaggccgaggatgccatccaaaatgccaacttctctctcagcattctccccattctatatgaagctggaagctccccaagccatcactggcagcttgggcagaagctggagggccttctgagacaggtgggcgaggtctgccgccaggacatccaggacttcactcaggccacactggacacagcaaggagcctctgcccacagatgctgcagggatccagctggagggagcagctagagggggtcctggcaggctcgaggggcctcccggagctgctcccagagcagctgctgcaagatgccttcactaggctcagggacatgcggctatcaatcacggggaccttggcagagagcattgtggctcaggctttggcaggcctgagtgcagcccgggatcagctggtggagagtctggctcagcaggcaacagtgacaatgccccctgccctaccagcaccggatggaggtgagcccagcctccttgagcctggggaattggaaggtcttttcttccccgaggagaaggaagaggagaaggagaaggatgacagtcctccacagaaatggcctgagctcagccacggtcttcacctggtccccttcattcacagtgctgctgaggaagcggagccggagcccgagctggcggctccgggagaagatgcagagccgcaggcggggccgtccgcgcgcggctctccgagccctgccgcccctgggcccccggccggcccgctgccccgcatggacctgccactggcggggcagcccctgcgccatccgacccgggcccggccgcggccgcgccgccagcaccaccaccgcccgccgccggggggcccccaggtacccccagccttgccgcaggaagggaatgggctcagtgcccgcgtggacgagggcgtggaggaattcttctccaaaaggctgatccagcaggatcgcctctgggcccccgaggaggacccggccactgaggggggcgccactcctgtcccccgtacactgcgaaagaagctgggcaccctctttgccttcaagaagcctcgttcaacgcggggtccacggactgatctagagaccagccctggggcagctccccgaacccgaaaaactacatttggcgacctactgcggccgccaacccgtcccagccgtggtgaggagcttggtggggctgagggggacaccagcagccctgaccctgccggcaggagccgacctcgctacacaagagatagcaaggcctactcgatgatactgctgcctgccgaggaggaggcaacgctgggtgccagacccgacaagcggcggcccctggagcggggagaaacagaactggctccatcctttgaacagcgggtacaagtaatgctgcagaggataggcgtcagccgaggcagcgggggtgccgaaggcaagaggaagcaaagcaaagatggcgagatcaagaaagctggctcagatggtgacattatggacagttccacggaggcccctcccatctcgatcaagtcccgcacccactctgtgtctgctgacccttcctgcagacctggcccagggagccaggggcctgagtctgccacctggaagacactggggcagcagttgaatgcggagctcaggagccgtggttggggccaacaggatggtccaggccctccctcccctggtcaaagcccaagtccctgcagaaccagcccctccccagacagcctgggcctcccagaggacccttgcttgggccccagaaatgaagatggccagctgaggccgaggcctctctcggcagggcggcgagcagtgtctgtgcatgaggaccagctccaggcccctgctgaacggcccctgaggctgcagcgctcccccgtcctcaaacgcaggccaaaactcgaggcacctccatccccaagcctaggatctggccttggaaccgagcctctgcccccacagcccacagagccctccagccctgagcggagcccaccctccccagccacagaccaaagaggcggcggccccaatccc

SEQ ID NO: 28 - protein, 1435 aa, Homo sapiens
MAQTPDGISCELRGEITRFLWPKEVELLLKTWLPGEGAVQNHVLALLRWRAYLLHTTCLPLRVDCTFSYLEVQAMALQETPPQVTFELESLRELVLEFPGVAALEQLAQHVAAAIKKVFPRSTLGKLFRRPTPASMLARLERSSPSESTDPCSPCGGFLETYEALCDYNGFPFREEIQWDVDTIYHRQGCRHFSLGDFSHLGSRDLALSVAALSYNLWFRCLSCVDMKLSLEVSEQILHMMSQSSHLEELVLETCSLRGDFVRRLAQALAGHSSSGLRELSLAGNLLDDRGMTALSRHLERCPGALRRLSLAQTGLTPRGMRALGRALATNAAFDSTLTHLDLSGNPGALGASEDSGGLYSFLSRPNVLSFLNLAGTDTALDTVRGCSVGGWMTGRADWRAGRGGLGPPAGVANSLPPQLFAAVSRGCCTSLTHLDASRNVFSRTKSRAAPAALQLFLSRARTLRHLGLAGCKLPPDALRALLDGLALNTHLRDLHLDLSACELRSAGAQVIQDLVCDAGAVSSLDLADNGFGSDMVTLVLAIGRSRSLRHVALGRNFNVRCKETLDDVLHRIVQLMQDDDCPLQSLSVAESRLKLGASVLLRALATNPNLTALDISGNAMGDAGAKLLAKALRVNSRLRSVVWDRNHTSALGLLDVAQALEQNHSLKAMPLPLNDVAQAQRSRPELTARAVHQIQACLLRNNRADPASSDHTTRLQPLGLVSDPSEQEVNELCQSVQEHVELLGCGAGPQGEAAVRQAEDAIQNANFSLSILPILYEAGSSPSHHWQLGQKLEGLLRQVGEVCRQDIQDFTQATLDTARSLCPQMLQGSSWREQLEGVLAGSRGLPELLPEQLLQDAFTRLRDMRLSITGTLAESIVAQALAGLSAARDQLVESLAQQATVTMPPALPAPDGGEPSLLEPGELEGLFFPEEKEEEKEKDDSPPQKWPELSHGLHLVPFIHSAAEEAEPEPELAAPGEDAEPQAGPSARGSPSPAAPGPPAGPLPRMDLPLAGQPLRHPTRARPRPRRQHHHRPPPGGPQVPPALPQEGNGLSARVDEGVEEFFSKRLIQQDRLWAPEEDPATEGGATPVPRTLRKKLGTLFAFKKPRSTRGPRTDLETSPGAAPRTRKTTFGDLLRPPTRPSRGEELGGAEGDTSSPDPAGRSRPRYTRDSKAYSMILLPAEEEATLGARPDKRRPLERGETELAPSFEQRVQVMLQRIGVSRGSGGAEGKRKQSKDGEIKKAGSDGDIMDSSTEAPPISIKSRTHSVSADPSCRPGPGSQGPESATWKTLGQQLNAELRSRGWGQQDGPGPPSPGQSPSPCRTSPSPDSLGLPEDPCLGPRNEDGQLRPRPLSAGRRAVSVHEDQLQAPAERPLRLQRSPVLKRRPKLEAPPSPSLGSGLGTEPLPPQPTEPSSPERSPPSPATDQRGGGPNP

SEQ ID NO: 29 - DNA, 4305 bp, Synthetic construct
  source 1..4305: mol_type = other DNA
atggcccagacccccgacggcatctcctgtgagctccgaggcgagatcaccaggttcctgtggcccaaagaggtggagctgctgctgaaaacctggctacccggggagggtgctgtgcaaaaccatgtcctggcactgctacgatggagagcctacctgctgcacaccacctgcctcccgctgagggtggactgcacgttcagctacctggaggtccaggccatggcgctgcaggagacaccccctcaggtcacctttgagctggagtccctgcgtgagctggtcctggagtttcctggtgtggccgccctggaacagctggcccagcacgtggctgcagccatcaagaaggtcttccctcgctcgacccttgggaagctattccggaggcccacaccagcctccatgctggctcggctggagagaagcagcccctcggagtccactgacccctgcagcccctgtggtggcttcttggagacatacgaggctctgtgtgactacaatggcttccctttccgagaggagattcagtgggacgtggacaccatttaccatcgccagggctgccgccatttcagcctgggagacttcagccacctcggcagtcgggacctggccttgagtgtggctgccctgtcctacaacctgtggttccggtgcctctcctgtgtggacatgaagctgagccttgaggtctcagaacagattctgcacatgatgagtcagtcatcacacctggaggagctggtgctggagacctgcagcctgaggggagactttgtccgacgactggcccaggcgctggcgggacactcaagctctgggctgcgggagctcagcctcgcggggaacctgctggatgaccgaggcatgactgcactcagcagacacctcgagcgttgtccaggagccctgaggagactcagcctggcccagacagggttgacaccgcgaggaatgagggctctgggccgggcactggccaccaatgccgccttcgactccaccctgacccacctggacctttctgggaatcctggggcgctgggggcctccgaggacagtgggggcctctatagcttcctgagccgtcctaacgtactgtcgttcctgaatctcgcaggcaccgacactgccctggacactgtgagggggtgctccgtggggggatggatgaccggcagggcggactggagggcgggacggggagggctcggtccccccgcgggtgtagccaacagcctccccccgcagctcttcgcagcggtatcccgaggctgctgcaccagccttacccacctcgacgcttcgaggaacgtcttctcccgcacgaagtcccgcgccgcgccggccgcgctgcagctcttcctcagccgcgcgcggacgctgcggcacctgggcctggcgggctgcaagctgccgcccgacgcgctcagagcccttttggatggcctcgcgctcaacacgcacctccgcgacctgcacctggacctcagcgcttgcgagctgcgctcggccggcgcccaggtgatacaagacttagtgtgcgacgcaggcgctgtgagctccctggatctggcggataacggcttcggctcagacatggtgactctggtgctggccatcgggagaagccggtccctgagacatgtggcgcttggaaggaacttcaacgtccggtgcaaggagaccctggacgacgtcctgcaccggattgtcgagctcatgcaggacgacgattgtcctcttcagtctctgtcggtggctgagtctcggctgaagctgggtgccagcgtcctactccgggccctagccaccaatcctaacctgaccgcgctggatatcagcggcaacgccatgggggacgcgggcgccaagttgctggccaaggcgctgcgggtcaactcgaggctccggtctgtggtctgggaccggaaccacacatctgctttgggtctgctggacgtggcgcaggcgctggagcagaaccacagcctgaaggccatgcctctgccactgaacgacgtggcccaggcgcagcgcagccgcccggaactgacagcacgtgcagtccatcagatccaagcctgtctcttgaggaacaaccgcgcagaccctgcctcttctgaccacacgacccgccttcagccacttggtctggtctcagacccctcagagcaggaagtgaatgaattgtgtcagtcggtgcaggagcatgtggagctgctgggctgtggggctgggccccagggtgaagccgctgtgcgccaggccgaggatgccatccaaaatgccaacttctctctcagcattctccccattctatatgaagctggaagctccccaagccatcactggcagcttgggcagaagctggagggccttctgagacaggtgggcgaggtctgccgccaggacatccaggacttcactcaggccacactggacacagcaaggagcctctgcccacagatgctgcagggatccagctggagggagcagctagagggggtcctggcaggctcgaggggcctcccggagctgctcccagagcagctgctgcaagatgccttcactaggctcagggacatgcggctatcaatcacggggaccttggcagagagcattgtggctcaggctttggcaggcctgagtgcagcccgggatcagctggtggagagtctggctcagcaggcaacagtgacaatgccccctgccctaccagcaccggatggaggtgagcccagcctccttgagcctggggaattggaaggtcttttcttccccgaggagaaggaagaggagaaggagaaggatgacagtcctccacagaaatggcctgagctcagccacggtcttcacctggtccccttcattcacagtgctgctgaggaagcggagccggagcccgagctggcggctccgggagaagatgcagagccgcaggcggggccgtccgcgcgcggctctccgagccctgccgcccctgggcccccggccggcccgctgccccgcatggacctgccactggcggggcagcccctgcgccatccgacccgggcccggccgcggccgcgccgccagcaccaccaccgcccgccgccggggggcccccaggtacccccagccttgccgcaggaagggaatgggctcagtgcccgcgtggacgagggcgtggaggaattcttctccaaaaggctgatccagcaggatcgcctctgggcccccgaggaggacccggccactgaggggggcgccactcctgtcccccgtacactgcgaaagaagctgggcaccctctttgccttcaagaagcctcgttcaacgcggggtccacggactgatctagagaccagccctggggcagctccccgaacccgaaaaactacatttggcgacctactgcggccgccaacccgtcccagccgtggtgaggagcttggtggggctgagggggacaccagcagccctgaccctgccggcaggagccgacctcgctacacaagagatagcaaggcctactcgatgatactgctgcctgccgaggaggaggcaacgctgggtgccagacccgacaagcggcggcccctggagcggggagaaacagaactggctccatcctttgaacagcgggtacaagtaatgctgcagaggataggcgtcagccgaggcagcgggggtgccgaaggcaagaggaagcaaagcaaagatggcgagatcaagaaagctggctcagatggtgacattatggacagttccacggaggcccctcccatctcgatcaagtcccgcacccactctgtgtctgctgacccttcctgcagacctggcccagggagccaggggcctgagtctgccacctggaagacactggggcagcagttgaatgcggagctcaggagccgtggttggggccaacaggatggtccaggccctccctcccctggtcaaagcccaagtccctgcagaaccagcccctccccagacagcctgggcctcccagaggacccttgcttgggccccagaaatgaagatggccagctgaggccgaggcctctctcggcagggcggcgagcagtgtctgtgcatgaggaccagctccaggcccctgctgaacggcccctgaggctgcagcgctcccccgtcctcaaacgcaggccaaaactcgaggcacctccatccccaagcctaggatctggccttggaaccgagcctctgcccccacagcccacagagccctccagccctgagcggagcccaccctccccagccacagaccaaagaggcggcggccccaatccc

SEQ ID NO: 30 - protein, 1435 aa, Synthetic construct
MAQTPDGISCELRGEITRFLWPKEVELLLKTWLPGEGAVQNHVLALLRWRAYLLHTTCLPLRVDCTFSYLEVQAMALQETPPQVTFELESLRELVLEFPGVAALEQLAQHVAAAIKKVFPRSTLGKLFRRPTPASMLARLERSSPSESTDPCSPCGGFLETYEALCDYNGFPFREEIQWDVDTIYHRQGCRHFSLGDFSHLGSRDLALSVAALSYNLWFRCLSCVDMKLSLEVSEQILHMMSQSSHLEELVLETCSLRGDFVRRLAQALAGHSSSGLRELSLAGNLLDDRGMTALSRHLERCPGALRRLSLAQTGLTPRGMRALGRALATNAAFDSTLTHLDLSGNPGALGASEDSGGLYSFLSRPNVLSFLNLAGTDTALDTVRGCSVGGWMTGRADWRAGRGGLGPPAGVANSLPPQLFAAVSRGCCTSLTHLDASRNVFSRTKSRAAPAALQLFLSRARTLRHLGLAGCKLPPDALRALLDGLALNTHLRDLHLDLSACELRSAGAQVIQDLVCDAGAVSSLDLADNGFGSDMVTLVLAIGRSRSLRHVALGRNFNVRCKETLDDVLHRIVELMQDDDCPLQSLSVAESRLKLGASVLLRALATNPNLTALDISGNAMGDAGAKLLAKALRVNSRLRSVVWDRNHTSALGLLDVAQALEQNHSLKAMPLPLNDVAQAQRSRPELTARAVHQIQACLLRNNRADPASSDHTTRLQPLGLVSDPSEQEVNELCQSVQEHVELLGCGAGPQGEAAVRQAEDAIQNANFSLSILPILYEAGSSPSHHWQLGQKLEGLLRQVGEVCRQDIQDFTQATLDTARSLCPQMLQGSSWREQLEGVLAGSRGLPELLPEQLLQDAFTRLRDMRLSITGTLAESIVAQALAGLSAARDQLVESLAQQATVTMPPALPAPDGGEPSLLEPGELEGLFFPEEKEEEKEKDDSPPQKWPELSHGLHLVPFIHSAAEEAEPEPELAAPGEDAEPQAGPSARGSPSPAAPGPPAGPLPRMDLPLAGQPLRHPTRARPRPRRQHHHRPPPGGPQVPPALPQEGNGLSARVDEGVEEFFSKRLIQQDRLWAPEEDPATEGGATPVPRTLRKKLGTLFAFKKPRSTRGPRTDLETSPGAAPRTRKTTFGDLLRPPTRPSRGEELGGAEGDTSSPDPAGRSRPRYTRDSKAYSMILLPAEEEATLGARPDKRRPLERGETELAPSFEQRVQVMLQRIGVSRGSGGAEGKRKQSKDGEIKKAGSDGDIMDSSTEAPPISIKSRTHSVSADPSCRPGPGSQGPESATWKTLGQQLNAELRSRGWGQQDGPGPPSPGQSPSPCRTSPSPDSLGLPEDPCLGPRNEDGQLRPRPLSAGRRAVSVHEDQLQAPAERPLRLQRSPVLKRRPKLEAPPSPSLGSGLGTEPLPPQPTEPSSPERSPPSPATDQRGGGPNP

SEQ ID NO: 31 - DNA, 879 bp, Homo sapiens
  source 1..879: mol_type = genomic DNA
atggagctgctgtgttgcgaaggcacccggcacgcgccccgggccgggccggacccgcggctgctgggggaccagcgtgtcctgcagagcctgctccgcctggaggagcgctacgtaccccgcgcctcctacttccagtgcgtgcagcgggagatcaagccgcacatgcggaagatgctggcttactggatgctggaggtatgtgaggagcagcgctgtgaggaggaagtcttccccctggccatgaactacctggatcgctacctgtcttgcgtccccacccgaaaggcgcagttgcagctcctgggtgcggtctgcatgctgctggcctccaagctgcgcgagaccacgcccctgaccatcgaaaaactgtgcatctacaccgaccacgctgtctctccccgccagttgcgggactgggaggtgctggtcctagggaagctcaagtgggacctggctgctgtgattgcacatgatttcctggccttcattctgcaccggctctctctgccccgtgaccgacaggccttggtcaaaaagcatgcccagacctttttggccctctgtgctacagattatacctttgccatgtacccgccatccatgatcgccacgggcagcattggggctgcagtgcaaggcctgggtgcctgctccatgtccggggatgagctcacagagctgctggcagggatcactggcactgaagtggactgcctgcgggcctgtcaggagcagatcgaagctgcactcagggagagcctcagggaagcctctcagaccagctccagcccagcgcccaaagccccccggggctccagcagccaagggcccagccagaccagcactcctacagatgtcacagccatacacctgttg

SEQ ID NO: 32 - protein, 293 aa, Homo sapiens
MELLCCEGTRHAPRAGPDPRLLGDQRVLQSLLRLEERYVPRASYFQCVQREIKPHMRKMLAYWMLEVCEEQRCEEEVFPLAMNYLDRYLSCVPTRKAQLQLLGAVCMLLASKLRETTPLTIEKLCIYTDHAVSPRQLRDWEVLVLGKLKWDLAAVIAHDFLAFILHRLSLPRDRQALVKKHAQTFLALCATDYTFAMYPPSMIATGSIGAAVQGLGACSMSGDELTELLAGITGTEVDCLRACQEQIEAALRESLREASQTSSSPAPKAPRGSSSQGPSQTSTPTDVTAIHLL

SEQ ID NO: 33 - DNA, 879 bp, Synthetic construct
  source 1..879: mol_type = other DNA
atggagctgctgtgttgcgaaggcacccggcacgcgccccgggccgggccggacccgcggctgctgggggaccagcgtgtcctgcagagcctgctccgcctggaggagcgctacgtaccccgcgcctcctacttccagtgcgtgcagcgggagatcaagccgcacatgcggaagatgctggcttactggatgctggaggtatgtgaggagcagcgctgtgaggaggaagtcttccccctggccatgaactacctggatcgctacctgtcttgcgtccccacccgaaaggcgcagttgcagctcctgggtgcggtctgcatgctgctggcctccaagctgcgcgagaccacgcccctgaccatcgaaaaactgtgcatctacaccgaccacgctgtctctccccgccagttgcgggactgggaggtgctggtcctagggaagctcaagtgggacctggctgctgtgattgcacatgatttcctggccttcattctgcaccggctctctctgccccgtgaccgacaggccttggtcaaaaagcatgcccagacctttttggccctctgtgctacagattatacctttgccatgtacccgccatccatgatcgccacgggcagcattggggctgcagtgcaaggcctgggtgcctgctccatgtccggggatgagctcacagagctgctggcagggatcactggcactgaagtggactgcctgcgggcctgtcaggagcagatcgaagctgcactcagggagagcctcagggaagcctctcagaccagctccagcccagcgcccaaagccccccggggctccagcagccaagggcccagccagaccagcacttctacagatgtcacagccatacacctgttg

SEQ ID NO: 34 - protein, 293 aa, Synthetic construct
MELLCCEGTRHAPRAGPDPRLLGDQRVLQSLLRLEERYVPRASYFQCVQREIKPHMRKMLAYWMLEVCEEQRCEEEVFPLAMNYLDRYLSCVPTRKAQLQLLGAVCMLLASKLRETTPLTIEKLCIYTDHAVSPRQLRDWEVLVLGKLKWDLAAVIAHDFLAFILHRLSLPRDRQALVKKHAQTFLALCATDYTFAMYPPSMIATGSIGAAVQGLGACSMSGDELTELLAGITGTEVDCLRACQEQIEAALRESLREASQTSSSPAPKAPRGSSSQGPSQTSTSTDVTAIHLL

SEQ ID NO: 35 - DNA, 660 bp, Synthetic construct
  source 1..660: mol_type = other DNA
atgctcaggctgctcttggctctcaacttattcccttcaattcaagtaacaggaaacaagattttggtgaagcagtcgcccatgcttgtagcgtacgacaatgcggtcaaccttagctgcaagtattcctacaatctcttctcaagggaggtccgggcatcccttcacaaaggactggatagtgctgtggaagtctgtgttgtatatgggaattactcccagcagcttcaggtttactcaaaaacggggttcaactgtgatgggaaattgggcaatgaatcagtgacattctacctccagaatttgtatgttaaccaaacagatatttacttctgcaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 36 - protein, 220 aa, Synthetic construct
MLRLLLALNLFPSIQVTGNKILVKQSPMLVAYDNAVNLSCKYSYNLFSREVRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 37 - DNA, 660 bp, Synthetic construct
  source 1..660: mol_type = other DNA
atgctcaggctgctcttggctctcaacttattcccttcaattcaagtaacaggaaacaagattttggtgaagcagtcgcccatgcttgtagcgtacgacaatgcggtcaaccttagctgcaagtattcctacaatctcttctcaagggagatccgggcatcccttcacaaaggactggatagtgctgtggaagtctgtgttgtatatgggaattactcccagcagcttcaggtttactcaaaaacggggttcaactgtgatgggaaattgggcaatgaatcagtgacattctacctccagaatttgtatgttaaccaaacagatatttacttctgcaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 38 - protein, 220 aa, Synthetic construct
MLRLLLALNLFPSIQVTGNKILVKQSPMLVAYDNAVNLSCKYSYNLFSREIRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 39 - DNA, 660 bp, Synthetic construct
  source 1..660: mol_type = other DNA
atgctcaggctgctcttggctctcaacttattcccttcaattcaagtaacaggaaacaagattttggtgaagcagtcgcccatgcttgtagcgtacgacaatgcggtcaaccttagctgcaagtattcctacaatctcttctcaagggagttccgggcatcccttcacaaaggactggatagtgctgtggaagtctgtgttgtatatgggaattactcccagcagcttccggtttactcaaaaacggggttcaactgtgatgggaaattgggcaatgaatcagtgacattctacctccagaatttgtatgttaaccaaacagatatttacttctgcaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 40 - protein, 220 aa, Synthetic construct
MLRLLLALNLFPSIQVTGNKILVKQSPMLVAYDNAVNLSCKYSYNLFSREFRASLHKGLDSAVEVCVVYGNYSQQLPVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 41 - DNA, 660 bp, Homo sapiens
  source 1..660: mol_type = genomic DNA
atgctcaggctgctcttggctctcaacttattcccttcaattcaagtaacaggaaacaagattttggtgaagcagtcgcccatgcttgtagcgtacgacaatgcggtcaaccttagctgcaagtattcctacaatctcttctcaagggagttccgggcatcccttcacaaaggactggatagtgctgtggaagtctgtgttgtatatgggaattactcccagcagcttcaggtttactcaaaaacggggttcaactgtgatgggaaattgggcaatgaatcagtgacattctacctccagaatttgtatgttaaccaaacagatatttacttctgcaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 42 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 43 - DNA, 660 bp, Synthetic construct
  source 1..660: mol_type = other DNA
atgctcaggctgctcttggctctcaacttattcccttcaattcaagtaacaggaaacaagattttggtgaagcagtcgcccatgcttgtagcgtacgacaatgcggtcaaccttagctgcaagtattcctacaatctcttctcaagggagttccgggcatcccttcacaaaggactggatagtgctgtggaagtctgtgttgtatatgggaattactcccagcagcttcaggtttactcaaaaacggggttcaactgtgatgggaaattgggcaatgaatcagtgacattctacctccagaatttgtatgttaaccaaacagatatttacttctgcaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgcctccccgccgccccgggcccacccgcaagcattaccagccctatgccccaccacgcgacttcgcagcctatcgctcc

SEQ ID NO: 44 - protein, 220 aa, Synthetic construct
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMPPRRPGPTRKHYQPYAPPRDFAAYRS

SEQ ID NO: 45 - DNA, 621 bp, Homo sapiens
  source 1..621: mol_type = genomic DNA
atgcagtcgggcactcactggagagttctgggcctctgcctcttatcagttggcgtttgggggcaagatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtagccacaattgtcatagtggacatctgcatcactgggggcttgctgctgctggtttactactggagcaagaatagaaaggccaaggccaagcctgtgacacgaggagcaggtgctggcggcaggcaaaggggacaaaacaaggagaggccaccacctgttcccaacccagactatgagcccatccggaaaggccagcgggacctgtattctggcctgaatcagagacgcatc

SEQ ID NO: 46 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 47 - DNA, 621 bp, Synthetic construct
  source 1..621: mol_type = other DNA
atgcagtcgggcactcactggagagttctgggcctctgcctcttatcagttggcgtttgggggcaagatggtaatgaagaaatgggtggtattacacagacaccatataaagtctccatctgcggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtagccacaattgtcatagtggacatctgcatcactgggggcttgctgctgctggtttactactggagcaagaatagaaaggccaaggccaagcctgtgacacgaggagcaggtgctggcggcaggcaaaggggacaaaacaaggagaggccaccacctgttcccaacccagactatgagcccatccggaaaggccagcgggacctgtattctggcctgaatcagagacgcatc

SEQ ID NO: 48 - protein, 207 aa, Synthetic construct
MQSGTHWRVLGLCLLSVGVWGQDGNEEMGGITQTPYKVSICGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 49 - DNA, 1011 bp, Homo sapiens
  source 1..1011: mol_type = genomic DNA
atggcgagtagcagcggctccaaggctgaattcattgtcggagggaaatataaactggtacggaagatcgggtctggctccttcggggacatctatttggcgatcaacatcaccaacggcgaggaagtggcagtgaagctagaatctcagaaggccaggcatccccagttgctgtacgagagcaagctctataagattcttcaaggtggggttggcatcccccacatacggtggtatggtcaggaaaaagactacaatgtactagtcatggatcttctgggacctagcctcgaagacctcttcaatttctgttcaagaaggttcacaatgaaaactgtacttatgttagctgaccagatgatcagtagaattgaatatgtgcatacaaagaattttatacacagagacattaaaccagataacttcctaatgggtattgggcgtcactgtaataagttattccttattgattttggtttggccaaaaagtacagagacaacaggacaaggcaacacataccatacagagaagataaaaacctcactggcactgcccgatatgctagcatcaatgcacatcttggtattgagcagagtcgccgagatgacatggaatcattaggatatgttttgatgtattttaatagaaccagcctgccatggcaagggctaaaggctgcaacaaagaaaaaaaaatatgaaaagattagtgaaaagaagatgtccacgcctgttgaagttttatgtaaggggtttcctgcagaatttgcgatgtacttaaactattgtcgtgggctacgctttgaggaagccccagattacatgtatctgaggcagctattccgcattcttttcaggaccctgaaccatcaatatgactacacatttgattggacaatgttaaagcagaaagcagcacagcaggcagcctcttccagtgggcagggtcagcaggcccaaacccccacaggcaagcaaactgacaaaaccaagagtaacatgaaaggtttc

SEQ ID NO: 50 - protein, 337 aa, Homo sapiens
MASSSGSKAEFIVGGKYKLVRKIGSGSFGDIYLAINITNGEEVAVKLESQKARHPQLLYESKLYKILQGGVGIPHIRWYGQEKDYNVLVMDLLGPSLEDLFNFCSRRFTMKTVLMLADQMISRIEYVHTKNFIHRDIKPDNFLMGIGRHCNKLFLIDFGLAKKYRDNRTRQHIPYREDKNLTGTARYASINAHLGIEQSRRDDMESLGYVLMYFNRTSLPWQGLKAATKKKKYEKISEKKMSTPVEVLCKGFPAEFAMYLNYCRGLRFEEAPDYMYLRQLFRILFRTLNHQYDYTFDWTMLKQKAAQQAASSSGQGQQAQTPTGKQTDKTKSNMKGF

SEQ ID NO: 51 - DNA, 1011 bp, Synthetic construct
  source 1..1011: mol_type = other DNA
atggcgagtagcagcggctccaaggctgaattcattgtcggagggaaatataaactggtacggaagatcgggtctggctgcttcggggacatctatttggcgatcaacatcaccaacggcgaggaagtggcagtgaagctagaatctcagaaggccaggcatccccagttgctgtacgagagcaagctctataagattcttcaaggtggggttggcatcccccacatacggtggtatggtcaggaaaaagactacaatgtactagtcatggatcttctgggacctagcctcgaagacctcttcaatttctgttcaagaaggttcacaatgaaaactgtacttatgttagctgaccagatgatcagtagaattgaatatgtgcatacaaagaattttatacacagagacattaaaccagataacttcctaatgggtattgggcgtcactgtaataagttattccttattgattttggtttggccaaaaagtacagagacaacaggacaaggcaacacataccatacagagaagataaaaacctcactggcactgcccgatatgctagcatcaatgcacatcttggtattgagcagagtcgccgagatgacatggaatcattaggatatgttttgatgtattttaatagaaccagcctgccatggcaagggctaaaggctgcaacaaagaaaaaaaaatatgaaaagattagtgaaaagaagatgtccacgcctgttgaagttttatgtaaggggtttcctgcagaatttgcgatgtacttaaactattgtcgtgggctacgctttgaggaagccccagattacatgtatctgaggcagctattccgcattcttttcaggaccctgaaccatcaatatgactacacatttgattggacaatgttaaagcagaaagcagcacagcaggcagcctcttccagtgggcagggtcagcaggcccaaacccccacaggcaagcaaactgacaaaaccaagagtaacatgaaaggtttc

SEQ ID NO: 52 - protein, 337 aa, Synthetic construct
MASSSGSKAEFIVGGKYKLVRKIGSGCFGDIYLAINITNGEEVAVKLESQKARHPQLLYESKLYKILQGGVGIPHIRWYGQEKDYNVLVMDLLGPSLEDLFNFCSRRFTMKTVLMLADQMISRIEYVHTKNFIHRDIKPDNFLMGIGRHCNKLFLIDFGLAKKYRDNRTRQHIPYREDKNLTGTARYASINAHLGIEQSRRDDMESLGYVLMYFNRTSLPWQGLKAATKKKKYEKISEKKMSTPVEVLCKGFPAEFAMYLNYCRGLRFEEAPDYMYLRQLFRILFRTLNHQYDYTFDWTMLKQKAAQQAASSSGQGQQAQTPTGKQTDKTKSNMKGF

SEQ ID NO: 53 - DNA, 645 bp, Homo sapiens
  source 1..645: mol_type = genomic DNA
atgagcagctcagaggaggtgtcctggatttcctggttctgtgggctccgtggcaatgaattcttctgtgaagtggatgaagactacatccaggacaaatttaatcttactggactcaatgagcaggtccctcactatcgacaagctctagacatgatcttggacctggagcctgatgaagaactggaagacaaccccaaccagagtgacctgattgagcaggcagccgagatgctttatggattgatccacgcccgctacatccttaccaaccgtggcatcgcccagatgttggaaaagtaccagcaaggagactttggttactgtcctcgtgtgtactgtgagaaccagccaatgcttcccattggcctttcagacatcccaggtgaagccatggtgaagctctactgccccaagtgcatggatgtgtacacacccaagtcatcaagacaccatcacacggatggcgcctacttcggcactggtttccctcacatgctcttcatggtgcatcccgagtaccggcccaagagacctgccaaccagtttgtgcccaggctctacggtttcaagatccatccgatggcctaccagctgcagctccaagccgccagcaacttcaagagcccagtcaagacgattcgc

SEQ ID NO: 54 - protein, 215 aa, Homo sapiens
MSSSEEVSWISWFCGLRGNEFFCEVDEDYIQDKFNLTGLNEQVPHYRQALDMILDLEPDEELEDNPNQSDLIEQAAEMLYGLIHARYILTNRGIAQMLEKYQQGDFGYCPRVYCENQPMLPIGLSDIPGEAMVKLYCPKCMDVYTPKSSRHHHTDGAYFGTGFPHMLFMVHPEYRPKRPANQFVPRLYGFKIHPMAYQLQLQAASNFKSPVKTIR

SEQ ID NO: 55 - DNA, 543 bp, Synthetic construct
  source 1..543: mol_type = other DNA
atgagcagctcagaggaggtgtcctggatttcctggttctgtgggctccgtggcaatgaattcttctgtgaagtggatgaagactacatccaggacaaatttaatcttactggactcaatgagcaggtccctcactatcgacaagctctagacatgatcttggacctggagcctgatgaagaactggaagacaaccccaaccagagtgacctgattgagcaggcagccgagatgctttatggattgatccacgcccgctacatccttaccaaccgtggcatcgcccagatgttggaaaagtaccagcaaggagactttggttactgtcctcgtgtgtactgtgagaaccagccaatgcttcccattggcctttcagacatcccaggtgaagccatggtgaagctctactgccccaagtgcatggatgtgtacacacccaagtcatcaagacaccatcacacggatggcgcctacttcggcactggtttccctcacatgctcttcatggtgcatcccgagtaccggcccaagagacctgccaac

SEQ ID NO: 56 - protein, 181 aa, Synthetic construct
MSSSEEVSWISWFCGLRGNEFFCEVDEDYIQDKFNLTGLNEQVPHYRQALDMILDLEPDEELEDNPNQSDLIEQAAEMLYGLIHARYILTNRGIAQMLEKYQQGDFGYCPRVYCENQPMLPIGLSDIPGEAMVKLYCPKCMDVYTPKSSRHHHTDGAYFGTGFPHMLFMVHPEYRPKRPAN

SEQ ID NO: 57 - DNA, 1293 bp, Synthetic construct
  source 1..1293: mol_type = other DNA
atgagctgggtccaggccaccctactggcccgaggcctctgtagggcctggggaggcacctgcggggccgccctcacaggaacctccatctctcaggtccctcgccggctccctcggggcctccactgcagcgcagctgcccatagctctgaacagtccctggttcccagcccaccggaaccccggcagaggcccaccaaggctctggtgccctttgaggacctgtttgggcaggcgcctggtggggaacgggacaaggcgagcttcctgcagacggtgcagaaatttgcggagcacagcgtgcgtaagcggggccacattgacttcatctacctggccctgcgcaagatgcgggagtatggtgtcgagcgggacctggctgtgtacaaccagctgctcaacatcttccccaaggaggccttccggcctcgcaacatcatccagcgcatcttcgtccactaccctcggcagcaggagtgtgggattgctgtcctggagcagatggagaaccacggtgtgatgcccaacaaggagacggagttcctgctgattcagatctttggacgcaaaagctaccccatgctcaagttggtgcgcctgaagctgtggttccctcgattcatgaacgtcaaccccttcccagtgccccgggacctgccccaggaccctgtggagctggccatgtttggcctgcggcacatggagcctgaccttagtgccagggtcaccatctaccaggttcctttgcccaaagactcaacaggtgcagcagatcccccccagccccacatcgtaggaatccagagtcccgatcagcaggccgccctggcccgccacaatccagcccggcctgtctttgttgagggccccttctccctgtggctccgcaacaagtgtgtgtattaccacatcctcagagctgacttgctgcccccggaggagagggaagtggaagagacgccggaggagtggaacctctactacccgatgcagctggacctggagtatgtgaggagtggctgggacaactacgagtttgacatcaatgaagtggaggaaggccctgtcttcgccatgtgcatggcgggtgctcatgaccaggcgacgatggctaagtggatccagggcctgcaggagaccaacccaaccctggcccagatccccgtggtcttccgcctcgccgggtccacccgggagctccagacatcctctgcagggctggaggagccgcccctgcccgaggaccaccaggaagaagacgacaacctgcagcgacagcagcagggccagagc

SEQ ID NO: 58 - protein, 431 aa, Synthetic construct [sequence omitted]

SEQ ID NO: 59 - DNA, 1293 bp, Homo sapiens
  source 1..1293: mol_type = genomic DNA
atgagctgggtccaggccaccctactggcccgaggcctctgtagggcctggggaggcacctgcggggccgccctcacaggaacctccatctctcaggtccctcgccggctccctcggggcctccactgcagcgcagctgcccatagctctgaacagtccctggttcccagcccaccggaaccccggcagaggcccaccaaggctctggtgccctttgaggacctgtttgggcaggcgcctggtggggaacgggacaaggcgagcttcctgcagacggtgcagaaatttgcggagcacagcgtgcgtaagcggggccacattgacttcatctacctggccctgcgcaagatgcgggagtatggtgtcgagcgggacctggctgtgtacaaccagctgctcaacatcttccccaaggaggtcttccggcctcgcaacatcatccagcgcatcttcgtccactaccctcggcagcaggagtgtgggattgctgtcctggagcagatggagaaccacggtgtgatgcccaacaaggagacggagttcctgctgattcagatctttggacgcaaaagctaccccatgctcaagttggtgcgcctgaagctgtggttccctcgattcatgaacgtcaaccccttcccagtgccccgggacctgccccaggaccctgtggagctggccatgtttggcctgcggcacatggagcctgaccttagtgccagggtcaccatctaccaggttcctttgcccaaagactcaacaggtgcagcagatcccccccagccccacatcgtaggaatccagagtcccgatcagcaggccgccctggcccgccacaatccagcccggcctgtctttgttgagggccccttctccctgtggctccgcaacaagtgtgtgtattaccacatcctcagagctgacttgctgcccccggaggagagggaagtggaagagacgccggaggagtggaacctctactacccgatgcagctggacctggagtatgtgaggagtggctgggacaactacgagtttgacatcaatgaagtggaggaaggccctgtcttcgccatgtgcatggcgggtgctcatgaccaggcgacgatggctaagtggatccagggcctgcaggagaccaacccaaccctggcccagatccccgtggtcttccgcctcgccgggtccacccgggagctccagacatcctctgcagggctggaggagccgcccctgcccgaggaccaccaggaagaagacgacaacctgcagcgacagcagcagggccagagc

SEQ ID NO: 60 - protein, 431 aa, Homo sapiens
MSWVQATLLARGLCRAWGGTCGAALTGTSISQVPRRLPRGLHCSAAAHSSEQSLVPSPPEPRQRPTKALVPFEDLFGQAPGGERDKASFLQTVQKFAEHSVRKRGHIDFIYLALRKMREYGVERDLAVYNQLLNIFPKEVFRPRNIIQRIFVHYPRQQECGIAVLEQMENHGVMPNKETEFLLIQIFGRKSYPMLKLVRLKLWFPRFMNVNPFPVPRDLPQDPVELAMFGLRHMEPDLSARVTIYQVPLPKDSTGAADPPQPHIVGIQSPDQQAALARHNPARPVFVEGPFSLWLRNKCVYYHILRADLLPPEEREVEETPEEWNLYYPMQLDLEYVRSGWDNYEFDINEVEEGPVFAMCMAGAHDQATMAKWIQGLQETNPTLAQIPVVFRLAGSTRELQTSSAGLEEPPLPEDHQEEDDNLQRQQQGQS

SEQ ID NO: 61 - DNA, 945 bp, Synthetic construct
  source 1..945: mol_type = other DNA
atgccgggtctaagttgtagattttatcaacacaaatttcctgaggtggaagatgtagtgatggtgaatgtcagatccattgctgaaatgggggcttatgtcagcttgctggaatacaacaacattgaaggcatgattcttcttagtgaattatccagaaggcgtatccgttctatcaacaaactcatccgaattggcaggaatgagtgtgtggttgtcattagggtggacaaagaaaaaggatatattgatttgtcaaaaagaatcgtttctccagaggaagcaatcaaatgtgaagacaaattcacaaaatccaaaactgtttatagcattcttcgtcatgttgctgaggtgttagaatacaccaaggatgagcagctggaaagcctattccagaggactgcctgggtctttgatgacaagtacaagagacctggatatggtgcctatgatgcatttaagcatgcagtctcagacccatctattttggatagtttagatttgaatgaagatgaacgggaagtactcattaataatattaataggcgcttgaccccacaggctgtcaaaattcgagcagatattgaagtggcttgttatggttatgaaggcattgatgctgtaaaagaagccctaagagcaggtttgaattgttctacagaaaacatgcccattaagattaatctaatagctcctcctcggtatgtaatgactacgacaaccctggagagaacagaaggcctttctgtcctcagtcaagctatggctgttatcaaagagaagattgaggaaaagaggggtgtgttcaatgttcaaatggagcccaaagtggtcacagatacagatgagactgaacttgcgaggcagatggagaggcttgaaagagaaaatgccgaagtggatggagatgatgatgcagaagaaatggaagccaaagctgaagat

SEQ ID NO: 62 - protein, 315 aa, Synthetic construct
MPGLSCRFYQHKFPEVEDVVMVNVRSIAEMGAYVSLLEYNNIEGMILLSELSRRRIRSINKLIRIGRNECVVVIRVDKEKGYIDLSKRIVSPEEAIKCEDKFTKSKTVYSILRHVAEVLEYTKDEQLESLFQRTAWVFDDKYKRPGYGAYDAFKHAVSDPSILDSLDLNEDEREVLINNINRRLTPQAVKIRADIEVACYGYEGIDAVKEALRAGLNCSTENMPIKINLIAPPRYVMTTTTLERTEGLSVLSQAMAVIKEKIEEKRGVFNVQMEPKVVTDTDETELARQMERLERENAEVDGDDDAEEMEAKAED

SEQ ID NO: 63 - DNA, 945 bp, Homo sapiens
  source 1..945: mol_type = genomic DNA
atgccgggtctaagttgtagattttatcaacacaaatttcctgaggtggaagatgtagtgatggtgaatgtcagatccattgctgaaatgggggcttatgtcagcttgctggaatacaacaacattgaaggcatgattcttcttagtgaattatccagaaggcgtatccgttctatcaacaaactcatccgaattggcaggaatgagtgtgtggttgtcattagggtggacaaagaaaaaggatatattgatttgtcaaaaagaagagtttctccagaggaagcaatcaaatgtgaagacaaattcacaaaatccaaaactgtttatagcattcttcgtcatgttgctgaggtgttagaatacaccaaggatgagcagctggaaagcctattccagaggactgcctgggtctttgatgacaagtacaagagacctggatatggtgcctatgatgcatttaagcatgcagtctcagacccatctattttggatagtttagatttgaatgaagatgaacgggaagtactcattaataatattaataggcgcttgaccccacaggctgtcaaaattcgagcagatattgaagtggcttgttatggttatgaaggcattgatgctgtaaaagaagccctaagagcaggtttgaattgttctacagaaaacatgcccattaagattaatctaatagctcctcctcggtatgtaatgactacgacaaccctggagagaacagaaggcctttctgtcctcagtcaagctatggctgttatcaaagagaagattgaggaaaagaggggtgtgttcaatgttcaaatggagcccaaagtggtcacagatacagatgagactgaacttgcgaggcagatggagaggcttgaaagagaaaatgccgaagtggatggagatgatgatgcagaagaaatggaagccaaagctgaagat

SEQ ID NO: 64 - protein, 315 aa, Homo sapiens
MPGLSCRFYQHKFPEVEDVVMVNVRSIAEMGAYVSLLEYNNIEGMILLSELSRRRIRSINKLIRIGRNECVVVIRVDKEKGYIDLSKRRVSPEEAIKCEDKFTKSKTVYSILRHVAEVLEYTKDEQLESLFQRTAWVFDDKYKRPGYGAYDAFKHAVSDPSILDSLDLNEDEREVLINNINRRLTPQAVKIRADIEVACYGYEGIDAVKEALRAGLNCSTENMPIKINLIAPPRYVMTTTTLERTEGLSVLSQAMAVIKEKIEEKRGVFNVQMEPKVVTDTDETELARQMERLERENAEVDGDDDAEEMEAKAED

SEQ ID NO: 65 - DNA, 1614 bp, Synthetic construct
  source 1..1614: mol_type = other DNA
atgggctgtgtgcaatgtaaggataaagaagcaacaaaactgacggaggagagggacggcagcctgaaccagagctctgggtaccgctatggcacagaccccacccctcagcactaccccagcttcggtgtgacctccatccccaactacaacaacttccacgcagccgggggccaaggactcaccgtctttggaggtgtgaactcttcgtctcatacggggaccttgcgtacgagaggaggaacaggagtgacactctttgtggccctttatgactatgaagcagggacagaagatgacctgagttttcacaaaggagaaaaatttcaaatattgaacagctcggaaggagattggtgggaagcccgctccttgacaactggagagacaggttacattcccagcaattatgtggctccagttgactctatccaggcagaagagtggtactttggaaaacttggccgaaaagatgctgagcgacagctattgtcctttggaaacccaagaggtacctttcttatccgcgagagtgaaaccaccaaaggtgcctattcactttctatccgtgattgggatgatatgaaaggagaccatgtcaaacattataaaattcgcaaacttgacaatggtggatactacattaccacccgggcccagtttgaaacacttcagcagcttgtacaacattactcagagagagctgcaggtctctgctgccgcctagtagttccctgtcacaaagggatgccaaggcttaccgatctgtctgtcaaaaccaaagatgtctgggaaatccctcgagaatccctgcagttgatcaagagactgggaaatgggcagtttggggaagtatggatgggtacctggaatggaaacacaaaagtagccataaagactcttaaaccaggcacaatgtcccccgaatcattccttgaggaagcgcagatcatgaagaagctgaagcacgacaagctggtccagctctatgcagtggtgtctgaggagcccatctacatcgtcaccgagtatatgaacaaaggaagtttactggatttcttaaaagatggagaaggaagagctctgaaattaccaaatcttgtggacatggcagcacaggtggctgcaggaatggcttacatcgagcgcatgaattatatccatagagatctgcgatcagcaaacattctagtggggaatggactcatatgcaagattgctgacttcggattggcccgattgatagaagacaatgagtacacagcaagacaaggtgcaaagttccccatcaagtggacggcccccgaggcagccctgtacgggaggttcacaatcaagtctgacgtgtggtcttttggaatcttactcacagagctggtcaccaaaggaagagtgccatacccaggcatgaacaaccgggaggtgctggagcaggtggagcgaggctacaggatgccctgcccgcaggactgccccatctctctgcatgagctcatgatccactgctggaaaaaggaccctgaagaacgccccacttttgagtacttgcagagcttcctggaagactactttaccgcgacagagccccagtacaaacctggtgaaaacctgttg

SEQ ID NO: 66 - protein, 538 aa, Synthetic construct
MGCVQCKDKEATKLTEERDGSLNQSSGYRYGTDPTPQHYPSFGVTSIPNYNNFHAAGGQGLTVFGGVNSSSHTGTLRTRGGTGVTLFVALYDYEAGTEDDLSFHKGEKFQILNSSEGDWWEARSLTTGETGYIPSNYVAPVDSIQAEEWYFGKLGRKDAERQLLSFGNPRGTFLIRESETTKGAYSLSIRDWDDMKGDHVKHYKIRKLDNGGYYITTRAQFETLQQLVQHYSERAAGLCCRLVVPCHKGMPRLTDLSVKTKDVWEIPRESLQLIKRLGNGQFGEVWMGTWNGNTKVAIKTLKPGTMSPESFLEEAQIMKKLKHDKLVQLYAVVSEEPIYIVTEYMNKGSLLDFLKDGEGRALKLPNLVDMAAQVAAGMAYIERMNYIHRDLRSANILVGNGLICKIADFGLARLIEDNEYTARQGAKFPIKWTAPEAALYGRFTIKSDVWSFGILLTELVTKGRVPYPGMNNREVLEQVERGYRMPCPQDCPISLHELMIHCWKKDPEERPTFEYLQSFLEDYFTATEPQYKPGENLL

SEQ ID NO: 67 - DNA, 1614 bp, Homo sapiens
  source 1..1614: mol_type = genomic DNA
atgggctgtgtgcaatgtaaggataaagaagcaacaaaactgacggaggagagggacggcagcctgaaccagagctctgggtaccgctatggcacagaccccacccctcagcactaccccagcttcggtgtgacctccatccccaactacaacaacttccacgcagccgggggccaaggactcaccgtctttggaggtgtgaactcttcgtctcatacggggaccttgcgtacgagaggaggaacaggagtgacactctttgtggccctttatgactatgaagcacggacagaagatgacctgagttttcacaaaggagaaaaatttcaaatattgaacagctcggaaggagattggtgggaagcccgctccttgacaactggagagacaggttacattcccagcaattatgtggctccagttgactctatccaggcagaagagtggtactttggaaaacttggccgaaaagatgctgagcgacagctattgtcctttggaaacccaagaggtacctttcttatccgcgagagtgaaaccaccaaaggtgcctattcactttctatccgtgattgggatgatatgaaaggagaccatgtcaaacattataaaattcgcaaacttgacaatggtggatactacattaccacccgggcccagtttgaaacacttcagcagcttgtacaacattactcagagagagctgcaggtctctgctgccgcctagtagttccctgtcacaaagggatgccaaggcttaccgatctgtctgtcaaaaccaaagatgtctgggaaatccctcgagaatccctgcagttgatcaagagactgggaaatgggcagtttggggaagtatggatgggtacctggaatggaaacacaaaagtagccataaagactcttaaaccaggcacaatgtcccccgaatcattccttgaggaagcgcagatcatgaagaagctgaagcacgacaagctggtccagctctatgcagtggtgtctgaggagcccatctacatcgtcaccgagtatatgaacaaaggaagtttactggatttcttaaaagatggagaaggaagagctctgaaattaccaaatcttgtggacatggcagcacaggtggctgcaggaatggcttacatcgagcgcatgaattatatccatagagatctgcgatcagcaaacattctagtggggaatggactcatatgcaagattgctgacttcggattggcccgattgatagaagacaatgagtacacagcaagacaaggtgcaaagttccccatcaagtggacggcccccgaggcagccctgtacgggaggttcacaatcaagtctgacgtgtggtcttttggaatcttactcacagagctggtcaccaaaggaagagtgccatacccaggcatgaacaaccgggaggtgctggagcaggtggagcgaggctacaggatgccctgcccgcaggactgccccatctctctgcatgagctcatgatccactgctggaaaaaggaccctgaagaacgccccacttttgagtacttgcagagcttcctggaagactactttaccgcgacagagccccagtacaaacctggtgaaaacctgttg

SEQ ID NO: 68 - protein, 538 aa, Homo sapiens
MGCVQCKDKEATKLTEERDGSLNQSSGYRYGTDPTPQHYPSFGVTSIPNYNNFHAAGGQGLTVFGGVNSSSHTGTLRTRGGTGVTLFVALYDYEARTEDDLSFHKGEKFQILNSSEGDWWEARSLTTGETGYIPSNYVAPVDSIQAEEWYFGKLGRKDAERQLLSFGNPRGTFLIRESETTKGAYSLSIRDWDDMKGDHVKHYKIRKLDNGGYYITTRAQFETLQQLVQHYSERAAGLCCRLVVPCHKGMPRLTDLSVKTKDVWEIPRESLQLIKRLGNGQFGEVWMGTWNGNTKVAIKTLKPGTMSPESFLEEAQIMKKLKHDKLVQLYAVVSEEPIYIVTEYMNKGSLLDFLKDGEGRALKLPNLVDMAAQVAAGMAYIERMNYIHRDLRSANILVGNGLICKIADFGLARLIEDNEYTARQGAKFPIKWTAPEAALYGRFTIKSDVWSFGILLTELVTKGRVPYPGMNNREVLEQVERGYRMPCPQDCPISLHELMIHCWKKDPEERPTFEYLQSFLEDYFTATEPQYKPGENLL

SEQ ID NO: 69 - DNA, 1614 bp, Synthetic construct
  source 1..1614: mol_type = other DNA
atgggctgtgtgcaatgtaaggataaagaagcaacaaaactgacggaggagagggacggcagcctgaaccagagctctgggtaccgctatggcacagaccccacccctcagcactaccccagcttcggtgtgacctccatccccaactacaacaacttccacgcagccgggggccaaggactcaccgtctttggaggtgtgaactcttcgtctcatacggggaccttgcgtacgagaggaggaacaggagtgacactctttgtggccctttatgactatgaagcacggacagaagatgacctgagttttcacaaaggagaaaaatttcaaatattgaacagctcggaaggagattggtgggaagcccgctccttgacaactggagagacaggttacattcccagcaattatgtggctccagttgactctatccaggcagaagagtggtactttggaaaacttggccgaaaagatgctgagcgacagctattgtcctttggaaacccaagaggtacctttcttatccgcgagagtgaaaccaccaaaggtgcctattcactttctatccgtgattgggatgatatgaaaggagaccatgtcaaacattataaaattcgcaaacttgacaatggtggatactacattaccacccgggcccagtttgaaacacttcagcagcttgtacaacattactcagagagagctgcaggtctctgctgccgcctagtagttccctgtcacaaagggatgccaaggcttaccgatctgtctgtcaaaaccaaagatgtctgggaaatccctcgagaatccctgcagttgatcaagagactgggaaatgggcagtttggggaagtatggatgggtacctggaatggaaacacaaaagtagccataaagactcttaaaccaggcacaatgtcccccgaatcattccttgaggaagcgcagatcatgaagaagctgaagcacgacaagctggtccagctctatgcagtggtgtctgaggagcccatctacatcgtcaccgagtatatgaacaaaggaagtttactggatttcttaaaagatggagaaggaagagctctgaaattaccaaatcttgtggacatggcagcacaggtggctgcaggaatggcttacatcgagcgcatgaattatatccatagagatctgcgatcagcaaacattctagtggggaatggactcatatgcaagattgctgacttcggattggcccgattgatagaagacaatgagtacacagcaagacaaggtgcaaagttccccatcaagtggacggcccccgaggcagccctgtacgggaggttcacaatcaagtctgacgtgtggtcttttggaatcttactcacagagctggtcaccaaaggaagagtgccatacccaggcatgaacaaccgggaggtgctggagcaggtggagcgaggctacaggatgccctgcccgcaggactgccccatctctctgcatgagctcatgatccactgctggaaaaaggaccctgaagaacgccccacttttgagtacttgcagagcttcctggaagactactttaccgcgacagagccccagcacaaacctggtgaaaacctgttg

SEQ ID NO: 70 - protein, 538 aa, Synthetic construct
MGCVQCKDKEATKLTEERDGSLNQSSGYRYGTDPTPQHYPSFGVTSIPNYNNFHAAGGQGLTVFGGVNSSSHTGTLRTRGGTGVTLFVALYDYEARTEDDLSFHKGEKFQILNSSEGDWWEARSLTTGETGYIPSNYVAPVDSIQAEEWYFGKLGRKDAERQLLSFGNPRGTFLIRESETTKGAYSLSIRDWDDMKGDHVKHYKIRKLDNGGYYITTRAQFETLQQLVQHYSERAAGLCCRLVVPCHKGMPRLTDLSVKTKDVWEIPRESLQLIKRLGNGQFGEVWMGTWNGNTKVAIKTLKPGTMSPESFLEEAQIMKKLKHDKLVQLYAVVSEEPIYIVTEYMNKGSLLDFLKDGEGRALKLPNLVDMAAQVAAGMAYIERMNYIHRDLRSANILVGNGLICKIADFGLARLIEDNEYTARQGAKFPIKWTAPEAALYGRFTIKSDVWSFGILLTELVTKGRVPYPGMNNREVLEQVERGYRMPCPQDCPISLHELMIHCWKKDPEERPTFEYLQSFLEDYFTATEPQHKPGENLL

SEQ ID NO: 71 - DNA, 186 bp, Synthetic construct
  source 1..186: mol_type = other DNA
atggaggtgacggcggaccagccgcgctgggtgagccaccaccaccccgccgtgctcaacgggcagcacccggacacgcaccacccgggcctcagccactcctacatggacgcggcgcagtacccgctgccggaggaggtggatgtgctttttaacatcgacggtcaaggcaaccacgtcccgccc

SEQ ID NO: 72 - protein, 62 aa, Synthetic construct
MEVTADQPRWVSHHHPAVLNGQHPDTHHPGLSHSYMDAAQYPLPEEVDVLFNIDGQGNHVPP

SEQ ID NO: 73 - DNA, 1332 bp, Homo sapiens
  source 1..1332: mol_type = genomic DNA
atggaggtgacggcggaccagccgcgctgggtgagccaccaccaccccgccgtgctcaacgggcagcacccggacacgcaccacccgggcctcagccactcctacatggacgcggcgcagtacccgctgccggaggaggtggatgtgctttttaacatcgacggtcaaggcaaccacgtcccgccctactacggaaactcggtcagggccacggtgcagaggtaccctccgacccaccacgggagccaggtgtgccgcccgcctctgcttcatggatccctaccctggctggacggcggcaaagccctgggcagccaccacaccgcctccccctggaatctcagccccttctccaagacgtccatccaccacggctccccggggcccctctccgtctaccccccggcctcgtcctcctccttgtcggggggccacgccagcccgcacctcttcaccttcccgcccaccccgccgaaggacgtctccccggacccatcgctgtccaccccaggctcggccggctcggcccggcaggacgagaaagagtgcctcaagtaccaggtgcccctgcccgacagcatgaagctggagtcgtcccactcccgtggcagcatgaccgccctgggtggagcctcctcgtcgacccaccaccccatcaccacctacccgccctacgtgcccgagtacagctccggactcttcccccccagcagcctgctgggcggctcccccaccggcttcggatgcaagtccaggcccaaggcccggtccagcacagaaggcagggagtgtgtgaactgtggggcaacctcgaccccactgtggcggcgagatggcacgggacactacctgtgcaacgcctgcgggctctatcacaaaatgaacggacagaaccggcccctcattaagcccaagcgaaggctgtctgcagccaggagagcagggacgtcctgtgcgaactgtcagaccaccacaaccacactctggaggaggaatgccaatggggaccctgtctgcaatgcctgtgggctctactacaagcttcacaatattaacagacccctgactatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagcctgtcctttggaccacaccacccctccagcatggtcaccgccatgggt

SEQ ID NO: 74 - protein, 444 aa, Homo sapiens
MEVTADQPRWVSHHHPAVLNGQHPDTHHPGLSHSYMDAAQYPLPEEVDVLFNIDGQGNHVPPYYGNSVRATVQRYPPTHHGSQVCRPPLLHGSLPWLDGGKALGSHHTASPWNLSPFSKTSIHHGSPGPLSVYPPASSSSLSGGHASPHLFTFPPTPPKDVSPDPSLSTPGSAGSARQDEKECLKYQVPLPDSMKLESSHSRGSMTALGGASSSTHHPITTYPPYVPEYSSGLFPPSSLLGGSPTGFGCKSRPKARSSTEGRECVNCGATSTPLWRRDGTGHYLCNACGLYHKMNGQNRPLIKPKRRLSAARRAGTSCANCQTTTTTLWRRNANGDPVCNACGLYYKLHNINRPLTMKKEGIQTRNRKMSSKSKKCKKVHDSLEDFPKNSSFNPAALSRHMSSLSHISPFSHSSHMLTTPTPMHPPSSLSFGPHHPSSMVTAMG

SEQ ID NO: 75 - DNA, 1077 bp, Synthetic construct
  source 1..1077: mol_type = other DNA
atgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacagcctcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggagtacaatctggtc

SEQ ID NO: 76 - protein, 359 aa, Synthetic construct
MTLESIMACCLSEEAKEARRINDEIERQLRRDKRDARRELKLLLLGTGESGKSTFIKQMRIIHGSGYSDEDKRGFTKLVYQNIFTAMQAMIRAMDSLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKEYNLV

SEQ ID NO: 77 - DNA, 1077 bp, Homo sapiens
  source 1..1077: mol_type = genomic DNA
atgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggagtacaatctggtc

SEQ ID NO: 78 - protein, 359 aa, Homo sapiens
MTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKEYNLV

SEQ ID NO: 79 - DNA, 1356 bp, Synthetic construct
  source 1..1356: mol_type = other DNA
atgaacctggagggcggcggccgaggcggagagttcggcatgagcgcggtgagctgcggcaacgggaagctccgccagtggctgatcgaccagatcgacagcggcaagtaccccgggctggtgtgggagaacgaggagaagagcatcttccgcatcccctggaagcacgcgggcaggcaggactacaaccgcgaggaggacgccgcgctcttcaaggcttgggcactgtttaaaggaaagttccgagaaggcatcgacaagccggaccctcccacctggaagacgcgcctgcggtgcgctttgaacaagagcaatgactttgaggaactggttgagcggagccagctggacatctcagacccgtacaaagtgtacaggattgttcctgagggagccaaaaaaggagccaagcagctcaccttggaggacccgcagatgtccatgagccacccctacaccatgacaacgccttacccttcgctcccagcccagcaggttcacaactacatgatgccacccctcgaccgaagctggagggactacgtcccggatcagccacacccggaaatcccgtaccaatgtcccatgacgtttggaccccgcggccaccactggcaaggcccagcttgtgaaaatggttgccaggtgacaggaaccttttatgcttgtgccccacctgagtcccaggctcccggagtccccacagagccaagcataaggtctgccgaagccttggcgttctcagactgccggctgcacatctgcctgtactaccgggaaatcctcgtgaaggagctgaccacgtccagccccgagggctgccggatctcccatggacatacgtatgacgccagcaacctggaccaggtcctgttcccctacccagaggacaatggccagaggaaaaacattgagaagctgctgagccacctggagaggggcgtggtcctctggatggcccccgacgggctctatgcgaaaagactgtgccagagcaggatctactgggacgggcccctggcgctgtgcaacgaccggcccaacaaactggagagagaccagacctgcaagctctttgacacacagcagttcttgtcagagctgcaagcgtttgctcaccacggccgctccctgccaagattccaggtgactctatgctttggagaggagtttccagaccctcagaggcaaagaaagctcatcacagctcacgtagaacctctgctagccagacaactatattattttgctcaacaaaacagtggacatttcctgaggggctacgatttaccagaacacatcagcaatccagaagattaccacagatctatccgccattcctctattcaagaattg

SEQ ID NO: 80 - protein, 452 aa, Synthetic construct
MNLEGGGRGGEFGMSAVSCGNGKLRQWLIDQIDSGKYPGLVWENEEKSIFRIPWKHAGRQDYNREEDAALFKAWALFKGKFREGIDKPDPPTWKTRLRCALNKSNDFEELVERSQLDISDPYKVYRIVPEGAKKGAKQLTLEDPQMSMSHPYTMTTPYPSLPAQQVHNYMMPPLDRSWRDYVPDQPHPEIPYQCPMTFGPRGHHWQGPACENGCQVTGTFYACAPPESQAPGVPTEPSIRSAEALAFSDCRLHICLYYREILVKELTTSSPEGCRISHGHTYDASNLDQVLFPYPEDNGQRKNIEKLLSHLERGVVLWMAPDGLYAKRLCQSRIYWDGPLALCNDRPNKLERDQTCKLFDTQQFLSELQAFAHHGRSLPRFQVTLCFGEEFPDPQRQRKLITAHVEPLLARQLYYFAQQNSGHFLRGYDLPEHISNPEDYHRSIRHSSIQEL

SEQ ID NO: 81 - DNA, 1356 bp, Homo sapiens
  source 1..1356: mol_type = genomic DNA
atgaacctggagggcggcggccgaggcggagagttcggcatgagcgcggtgagctgcggcaacgggaagctccgccagtggctgatcgaccagatcgacagcggcaagtaccccgggctggtgtgggagaacgaggagaagagcatcttccgcatcccctggaagcacgcgggcaagcaggactacaaccgcgaggaggacgccgcgctcttcaaggcttgggcactgtttaaaggaaagttccgagaaggcatcgacaagccggaccctcccacctggaagacgcgcctgcggtgcgctttgaacaagagcaatgactttgaggaactggttgagcggagccagctggacatctcagacccgtacaaagtgtacaggattgttcctgagggagccaaaaaaggagccaagcagctcaccttggaggacccgcagatgtccatgagccacccctacaccatgacaacgccttacccttcgctcccagcccagcaggttcacaactacatgatgccacccctcgaccgaagctggagggactacgtcccggatcagccacacccggaaatcccgtaccaatgtcccatgacgtttggaccccgcggccaccactggcaaggcccagcttgtgaaaatggttgccaggtgacaggaaccttttatgcttgtgccccacctgagtcccaggctcccggagtccccacagagccaagcataaggtctgccgaagccttggcgttctcagactgccggctgcacatctgcctgtactaccgggaaatcctcgtgaaggagctgaccacgtccagccccgagggctgccggatctcccatggacatacgtatgacgccagcaacctggaccaggtcctgttcccctacccagaggacaatggccagaggaaaaacattgagaagctgctgagccacctggagaggggcgtggtcctctggatggcccccgacgggctctatgcgaaaagactgtgccagagcaggatctactgggacgggcccctggcgctgtgcaacgaccggcccaacaaactggagagagaccagacctgcaagctctttgacacacagcagttcttgtcagagctgcaagcgtttgctcaccacggccgctccctgccaagattccaggtgactctatgctttggagaggagtttccagaccctcagaggcaaagaaagctcatcacagctcacgtagaacctctgctagccagacaactatattattttgctcaacaaaacagtggacatttcctgaggggctacgatttaccagaacacatcagcaatccagaagattaccacagatctatccgccattcctctattcaagaattg

SEQ ID NO: 82 - protein, 452 aa, Homo sapiens
MNLEGGGRGGEFGMSAVSCGNGKLRQWLIDQIDSGKYPGLVWENEEKSIFRIPWKHAGKQDYNREEDAALFKAWALFKGKFREGIDKPDPPTWKTRLRCALNKSNDFEELVERSQLDISDPYKVYRIVPEGAKKGAKQLTLEDPQMSMSHPYTMTTPYPSLPAQQVHNYMMPPLDRSWRDYVPDQPHPEIPYQCPMTFGPRGHHWQGPACENGCQVTGTFYACAPPESQAPGVPTEPSIRSAEALAFSDCRLHICLYYREILVKELTTSSPEGCRISHGHTYDASNLDQVLFPYPEDNGQRKNIEKLLSHLERGVVLWMAPDGLYAKRLCQSRIYWDGPLALCNDRPNKLERDQTCKLFDTQQFLSELQAFAHHGRSLPRFQVTLCFGEEFPDPQRQRKLITAHVEPLLARQLYYFAQQNSGHFLRGYDLPEHISNPEDYHRSIRHSSIQEL

SEQ ID NO: 83 - DNA, 2307 bp, Synthetic construct
  source 1..2307: mol_type = other DNA
atgctgggcctgcgccccccacttctcgccctggtggggctgctctccctcgggtgcgtcctctctcaggagtgcacgaagttcaaggtcagcagctgccgggaatgcatcgagtcggggcccggctgcacctggtgccagaagctgaacttcacagggccgggggatcctgactccattcgctgcgacacccggccacagctgctcatgaggggctgtgcggctgacgacatcatggaccccacaagcctcgctgaaacccaggaagaccacaatgggggccagaagcagctgtccccacaaaaagtgacgctttacctgcgaccaggccaggcagcagcgttcaacgtgaccttccggcgggccaagggctaccccatcgacctgtactatctgatggacctctcctactccatgcttgatgacctcaggaatgtcaagaagctaggtggcgacctgctccgggccctcaacgagatcaccgagtccggccgcattggcttcgggtccttcgtggacaagaccgtgctgccgttcgtgaacacgcaccctgataagctgcgaaacccatgccccaacaaggagaaagagtgccagcccccgtttgccttcaggcacgtgctgaagctgaccaacaactccaaccagtttcagaccgaggtcgggaagcagctgatttccggaaacctggatgcacccaagggtgggctggacgccatgatgcaggtcgccgcctgcccggaggaaatcggctggcgcaacgtcacgcggctgctggtgtttgccactgatgacggcttccatttcgcgggcgacgggaagctgggcgccatcctgacccccaacgacggccgctgtcacctggaggacaacttgtacaagaggagcaacgaattcgactacccatcggtgggccagctggcgcacaagctggctgaaaacaacatccagcccatcttcgcggtgaccagtaggatggtgaagacctacgagaaactcaccgagatcatccccaagtcagccgtgggggagctgtctgaggactccagcaatgtggtccatctcattaagaatgcttacaataaactctcctccagggtattcctggatcacaacgccctccccgacaccctgaaagtcacctacgactccttctgcagcaatggagtgacgcacaggaaccagcccagaggtgactgtgatggcgtgcagatcaatgtcccgatcaccttccaggtgaaggtcacggccacagagtgcatccaggagcagtcgtttgtcatccgggcgctgggcttcacggacatagtgaccgtgcaggtccttccccagtgtgagtgccggtgccgggaccagagcagagaccgcagcctctgccatggcaagggcttcttggagtgcggcatctgcaggtgtgacactggctacattgggaaaaactgtgagtgccagacacagggccggagcagccaggagctggaaggaagctgccggaaggacaacaactccatcatctgctcagggctgggggactgtgtctgcgggcagtgcctgtgccacaccagcgacgtccccggcaagctgatatacgggcagtactgcgagtgtgacaccatcaactgtgagcgctacaacggccaggtctgcggcggcccggggagggggctctgcttctgcgggaagtgccgctgccacccgggctttgagggctcagcgtgccagtgcgagaggaccactgagggctgcctgaacccgcggcgtgttgagtgtagtggtcgtggccggtgccgctgcaacgtatgcgagtgccattcaggctaccagctgcctctgtgccaggagtgccccggctgcccctcaccctgtggcaagtacatctcctgcgccgagtgcctgaagttcgaaaagggcccctttgggaagaactgcagcgcggcgtgtccgggcctgcagctgtcgaacaaccccgtgaagggcaggacctgcaaggagagggactcagagggctgctgggtggcctacacgctggagcagcaggacgggatggaccgctacctcatctatgtggatgagagccgagagtgtgtggcaggccccaacatcgccgccatcgtcgggggcaccgtggcaggcatcgtgctgatcggcattctcctgctggtcatctggaaggctctgatccacctgagcgacctccgggagtacaggcgctttgagaaggagaagctcaagtcccagtggaacaatgataatccccttttcaagagcgccaccacgacggtcatgaaccccaagtttgctgagagt

SEQ ID NO: 84 - protein, 769 aa, Synthetic construct
MLGLRPPLLALVGLLSLGCVLSQECTKFKVSSCRECIESGPGCTWCQKLNFTGPGDPDSIRCDTRPQLLMRGCAADDIMDPTSLAETQEDHNGGQKQLSPQKVTLYLRPGQAAAFNVTFRRAKGYPIDLYYLMDLSYSMLDDLRNVKKLGGDLLRALNEITESGRIGFGSFVDKTVLPFVNTHPDKLRNPCPNKEKECQPPFAFRHVLKLTNNSNQFQTEVGKQLISGNLDAPKGGLDAMMQVAACPEEIGWRNVTRLLVFATDDGFHFAGDGKLGAILTPNDGRCHLEDNLYKRSNEFDYPSVGQLAHKLAENNIQPIFAVTSRMVKTYEKLTEIIPKSAVGELSEDSSNVVHLIKNAYNKLSSRVFLDHNALPDTLKVTYDSFCSNGVTHRNQPRGDCDGVQINVPITFQVKVTATECIQEQSFVIRALGFTDIVTVQVLPQCECRCRDQSRDRSLCHGKGFLECGICRCDTGYIGKNCECQTQGRSSQELEGSCRKDNNSIICSGLGDCVCGQCLCHTSDVPGKLIYGQYCECDTINCERYNGQVCGGPGRGLCFCGKCRCHPGFEGSACQCERTTEGCLNPRRVECSGRGRCRCNVCECHSGYQLPLCQECPGCPSPCGKYISCAECLKFEKGPFGKNCSAACPGLQLSNNPVKGRTCKERDSEGCWVAYTLEQQDGMDRYLIYVDESRECVAGPNIAAIVGGTVAGIVLIGILLLVIWKALIHLSDLREYRRFEKEKLKSQWNNDNPLFKSATTTVMNPKFAES

SEQ ID NO: 85 - DNA, 2307 bp, Homo sapiens
  source 1..2307: mol_type = genomic DNA
atgctgggcctgcgccccccacttctcgccctggtggggctgctctccctcgggtgcgtcctctctcaggagtgcacgaagttcaaggtcagcagctgccgggaatgcatcgagtcggggcccggctgcacctggtgccagaagctgaacttcacagggccgggggatcctgactccattcgctgcgacacccggccacagctgctcatgaggggctgtgcggctgacgacatcatggaccccacaagcctcgctgaaacccaggaagaccacaatgggggccagaagcagctgtccccacaaaaagtgacgctttacctgcgaccaggccaggcagcagcgttcaacgtgaccttccggcgggccaagggctaccccatcgacctgtactatctgatggacctctcctactccatgcttgatgacctcaggaatgtcaagaagctaggtggcgacctgctccgggccctcaacgagatcaccgagtccggccgcattggcttcgggtccttcgtggacaagaccgtgctgccgttcgtgaacacgcaccctgataagctgcgaaacccatgccccaacaaggagaaagagtgccagcccccgtttgccttcaggcacgtgctgaagctgaccaacaactccaaccagtttcagaccgaggtcgggaagcagctgatttccggaaacctggatgcacccgagggtgggctggacgccatgatgcaggtcgccgcctgcccggaggaaatcggctggcgcaacgtcacgcggctgctggtgtttgccactgatgacggcttccatttcgcgggcgacgggaagctgggcgccatcctgacccccaacgacggccgctgtcacctggaggacaacttgtacaagaggagcaacgaattcgactacccatcggtgggccagctggcgcacaagctggctgaaaacaacatccagcccatcttcgcggtgaccagtaggatggtgaagacctacgagaaactcaccgagatcatccccaagtcagccgtgggggagctgtctgaggactccagcaatgtggtccatctcattaagaatgcttacaataaactctcctccagggtattcctggatcacaacgccctccccgacaccctgaaagtcacctacgactccttctgcagcaatggagtgacgcacaggaaccagcccagaggtgactgtgatggcgtgcagatcaatgtcccgatcaccttccaggtgaaggtcacggccacagagtgcatccaggagcagtcgtttgtcatccgggcgctgggcttcacggacatagtgaccgtgcaggtccttccccagtgtgagtgccggtgccgggaccagagcagagaccgcagcctctgccatggcaagggcttcttggagtgcggcatctgcaggtgtgacactggctacattgggaaaaactgtgagtgccagacacagggccggagcagccaggagctggaaggaagctgccggaaggacaacaactccatcatctgctcagggctgggggactgtgtctgcgggcagtgcctgtgccacaccagcgacgtccccggcaagctgatatacgggcagtactgcgagtgtgacaccatcaactgtgagcgctacaacggccaggtctgcggcggcccggggagggggctctgcttctgcgggaagtgccgctgccacccgggctttgagggctcagcgtgccagtgcgagaggaccactgagggctgcctgaacccgcggcgtgttgagtgtagtggtcgtggccggtgccgctgcaacgtatgcgagtgccattcaggctaccagctgcctctgtgccaggagtgccccggctgcccctcaccctgtggcaagtacatctcctgcgccgagtgcctgaagttcgaaaagggcccctttgggaagaactgcagcgcggcgtgtccgggcctgcagctgtcgaacaaccccgtgaagggcaggacctgcaaggagagggactcagagggctgctgggtggcctacacgctggagcagcaggacgggatggaccgctacctcatctatgtggatgagagccgagagtgtgtggcaggccccaacatcgccgccatcgtcgggggcaccgtggcaggcatcgtgctgatcggcattctcctgctggtcatctggaaggctctgatccacctgagcgacctccgggagtacaggcgctttgagaaggagaagctcaagtcccagtggaacaatgataatccccttttcaagagcgccaccacgacggtcatgaaccccaagtttgctgagagt

SEQ ID NO: 86 - protein, 769 aa, Homo sapiens
MLGLRPPLLALVGLLSLGCVLSQECTKFKVSSCRECIESGPGCTWCQKLNFTGPGDPDSIRCDTRPQLLMRGCAADDIMDPTSLAETQEDHNGGQKQLSPQKVTLYLRPGQAAAFNVTFRRAKGYPIDLYYLMDLSYSMLDDLRNVKKLGGDLLRALNEITESGRIGFGSFVDKTVLPFVNTHPDKLRNPCPNKEKECQPPFAFRHVLKLTNNSNQFQTEVGKQLISGNLDAPEGGLDAMMQVAACPEEIGWRNVTRLLVFATDDGFHFAGDGKLGAILTPNDGRCHLEDNLYKRSNEFDYPSVGQLAHKLAENNIQPIFAVTSRMVKTYEKLTEIIPKSAVGELSEDSSNVVHLIKNAYNKLSSRVFLDHNALPDTLKVTYDSFCSNGVTHRNQPRGDCDGVQINVPITFQVKVTATECIQEQSFVIRALGFTDIVTVQVLPQCECRCRDQSRDRSLCHGKGFLECGICRCDTGYIGKNCECQTQGRSSQELEGSCRKDNNSIICSGLGDCVCGQCLCHTSDVPGKLIYGQYCECDTINCERYNGQVCGGPGRGLCFCGKCRCHPGFEGSACQCERTTEGCLNPRRVECSGRGRCRCNVCECHSGYQLPLCQECPGCPSPCGKYISCAECLKFEKGPFGKNCSAACPGLQLSNNPVKGRTCKERDSEGCWVAYTLEQQDGMDRYLIYVDESRECVAGPNIAAIVGGTVAGIVLIGILLLVIWKALIHLSDLREYRRFEKEKLKSQWNNDNPLFKSATTTVMNPKFAES

SEQ ID NO: 87 - DNA, 3462 bp, Synthetic construct
  source 1..3462: mol_type = other DNA
atgcagtatctaaatataaaagaggactgcaatgccatggctttctgtgctaaaatgaggagctccaagaagactgaggtgaacctggaggcccctgagccaggggtggaagtgatcttctatctgtcggacagggagcccctccggctgggcagtggagagtacacagcagaggaactgtgcatcagggctgcacaggcatgccgtatctctcctctttgtcacaacctctttgccctgtatgacgagaacaccaagctctggtatgctccaaatcgcaccatcaccgttgatgacaagatgtccctccggctccactaccggatgaggttctatttcaccaattggcatggaaccaacgacaatgagcagtcagtgtggcgtcattctccaaagaagcagaaaaatggctacgagaaaaaaaagattccagatgcaacccctctccttgatgccagctcactggagtatctgtttgctcagggacagtatgatttggtgaaatgcctggctcctattcgagaccccaagaccgagcaggatggacatgatattgagaacgagtgtctagggatggctgtcctggccatctcacactatgccatgatgaagaagatgcagttgccagaactgcccaaggacatcagctacaagcgatatattccagaaacattgaataagtccatcagacagaggaaccttctcaccaggatgcggataaataatgttttcaaggatttcctaaaggaatttaacaacaagaccatttgtgacagcagcgtgtccacgcatgacctgaaggtgaaatacttggctaccttggaaactttgacaaaacattacggtgctgaaatatttgagacttccatgttactgatttcatcagaaaatgagatgaattggtttcattcgaatgacggtggaaacgttctctactacgaagtgatggtgactgggaatcttggaatccagtggaggcataaaccaaatgttgtttctgttgaaaaggaaaaaaataaactgaagcggaaaaaactggaaaataaacacaagaaggatgaggagaaaaacaagatccgggaagagtggaacaatttttcttacttccctgaaatcactcacattgtaataaaggagtctgtggtcagcattaacaagcaggacaacaagaaaatggaactgaagctctcttcccacgaggaggccttgtcctttgtgtccctggtagatggctacttccggctcacagcagatgcccatcattacctctgcaccgacgtggcccccccgttgatcgtccacaacatacagaatggctgtcatggtccaatctgtacagaatacgccatcaataaattgcggcaagaaggaagcgaggaggggatgtacgtgctgaggtggagctgcaccgactttgacaacatcctcatgaccgtcacctgctttgagaagtctgagcaggtgcagggtgcccagaagcagttcaagaactttcagatcgaggtgcagaagggccgctacagtctgcacggttcggaccgcagcttccccagcttgggagacctcatgagccacctcaagaagcagatcctgcgcacggataacatcagcttcatgctaaaacgctgctgccagcccaagccccgagaaatctccaacctgctggtggctactaagaaagcccaggagtggcagcccgtctaccccatgagccagctgagtttcgatcggatcctcaagaaggatctggtgcagggcgagcaccttgggagaggcacgagaacacacatctattctgggaccctgatggattacaaggatgacgaaggaacttctgaagagaagaagataaaagtgatcctcaaagtcttagaccccagccacagggatatttccctggccttcttcgaggcagccagcatgatgagacaggtctcccacaaacacatcgtgtacctctatggcgtctgtgtccgcgacgtggagaatatcatggtggaagagtttgtggaagggggtcctctggatctcttcatgcaccggaaaagcgatgtccttaccacaccatggaaattcaaagttgccaaacagctggccagtgccctgagctacttggaggataaagacctggtccatggaaatgtgtgtactaaaaacctcctcctggcccgtgagggcatcgacagtgagtgtggcccattcatcaagctcagtgaccccggcatccccattacggtgctgtctaggcaagaatgcattgaacgaatcccatggattgctcctgagtgtgttgaggactccaagaacctgagtgtggctgctgacaagtggagctttggaaccacgctctgggaaatctgctacaatggcgagatccccttgaaagacaagacgctgattgagaaagagagattctatgaaagccggtgcaggccagtgacaccatcatgtaaggagctggctgacctcatgacccgctgcatgaactatgaccccaatcagaggcctttcttccgagccatcatgagagacattaataagcttgaagagcagaatccagatattgtttcagaaaaaaaaccagcaactgaagtggaccccacacattttgaaaagcgcttcctaaagaggatccgtgacttgggagagggccactttgggaaggttgagctctgcaggtatgaccccgaaggggacaatacaggggagcaggtggctgttaaatctctgaagcctgagagtggaggtaaccacatagctgatctgaaaaaggaaatcgagatcttaaggaacctctatcatgagaacattgtgaagtacaaaggaatctgcacagaagacggaggaaatggtattaagctcatcatggaatttctgccttcgggaagccttaaggaatatcttccaaagaataagaacaaaataaacctcaaacagcagctaaaatatgccgttcagatttgtaaggggatggactatttgggttctcggcaatacgttcaccgggacttggcagcaagaaatgtccttgttgagagtgaacaccaagtgaaaattggagacttcggtttaaccaaagcaattgaaaccgataaggagtattacaccgtcaaggatgaccgggacagccctgtgttttggtatgctccagaatgtttaatgcaatctaaattttatattgcctctgacgtctggtcttttggagtcactctgcatgagctgctgacttactgtgattcagattctagtcccatggctttgttcctgaaaatgataggcccaacccatgcccagatgacagtcacaagacttgtgaatacgttaaaagaaggaaaacgcctgccgtgcccacctaactgtccagatgaggtttatcaacttatgaggaaatgctgggaattccaaccatccaatcggacaagctttcagaaccttattgaaggatttgaagcacttttaaaa

SEQ ID NO: 88 - protein, 1154 aa, Synthetic construct
MQYLNIKEDCNAMAFCAKMRSSKKTEVNLEAPEPGVEVIFYLSDREPLRLGSGEYTAEELCIRAAQACRISPLCHNLFALYDENTKLWYAPNRTITVDDKMSLRLHYRMRFYFTNWHGTNDNEQSVWRHSPKKQKNGYEKKKIPDATPLLDASSLEYLFAQGQYDLVKCLAPIRDPKTEQDGHDIENECLGMAVLAISHYAMMKKMQLPELPKDISYKRYIPETLNKSIRQRNLLTRMRINNVFKDFLKEFNNKTICDSSVSTHDLKVKYLATLETLTKHYGAEIFETSMLLISSENEMNWFHSNDGGNVLYYEVMVTGNLGIQWRHKPNVVSVEKEKNKLKRKKLENKHKKDEEKNKIREEWNNFSYFPEITHIVIKESVVSINKQDNKKMELKLSSHEEALSFVSLVDGYFRLTADAHHYLCTDVAPPLIVHNIQNGCHGPICTEYAINKLRQEGSEEGMYVLRWSCTDFDNILMTVTCFEKSEQVQGAQKQFKNFQIEVQKGRYSLHGSDRSFPSLGDLMSHLKKQILRTDNISFMLKRCCQPKPREISNLLVATKKAQEWQPVYPMSQLSFDRILKKDLVQGEHLGRGTRTHIYSGTLMDYKDDEGTSEEKKIKVILKVLDPSHRDISLAFFEAASMMRQVSHKHIVYLYGVCVRDVENIMVEEFVEGGPLDLFMHRKSDVLTTPWKFKVAKQLASALSYLEDKDLVHGNVCTKNLLLAREGIDSECGPFIKLSDPGIPITVLSRQECIERIPWIAPECVEDSKNLSVAADKWSFGTTLWEICYNGEIPLKDKTLIEKERFYESRCRPVTPSCKELADLMTRCMNYDPNQRPFFRAIMRDINKLEEQNPDIVSEKKPATEVDPTHFEKRFLKRIRDLGEGHFGKVELCRYDPEGDNTGEQVAVKSLKPESGGNHIADLKKEIEILRNLYHENIVKYKGICTEDGGNGIKLIMEFLPSGSLKEYLPKNKNKINLKQQLKYAVQICKGMDYLGSRQYVHRDLAARNVLVESEHQVKIGDFGLTKAIETDKEYYTVKDDRDSPVFWYAPECLMQSKFYIASDVWSFGVTLHELLTYCDSDSSPMALFLKMIGPTHAQMTVTRLVNTLKEGKRLPCPPNCPDEVYQLMRKCWEFQPSNRTSFQNLIEGFEALLK

SEQ ID NO: 89 - DNA, 3462 bp, Homo sapiens
  source 1..3462: mol_type = genomic DNA
atgcagtatctaaatataaaagaggactgcaatgccatggctttctgtgctaaaatgaggagctccaagaagactgaggtgaacctggaggcccctgagccaggggtggaagtgatcttctatctgtcggacagggagcccctccggctgggcagtggagagtacacagcagaggaactgtgcatcagggctgcacaggcatgccgtatctctcctctttgtcacaacctctttgccctgtatgacgagaacaccaagctctggtatgctccaaatcgcaccatcaccgttgatgacaagatgtccctccggctccactaccggatgaggttctatttcaccaattggcatggaaccaacgacaatgagcagtcagtgtggcgtcattctccaaagaagcagaaaaatggctacgagaaaaaaaagattccagatgcaacccctctccttgatgccagctcactggagtatctgtttgctcagggacagtatgatttggtgaaatgcctggctcctattcgagaccccaagaccgagcaggatggacatgatattgagaacgagtgtctagggatggctgtcctggccatctcacactatgccatgatgaagaagatgcagttgccagaactgcccaaggacatcagctacaagcgatatattccagaaacattgaataagtccatcagacagaggaaccttctcaccaggatgcggataaataatgttttcaaggatttcctaaaggaatttaacaacaagaccatttgtgacagcagcgtgtccacgcatgacctgaaggtgaaatacttggctaccttggaaactttgacaaaacattacggtgctgaaatatttgagacttccatgttactgatttcatcagaaaatgagatgaattggtttcattcgaatgacggtggaaacgttctctactacgaagtgatggtgactgggaatcttggaatccagtggaggcataaaccaaatgttgtttctgttgaaaaggaaaaaaataaactgaagcggaaaaaactggaaaataaacacaagaaggatgaggagaaaaacaagatccgggaagagtggaacaatttttcttacttccctgaaatcactcacattgtaataaaggagtctgtggtcagcattaacaagcaggacaacaagaaaatggaactgaagctctcttcccacgaggaggccttgtcctttgtgtccctggtagatggctacttccggctcacagcagatgcccatcattacctctgcaccgacgtggcccccccgttgatcgtccacaacatacagaatggctgtcatggtccaatctgtacagaatacgccatcaataaattgcggcaagaaggaagcgaggaggggatgtacgtgctgaggtggagctgcaccgactttgacaacatcctcatgaccgtcacctgctttgagaagtctgagcaggtgcagggtgcccagaagcagttcaagaactttcagatcgaggtgcagaagggccgctacagtctgcacggttcggaccgcagcttccccagcttgggagacctcatgagccacctcaagaagcagatcctgcgcacggataacatcagcttcatgctaaaacgctgctgccagcccaagccccgagaaatctccaacctgctggtggctactaagaaagcccaggagtggcagcccgtctaccccatgagccagctgagtttcgatcggatcctcaagaaggatctggtgcagggcgagcaccttgggagaggcacgagaacacacatctattctgggaccctgatggattacaaggatgacgaaggaacttctgaagagaagaagataaaagtgatcctcaaagtcttagaccccagccacagggatatttccctggccttcttcgaggcagccagcatgatgagacaggtctcccacaaacacatcgtgtacctctatggcgtctgtgtccgcgacgtggagaatatcatggtggaagagtttgtggaagggggtcctctggatctcttcatgcaccggaaaagcgatgtccttaccacaccatggaaattcaaagttgccaaacagctggccagtgccctgagctacttggaggataaagacctggtccatggaaatgtgtgtactaaaaacctcctcctggcccgtgagggcatcgacagtgagtgtggcccattcatcaagctcagtgaccccggcatccccattacggtgctgtctaggcaagaatgcattgaacgaatcccatggattgctcctgagtgtgttgaggactccaagaacctgagtgtggctgctgacaagtggagctttggaaccacgctctgggaaatctgctacaatggcgagatccccttgaaagacaagacgctgattgagaaagagagattctatgaaagccggtgcaggccagtgacaccatcatgtaaggagctggctgacctcatgacccgctgcatgaactatgaccccaatcagaggcctttcttccgagccatcatgagagacattaataagcttgaagagcagaatccagatattgtttcagaaaaaaaaccagcaactgaagtggaccccacacattttgaaaagcgcttcctaaagaggatccgtgacttgggagagggccactttgggaaggttgagctctgcaggtatgaccccgaaggggacaatacaggggagcaggtggctgttaaatctctgaagcctgagagtggaggtaaccacatagctgatctgaaaaaggaaatcgagatcttaaggaacctctatcatgagaacattgtgaagtacaaaggaatctgcacagaagacggaggaaatggtattaagctcatcatggaatttctgccttcgggaagccttaaggaatatcttccaaagaataagaacaaaataaacctcaaacagcagctaaaatatgccgttcagatttgtaaggggatggactatttgggttctcggcaatacgttcaccgggacttggcagcaagaaatgtccttgttgagagtgaacaccaagtgaaaattggagacttcggtttaaccaaagcaattgaaaccgataaggagtattacaccgtcaaggatgaccgggacagccctgtgttttggtatgctccagaatgtttaatgcaatctaaattttatattgcctctgacgtctggtcttttggagtcactctgcatgagctgctgacttactgtgattcagattctagtcccatggctttgttcctgaaaatgataggcccaacccatggccagatgacagtcacaagacttgtgaatacgttaaaagaaggaaaacgcctgccgtgcccacctaactgtccagatgaggtttatcaacttatgaggaaatgctgggaattccaaccatccaatcggacaagctttcagaaccttattgaaggatttgaagcacttttaaaa

SEQ ID NO: 90 - protein, 1154 aa, Homo sapiens
MQYLNIKEDCNAMAFCAKMRSSKKTEVNLEAPEPGVEVIFYLSDREPLRLGSGEYTAEELCIRAAQACRISPLCHNLFALYDENTKLWYAPNRTITVDDKMSLRLHYRMRFYFTNWHGTNDNEQSVWRHSPKKQKNGYEKKKIPDATPLLDASSLEYLFAQGQYDLVKCLAPIRDPKTEQDGHDIENECLGMAVLAISHYAMMKKMQLPELPKDISYKRYIPETLNKSIRQRNLLTRMRINNVFKDFLKEFNNKTICDSSVSTHDLKVKYLATLETLTKHYGAEIFETSMLLISSENEMNWFHSNDGGNVLYYEVMVTGNLGIQWRHKPNVVSVEKEKNKLKRKKLENKHKKDEEKNKIREEWNNFSYFPEITHIVIKESVVSINKQDNKKMELKLSSHEEALSFVSLVDGYFRLTADAHHYLCTDVAPPLIVHNIQNGCHGPICTEYAINKLRQEGSEEGMYVLRWSCTDFDNILMTVTCFEKSEQVQGAQKQFKNFQIEVQKGRYSLHGSDRSFPSLGDLMSHLKKQILRTDNISFMLKRCCQPKPREISNLLVATKKAQEWQPVYPMSQLSFDRILKKDLVQGEHLGRGTRTHIYSGTLMDYKDDEGTSEEKKIKVILKVLDPSHRDISLAFFEAASMMRQVSHKHIVYLYGVCVRDVENIMVEEFVEGGPLDLFMHRKSDVLTTPWKFKVAKQLASALSYLEDKDLVHGNVCTKNLLLAREGIDSECGPFIKLSDPGIPITVLSRQECIERIPWIAPECVEDSKNLSVAADKWSFGTTLWEICYNGEIPLKDKTLIEKERFYESRCRPVTPSCKELADLMTRCMNYDPNQRPFFRAIMRDINKLEEQNPDIVSEKKPATEVDPTHFEKRFLKRIRDLGEGHFGKVELCRYDPEGDNTGEQVAVKSLKPESGGNHIADLKKEIEILRNLYHENIVKYKGICTEDGGNGIKLIMEFLPSGSLKEYLPKNKNKINLKQQLKYAVQICKGMDYLGSRQYVHRDLAARNVLVESEHQVKIGDFGLTKAIETDKEYYTVKDDRDSPVFWYAPECLMQSKFYIASDVWSFGVTLHELLTYCDSDSSPMALFLKMIGPTHGQMTVTRLVNTLKEGKRLPCPPNCPDEVYQLMRKCWEFQPSNRTSFQNLIEGFEALLK

SEQ ID NO: 91 - DNA, 3375 bp, Synthetic construct
  source 1..3375: mol_type = other DNA
atggcacctccaagtgaagagacgcccctgatccctcagcgttcatgcagcctcttgtccacggaggctggtgccctgcatgtgctgctgcccgctcggggccccgggcccccccagcgcctatctttctcctttggggaccacttggctgaggacctgtgcgtgcaggctgccaaggccagcggcatcctgcctgtgtaccactccctctttgctctggccacggaggacctgtcctgctggttccccccgagccacatcttctccgtggaggatgccagcacccaagtcctgctgtacaggattcgcttttacttccccaattggtttgggctggagaagtgccaccgcttcgggctacgcaaggatttggccagtgctatccttgacctgccagtcctggagcacctctttgcccagcaccgcagtgacctggtgagtgggcgcctccccgtgggcctcagtctcaaggagcagggtgagtgtctcagcctggccgtgttggacctggcccggatggcgcgagagcaggcccagcggccgggagagctgctgaagactgtcagctacaaggcctgcctacccccaagcctgcgcgacctgatccagggcctgagcttcgtgacgcggaggcgtattcggaggacggtgcgcagagccctgcgccgcgtggccgcctgccaggcagaccggcactcgctcatggccaagtacatcatggacctggagcggctggatccagccggggccgccgagaccttccacgtgggcctccctggggcccttggtggccacgacgggctggggctgctccgcgtggctggtgacggcggcatcgcctggacccagggagaacaggaggtcctccagcccttctgcgactttccagaaatcgtagacattagcatcaagcaggccccgcgcgttggcccggccggagagcaccgcctggtcactgttaccaggacagacaaccagattttagaggccgagttcccagggctgcccgaggctctgtcgttcgtggcgctcgtggacggctacttccggctgaccacggactcccagcacttcttctgcaaggaggtggcaccgccgaggctgctggaggaagtggccgagcagtgccacggccccatcactctggactttgccatcaacaagctcaagactgggggctcacgtcctggctcctatgttctccgccgcagcccccaggactttgacagcttcctcctcactgtctgtgtccagaacccccttggtcctgattataagggctgcctcatccggcgcagccccacaggaaccttccttctggttggcctcagccgaccccacagcagtcttcgagagctcctggcaacctgctgggatggggggctgcacgtagatggggtggcagtgaccctcacttcctgctgtatccccagacccaaagaaaagtccaacctgatcgtggtccagagaggtcacagcccacccacatcatccttggttcagccccaatcccaataccagctgagtcagatgacatttcacaagatccctgctgacagcctggagtggcatgagaacctgggccatgggtccttcaccaagatttaccggggctgtcgccatgaggtggtggatggggaggcccgaaagacagaggtgctgctgaaggtcatggatgccaagcacaagaactgcatggagtcattcctggaagcagtgagcttgatgagccaagtgtcgtaccggcatctcgtgctgctccacggcgtgtgcatggctggagacagcaccatggtgcaggaatttgtacacctgggggccatagacatgtatctgcgaaaacgtggccacctggtgccagccagctggaagctgcaggtggtcaaacagctggcctacgccctcaactatctggaggacaaaggcctgccccatggcaatgtctctgcccggaaggtgctcctggctcgggagggggctgatgggagcccgcccttcatcaagctgagtgaccctggggtcagccccgctgtgttaagcctggagatgctcaccgacaggatcccctgggtggcccccgagtgtctccgggaggcgcagacacttagcttggaagctgacaagtggggcttcggcgccacggtctgggaagtgtttagtggcgtcaccatgcccatcagtgccctggatcctgctaagaaactccaattttatgaggaccggcagcagctgccggcccccaagtggacagagctggccctgctgattcaacagtgcatggcctatgagccggtccagaggccctccttccgagccgtcattcgtgacctcaatagcctcatctcttcagactatgagctcctctcagaccccacacctggtgccctggcacctcgtgatgggctgtggaatggtgcccagctctatgcctgccaagaccccacgatcttcgaggagagacacctcaagtacatctcacagctgggcaagggcaactttggcagcgtggagctgtgccgctatgacccgctaggcgacaatacaggtgccctggtggccgtgaaacagctgcagcacagcgggccagaccagcagagggactttcagcgggagattcagatcctcaaagcactgcacagtgatttcattgtcaagtatcgtggtgtcagctatggcccgggccgccagagcctgcggctggtcatggagtacctgcccagcggctgcttgcgcgacttcctgcagcggcaccgcgcgcgcctcgatgccagccgcctccttctctattcctcgcagatctgcaagggcatggagtacctgggctcccgccgctgcgtgcaccgcgacctggccgcccgaaacatcctcgtggagagcgaggcacacgtcaagatcgctgacttcggcctagctaagctgctgccgcttgacaaagactactacgtggtccgcgagccaggccagagccccattttctggtatgcccccgaatccctctcggacaacatcttctctcgccagtcagacgtctggagcttcggggtcgtcctgtacgagctcttcacctactgcgacaaaagctgcagcccctcggccgagttcctgcggatgatgggatgtgagcgggatgtccccgccctctgccgcctcttggaactgctggaggagggccagaggctgccggcgcctcctgcctgccctgctgaggttcacgagctcatgaagctgtgctgggcccctagcccacaggaccggccatcattcagcgccctgggcccccagctggacatgctgtggagcggaagccgggggtgtgagactcatgccttcactgctcacccagagggcaaacaccactccctgtccttttcattg

SEQ ID NO: 92 - protein, 1125 aa, Synthetic construct
MAPPSEETPLIPQRSCSLLSTEAGALHVLLPARGPGPPQRLSFSFGDHLAEDLCVQAAKASGILPVYHSLFALATEDLSCWFPPSHIFSVEDASTQVLLYRIRFYFPNWFGLEKCHRFGLRKDLASAILDLPVLEHLFAQHRSDLVSGRLPVGLSLKEQGECLSLAVLDLARMAREQAQRPGELLKTVSYKACLPPSLRDLIQGLSFVTRRRIRRTVRRALRRVAACQADRHSLMAKYIMDLERLDPAGAAETFHVGLPGALGGHDGLGLLRVAGDGGIAWTQGEQEVLQPFCDFPEIVDISIKQAPRVGPAGEHRLVTVTRTDNQILEAEFPGLPEALSFVALVDGYFRLTTDSQHFFCKEVAPPRLLEEVAEQCHGPITLDFAINKLKTGGSRPGSYVLRRSPQDFDSFLLTVCVQNPLGPDYKGCLIRRSPTGTFLLVGLSRPHSSLRELLATCWDGGLHVDGVAVTLTSCCIPRPKEKSNLIVVQRGHSPPTSSLVQPQSQYQLSQMTFHKIPADSLEWHENLGHGSFTKIYRGCRHEVVDGEARKTEVLLKVMDAKHKNCMESFLEAVSLMSQVSYRHLVLLHGVCMAGDSTMVQEFVHLGAIDMYLRKRGHLVPASWKLQVVKQLAYALNYLEDKGLPHGNVSARKVLLAREGADGSPPFIKLSDPGVSPAVLSLEMLTDRIPWVAPECLREAQTLSLEADKWGFGATVWEVFSGVTMPISALDPAKKLQFYEDRQQLPAPKWTELALLIQQCMAYEPVQRPSFRAVIRDLNSLISSDYELLSDPTPGALAPRDGLWNGAQLYACQDPTIFEERHLKYISQLGKGNFGSVELCRYDPLGDNTGALVAVKQLQHSGPDQQRDFQREIQILKALHSDFIVKYRGVSYGPGRQSLRLVMEYLPSGCLRDFLQRHRARLDASRLLLYSSQICKGMEYLGSRRCVHRDLAARNILVESEAHVKIADFGLAKLLPLDKDYYVVREPGQSPIFWYAPESLSDNIFSRQSDVWSFGVVLYELFTYCDKSCSPSAEFLRMMGCERDVPALCRLLELLEEGQRLPAPPACPAEVHELMKLCWAPSPQDRPSFSALGPQLDMLWSGSRGCETHAFTAHPEGKHHSLSFSL

SEQ ID NO: 93 - DNA, 3375 bp, Homo sapiens
  source 1..3375: mol_type = genomic DNA
atggcacctccaagtgaagagacgcccctgatccctcagcgttcatgcagcctcttgtccacggaggctggtgccctgcatgtgctgctgcccgctcggggccccgggcccccccagcgcctatctttctcctttggggaccacttggctgaggacctgtgcgtgcaggctgccaaggccagcggcatcctgcctgtgtaccactccctctttgctctggccacggaggacctgtcctgctggttccccccgagccacatcttctccgtggaggatgccagcacccaagtcctgctgtacaggattcgcttttacttccccaattggtttgggctggagaagtgccaccgcttcgggctacgcaaggatttggccagtgctatccttgacctgccagtcctggagcacctctttgcccagcaccgcagtgacctggtgagtgggcgcctccccgtgggcctcagtctcaaggagcagggtgagtgtctcagcctggccgtgttggacctggcccggatggcgcgagagcaggcccagcggccgggagagctgctgaagactgtcagctacaaggcctgcctacccccaagcctgcgcgacctgatccagggcctgagcttcgtgacgcggaggcgtattcggaggacggtgcgcagagccctgcgccgcgtggccgcctgccaggcagaccggcactcgctcatggccaagtacatcatggacctggagcggctggatccagccggggccgccgagaccttccacgtgggcctccctggggcccttggtggccacgacgggctggggctgctccgcgtggctggtgacggcggcatcgcctggacccagggagaacaggaggtcctccagcccttctgcgactttccagaaatcgtagacattagcatcaagcaggccccgcgcgttggcccggccggagagcaccgcctggtcactgttaccaggacagacaaccagattttagaggccgagttcccagggctgcccgaggctctgtcgttcgtggcgctcgtggacggctacttccggctgaccacggactcccagcacttcttctgcaaggaggtggcaccgccgaggctgctggaggaagtggccgagcagtgccacggccccatcactctggactttgccatcaacaagctcaagactgggggctcacgtcctggctcctatgttctccgccgcagcccccaggactttgacagcttcctcctcactgtctgtgtccagaacccccttggtcctgattataagggctgcctcatccggcgcagccccacaggaaccttccttctggttggcctcagccgaccccacagcagtcttcgagagctcctggcaacctgctgggatggggggctgcacgtagatggggtggcagtgaccctcacttcctgctgtatccccagacccaaagaaaagtccaacctgatcgtggtccagagaggtcacagcccacccacatcatccttggttcagccccaatcccaataccagctgagtcagatgacatttcacaagatccctgctgacagcctggagtggcatgagaacctgggccatgggtccttcaccaagatttaccggggctgtcgccatgaggtggtggatggggaggcccgaaagacagaggtgctgctgaaggtcatggatgccaagcacaagaactgcatggagtcattcctggaagcagcgagcttgatgagccaagtgtcgtaccggcatctcgtgctgctccacggcgtgtgcatggctggagacagcaccatggtgcaggaatttgtacacctgggggccatagacatgtatctgcgaaaacgtggccacctggtgccagccagctggaagctgcaggtggtcaaacagctggcctacgccctcaactatctggaggacaaaggcctgccccatggcaatgtctctgcccggaaggtgctcctggctcgggagggggctgatgggagcccgcccttcatcaagctgagtgaccctggggtcagccccgctgtgttaagcctggagatgctcaccgacaggatcccctgggtggcccccgagtgtctccgggaggcgcagacacttagcttggaagctgacaagtggggcttcggcgccacggtctgggaagtgtttagtggcgtcaccatgcccatcagtgccctggatcctgctaagaaactccaattttatgaggaccggcagcagctgccggcccccaagtggacagagctggccctgctgattcaacagtgcatggcctatgagccggtccagaggccctccttccgagccgtcattcgtgacctcaatagcctcatctcttcagactatgagctcctctcagaccccacacctggtgccctggcacctcgtgatgggctgtggaatggtgcccagctctatgcctgccaagaccccacgatcttcgaggagagacacctcaagtacatctcacagctgggcaagggcaactttggcagcgtggagctgtgccgctatgacccgctaggcgacaatacaggtgccctggtggccgtgaaacagctgcagcacagcgggccagaccagcagagggactttcagcgggagattcagatcctcaaagcactgcacagtgatttcattgtcaagtatcgtggtgtcagctatggcccgggccgccagagcctgcggctggtcatggagtacctgcccagcggctgcttgcgcgacttcctgcagcggcaccgcgcgcgcctcgatgccagccgcctccttctctattcctcgcagatctgcaagggcatggagtacctgggctcccgccgctgcgtgcaccgcgacctggccgcccgaaacatcctcgtggagagcgaggcacacgtcaagatcgctgacttcggcctagctaagctgctgccgcttgacaaagactactacgtggtccgcgagccaggccagagccccattttctggtatgcccccgaatccctctcggacaacatcttctctcgccagtcagacgtctggagcttcggggtcgtcctgtacgagctcttcacctactgcgacaaaagctgcagcccctcggccgagttcctgcggatgatgggatgtgagcgggatgtccccgccctctgccgcctcttggaactgctggaggagggccagaggctgccggcgcctcctgcctgccctgctgaggttcacgagctcatgaagctgtgctgggcccctagcccacaggaccggccatcattcagcgccctgggcccccagctggacatgctgtggagcggaagccgggggtgtgagactcatgccttcactgctcacccagagggcaaacaccactccctgtccttttcattg

SEQ ID NO: 94 - protein, 1125 aa, Homo sapiens
MAPPSEETPLIPQRSCSLLSTEAGALHVLLPARGPGPPQRLSFSFGDHLAEDLCVQAAKASGILPVYHSLFALATEDLSCWFPPSHIFSVEDASTQVLLYRIRFYFPNWFGLEKCHRFGLRKDLASAILDLPVLEHLFAQHRSDLVSGRLPVGLSLKEQGECLSLAVLDLARMAREQAQRPGELLKTVSYKACLPPSLRDLIQGLSFVTRRRIRRTVRRALRRVAACQADRHSLMAKYIMDLERLDPAGAAETFHVGLPGALGGHDGLGLLRVAGDGGIAWTQGEQEVLQPFCDFPEIVDISIKQAPRVGPAGEHRLVTVTRTDNQILEAEFPGLPEALSFVALVDGYFRLTTDSQHFFCKEVAPPRLLEEVAEQCHGPITLDFAINKLKTGGSRPGSYVLRRSPQDFDSFLLTVCVQNPLGPDYKGCLIRRSPTGTFLLVGLSRPHSSLRELLATCWDGGLHVDGVAVTLTSCCIPRPKEKSNLIVVQRGHSPPTSSLVQPQSQYQLSQMTFHKIPADSLEWHENLGHGSFTKIYRGCRHEVVDGEARKTEVLLKVMDAKHKNCMESFLEAASLMSQVSYRHLVLLHGVCMAGDSTMVQEFVHLGAIDMYLRKRGHLVPASWKLQVVKQLAYALNYLEDKGLPHGNVSARKVLLAREGADGSPPFIKLSDPGVSPAVLSLEMLTDRIPWVAPECLREAQTLSLEADKWGFGATVWEVFSGVTMPISALDPAKKLQFYEDRQQLPAPKWTELALLIQQCMAYEPVQRPSFRAVIRDLNSLISSDYELLSDPTPGALAPRDGLWNGAQLYACQDPTIFEERHLKYISQLGKGNFGSVELCRYDPLGDNTGALVAVKQLQHSGPDQQRDFQREIQILKALHSDFIVKYRGVSYGPGRQSLRLVMEYLPSGCLRDFLQRHRARLDASRLLLYSSQICKGMEYLGSRRCVHRDLAARNILVESEAHVKIADFGLAKLLPLDKDYYVVREPGQSPIFWYAPESLSDNIFSRQSDVWSFGVVLYELFTYCDKSCSPSAEFLRMMGCERDVPALCRLLELLEEGQRLPAPPACPAEVHELMKLCWAPSPQDRPSFSALGPQLDMLWSGSRGCETHAFTAHPEGKHHSLSFSL

SEQ ID NO: 95 - DNA, 1041 bp, Synthetic construct
  source 1..1041: mol_type = other DNA
atgtgcactaaaatggaacagcccttctaccacgacgactcatacacagctacgggatacggccgggcccctggtggcctctctctacacgactacaaactcctgaaaccgagcctggcggtcaacctggccgacccctaccggagtctcaaagcgcctggggctcgcggacccggcccagagggcggcggtggcggcagctacttttctggtcagggctcggacaccggcgcgtctctcaagctcgcctcttcggagctggaacgcctgattgtccccaacagcaacggcgtgatcacgacgacgcctacacccccgggacagtacttttacccccgcgggggtggcagcggtggaggtgcagggggcgcagggggcggcgtcaccgaggagcaggagggcttcgccgacggctttgtcaaagccctggacgatctgcacaagatgaaccacgtgacaccccccaacgtgtccctgggcgctaccggggggcccccggctgggcccgggggcgtctacgccggcccggagccacctcccgtttacaccaacctcagcagctactccccagcctctgcgtcctcgggaggcgccggggctgccgtcgggaccgggagctcgtacccgacgaccaccatcagctacctcccacacgcgccgcccttcgccggtggccacccggcgcagctgggcttgggccgcggcgcctccaccttcaaggaggaaccgcagaccgtgccggaggcgcgcagccgggacgccacgccgccggtgtcccccatcaacatggaagaccaagagcgcatcaaagtggagcgcaagcggctgcggaaccggctggcggtcaccaagtgccggaagcggaagctggagcgcatcgcgcgcctggaggacaaggtgaagacgctcaaggccgagaacgcggggctgtcgagtaccgccggcctcctccgggagcaggtggcccagctcaaacagaaggtcatgacccacgtcagcaacggctgtcagctgctgcttggggtcaagggacacgccttc

SEQ ID NO: 96 - protein, 347 aa, Synthetic construct
MCTKMEQPFYHDDSYTATGYGRAPGGLSLHDYKLLKPSLAVNLADPYRSLKAPGARGPGPEGGGGGSYFSGQGSDTGASLKLASSELERLIVPNSNGVITTTPTPPGQYFYPRGGGSGGGAGGAGGGVTEEQEGFADGFVKALDDLHKMNHVTPPNVSLGATGGPPAGPGGVYAGPEPPPVYTNLSSYSPASASSGGAGAAVGTGSSYPTTTISYLPHAPPFAGGHPAQLGLGRGASTFKEEPQTVPEARSRDATPPVSPINMEDQERIKVERKRLRNRLAVTKCRKRKLERIARLEDKVKTLKAENAGLSSTAGLLREQVAQLKQKVMTHVSNGCQLLLGVKGHAF

SEQ ID NO: 97 - DNA, 1041 bp, Homo sapiens
  source 1..1041: mol_type = genomic DNA
atgtgcactaaaatggaacagcccttctaccacgacgactcatacacagctacgggatacggccgggcccctggtggcctctctctacacgactacaaactcctgaaaccgagcctggcggtcaacctggccgacccctaccggagtctcaaagcgcctggggctcgcggacccggcccagagggcggcggtggcggcagctacttttctggtcagggctcggacaccggcgcgtctctcaagctcgcctcttcggagctggaacgcctgattgtccccaacagcaacggcgtgatcacgacgacgcctacacccccgggacagtacttttacccccgcgggggtggcagcggtggaggtgcagggggcgcagggggcggcgtcaccgaggagcaggagggcttcgccgacggctttgtcaaagccctggacgatctgcacaagatgaaccacgtgacaccccccaacgtgtccctgggcgctaccggggggcccccggctgggcccgggggcgtctacgccggcccggagccacctcccgtttacaccaacctcagcagctactccccagcctctgcgtcctcgggaggcgccggggctgccgtcgggaccgggagctcgtacccgacgaccaccatcagctacctcccacacgcgccgcccttcgccggtggccacccggcgcagctgggcttgggccgcggcgcctccaccttcaaggaggaaccgcagaccgtgccggaggcgcgcagccgggacgccacgccgccggtgtcccccatcaacatggaagaccaagagcgcatcaaagtggagcgcaagcggctgcggaaccggctggcggccaccaagtgccggaagcggaagctggagcgcatcgcgcgcctggaggacaaggtgaagacgctcaaggccgagaacgcggggctgtcgagtaccgccggcctcctccgggagcaggtggcccagctcaaacagaaggtcatgacccacgtcagcaacggctgtcagctgctgcttggggtcaagggacacgccttc

SEQ ID NO: 98 - protein, 347 aa, Homo sapiens
MCTKMEQPFYHDDSYTATGYGRAPGGLSLHDYKLLKPSLAVNLADPYRSLKAPGARGPGPEGGGGGSYFSGQGSDTGASLKLASSELERLIVPNSNGVITTTPTPPGQYFYPRGGGSGGGAGGAGGGVTEEQEGFADGFVKALDDLHKMNHVTPPNVSLGATGGPPAGPGGVYAGPEPPPVYTNLSSYSPASASSGGAGAAVGTGSSYPTTTISYLPHAPPFAGGHPAQLGLGRGASTFKEEPQTVPEARSRDATPPVSPINMEDQERIKVERKRLRNRLAATKCRKRKLERIARLEDKVKTLKAENAGLSSTAGLLREQVAQLKQKVMTHVSNGCQLLLGVKGHAF

SEQ ID NO: 99 - DNA, 2028 bp, Synthetic construct
  source 1..2028: mol_type = other DNA
atggccgcggcctcctccccgcccagggccgagaggaagcgctggggttggggccgcctgccaggcgcccggcggggcagcgcgggcctggccaagaagtgccccttctcgctggagctggcggagggcggcccggcgggcggcgcgctctacgcgcccatcgcgcccggcgccccaggtcccgcgccccctgcgtccccggccgcgcccgccgcgcccccagttgcctccgaccttggcccgcggccgccggtgagcctagacccgcgcgtctccatctacagcacgcgccgcccggtgttggcgcgcacccacgtccagggccgcgtctacaacttcctcgagcgtcccaccggctggaaatgcttcgtttaccacttcgccgtcttcctcatcgtcctggtctgcctcatcttcagcgtgctgtccaccatcgagcagtatgccgccctggccacggggactctcttctggatggagatcgtgctggtggtgttcttcgggacggagtacgtggtccgcctctggtccgccggctgccgcagcaagtacgtgggcctctgggggcggctgcgctttgcccggaagcccatttccatcatcgacctcatcgtggtcgtggcctccatggtggtcctctgcgtgggctccaaggggcaggtgtttgccacgtcggccatcaggggcatccgcttcctgcagatcctgaggatgctacacgtcgaccgccagggaggcacctggaggctcctgggctccgtggtcttcatccaccgccaggagctgataaccaccctgtacatcggcttcctgggcctcatcttctcctcgtactttgtgtacctggctgagaaggacgcggtgaacgagtcaggccgcgtggagttcggcagctacgcagatgcgctgtggtggggggtggtcacagtcaccaccatcggctatggggacaaggtgccccagacgtgggtcgggaagaccatcgcctcctgcttctctgtctttgccatctccttctttgcgctcccagcggggattcttggctcggggtttgccctgaaggtgcagcagaagcagaggcagaagcacttcaaccggcagatcccggcggcagcctcactcattcagaccgcatggaggtgctatgctgccgagaaccccgactcctccacctggaagatctacatccggaaggccccccggagccacactctgctgtcacccagccccaaacccaagaagtctgtggtggtaaagaaaaaaaagttcaagctggacaaagacaatggggtgactcctggagagaagatgctcacagtcccccatatcacgtgcgaccccccagaagagcggcggctggaccacttctctgtcgacggctatgacagttctgtaaggaagagcccaacactgctggaagtgagcatgccccatttcatgagaaccaacagcttcgccgaggacctggacctggaaggggagactctgctgacacccatcacccacatctcacagctgcgggaacaccatcgggccaccattaaggtcattcgacgcatgcagtactttgtggccaagaagaaattccagcaagcgcggaagccttacgatgtgcgggacgtcattgagcagtactcgcagggccacctcaacctcatggtgcgcatcaaggagctgcagaggaggctggaccagtccattgggaagccctcactgttcatctccgtctcagaaaagagcaaggattgcggcagcaacacgatcggcgcccgcctgaaccgagtagaagacaaggtgacgcagctggaccagaggctggcactcatcaccgacatgcttcaccagctgctctccttgcacggtggcagcacccccggcagcggcggcccccccagagagggcggggcccacatcacccagccctgcggcagtggcggctccgtcgaccctgagctcttcctgcccagcaacaccctgcccacctacgagcagctgaccgtgcccaggaggggccccgatgaggggtcc

SEQ ID NO: 100 - protein, 676 aa, Synthetic construct
MAAASSPPRAERKRWGWGRLPGARRGSAGLAKKCPFSLELAEGGPAGGALYAPIAPGAPGPAPPASPAAPAAPPVASDLGPRPPVSLDPRVSIYSTRRPVLARTHVQGRVYNFLERPTGWKCFVYHFAVFLIVLVCLIFSVLSTIEQYAALATGTLFWMEIVLVVFFGTEYVVRLWSAGCRSKYVGLWGRLRFARKPISIIDLIVVVASMVVLCVGSKGQVFATSAIRGIRFLQILRMLHVDRQGGTWRLLGSVVFIHRQELITTLYIGFLGLIFSSYFVYLAEKDAVNESGRVEFGSYADALWWGVVTVTTIGYGDKVPQTWVGKTIASCFSVFAISFFALPAGILGSGFALKVQQKQRQKHFNRQIPAAASLIQTAWRCYAAENPDSSTWKIYIRKAPRSHTLLSPSPKPKKSVVVKKKKFKLDKDNGVTPGEKMLTVPHITCDPPEERRLDHFSVDGYDSSVRKSPTLLEVSMPHFMRTNSFAEDLDLEGETLLTPITHISQLREHHRATIKVIRRMQYFVAKKKFQQARKPYDVRDVIEQYSQGHLNLMVRIKELQRRLDQSIGKPSLFISVSEKSKDCGSNTIGARLNRVEDKVTQLDQRLALITDMLHQLLSLHGGSTPGSGGPPREGGAHITQPCGSGGSVDPELFLPSNTLPTYEQLTVPRRGPDEGS

SEQ ID NO: 101 - DNA, 2028 bp, Homo sapiens
  source 1..2028: mol_type = genomic DNA
atggccgcggcctcctccccgcccagggccgagaggaagcgctggggttggggccgcctgccaggcgcccggcggggcagcgcgggcctggccaagaagtgccccttctcgctggagctggcggagggcggcccggcgggcggcgcgctctacgcgcccatcgcgcccggcgccccaggtcccgcgccccctgcgtccccggccgcgcccgccgcgcccccagttgcctccgaccttggcccgcggccgccggtgagcctagacccgcgcgtctccatctacagcacgcgccgcccggtgttggcgcgcacccacgtccagggccgcgtctacaacttcctcgagcgtcccaccggctggaaatgcttcgtttaccacttcgccgtcttcctcatcgtcctggtctgcctcatcttcagcgtgctgtccaccatcgagcagtatgccgccctggccacggggactctcttctggatggagatcgtgctggtggtgttcttcgggacggagtacgtggtccgcctctggtccgccggctgccgcagcaagtacgtgggcctctgggggcggctgcgctttgcccggaagcccatttccatcatcgacctcatcgtggtcgtggcctccatggtggtcctctgcgtgggctccaaggggcaggtgtttgccacgtcggccatcaggggcatccgcttcctgcagatcctgaggatgctacacgtcgaccgccagggaggcacctggaggctcctgggctccgtggtcttcatccaccgccaggagctgataaccaccctgtacatcggcttcctgggcctcatcttctcctcgtactttgtgtacctggctgagaaggacgcggtgaacgagtcaggccgcgtggagttcggcagctacgcagatgcgctgtggtggggggtggtcacagtcaccaccatcggctatggggacaaggtgccccagacgtgggtcgggaagaccatcgcctcctgcttctctgtctttgccatctccttctttgcgctcccagcggggattcttggctcggggtttgccctgaaggtgcagcagaagcagaggcagaagcacttcaaccggcagatcccggcggcagcctcactcattcagaccgcatggaggtgctatgctgccgagaaccccgactcctccacctggaagatctacatccggaaggccccccggagccacactctgctgtcacccagccccaaacccaagaagtctgtggtggtaaagaaaaaaaagttcaagctggacaaagacaatggggtgactcctggagagaagatgctcacagtcccccatatcacgtgcgaccccccagaagagcggcggctggaccacttctctgtcgacggctatgacagttctgtaaggaagagcccaacactgctggaagtgagcatgccccatttcatgagaaccaacagcttcgccgaggacctggacctggaaggggagactctgctgacacccatcacccacatctcacagctgcgggaacaccatcgggccaccattaaggtcattcgacgcatgcagtactttgtggccaagaagaaattccagcaagcgcggaagccttacgatgtgcgggacgtcattgagcagtactcgcagggccacctcaacctcatggtgcgcatcaaggagctgcagaggaggctggaccagtccattgggaagccctcactgttcatctccgtctcagaaaagagcaaggatcgcggcagcaacacgatcggcgcccgcctgaaccgagtagaagacaaggtgacgcagctggaccagaggctggcactcatcaccgacatgcttcaccagctgctctccttgcacggtggcagcacccccggcagcggcggcccccccagagagggcggggcccacatcacccagccctgcggcagtggcggctccgtcgaccctgagctcttcctgcccagcaacaccctgcccacctacgagcagctgaccgtgcccaggaggggccccgatgaggggtcc

SEQ ID NO: 102 - protein, 676 aa, Homo sapiens [sequence omitted]

SEQ ID NO: 103 - DNA, 3390 bp, Synthetic construct
  source 1..3390: mol_type = other DNA
atgaagaggagtgaaaagccagaaggatatagacaaatgaggcctaagacctttcctgccagtaactatactgtcagtagccggcaaatgttacaagaaattcgggaatcccttaggaatttatctaaaccatctgatgctgctaaggctgagcataacatgagtaaaatgtcaaccgaagatcctcgacaagtcagaaatccacccaaatttgggacgcatcataaagccttgcaggaaattcgaaactctctgcttccatttgcaaatgaaacaaattcttctcggagtacttcagaagttaatccacaaatgcttcaagacttgcaagctgctggatttgatgaggatatggttatacaagctcttcagaaaactaacaacagaagtatagaagcagcaattgaattcattagtaaaatgagttaccaagatcctcgacgagagcagatggctgcagcagctgccagacctattaatgccagcatgaaaacagggaatgtgcagcaatcagttaaccgcaaacagagctggaaaggttctaaagaatccttagttcctcagaggcatggcccgccactaggagaaagtgtggcctatcattctgagagtcccaactcacagacagatgtaggaagacctttgtctggatctggtatatcagcatttgttcaagctcaccctagcaacggacagagagtgaaccccccaccaccacctcaagtaaggagtgttactcctccaccacctccaagaggccagactccccctccaagaggtacaactccacctcccccttcatgggaaccaaactctcaaacaaagcgctattctggaaacatggaatacgtaatctcccgaatctctcctgtcccacctggggcatggcaagagggctatcctccaccacctctcaacacttcccccatgaatcctcctaatcaaggacagagaggcattagttctgttcctgttggcagacaaccaatcatcatgcagagttctagcaaatttaactttccatcagggagacctggaatgcagaatggtactggacaaactgatttcatgatacaccaaaatgttgtccctgctggcactgtgaatcggcagccaccacctccatatcctctgacagcagctaatggacaaagcccttctgctttacaaacagggggatctgctgctccttcgtcatatacaaatggaagtattcctcagtctatgatggtgccaaacagaaatagtcataacatggaactatataacattagtgtacctggactgcaaacaaattggcctcagtcatcttctgctccagcccagtcatccccgagcagtgggcatgaaatccctacatggcaacctaacataccagtgaggtcaaattcttttaataacccattaggaaatagagcaagtcactctgctaattctcagccttctgctacaacagtcactgcaattacaccagctcctattcaacagcctgtgaaaagtatgcgtgtattaaaaccagagctacagactgctttagcacctacacacccttcttggataccacagccaattcaaactgttcaacccagtccttttcctgagggaaccgcttcaaatgtgactgtgatgccacctgttgctgaagctccaaactatcaaggaccaccaccaccctacccaaaacatctgctgcaccaaaacccatctgttcctccatacgagtcaatcagtaagcctagcaaagaggatcagccaagcttgcccaaggaagatgagagtgaaaagagttatgaaaatgttgatagtggggataaagaaaagaaacagattacaacttcacctattactgttaggaaaaacaagaaagatgaagagcgaagggaatctcgtattcaaagttattctcctcaagcatttaaattctttatggagcaacatgtagaaaatgtactcaaatctcatcagcagcgtctacatcgtaaaaaacaattagagaatgaaatgatgcgggttggattatctcaagatgcccaggatcaaatgagaaagatgctttgccaaaaagaatctaattacatccgtcttaaaagggctaaaatggacaagtctatgtttgtgaagataaagacactaggaataggagcatttggtgaagtctgtctagcaagaaaagtagatactaaggctttgtatgcaacaaaaactcttcgaaagaaagatgttcttcttcgaaatcaagtcgctcatgttaaggctgagagagatatcctggctgaagctgacaatgaatgggtagttcgtctatattattcattccaagataaggacaatttatactttgtaatggactacattcctgggggtgatatgatgagcctattaattagaatgggcatctttccagaaagtctggcacgattctacatagcagaacttacctgtgcagttgaaagtgttcataaaatgggttttattcatagagatattaaacctgataatattttgattgatcgtgatggtcatattaaattgactgactttggcctctgcactggcttcagatggacacacgattctaagtactatcagagtggtgaccatccacggcaagatagcatggatttcagtaatgaatggggggatccctcaagctgtcgatgtggagacagactgaagccattagagcggagagctgcacgccagcaccagcgatgtctagcacattctttggttgggactcccaattatattgcacctgaagtgttgctacgaacaggatacacacagttgtgtgattggtggagtgttggtgttattctttttgaaatgttggtgggacaacctcctttcttggcacaaacaccattagaaacacaaatgaaggttatcaactggcaaacatctcttcacattccaccacaagctaaactcagtcctgaagcttctgatcttattattaaactttgccgaggacccgaagatcgcttaggcaagaatggtgctgatgaaataaaagctcatccattttttaaaacaattgacttctccagtgacctgagacagcagtctgcttcatacattcctaaaatcacacacccaacagatacatcaaattttgatcctgttgatcctgataaattatggagtgatgataacgaggaagaaaatgtaaatgacactctcaatggatggtataaaaatggaaagcatcctgaacatgcattctatgaatttaccttccgaaggttttttgatgacaatggctacccatataattatccgaagcctattgaatatgaatacattaattcacaaggctcagagcagcagtcggatgaagatgatcaaaacacaggctcagagattaaaaatcgcgatctagtatatgtt

SEQ ID NO: 104 - protein, 1130 aa, Synthetic construct
MKRSEKPEGYRQMRPKTFPASNYTVSSRQMLQEIRESLRNLSKPSDAAKAEHNMSKMSTEDPRQVRNPPKFGTHHKALQEIRNSLLPFANETNSSRSTSEVNPQMLQDLQAAGFDEDMVIQALQKTNNRSIEAAIEFISKMSYQDPRREQMAAAAARPINASMKTGNVQQSVNRKQSWKGSKESLVPQRHGPPLGESVAYHSESPNSQTDVGRPLSGSGISAFVQAHPSNGQRVNPPPPPQVRSVTPPPPPRGQTPPPRGTTPPPPSWEPNSQTKRYSGNMEYVISRISPVPPGAWQEGYPPPPLNTSPMNPPNQGQRGISSVPVGRQPIIMQSSSKFNFPSGRPGMQNGTGQTDFMIHQNVVPAGTVNRQPPPPYPLTAANGQSPSALQTGGSAAPSSYTNGSIPQSMMVPNRNSHNMELYNISVPGLQTNWPQSSSAPAQSSPSSGHEIPTWQPNIPVRSNSFNNPLGNRASHSANSQPSATTVTAITPAPIQQPVKSMRVLKPELQTALAPTHPSWIPQPIQTVQPSPFPEGTASNVTVMPPVAEAPNYQGPPPPYPKHLLHQNPSVPPYESISKPSKEDQPSLPKEDESEKSYENVDSGDKEKKQITTSPITVRKNKKDEERRESRIQSYSPQAFKFFMEQHVENVLKSHQQRLHRKKQLENEMMRVGLSQDAQDQMRKMLCQKESNYIRLKRAKMDKSMFVKIKTLGIGAFGEVCLARKVDTKALYATKTLRKKDVLLRNQVAHVKAERDILAEADNEWVVRLYYSFQDKDNLYFVMDYIPGGDMMSLLIRMGIFPESLARFYIAELTCAVESVHKMGFIHRDIKPDNILIDRDGHIKLTDFGLCTGFRWTHDSKYYQSGDHPRQDSMDFSNEWGDPSSCRCGDRLKPLERRAARQHQRCLAHSLVGTPNYIAPEVLLRTGYTQLCDWWSVGVILFEMLVGQPPFLAQTPLETQMKVINWQTSLHIPPQAKLSPEASDLIIKLCRGPEDRLGKNGADEIKAHPFFKTIDFSSDLRQQSASYIPKITHPTDTSNFDPVDPDKLWSDDNEEENVNDTLNGWYKNGKHPEHAFYEFTFRRFFDDNGYPYNYPKPIEYEYINSQGSEQQSDEDDQNTGSEIKNRDLVYV

SEQ ID NO: 105 - DNA, 3390 bp, Homo sapiens
  source 1..3390: mol_type = genomic DNA
atgaagaggagtgaaaagccagaaggatatagacaaatgaggcctaagacctttcctgccagtaactatactgtcagtagccggcaaatgttacaagaaattcgggaatcccttaggaatttatctaaaccatctgatgctgctaaggctgagcataacatgagtaaaatgtcaaccgaagatcctcgacaagtcagaaatccacccaaatttgggacgcatcataaagccttgcaggaaattcgaaactctctgcttccatttgcaaatgaaacaaattcttctcggagtacttcagaagttaatccacaaatgcttcaagacttgcaagctgctggatttgatgaggatatggttatacaagctcttcagaaaactaacaacagaagtatagaagcagcaattgaattcattagtaaaatgagttaccaagatcctcgacgagagcagatggctgcagcagctgccagacctattaatgccagcatgaaaccagggaatgtgcagcaatcagttaaccgcaaacagagctggaaaggttctaaagaatccttagttcctcagaggcatggcccgccactaggagaaagtgtggcctatcattctgagagtcccaactcacagacagatgtaggaagacctttgtctggatctggtatatcagcatttgttcaagctcaccctagcaacggacagagagtgaaccccccaccaccacctcaagtaaggagtgttactcctccaccacctccaagaggccagactccccctccaagaggtacaactccacctcccccttcatgggaaccaaactctcaaacaaagcgctattctggaaacatggaatacgtaatctcccgaatctctcctgtcccacctggggcatggcaagagggctatcctccaccacctctcaacacttcccccatgaatcctcctaatcaaggacagagaggcattagttctgttcctgttggcagacaaccaatcatcatgcagagttctagcaaatttaactttccatcagggagacctggaatgcagaatggtactggacaaactgatttcatgatacaccaaaatgttgtccctgctggcactgtgaatcggcagccaccacctccatatcctctgacagcagctaatggacaaagcccttctgctttacaaacagggggatctgctgctccttcgtcatatacaaatggaagtattcctcagtctatgatggtgccaaacagaaatagtcataacatggaactatataacattagtgtacctggactgcaaacaaattggcctcagtcatcttctgctccagcccagtcatccccgagcagtgggcatgaaatccctacatggcaacctaacataccagtgaggtcaaattcttttaataacccattaggaaatagagcaagtcactctgctaattctcagccttctgctacaacagtcactgcaattacaccagctcctattcaacagcctgtgaaaagtatgcgtgtattaaaaccagagctacagactgctttagcacctacacacccttcttggataccacagccaattcaaactgttcaacccagtccttttcctgagggaaccgcttcaaatgtgactgtgatgccacctgttgctgaagctccaaactatcaaggaccaccaccaccctacccaaaacatctgctgcaccaaaacccatctgttcctccatacgagtcaatcagtaagcctagcaaagaggatcagccaagcttgcccaaggaagatgagagtgaaaagagttatgaaaatgttgatagtggggataaagaaaagaaacagattacaacttcacctattactgttaggaaaaacaagaaagatgaagagcgaagggaatctcgtattcaaagttattctcctcaagcatttaaattctttatggagcaacatgtagaaaatgtactcaaatctcatcagcagcgtctacatcgtaaaaaacaattagagaatgaaatgatgcgggttggattatctcaagatgcccaggatcaaatgagaaagatgctttgccaaaaagaatctaattacatccgtcttaaaagggctaaaatggacaagtctatgtttgtgaagataaagacactaggaataggagcatttggtgaagtctgtctagcaagaaaagtagatactaaggctttgtatgcaacaaaaactcttcgaaagaaagatgttcttcttcgaaatcaagtcgctcatgttaaggctgagagagatatcctggctgaagctgacaatgaatgggtagttcgtctatattattcattccaagataaggacaatttatactttgtaatggactacattcctgggggtgatatgatgagcctattaattagaatgggcatctttccagaaagtctggcacgattctacatagcagaacttacctgtgcagttgaaagtgttcataaaatgggttttattcatagagatattaaacctgataatattttgattgatcgtgatggtcatattaaattgactgactttggcctctgcactggcttcagatggacacacgattctaagtactatcagagtggtgaccatccacggcaagatagcatggatttcagtaatgaatggggggatccctcaagctgtcgatgtggagacagactgaagccattagagcggagagctgcacgccagcaccagcgatgtctagcacattctttggttgggactcccaattatattgcacctgaagtgttgctacgaacaggatacacacagttgtgtgattggtggagtgttggtgttattctttttgaaatgttggtgggacaacctcctttcttggcacaaacaccattagaaacacaaatgaaggttatcaactggcaaacatctcttcacattccaccacaagctaaactcagtcctgaagcttctgatcttattattaaactttgccgaggacccgaagatcgcttaggcaagaatggtgctgatgaaataaaagctcatccattttttaaaacaattgacttctccagtgacctgagacagcagtctgcttcatacattcctaaaatcacacacccaacagatacatcaaattttgatcctgttgatcctgataaattatggagtgatgataacgaggaagaaaatgtaaatgacactctcaatggatggtataaaaatggaaagcatcctgaacatgcattctatgaatttaccttccgaaggttttttgatgacaatggctacccatataattatccgaagcctattgaatatgaatacattaattcacaaggctcagagcagcagtcggatgaagatgatcaaaacacaggctcagagattaaaaatcgcgatctagtatatgtt

SEQ ID NO: 106 - protein, 1130 aa, Homo sapiens
MKRSEKPEGYRQMRPKTFPASNYTVSSRQMLQEIRESLRNLSKPSDAAKAEHNMSKMSTEDPRQVRNPPKFGTHHKALQEIRNSLLPFANETNSSRSTSEVNPQMLQDLQAAGFDEDMVIQALQKTNNRSIEAAIEFISKMSYQDPRREQMAAAAARPINASMKPGNVQQSVNRKQSWKGSKESLVPQRHGPPLGESVAYHSESPNSQTDVGRPLSGSGISAFVQAHPSNGQRVNPPPPPQVRSVTPPPPPRGQTPPPRGTTPPPPSWEPNSQTKRYSGNMEYVISRISPVPPGAWQEGYPPPPLNTSPMNPPNQGQRGISSVPVGRQPIIMQSSSKFNFPSGRPGMQNGTGQTDFMIHQNVVPAGTVNRQPPPPYPLTAANGQSPSALQTGGSAAPSSYTNGSIPQSMMVPNRNSHNMELYNISVPGLQTNWPQSSSAPAQSSPSSGHEIPTWQPNIPVRSNSFNNPLGNRASHSANSQPSATTVTAITPAPIQQPVKSMRVLKPELQTALAPTHPSWIPQPIQTVQPSPFPEGTASNVTVMPPVAEAPNYQGPPPPYPKHLLHQNPSVPPYESISKPSKEDQPSLPKEDESEKSYENVDSGDKEKKQITTSPITVRKNKKDEERRESRIQSYSPQAFKFFMEQHVENVLKSHQQRLHRKKQLENEMMRVGLSQDAQDQMRKMLCQKESNYIRLKRAKMDKSMFVKIKTLGIGAFGEVCLARKVDTKALYATKTLRKKDVLLRNQVAHVKAERDILAEADNEWVVRLYYSFQDKDNLYFVMDYIPGGDMMSLLIRMGIFPESLARFYIAELTCAVESVHKMGFIHRDIKPDNILIDRDGHIKLTDFGLCTGFRWTHDSKYYQSGDHPRQDSMDFSNEWGDPSSCRCGDRLKPLERRAARQHQRCLAHSLVGTPNYIAPEVLLRTGYTQLCDWWSVGVILFEMLVGQPPFLAQTPLETQMKVINWQTSLHIPPQAKLSPEASDLIIKLCRGPEDRLGKNGADEIKAHPFFKTIDFSSDLRQQSASYIPKITHPTDTSNFDPVDPDKLWSDDNEEENVNDTLNGWYKNGKHPEHAFYEFTFRRFFDDNGYPYNYPKPIEYEYINSQGSEQQSDEDDQNTGSEIKNRDLVYV

SEQ ID NO: 107 - DNA, 618 bp, Synthetic construct
  source 1..618: mol_type = other DNA
atgagcacgggctccgtaagcgaccctgaggagatggagcttcgcggtctccaacgagaataccctgttcccgcgagtaagagaccgccgctgcggggtgttgaaagatcctatgcatctccctctgataactcctctgcggaggaggaggatcctgatggggaagaggagcgctgcgcattgggcaccgctggctcagctgaaggttgcaaaaggaagaggccacgcgttgcaggtggaggcggtgcgggtggctcagcaggcggtggtgggaaaaaaccccttcccgctaagggttctgccgccgagtgtaagcagtcccaacgaaatgctgcgaatgcacggaaaagggccagaatgagagtgcttagcaaagcctttagccggttgaagacatcactcccttgggtcccgccagatacaaagctcagtaagcttgataccttgagacttgcctcctcatatatcgctcacctgagacaactgcttcaggaggacaggtacgagaatggatacgttcacccggtaaatcttacttggcctttcgtcgtctcaggaagaccggattccgataccaaagaggtatccgccgcgaaccggttgtgcgggaccacggca

SEQ ID NO: 108 - protein, 206 aa, Synthetic construct
MSTGSVSDPEEMELRGLQREYPVPASKRPPLRGVERSYASPSDNSSAEEEDPDGEEERCALGTAGSAEGCKRKRPRVAGGGGAGGSAGGGGKKPLPAKGSAAECKQSQRNAANARKRARMRVLSKAFSRLKTSLPWVPPDTKLSKLDTLRLASSYIAHLRQLLQEDRYENGYVHPVNLTWPFVVSGRPDSDTKEVSAANRLCGTTA

SEQ ID NO: 109 - DNA, 618 bp, Homo sapiens
  source 1..618: mol_type = genomic DNA
atgagcacgggctccgtaagcgaccctgaggagatggagcttcgcggtctccaacgagaataccctgttcccgcgagtaagagaccgccgctgcggggtgttgaaagatcctatgcatctccctctgataactcctctgcggaggaggaggatcctgatggggaagaggagcgctgcgcattgggcaccgctggctcagctgaaggttgcaaaaggaagaggccacgcgttgcaggtggaggcggtgcgggtggctcagcaggcggtggtgggaaaaaaccccttcccgctaagggttctgccgccgagtgtaagcagtcccaacgaaatgctgcgaatgcacgggaaagggccagaatgagagtgcttagcaaagcctttagccggttgaagacatcactcccttgggtcccgccagatacaaagctcagtaagcttgataccttgagacttgcctcctcatatatcgctcacctgagacaactgcttcaggaggacaggtacgagaatggatacgttcacccggtaaatcttacttggcctttcgtcgtctcaggaagaccggattccgataccaaagaggtatccgccgcgaaccggttgtgcgggaccacggca

SEQ ID NO: 110 - protein, 206 aa, Homo sapiens
MSTGSVSDPEEMELRGLQREYPVPASKRPPLRGVERSYASPSDNSSAEEEDPDGEEERCALGTAGSAEGCKRKRPRVAGGGGAGGSAGGGGKKPLPAKGSAAECKQSQRNAANARERARMRVLSKAFSRLKTSLPWVPPDTKLSKLDTLRLASSYIAHLRQLLQEDRYENGYVHPVNLTWPFVVSGRPDSDTKEVSAANRLCGTTA

SEQ ID NO: 111 - DNA, 1392 bp, Synthetic construct
  source 1..1392: mol_type = other DNA
atgccgagctgctccacgtccaccatgccgggcatgatctgcaagaacccagacctcgagtttgactcgctacagccctgcttctacccggacgaagatgacttctacttcggcggccccgactcgaccctcccgggggaggacatctggaagaagtttgagctgctgcccacgcccccgctgtcgcccagccgtggcttcgcggagcacagctccgagcccccgagctgggtcacggagatgctgcttgagaacgagctgtggggcagcccggccgaggaggacgcgttcggcctggggggactgggtggcctcacccccaacccggtcatcctccaggactgcatgtggagcggcttctccgcccgcgagaagctggagcgcgccgtgagcgagaagctgcagcacggccgcgggccgccaaccgccggttccaccgcccagtccccgggagccggcgccgccagccctgcgggtcgcgggcacggcggggctgcgggagccggccgcgccggggccgccctgcccgccgagctcgcccacccggccgccgagtgcgtggatcccgccgtggtcttcccctttcccgtgaacaagcgcgagccagcgcccgtgcccgcagccccggccagtgccccggcggcgggccctgcggtcgcctcgggggcgggtattgccgccccagccggggccccgggggtcgcccctccgcgcccaggcggccgccagaccagcggcggcgaccacaaggccctcagtacctccggagaggacaccctgagcgattcagatgatgaagatgatgaagaggaagatgaagaggaagaaatcgacgtggtcactgtggagaagcggcgttcctcctccaacaccaaggctgtcaccacattcaccatcactgtgcgtcccaagaacgcagccctgggtcccgggagggctcagtccagcgagctgatcctcaaacgatgccttcccatccaccagcagcacaactatgccgccccctctccctacgtggagagtgaggatgcacccccacagaagaagataaagagcgaggcgtccccacgtccgctcaagagtgtcatccccccaaaggctaagagcttgagcccccgaaactctgactcggaggacagtgagcgtcgcagaaaccacaacatcctggagcgccagcgccgcaacgaccttcggtccagctttctcacgctcagggaccacgtgccggagttggtaaagaatgagaaggccgccaaggtggtcattttgaaaaaggccactgagtatgtccactccctccaggccgaggagcaccagcttttgctggaaaaggaaaaattgcaggcaagacagcagcagttgctaaagaaaattgaacacgctcggacttgc

SEQ ID NO: 112 - protein, 464 aa, Synthetic construct
MPSCSTSTMPGMICKNPDLEFDSLQPCFYPDEDDFYFGGPDSTLPGEDIWKKFELLPTPPLSPSRGFAEHSSEPPSWVTEMLLENELWGSPAEEDAFGLGGLGGLTPNPVILQDCMWSGFSAREKLERAVSEKLQHGRGPPTAGSTAQSPGAGAASPAGRGHGGAAGAGRAGAALPAELAHPAAECVDPAVVFPFPVNKREPAPVPAAPASAPAAGPAVASGAGIAAPAGAPGVAPPRPGGRQTSGGDHKALSTSGEDTLSDSDDEDDEEEDEEEEIDVVTVEKRRSSSNTKAVTTFTITVRPKNAALGPGRAQSSELILKRCLPIHQQHNYAAPSPYVESEDAPPQKKIKSEASPRPLKSVIPPKAKSLSPRNSDSEDSERRRNHNILERQRRNDLRSSFLTLRDHVPELVKNEKAAKVVILKKATEYVHSLQAEEHQLLLEKEKLQARQQQLLKKIEHARTC

SEQ ID NO: 113 - DNA, 1392 bp, Homo sapiens
  source 1..1392: mol_type = genomic DNA
atgccgagctgctccacgtccaccatgccgggcatgatctgcaagaacccagacctcgagtttgactcgctacagccctgcttctacccggacgaagatgacttctacttcggcggccccgactcgacccccccgggggaggacatctggaagaagtttgagctgctgcccacgcccccgctgtcgcccagccgtggcttcgcggagcacagctccgagcccccgagctgggtcacggagatgctgcttgagaacgagctgtggggcagcccggccgaggaggacgcgttcggcctggggggactgggtggcctcacccccaacccggtcatcctccaggactgcatgtggagcggcttctccgcccgcgagaagctggagcgcgccgtgagcgagaagctgcagcacggccgcgggccgccaaccgccggttccaccgcccagtccccgggagccggcgccgccagccctgcgggtcgcgggcacggcggggctgcgggagccggccgcgccggggccgccctgcccgccgagctcgcccacccggccgccgagtgcgtggatcccgccgtggtcttcccctttcccgtgaacaagcgcgagccagcgcccgtgcccgcagccccggccagtgccccggcggcgggccctgcggtcgcctcgggggcgggtattgccgccccagccggggccccgggggtcgcccctccgcgcccaggcggccgccagaccagcggcggcgaccacaaggccctcagtacctccggagaggacaccctgagcgattcagatgatgaagatgatgaagaggaagatgaagaggaagaaatcgacgtggtcactgtggagaagcggcgttcctcctccaacaccaaggctgtcaccacattcaccatcactgtgcgtcccaagaacgcagccctgggtcccgggagggctcagtccagcgagctgatcctcaaacgatgccttcccatccaccagcagcacaactatgccgccccctctccctacgtggagagtgaggatgcacccccacagaagaagataaagagcgaggcgtccccacgtccgctcaagagtgtcatccccccaaaggctaagagcttgagcccccgaaactctgactcggaggacagtgagcgtcgcagaaaccacaacatcctggagcgccagcgccgcaacgaccttcggtccagctttctcacgctcagggaccacgtgccggagttggtaaagaatgagaaggccgccaaggtggtcattttgaaaaaggccactgagtatgtccactccctccaggccgaggagcaccagcttttgctggaaaaggaaaaattgcaggcaagacagcagcagttgctaaagaaaattgaacacgctcggacttgc

SEQ ID NO: 114 - protein, 464 aa, Homo sapiens
MPSCSTSTMPGMICKNPDLEFDSLQPCFYPDEDDFYFGGPDSTPPGEDIWKKFELLPTPPLSPSRGFAEHSSEPPSWVTEMLLENELWGSPAEEDAFGLGGLGGLTPNPVILQDCMWSGFSAREKLERAVSEKLQHGRGPPTAGSTAQSPGAGAASPAGRGHGGAAGAGRAGAALPAELAHPAAECVDPAVVFPFPVNKREPAPVPAAPASAPAAGPAVASGAGIAAPAGAPGVAPPRPGGRQTSGGDHKALSTSGEDTLSDSDDEDDEEEDEEEEIDVVTVEKRRSSSNTKAVTTFTITVRPKNAALGPGRAQSSELILKRCLPIHQQHNYAAPSPYVESEDAPPQKKIKSEASPRPLKSVIPPKAKSLSPRNSDSEDSERRRNHNILERQRRNDLRSSFLTLRDHVPELVKNEKAAKVVILKKATEYVHSLQAEEHQLLLEKEKLQARQQQLLKKIEHARTC

SEQ ID NO: 115 - DNA, 2910 bp, Synthetic construct
  source 1..2910: mol_type = other DNA
atggcagaagatgatccatatttgggaaggcctgaacaaatgtttcatttggatccttctttgactcatacaatatttaatccagaagtatttcaaccacagatggcactgccaacagcagatggcccataccttcaaatattagagcaacctaaacagagaggatttcgtttccgttatgtatgtgaaggcccatcctatggtggactacctggtgcctctagtgaaaagaacaagaagtcttaccctcaggtcaaaatctgcaactatgtgggaccagcaaaggttattgttcagttggtcacaaatggaaaaaatatccacctgcatgcccacagcctggtgggaaaacactgtgaggatgggatctgcactgtaactgctggacccaaggacatggtggtcggcttcgcaaacctgggtatacttcatgtgacaaagaaaaaagtatttgaaacactggaagcacgaatgacagaggcgtgtataaggggccataatcctggactcttggtgcaccctgaccttgcctatttgcaagcagaaggtggaggggaccggcagctgggagatcgggaaaaagagctaatccgccaagcagctctgcagcagaccaaggagatggacctcagcgtggtgcggctcatgtttacagcttttcttccggatagcactggcagcttcacaaggcgcctggaacccgtggtatcagacgccatctatgacagtaaagcccccaatgcatccaacttgaaaattgtaagaatggacaggacagctggatgtgtgactggaggggaggaaatttatcttctttgtgacaaagttcagaaagatgacatccagattcgattttatgaagaggaagaaaatggtggagtctgggaaggatttggagatttttcccccacagatgttcatagacaatttgccattgtcttcaaaactccaaagtataaagatattaatattacaaaaccagcctctgtgtttgtccagcttcggaggaaatctgacttggaaactagtgaaccaaaacctttcctctactatcctgaaatcaaagataaagaagaagtgcagaggaaacgtcagaagctcatgcccaatttttcggatagtttcggcggtggtagtggtgccggagctggaggcggaggcatgtttggtagtggcggtggaggagggggcactggaagtacaggtccagggtatagcttcccacactatggatttcctacttatggtgggattactttccatcctggaactactaaatctaatgctgggatgaagcatggaaccatggacactgaatctaaaaaggaccctgaaggttgtgacaaaagtgatgacaaaaacactgtaaacctctttgggaaagttattgaaaccacagagcaagatcaggagcccagcgaggccaccgttgggaatggtgaggtcactctaacgtatgcaacaggaacaaaagaagagagtgctggagttcaggataacctctttctagagaaggctatgcagcttgcaaagaggcatgccaatgcccttttcgactacgcggtgacaggagacgtgaagatgctgctggccgtccagcgccatctcactgctgtgcaggatgagaatggggacagtgtcttacacttagcaatcatccaccttcattctcaacttgtgagggatctactagaagtcacatctggtttgatttctgatgacattatcaacatgagaaatgatctgtaccagacgcccttgcacttggcagtgatcactaagcaggaagatgtggtggaggatttgctgagggctggggccgacctgagccttctggaccgcttgggtaactctgttttgcacctagctgccaaagaaggacatgataaagttctcagtatcttactcaagcacaaaaaggcagcactacttcttgaccaccccaacggggacggtctgaatgccattcatctagccatgatgagcaatagcctgccatgtttgctgctgctggtggccgctggggctgacgtcaatgctcaggagcagaagtccgggcgcacagcactgcacctggctgtggagcacgacaacatctcattggcaggctgcctgctcctggagggtgatgcccatgtggacagtactacctacgatggaaccacacccctgcatatagcagctgggagagggtccaccaggctggcagctcttctcaaagcagcaggagcagatcccctggtggagaactttgagcctctctatgacctggatgactcttgggaaaatgcaggagaggatgaaggagttgtgcctggaaccacgcctctagatatggccaccagctggcaggtatttgacatattaaatgggaaaccatatgagccagagtttacatctgatgatttactagcacaaggagacatgaaacagctggctgaagatgtgaagctgcagctgtataagttactagaaattcctgatccagacaaaaactgggctactctggcgcagaaattaggtctggggatacttaataatgccttccggctgagtcctgctccttccaaaacacttatggacaactatgaggtctctgggggtacagtcagagagctggtggaggccctgagacaaatgggctacaccgaagcaattgaagtgatccaggcagcctccagcccagtgaagaccacctctcaggcccactcgctgcctctctcgcctgcctccacaaggcagcaaatagacgagctccgagacagtgacagtgtctgcgacagcggcgtggagacatccttccgcaaactcagctttaccgagtctctgaccagtggtgcctcactgctaactctcaacaaaatgccccatgattatgggcaggaaggacctctagaaggcaaaattttg

SEQ ID NO: 116 - protein, 970 aa, Synthetic construct
MAEDDPYLGRPEQMFHLDPSLTHTIFNPEVFQPQMALPTADGPYLQILEQPKQRGFRFRYVCEGPSYGGLPGASSEKNKKSYPQVKICNYVGPAKVIVQLVTNGKNIHLHAHSLVGKHCEDGICTVTAGPKDMVVGFANLGILHVTKKKVFETLEARMTEACIRGHNPGLLVHPDLAYLQAEGGGDRQLGDREKELIRQAALQQTKEMDLSVVRLMFTAFLPDSTGSFTRRLEPVVSDAIYDSKAPNASNLKIVRMDRTAGCVTGGEEIYLLCDKVQKDDIQIRFYEEEENGGVWEGFGDFSPTDVHRQFAIVFKTPKYKDINITKPASVFVQLRRKSDLETSEPKPFLYYPEIKDKEEVQRKRQKLMPNFSDSFGGGSGAGAGGGGMFGSGGGGGGTGSTGPGYSFPHYGFPTYGGITFHPGTTKSNAGMKHGTMDTESKKDPEGCDKSDDKNTVNLFGKVIETTEQDQEPSEATVGNGEVTLTYATGTKEESAGVQDNLFLEKAMQLAKRHANALFDYAVTGDVKMLLAVQRHLTAVQDENGDSVLHLAIIHLHSQLVRDLLEVTSGLISDDIINMRNDLYQTPLHLAVITKQEDVVEDLLRAGADLSLLDRLGNSVLHLAAKEGHDKVLSILLKHKKAALLLDHPNGDGLNAIHLAMMSNSLPCLLLLVAAGADVNAQEQKSGRTALHLAVEHDNISLAGCLLLEGDAHVDSTTYDGTTPLHIAAGRGSTRLAALLKAAGADPLVENFEPLYDLDDSWENAGEDEGVVPGTTPLDMATSWQVFDILNGKPYEPEFTSDDLLAQGDMKQLAEDVKLQLYKLLEIPDPDKNWATLAQKLGLGILNNAFRLSPAPSKTLMDNYEVSGGTVRELVEALRQMGYTEAIEVIQAASSPVKTTSQAHSLPLSPASTRQQIDELRDSDSVCDSGVETSFRKLSFTESLTSGASLLTLNKMPHDYGQEGPLEGKIL

SEQ ID NO: 117 - DNA, 2910 bp, Homo sapiens
  source 1..2910: mol_type = genomic DNA
atggcagaagatgatccatatttgggaaggcctgaacaaatgtttcatttggatccttctttgactcatacaatatttaatccagaagtatttcaaccacagatggcactgccaacagcagatggcccataccttcaaatattagagcaacctaaacagagaggatttcgtttccgttatgtatgtgaaggcccatcccatggtggactacctggtgcctctagtgaaaagaacaagaagtcttaccctcaggtcaaaatctgcaactatgtgggaccagcaaaggttattgttcagttggtcacaaatggaaaaaatatccacctgcatgcccacagcctggtgggaaaacactgtgaggatgggatctgcactgtaactgctggacccaaggacatggtggtcggcttcgcaaacctgggtatacttcatgtgacaaagaaaaaagtatttgaaacactggaagcacgaatgacagaggcgtgtataaggggccataatcctggactcttggtgcaccctgaccttgcctatttgcaagcagaaggtggaggggaccggcagctgggagatcgggaaaaagagctaatccgccaagcagctctgcagcagaccaaggagatggacctcagcgtggtgcggctcatgtttacagcttttcttccggatagcactggcagcttcacaaggcgcctggaacccgtggtatcagacgccatctatgacagtaaagcccccaatgcatccaacttgaaaattgtaagaatggacaggacagctggatgtgtgactggaggggaggaaatttatcttctttgtgacaaagttcagaaagatgacatccagattcgattttatgaagaggaagaaaatggtggagtctgggaaggatttggagatttttcccccacagatgttcatagacaatttgccattgtcttcaaaactccaaagtataaagatattaatattacaaaaccagcctctgtgtttgtccagcttcggaggaaatctgacttggaaactagtgaaccaaaacctttcctctactatcctgaaatcaaagataaagaagaagtgcagaggaaacgtcagaagctcatgcccaatttttcggatagtttcggcggtggtagtggtgccggagctggaggcggaggcatgtttggtagtggcggtggaggagggggcactggaagtacaggtccagggtatagcttcccacactatggatttcctacttatggtgggattactttccatcctggaactactaaatctaatgctgggatgaagcatggaaccatggacactgaatctaaaaaggaccctgaaggttgtgacaaaagtgatgacaaaaacactgtaaacctctttgggaaagttattgaaaccacagagcaagatcaggagcccagcgaggccaccgttgggaatggtgaggtcactctaacgtatgcaacaggaacaaaagaagagagtgctggagttcaggataacctctttctagagaaggctatgcagcttgcaaagaggcatgccaatgcccttttcgactacgcggtgacaggagacgtgaagatgctgctggccgtccagcgccatctcactgctgtgcaggatgagaatggggacagtgtcttacacttagcaatcatccaccttcattctcaacttgtgagggatctactagaagtcacatctggtttgatttctgatgacattatcaacatgagaaatgatctgtaccagacgcccttgcacttggcagtgatcactaagcaggaagatgtggtggaggatttgctgagggctggggccgacctgagccttctggaccgcttgggtaactctgttttgcacctagctgccaaagaaggacatgataaagttctcagtatcttactcaagcacaaaaaggcagcactacttcttgaccaccccaacggggacggtctgaatgccattcatctagccatgatgagcaatagcctgccatgtttgctgctgctggtggccgctggggctgacgtcaatgctcaggagcagaagtccgggcgcacagcactgcacctggctgtggagcacgacaacatctcattggcaggctgcctgctcctggagggtgatgcccatgtggacagtactacctacgatggaaccacacccctgcatatagcagctgggagagggtccaccaggctggcagctcttctcaaagcagcaggagcagatcccctggtggagaactttgagcctctctatgacctggatgactcttgggaaaatgcaggagaggatgaaggagttgtgcctggaaccacgcctctagatatggccaccagctggcaggtatttgacatattaaatgggaaaccatatgagccagagtttacatctgatgatttactagcacaaggagacatgaaacagctggctgaagatgtgaagctgcagctgtataagttactagaaattcctgatccagacaaaaactgggctactctggcgcagaaattaggtctggggatacttaataatgccttccggctgagtcctgctccttccaaaacacttatggacaactatgaggtctctgggggtacagtcagagagctggtggaggccctgagacaaatgggctacaccgaagcaattgaagtgatccaggcagcctccagcccagtgaagaccacctctcaggcccactcgctgcctctctcgcctgcctccacaaggcagcaaatagacgagctccgagacagtgacagtgtctgcgacagcggcgtggagacatccttccgcaaactcagctttaccgagtctctgaccagtggtgcctcactgctaactctcaacaaaatgccccatgattatgggcaggaaggacctctagaaggcaaaattttg

SEQ ID NO: 118 - protein, 970 aa, Homo sapiens
MAEDDPYLGRPEQMFHLDPSLTHTIFNPEVFQPQMALPTADGPYLQILEQPKQRGFRFRYVCEGPSHGGLPGASSEKNKKSYPQVKICNYVGPAKVIVQLVTNGKNIHLHAHSLVGKHCEDGICTVTAGPKDMVVGFANLGILHVTKKKVFETLEARMTEACIRGHNPGLLVHPDLAYLQAEGGGDRQLGDREKELIRQAALQQTKEMDLSVVRLMFTAFLPDSTGSFTRRLEPVVSDAIYDSKAPNASNLKIVRMDRTAGCVTGGEEIYLLCDKVQKDDIQIRFYEEEENGGVWEGFGDFSPTDVHRQFAIVFKTPKYKDINITKPASVFVQLRRKSDLETSEPKPFLYYPEIKDKEEVQRKRQKLMPNFSDSFGGGSGAGAGGGGMFGSGGGGGGTGSTGPGYSFPHYGFPTYGGITFHPGTTKSNAGMKHGTMDTESKKDPEGCDKSDDKNTVNLFGKVIETTEQDQEPSEATVGNGEVTLTYATGTKEESAGVQDNLFLEKAMQLAKRHANALFDYAVTGDVKMLLAVQRHLTAVQDENGDSVLHLAIIHLHSQLVRDLLEVTSGLISDDIINMRNDLYQTPLHLAVITKQEDVVEDLLRAGADLSLLDRLGNSVLHLAAKEGHDKVLSILLKHKKAALLLDHPNGDGLNAIHLAMMSNSLPCLLLLVAAGADVNAQEQKSGRTALHLAVEHDNISLAGCLLLEGDAHVDSTTYDGTTPLHIAAGRGSTRLAALLKAAGADPLVENFEPLYDLDDSWENAGEDEGVVPGTTPLDMATSWQVFDILNGKPYEPEFTSDDLLAQGDMKQLAEDVKLQLYKLLEIPDPDKNWATLAQKLGLGILNNAFRLSPAPSKTLMDNYEVSGGTVRELVEALRQMGYTEAIEVIQAASSPVKTTSQAHSLPLSPASTRQQIDELRDSDSVCDSGVETSFRKLSFTESLTSGASLLTLNKMPHDYGQEGPLEGKIL

SEQ ID NO: 119 - DNA, 1966 bp, Synthetic construct
  source 1..1966: mol_type = other DNA
atggagagttgctacaacccaggtctggatggtattattgaatatgatgatttcaaattgaactcctccattgtggaacccaaggagccagccccagaaacagctgatggcccctacctggtgatcgtggaacagcctaagcagagaggcttccgatttcgatatggctgtgaaggcccctcccatggaggactgcccggtgcctccagtgagaagggccgaaagacctatcccactgtcaagatctgtaactacgagggaccagccaagatcgaggtggacctggtaacacacagtgacccacctcgtgctcatgcccacagtctggtgggcaagcaatgctcggagctggggatctgcgccgtttctgtggggcccaaggacatgactgcccaatttaacaacctgggtgtcctgcatgtgactaagaagaacatgatggggactatgatacaaaaacttcagaggcagcggctccgctctaggccccagggccttacggaggccgagcagcgggagctggagcaagaggccaaagaactgaagaaggtgatggatctgagtatagtgcggctgcgcttctctgccttccttagagccagtgatggctccttctccctgcccctgaagccagtcatctcccagcccatccatgacagcaaatctccgggggcatcaaacctgaagatttctcgaatggacaagacagcaggctctgtgcggggtggagatgaagtttatctgctttgtgacaaggtgcagaaagatgacattgaggttcggttctatgaggatgatgagaatggatggcaggcctttggggacttctctcccacagatgtgcataaacagtatgccattgtgttccggacacccccctatcacaagatgaagattgagcggcctgtaacagtgtttctgcaactgaaacgcaagcgaggaggggacgtgtctgattccaaacagttcacctattaccctctggtggaagacaaggaagaggtgcagcggaagcggaggaaggccttgcccaccttctcccagcccttcgggggtggctcccacatgggtggaggctctgggggtgcagccgggggctacggaggagctggaggaggtggcagcctcggtttcttcccctcctccctggcctacagcccctaccagtccggcgcgggccccatgggctgctacccgggaggcgggggcggggcgcagatggccgccacggtgcccagcagggactccggggaggaagccgcggagccgagcgccccctccaggaccccccagtgcgagccgcaggccccggagatgctgcagcgagctcgagagtacaacgcgcgcctgttcggcctggcgcagcgcagcgcccgagccctactcgactacggcgtcaccgcggacgcgcgcgcgctgctggcgggacagcgccacctgctgacggcgcaggacgagaacggagacacaccactgcacctagccatcatccacgggcagaccagtgtcattgagcagatagtctatgtcatccaccacgcccaggacctcggcgttgtcaacctcaccaaccacctgcaccagacgcccctgcacctggcggtgatcacggggcagacgagtgtggtgagctttctgctgcgggtaggtgcagacccagctctgctggatcggcatggagactcagccatgcatctggcgctgcgggcaggcgctggtgctcctgagctgctgcgtgcactgcttcagagtggagctcctgctgtgccccagctgttgcatatgcctgactttgagggactgtatccagtacacctggcggtccgagcccgaagccctgagtgcctggatctgctggtggacagtggggctgaagtggaggccactgagcggcaggggggacgaacagccttgcatctagccacagagatggaggagctggggttggtcacccatctggtcaccg

SEQ ID NO: 120 - protein, 655 aa, Synthetic construct
MESCYNPGLDGIIEYDDFKLNSSIVEPKEPAPETADGPYLVIVEQPKQRGFRFRYGCEGPSHGGLPGASSEKGRKTYPTVKICNYEGPAKIEVDLVTHSDPPRAHAHSLVGKQCSELGICAVSVGPKDMTAQFNNLGVLHVTKKNMMGTMIQKLQRQRLRSRPQGLTEAEQRELEQEAKELKKVMDLSIVRLRFSAFLRASDGSFSLPLKPVISQPIHDSKSPGASNLKISRMDKTAGSVRGGDEVYLLCDKVQKDDIEVRFYEDDENGWQAFGDFSPTDVHKQYAIVFRTPPYHKMKIERPVTVFLQLKRKRGGDVSDSKQFTYYPLVEDKEEVQRKRRKALPTFSQPFGGGSHMGGGSGGAAGGYGGAGGGGSLGFFPSSLAYSPYQSGAGPMGCYPGGGGGAQMAATVPSRDSGEEAAEPSAPSRTPQCEPQAPEMLQRAREYNARLFGLAQRSARALLDYGVTADARALLAGQRHLLTAQDENGDTPLHLAIIHGQTSVIEQIVYVIHHAQDLGVVNLTNHLHQTPLHLAVITGQTSVVSFLLRVGADPALLDRHGDSAMHLALRAGAGAPELLRALLQSGAPAVPQLLHMPDFEGLYPVHLAVRARSPECLDLLVDSGAEVEATERQGGRTALHLATEMEELGLVTHLVT

SEQ ID NO: 121 - DNA, 2697 bp, Homo sapiens
  source 1..2697: mol_type = genomic DNA
atggagagttgctacaacccaggtctggatggtattattgaatatgatgatttcaaattgaactcctccattgtggaacccaaggagccagccccagaaacagctgatggcccctacctggtgatcgtggaacagcctaagcagagaggcttccgatttcgatatggctgtgaaggcccctcccatggaggactgcccggtgcctccagtgagaagggccgaaagacctatcccactgtcaagatctgtaactacgagggaccagccaagatcgaggtggacctggtaacacacagtgacccacctcgtgctcatgcccacagtctggtgggcaagcaatgctcggagctggggatctgcgccgtttctgtggggcccaaggacatgactgcccaatttaacaacctgggtgtcctgcatgtgactaagaagaacatgatggggactatgatacaaaaacttcagaggcagcggctccgctctaggccccagggccttacggaggccgagcagcgggagctggagcaagaggccaaagaactgaagaaggtgatggatctgagtatagtgcggctgcgcttctctgccttccttagagccagtgatggctccttctccctgcccctgaagccagtcatctcccagcccatccatgacagcaaatctccgggggcatcaaacctgaagatttctcgaatggacaagacagcaggctctgtgcggggtggagatgaagtttatctgctttgtgacaaggtgcagaaagatgacattgaggttcggttctatgaggatgatgagaatggatggcaggcctttggggacttctctcccacagatgtgcataaacagtatgccattgtgttccggacacccccctatcacaagatgaagattgagcggcctgtaacagtgtttctgcaactgaaacgcaagcgaggaggggacgtgtctgattccaaacagttcacctattaccctctggtggaagacaaggaagaggtgcagcggaagcggaggaaggccttgcccaccttctcccagcccttcgggggtggctcccacatgggtggaggctctgggggtgcagccgggggctacggaggagctggaggaggtggcagcctcggtttcttcccctcctccctggcctacagcccctaccagtccggcgcgggccccatgggctgctacccgggaggcgggggcggggcgcagatggccgccacggtgcccagcagggactccggggaggaagccgcggagccgagcgccccctccaggaccccccagtgcgagccgcaggccccggagatgctgcagcgagctcgagagtacaacgcgcgcctgttcggcctggcgcagcgcagcgcccgagccctactcgactacggcgtcaccgcggacgcgcgcgcgctgctggcgggacagcgccacctgctgacggcgcaggacgagaacggagacacaccactgcacctagccatcatccacgggcagaccagtgtcattgagcagatagtctatgtcatccaccacgcccaggacctcggcgttgtcaacctcaccaaccacctgcaccagacgcccctgcacctggcggtgatcacggggcagacgagtgtggtgagctttctgctgcgggtaggtgcagacccagctctgctggatcggcatggagactcagccatgcatctggcgctgcgggcaggcgctggtgctcctgagctgctgcgtgcactgcttcagagtggagctcctgctgtgccccagctgttgcatatgcctgactttgagggactgtatccagtacacctggcggtccgagcccgaagccctgagtgcctggatctgctggtggacagtggggctgaagtggaggccactgagcggcaggggggacgaacagccttgcatctagccacagagatggaggagctggggttggtcacccatctggtcaccaagctccgggccaacgtgaacgctcgcacctttgcgggaaacacacccctgcacctggcagctggactggggtacccgaccctcacccgcctccttctgaaggctggtgctgacatccatgctgaaaacgaggagcccctgtgcccactgccttcaccccctacctctgatagcgactcggactctgaagggcctgagaaggacacccgaagcagcttccggggccacacgcctcttgacctcacttgcagcaccaaggtgaagaccttgctgctaaatgctgctcagaacaccatggagccacccctgaccccgcccagcccagcagggccgggactgtcacttggtgatacagctctgcagaacctggagcagctgctagacgggccagaagcccagggcagctgggcagagctggcagagcgtctggggctgcgcagcctggtagacacgtaccgacagacaacctcacccagtggcagcctcctgcgcagctacgagctggctggcggggacctggcaggtctactggaggccctgtctgacatgggcctagaggagggagtgaggctgctgaggggtccagaaacccgagacaagctgcccagcacagaggtgaaggaagacagtgcgtacgggagccagtcagtggagcaggaggcagagaagctgggcccaccccctgagccaccaggagggctctgccacgggcacccccagcctcaggtgcac

SEQ ID NO: 122 - protein, 899 aa, Homo sapiens
MESCYNPGLDGIIEYDDFKLNSSIVEPKEPAPETADGPYLVIVEQPKQRGFRFRYGCEGPSHGGLPGASSEKGRKTYPTVKICNYEGPAKIEVDLVTHSDPPRAHAHSLVGKQCSELGICAVSVGPKDMTAQFNNLGVLHVTKKNMMGTMIQKLQRQRLRSRPQGLTEAEQRELEQEAKELKKVMDLSIVRLRFSAFLRASDGSFSLPLKPVISQPIHDSKSPGASNLKISRMDKTAGSVRGGDEVYLLCDKVQKDDIEVRFYEDDENGWQAFGDFSPTDVHKQYAIVFRTPPYHKMKIERPVTVFLQLKRKRGGDVSDSKQFTYYPLVEDKEEVQRKRRKALPTFSQPFGGGSHMGGGSGGAAGGYGGAGGGGSLGFFPSSLAYSPYQSGAGPMGCYPGGGGGAQMAATVPSRDSGEEAAEPSAPSRTPQCEPQAPEMLQRAREYNARLFGLAQRSARALLDYGVTADARALLAGQRHLLTAQDENGDTPLHLAIIHGQTSVIEQIVYVIHHAQDLGVVNLTNHLHQTPLHLAVITGQTSVVSFLLRVGADPALLDRHGDSAMHLALRAGAGAPELLRALLQSGAPAVPQLLHMPDFEGLYPVHLAVRARSPECLDLLVDSGAEVEATERQGGRTALHLATEMEELGLVTHLVTKLRANVNARTFAGNTPLHLAAGLGYPTLTRLLLKAGADIHAENEEPLCPLPSPPTSDSDSDSEGPEKDTRSSFRGHTPLDLTCSTKVKTLLLNAAQNTMEPPLTPPSPAGPGLSLGDTALQNLEQLLDGPEAQGSWAELAERLGLRSLVDTYRQTTSPSGSLLRSYELAGGDLAGLLEALSDMGLEEGVRLLRGPETRDKLPSTEVKEDSAYGSQSVEQEAEKLGPPPEPPGGLCHGHPQPQVH

SEQ ID NO: 123 - DNA, 567 bp, Synthetic construct
  source 1..567: mol_type = other DNA
atgactgagtataagctcgttgtagtgggtgcaggaggtgttggcaaatccgccttgactatccaattgattcaaaatcatttcgtcgacgagtacgaccctaccatagaggattcctatcgcaagcaggtagtaatcgacggcgagacatgccttcttgatattctggatacggcgggacacgaggaatactctgcaatgcgcgaccagtatatgcgaaccggtgagggttttttgtgtgtttttgcgataaataattccaagtcatttgcggacataaatctgtatcgggagcagatcaagcgcgttaaggattcagacgatgtgcctatggtcctcgtggggaataagtgtgaccttcctacccgaacagtcgacacaaaacaggcccatgaactggctaaaagctacgggataccgtttattgaaacatcagcaaaaactagacagggagttgaagacgcgttctatacccttgtcagggagatacgacaatacagaatgaaaaaactcaatagttccgacgatggcactcaaggttgtatgggtttgccgtgtgtggttatg

SEQ ID NO: 124 - protein, 189 aa, Synthetic construct [sequence omitted]

SEQ ID NO: 125 - DNA, 567 bp, Synthetic construct
  source 1..567: mol_type = other DNA
atgactgagtataagctcgttgtagtgggtgcaggagatgttggcaaatccgccttgactatccaattgattcaaaatcatttcgtcgacgagtacgaccctaccatagaggattcctatcgcaagcaggtagtaatcgacggcgagacatgccttcttgatattctggatacggcgggacaggaggaatactctgcaatgcgcgaccagtatatgcgaaccggtgagggttttttgtgtgtttttgcgataaataattccaagtcatttgcggacataaatctgtatcgggagcagatcaagcgcgttaaggattcagacgatgtgcctatggtcctcgtggggaataagtgtgaccttcctacccgaacagtcgacacaaaacaggcccatgaactggctaaaagctacgggataccgtttattgaaacatcagcaaaaactagacagggagttgaagacgcgttctatacccttgtcagggagatacgacaatacagaatgaaaaaactcaatagttccgacgatggcactcaaggttgtatgggtttgccgtgtgtggttatg

SEQ ID NO: 126 - protein, 189 aa, Synthetic construct
MTEYKLVVVGAGDVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 127 - DNA, 567 bp, Homo sapiens
  source 1..567: mol_type = genomic DNA
atgactgagtataagctcgttgtagtgggtgcaggaggtgttggcaaatccgccttgactatccaattgattcaaaatcatttcgtcgacgagtacgaccctaccatagaggattcctatcgcaagcaggtagtaatcgacggcgagacatgccttcttgatattctggatacggcgggacaggaggaatactctgcaatgcgcgaccagtatatgcgaaccggtgagggttttttgtgtgtttttgcgataaataattccaagtcatttgcggacataaatctgtatcgggagcagatcaagcgcgttaaggattcagacgatgtgcctatggtcctcgtggggaataagtgtgaccttcctacccgaacagtcgacacaaaacaggcccatgaactggctaaaagctacgggataccgtttattgaaacatcagcaaaaactagacagggagttgaagacgcgttctatacccttgtcagggagatacgacaatacagaatgaaaaaactcaatagttccgacgatggcactcaaggttgtatgggtttgccgtgtgtggttatg

SEQ ID NO: 128 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 129 - DNA, 690 bp, Synthetic construct
  source 1..690: mol_type = other DNA
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgctttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtgg

SEQ ID NO: 130 - protein, 230 aa, Synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQW

SEQ ID NO: 131 - DNA, 864 bp, Homo sapiens
  source 1..864: mol_type = genomic DNA
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgctttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagccgacggccctcggagtgcccagccactgaggcctgaggatggacactgctcttggcccctc

SEQ ID NO: 132 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 133 - DNA, 3873 bp, Synthetic construct
  source 1..3873: mol_type = other DNA
atggcgggcgccgcgtccccttgcgccaacggctgcgggcccggcgcgccctcggacgccgaggtgctgcacctctgccgcagcctcgaggtgggcaccgtcatgactttgttctactccaagaagtcgcagcgacccaagcggaagaccttccaggtcaagctggagacgcgccagatcacgtggagccggggcgccgacaagatcgagggggccattgacattcgtgaaattaaggagatccgcccagggaagacctcacgggactttgatcgctatcaagaggacccagctttccggccggaccagtcacattgctttgtcattctctatggaatggaatttcgcctgaaaacgctgagcctgcaagccacatctgaggatgaagtgaacatgtggatcaagggcttaacttggctgatggaggatacattgcaggcacccacacccctgcagattgagaggtggctccggaagcagttttactcagtggatcggaatcgtgaggatcgtatatcagccaaggacctgaagaacatgctgtcccaggtcaactaccgggtccccaacatgcgcttcctccgagagcggctgacggacctggagcagcgcagcggggacatcacctacgggcagtttgctcagctgtaccgcagcctcatgtacagcgcccagaagacgatggacctccccttcttggaagccagtactctgagggctggggagcggccggagctttgccgagtgtcccttcctgagttccagcagttccttcttgactaccagggggagctgtgggctgttgatcgcctccaggtgcaggagttcatgctcagcttcctccgagaccccttacgagagatcgaggagccatacttcttcctggatgagtttgtcaccttcctgttctccaaagagaacagtgtgtggaactcgcagctggatgcagtatgcccggacaccatgaacaaccctctttcccactactggatctcctcctcgcacaacacgtacctgaccggggaccagttctccagtgagtcctccttggaagcctatgctcgctgcctgcggatgggctgtcgctgcattgagttggactgctgggacggcccggatgggatgccagttatttaccatgggcacacccttaccaccaagatcaagttctcagatgtcctgcacaccatcaaggagcatgcctttgtggcctcagagtacccagtcatcctgtccattgaggaccactgcagcattgcccagcagagaaacatggcccaatacttcaagaaggtgctgggggacacactcctcaccaagcccgtggagatctctgccgacgggctcccctcacccaaccagcttaagaggaagatcctcatcaagcacaagaagctggctgagggcagtgcctacgaggaggtgcctacatccatgatgtactctgagaacgacatcagcaactctatcaagaatggcatcctctacctggaggaccctgtgaaccacgaatggtatccccactactttgttctgaccagcagcaagatctactactctgaggagaccagcagtgaccagggcaacgaggatgaggaggagcccaaggaggtcagcagcagcacagagctgcactccaatgagaagtggttccatgggaagctaggggcagggcgtgacgggcgtcacatcgctgagcgcctgcttactgagtactgcatcgagaccggagcccctgacggctccttcctcgtgcgagagagtgagaccttcgtgggcgactacacgctctctttctggcggaacgggaaagtccagcactgccgtatccactcccggcaagatgctgggacccccaagttcttcttgacagacaacctcgtctttgactccctctatgacctcatcacgcactaccagcaggtgcccctgcgctgtaatgagtttgagatgcgactttcagagcctgtcccacagaccaacgcccacgagagcaaagagtggtaccacgcgagcctgaccagagcacaggctgagcacatgctaatgcgcgtccctcgtgatggggccttcctggtgcggaagcggaatgaacccaactcatatgccatctctttccgggctgagggcaagatcaagcattgccgtgtccagcaagagggccagacagtgatgctagggaactcggagttcgacagccttgttgacctcatcagctactatgagaaacacccgctataccgcaagatgaagctgcgctatcccatcaacgaggaggcactggagaagattggcacagctgagcctgactacggggccctgtatgagggacgcaaccctggcttctatgtagaggcaaaccctatgccaactttcaagtgtgcagtcaaagccctctttgactacaaggcccagagggaggacgagctgaccttcaccaagagcgccatcatccagaatgtggagaagcaagagggaggctggtggcgaggggactacggagggaagaagcagctgtggttcccatcaaactacgtggaagagatggtcaaccccgtggccctggagccggagagggagcacttggacgagaacagccccctaggggacttgctgcggggggtcttggatgtgccggcttgtcagattgccatccgtcctgagggcaagaacaaccggctcttcgtcttctccatcagcatggcgtcggtggcccactggtccctggatgttgctgccgactcacaggaggagctgcaggactgggtgaaaaagatccgtgaagtggcccagacagcagacgccaggctcactgaagggaagataatggaacggaggaagaagattgccctggagctctctgaacttgtcgtctactgccggcctgttccctttgatgaagagaagattggcacagaacgtgcttgctaccgggacatgtcatccttcccggaaaccaaggctgagaaatacgtgaacaaggccaaaggcaagaagttccttcagtacaatcgactgcagctctcccgcatctaccccaagggccagcgactggattcctccaactacgatcctttgcccatgtggatctgtggcagtcagcttgtggccctcaacttccagacccctgacaagcctatgcagatgaaccaggccctcttcatgacgggcaggcactgtggctacgtgctgcagccaagcaccatgcgggatgaggccttcgacccctttgacaagagcagcctccgcgggctggagccatgtgccatctctattgaggtgctgggggcccgacatctgccaaagaatggccgaggcattgtgtgtccttttgtggagattgaggtggctggagctgagtatgacagcaccaagcagaagacagagtttgtggtggacaatggactcaaccctgtatggccagccaagcccttccacttccagatcagtaaccctgaatttgcctttctgcgcttcgtggtgtatgaggaagacatgtttagtgaccagaatttcctggctcaggctactttcccagtaaaaggcctgaagacaggatacagagcagtgcctttgaagaacaactacagtgaggacctggagttggcctccctgctgatcaagattgacattttccctgccaagcaggagaatggtgacctcagtcccttcagtggtacgtccctgcgggagcggggctcagatgcctcaggccagctgtttcatggccgagcccgggaaggctcctttgaatcccgctaccagcagccgtttgaggacttccgcatctcccaggagcatctcgcagaccattttgacagtcgagaacgaagggccccaagaaggactcgggtcaatggagacaaccgcctc

SEQ ID NO: 134 - protein, 1291 aa, Synthetic construct
MAGAASPCANGCGPGAPSDAEVLHLCRSLEVGTVMTLFYSKKSQRPKRKTFQVKLETRQITWSRGADKIEGAIDIREIKEIRPGKTSRDFDRYQEDPAFRPDQSHCFVILYGMEFRLKTLSLQATSEDEVNMWIKGLTWLMEDTLQAPTPLQIERWLRKQFYSVDRNREDRISAKDLKNMLSQVNYRVPNMRFLRERLTDLEQRSGDITYGQFAQLYRSLMYSAQKTMDLPFLEASTLRAGERPELCRVSLPEFQQFLLDYQGELWAVDRLQVQEFMLSFLRDPLREIEEPYFFLDEFVTFLFSKENSVWNSQLDAVCPDTMNNPLSHYWISSSHNTYLTGDQFSSESSLEAYARCLRMGCRCIELDCWDGPDGMPVIYHGHTLTTKIKFSDVLHTIKEHAFVASEYPVILSIEDHCSIAQQRNMAQYFKKVLGDTLLTKPVEISADGLPSPNQLKRKILIKHKKLAEGSAYEEVPTSMMYSENDISNSIKNGILYLEDPVNHEWYPHYFVLTSSKIYYSEETSSDQGNEDEEEPKEVSSSTELHSNEKWFHGKLGAGRDGRHIAERLLTEYCIETGAPDGSFLVRESETFVGDYTLSFWRNGKVQHCRIHSRQDAGTPKFFLTDNLVFDSLYDLITHYQQVPLRCNEFEMRLSEPVPQTNAHESKEWYHASLTRAQAEHMLMRVPRDGAFLVRKRNEPNSYAISFRAEGKIKHCRVQQEGQTVMLGNSEFDSLVDLISYYEKHPLYRKMKLRYPINEEALEKIGTAEPDYGALYEGRNPGFYVEANPMPTFKCAVKALFDYKAQREDELTFTKSAIIQNVEKQEGGWWRGDYGGKKQLWFPSNYVEEMVNPVALEPEREHLDENSPLGDLLRGVLDVPACQIAIRPEGKNNRLFVFSISMASVAHWSLDVAADSQEELQDWVKKIREVAQTADARLTEGKIMERRKKIALELSELVVYCRPVPFDEEKIGTERACYRDMSSFPETKAEKYVNKAKGKKFLQYNRLQLSRIYPKGQRLDSSNYDPLPMWICGSQLVALNFQTPDKPMQMNQALFMTGRHCGYVLQPSTMRDEAFDPFDKSSLRGLEPCAISIEVLGARHLPKNGRGIVCPFVEIEVAGAEYDSTKQKTEFVVDNGLNPVWPAKPFHFQISNPEFAFLRFVVYEEDMFSDQNFLAQATFPVKGLKTGYRAVPLKNNYSEDLELASLLIKIDIFPAKQENGDLSPFSGTSLRERGSDASGQLFHGRAREGSFESRYQQPFEDFRISQEHLADHFDSRERRAPRRTRVNGDNRL

SEQ ID NO: 135 - DNA, 3873 bp, Synthetic construct
  source 1..3873: mol_type = other DNA
atggcgggcgccgcgtccccttgcgccaacggctgcgggcccggcgcgccctcggacgccgaggtgctgcacctctgccgcagcctcgaggtgggcaccgtcatgactttgttctactccaagaagtcgcagcgacccgagcggaagaccttccaggtcaagctggagacgcgccagatcacgtggagccggggcgccgacaagatcgagggggccattgacattcgtgaaattaaggagatccgcccagggaagacctcacgggactttgatcgctatcaagaggacccagctttccggccggaccagtcacattgctttgtcattctctatggaatggaatttcgcctgaaaacgctgagcctgcaagccacatctgaggatgaagtgaacatgtggatcaagggcttaacttggctgatggaggatacattgcaggcacccacacccctgcagattgagaggtggctccggaagcagttttactcagtggatcggaatcgtgaggatcgtatatcagccaaggacctgaagaacatgctgtcccaggtcaactaccgggtccccaacatgcgcttcctccgagagcggctgacggacctggagcagcgcagcggggacatcacctacgggcagtttgctcagctgtaccgcagcctcatgtacagcgcccagaagacgatggacctccccttcttggaagccagtactctgagggctggggagcggccggagctttgccgagtgtcccttcctgagttccagcagttccttcttgactaccagggggagctgtgggctgttgatcgcctccaggtgcaggagttcatgctcagcttcctccgagaccccttacgagagatcgaggagccatacttcttcctggatgagtttgtcaccttcctgttctccaaagagaacagtgtgtggaactcgcagctggatgcagtatgcccggacaccatgaacaaccctctttcccactactggatctcctcctcgcacaacacgtacctgaccggggaccagttctccagtgagtcctccttggaagcctatgctcgctgcctgcggatgggctgtcgctgcattgagttggactgctgggacggcccggatgggatgccagttatttaccatgggcacacccttaccaccaagatcaagttctcagatgtcctgcacaccatcaaggagcatgcctttgtggcctcagagtacccagtcatcctgtccattgaggaccactgcagcattgcccagcagagaaacatggcccaatacttcaagaaggtgctgggggacacactcctcaccaagcccgtggagatctctgccgacgggctcccctcacccaaccagcttaagaggaagatcctcatcaagcacaagaagctggctgagggcagtgcctacgaggaggtgcctacatccatgatgtactctgagaacgacatcagcaactctatcaagaatggcatcctctacctggaggaccctgtgaaccacgaatggtatccccactactttgttctgaccagcagcaagatctactactttgaggagaccagcagtgaccagggcaacgaggatgaggaggagcccaaggaggtcagcagcagcacagagctgcactccaatgagaagtggttccatgggaagctaggggcagggcgtgacgggcgtcacatcgctgagcgcctgcttactgagtactgcatcgagaccggagcccctgacggctccttcctcgtgcgagagagtgagaccttcgtgggcgactacacgctctctttctggcggaacgggaaagtccagcactgccgtatccactcccggcaagatgctgggacccccaagttcttcttgacagacaacctcgtctttgactccctctatgacctcatcacgcactaccagcaggtgcccctgcgctgtaatgagtttgagatgcgactttcagagcctgtcccacagaccaacgcccacgagagcaaagagtggtaccacgcgagcctgaccagagcacaggctgagcacatgctaatgcgcgtccctcgtgatggggccttcctggtgcggaagcggaatgaacccaactcatatgccatctctttccgggctgagggcaagatcaagcattgccgtgtccagcaagagggccagacagtgatgctagggaactcggagttcgacagccttgttgacctcatcagctactatgagaaacacccgctataccgcaagatgaagctgcgctatcccatcaacgaggaggcactggagaagattggcacagctgagcctgactacggggccctgtatgagggacgcaaccctggcttctatgtagaggcaaaccctatgccaactttcaagtgtgcagtcaaagccctctttgactacaaggcccagagggaggacgagctgaccttcaccaagagcgccatcatccagaatgtggagaagcaagagggaggctggtggcgaggggactacggagggaagaagcagctgtggttcccatcaaactacgtggaagagatggtcaaccccgtggccctggagccggagagggagcacttggacgagaacagccccctaggggacttgctgcggggggtcttggatgtgccggcttgtcagattgccatccgtcctgagggcaagaacaaccggctcttcgtcttctccatcagcatggcgtcggtggcccactggtccctggatgttgctgccgactcacaggaggagctgcaggactgggtgaaaaagatccgtgaagtggcccagacagcagacgccaggctcactgaagggaagataatggaacggaggaagaagattgccctggagctctctgaacttgtcgtctactgccggcctgttccctttgatgaagagaagattggcacagaacgtgcttgctaccgggacatgtcatccttcccggaaaccaaggctgagaaatacgtgaacaaggccaaaggcaagaagttccttcagtacaatcgactgcagctctcccgcatctaccccaagggccagcgactggattcctccaactacgatcctttgcccatgtggatctgtggcagtcagcttgtggccctcaacttccagacccctgacaagcctatgcagatgaaccaggccctcttcatgacgggcaggcactgtggctacgtgctgcagccaagcaccatgcgggatgaggccttcgacccctttgacaagagcagcctccgcgggctggagccatgtgccatctctattgaggtgctgggggcccgacatctgccaaagaatggccgaggcattgtgtgtccttttgtggagattgaggtggctggagctgagtatgacagcaccaagcagaagacagagtttgtggtggacaatggactcaaccctgtatggccagccaagcccttccacttccagatcagtaaccctgaatttgcctttctgcgcttcgtggtgtatgaggaagacatgtttagtgaccagaatttcctggctcaggctactttcccagtaaaaggcctgaagacaggatacagagcagtgcctttgaagaacaactacagtgaggacctggagttggcctccctgctgatcaagattgacattttccctgccaagcaggagaatggtgacctcagtcccttcagtggtacgtccctgcgggagcggggctcagatgcctcaggccagctgtttcatggccgagcccgggaaggctcctttgaatcccgctaccagcagccgtttgaggacttccgcatctcccaggagcatctcgcagaccattttgacagtcgagaacgaagggccccaagaaggactcgggtcaatggagacaaccgcctc

SEQ ID NO: 136 - protein, 1291 aa, Synthetic construct
MAGAASPCANGCGPGAPSDAEVLHLCRSLEVGTVMTLFYSKKSQRPERKTFQVKLETRQITWSRGADKIEGAIDIREIKEIRPGKTSRDFDRYQEDPAFRPDQSHCFVILYGMEFRLKTLSLQATSEDEVNMWIKGLTWLMEDTLQAPTPLQIERWLRKQFYSVDRNREDRISAKDLKNMLSQVNYRVPNMRFLRERLTDLEQRSGDITYGQFAQLYRSLMYSAQKTMDLPFLEASTLRAGERPELCRVSLPEFQQFLLDYQGELWAVDRLQVQEFMLSFLRDPLREIEEPYFFLDEFVTFLFSKENSVWNSQLDAVCPDTMNNPLSHYWISSSHNTYLTGDQFSSESSLEAYARCLRMGCRCIELDCWDGPDGMPVIYHGHTLTTKIKFSDVLHTIKEHAFVASEYPVILSIEDHCSIAQQRNMAQYFKKVLGDTLLTKPVEISADGLPSPNQLKRKILIKHKKLAEGSAYEEVPTSMMYSENDISNSIKNGILYLEDPVNHEWYPHYFVLTSSKIYYFEETSSDQGNEDEEEPKEVSSSTELHSNEKWFHGKLGAGRDGRHIAERLLTEYCIETGAPDGSFLVRESETFVGDYTLSFWRNGKVQHCRIHSRQDAGTPKFFLTDNLVFDSLYDLITHYQQVPLRCNEFEMRLSEPVPQTNAHESKEWYHASLTRAQAEHMLMRVPRDGAFLVRKRNEPNSYAISFRAEGKIKHCRVQQEGQTVMLGNSEFDSLVDLISYYEKHPLYRKMKLRYPINEEALEKIGTAEPDYGALYEGRNPGFYVEANPMPTFKCAVKALFDYKAQREDELTFTKSAIIQNVEKQEGGWWRGDYGGKKQLWFPSNYVEEMVNPVALEPEREHLDENSPLGDLLRGVLDVPACQIAIRPEGKNNRLFVFSISMASVAHWSLDVAADSQEELQDWVKKIREVAQTADARLTEGKIMERRKKIALELSELVVYCRPVPFDEEKIGTERACYRDMSSFPETKAEKYVNKAKGKKFLQYNRLQLSRIYPKGQRLDSSNYDPLPMWICGSQLVALNFQTPDKPMQMNQALFMTGRHCGYVLQPSTMRDEAFDPFDKSSLRGLEPCAISIEVLGARHLPKNGRGIVCPFVEIEVAGAEYDSTKQKTEFVVDNGLNPVWPAKPFHFQISNPEFAFLRFVVYEEDMFSDQNFLAQATFPVKGLKTGYRAVPLKNNYSEDLELASLLIKIDIFPAKQENGDLSPFSGTSLRERGSDASGQLFHGRAREGSFESRYQQPFEDFRISQEHLADHFDSRERRAPRRTRVNGDNRL

SEQ ID NO: 137 - DNA, 3873 bp, Synthetic construct
  source 1..3873: mol_type = other DNA
atggcgggcgccgcgtccccttgcgccaacggctgcgggcccggcgcgccctcggacgccgaggtgctgcacctctgccgcagcctcgaggtgggcaccgtcatgactttgttctactccaagaagtcgcagcgacccgagcggaagaccttccaggtcaagctggagacgcgccagatcacgtggagccggggcgccgacaagatcgagggggccattgacattcgtgaaattaaggagatccgcccagggaagacctcacgggactttgatcgctatcaagaggacccagctttccggccggaccagtcacattgctttgtcattctctatggaatggaatttcgcctgaaaacgctgagcctgcaagccacatctgaggatgaagtgaacatgtggatcaagggcttaacttggctgatggaggatacattgcaggcacccacacccctgcagattgagaggtggctccggaagcagttttactcagtggatcggaatcgtgaggatcgtatatcagccaaggacctgaagaacatgctgtcccaggtcaactaccgggtccccaacatgcgcttcctccgagagcggctgacggacctggagcagcgcagcggggacatcacctacgggcagtttgctcagctgtaccgcagcctcatgtacagcgcccagaagacgatggacctccccttcttggaagccagtactctgagggctggggagcggccggagctttgccgagtgtcccttcctgagttccagcagttccttcttgactaccagggggagctgtgggctgttgatcgcctccaggtgcaggagttcatgctcagcttcctccgagaccccttacgagagatcgaggagccatacttcttcctggatgagtttgtcaccttcctgttctccaaagagaacagtgtgtggaactcgcagctggatgcagtatgcccggacaccatgaacaaccctctttcccactactggatctcctcctcgcacaacacgtacctgaccggggaccagttctccagtgagtcctccttggaagcctatgctcgctgcctgcggatgggctgtcgctgcattgagttggactgctgggacggcccggatgggatgccagttatttaccatgggcacacccttaccaccaagatcaagttctcagatgtcctgcacaccatcaaggagcatgcctttgtggcctcagagtacccagtcatcctgtccattgaggaccactgcagcattgcccagcagagaaacatggcccaatacttcaagaaggtgctgggggacacactcctcaccaagcccgtggagatctctgccgacgggctcccctcacccaaccagcttaagaggaagatcctcatcaagcacaagaagctggctgagggcagtgcctacgaggaggtgcctacatccatgatgtactctgagaacgacatcagcaactctatcaagaatggcatcctctacctggaggaccctgtgaaccacgaatggtatccccactactttgttctgaccagcagcaagatctactactctgaggagaccagcagtgaccagggcaacgaggatgaggaggagcccaaggaggtcagcagcagcacagagctgcactccaatgagaagtggttccatgggaagctaggggcagggcgtgacgggcgtcacatcgctgagcgcctgcttactgagtactgcatcgagaccggagcccctgacggctccttcctcgtgcgagagagtgagaccttcgtgggcgactacacgctctctttctggcggaacgggaaagtccagcactgccgtatccactcccggcaagatgctgggacccccaagttcttcttgacagacaacctcgtctttgactccctctatgacctcatcacgcactaccagcaggtgcccctgcgctgtaatgagtttgagatgcgactttcagagcctgtcccacagaccaacgcccacgagagcaaagagtggtaccacgcgagcctgaccagagcacaggctgagcacatgctaatgcgcgtccctcgtgatggggccttcctggtgcggaagcggaatgaacccaactcatatgccatctctttccgggctgagggcaagatcaagcattgccgtgtccagcaagagggccagacagtgatgctagggaactcggagttcgacagccttgttgacctcatcagctactatgagaaacacccgctataccgcaagatgaagctgcgctatcccatcaacgaggaggcactggagaagattggcacagctgagcctgactacggggccctgtatgagggacgcaaccctggcttctatgtagaggcaaaccctatgccaactttcaagtgtgcagtcaaagccctctttgactacaaggcccagagggaggacgagctgaccttcaccaagagcgccatcatccagaatgtggagaagcaagagggaggctggtggcgaggggactacggagggaagaagcagctgtggttcccatcaaactacgtggaagagatggtcaaccccgtggccctggagccggagagggagcacttggacgagaacagccccctaggggacttgctgcggggggtcttggatgtgccggcttgtcagattgccatccgtcctgagggcaagaacaaccggctcttcgtcttctccatcagcatggcgtcggtggcccactggtccctggatgttgctgccgactcacaggaggagctgcaggactgggtgaaaaagatccgtgaagtggcccagacagcagacgccaggctcactgaagggaagataatggaacggaggaagaagattgccctggagctctctgaacttgtcgtctactgccggcctgttccctttgatgaagagaagattggcacagaacgtgcttgctaccgggacatgtcatccttcccggaaaccaaggctgagaaatacgtgaacaaggccaaaggcaagaagttccttcagtacaatcgactgcagctctcccgcatctaccccaagggccagcgactggattcctccaactacgatcctttgcccatgtggatctgtggcagtcagcttgtggccctcaacttccagacccctgacaagcctatgcagatgaaccaggccctcttcatgacgggcaggcactgtggctacgtgctgcagccaagcaccatgcgggatgaggccttcgacccctttgacaagagcagcctccgcgggctggagccatgtgccatctctattgaggtgctgggggcccgacatctgccaaagaatggccgaggcattgtgtgtccttttgtggagattgaggtggctggagctgagtatgacagcaccaagcagaagacagagtttgtggtggacaatggactcaaccctgtatggccagccaagcccttccacttccagatcagtaaccctgaatttgcctttctgcgcttcgtggtgtataaggaagacatgtttagtgaccagaatttcctggctcaggctactttcccagtaaaaggcctgaagacaggatacagagcagtgcctttgaagaacaactacagtgaggacctggagttggcctccctgctgatcaagattgacattttccctgccaagcaggagaatggtgacctcagtcccttcagtggtacgtccctgcgggagcggggctcagatgcctcaggccagctgtttcatggccgagcccgggaaggctcctttgaatcccgctaccagcagccgtttgaggacttccgcatctcccaggagcatctcgcagaccattttgacagtcgagaacgaagggccccaagaaggactcgggtcaatggagacaaccgcctc

SEQ ID NO: 138 - protein, 1291 aa, Synthetic construct
MAGAASPCANGCGPGAPSDAEVLHLCRSLEVGTVMTLFYSKKSQRPERKTFQVKLETRQITWSRGADKIEGAIDIREIKEIRPGKTSRDFDRYQEDPAFRPDQSHCFVILYGMEFRLKTLSLQATSEDEVNMWIKGLTWLMEDTLQAPTPLQIERWLRKQFYSVDRNREDRISAKDLKNMLSQVNYRVPNMRFLRERLTDLEQRSGDITYGQFAQLYRSLMYSAQKTMDLPFLEASTLRAGERPELCRVSLPEFQQFLLDYQGELWAVDRLQVQEFMLSFLRDPLREIEEPYFFLDEFVTFLFSKENSVWNSQLDAVCPDTMNNPLSHYWISSSHNTYLTGDQFSSESSLEAYARCLRMGCRCIELDCWDGPDGMPVIYHGHTLTTKIKFSDVLHTIKEHAFVASEYPVILSIEDHCSIAQQRNMAQYFKKVLGDTLLTKPVEISADGLPSPNQLKRKILIKHKKLAEGSAYEEVPTSMMYSENDISNSIKNGILYLEDPVNHEWYPHYFVLTSSKIYYSEETSSDQGNEDEEEPKEVSSSTELHSNEKWFHGKLGAGRDGRHIAERLLTEYCIETGAPDGSFLVRESETFVGDYTLSFWRNGKVQHCRIHSRQDAGTPKFFLTDNLVFDSLYDLITHYQQVPLRCNEFEMRLSEPVPQTNAHESKEWYHASLTRAQAEHMLMRVPRDGAFLVRKRNEPNSYAISFRAEGKIKHCRVQQEGQTVMLGNSEFDSLVDLISYYEKHPLYRKMKLRYPINEEALEKIGTAEPDYGALYEGRNPGFYVEANPMPTFKCAVKALFDYKAQREDELTFTKSAIIQNVEKQEGGWWRGDYGGKKQLWFPSNYVEEMVNPVALEPEREHLDENSPLGDLLRGVLDVPACQIAIRPEGKNNRLFVFSISMASVAHWSLDVAADSQEELQDWVKKIREVAQTADARLTEGKIMERRKKIALELSELVVYCRPVPFDEEKIGTERACYRDMSSFPETKAEKYVNKAKGKKFLQYNRLQLSRIYPKGQRLDSSNYDPLPMWICGSQLVALNFQTPDKPMQMNQALFMTGRHCGYVLQPSTMRDEAFDPFDKSSLRGLEPCAISIEVLGARHLPKNGRGIVCPFVEIEVAGAEYDSTKQKTEFVVDNGLNPVWPAKPFHFQISNPEFAFLRFVVYKEDMFSDQNFLAQATFPVKGLKTGYRAVPLKNNYSEDLELASLLIKIDIFPAKQENGDLSPFSGTSLRERGSDASGQLFHGRAREGSFESRYQQPFEDFRISQEHLADHFDSRERRAPRRTRVNGDNRL

SEQ ID NO: 139 - DNA, 3873 bp, Synthetic construct
  source 1..3873: mol_type = other DNA
atggcgggcgccgcgtccccttgcgccaacggctgcgggcccggcgcgccctcggacgccgaggtgctgcacctctgccgcagcctcgaggtgggcaccgtcatgactttgttctactccaagaagtcgcagcgacccgagcggaagaccttccaggtcaagctggagacgcgccagatcacgtggagccggggcgccgacaagatcgagggggccattgacattcgtgaaattaaggagatccgcccagggaagacctcacgggactttgatcgctatcaagaggacccagctttccggccggaccagtcacattgctttgtcattctctatggaatggaatttcgcctgaaaacgctgagcctgcaagccacatctgaggatgaagtgaacatgtggatcaagggcttaacttggctgatggaggatacattgcaggcacccacacccctgcagattgagaggtggctccggaagcagttttactcagtggatcggaatcgtgaggatcgtatatcagccaaggacctgaagaacatgctgtcccaggtcaactaccgggtccccaacatgcgcttcctccgagagcggctgacggacctggagcagcgcagcggggacatcacctacgggcagtttgctcagctgtaccgcagcctcatgtacagcgcccagaagacgatggacctccccttcttggaagccagtactctgagggctggggagcggccggagctttgccgagtgtcccttcctgagttccagcagttccttcttgactaccagggggagctgtgggctgttgatcgcctccaggtgcaggagttcatgctcagcttcctccgagaccccttacgagagatcgaggagccatacttcttcctggatgagtttgtcaccttcctgttctccaaagagaacagtgtgtggaactcgcagctggatgcagtatgcccggacaccatgaacaaccctctttcccactactggatctcctcctcgcacaacacgtacctgaccggggaccagttctccagtgagtcctccttggaagcctatgctcgctgcctgcggatgggctgtcgctgcattgagttggactgctgggacggcccggatgggatgccagttatttaccatgggcacacccttaccaccaagatcaagttctcagatgtcctgcacaccatcaaggagcatgcctttgtggcctcagagtacccagtcatcctgtccattgaggaccactgcagcattgcccagcagagaaacatggcccaatacttcaagaaggtgctgggggacacactcctcaccaagcccgtggagatctctgccgacgggctcccctcacccaaccagcttaagaggaagatcctcatcaagcacaagaagctggctgagggcagtgcctacgaggaggtgcctacatccatgatgtactctgagaacgacatcagcaactctatcaagaatggcatcctctacctggaggaccctgtgaaccacgaatggtatccccactactttgttctgaccagcagcaagatctactactctgaggagaccagcagtgaccagggcaacgaggatgaggaggagcccaaggaggtcagcagcagcacagagctgcactccaatgagaagtggttccatgggaagctaggggcagggcgtgacgggcgtcacatcgctgagcgcctgcttactgagtactgcatcgagaccggagcccctgacggctccttcctcgtgcgagagagtgagaccttcgtgggcgactacacgctctctttctggcggaacgggaaagtccagcactgccgtatccactcccggcaagatgctgggacccccaagttcttcttgacagacaacctcgtctttgactccctctatgacctcatcacgcactaccagcaggtgcccctgcgctgtaatgagtttgagatgcgactttcagagcctgtcccacagaccaacgcccacgagagcaaagagtggtaccacgcgagcctgaccagagcacaggctgagcacatgctaatgcgcgtccctcgtgatggggccttcctggtgcggaagcggaatgaacccaactcatatgccatctctttccgggctgagggcaagatcaagcattgccgtgtccagcaagagggccagacagtgatgctagggaactcggagttcgacagccttgttgacctcatcagctactatgagaaacacccgctataccgcaagatgaagctgcgctatcccatcaacgaggaggcactggagaagattggcacagctgagcctgactacggggccctgtatgagggacgcaaccctggcttctatgtagaggcaaaccctatgccaactttcaagtgtgcagtcaaagccctctttgactacaaggcccagagggaggacgagctgaccttcaccaagagcgccatcatccagaatgtggagaagcaagagggaggctggtggcgaggggactacggagggaagaagcagctgtggttcccatcaaactacgtggaagagatggtcaaccccgtggccctggagccggagagggagcacttggacgagaacagccccctaggggacttgctgcggggggtcttggatgtgccggcttgtcagattgccatccgtcctgagggcaagaacaaccggctcttcgtcttctccatcagcatggcgtcggtggcccactggtccctggatgttgctgccgactcacaggaggagctgcaggactgggtgaaaaagatccgtgaagtggcccagacagcagacgccaggctcactgaagggaagataatggaacggaggaagaagattgccctggagctctctgaacttgtcgtctactgccggcctgttccctttgatgaagagaagattggcacagaacgtgcttgctaccgggacatgtcatccttcccggaaaccaaggctgagaaatacgtgaacaaggccaaaggcaagaagttccttcagtacaatcgactgcagctctcccgcatctaccccaagggccagcgactggattcctccaactacgatcctttgcccatgtggatctgtggcagtcagcttgtggccctcaacttccagacccctgacaagcctatgcagatgaaccaggccctcttcatgacgggcaggcactgtggctacgtgctgcagccaagcaccatgcgggatgaggccttcgacccctttgacaagagcagcctccgcgggctggagccatgtgccatctctattgaggtgctgggggcccgacatctgccaaagaatggccgaggcattgtgtgtccttttgtggagattgaggtggctggagctgagtatgacagcaccaagcagaagacagagtttgtggtggacaatggactcaaccctgtatggccagccaagcccttccacttccagatcagtaaccctgaatttgcctttctgcgcttcgtggtgtatgaggaacacatgtttagtgaccagaatttcctggctcaggctactttcccagtaaaaggcctgaagacaggatacagagcagtgcctttgaagaacaactacagtgaggacctggagttggcctccctgctgatcaagattgacattttccctgccaagcaggagaatggtgacctcagtcccttcagtggtacgtccctgcgggagcggggctcagatgcctcaggccagctgtttcatggccgagcccgggaaggctcctttgaatcccgctaccagcagccgtttgaggacttccgcatctcccaggagcatctcgcagaccattttgacagtcgagaacgaagggccccaagaaggactcgggtcaatggagacaaccgcctc

SEQ ID NO: 140 - protein, 1291 aa, Synthetic construct
MAGAASPCANGCGPGAPSDAEVLHLCRSLEVGTVMTLFYSKKSQRPERKTFQVKLETRQITWSRGADKIEGAIDIREIKEIRPGKTSRDFDRYQEDPAFRPDQSHCFVILYGMEFRLKTLSLQATSEDEVNMWIKGLTWLMEDTLQAPTPLQIERWLRKQFYSVDRNREDRISAKDLKNMLSQVNYRVPNMRFLRERLTDLEQRSGDITYGQFAQLYRSLMYSAQKTMDLPFLEASTLRAGERPELCRVSLPEFQQFLLDYQGELWAVDRLQVQEFMLSFLRDPLREIEEPYFFLDEFVTFLFSKENSVWNSQLDAVCPDTMNNPLSHYWISSSHNTYLTGDQFSSESSLEAYARCLRMGCRCIELDCWDGPDGMPVIYHGHTLTTKIKFSDVLHTIKEHAFVASEYPVILSIEDHCSIAQQRNMAQYFKKVLGDTLLTKPVEISADGLPSPNQLKRKILIKHKKLAEGSAYEEVPTSMMYSENDISNSIKNGILYLEDPVNHEWYPHYFVLTSSKIYYSEETSSDQGNEDEEEPKEVSSSTELHSNEKWFHGKLGAGRDGRHIAERLLTEYCIETGAPDGSFLVRESETFVGDYTLSFWRNGKVQHCRIHSRQDAGTPKFFLTDNLVFDSLYDLITHYQQVPLRCNEFEMRLSEPVPQTNAHESKEWYHASLTRAQAEHMLMRVPRDGAFLVRKRNEPNSYAISFRAEGKIKHCRVQQEGQTVMLGNSEFDSLVDLISYYEKHPLYRKMKLRYPINEEALEKIGTAEPDYGALYEGRNPGFYVEANPMPTFKCAVKALFDYKAQREDELTFTKSAIIQNVEKQEGGWWRGDYGGKKQLWFPSNYVEEMVNPVALEPEREHLDENSPLGDLLRGVLDVPACQIAIRPEGKNNRLFVFSISMASVAHWSLDVAADSQEELQDWVKKIREVAQTADARLTEGKIMERRKKIALELSELVVYCRPVPFDEEKIGTERACYRDMSSFPETKAEKYVNKAKGKKFLQYNRLQLSRIYPKGQRLDSSNYDPLPMWICGSQLVALNFQTPDKPMQMNQALFMTGRHCGYVLQPSTMRDEAFDPFDKSSLRGLEPCAISIEVLGARHLPKNGRGIVCPFVEIEVAGAEYDSTKQKTEFVVDNGLNPVWPAKPFHFQISNPEFAFLRFVVYEEHMFSDQNFLAQATFPVKGLKTGYRAVPLKNNYSEDLELASLLIKIDIFPAKQENGDLSPFSGTSLRERGSDASGQLFHGRAREGSFESRYQQPFEDFRISQEHLADHFDSRERRAPRRTRVNGDNRL

SEQ ID NO: 141 - DNA, 3873 bp, Homo sapiens
  source 1..3873: mol_type = genomic DNA
atggcgggcgccgcgtccccttgcgccaacggctgcgggcccggcgcgccctcggacgccgaggtgctgcacctctgccgcagcctcgaggtgggcaccgtcatgactttgttctactccaagaagtcgcagcgacccgagcggaagaccttccaggtcaagctggagacgcgccagatcacgtggagccggggcgccgacaagatcgagggggccattgacattcgtgaaattaaggagatccgcccagggaagacctcacgggactttgatcgctatcaagaggacccagctttccggccggaccagtcacattgctttgtcattctctatggaatggaatttcgcctgaaaacgctgagcctgcaagccacatctgaggatgaagtgaacatgtggatcaagggcttaacttggctgatggaggatacattgcaggcacccacacccctgcagattgagaggtggctccggaagcagttttactcagtggatcggaatcgtgaggatcgtatatcagccaaggacctgaagaacatgctgtcccaggtcaactaccgggtccccaacatgcgcttcctccgagagcggctgacggacctggagcagcgcagcggggacatcacctacgggcagtttgctcagctgtaccgcagcctcatgtacagcgcccagaagacgatggacctccccttcttggaagccagtactctgagggctggggagcggccggagctttgccgagtgtcccttcctgagttccagcagttccttcttgactaccagggggagctgtgggctgttgatcgcctccaggtgcaggagttcatgctcagcttcctccgagaccccttacgagagatcgaggagccatacttcttcctggatgagtttgtcaccttcctgttctccaaagagaacagtgtgtggaactcgcagctggatgcagtatgcccggacaccatgaacaaccctctttcccactactggatctcctcctcgcacaacacgtacctgaccggggaccagttctccagtgagtcctccttggaagcctatgctcgctgcctgcggatgggctgtcgctgcattgagttggactgctgggacggcccggatgggatgccagttatttaccatgggcacacccttaccaccaagatcaagttctcagatgtcctgcacaccatcaaggagcatgcctttgtggcctcagagtacccagtcatcctgtccattgaggaccactgcagcattgcccagcagagaaacatggcccaatacttcaagaaggtgctgggggacacactcctcaccaagcccgtggagatctctgccgacgggctcccctcacccaaccagcttaagaggaagatcctcatcaagcacaagaagctggctgagggcagtgcctacgaggaggtgcctacatccatgatgtactctgagaacgacatcagcaactctatcaagaatggcatcctctacctggaggaccctgtgaaccacgaatggtatccccactactttgttctgaccagcagcaagatctactactctgaggagaccagcagtgaccagggcaacgaggatgaggaggagcccaaggaggtcagcagcagcacagagctgcactccaatgagaagtggttccatgggaagctaggggcagggcgtgacgggcgtcacatcgctgagcgcctgcttactgagtactgcatcgagaccggagcccctgacggctccttcctcgtgcgagagagtgagaccttcgtgggcgactacacgctctctttctggcggaacgggaaagtccagcactgccgtatccactcccggcaagatgctgggacccccaagttcttcttgacagacaacctcgtctttgactccctctatgacctcatcacgcactaccagcaggtgcccctgcgctgtaatgagtttgagatgcgactttcagagcctgtcccacagaccaacgcccacgagagcaaagagtggtaccacgcgagcctgaccagagcacaggctgagcacatgctaatgcgcgtccctcgtgatggggccttcctggtgcggaagcggaatgaacccaactcatatgccatctctttccgggctgagggcaagatcaagcattgccgtgtccagcaagagggccagacagtgatgctagggaactcggagttcgacagccttgttgacctcatcagctactatgagaaacacccgctataccgcaagatgaagctgcgctatcccatcaacgaggaggcactggagaagattggcacagctgagcctgactacggggccctgtatgagggacgcaaccctggcttctatgtagaggcaaaccctatgccaactttcaagtgtgcagtcaaagccctctttgactacaaggcccagagggaggacgagctgaccttcaccaagagcgccatcatccagaatgtggagaagcaagagggaggctggtggcgaggggactacggagggaagaagcagctgtggttcccatcaaactacgtggaagagatggtcaaccccgtggccctggagccggagagggagcacttggacgagaacagccccctaggggacttgctgcggggggtcttggatgtgccggcttgtcagattgccatccgtcctgagggcaagaacaaccggctcttcgtcttctccatcagcatggcgtcggtggcccactggtccctggatgttgctgccgactcacaggaggagctgcaggactgggtgaaaaagatccgtgaagtggcccagacagcagacgccaggctcactgaagggaagataatggaacggaggaagaagattgccctggagctctctgaacttgtcgtctactgccggcctgttccctttgatgaagagaagattggcacagaacgtgcttgctaccgggacatgtcatccttcccggaaaccaaggctgagaaatacgtgaacaaggccaaaggcaagaagttccttcagtacaatcgactgcagctctcccgcatctaccccaagggccagcgactggattcctccaactacgatcctttgcccatgtggatctgtggcagtcagcttgtggccctcaacttccagacccctgacaagcctatgcagatgaaccaggccctcttcatgacgggcaggcactgtggctacgtgctgcagccaagcaccatgcgggatgaggccttcgacccctttgacaagagcagcctccgcgggctggagccatgtgccatctctattgaggtgctgggggcccgacatctgccaaagaatggccgaggcattgtgtgtccttttgtggagattgaggtggctggagctgagtatgacagcaccaagcagaagacagagtttgtggtggacaatggactcaaccctgtatggccagccaagcccttccacttccagatcagtaaccctgaatttgcctttctgcgcttcgtggtgtatgaggaagacatgtttagtgaccagaatttcctggctcaggctactttcccagtaaaaggcctgaagacaggatacagagcagtgcctttgaagaacaactacagtgaggacctggagttggcctccctgctgatcaagattgacattttccctgccaagcaggagaatggtgacctcagtcccttcagtggtacgtccctgcgggagcggggctcagatgcctcaggccagctgtttcatggccgagcccgggaaggctcctttgaatcccgctaccagcagccgtttgaggacttccgcatctcccaggagcatctcgcagaccattttgacagtcgagaacgaagggccccaagaaggactcgggtcaatggagacaaccgcctc

SEQ ID NO: 142 - protein, 1291 aa, Homo sapiens
MAGAASPCANGCGPGAPSDAEVLHLCRSLEVGTVMTLFYSKKSQRPERKTFQVKLETRQITWSRGADKIEGAIDIREIKEIRPGKTSRDFDRYQEDPAFRPDQSHCFVILYGMEFRLKTLSLQATSEDEVNMWIKGLTWLMEDTLQAPTPLQIERWLRKQFYSVDRNREDRISAKDLKNMLSQVNYRVPNMRFLRERLTDLEQRSGDITYGQFAQLYRSLMYSAQKTMDLPFLEASTLRAGERPELCRVSLPEFQQFLLDYQGELWAVDRLQVQEFMLSFLRDPLREIEEPYFFLDEFVTFLFSKENSVWNSQLDAVCPDTMNNPLSHYWISSSHNTYLTGDQFSSESSLEAYARCLRMGCRCIELDCWDGPDGMPVIYHGHTLTTKIKFSDVLHTIKEHAFVASEYPVILSIEDHCSIAQQRNMAQYFKKVLGDTLLTKPVEISADGLPSPNQLKRKILIKHKKLAEGSAYEEVPTSMMYSENDISNSIKNGILYLEDPVNHEWYPHYFVLTSSKIYYSEETSSDQGNEDEEEPKEVSSSTELHSNEKWFHGKLGAGRDGRHIAERLLTEYCIETGAPDGSFLVRESETFVGDYTLSFWRNGKVQHCRIHSRQDAGTPKFFLTDNLVFDSLYDLITHYQQVPLRCNEFEMRLSEPVPQTNAHESKEWYHASLTRAQAEHMLMRVPRDGAFLVRKRNEPNSYAISFRAEGKIKHCRVQQEGQTVMLGNSEFDSLVDLISYYEKHPLYRKMKLRYPINEEALEKIGTAEPDYGALYEGRNPGFYVEANPMPTFKCAVKALFDYKAQREDELTFTKSAIIQNVEKQEGGWWRGDYGGKKQLWFPSNYVEEMVNPVALEPEREHLDENSPLGDLLRGVLDVPACQIAIRPEGKNNRLFVFSISMASVAHWSLDVAADSQEELQDWVKKIREVAQTADARLTEGKIMERRKKIALELSELVVYCRPVPFDEEKIGTERACYRDMSSFPETKAEKYVNKAKGKKFLQYNRLQLSRIYPKGQRLDSSNYDPLPMWICGSQLVALNFQTPDKPMQMNQALFMTGRHCGYVLQPSTMRDEAFDPFDKSSLRGLEPCAISIEVLGARHLPKNGRGIVCPFVEIEVAGAEYDSTKQKTEFVVDNGLNPVWPAKPFHFQISNPEFAFLRFVVYEEDMFSDQNFLAQATFPVKGLKTGYRAVPLKNNYSEDLELASLLIKIDIFPAKQENGDLSPFSGTSLRERGSDASGQLFHGRAREGSFESRYQQPFEDFRISQEHLADHFDSRERRAPRRTRVNGDNRL

SEQ ID NO: 143 - DNA, 3873 bp, Synthetic construct
  source 1..3873: mol_type = other DNA
atggcgggcgccgcgtccccttgcgccaacggctgcgggcccggcgcgccctcggacgccgaggtgctgcacctctgccgcagcctcgaggtgggcaccgtcatgactttgttctactccaagaagtcgcagcgacccgagtggaagaccttccaggtcaagctggagacgcgccagatcacgtggagccggggcgccgacaagatcgagggggccattgacattcgtgaaattaaggagatccgcccagggaagacctcacgggactttgatcgctatcaagaggacccagctttccggccggaccagtcacattgctttgtcattctctatggaatggaatttcgcctgaaaacgctgagcctgcaagccacatctgaggatgaagtgaacatgtggatcaagggcttaacttggctgatggaggatacattgcaggcacccacacccctgcagattgagaggtggctccggaagcagttttactcagtggatcggaatcgtgaggatcgtatatcagccaaggacctgaagaacatgctgtcccaggtcaactaccgggtccccaacatgcgcttcctccgagagcggctgacggacctggagcagcgcagcggggacatcacctacgggcagtttgctcagctgtaccgcagcctcatgtacagcgcccagaagacgatggacctccccttcttggaagccagtactctgagggctggggagcggccggagctttgccgagtgtcccttcctgagttccagcagttccttcttgactaccagggggagctgtgggctgttgatcgcctccaggtgcaggagttcatgctcagcttcctccgagaccccttacgagagatcgaggagccatacttcttcctggatgagtttgtcaccttcctgttctccaaagagaacagtgtgtggaactcgcagctggatgcagtatgcccggacaccatgaacaaccctctttcccactactggatctcctcctcgcacaacacgtacctgaccggggaccagttctccagtgagtcctccttggaagcctatgctcgctgcctgcggatgggctgtcgctgcattgagttggactgctgggacggcccggatgggatgccagttatttaccatgggcacacccttaccaccaagatcaagttctcagatgtcctgcacaccatcaaggagcatgcctttgtggcctcagagtacccagtcatcctgtccattgaggaccactgcagcattgcccagcagagaaacatggcccaatacttcaagaaggtgctgggggacacactcctcaccaagcccgtggagatctctgccgacgggctcccctcacccaaccagcttaagaggaagatcctcatcaagcacaagaagctggctgagggcagtgcctacgaggaggtgcctacatccatgatgtactctgagaacgacatcagcaactctatcaagaatggcatcctctacctggaggaccctgtgaaccacgaatggtatccccactactttgttctgaccagcagcaagatctactactctgaggagaccagcagtgaccagggcaacgaggatgaggaggagcccaaggaggtcagcagcagcacagagctgcactccaatgagaagtggttccatgggaagctaggggcagggcgtgacgggcgtcacatcgctgagcgcctgcttactgagtactgcatcgagaccggagcccctgacggctccttcctcgtgcgagagagtgagaccttcgtgggcgactacacgctctctttctggcggaacgggaaagtccagcactgccgtatccactcccggcaagatgctgggacccccaagttcttcttgacagacaacctcgtctttgactccctctatgacctcatcacgcactaccagcaggtgcccctgcgctgtaatgagtttgagatgcgactttcagagcctgtcccacagaccaacgcccacgagagcaaagagtggtaccacgcgagcctgaccagagcacaggctgagcacatgctaatgcgcgtccctcgtgatggggccttcctggtgcggaagcggaatgaacccaactcatatgccatctctttccgggctgagggcaagatcaagcattgccgtgtccagcaagagggccagacagtgatgctagggaactcggagttcgacagccttgttgacctcatcagctactatgagaaacacccgctataccgcaagatgaagctgcgctatcccatcaacgaggaggcactggagaagattggcacagctgagcctgactacggggccctgtatgagggacgcaaccctggcttctatgtagaggcaaaccctatgccaactttcaagtgtgcagtcaaagccctctttgactacaaggcccagagggaggacgagctgaccttcaccaagagcgccatcatccagaatgtggagaagcaagagggaggctggtggcgaggggactacggagggaagaagcagctgtggttcccatcaaactacgtggaagagatggtcaaccccgtggccctggagccggagagggagcacttggacgagaacagccccctaggggacttgctgcggggggtcttggatgtgccggcttgtcagattgccatccgtcctgagggcaagaacaaccggctcttcgtcttctccatcagcatggcgtcggtggcccactggtccctggatgttgctgccgactcacaggaggagctgcaggactgggtgaaaaagatccgtgaagtggcccagacagcagacgccaggctcactgaagggaagataatggaacggaggaagaagattgccctggagctctctgaacttgtcgtctactgccggcctgttccctttgatgaagagaagattggcacagaacgtgcttgctaccgggacatgtcatccttcccggaaaccaaggctgagaaatacgtgaacaaggccaaaggcaagaagttccttcagtacaatcgactgcagctctcccgcatctaccccaagggccagcgactggattcctccaactacgatcctttgcccatgtggatctgtggcagtcagcttgtggccctcaacttccagacccctgacaagcctatgcagatgaaccaggccctcttcatgacgggcaggcactgtggctacgtgctgcagccaagcaccatgcgggatgaggccttcgacccctttgacaagagcagcctccgcgggctggagccatgtgccatctctattgaggtgctgggggcccgacatctgccaaagaatggccgaggcattgtgtgtccttttgtggagattgaggtggctggagctgagtatgacagcaccaagcagaagacagagtttgtggtggacaatggactcaaccctgtatggccagccaagcccttccacttccagatcagtaaccctgaatttgcctttctgcgcttcgtggtgtatgaggaagacatgtttagtgaccagaatttcctggctcaggctactttcccagtaaaaggcctgaagacaggatacagagcagtgcctttgaagaacaactacagtgaggacctggagttggcctccctgctgatcaagattgacattttccctgccaagcaggagaatggtgacctcagtcccttcagtggtacgtccctgcgggagcggggctcagatgcctcaggccagctgtttcatggccgagcccgggaaggctcctttgaatcccgctaccagcagccgtttgaggacttccgcatctcccaggagcatctcgcagaccattttgacagtcgagaacgaagggccccaagaaggactcgggtcaatggagacaaccgcctc

SEQ ID NO: 144 - protein, 1291 aa, Synthetic construct
MAGAASPCANGCGPGAPSDAEVLHLCRSLEVGTVMTLFYSKKSQRPEWKTFQVKLETRQITWSRGADKIEGAIDIREIKEIRPGKTSRDFDRYQEDPAFRPDQSHCFVILYGMEFRLKTLSLQATSEDEVNMWIKGLTWLMEDTLQAPTPLQIERWLRKQFYSVDRNREDRISAKDLKNMLSQVNYRVPNMRFLRERLTDLEQRSGDITYGQFAQLYRSLMYSAQKTMDLPFLEASTLRAGERPELCRVSLPEFQQFLLDYQGELWAVDRLQVQEFMLSFLRDPLREIEEPYFFLDEFVTFLFSKENSVWNSQLDAVCPDTMNNPLSHYWISSSHNTYLTGDQFSSESSLEAYARCLRMGCRCIELDCWDGPDGMPVIYHGHTLTTKIKFSDVLHTIKEHAFVASEYPVILSIEDHCSIAQQRNMAQYFKKVLGDTLLTKPVEISADGLPSPNQLKRKILIKHKKLAEGSAYEEVPTSMMYSENDISNSIKNGILYLEDPVNHEWYPHYFVLTSSKIYYSEETSSDQGNEDEEEPKEVSSSTELHSNEKWFHGKLGAGRDGRHIAERLLTEYCIETGAPDGSFLVRESETFVGDYTLSFWRNGKVQHCRIHSRQDAGTPKFFLTDNLVFDSLYDLITHYQQVPLRCNEFEMRLSEPVPQTNAHESKEWYHASLTRAQAEHMLMRVPRDGAFLVRKRNEPNSYAISFRAEGKIKHCRVQQEGQTVMLGNSEFDSLVDLISYYEKHPLYRKMKLRYPINEEALEKIGTAEPDYGALYEGRNPGFYVEANPMPTFKCAVKALFDYKAQREDELTFTKSAIIQNVEKQEGGWWRGDYGGKKQLWFPSNYVEEMVNPVALEPEREHLDENSPLGDLLRGVLDVPACQIAIRPEGKNNRLFVFSISMASVAHWSLDVAADSQEELQDWVKKIREVAQTADARLTEGKIMERRKKIALELSELVVYCRPVPFDEEKIGTERACYRDMSSFPETKAEKYVNKAKGKKFLQYNRLQLSRIYPKGQRLDSSNYDPLPMWICGSQLVALNFQTPDKPMQMNQALFMTGRHCGYVLQPSTMRDEAFDPFDKSSLRGLEPCAISIEVLGARHLPKNGRGIVCPFVEIEVAGAEYDSTKQKTEFVVDNGLNPVWPAKPFHFQISNPEFAFLRFVVYEEDMFSDQNFLAQATFPVKGLKTGYRAVPLKNNYSEDLELASLLIKIDIFPAKQENGDLSPFSGTSLRERGSDASGQLFHGRAREGSFESRYQQPFEDFRISQEHLADHFDSRERRAPRRTRVNGDNRL

SEQ ID NO: 145 - DNA, 2019 bp, Synthetic construct
  source 1..2019: mol_type = other DNA
atggctgacccggctgcggggccgccgccgagcgagggcgaggagagcaccgtgcgcttcgcccgcaaaggcgccctccggcagaagaacgtgcatgaggtcaagaaccacaaattcaccgcccgcttcttcaagcagcccaccttctgcagccactgcaccgacttcatctggggcttcgggaagcagggattccagtgccaagtttgctgctttgtggtgcacaagcggtgccatgaatttgtcacattctcctgccctggcgctgacaagggtccagcctccgatgacccccgcagcaaacacaagtttaagatccacacgtactccagccccacgttttgtgaccactgtgggtcactgctgtatggactcatccaccaggggatgaaatgtgacacctgcatgatgaatgtgcacaagcgctgcgtgatgaatgttcccagcctgtgtggcacggaccacacggagcgccgcggccgcatctacatccaggcccacatcgacagggacgtcctcattgtcctcgtaagagatgctaaaaaccttgtacctatggaccccaatggcctgtcagatccctacgtaaaactgaaactgattcccgatcccaaaagtgagagcaaacagaagaccaaaaccatcaaatgctccctcaaccctgagtggaatgagacatttagatttcagctgaaagaatcggacaaagacagaagactgtcagtagagatttgggattgggatttgaccagcaggaatgacttcatgggatctttgtcctttgggatttctgaacttcagaaagccagtgttgatggctggtttaagttactgagccaggaggaaggcgagtacttcaatgtgcctgtgccaccagaaggaagtgaggccaatgaagaactgcggcagaaatttgagagggccaagatcagtcagggaaccaaggtcccggaagaaaagacgaccaacactgtctccaaatttgacaacaatggcaacagagaccggatgaaactgaccgattttaacttcctaatggtgctggggaaaggcagctttggcaaggtcatgctttcagaacgaaaaggcacagatgagctctatgctgtgaagatcctgaagaaggacgttgtgatccaagatgatgacgtggagtgcactatggtggagaagcgggtgttggccctgcctgggaagccgcccttcctgacccagctccactcctgcttccagaccatggaccgcctgtactttgtgatggagtacgtgaatgggggcgacctcatgtatcacatccagcaagtcggccggttcaaggagccccatgctgtattttacgctgcagaaattgccatcggtctgttcttcttacagagtaagggcatcatttaccgtgacctaaaacttgacaacgtgatgctcgattctgagggacacatcaagattgccgattttggcatgtgtaaggaaaacatctgggatggggtgacaaccaagacattctgtggcactccagactacatcgcccccgagataattgcttatcagccctatgggaagtccgtggattggtgggcatttggagtcctgctgtatgaaatgttggctgggcaggcaccctttgaaggggaggatgaagatgaactcttccaatccatcatggaacacaacgtagcctatcccaagtctatgtccaaggaagctgtggccatctgcaaagggctgatgaccaaacacccaggcaaacgtctgggttgtggacctgaaggcgaacgtgatatcaaagagcatgcatttttccggtatattgattgggagaaacttgaacgcaaagagatccagcccccttataagccaaaagcttgtgggcgaaatgctgaaaacttctaccgatttttcacccgccatccaccagtcctaacacctcccgaccaggaagtcatcaggaatattgaccaatcagaattcgaaggattttcctttgttaactctgaatttttaaaacccgaagtcaagagc

SEQ ID NO: 146 - protein, 673 aa, Synthetic construct
MADPAAGPPPSEGEESTVRFARKGALRQKNVHEVKNHKFTARFFKQPTFCSHCTDFIWGFGKQGFQCQVCCFVVHKRCHEFVTFSCPGADKGPASDDPRSKHKFKIHTYSSPTFCDHCGSLLYGLIHQGMKCDTCMMNVHKRCVMNVPSLCGTDHTERRGRIYIQAHIDRDVLIVLVRDAKNLVPMDPNGLSDPYVKLKLIPDPKSESKQKTKTIKCSLNPEWNETFRFQLKESDKDRRLSVEIWDWDLTSRNDFMGSLSFGISELQKASVDGWFKLLSQEEGEYFNVPVPPEGSEANEELRQKFERAKISQGTKVPEEKTTNTVSKFDNNGNRDRMKLTDFNFLMVLGKGSFGKVMLSERKGTDELYAVKILKKDVVIQDDDVECTMVEKRVLALPGKPPFLTQLHSCFQTMDRLYFVMEYVNGGDLMYHIQQVGRFKEPHAVFYAAEIAIGLFFLQSKGIIYRDLKLDNVMLDSEGHIKIADFGMCKENIWDGVTTKTFCGTPDYIAPEIIAYQPYGKSVDWWAFGVLLYEMLAGQAPFEGEDEDELFQSIMEHNVAYPKSMSKEAVAICKGLMTKHPGKRLGCGPEGERDIKEHAFFRYIDWEKLERKEIQPPYKPKACGRNAENFYRFFTRHPPVLTPPDQEVIRNIDQSEFEGFSFVNSEFLKPEVKS

SEQ ID NO: 147 - DNA, 2019 bp, Homo sapiens
  source 1..2019: mol_type = genomic DNA
atggctgacccggctgcggggccgccgccgagcgagggcgaggagagcaccgtgcgcttcgcccgcaaaggcgccctccggcagaagaacgtgcatgaggtcaagaaccacaaattcaccgcccgcttcttcaagcagcccaccttctgcagccactgcaccgacttcatctggggcttcgggaagcagggattccagtgccaagtttgctgctttgtggtgcacaagcggtgccatgaatttgtcacattctcctgccctggcgctgacaagggtccagcctccgatgacccccgcagcaaacacaagtttaagatccacacgtactccagccccacgttttgtgaccactgtgggtcactgctgtatggactcatccaccaggggatgaaatgtgacacctgcatgatgaatgtgcacaagcgctgcgtgatgaatgttcccagcctgtgtggcacggaccacacggagcgccgcggccgcatctacatccaggcccacatcgacagggacgtcctcattgtcctcgtaagagatgctaaaaaccttgtacctatggaccccaatggcctgtcagatccctacgtaaaactgaaactgattcccgatcccaaaagtgagagcaaacagaagaccaaaaccatcaaatgctccctcaaccctgagtggaatgagacatttagatttcagctgaaagaatcggacaaagacagaagactgtcagtagagatttgggattgggatttgaccagcaggaatgacttcatgggatctttgtcctttgggatttctgaacttcagaaagccagtgttgatggctggtttaagttactgagccaggaggaaggcgagtacttcaatgtgcctgtgccaccagaaggaagtgaggccaatgaagaactgcggcagaaatttgagagggccaagatcagtcagggaaccaaggtcccggaagaaaagacgaccaacactgtctccaaatttgacaacaatggcaacagagaccggatgaaactgaccgattttaacttcctaatggtgctggggaaaggcagctttggcaaggtcatgctttcagaacgaaaaggcacagatgagctctatgctgtgaagatcctgaagaaggacgttgtgatccaagatgatgacgtggagtgcactatggtggagaagcgggtgttggccctgcctgggaagccgcccttcctgacccagctccactcctgcttccagaccatggaccgcctgtactttgtgatggagtacgtgaatgggggcgacctcatgtatcacatccagcaagtcggccggttcaaggagccccatgctgtattttacgctgcagaaattgccatcggtctgttcttcttacagagtaagggcatcatttaccgtgacctaaaacttgacaacgtgatgctcgattctgagggacacatcaagattgccgattttggcatgtgtaaggaaaacatctgggatggggtgacaaccaagacattctgtggcactccagactacatcgcccccgagataattgcttatcagccctatgggaagtccgtggattggtgggcatttggagtcctgctgtatgaaatgttggctgggcaggcaccctttgaaggggaggatgaagatgaactcttccaatccatcatggaacacaacgtagcctatcccaagtctatgtccaaggaagctgtggccatctgcaaagggctgatgaccaaacacccaggcaaacgtctgggttgtggacctgaaggcgaacgtgatatcaaagagcatgcatttttccggtatattgattgggagaaacttgaacgcaaagagatccagcccccttataagccaaaagcttgtgggcgaaatgctgaaaacttcgaccgatttttcacccgccatccaccagtcctaacacctcccgaccaggaagtcatcaggaatattgaccaatcagaattcgaaggattttcctttgttaactctgaatttttaaaacccgaagtcaagagc

SEQ ID NO: 148 - protein, 673 aa, Homo sapiens
MADPAAGPPPSEGEESTVRFARKGALRQKNVHEVKNHKFTARFFKQPTFCSHCTDFIWGFGKQGFQCQVCCFVVHKRCHEFVTFSCPGADKGPASDDPRSKHKFKIHTYSSPTFCDHCGSLLYGLIHQGMKCDTCMMNVHKRCVMNVPSLCGTDHTERRGRIYIQAHIDRDVLIVLVRDAKNLVPMDPNGLSDPYVKLKLIPDPKSESKQKTKTIKCSLNPEWNETFRFQLKESDKDRRLSVEIWDWDLTSRNDFMGSLSFGISELQKASVDGWFKLLSQEEGEYFNVPVPPEGSEANEELRQKFERAKISQGTKVPEEKTTNTVSKFDNNGNRDRMKLTDFNFLMVLGKGSFGKVMLSERKGTDELYAVKILKKDVVIQDDDVECTMVEKRVLALPGKPPFLTQLHSCFQTMDRLYFVMEYVNGGDLMYHIQQVGRFKEPHAVFYAAEIAIGLFFLQSKGIIYRDLKLDNVMLDSEGHIKIADFGMCKENIWDGVTTKTFCGTPDYIAPEIIAYQPYGKSVDWWAFGVLLYEMLAGQAPFEGEDEDELFQSIMEHNVAYPKSMSKEAVAICKGLMTKHPGKRLGCGPEGERDIKEHAFFRYIDWEKLERKEIQPPYKPKACGRNAENFDRFFTRHPPVLTPPDQEVIRNIDQSEFEGFSFVNSEFLKPEVKS

SEQ ID NO: 149 - DNA, 2019 bp, Synthetic construct
  source 1..2019: mol_type = other DNA
atggctgacccggctgcggggccgccgccgagcgagggcgaggagagcaccgtgcgcttcgcccgcaaaggcgccctccggcagaagaacgtgcatgaggtcaagaaccacaaattcaccgcccgcttcttcaagcagcccaccttctgcagccactgcaccgacttcatctggggcttcgggaagcagggattccagtgccaagtttgctgctttgtggtgcacaagcggtgccatgaatttgtcacattctcctgccctggcgctgacaagggtccagcctccgatgacccccgcagcaaacacaagtttaagatccacacgtactccagccccacgttttgtgaccactgtgggtcactgctgtatggactcatccaccaggggatgaaatgtgacacctgcatgatgaatgtgcacaagcgctgcgtgatgaatgttcccagcctgtgtggcacggaccacacggagcgccgcggccgcatctacatccaggcccacatcgacagggacgtcctcattgtcctcgtaagagatgctaaaaaccttgtacctatggaccccaatggcctgtcagatccctacgtaaaactgaaactgattcccgatcccaaaagtgagagcaaacagaagaccaaaaccatcaaatgctccctcaaccctgagtggaatgagacatttagatttcagctgaaagaatcggacaaagacagaagactgtcagtagagatttgggattgggatttgaccagcaggaatgacttcatgggatctttgtcctttgggatttctgaacttcagaaagccagtgttgatggctggtttaagttactgagccaggaggaaggcgagtacttcaatgtgcctgtgccaccagaaggaagtgaggccaatgaagaactgcggcagaaatttgagagggccaagatcagtcagggaaccaaggtcccggaagaaaagacgaccaacactgtctccaaatttgacaacaatggcaacagagaccggatgaaactgaccgattttaacttcctaatggtgctggggaaaggcagctttggcaaggtcatgctttcagaacgaaaaggcacagatgagctctatgctgtgaagatcctgaagaaggacgttgtgatccaagatgatgacgtggagtgcactatggtggagaagcgggtgttggccctgcctgggaagccgcccttcctgacccagctccactcctgcttccagaccatggaccgcctgtactttgtgatggagtacgtgaatgggggcaacctcatgtatcacatccagcaagtcggccggttcaaggagccccatgctgtattttacgctgcagaaattgccatcggtctgttcttcttacagagtaagggcatcatttaccgtgacctaaaacttgacaacgtgatgctcgattctgagggacacatcaagattgccgattttggcatgtgtaaggaaaacatctgggatggggtgacaaccaagacattctgtggcactccagactacatcgcccccgagataattgcttatcagccctatgggaagtccgtggattggtgggcatttggagtcctgctgtatgaaatgttggctgggcaggcaccctttgaaggggaggatgaagatgaactcttccaatccatcatggaacacaacgtagcctatcccaagtctatgtccaaggaagctgtggccatctgcaaagggctgatgaccaaacacccaggcaaacgtctgggttgtggacctgaaggcgaacgtgatatcaaagagcatgcatttttccggtatattgattgggagaaacttgaacgcaaagagatccagcccccttataagccaaaagcttgtgggcgaaatgctgaaaacttcgaccgatttttcacccgccatccaccagtcctaacacctcccgaccaggaagtcatcaggaatattgaccaatcagaattcgaaggattttcctttgttaactctgaatttttaaaacccgaagtcaagagc

SEQ ID NO: 150 - protein, 673 aa, Synthetic construct
MADPAAGPPPSEGEESTVRFARKGALRQKNVHEVKNHKFTARFFKQPTFCSHCTDFIWGFGKQGFQCQVCCFVVHKRCHEFVTFSCPGADKGPASDDPRSKHKFKIHTYSSPTFCDHCGSLLYGLIHQGMKCDTCMMNVHKRCVMNVPSLCGTDHTERRGRIYIQAHIDRDVLIVLVRDAKNLVPMDPNGLSDPYVKLKLIPDPKSESKQKTKTIKCSLNPEWNETFRFQLKESDKDRRLSVEIWDWDLTSRNDFMGSLSFGISELQKASVDGWFKLLSQEEGEYFNVPVPPEGSEANEELRQKFERAKISQGTKVPEEKTTNTVSKFDNNGNRDRMKLTDFNFLMVLGKGSFGKVMLSERKGTDELYAVKILKKDVVIQDDDVECTMVEKRVLALPGKPPFLTQLHSCFQTMDRLYFVMEYVNGGNLMYHIQQVGRFKEPHAVFYAAEIAIGLFFLQSKGIIYRDLKLDNVMLDSEGHIKIADFGMCKENIWDGVTTKTFCGTPDYIAPEIIAYQPYGKSVDWWAFGVLLYEMLAGQAPFEGEDEDELFQSIMEHNVAYPKSMSKEAVAICKGLMTKHPGKRLGCGPEGERDIKEHAFFRYIDWEKLERKEIQPPYKPKACGRNAENFDRFFTRHPPVLTPPDQEVIRNIDQSEFEGFSFVNSEFLKPEVKS

SEQ ID NO: 151 - DNA, 1386 bp, Homo sapiens
  source 1..1386: mol_type = genomic DNA
atggccagcaacagcagctcctgcccgacacctgggggcgggcacctcaatgggtacccggtgcctccctacgccttcttcttcccccctatgctgggtggactctccccgccaggcgctctgaccactctccagcaccagcttccagttagtggatatagcacaccatccccagccaccattgagacccagagcagcagttctgaagagatagtgcccagccctccctcgccaccccctctaccccgcatctacaagccttgctttgtctgtcaggacaagtcctcaggctaccactatggggtcagcgcctgtgagggctgcaagggcttcttccgccgcagcatccagaagaacatggtgtacacgtgtcaccgggacaagaactgcatcatcaacaaggtgacccggaaccgctgccagtactgccgactgcagaagtgctttgaagtgggcatgtccaaggagtctgtgagaaacgaccgaaacaagaagaagaaggaggtgcccaagcccgagtgctctgagagctacacgctgacgccggaggtgggggagctcattgagaaggtgcgcaaagcgcaccaggaaaccttccctgccctctgccagctgggcaaatacactacgaacaacagctcagaacaacgtgtctctctggacattgacctctgggacaagttcagtgaactctccaccaagtgcatcattaagactgtggagttcgccaagcagctgcccggcttcaccaccctcaccatcgccgaccagatcaccctcctcaaggctgcctgcctggacatcctgatcctgcggatctgcacgcggtacacgcccgagcaggacaccatgaccttctcggacgggctgaccctgaaccggacccagatgcacaacgctggcttcggccccctcaccgacctggtctttgccttcgccaaccagctgctgcccctggagatggatgatgcggagacggggctgctcagcgccatctgcctcatctgcggagaccgccaggacctggagcagccggaccgggtggacatgctgcaggagccgctgctggaggcgctaaaggtctacgtgcggaagcggaggcccagccgcccccacatgttccccaagatgctaatgaagattactgacctgcgaagcatcagcgccaagggggctgagcgggtgatcacgctgaagatggagatcccgggctccatgccgcctctcatccaggaaatgttggagaactcagagggcctggacactctgagcggacagccggggggtggggggcgggacgggggtggcctggcccccccgccaggcagctgtagccccagcctcagccccagctccaacagaagcagcccggccacccactccccg

SEQ ID NO: 152 - protein, 462 aa, Homo sapiens
MASNSSSCPTPGGGHLNGYPVPPYAFFFPPMLGGLSPPGALTTLQHQLPVSGYSTPSPATIETQSSSSEEIVPSPPSPPPLPRIYKPCFVCQDKSSGYHYGVSACEGCKGFFRRSIQKNMVYTCHRDKNCIINKVTRNRCQYCRLQKCFEVGMSKESVRNDRNKKKKEVPKPECSESYTLTPEVGELIEKVRKAHQETFPALCQLGKYTTNNSSEQRVSLDIDLWDKFSELSTKCIIKTVEFAKQLPGFTTLTIADQITLLKAACLDILILRICTRYTPEQDTMTFSDGLTLNRTQMHNAGFGPLTDLVFAFANQLLPLEMDDAETGLLSAICLICGDRQDLEQPDRVDMLQEPLLEALKVYVRKRRPSRPHMFPKMLMKITDLRSISAKGAERVITLKMEIPGSMPPLIQEMLENSEGLDTLSGQPGGGGRDGGGLAPPPGSCSPSLSPSSNRSSPATHSP

SEQ ID NO: 153 - DNA, 1386 bp, Synthetic construct
  source 1..1386: mol_type = other DNA
atggccagcaacagcagctcctgcccgacacctgggggcgggcacctcaatgggtacccggtgcctccctacgccttcttcttcccccctatgctgggtggactctccccgccaggcgctctgaccactctccagcaccagcttccagttagtggatatagcacaccatccccagccaccattgagacccagagcagcagttctgaagagatagtgcccagccctccctcgccaccccctctaccccgcatctacaagccttgctttgtctgtcaggacaagtcctcaggctaccactatggggtcagcgcctgtgagggctgcaagggcttcttccgccgcagcatccagaagaacatggtgtacacgtgtcaccgggacaagaactgcatcatcaacaaggtgacccggaaccgctgccagtactgccgactgcagaagtgctttgaagtgggcatgtccaaggagtctgtgagaaacgaccgaaacaagaagaagaaggaggtgcccaagcccgagtgctctgagagctacacgctgacgccggaggtgggggagctcattgagaaggtgcgcaaagcgcaccaggaaaccttccctgccctctgccagctgagcaaatacactacgaacaacagctcagaacaacgtgtctctctggacattgacctctgggacaagttcagtgaactctccaccaagtgcatcattaagactgtggagttcgccaagcagctgcccggcttcaccaccctcaccatcgccgaccagatcaccctcctcaaggctgcctgcctggacatcctgatcctgcggatctgcacgcggtacacgcccgagcaggacaccatgaccttctcggacgggctgaccctgaaccggacccagatgcacaacgctggcttcggccccctcaccgacctggtctttgccttcgccaaccagctgctgcccctggagatggatgatgcggagacggggctgctcagcgccatctgcctcatctgcggagaccgccaggacctggagcagccggaccgggtggacatgctgcaggagccgctgctggaggcgctaaaggtctacgtgcggaagcggaggcccagccgcccccacatgttccccaagatgctaatgaagattactgacctgcgaagcatcagcgccaagggggctgagcgggtgatcacgctgaagatggagatcccgggctccatgccgcctctcatccaggaaatgttggagaactcagagggcctggacactctgagcggacagccggggggtggggggcgggacgggggtggcctggcccccccgccaggcagctgtagccccagcctcagccccagctccaacagaagcagcccggccacccactccccg

SEQ ID NO: 154 - protein, 462 aa, Synthetic construct
MASNSSSCPTPGGGHLNGYPVPPYAFFFPPMLGGLSPPGALTTLQHQLPVSGYSTPSPATIETQSSSSEEIVPSPPSPPPLPRIYKPCFVCQDKSSGYHYGVSACEGCKGFFRRSIQKNMVYTCHRDKNCIINKVTRNRCQYCRLQKCFEVGMSKESVRNDRNKKKKEVPKPECSESYTLTPEVGELIEKVRKAHQETFPALCQLSKYTTNNSSEQRVSLDIDLWDKFSELSTKCIIKTVEFAKQLPGFTTLTIADQITLLKAACLDILILRICTRYTPEQDTMTFSDGLTLNRTQMHNAGFGPLTDLVFAFANQLLPLEMDDAETGLLSAICLICGDRQDLEQPDRVDMLQEPLLEALKVYVRKRRPSRPHMFPKMLMKITDLRSISAKGAERVITLKMEIPGSMPPLIQEMLENSEGLDTLSGQPGGGGRDGGGLAPPPGSCSPSLSPSSNRSSPATHSP

SEQ ID NO: 155 - DNA, 2391 bp, Synthetic construct
  source 1..2391: mol_type = other DNA
atgggcaccctgggcaaggcgagagaggctccgcggaaaccttcccatggctgcagagctgcctctaaagcaagactagaggcaaagccagccaacagccccttcccctcccatcccagcttggcccacatcacccagttccgaatgatggtgtctctgggacatttagccaaaggagccagcctggacgatctcattgacagctgcattcaatcttttgatgcagatggaaacctgtgtcgaagtaaccaactgttgcaagtcatgctgaccatgcaccgaattgtcatctcctctgcagaactgctccaaaaagttatcaccctctataaggatgctttggcaaagaattcaccaggactttgcctgaagatctgttattttgtaaggtattggataacagaattctgggtcatgtttaaaatggacgccagcttgacagacactatggaggagtttcaggaactggtgaaagctaagggtgaggagttacattgccgcctgattgacacaactcaaatcaatgcccgtgactggtccaggaaacttactcaaaggataaaatcaaataccagcaagaaacggaaagtctccctgctctttgaccatctggaaccagaagagctatccgagcacctcacctaccttgagttcaagtctttccggaggatatcgttctctgattatcagaattaccttgtaaatagctgtgtgaaggaaaaccccaccatggagcgatctattgctctgtgcaacggcatctcccagtgggtacaactgatcgttctcagccgccccacgccgcagctccgagcagaagtcttcatcaagttcatccaggtggctcagaagctccaccaactacagaacttcaatacactgatggctgtgataggtgggctgtgtcacagctcaatctcgaggctcaaggagacaagttcgcatgtcccacatgaaatcaataaggttctcggtgagatgactgagctgctgtcctcctccagaaactacgacaattaccggcgagcctatggagagtgcaccgacttcaagatccccattctgggtgtgcatctcaaggacctcatctccctgtatgaagccatgcctgactatctggaggacgggaaagtgaacgtccataagctactggccctatacaatcatatcagtgaattggtccagctgcaagaggtggccccacccttggaggctaacaaggacttggtacacttgctgacgttatccctggatctttactacactgaggatgaaatctatgagctttcctatgcccgggaaccaaggaaccacagagctccaccactaacaccttcaaagccaccagtagtagtggactgggcttctggagtgtctcccaaacctgatccaaaaaccattagcaaacacgtccagaggatggtggattctgtcttcaagaactatgatcacgaccaggatggatacatttctcaggaagaatttgaaaagattgctgcgagttttccattttccttctgtgtgatggacaaagacagggaaggcctcatcagcagggatgagatcacagcctacttcatgagagccagctcaatctattccaagctgggcctgggctttcctcacaacttccaagagaccacctacctgaagcccactttttgtgacaactgtgctggatttctctggggagtgatcaaacaaggatatcgatgtaaagactgcgggatgaactgtcacaaacaatgcaaagatctggttgtgtttgagtgtaagaagcgagccaagaacccagtagctcccacagagaacaacacttctgtggggccagtgtccaacctttgctcattgggagccaaagatctgctccatgcacctgaggaaggaccttttacattccctaatggggaggctgtggaacatggtgaggagagtaaggatcggaccatcatgctgatgggagtgtcctcacagaagatttctcttcggctgaagagggctgttgcccacaaggccacccagactgaatcacagccttggattggcagtgagggcccttcaggtccctttgtgctgtcttccccaaggaagacagcccaggatactctatatgtgcttcccagtcccacctctccatgtcctagcccagtcttggtcagaaagcgggcttttgtcaagtgggagaataaagactccctcataaaatcaaaggaggagctccgtcacctcagactgcctacctaccaagaactggaacaggaaataaatactctgaaagcagataatgatgccctaaagatccaactgaaatatgcacagaagaaaatagaatccctccagcttgaaaaaagcaatcatgtcttagctcaaatggagcagggtgactgttct

SEQ ID NO: 156 - protein, 797 aa, Synthetic construct
MGTLGKAREAPRKPSHGCRAASKARLEAKPANSPFPSHPSLAHITQFRMMVSLGHLAKGASLDDLIDSCIQSFDADGNLCRSNQLLQVMLTMHRIVISSAELLQKVITLYKDALAKNSPGLCLKICYFVRYWITEFWVMFKMDASLTDTMEEFQELVKAKGEELHCRLIDTTQINARDWSRKLTQRIKSNTSKKRKVSLLFDHLEPEELSEHLTYLEFKSFRRISFSDYQNYLVNSCVKENPTMERSIALCNGISQWVQLIVLSRPTPQLRAEVFIKFIQVAQKLHQLQNFNTLMAVIGGLCHSSISRLKETSSHVPHEINKVLGEMTELLSSSRNYDNYRRAYGECTDFKIPILGVHLKDLISLYEAMPDYLEDGKVNVHKLLALYNHISELVQLQEVAPPLEANKDLVHLLTLSLDLYYTEDEIYELSYAREPRNHRAPPLTPSKPPVVVDWASGVSPKPDPKTISKHVQRMVDSVFKNYDHDQDGYISQEEFEKIAASFPFSFCVMDKDREGLISRDEITAYFMRASSIYSKLGLGFPHNFQETTYLKPTFCDNCAGFLWGVIKQGYRCKDCGMNCHKQCKDLVVFECKKRAKNPVAPTENNTSVGPVSNLCSLGAKDLLHAPEEGPFTFPNGEAVEHGEESKDRTIMLMGVSSQKISLRLKRAVAHKATQTESQPWIGSEGPSGPFVLSSPRKTAQDTLYVLPSPTSPCPSPVLVRKRAFVKWENKDSLIKSKEELRHLRLPTYQELEQEINTLKADNDALKIQLKYAQKKIESLQLEKSNHVLAQMEQGDCS

SEQ ID NO: 157 - DNA, 2391 bp, Homo sapiens
  source 1..2391: mol_type = genomic DNA
atgggcaccctgggcaaggcgagagaggctccgcggaaaccttcccatggctgcagagctgcctctaaagcaagactagaggcaaagccagccaacagccccttcccctcccatcccagcttggcccacatcacccagttccgaatgatggtgtctctgggacatttagccaaaggagccagcctggacgatctcattgacagctgcattcaatcttttgatgcagatggaaacctgtgtcgaagtaaccaactgttgcaagtcatgctgaccatgcaccgaattgtcatctcctctgcagaactgctccaaaaagttatcaccctctataaggatgctttggcaaagaattcaccaggactttgcctgaagatctgttattttgtaaggtattggataacagaattctgggtcatgtttaaaatggacgccagcttgacagacactatggaggagtttcaggaactggtgaaagctaagggtgaggagttacattgccgcctgattgacacaactcaaatcaatgcccgtgactggtccaggaaacttactcaaaggataaaatcaaataccagcaagaaacggaaagtctccctgctctttgaccatctggaaccagaagagctatccgagcacctcacctaccttgagttcaagtctttccggaggatatcgttctctgattatcagaattaccttgtaaatagctgtgtgaaggaaaaccccaccatggagcgatctattgctctgtgcaacggcatctcccagtgggtacaactgatggttctcagccgccccacgccgcagctccgagcagaagtcttcatcaagttcatccaggtggctcagaagctccaccaactacagaacttcaatacactgatggctgtgataggtgggctgtgtcacagctcaatctcgaggctcaaggagacaagttcgcatgtcccacatgaaatcaataaggttctcggtgagatgactgagctgctgtcctcctccagaaactacgacaattaccggcgagcctatggagagtgcaccgacttcaagatccccattctgggtgtgcatctcaaggacctcatctccctgtatgaagccatgcctgactatctggaggacgggaaagtgaacgtccataagctactggccctatacaatcatatcagtgaattggtccagctgcaagaggtggccccacccttggaggctaacaaggacttggtacacttgctgacgttatccctggatctttactacactgaggatgaaatctatgagctttcctatgcccgggaaccaaggaaccacagagctccaccactaacaccttcaaagccaccagtagtagtggactgggcttctggagtgtctcccaaacctgatccaaaaaccattagcaaacacgtccagaggatggtggattctgtcttcaagaactatgatcacgaccaggatggatacatttctcaggaagaatttgaaaagattgctgcgagttttccattttccttctgtgtgatggacaaagacagggaaggcctcatcagcagggatgagatcacagcctacttcatgagagccagctcaatctattccaagctgggcctgggctttcctcacaacttccaagagaccacctacctgaagcccactttttgtgacaactgtgctggatttctctggggagtgatcaaacaaggatatcgatgtaaagactgcgggatgaactgtcacaaacaatgcaaagatctggttgtgtttgagtgtaagaagcgagccaagaacccagtagctcccacagagaacaacacttctgtggggccagtgtccaacctttgctcattgggagccaaagatctgctccatgcacctgaggaaggaccttttacattccctaatggggaggctgtggaacatggtgaggagagtaaggatcggaccatcatgctgatgggagtgtcctcacagaagatttctcttcggctgaagagggctgttgcccacaaggccacccagactgaatcacagccttggattggcagtgagggcccttcaggtccctttgtgctgtcttccccaaggaagacagcccaggatactctatatgtgcttcccagtcccacctctccatgtcctagcccagtcttggtcagaaagcgggcttttgtcaagtgggagaataaagactccctcataaaatcaaaggaggagctccgtcacctcagactgcctacctaccaagaactggaacaggaaataaatactctgaaagcagataatgatgccctaaagatccaactgaaatatgcacagaagaaaatagaatccctccagcttgaaaaaagcaatcatgtcttagctcaaatggagcagggtgactgttct

SEQ ID NO: 158 - protein, 797 aa, Homo sapiens
MGTLGKAREAPRKPSHGCRAASKARLEAKPANSPFPSHPSLAHITQFRMMVSLGHLAKGASLDDLIDSCIQSFDADGNLCRSNQLLQVMLTMHRIVISSAELLQKVITLYKDALAKNSPGLCLKICYFVRYWITEFWVMFKMDASLTDTMEEFQELVKAKGEELHCRLIDTTQINARDWSRKLTQRIKSNTSKKRKVSLLFDHLEPEELSEHLTYLEFKSFRRISFSDYQNYLVNSCVKENPTMERSIALCNGISQWVQLMVLSRPTPQLRAEVFIKFIQVAQKLHQLQNFNTLMAVIGGLCHSSISRLKETSSHVPHEINKVLGEMTELLSSSRNYDNYRRAYGECTDFKIPILGVHLKDLISLYEAMPDYLEDGKVNVHKLLALYNHISELVQLQEVAPPLEANKDLVHLLTLSLDLYYTEDEIYELSYAREPRNHRAPPLTPSKPPVVVDWASGVSPKPDPKTISKHVQRMVDSVFKNYDHDQDGYISQEEFEKIAASFPFSFCVMDKDREGLISRDEITAYFMRASSIYSKLGLGFPHNFQETTYLKPTFCDNCAGFLWGVIKQGYRCKDCGMNCHKQCKDLVVFECKKRAKNPVAPTENNTSVGPVSNLCSLGAKDLLHAPEEGPFTFPNGEAVEHGEESKDRTIMLMGVSSQKISLRLKRAVAHKATQTESQPWIGSEGPSGPFVLSSPRKTAQDTLYVLPSPTSPCPSPVLVRKRAFVKWENKDSLIKSKEELRHLRLPTYQELEQEINTLKADNDALKIQLKYAQKKIESLQLEKSNHVLAQMEQGDCS

SEQ ID NO: 159 - DNA, 579 bp, Synthetic construct
  source 1..579: mol_type = other DNA
atggctgcgataagaaagaaactcgtcattgttggcgatggggcttgtgttaaaacttgtcttctcatagttttcagtaaagatcagtttccggaagtttatgtgcctacagtcttcgagaattatgttgccgacatcgaagtggatggcaaacaggtggaacttgctttgtgggataccgcaggacaagaagattacgaccgcctccgacctctctcttaccccgatacggatgtcatacttatgtgcttctccatagacagcccggattcactggaaaatatacccgaaaagtggaccccagaggttaaacatttctgtccgaacgtgccaatcatccttgtcggtaacaaaaaggatcttaggaatgatgagcacactcgccgcgaacttgcgaagatgaagcaagagcctgtcaaacctgaggaaggtagagatatggcaaatcgaataggcgcgtttggctacatggaatgttctgcaaaaacaaaggacggtgtacgagaagtttttgagatggctactcgggcagcgttgcaggctagaagaggtaagaaaaaatcaggctgtttggtgttg

SEQ ID NO: 160 - protein, 193 aa, Synthetic construct
MAAIRKKLVIVGDGACVKTCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLVL

SEQ ID NO: 161 - DNA, 579 bp, Synthetic construct
  source 1..579: mol_type = other DNA
atggctgcgataagaaagaaactcgtcattgttggcgatggggctcgtgggaaaacttgtcttctcatagttttcagtaaagatcagtttccggaagtttatgtgcctacagtcttcgagaattatgttgccgacatcgaagtggatggcaaacaggtggaacttgctttgtgggataccgcaggacaagaagattacgaccgcctccgacctctctcttaccccgatacggatgtcatacttatgtgcttctccatagacagcccggattcactggaaaatatacccgaaaagtggaccccagaggttaaacatttctgtccgaacgtgccaatcatccttgtcggtaacaaaaaggatcttaggaatgatgagcacactcgccgcgaacttgcgaagatgaagcaagagcctgtcaaacctgaggaaggtagagatatggcaaatcgaataggcgcgtttggctacatggaatgttctgcaaaaacaaaggacggtgtacgagaagtttttgagatggctactcgggcagcgttgcaggctagaagaggtaagaaaaaatcaggctgtttggtgttg

SEQ ID NO: 162 - protein, 193 aa, Synthetic construct
MAAIRKKLVIVGDGARGKTCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLVL

SEQ ID NO: 163 - DNA, 579 bp, Homo sapiens
  source 1..579: mol_type = genomic DNA
atggctgcgataagaaagaaactcgtcattgttggcgatggggcttgtgggaaaacttgtcttctcatagttttcagtaaagatcagtttccggaagtttatgtgcctacagtcttcgagaattatgttgccgacatcgaagtggatggcaaacaggtggaacttgctttgtgggataccgcaggacaagaagattacgaccgcctccgacctctctcttaccccgatacggatgtcatacttatgtgcttctccatagacagcccggattcactggaaaatatacccgaaaagtggaccccagaggttaaacatttctgtccgaacgtgccaatcatccttgtcggtaacaaaaaggatcttaggaatgatgagcacactcgccgcgaacttgcgaagatgaagcaagagcctgtcaaacctgaggaaggtagagatatggcaaatcgaataggcgcgtttggctacatggaatgttctgcaaaaacaaaggacggtgtacgagaagtttttgagatggctactcgggcagcgttgcaggctagaagaggtaagaaaaaatcaggctgtttggtgttg

SEQ ID NO: 164 - protein, 193 aa, Homo sapiens
MAAIRKKLVIVGDGACGKTCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLVL

SEQ ID NO: 165 - DNA, 1101 bp, Synthetic construct
  source 1..1101: mol_type = other DNA
atgatgatgatggcgctgagcaagaccttcgggcagaagcccgtgaagttccagctggaggacgacggcgagttctacatgatcggctccgaggtgggaaactacctccgtatgttccgaggttctctgtacaagagatacccctcactctggaggcgactagccactgtggaagagaggaagaaaatagttgcatcgtcacatggtaaaaaaacaaaacctaacactaaggatcacggatacacgactctagccaccagtgtgaccctgttaaaagcctcggaagtggaagagattctggatggcaacgatgagaagtacaaggctgtgtccatcagcacagagccccccacctacctcagggaacagaaggccaagaggaacagccagtgggtacccaccctgcccaacagctcccaccacttagatgccgtgccatgctccacaaccatcaacaggaaccgcatgggccgagacaagaagagaaccttccccctttgctttgatgaccatgacccagctgtgatccatgagaacgcatctcagcccgaggtgctggtccccatccggctggacatggagatcgatgggcagaagctgcgagacgccttcacctggaacatgaatgagaagttgatgacgcctgagatgttttcagaaatcctctgtgacgatctggatttgaacccgctgacgtttgtgccagccatcgcctctgccatcagacagcagatcgagtcctaccccacggacagcatcctggaggaccagtcagaccagcgcgtcatcatcaagctgaacatccatgtgggaaacatttccctggtggaccagtttgagtgggacatgtcagagaaggagaactcaccagagaagtttgccctgaagctgtgctcggagctggggttgggcggggagtttgtcaccaccatcgcatacagcatccggggacagctgagctggcatcagaagacctacgccttcagcgagaaccctctgcccacagtggagattgccatccggaacacgggcgatgcggaccagtggtgcccactgctggagactctgacagacgctgagatggagaagaagatccgcgac

SEQ ID NO: 166 - protein, 367 aa, Synthetic construct
MMMMALSKTFGQKPVKFQLEDDGEFYMIGSEVGNYLRMFRGSLYKRYPSLWRRLATVEERKKIVASSHGKKTKPNTKDHGYTTLATSVTLLKASEVEEILDGNDEKYKAVSISTEPPTYLREQKAKRNSQWVPTLPNSSHHLDAVPCSTTINRNRMGRDKKRTFPLCFDDHDPAVIHENASQPEVLVPIRLDMEIDGQKLRDAFTWNMNEKLMTPEMFSEILCDDLDLNPLTFVPAIASAIRQQIESYPTDSILEDQSDQRVIIKLNIHVGNISLVDQFEWDMSEKENSPEKFALKLCSELGLGGEFVTTIAYSIRGQLSWHQKTYAFSENPLPTVEIAIRNTGDADQWCPLLETLTDAEMEKKIRD

SEQ ID NO: 167 - DNA, 1155 bp, Homo sapiens
  source 1..1155: mol_type = genomic DNA
atgatgatgatggcgctgagcaagaccttcgggcagaagcccgtgaagttccagctggaggacgacggcgagttctacatgatcggctccgaggtgggaaactacctccgtatgttccgaggttctctgtacaagagatacccctcactctggaggcgactagccactgtggaagagaggaagaaaatagttgcatcgtcacatggtaaaaaaacaaaacctaacactaaggatcacggatacacgactctagccaccagtgtgaccctgttaaaagcctcggaagtggaagagattctggatggcaacgatgagaagtacaaggctgtgtccatcagcacagagccccccacctacctcagggaacagaaggccaagaggaacagccagtgggtacccaccctgcccaacagctcccaccacttagatgccgtgccatgctccacaaccatcaacaggaaccgcatgggccgagacaagaagagaaccttccccctttgctttgatgaccatgacccagctgtgatccatgagaacgcatctcagcccgaggtgctggtccccatccggctggacatggagatcgatgggcagaagctgcgagacgccttcacctggaacatgaatgagaagttgatgacgcctgagatgttttcagaaatcctctgtgacgatctggatttgaacccgctgacgtttgtgccagccatcgcctctgccatcagacagcagatcgagtcctaccccacggacagcatcctggaggaccagtcagaccagcgcgtcatcatcaagctgaacatccatgtgggaaacatttccctggtggaccagtttgagtgggacatgtcagagaaggagaactcaccagagaagtttgccctgaagctgtgctcggagctggggttgggcggggagtttgtcaccaccatcgcatacagcatccggggacagctgagctggcatcagaagacctacgccttcagcgagaaccctctgcccacagtggagattgccatccggaacacgggcgatgcggaccagtggtgcccactgctggagactctgacagacgctgagatggagaagaagatccgcgaccaggacaggaacacgaggcggatgaggcgtcttgccaacacggccccggcctgg

SEQ ID NO: 168 - protein, 385 aa, Homo sapiens
MMMMALSKTFGQKPVKFQLEDDGEFYMIGSEVGNYLRMFRGSLYKRYPSLWRRLATVEERKKIVASSHGKKTKPNTKDHGYTTLATSVTLLKASEVEEILDGNDEKYKAVSISTEPPTYLREQKAKRNSQWVPTLPNSSHHLDAVPCSTTINRNRMGRDKKRTFPLCFDDHDPAVIHENASQPEVLVPIRLDMEIDGQKLRDAFTWNMNEKLMTPEMFSEILCDDLDLNPLTFVPAIASAIRQQIESYPTDSILEDQSDQRVIIKLNIHVGNISLVDQFEWDMSEKENSPEKFALKLCSELGLGGEFVTTIAYSIRGQLSWHQKTYAFSENPLPTVEIAIRNTGDADQWCPLLETLTDAEMEKKIRDQDRNTRRMRRLANTAPAW

SEQ ID NO: 169 - DNA, 2310 bp, Synthetic construct
  source 1..2310: mol_type = other DNA
atggcccaatggaatcagctacagcagcttgacacacggtacctggagcagctccatcagctctacagtgacagcttcccaatggagctgcggcagtttctggccccttggattgagagtcaagattgggcatatgcggccagcaaagaatcacatgccactttggtgtttcataatctcctgggagagattgaccagcagtatagccgcttcctgcaagagtcgaatgttctctatcagcacaatctacgaagaatcaagcagtttcttcagagcaggtatcttgagaagccaatggagattgcccggattgtggcccggtgcctgtgggaagaatcacgccttctacagactgcagccactgcggcccagcaagggggccaggccaaccaccccacagcagccgtggtgacggagaagcagcagatgctggagcagcaccttcaggatgtccggaagagagtgcaggatctagaacagaaaatgaaagtggtagagaatctccaggatgactttgatttcaactataaaaccctcaagagtcaaggagacatgcaagatctgaatggaaacaaccagtcagtgaccaggcagaagatgcagcagctggaacagatgctcactgcgctggaccagatgcggagaagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggataacgtcattagcagaatctcaacttcagacccgtcaacaaattaagaaactggaggagttgcagcaaaaagtttcctacaaaggggaccccattgtacagcaccggccgatgctggaggagagaatcgtggagctgtttagaaacttaatgaaaagtgcctttgtggtggagcggcagccctgcatgcccatgcatcctgaccggcccctcgtcatcaagaccggcgtccagttcactactaaagtcaggttgctggtcaaattccctgagttgaattatcagcttaaaattaaagtgtgcattgacaaagactctggggacgttgcagctctcagaggatcccggaaatttaacattctgggcacaaacacaaaagtgatgaacatggaagaatccaacaacggcagcctctctgcagaattcaaacacttgaccctgagggagcagagatgtgggaatgggggccgagccaattgtgatgcttccctgattgtgactgaggagctgcacctgatcacctttgagaccgaggtgtatcaccaaggcctcaagattgacctagagacccactccttgccagttgtggtgatctccaacatctgtcagatgccaaatgcctgggcgtccatcctgtggtacaacatgctgaccaacaatcccaagaatgtaaacttttttaccaagcccccaattggaacctgggatcaagtggccgaggtcctgagctggcagttctcctccaccaccaagcgaggactgagcatcgagcagctgactacactggcagagaaactcttgggacctggtgtgaattattcagggtgtcagatcacatgggctaaattttgcaaagaaaacatggctggcaagggcttctccttctgggtctggctggacaatatcattgaccttgtgaaaaagtacatcctggccctttggaacgaagggtacatcatgggctttatcagtaaggagcgggagcgggccatcttgagcactaagcctccaggcaccttcctgctaagattcagtgaaagcagcaaagaaggaggcgtcactttcacttgggtggagaaggacatcagcggtaagacccagatccagtccgtggaaccatacacaaagcagcagctgaacatcatgtcatttgctgaaatcatcatgggctataagatcatggatgctaccaatatcctggtgtctccactggtctatctctatcctgacattcccaaggaggaggcattcggaaagtattgtcggccagagagccaggagcatcctgaagctgacccaggtagcgctgccccatacctgaagaccaagtttatctgtgtgacaccaacgacctgcagcaataccattgacctgccgatgtccccccgcactttagattcattgatgcagtttggaaataatggtgaaggtgctgaaccctcagcaggagggcagtttgagtccctcacctttgacatggagttgacctcggagtgcgctacctcccccatg

SEQ ID NO: 170 - protein, 770 aa, Synthetic construct
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNIMSFAEIIMGYKIMDATNILVSPLVYLYPDIPKEEAFGKYCRPESQEHPEADPGSAAPYLKTKFICVTPTTCSNTIDLPMSPRTLDSLMQFGNNGEGAEPSAGGQFESLTFDMELTSECATSPM

SEQ ID NO: 171 - DNA, 2310 bp, Synthetic construct
  source 1..2310: mol_type = other DNA
atggcccaatggaatcagctacagcagcttgacacacggtacctggagcagctccatcagctctacagtgacagcttcccaatggagctgcggcagtttctggccccttggattgagagtcaagattgggcatatgcggccagcaaagaatcacatgccactttggtgtttcataatctcctgggagagattgaccagcagtatagccgcttcctgcaagagtcgaatgttctctatcagcacaatctacgaagaatcaagcagtttcttcagagcaggtatcttgagaagccaatggagattgcccggattgtggcccggtgcctgtgggaagaatcacgccttctacagactgcagccactgcggcccagcaagggggccaggccaaccaccccacagcagccgtggtgacggagaagcagcagatgctggagcagcaccttcaggatgtccggaagagagtgcaggatctagaacagaaaatgaaagtggtagagaatctccaggatgactttgatttcaactataaaaccctcaagagtcaaggagacatgcaagatctgaatggaaacaaccagtcagtgaccaggcagaagatgcagcagctggaacagatgctcactgcgctggaccagatgcggagaagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggataacgtcattagcagaatctcaacttcagacccgtcaacaaattaagaaactggaggagttgcagcaaaaagtttcctacaaaggggaccccattgtacagcaccggccgatgctggaggagagaatcgtggagctgtttagaaacttaatgaaaagtgcctttgtggtggagcggcagccctgcatgcccatgcatcctgaccggcccctcgtcatcaagaccggcgtccagttcactactaaagtcaggttgctggtcaaattccctgagttgaattatcagcttaaaattaaagtgtgcattgacaaagactctggggacgttgcagctctcagaggatcccggaaatttaacattctgggcacaaacacaaaagtgatgaacatggaagaatccaacaacggcagcctctctgcagaattcaaacacttgaccctgagggagcagagatgtgggaatgggggccgagccaattgtgatgcttccctgattgtgactgaggagctgcacctgatcacctttgagaccgaggtgtatcaccaaggcctcaagattgacctagagacccactccttgccagttgtggtgatctccaacatctgtcagatgccaaatgcctgggcgtccatcctgtggtacaacatgctgaccaacaatcccaagaatgtaaacttttttaccaagcccccaattggaacctgggatcaagtggccgaggtcctgagctggcagttctcctccaccaccaagcgaggactgagcatcgagcagctgactacactggcagagaaactcttgggacctggtgtgaattattcagggtgtcagatcacatgggctaaattttgcaaagaaaacatggctggcaagggcttctccttctgggtctggctggacaatatcattgaccttgtgaaaaagtacatcctggccctttggaacgaagggtacatcatgggctttatcagtaaggagcgggagcgggccatcttgagcactaagcctccaggcaccttcctgctaagattcagtgaaagcagcaaagaaggacgcgtcactttcacttgggtggagaaggacatcagcggtaagacccagatccagtccgtggaaccatacacaaagcagcagctgaacaacatgtcatttgctgaaatcatcatgggctataagatcatggatgctaccaatatcctggtgtctccactggtctatctctatcctgacattcccaaggaggaggcattcggaaagtattgtcggccagagagccaggagcatcctgaagctgacccaggtagcgctgccccatacctgaagaccaagtttatctgtgtgacaccaacgacctgcagcaataccattgacctgccgatgtccccccgcactttagattcattgatgcagtttggaaataatggtgaaggtgctgaaccctcagcaggagggcagtttgagtccctcacctttgacatggagttgacctcggagtgcgctacctcccccatg

SEQ ID NO: 172 - protein, 770 aa, Synthetic construct
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGRVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTPTTCSNTIDLPMSPRTLDSLMQFGNNGEGAEPSAGGQFESLTFDMELTSECATSPM

SEQ ID NO: 173 - DNA, 2310 bp, Synthetic construct
  source 1..2310: mol_type = other DNA
atggcccaatggaatcagctacagcagcttgacacacggtacctggagcagctccatcagctctacagtgacagcttcccaatggagctgcggcagtttctggccccttggattgagagtcaagattgggcatatgcggccagcaaagaatcacatgccactttggtgtttcataatctcctgggagagattgaccagcagtatagccgcttcctgcaagagtcgaatgttctctatcagcacaatctacgaagaatcaagcagtttcttcagagcaggtatcttgagaagccaatggagattgcccggattgtggcccggtgcctgtgggaagaatcacgccttctacagactgcagccactgcggcccagcaagggggccaggccaaccaccccacagcagccgtggtgacggagaagcagcagatgctggagcagcaccttcaggatgtccggaagagagtgcaggatctagaacagaaaatgaaagtggtagagaatctccaggatgactttgatttcaactataaaaccctcaagagtcaaggagacatgcaagatctgaatggaaacaaccagtcagtgaccaggcagaagatgcagcagctggaacagatgctcactgcgctggaccagatgcggagaagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggataacgtcattagcagaatctcaacttcagacccgtcaacaaattaagaaactggaggagttgcagcaaaaagtttcctacaaaggggaccccattgtacagcaccggccgatgctggaggagagaatcgtggagctgtttagaaacttaatgaaaagtgcctttgtggtggagcggcagccctgcatgcccatgcatcctgaccggcccctcgtcatcaagaccggcgtccagttcactactaaagtcaggttgctggtcaaattccctgagttgaattatcagcttaaaattaaagtgtgcattgacaaagactctggggacgttgcagctctcagaggatcccggaaatttaacattctgggcacaaacacaaaagtgatgaacatggaagaatccaacaacggcagcctctctgcagaattcaaacacttgaccctgagggagcagagatgtgggaatgggggccgagccaattgtgatgcttccctgattgtgactgaggagctgcacctgatcacctttgagaccgaggtgtatcaccaaggcctcaagattgacctagagacccactccttgccagttgtggtgatctccaacatctgtcagatgccaaatgcctgggcgtccatcctgtggtacaacatgctgaccaacaatcccaagaatgtaaacttttttaccaagcccccaattggaacctgggatcaagtggccgaggtcctgagctggcagttctcctccaccaccaagcgaggactgagcatcgagcagctgactacactggcagagaaactcttgggacctggtgtgaattattcagggtgtcagatcacatgggctaaattttgcaaagaaaacatggctggcaagggcttctccttctgggtctggctggacaatatcattgaccttgtgaaaaagtacatcctggccctttggaacgaagggtacatcatgggctttatcagtaaggagcgggagcgggccatcttgagcactaagcctccaggcaccttcctgctaagattcagtgaaagcagcaaagaaggaggcgtcactttcacttgggtggagaaggacatcagcggtaagacccagatccagtccgtggaaccatacacaaagcagcagctgaacaacatgtcatttgctgaaatcatcatgggctataagatcatgattgctaccaatatcctggtgtctccactggtctatctctatcctgacattcccaaggaggaggcattcggaaagtattgtcggccagagagccaggagcatcctgaagctgacccaggtagcgctgccccatacctgaagaccaagtttatctgtgtgacaccaacgacctgcagcaataccattgacctgccgatgtccccccgcactttagattcattgatgcagtttggaaataatggtgaaggtgctgaaccctcagcaggagggcagtttgagtccctcacctttgacatggagttgacctcggagtgcgctacctcccccatg

SEQ ID NO: 174 - protein, 770 aa, Synthetic construct
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMIATNILVSPLVYLYPDIPKEEAFGKYCRPESQEHPEADPGSAAPYLKTKFICVTPTTCSNTIDLPMSPRTLDSLMQFGNNGEGAEPSAGGQFESLTFDMELTSECATSPM

SEQ ID NO: 175 - DNA, 2310 bp, Homo sapiens
  source 1..2310: mol_type = genomic DNA
atggcccaatggaatcagctacagcagcttgacacacggtacctggagcagctccatcagctctacagtgacagcttcccaatggagctgcggcagtttctggccccttggattgagagtcaagattgggcatatgcggccagcaaagaatcacatgccactttggtgtttcataatctcctgggagagattgaccagcagtatagccgcttcctgcaagagtcgaatgttctctatcagcacaatctacgaagaatcaagcagtttcttcagagcaggtatcttgagaagccaatggagattgcccggattgtggcccggtgcctgtgggaagaatcacgccttctacagactgcagccactgcggcccagcaagggggccaggccaaccaccccacagcagccgtggtgacggagaagcagcagatgctggagcagcaccttcaggatgtccggaagagagtgcaggatctagaacagaaaatgaaagtggtagagaatctccaggatgactttgatttcaactataaaaccctcaagagtcaaggagacatgcaagatctgaatggaaacaaccagtcagtgaccaggcagaagatgcagcagctggaacagatgctcactgcgctggaccagatgcggagaagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggataacgtcattagcagaatctcaacttcagacccgtcaacaaattaagaaactggaggagttgcagcaaaaagtttcctacaaaggggaccccattgtacagcaccggccgatgctggaggagagaatcgtggagctgtttagaaacttaatgaaaagtgcctttgtggtggagcggcagccctgcatgcccatgcatcctgaccggcccctcgtcatcaagaccggcgtccagttcactactaaagtcaggttgctggtcaaattccctgagttgaattatcagcttaaaattaaagtgtgcattgacaaagactctggggacgttgcagctctcagaggatcccggaaatttaacattctgggcacaaacacaaaagtgatgaacatggaagaatccaacaacggcagcctctctgcagaattcaaacacttgaccctgagggagcagagatgtgggaatgggggccgagccaattgtgatgcttccctgattgtgactgaggagctgcacctgatcacctttgagaccgaggtgtatcaccaaggcctcaagattgacctagagacccactccttgccagttgtggtgatctccaacatctgtcagatgccaaatgcctgggcgtccatcctgtggtacaacatgctgaccaacaatcccaagaatgtaaacttttttaccaagcccccaattggaacctgggatcaagtggccgaggtcctgagctggcagttctcctccaccaccaagcgaggactgagcatcgagcagctgactacactggcagagaaactcttgggacctggtgtgaattattcagggtgtcagatcacatgggctaaattttgcaaagaaaacatggctggcaagggcttctccttctgggtctggctggacaatatcattgaccttgtgaaaaagtacatcctggccctttggaacgaagggtacatcatgggctttatcagtaaggagcgggagcgggccatcttgagcactaagcctccaggcaccttcctgctaagattcagtgaaagcagcaaagaaggaggcgtcactttcacttgggtggagaaggacatcagcggtaagacccagatccagtccgtggaaccatacacaaagcagcagctgaacaacatgtcatttgctgaaatcatcatgggctataagatcatggatgctaccaatatcctggtgtctccactggtctatctctatcctgacattcccaaggaggaggcattcggaaagtattgtcggccagagagccaggagcatcctgaagctgacccaggtagcgctgccccatacctgaagaccaagtttatctgtgtgacaccaacgacctgcagcaataccattgacctgccgatgtccccccgcactttagattcattgatgcagtttggaaataatggtgaaggtgctgaaccctcagcaggagggcagtttgagtccctcacctttgacatggagttgacctcggagtgcgctacctcccccatg

SEQ ID NO: 176 - protein, 770 aa, Homo sapiens
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTPTTCSNTIDLPMSPRTLDSLMQFGNNGEGAEPSAGGQFESLTFDMELTSECATSPM

SEQ ID NO: 177 - DNA, 2364 bp, Synthetic construct
  source 1..2364: mol_type = other DNA
atggctgtgtggatacaagctcagcagctccaaggagaagcccttcatcagatgcaagcgttatatggccagcattttcccattgaggtgcggcattatttatcccagtggattgaaagccaagcatgggactcagtagatcttgataatccacaggagaacattaaggccacccagctcctggagggcctggtgcaggagctgcagaagaaggcagagcaccaggtgggggaagatgggtttttactgaagatcaagctggggcactatgccacacagctccagaacacgtatgaccgctgccccatggagctggtccgctgcatccgccatatattgtacaatgaacagaggttggtccgagaagccaacaatggtagctctccagctggaagccttgctgatgccatgtcccagaaacacctccagatcaaccagacgtttgaggagctgcgactggtcacgcaggacacagagaatgagttaaaaaagctgcagcagactcaggagtacttcatcatccagtaccaggagagcctgaggatccaagctcagtttggcccgctggcccagctgagcccccaggagcgtctgagccgggagacggccctccagcagaagcaggtgtctctggaggcctggttgcagcgtgaggcacagacactgcagcagtaccgcgtggagctggccgagaagcaccagaagaccctgcagctgctgcggaagcagcagaccatcatcctggatgacgagctgatccagtggaagcggcggcagcagctggccgggaacggcgggccccccgagggcagcctggacgtgctacagtcctggtgtgagaagttggccgagatcatctggcagaaccggcagcagatccgcagggctgagcacctctgccagcagctgcccatccccggcccagtggaggagatgctggccgaggtcaacgccaccatcacggacattatctcagccctggtgaccagcacgttcatcattgagaagcagcctcctcaggtcctgaagacccagaccaagtttgcagccactgtgcgcctgctggtgggcgggaagctgaacgtgcacatgaaccccccccaggtgaaggccaccatcatcagtgagcagcaggccaagtctctgctcaagaacgagaacacccgcaatgattacagtggcgagatcttgaacaactgctgcgtcatggagtaccaccaagccacaggcacccttagtgcccacttcaggaatatgtccctgaaacgaattaagaggtcagaccgtcgtggggcagagtcggtgacagaagaaaaatttacaatcctgtttgaatcccagttcagtgttggtggaaatgagctggtttttcaagtcaagaccctgtccctgccagtggtggtgatcgttcatggcagccaggacaacaatgcgacggccactgttctctgggacaatgcttttgcagagcctggcagggtgccatttgccgtgcctgacaaagtgctgtggccacagctgtgtgaggcgctcaacatgaaattcaaggccgaagtgcagagcaaccggggcctgaccaaggagaacctcgtgttcctggcgcagaaactgttcaacaacagcagcagccacctggaggactacagtggcctgtctgtgtcctggtcccagttcaacagggagaatttaccaggacggaattacactttctggcaatggtttgacggtgtgatggaagtgttaaaaaaacatctcaagcctcattggaatgatggggccattttggggtttgtaaacaagcaacaggcccatgacctactcattaacaagccagatgggaccttcctcctgagattcagtgactcagaaattggcggcatcagcattgcttggaagtttgattctcaggaaagaatgttttggaatctgatgccttttaccaccagagacttctccattcggtccctagccgaccgcttgggagacttgaattaccttatctacgtgtttcctgatcggccaaaagatgaagtatactccaaatactacacaccagttccctgcgagtctgctactgctaaagctgttgatggatacgtgaagccacagatcaagcaagtggtccctgagtttgtgaacgcatctgcagatgccgggggcggcagcgccacgtacatggaccaggccccctccccagctgtgtgtccccaggctcactataacatgtacccacagaaccctgactcagtccttgacaccgatggggacttcgatctggaggacacaatggacgtagcgcggcgtgtggaggagctcctgggccggccaatggacagtcagtggatcccgcacgcacaatcgttg

SEQ ID NO: 178 - protein, 788 aa, Synthetic construct
MAVWIQAQQLQGEALHQMQALYGQHFPIEVRHYLSQWIESQAWDSVDLDNPQENIKATQLLEGLVQELQKKAEHQVGEDGFLLKIKLGHYATQLQNTYDRCPMELVRCIRHILYNEQRLVREANNGSSPAGSLADAMSQKHLQINQTFEELRLVTQDTENELKKLQQTQEYFIIQYQESLRIQAQFGPLAQLSPQERLSRETALQQKQVSLEAWLQREAQTLQQYRVELAEKHQKTLQLLRKQQTIILDDELIQWKRRQQLAGNGGPPEGSLDVLQSWCEKLAEIIWQNRQQIRRAEHLCQQLPIPGPVEEMLAEVNATITDIISALVTSTFIIEKQPPQVLKTQTKFAATVRLLVGGKLNVHMNPPQVKATIISEQQAKSLLKNENTRNDYSGEILNNCCVMEYHQATGTLSAHFRNMSLKRIKRSDRRGAESVTEEKFTILFESQFSVGGNELVFQVKTLSLPVVVIVHGSQDNNATATVLWDNAFAEPGRVPFAVPDKVLWPQLCEALNMKFKAEVQSNRGLTKENLVFLAQKLFNNSSSHLEDYSGLSVSWSQFNRENLPGRNYTFWQWFDGVMEVLKKHLKPHWNDGAILGFVNKQQAHDLLINKPDGTFLLRFSDSEIGGISIAWKFDSQERMFWNLMPFTTRDFSIRSLADRLGDLNYLIYVFPDRPKDEVYSKYYTPVPCESATAKAVDGYVKPQIKQVVPEFVNASADAGGGSATYMDQAPSPAVCPQAHYNMYPQNPDSVLDTDGDFDLEDTMDVARRVEELLGRPMDSQWIPHAQSL

SEQ ID NO: 179 - DNA, 2364 bp, Synthetic construct
  source 1..2364: mol_type = other DNA
atggctgtgtggatacaagctcagcagctccaaggagaagcccttcatcagatgcaagcgttatatggccagcattttcccattgaggtgcggcattatttatcccagtggattgaaagccaagcatgggactcagtagatcttgataatccacaggagaacattaaggccacccagctcctggagggcctggtgcaggagctgcagaagaaggcagagcaccaggtgggggaagatgggtttttactgaagatcaagctggggcactatgccacacagctccagaacacgtatgaccgctgccccatggagctggtccgctgcatccgccatatattgtacaatgaacagaggttggtccgagaagccaacaatggtagctctccagctggaagccttgctgatgccatgtcccagaaacacctccagatcaaccagacgtttgaggagctgcgactggtcacgcaggacacagagaatgagttaaaaaagctgcagcagactcaggagtacttcatcatccagtaccaggagagcctgaggatccaagctcagtttggcccgctggcccagctgagcccccaggagcgtctgagccgggagacggccctccagcagaagcaggtgtctctggaggcctggttgcagcgtgaggcacagacactgcagcagtaccgcgtggagctggccgagaagcaccagaagaccctgcagctgctgcggaagcagcagaccatcatcctggatgacgagctgatccagtggaagcggcggcagcagctggccgggaacggcgggccccccgagggcagcctggacgtgctacagtcctggtgtgagaagttggccgagatcatctggcagaaccggcagcagatccgcagggctgagcacctctgccagcagctgcccatccccggcccagtggaggagatgctggccgaggtcaacgccaccatcacggacattatctcagccctggtgaccagcacgttcatcattgagaagcagcctcctcaggtcctgaagacccagaccaagtttgcagccactgtgcgcctgctggtgggcgggaagctgaacgtgcacatgaaccccccccaggtgaaggccaccatcatcagtgagcagcaggccaagtctctgctcaagaacgagaacacccgcaatgattacagtggcgagatcttgaacaactgctgcgtcatggagtaccaccaagccacaggcacccttagtgcccacttcaggaatatgtccctgaaacgaattaagaggtcagaccgtcgtggggcagagtcggtgacagaagaaaaatttacaatcctgtttgaatcccagttcagtgttggtggaaatgagctggtttttcaagtcaagaccctgtccctgccagtggtggtgatcgttcatggcagccaggacaacaatgcgacggccactgttctctgggacaatgcttttgcagagcctggcagggtgccatttgccgtgcctgacaaagtgctgtggccacagctgtgtgaggcgctcaacatgaaattcaaggccgaagtgcagagcaaccggggcctgaccaaggagaacctcgtgttcctggcgcagaaactgttcaacaacagcagcagccacctggaggactacagtggcctgtctgtgtcctggtcccagttcaacagggagaatttaccaggacggaattacactttctggcaatggtttgacggtgtgatggaagtgttaaaaaaacatctcaagcctcattggaatgatggggccattttggggtttgtaaacaagcaacaggcccatgacctactcattaacaagccagatgggaccttcctcctgagattcagtgactcagaaattggcggcatcaccattgcttggaagtttgattctcaggaaagaatgttttggaatctgatgccttttaccaccagagacttctccattcggtccctagccgaccgcttgggagacttgaatttccttatctacgtgtttcctgatcggccaaaagatgaagtatactccaaatactacacaccagttccctgcgagtctgctactgctaaagctgttgatggatacgtgaagccacagatcaagcaagtggtccctgagtttgtgaacgcatctgcagatgccgggggcggcagcgccacgtacatggaccaggccccctccccagctgtgtgtccccaggctcactataacatgtacccacagaaccctgactcagtccttgacaccgatggggacttcgatctggaggacacaatggacgtagcgcggcgtgtggaggagctcctgggccggccaatggacagtcagtggatcccgcacgcacaatcgttg

SEQ ID NO: 180 - protein, 788 aa, Synthetic construct
MAVWIQAQQLQGEALHQMQALYGQHFPIEVRHYLSQWIESQAWDSVDLDNPQENIKATQLLEGLVQELQKKAEHQVGEDGFLLKIKLGHYATQLQNTYDRCPMELVRCIRHILYNEQRLVREANNGSSPAGSLADAMSQKHLQINQTFEELRLVTQDTENELKKLQQTQEYFIIQYQESLRIQAQFGPLAQLSPQERLSRETALQQKQVSLEAWLQREAQTLQQYRVELAEKHQKTLQLLRKQQTIILDDELIQWKRRQQLAGNGGPPEGSLDVLQSWCEKLAEIIWQNRQQIRRAEHLCQQLPIPGPVEEMLAEVNATITDIISALVTSTFIIEKQPPQVLKTQTKFAATVRLLVGGKLNVHMNPPQVKATIISEQQAKSLLKNENTRNDYSGEILNNCCVMEYHQATGTLSAHFRNMSLKRIKRSDRRGAESVTEEKFTILFESQFSVGGNELVFQVKTLSLPVVVIVHGSQDNNATATVLWDNAFAEPGRVPFAVPDKVLWPQLCEALNMKFKAEVQSNRGLTKENLVFLAQKLFNNSSSHLEDYSGLSVSWSQFNRENLPGRNYTFWQWFDGVMEVLKKHLKPHWNDGAILGFVNKQQAHDLLINKPDGTFLLRFSDSEIGGITIAWKFDSQERMFWNLMPFTTRDFSIRSLADRLGDLNFLIYVFPDRPKDEVYSKYYTPVPCESATAKAVDGYVKPQIKQVVPEFVNASADAGGGSATYMDQAPSPAVCPQAHYNMYPQNPDSVLDTDGDFDLEDTMDVARRVEELLGRPMDSQWIPHAQSL

SEQ ID NO: 181 - DNA, 2364 bp, Homo sapiens
  source 1..2364: mol_type = genomic DNA
atggctgtgtggatacaagctcagcagctccaaggagaagcccttcatcagatgcaagcgttatatggccagcattttcccattgaggtgcggcattatttatcccagtggattgaaagccaagcatgggactcagtagatcttgataatccacaggagaacattaaggccacccagctcctggagggcctggtgcaggagctgcagaagaaggcagagcaccaggtgggggaagatgggtttttactgaagatcaagctggggcactatgccacacagctccagaacacgtatgaccgctgccccatggagctggtccgctgcatccgccatatattgtacaatgaacagaggttggtccgagaagccaacaatggtagctctccagctggaagccttgctgatgccatgtcccagaaacacctccagatcaaccagacgtttgaggagctgcgactggtcacgcaggacacagagaatgagttaaaaaagctgcagcagactcaggagtacttcatcatccagtaccaggagagcctgaggatccaagctcagtttggcccgctggcccagctgagcccccaggagcgtctgagccgggagacggccctccagcagaagcaggtgtctctggaggcctggttgcagcgtgaggcacagacactgcagcagtaccgcgtggagctggccgagaagcaccagaagaccctgcagctgctgcggaagcagcagaccatcatcctggatgacgagctgatccagtggaagcggcggcagcagctggccgggaacggcgggccccccgagggcagcctggacgtgctacagtcctggtgtgagaagttggccgagatcatctggcagaaccggcagcagatccgcagggctgagcacctctgccagcagctgcccatccccggcccagtggaggagatgctggccgaggtcaacgccaccatcacggacattatctcagccctggtgaccagcacgttcatcattgagaagcagcctcctcaggtcctgaagacccagaccaagtttgcagccactgtgcgcctgctggtgggcgggaagctgaacgtgcacatgaaccccccccaggtgaaggccaccatcatcagtgagcagcaggccaagtctctgctcaagaacgagaacacccgcaatgattacagtggcgagatcttgaacaactgctgcgtcatggagtaccaccaagccacaggcacccttagtgcccacttcaggaatatgtccctgaaacgaattaagaggtcagaccgtcgtggggcagagtcggtgacagaagaaaaatttacaatcctgtttgaatcccagttcagtgttggtggaaatgagctggtttttcaagtcaagaccctgtccctgccagtggtggtgatcgttcatggcagccaggacaacaatgcgacggccactgttctctgggacaatgcttttgcagagcctggcagggtgccatttgccgtgcctgacaaagtgctgtggccacagctgtgtgaggcgctcaacatgaaattcaaggccgaagtgcagagcaaccggggcctgaccaaggagaacctcgtgttcctggcgcagaaactgttcaacaacagcagcagccacctggaggactacagtggcctgtctgtgtcctggtcccagttcaacagggagaatttaccaggacggaattacactttctggcaatggtttgacggtgtgatggaagtgttaaaaaaacatctcaagcctcattggaatgatggggccattttggggtttgtaaacaagcaacaggcccatgacctactcattaacaagccagatgggaccttcctcctgagattcagtgactcagaaattggcggcatcaccattgcttggaagtttgattctcaggaaagaatgttttggaatctgatgccttttaccaccagagacttctccattcggtccctagccgaccgcttgggagacttgaattaccttatctacgtgtttcctgatcggccaaaagatgaagtatactccaaatactacacaccagttccctgcgagtctgctactgctaaagctgttgatggatacgtgaagccacagatcaagcaagtggtccctgagtttgtgaacgcatctgcagatgccgggggcggcagcgccacgtacatggaccaggccccctccccagctgtgtgtccccaggctcactataacatgtacccacagaaccctgactcagtccttgacaccgatggggacttcgatctggaggacacaatggacgtagcgcggcgtgtggaggagctcctgggccggccaatggacagtcagtggatcccgcacgcacaatcgttg

SEQ ID NO: 182 - protein, 788 aa, Homo sapiens
MAVWIQAQQLQGEALHQMQALYGQHFPIEVRHYLSQWIESQAWDSVDLDNPQENIKATQLLEGLVQELQKKAEHQVGEDGFLLKIKLGHYATQLQNTYDRCPMELVRCIRHILYNEQRLVREANNGSSPAGSLADAMSQKHLQINQTFEELRLVTQDTENELKKLQQTQEYFIIQYQESLRIQAQFGPLAQLSPQERLSRETALQQKQVSLEAWLQREAQTLQQYRVELAEKHQKTLQLLRKQQTIILDDELIQWKRRQQLAGNGGPPEGSLDVLQSWCEKLAEIIWQNRQQIRRAEHLCQQLPIPGPVEEMLAEVNATITDIISALVTSTFIIEKQPPQVLKTQTKFAATVRLLVGGKLNVHMNPPQVKATIISEQQAKSLLKNENTRNDYSGEILNNCCVMEYHQATGTLSAHFRNMSLKRIKRSDRRGAESVTEEKFTILFESQFSVGGNELVFQVKTLSLPVVVIVHGSQDNNATATVLWDNAFAEPGRVPFAVPDKVLWPQLCEALNMKFKAEVQSNRGLTKENLVFLAQKLFNNSSSHLEDYSGLSVSWSQFNRENLPGRNYTFWQWFDGVMEVLKKHLKPHWNDGAILGFVNKQQAHDLLINKPDGTFLLRFSDSEIGGITIAWKFDSQERMFWNLMPFTTRDFSIRSLADRLGDLNYLIYVFPDRPKDEVYSKYYTPVPCESATAKAVDGYVKPQIKQVVPEFVNASADAGGGSATYMDQAPSPAVCPQAHYNMYPQNPDSVLDTDGDFDLEDTMDVARRVEELLGRPMDSQWIPHAQSL

SEQ ID NO: 183 - DNA, 1542 bp, Homo sapiens
  source 1..1542: mol_type = genomic DNA
atgagtataagcagtgatgaggtcaacttcttggtatatagatacttgcaagagtcaggattttctcattcagcatttacctttggtatagaaagccatatcagtcagtccaatataaatggtgccctcgtcccacccgctgcattgatttctatcatccagaaaggtctacagtatgtagaagcagaagttagtattaatgaggatggtaccttgtttgatggtcgaccaatagagtctctgtccctgatagatgccgtaatgcctgatgtagtacaaacaagacaacaagcttatagagataagcttgcacagcaacaggcagcagctgctgcagctgccgcagctgcagccagccaacaaggatctgcaaaaaatggagaaaacacagcaaatggggaggagaatggagcacatactatagcaaataatcatactgatatgatggaagtggatggggatgttgaaatccctcctaataaagctgttgtgttgcggggccatgaatctgaagtttttatctgtgcctggaaccctgttagtgatctcctagcatcagggtctggagactcaacagcaagaatatggaatcttagtgagaacagcaccagtggctctacacagttagtacttagacattgtatacgagaaggagggcaagatgttccaagcaacaaggatgtcacatctctagattggaatagtgaaggtacacttctagcaactggttcctatgatgggtttgccagaatatggactaaagatggtaaccttgctagcaccttagggcagcataaaggccctatatttgcattaaaatggaataagaaaggaaatttcatcctaagtgctggagtagacaagactacaattatttgggacgcacatactggtgaagccaagcaacagtttccttttcattcagcaccagcattggatgttgattggcagagcaacaacacctttgcttcttgtagtacagatatgtgcattcatgtctgtaaattaggacaagacagacctattaaaacattccaaggacatacgaatgaagtaaatgctatcaaatgggacccaactggcaatctcttggcctcctgttctgacgacatgactttaaagatatggagtatgaaacaagacaattgtgtccatgatttgcaagcacataataaagaaatttatactatcaaatggagtccaacaggaccagggactaataatccaaatgccaaccttatgttagcaagtgcatcctttgattctactgttaggttatgggatgtagaccgagggatatgcatccatactttgacaaaacaccaagagcctgtgtacagtgtagctttcagtcctgatggcaggtatctggcaagtggttcttttgacaaatgtgtacacatctggaacacgcagacaggtgctctagttcacagctataggggaacaggtggaatatttgaagtttgctggaatgcagcaggagacaaagttggagccagtgcatcagatggttcagtttgtgtattagaccttcggaaa

SEQ ID NO: 184 - protein, 514 aa, Homo sapiens
MSISSDEVNFLVYRYLQESGFSHSAFTFGIESHISQSNINGALVPPAALISIIQKGLQYVEAEVSINEDGTLFDGRPIESLSLIDAVMPDVVQTRQQAYRDKLAQQQAAAAAAAAAAASQQGSAKNGENTANGEENGAHTIANNHTDMMEVDGDVEIPPNKAVVLRGHESEVFICAWNPVSDLLASGSGDSTARIWNLSENSTSGSTQLVLRHCIREGGQDVPSNKDVTSLDWNSEGTLLATGSYDGFARIWTKDGNLASTLGQHKGPIFALKWNKKGNFILSAGVDKTTIIWDAHTGEAKQQFPFHSAPALDVDWQSNNTFASCSTDMCIHVCKLGQDRPIKTFQGHTNEVNAIKWDPTGNLLASCSDDMTLKIWSMKQDNCVHDLQAHNKEIYTIKWSPTGPGTNNPNANLMLASASFDSTVRLWDVDRGICIHTLTKHQEPVYSVAFSPDGRYLASGSFDKCVHIWNTQTGALVHSYRGTGGIFEVCWNAAGDKVGASASDGSVCVLDLRK

SEQ ID NO: 185 - DNA, 1542 bp, Synthetic construct
  source 1..1542: mol_type = other DNA
atgagtataagcagtgatgaggtcaacttcttggtatatagatacttgcaagagtcaggattttctcattcagcatttacctttggtatagaaagccatatcagtcagtccaatataaatggtgccctcgtcccacccgctgcattgatttctatcatccagaaaggtctacagtatgtagaagcagaagttagtattaatgaggatggtaccttgtttgatggtcgaccaatagagtctctgtccctgatagatgccgtaatgcctgatgtagtacaaacaagacaacaagcttatagagataagcttgcacagcaacaggcagcagctgctgcagctgccgcagctgcagccagccaacaaggatctgcaaaaaatggagaaaacacagcaaatggggaggagaatggagcacatactatagcaaataatcatactgatatgatggaagtggatggggatgttgaaatccctcctaataaagctgttgtgttgcggggccatgaatctgaagtttttatctgtgcctggaaccctgttagtgatctcctagcatcagggtctggagactcaacagcaagaatatggaatcttagtgagaacagcaccagtggctctacacagttagtacttagacattgtatacgagaaggagggcaagatgttccaagcaacaaggatgtcacatctctagattggaatagtgaaggtacacttctagcaactggttcctatgatgggtttgccagaatatggactaaagatggtaaccttgctagcaccttagggcagcataaaggccctatatttgcattaaaatggaataagaaaggaaatttcatcctaagtgctggagtagacaagactacaattatttgggacgcacatactggtgaagccaagcaacagtttccttttcgttcagcaccagcattggatgttgattggcagagcaacaacacctttgcttcttgtagtacagatatgtgcattcatgtctgtaaattaggacaagacagacctattaaaacattccaaggacatacgaatgaagtaaatgctatcaaatgggacccaactggcaatctcttggcctcctgttctgacgacatgactttaaagatatggagtatgaaacaagacaattgtgtccatgatttgcaagcacataataaagaaatttatactatcaaatggagtccaacaggaccagggactaataatccaaatgccaaccttatgttagcaagtgcatcctttgattctactgttaggttatgggatgtagaccgagggatatgcatccatactttgacaaaacaccaagagcctgtgtacagtgtagctttcagtcctgatggcaggtatctggcaagtggttcttttgacaaatgtgtacacatctggaacacgcagacaggtgctctagttcacagctataggggaacaggtggaatatttgaagtttgctggaatgcagcaggagacaaagttggagccagtgcatcagatggttcagtttgtgtattagaccttcggaaa

SEQ ID NO: 186 - protein, 514 aa, Synthetic construct
MSISSDEVNFLVYRYLQESGFSHSAFTFGIESHISQSNINGALVPPAALISIIQKGLQYVEAEVSINEDGTLFDGRPIESLSLIDAVMPDVVQTRQQAYRDKLAQQQAAAAAAAAAAASQQGSAKNGENTANGEENGAHTIANNHTDMMEVDGDVEIPPNKAVVLRGHESEVFICAWNPVSDLLASGSGDSTARIWNLSENSTSGSTQLVLRHCIREGGQDVPSNKDVTSLDWNSEGTLLATGSYDGFARIWTKDGNLASTLGQHKGPIFALKWNKKGNFILSAGVDKTTIIWDAHTGEAKQQFPFRSAPALDVDWQSNNTFASCSTDMCIHVCKLGQDRPIKTFQGHTNEVNAIKWDPTGNLLASCSDDMTLKIWSMKQDNCVHDLQAHNKEIYTIKWSPTGPGTNNPNANLMLASASFDSTVRLWDVDRGICIHTLTKHQEPVYSVAFSPDGRYLASGSFDKCVHIWNTQTGALVHSYRGTGGIFEVCWNAAGDKVGASASDGSVCVLDLRK

SEQ ID NO: 187 - DNA, 1383 bp, Synthetic construct
  source 1..1383: mol_type = other DNA
atggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacttcgctcttccagttggactgattgtgggtgtgacagccttgggtctactaataataggagtggtgaactgtgtcatcatgacccaggtgaaaaagaagcccttgtgcctgcagagagaagccaaggtgcctcacttgcctgccgataaggcccggggtacacagggccccgagcagcagcacctgctgatcacagcgccgagctccagcagcagctccctggagagctcggccagtgcgttggacagaagggcgcccactcggaaccagccacaggcaccaggcgtggaggccagtggggccggggaggcccgggccagcaccgggagctcagattcttcccctggtggccatgggatccaggtcaatgtcacctgcatcgtgaacgtctgtagcagctctgaccacagctcacagtgctcctcccaagccagctccacaatgggagacacagattccagcccctcggagtccccgaaggacgagcaggtccccttctccaaggaggaatgtgcctttcggtcacagctggagacgccagagaccctgctggggagcaccgaagagaagcccctgccccttggagtgcctgatgctgggatgaagcccagt

SEQ ID NO: 188 - protein, 461 aa, Synthetic construct
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGIQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 189 - DNA, 1383 bp, Synthetic construct
  source 1..1383: mol_type = other DNA
atggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcacttgcgacttcgctcttccagttggactgattgtgggtgtgacagccttgggtctactaataataggagtggtgaactgtgtcatcatgacccaggtgaaaaagaagcccttgtgcctgcagagagaagccaaggtgcctcacttgcctgccgataaggcccggggtacacagggccccgagcagcagcacctgctgatcacagcgccgagctccagcagcagctccctggagagctcggccagtgcgttggacagaagggcgcccactcggaaccagccacaggcaccaggcgtggaggccagtggggccggggaggcccgggccagcaccgggagctcagattcttcccctggtggccatgggacccaggtcaatgtcacctgcatcgtgaacgtctgtagcagctctgaccacagctcacagtgctcctcccaagccagctccacaatgggagacacagattccagcccctcggagtccccgaaggacgagcaggtccccttctccaaggaggaatgtgcctttcggtcacagctggagacgccagagaccctgctggggagcaccgaagagaagcccctgccccttggagtgcctgatgctgggatgaagcccagt

SEQ ID NO: 190 - protein, 461 aa, Synthetic construct
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTCDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 191 - DNA, 1383 bp, Homo sapiens
  source 1..1383: mol_type = genomic DNA
atggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacttcgctcttccagttggactgattgtgggtgtgacagccttgggtctactaataataggagtggtgaactgtgtcatcatgacccaggtgaaaaagaagcccttgtgcctgcagagagaagccaaggtgcctcacttgcctgccgataaggcccggggtacacagggccccgagcagcagcacctgctgatcacagcgccgagctccagcagcagctccctggagagctcggccagtgcgttggacagaagggcgcccactcggaaccagccacaggcaccaggcgtggaggccagtggggccggggaggcccgggccagcaccgggagctcagattcttcccctggtggccatgggacccaggtcaatgtcacctgcatcgtgaacgtctgtagcagctctgaccacagctcacagtgctcctcccaagccagctccacaatgggagacacagattccagcccctcggagtccccgaaggacgagcaggtccccttctccaaggaggaatgtgcctttcggtcacagctggagacgccagagaccctgctggggagcaccgaagagaagcccctgccccttggagtgcctgatgctgggatgaagcccagt

SEQ ID NO: 192 - protein, 461 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 193 - DNA, 1179 bp, Synthetic construct
  source 1..1179: mol_type = other DNA
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccatcatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcctgtttgtgcctgtcctgggagagaccggcgcacagaggaagagaatctccgtaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagac

SEQ ID NO: 194 - protein, 393 aa, Synthetic construct
MEEPQSDPSVEPPLSQETFSDLWKLLPENNVLSPLPSQAMDDLMLSPDDIEQWFTEDPGPDEAPRMPEAAPRVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVPVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 195 - DNA, 1179 bp, Homo sapiens
  source 1..1179: mol_type = genomic DNA
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccatcatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgtaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagac

SEQ ID NO: 196 - protein, 393 aa, Homo sapiens [sequence omitted]

SEQ ID NO: 197 - DNA, 2370 bp, Synthetic construct
  source 1..2370: mol_type = other DNA
atgattgtgctaccactgtactccagactggacaaaagattcctgtgccttaagaacattagaaccttcctgtccacctgctgtgagaagttcggcctcaagcggagcgagctcttcgaagcctttgacctcttcgatgtgcaggattttggcaaggtcatctacaccctgtctgctctgtcctggaccccgatcgcccagaacagggggatcatgcccttccccaccgaggaggagagtgtaggtgatgaagacatctacagtggcctgtccgaccagatcgacgacacggtggaggaggatgaggacctgtatgactgcgtggagaatgaggaggcggaaggcgacgagatctatgaggacctcatgcgctcggagcccgtgtccatgccgcccaagatgacagagtatgacaagcgctgctgctgcctgcgggagatccagcagacggaggagaagtacactgacacgctgggctccatccagcagcatttcttgaagcccctgcaacggttcctgaaacctcaagacattgagatcatctttatcaacattgaggacctgcttcgtgttcatactcacttcctaaaggagatgaaggaagccctgggcacccctggcgcagccaatctctaccaggtcttcatcaaatacaaggagaggttcctcgtctatggccgctactgcagccaggtggagtcagccagcaaacacctggaccgtgtggccgcagcccgggaggacgtgcagatgaagctggaggaatgttctcagagagccaacaacgggaggttcaccctgcgggacctgctgatggtgcctatgcagcgagttctcaaatatcacctccttctccaggagctggtgaaacacacgcaggaggcgatggagaaggagaacctgcggctggccctggatgccatgagggacctggctcagtgcgtgaacgaggtcaagcgagacaacgagacactgcgacagatcaccaatttccagctgtccattgagaacctggaccagtctctggctcactatggccggcccaagatcgacggggaactcaagatcacctcggtggaacggcgctccaagatggacaggtatgccttcctgctcgacaaagctctactcatctgtaagcgcaggggagactcctatgacctcaaggactttgtaaacctgcacagcttccaggttcgggatgactcttcaggagaccgagacaacaagaagtggagccacatgttcctcctgatcgaggaccaaggtgcccagggctatgagctgttcttcaagacaagagaattgaagaagaagtggatggagcagtttgagatggccatctccaacatctatccggagaatgccaccgccaacgggcatgacttccagatgttctcctttgaggagaccacatcctgcaaggcctgtcagatgctgcttagaggtaccttctatcagggctaccgctgccatcggtgccgggcatctgcacacaaggagtgtctggggagggtccctccatgtggccgacatgggcaagatttcccaggaactatgaagaaggacaaactacatcgcagggctcaggacaaaaagaggaatgagctgggtctgcccaagatggaggtgtttcaggaatactacgggcttcctccaccccctggagccattggaccctttctacggctcaaccctggagacattgtggagctcacgaaggctgaggctgaacagaactggtgggagggcagaaatacatctactaatgaaattggctggtttccttgtaacagggtgaagccctatgtccatggccctcctcaggacctgtctgttcatctctggtacgcaggccccatggagcgggcaggggcagagagcatcctggccaaccgctcggacgggactttcttggtgcggcagagggtgaaggatgcagcagaatttgccatcagcattaaatataacgtcgaggtcaagcacattaaaatcatgacagcagaaggactgtaccggatcacagagaaaaaggctttccgggggcttacggagctggtggagttttaccagcagaactctctaaaggattgcttcaagtctctggacaccaccttgcagttccccttcaaggagcctgaaaagagaaccatcagcaggccagcagtgggaagcacaaagtattttggcacagccaaagcccgctatgacttctgcgcccgacaccgatcagagctgtcgctcaaggagggtgacatcatcaagatccttaacaagaagggacagcaaggctggtggcgaggggagatctatggccgggttggctggttccctgccaactacgtggaggaagattattctgaatactgc

SEQ ID NO: 198 - protein, 790 aa, Synthetic construct
MIVLPLYSRLDKRFLCLKNIRTFLSTCCEKFGLKRSELFEAFDLFDVQDFGKVIYTLSALSWTPIAQNRGIMPFPTEEESVGDEDIYSGLSDQIDDTVEEDEDLYDCVENEEAEGDEIYEDLMRSEPVSMPPKMTEYDKRCCCLREIQQTEEKYTDTLGSIQQHFLKPLQRFLKPQDIEIIFINIEDLLRVHTHFLKEMKEALGTPGAANLYQVFIKYKERFLVYGRYCSQVESASKHLDRVAAAREDVQMKLEECSQRANNGRFTLRDLLMVPMQRVLKYHLLLQELVKHTQEAMEKENLRLALDAMRDLAQCVNEVKRDNETLRQITNFQLSIENLDQSLAHYGRPKIDGELKITSVERRSKMDRYAFLLDKALLICKRRGDSYDLKDFVNLHSFQVRDDSSGDRDNKKWSHMFLLIEDQGAQGYELFFKTRELKKKWMEQFEMAISNIYPENATANGHDFQMFSFEETTSCKACQMLLRGTFYQGYRCHRCRASAHKECLGRVPPCGRHGQDFPGTMKKDKLHRRAQDKKRNELGLPKMEVFQEYYGLPPPPGAIGPFLRLNPGDIVELTKAEAEQNWWEGRNTSTNEIGWFPCNRVKPYVHGPPQDLSVHLWYAGPMERAGAESILANRSDGTFLVRQRVKDAAEFAISIKYNVEVKHIKIMTAEGLYRITEKKAFRGLTELVEFYQQNSLKDCFKSLDTTLQFPFKEPEKRTISRPAVGSTKYFGTAKARYDFCARHRSELSLKEGDIIKILNKKGQQGWWRGEIYGRVGWFPANYVEEDYSEYC

SEQ ID NO: 199 - DNA, 2370 bp, Synthetic construct
  source 1..2370: mol_type = other DNA
atgattgtgctaccactgtactccagactggacaaaagattcctgtgccttaagaacattagaaccttcctgtccacctgctgtgagaagttcggcctcaagcggagcgagctcttcgaagcctttgacctcttcgatgtgcaggattttggcaaggtcatctacaccctgtctgctctgtcctggaccccgatcgcccagaacagggggatcatgcccttccccaccgaggaggagagtgtaggtgatgaagacatctacagtggcctgtccgaccagatcgacgacacggtggaggaggatgaggacctgtatgactgcgtggagaatgaggaggcggaaggcgacgagatctatgaggacctcatgcgctcggagcccgtgtccatgccgcccaagatgacagagtatgacaagcgctgctgctgcctgcgggagatccagcagacggaggagaagtacactgacacgctgggctccatccagcagcatttcttgaagcccctgcaacggttcctgaaacctcaagacattgagatcatctttatcaacattgaggacctgcttcgtgttcatactcacttcctaaaggagatgaaggaagccctgggcacccctggcgcagccaatctctaccaggtcttcatcaaatacaaggagaggttcctcgtctatggccgctactgcagccaggtggagtcagccagcaaacacctggaccgtgtggccgcagcccgggaggacgtgcagatgaagctggaggaatgttctcagagagccaacaacgggaggttcaccctgcgggacctgctgatggtgcctatgcagcgagttctcaaatatcacctccttctccaggagctggtgaaacacacgcaggaggcgatggagaaggagaacctgcggctggccctggatgccatgagggacctggctcagtgcgtgaacgaggtcaagcgagacaacgagacactgcgacagatcaccaatttccagctgtccattgagaacctggaccagtctctggctcactatggccggcccaagatcgacggggaactcaagatcacctcggtggaacggcgctccaagatggacaggtatgccttcctgctcgacaaagctctactcatctgtaagcgcaggggagactcctatgacctcaaggactttgtaaacctgcacagcttccaggttcgggatgactcttcaggagaccgagacaacaagaagtggagccacatgttcctcctgatcgaggaccaaggtgcccagggctatgagctgttcttcaagacaagagaattgaagaagaagtggatggagcagtttgagatggccatctccaacatctatccggagaatgccaccgccaacgggcatgacttccagatgttctcctttgaggagaccacatcctgcaaggcctgtcagatgctgcttagaggtaccttctatcagggctaccgctgccatcggtgccgggcatctgcacacaaggactgtctggggagggtccctccatgtggccgacatgggcaagatttcccaggaactatgaagaaggacaaactacatcgcagggctcaggacaaaaagaggaatgagctgggtctgcccaagatggaggtgtttcaggaatactacgggcttcctccaccccctggagccattggaccctttctacggctcaaccctggagacattgtggagctcacgaaggctgaggctgaacagaactggtgggagggcagaaatacatctactaatgaaattggctggtttccttgtaacagggtgaagccctatgtccatggccctcctcaggacctgtctgttcatctctggtacgcaggccccatggagcgggcaggggcagagagcatcctggccaaccgctcggacgggactttcttggtgcggcagagggtgaaggatgcagcagaatttgccatcagcattaaatataacgtcgaggtcaagcacattaaaatcatgacagcagaaggactgtaccggatcacagagaaaaaggctttccgggggcttacggagctggtggagttttaccagcagaactctctaaaggattgcttcaagtctctggacaccaccttgcagttccccttcaaggagcctgaaaagagaaccatcagcaggccagcagtgggaagcacaaagtattttggcacagccaaagcccgctatgacttctgcgcccgagaccgatcagagctgtcgctcaaggagggtgacatcatcaagatccttaacaagaagggacagcaaggctggtggcgaggggagatctatggccgggttggctggttccctgccaactacgtggaggaagattattctgaatactgc

SEQ ID NO: 200 - protein, 790 aa, Synthetic construct
MIVLPLYSRLDKRFLCLKNIRTFLSTCCEKFGLKRSELFEAFDLFDVQDFGKVIYTLSALSWTPIAQNRGIMPFPTEEESVGDEDIYSGLSDQIDDTVEEDEDLYDCVENEEAEGDEIYEDLMRSEPVSMPPKMTEYDKRCCCLREIQQTEEKYTDTLGSIQQHFLKPLQRFLKPQDIEIIFINIEDLLRVHTHFLKEMKEALGTPGAANLYQVFIKYKERFLVYGRYCSQVESASKHLDRVAAAREDVQMKLEECSQRANNGRFTLRDLLMVPMQRVLKYHLLLQELVKHTQEAMEKENLRLALDAMRDLAQCVNEVKRDNETLRQITNFQLSIENLDQSLAHYGRPKIDGELKITSVERRSKMDRYAFLLDKALLICKRRGDSYDLKDFVNLHSFQVRDDSSGDRDNKKWSHMFLLIEDQGAQGYELFFKTRELKKKWMEQFEMAISNIYPENATANGHDFQMFSFEETTSCKACQMLLRGTFYQGYRCHRCRASAHKDCLGRVPPCGRHGQDFPGTMKKDKLHRRAQDKKRNELGLPKMEVFQEYYGLPPPPGAIGPFLRLNPGDIVELTKAEAEQNWWEGRNTSTNEIGWFPCNRVKPYVHGPPQDLSVHLWYAGPMERAGAESILANRSDGTFLVRQRVKDAAEFAISIKYNVEVKHIKIMTAEGLYRITEKKAFRGLTELVEFYQQNSLKDCFKSLDTTLQFPFKEPEKRTISRPAVGSTKYFGTAKARYDFCARDRSELSLKEGDIIKILNKKGQQGWWRGEIYGRVGWFPANYVEEDYSEYC

SEQ ID NO: 201 - DNA, 2370 bp, Synthetic construct
  source 1..2370: mol_type = other DNA
atgattgtgctaccactgtactccagactggacaaaagattcctgtgccttaagaacattagaaccttcctgtccacctgctgtgagaagttcggcctcaagcggagcgagctcttcgaagcctttgacctcttcgatgtgcaggattttggcaaggtcatctacaccctgtctgctctgtcctggaccccgatcgcccagaacagggggatcatgcccttccccaccgaggaggagagtgtaggtgatgaagacatctacagtggcctgtccgaccagatcgacgacacggtggaggaggatgaggacctgtatgactgcgtggagaatgaggaggcggaaggcgacgagatctatgaggacctcatgcgctcggagcccgtgtccatgccgcccaagatgacagagtatgacaagcgctgctgctgcctgcgggagatccagcagacggaggagaagtacactgacacgctgggctccatccagcagcatttcttgaagcccctgcaacggttcctgaaacctcaagacattgagatcatctttatcaacattgaggacctgcttcgtgttcatactcacttcctaaaggagatgaaggaagccctgggcacccctggcgcagccaatctctaccaggtcttcatcaaatacaaggagaggttcctcgtctatggccgctactgcagccaggtggagtcagccagcaaacacctggaccgtgtggccgcagcccgggaggacgtgcagatgaagctggaggaatgttctcagagagccaacaacgggaggttcaccctgcgggacctgctgatggtgcctatgcagcgagttctcaaatatcacctccttctccaggagctggtgaaacacacgcaggaggcgatggagaaggagaacctgcggctggccctggatgccatgagggacctggctcagtgcgtgaacgaggtcaagcgagacaacgagacactgcgacagatcaccaatttccagctgtccattgagaacctggaccagtctctggctcactatggccggcccaagatcgacggggaactcaagatcacctcggtggaacggcgctccaagatggacaggtatgccttcctgctcgacaaagctctactcatctgtaagcgcaggggagactcctatgacctcaaggactttgtaaacctgcacagcttccaggttcgggatgactcttcaggagaccgagacaacaagaagtggagccacatgttcctcctgatcgaggaccaaggtgcccagggctatgagctgttcttcaagacaagagaattgaagaagaagtggatggagcagtttgagatggccatctccaacatctatccggagaatgccaccgccaacgggcatgacttccagatgttctcctttgaggagaccacatcctgcaaggcctgtcagatgctgcttagaggtaccttctatcagggctaccgctgccatcggtgccgggcatctgcacacaaggagtgtctggggagggtccctccatgtggccgacatgggcaagatttcccaggaactatgaagaaggacaaactacatcgcagggctcaggacaaaaagaggaatgagctgggtctgcccaagatggaggtgtttcaggaatactacgggcttcctccaccccctggagccattggaccctttctacggctcaaccctggagacattgtggagctcacgaaggctgaggctgaacagaactggtgggagggcagaaatacatctactaatgaaattggctggtttccttgtaacagggtgaagccctatgtccatggccctcctcaggacctgtctgttcatctctggtacgcaggccccatggagcgggcaggggcagagagcatcctggccaaccgctcggacgggactttcttggtgcggcagagggtgaaggatgcagcagaatttgccatcagcattaaatataacgtcgaggtcaagcacattaaaatcatgacagcagaaggactgtaccggatcacagagaaaaaggctttccgggggcttacggagctggtggagttttaccagcagaactctctaaaggattgcttcaagtctctggacaccaccttgcagttccccttcaaggagcctgaaaagagaaccatcagcaggccagcagtgggaagcacaaagtattttggcacagccaaagcccgctatgacttctgcgcccgagaccaatcagagctgtcgctcaaggagggtgacatcatcaagatccttaacaagaagggacagcaaggctggtggcgaggggagatctatggccgggttggctggttccctgccaactacgtggaggaagattattctgaatactgc

SEQ ID NO: 202 - protein, 790 aa, Synthetic construct
MIVLPLYSRLDKRFLCLKNIRTFLSTCCEKFGLKRSELFEAFDLFDVQDFGKVIYTLSALSWTPIAQNRGIMPFPTEEESVGDEDIYSGLSDQIDDTVEEDEDLYDCVENEEAEGDEIYEDLMRSEPVSMPPKMTEYDKRCCCLREIQQTEEKYTDTLGSIQQHFLKPLQRFLKPQDIEIIFINIEDLLRVHTHFLKEMKEALGTPGAANLYQVFIKYKERFLVYGRYCSQVESASKHLDRVAAAREDVQMKLEECSQRANNGRFTLRDLLMVPMQRVLKYHLLLQELVKHTQEAMEKENLRLALDAMRDLAQCVNEVKRDNETLRQITNFQLSIENLDQSLAHYGRPKIDGELKITSVERRSKMDRYAFLLDKALLICKRRGDSYDLKDFVNLHSFQVRDDSSGDRDNKKWSHMFLLIEDQGAQGYELFFKTRELKKKWMEQFEMAISNIYPENATANGHDFQMFSFEETTSCKACQMLLRGTFYQGYRCHRCRASAHKECLGRVPPCGRHGQDFPGTMKKDKLHRRAQDKKRNELGLPKMEVFQEYYGLPPPPGAIGPFLRLNPGDIVELTKAEAEQNWWEGRNTSTNEIGWFPCNRVKPYVHGPPQDLSVHLWYAGPMERAGAESILANRSDGTFLVRQRVKDAAEFAISIKYNVEVKHIKIMTAEGLYRITEKKAFRGLTELVEFYQQNSLKDCFKSLDTTLQFPFKEPEKRTISRPAVGSTKYFGTAKARYDFCARDQSELSLKEGDIIKILNKKGQQGWWRGEIYGRVGWFPANYVEEDYSEYC

SEQ ID NO: 203 - DNA, 2370 bp, Homo sapiens
  source 1..2370: mol_type = genomic DNA
atgattgtgctaccactgtactccagactggacaaaagattcctgtgccttaagaacattagaaccttcctgtccacctgctgtgagaagttcggcctcaagcggagcgagctcttcgaagcctttgacctcttcgatgtgcaggattttggcaaggtcatctacaccctgtctgctctgtcctggaccccgatcgcccagaacagggggatcatgcccttccccaccgaggaggagagtgtaggtgatgaagacatctacagtggcctgtccgaccagatcgacgacacggtggaggaggatgaggacctgtatgactgcgtggagaatgaggaggcggaaggcgacgagatctatgaggacctcatgcgctcggagcccgtgtccatgccgcccaagatgacagagtatgacaagcgctgctgctgcctgcgggagatccagcagacggaggagaagtacactgacacgctgggctccatccagcagcatttcttgaagcccctgcaacggttcctgaaacctcaagacattgagatcatctttatcaacattgaggacctgcttcgtgttcatactcacttcctaaaggagatgaaggaagccctgggcacccctggcgcagccaatctctaccaggtcttcatcaaatacaaggagaggttcctcgtctatggccgctactgcagccaggtggagtcagccagcaaacacctggaccgtgtggccgcagcccgggaggacgtgcagatgaagctggaggaatgttctcagagagccaacaacgggaggttcaccctgcgggacctgctgatggtgcctatgcagcgagttctcaaatatcacctccttctccaggagctggtgaaacacacgcaggaggcgatggagaaggagaacctgcggctggccctggatgccatgagggacctggctcagtgcgtgaacgaggtcaagcgagacaacgagacactgcgacagatcaccaatttccagctgtccattgagaacctggaccagtctctggctcactatggccggcccaagatcgacggggaactcaagatcacctcggtggaacggcgctccaagatggacaggtatgccttcctgctcgacaaagctctactcatctgtaagcgcaggggagactcctatgacctcaaggactttgtaaacctgcacagcttccaggttcgggatgactcttcaggagaccgagacaacaagaagtggagccacatgttcctcctgatcgaggaccaaggtgcccagggctatgagctgttcttcaagacaagagaattgaagaagaagtggatggagcagtttgagatggccatctccaacatctatccggagaatgccaccgccaacgggcatgacttccagatgttctcctttgaggagaccacatcctgcaaggcctgtcagatgctgcttagaggtaccttctatcagggctaccgctgccatcggtgccgggcatctgcacacaaggagtgtctggggagggtccctccatgtggccgacatgggcaagatttcccaggaactatgaagaaggacaaactacatcgcagggctcaggacaaaaagaggaatgagctgggtctgcccaagatggaggtgtttcaggaatactacgggcttcctccaccccctggagccattggaccctttctacggctcaaccctggagacattgtggagctcacgaaggctgaggctgaacagaactggtgggagggcagaaatacatctactaatgaaattggctggtttccttgtaacagggtgaagccctatgtccatggccctcctcaggacctgtctgttcatctctggtacgcaggccccatggagcgggcaggggcagagagcatcctggccaaccgctcggacgggactttcttggtgcggcagagggtgaaggatgcagcagaatttgccatcagcattaaatataacgtcgaggtcaagcacattaaaatcatgacagcagaaggactgtaccggatcacagagaaaaaggctttccgggggcttacggagctggtggagttttaccagcagaactctctaaaggattgcttcaagtctctggacaccaccttgcagttccccttcaaggagcctgaaaagagaaccatcagcaggccagcagtgggaagcacaaagtattttggcacagccaaagcccgctatgacttctgcgcccgagaccgatcagagctgtcgctcaaggagggtgacatcatcaagatccttaacaagaagggacagcaaggctggtggcgaggggagatctatggccgggttggctggttccctgccaactacgtggaggaagattattctgaatactgc

SEQ ID NO: 204 - protein, 790 aa, Homo sapiens
MIVLPLYSRLDKRFLCLKNIRTFLSTCCEKFGLKRSELFEAFDLFDVQDFGKVIYTLSALSWTPIAQNRGIMPFPTEEESVGDEDIYSGLSDQIDDTVEEDEDLYDCVENEEAEGDEIYEDLMRSEPVSMPPKMTEYDKRCCCLREIQQTEEKYTDTLGSIQQHFLKPLQRFLKPQDIEIIFINIEDLLRVHTHFLKEMKEALGTPGAANLYQVFIKYKERFLVYGRYCSQVESASKHLDRVAAAREDVQMKLEECSQRANNGRFTLRDLLMVPMQRVLKYHLLLQELVKHTQEAMEKENLRLALDAMRDLAQCVNEVKRDNETLRQITNFQLSIENLDQSLAHYGRPKIDGELKITSVERRSKMDRYAFLLDKALLICKRRGDSYDLKDFVNLHSFQVRDDSSGDRDNKKWSHMFLLIEDQGAQGYELFFKTRELKKKWMEQFEMAISNIYPENATANGHDFQMFSFEETTSCKACQMLLRGTFYQGYRCHRCRASAHKECLGRVPPCGRHGQDFPGTMKKDKLHRRAQDKKRNELGLPKMEVFQEYYGLPPPPGAIGPFLRLNPGDIVELTKAEAEQNWWEGRNTSTNEIGWFPCNRVKPYVHGPPQDLSVHLWYAGPMERAGAESILANRSDGTFLVRQRVKDAAEFAISIKYNVEVKHIKIMTAEGLYRITEKKAFRGLTELVEFYQQNSLKDCFKSLDTTLQFPFKEPEKRTISRPAVGSTKYFGTAKARYDFCARDRSELSLKEGDIIKILNKKGQQGWWRGEIYGRVGWFPANYVEEDYSEYC

SEQ ID NO: 205 - DNA, 2175 bp, Homo sapiens
  source 1..2175: mol_type = genomic DNA
atggatgactacatggagacgctgaaggatgaagaggacgccttgtgggagaatgtggagtgtaaccggcacatgctcagccgctatatcaaccctgccaagctcacgccctacctgcgtcagtgtaaggtcattgatgagcaggatgaagatgaagtgcttaatgcccctatgctgccatccaagatcaaccgagcaggccggctgttggacattctacataccaaggggcaaaggggctatgtggtcttcttggagagcctagaattttattacccagaactgtacaaactggtgactgggaaagagcccactcggagattctccaccattgtggtggaggaaggccacgagggcctcacgcacttcctgatgaacgaggtcatcaagctgcagcagcagatgaaggccaaggacctgcaacgctgcgagctgctggccaggttgcggcagctggaggatgagaagaagcagatgacgctgacgcgcgtggagctgctaaccttccaggagcggtactacaagatgaaggaagagcgggacagctacaatgacgagctggtcaaggtgaaggacgacaactacaacttagccatgcgctacgcacagctcagtgaggagaagaacatggcggtcatgaggagccgagacctccaactcgagatcgatcagctaaagcaccggttgaataagatggaggaggaatgtaagctggagagaaatcagtctctaaaactgaagaatgacattgaaaatcggcccaagaaggagcaggttctggaactggagcgggagaatgaaatgctgaagaccaaaaaccaggagctgcagtccatcatccaggccgggaagcgcagcctgccagactcagacaaggccatcctggacatcttggaacacgaccgcaaggaggccctggaggacaggcaggagctggtcaacaggatctacaacctgcaggaggaggcccgccaggcagaggagctgcgagacaagtacctggaggagaaggaggacctggagctcaagtgctcgaccctgggaaaggactgtgaaatgtacaagcaccgcatgaacacggtcatgctgcagctggaggaggtggagcgggagcgggaccaggccttccactcccgagatgaagctcagacacagtactcgcagtgcttaatcgaaaaggacaagtacaggaagcagatccgcgagctggaggagaagaacgatgagatgaggatcgagatggtgcggcgggaggcctgcatcgtcaacctggagagcaagctgcggcgcctctccaaggacagcaacaacctggaccagagtctgcccaggaacctgccagtaaccatcatctctcaggactttggggatgccagccccaggaccaatggtcaagaagctgacgattcttccacctcggaggagtcacctgaagacagcaagtacttcctgccctaccatccgccccagcgcaggatgaacctgaagggcatccagctgcagagagccaaatcccccatcagcctgaagcgaacatcagattttcaagccaaggggcacgaggaagaaggcacggatgccagccctagctcctgcggatctctgcccatcaccaactccttcaccaagatgcagcccccccggagccgcagcagcatcatgtcaatcaccgccgagcccccgggaaacgactccatcgtcagacgctacaaggaggacgcgccccatcgcagatggctcaatcacaaaggagtgagacagaaacgcctgaatgtctggctgggaattaagaatgaggatgctgctgagaactattttatcaatgaggaagatgaaaacctgccccattatgatgagaaaacctggtttgttgaggatatcaatcgagtacaagcagaggacttgctttatgggaaacctgatggtgcattcttaattcgtgagagtagcaagaaaggatgctatgcttgctctgtggtggccgatggggaagtgaagcactgtgtgatctacagcactgctcggggctatggctttgcagagccctacaacctgtacagctctctgaaggagctagtgctccattaccagcagacatccttggttcagcacaacgactccctcaacgtcaggcttgcctaccctgttcatgcacagatgccctcgctttgcaga

SEQ ID NO: 206 - protein, 725 aa, Homo sapiens
MDDYMETLKDEEDALWENVECNRHMLSRYINPAKLTPYLRQCKVIDEQDEDEVLNAPMLPSKINRAGRLLDILHTKGQRGYVVFLESLEFYYPELYKLVTGKEPTRRFSTIVVEEGHEGLTHFLMNEVIKLQQQMKAKDLQRCELLARLRQLEDEKKQMTLTRVELLTFQERYYKMKEERDSYNDELVKVKDDNYNLAMRYAQLSEEKNMAVMRSRDLQLEIDQLKHRLNKMEEECKLERNQSLKLKNDIENRPKKEQVLELERENEMLKTKNQELQSIIQAGKRSLPDSDKAILDILEHDRKEALEDRQELVNRIYNLQEEARQAEELRDKYLEEKEDLELKCSTLGKDCEMYKHRMNTVMLQLEEVERERDQAFHSRDEAQTQYSQCLIEKDKYRKQIRELEEKNDEMRIEMVRREACIVNLESKLRRLSKDSNNLDQSLPRNLPVTIISQDFGDASPRTNGQEADDSSTSEESPEDSKYFLPYHPPQRRMNLKGIQLQRAKSPISLKRTSDFQAKGHEEEGTDASPSSCGSLPITNSFTKMQPPRSRSSIMSITAEPPGNDSIVRRYKEDAPHRRWLNHKGVRQKRLNVWLGIKNEDAAENYFINEEDENLPHYDEKTWFVEDINRVQAEDLLYGKPDGAFLIRESSKKGCYACSVVADGEVKHCVIYSTARGYGFAEPYNLYSSLKELVLHYQQTSLVQHNDSLNVRLAYPVHAQMPSLCR

SEQ ID NO: 207 - DNA, 2040 bp, Homo sapiens
  source 1..2040: mol_type = genomic DNA
atggaagattcgatggacatggacatgagccccctgaggccccagaactatcttttcggttgtgaactaaaggccgacaaagattatcactttaaggtggataatgatgaaaatgagcaccagttatctttaagaacggtcagtttaggggctggtgcaaaggatgagttgcacattgttgaagcagaggcaatgaattacgaaggcagtccaattaaagtaacactggcaactttgaaaatgtctgtacagccaacggtttcccttgggggctttgaaataacaccaccagtggtcttaaggttgaagtgtggttcagggccagtgcatattagtggacagcacttagtagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccc

SEQ ID NO: 208 - protein, 680 aa, Homo sapiens
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVVYRRKHQELQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEVSVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 209 - DNA, 1458 bp, Homo sapiens
  source 1..1458: mol_type = genomic DNA
atgaacaactttatcctcctggaagaacagctcatcaagaaatcccaacaaaagagaagaacttctccctcgaactttaaagtccgcttctttgtgttaaccaaagccagcctggcatactttgaagatcgtcatgggaagaagcgcacgctgaaggggtccattgagctctcccgaatcaaatgtgttgagattgtgaaaagtgacatcagcatcccatgccactataaatacccgtttcaggtggtgcatgacaactacctcctatatgtgtttgctccagatcgtgagagccggcagcgctgggtgctggcccttaaagaagaaacgaggaataataacagtttggtgcctaaatatcatcctaatttctggatggatgggaagtggaggtgctgttctcagctggagaagcttgcaacaggctgtgcccaatatgatccaaccaagaatgcttcaaagaagcctcttcctcctactcctgaagacaacaggtcctcccctgcccaagggaaccggcaagagagtactgtgtcattcaatccgtatgagccagaacttgcaccctgggctgcagacaaaggcccccagagagaagccctacccatggacacagaggtgtacgagagcccctacgcggaccctgaggagatcaggcccaaggaggtttacctggaccgaaagctgctgacgctggaagacaaagaactgggctctggtaattttggaactgtgaaaaagggctactaccaaatgaaaaaagttgtgaaaaccgtggctgtgaaaatactgaaaaacgaggccaatgaccccgctcttaaagatgagttattagcagaagcaaatgtcatgcagcagctggacaacccgtacatcgtgcgcatgatcgggatatgcgaggccgagtcctggatgctagttatggagatggcagaacttggtcccctcaataagtatttgcagcagaacagacatgtcaaggataagaacatcatagaactggttcatcaggtttccatgggcatgaagtacttggaggagagcaattttgtgcacagagatctggctgcaagaaatgtgttgctagttacccaacattatgccaagatcagtgatttcggactctccaaagcactgcgtgctgatgaaaactactacaaggcccagacccatggaaagtggcctgtcaagtggtacgctccggaatgcatcaactactacaagttctccagcaaaagcgatgtctggagctttggagtgttgatgtgggaagcattctcctatgggcagaagccatatcgagggatgaaaggaagtgaagtcaccgctatgttagagaaaggagagcggatggggtgccctgcagggtgtccaagagagatgtacgatctcatgaatctgtgctggacatacgatgtggaaaacaggcccggattcgcagcagtggaactgcggctg

SEQ ID NO: 210 - protein, 486 aa, Homo sapiens
MNNFILLEEQLIKKSQQKRRTSPSNFKVRFFVLTKASLAYFEDRHGKKRTLKGSIELSRIKCVEIVKSDISIPCHYKYPFQVVHDNYLLYVFAPDRESRQRWVLALKEETRNNNSLVPKYHPNFWMDGKWRCCSQLEKLATGCAQYDPTKNASKKPLPPTPEDNRSSPAQGNRQESTVSFNPYEPELAPWAADKGPQREALPMDTEVYESPYADPEEIRPKEVYLDRKLLTLEDKELGSGNFGTVKKGYYQMKKVVKTVAVKILKNEANDPALKDELLAEANVMQQLDNPYIVRMIGICEAESWMLVMEMAELGPLNKYLQQNRHVKDKNIIELVHQVSMGMKYLEESNFVHRDLAARNVLLVTQHYAKISDFGLSKALRADENYYKAQTHGKWPVKWYAPECINYYKFSSKSDVWSFGVLMWEAFSYGQKPYRGMKGSEVTAMLEKGERMGCPAGCPREMYDLMNLCWTYDVENRPGFAAVELRL

SEQ ID NO: 211 - DNA, 1998 bp, Homo sapiens
  source 1..1998: mol_type = genomic DNA
atgaacaactttatcctcctggaagaacagctcatcaagaaatcccaacaaaagagaagaacttctccctcgaactttaaagtccgcttctttgtgttaaccaaagccagcctggcatactttgaagatcgtcatgggaagaagcgcacgctgaaggggtccattgagctctcccgaatcaaatgtgttgagattgtgaaaagtgacatcagcatcccatgccactataaatacccgtttcaggtggtgcatgacaactacctcctatatgtgtttgctccagatcgtgagagccggcagcgctgggtgctggcccttaaagaagaaacgaggaataataacagtttggtgcctaaatatcatcctaatttctggatggatgggaagtggaggtgctgttctcagctggagaagcttgcaacaggctgtgcccaatatgatccaaccaagaatgcttcaaagaagcctcttcctcctactcctgaagacaacaggcgaccactttgggaacctgaagaaactgtggtcattgccttatatgactaccaaaccaatgatcctcaggaactcgcactgcggcgcaacgaagagtactgcctgctggacagttctgagattcactggtggagagtccaggacaggaatgggcatgaaggatatgtaccaagcagttatctggtggaaaaatctccaaataatctggaaacctatgagtggtacaataagagtatcagccgagacaaagctgaaaaacttcttttggacacagaaagaaaggagaggctatccaaatttgaatctattcgtcattcaattgctggaataattaggtctccaaaatctgcactgggctcttcagcactttctgatatgatctccatcagtgagaagcctttggcagaacaggactggtaccatggtgcaattcccagaatagaagctcaagaactgttaaaaaaacaaggagactttttggtgcgagagagtcatgggaaacctggtgaatatgtcctttctgtatattctgatggacagaggagacattttatcatacaatatgttgataacatgtatcgattcgagggcactgggttttcaaacattcctcaacttatagatcatcactatacaacaaaacaggtcatcactaagaaatcaggtgtagttctgctgaatcctattcctaaggacaagaaatggattctcagtcatgaagatgtcatattgggagaattactgggcaagggaaattttggtgaagtatataagggcacattaaaggataaaacttctgttgctgttaaaacatgtaaagaagatcttcctcaggaattgaaaataaaatttttacaagaagccaaaattctcaagcaatatgatcatcccaatattgtcaaacttataggagtttgcacacaaagacagcctgtctacatcattatggaactggtttcaggaggtgatttcctcacctttctgagaaggaagaaggatgaactaaaactcaaacagttagtgaaattttcattagacgctgctgctggtatgttgtatctcgagagtaaaaactgtatacacagggaccttgctgcaagaaactgcctggtaggtgaaaataatgttctgaaaatcagtgactttggaatgtctcgtcaagaggatggtggagtgtattcatcttctggcttaaagcagattcccattaaatggacagcaccggaagctcttaattatgggagatacagttcagagagtgacgtgtggagctttgtcatccttctctgggagaccttcagcttaggggtttgtccgtaccctggaatgacaaatcagcaagcaagagagcaagtagaaagaggataccggatgtcagctccccagcactgtccagaggatatttccaaaattatgatgaagtgttgggattataaacctgaaaatcgccctaagttcagtgaacttcagaaagagctcactatcataaagagaaaactcaca

SEQ ID NO: 212 - protein, 666 aa, Homo sapiens
MNNFILLEEQLIKKSQQKRRTSPSNFKVRFFVLTKASLAYFEDRHGKKRTLKGSIELSRIKCVEIVKSDISIPCHYKYPFQVVHDNYLLYVFAPDRESRQRWVLALKEETRNNNSLVPKYHPNFWMDGKWRCCSQLEKLATGCAQYDPTKNASKKPLPPTPEDNRRPLWEPEETVVIALYDYQTNDPQELALRRNEEYCLLDSSEIHWWRVQDRNGHEGYVPSSYLVEKSPNNLETYEWYNKSISRDKAEKLLLDTERKERLSKFESIRHSIAGIIRSPKSALGSSALSDMISISEKPLAEQDWYHGAIPRIEAQELLKKQGDFLVRESHGKPGEYVLSVYSDGQRRHFIIQYVDNMYRFEGTGFSNIPQLIDHHYTTKQVITKKSGVVLLNPIPKDKKWILSHEDVILGELLGKGNFGEVYKGTLKDKTSVAVKTCKEDLPQELKIKFLQEAKILKQYDHPNIVKLIGVCTQRQPVYIIMELVSGGDFLTFLRRKKDELKLKQLVKFSLDAAAGMLYLESKNCIHRDLAARNCLVGENNVLKISDFGMSRQEDGGVYSSSGLKQIPIKWTAPEALNYGRYSSESDVWSFVILLWETFSLGVCPYPGMTNQQAREQVERGYRMSAPQHCPEDISKIMMKCWDYKPENRPKFSELQKELTIIKRKLT

SEQ ID NO: 213 - DNA, 3597 bp, Homo sapiens
  source 1..3597: mol_type = genomic DNA
atgattgtgctaccactgtactccagactggacaaaagattcctgtgccttaagaacattagaaccttcctgtccacctgctgtgagaagttcggcctcaagcggagcgagctcttcgaagcctttgacctcttcgatgtgcaggattttggcaaggtcatctacaccctgtctgctctgtcctggaccccgatcgcccagaacagggggatcatgcccttccccaccgaggaggagagtgtaggtgatgaagacatctacagtggcctgtccgaccagatcgacgacacggtggaggaggatgaggacctgtatgactgcgtggagaatgaggaggcggaaggcgacgagatctatgaggacctcatgcgctcggagcccgtgtccatgccgcccaagatgacagagtatgacaagcgctgctgctgcctgcgggagatccagcagacggaggagaagtacactgacacgctgggctccatccagcagcatttcttgaagcccctgcaacggttcctgaaacctcaagacattgagatcatctttatcaacattgaggacctgcttcgtgttcatactcacttcctaaaggagatgaaggaagccctgggcacccctggcgcagccaatctctaccaggtcttcatcaaatacaaggagaggttcctcgtctatggccgctactgcagccaggtggagtcagccagcaaacacctggaccgtgtggccgcagcccgggaggacgtgcagatgaagctggaggaatgttctcagagagccaacaacgggaggttcaccctgcgggacctgctgatggtgcctatgcagcgagttctcaaatatcacctccttctccaggagctggtgaaacacacgcaggaggcgatggagaaggagaacctgcggctggccctggatgccatgagggacctggctcagtgcgtgaacgaggtcaagcgagacaacgagacactgcgacagatcaccaatttccagctgtccattgagaacctggaccagtctctggctcactatggccggcccaagatcgacggggaactcaagatcacctcggtggaacggcgctccaagatggacaggtatgccttcctgctcgacaaagctctactcatctgtaagcgcaggggagactcctatgacctcaaggactttgtaaacctgcacagcttccaggttcgggatgactcttcaggagaccgagacaacaagaagtggagccacatgttcctcctgatcgaggaccaaggtgcccagggctatgagctgttcttcaagacaagagaattgaagaagaagtggatggagcagtttgagatggccatctccaacatctatccggagaatgccaccgccaacgggcatgacttccagatgttctcctttgaggagaccacatcctgcaaggcctgtcagatgctgcttagaggtaccttctatcagggctaccgctgccatcggtgccgggcatctgcacacaaggagtgtctggggagggtccctccatgtggccgacatgggcaagatttcccaggaactatgaagaaggacaaactacatcgcagggctcaggacaaaaagaggaatgagctgggtctgcccaagatggaggtgtttcaggaatactacgggcttcctccaccccctggagccattggaccctttctacggctcaaccctggagacattgtggagctcacgaaggctgaggctgaacagaactggtgggagggcagaaatacatctactaatgaaattggctggtttccttgtaacagggtgaagccctatgtccatggccctcctcaggacctgtctgttcatctctggtacgcaggccccatggagcgggcaggggcagagagcatcctggccaaccgctcggacgggactttcttggtgcggcagagggtgaaggatgcagcagaatttgccatcagcattaaatataacgtcgaggtcaagcacattaaaatcatgacagcagaaggactgtaccggatcacagagaaaaaggctttccgggggcttacggagctggtggagttttaccagcagaactctctaaaggattgcttcaagtctctggacaccaccttgcagttccccttcaaggagcctgaaaagagaaccatcagcaggccagcaggtgttgggatggccaccaactgggggagcctcttgcaggataaacagcagctagaggagctggcacggcaggccgtggaccgggccctggctgagggagtattgctgaggacctcacaggagcccacttcctcggaggtggtgagctatgccccattcacgctcttcccctcactggtccccagtgccctgctggagcaagcctatgctgtgcagatggacttcaacctgctagtggatgctgtcagccagaacgctgccttcctggagcaaactctttccagcaccatcaaacaggatgactttaccgctcgtctctttgacatccacaagcaagtcctaaaagagggcattgcccagactgtgttcctgggcctgaatcgctcagactacatgttccagcgcagcgcagatggctccccagccctgaaacagatcgaaatcaacaccatctctgccagctttgggggcctggcctcccggaccccagctgtgcaccgacatgttctcagtgtcctgagtaagaccaaagaagctggcaagatcctctctaataatcccagcaagggactggccctgggaattgccaaagcctgggagctctacggctcacccaatgctctggtgctactgattgctcaagagaaggaaagaaacatatttgaccagcgtgccatagagaatgagctactggccaggaacatccatgtgatccgacgaacatttgaagatatctctgaaaaggggtctctggaccaagaccgaaggctgtttgtggatggccaggaaattgctgtggtttacttccgggatggctacatgcctcgtcagtacagtctacagaattgggaagcacgtctactgctggagaggtcacatgctgccaagtgcccagacattgccacccagctggctgggactaagaaggtgcagcaggagctaagcaggccgggcatgctggagatgttgctccctggccagcctgaggctgtggcccgcctccgcgccacctttgctggcctctactcactggatgtgggtgaagaaggggaccaggccatcgccgaggcccttgctgcccctagccggtttgtgctaaagccccagagagagggtggaggtaacaacctatatggggaggaaatggtacaggccctgaaacagctgaaggacagtgaggagagggcctcctacatcctcatggagaagatcgaacctgagccttttgagaattgcctgctacggcctggcagccctgcccgagtggtccagtgcatttcagagctgggcatctttggggtctatgtcaggcaggaaaagacactcgtgatgaacaagcacgtggggcatctacttcgaaccaaagccatcgagcatgcagatggtggtgtggcagcgggagtggcagtcctggacaacccataccctgtg

SEQ ID NO: 214 - protein, 1199 aa, Homo sapiens
MIVLPLYSRLDKRFLCLKNIRTFLSTCCEKFGLKRSELFEAFDLFDVQDFGKVIYTLSALSWTPIAQNRGIMPFPTEEESVGDEDIYSGLSDQIDDTVEEDEDLYDCVENEEAEGDEIYEDLMRSEPVSMPPKMTEYDKRCCCLREIQQTEEKYTDTLGSIQQHFLKPLQRFLKPQDIEIIFINIEDLLRVHTHFLKEMKEALGTPGAANLYQVFIKYKERFLVYGRYCSQVESASKHLDRVAAAREDVQMKLEECSQRANNGRFTLRDLLMVPMQRVLKYHLLLQELVKHTQEAMEKENLRLALDAMRDLAQCVNEVKRDNETLRQITNFQLSIENLDQSLAHYGRPKIDGELKITSVERRSKMDRYAFLLDKALLICKRRGDSYDLKDFVNLHSFQVRDDSSGDRDNKKWSHMFLLIEDQGAQGYELFFKTRELKKKWMEQFEMAISNIYPENATANGHDFQMFSFEETTSCKACQMLLRGTFYQGYRCHRCRASAHKECLGRVPPCGRHGQDFPGTMKKDKLHRRAQDKKRNELGLPKMEVFQEYYGLPPPPGAIGPFLRLNPGDIVELTKAEAEQNWWEGRNTSTNEIGWFPCNRVKPYVHGPPQDLSVHLWYAGPMERAGAESILANRSDGTFLVRQRVKDAAEFAISIKYNVEVKHIKIMTAEGLYRITEKKAFRGLTELVEFYQQNSLKDCFKSLDTTLQFPFKEPEKRTISRPAGVGMATNWGSLLQDKQQLEELARQAVDRALAEGVLLRTSQEPTSSEVVSYAPFTLFPSLVPSALLEQAYAVQMDFNLLVDAVSQNAAFLEQTLSSTIKQDDFTARLFDIHKQVLKEGIAQTVFLGLNRSDYMFQRSADGSPALKQIEINTISASFGGLASRTPAVHRHVLSVLSKTKEAGKILSNNPSKGLALGIAKAWELYGSPNALVLLIAQEKERNIFDQRAIENELLARNIHVIRRTFEDISEKGSLDQDRRLFVDGQEIAVVYFRDGYMPRQYSLQNWEARLLLERSHAAKCPDIATQLAGTKKVQQELSRPGMLEMLLPGQPEAVARLRATFAGLYSLDVGEEGDQAIAEALAAPSRFVLKPQREGGGNNLYGEEMVQALKQLKDSEERASYILMEKIEPEPFENCLLRPGSPARVVQCISELGIFGVYVRQEKTLVMNKHVGHLLRTKAIEHADGGVAAGVAVLDNPYPV

SEQ ID NO: 215 - DNA, 2157 bp, Homo sapiens
  source 1..2157: mol_type = genomic DNA
atggaagattcgatggacatggacatgagccccctgaggccccagaactatcttttcggttgtgaactaaaggccgacaaagattatcactttaaggtggataatgatgaaaatgagcaccagttatctttaagaacggtcagtttaggggctggtgcaaaggatgagttgcacattgttgaagcagaggcaatgaattacgaaggcagtccaattaaagtaacactggcaactttgaaaatgtctgtacagccaacggtttcccttgggggctttgaaataacaccaccagtggtcttaaggttgaagtgtggttcagggccagtgcatattagtggacagcacttagtagctgtggaggaagatgcagagtcagaagatgaagaggaggaggatgtgaaactcttaagtatatctggaaagcggtctgcccctggaggtggtagcaaggttccacagaaaaaagtaaaacttgctgctgatgaagatgatgacgatgatgatgaagaggatgatgatgaagatgatgatgatgatgattttgatgatgaggaagctgaagaaaaagcgccagtgaagaaatctatacgagatactccagccaaaaatgcacaaaagtcaaatcagaatggaaaagactcaaaaccatcatcaacaccaagatcaaaaggacaagaatccttcaagaaacaggaaaaaactcctaaaacaccaaaaggacctagttctgtagaagacattaaagcaaaaatgcaagcaagtatagaaaaagagaacaagaacctggttcatggtaatgtgtgtggccggaacatcctgctggcccggctggggttggcagagggcaccagccccttcatcaagctgagtgatcctggcgtgggcctgggcgccctctccagggaggagcgggtggagaggatcccctggctggcccccgaatgcctaccaggtggggccaacagcctaagcaccgccatggacaagtgggggtttggcgccaccctcctggagatctgctttgacggagaggcccctctgcagagccgcagtccctccgagaaggagcatttctaccagaggcagcaccggctgcccgagccctcctgcccacagctggccacactcaccagccagtgtctgacctatgagccaacccagaggccatcattccgcaccatcctgcgtgacctcacccggctgcagccccacaatcttgctgacgtcttgactgtgaacccggactcaccggcgtcggaccctacggttttccacaagcgctatttgaaaaagatccgagatctgggcgagggtcacttcggcaaggtcagcttgtactgctacgatccgaccaacgacggcactggcgagatggtggcggtgaaagccctcaaggcagactgcggcccccagcaccgctcgggctggaagcaggagattgacattctgcgcacgctctaccacgagcacatcatcaagtacaagggctgctgcgaggaccaaggcgagaagtcgctgcagctggtcatggagtacgtgcccctgggcagcctccgagactacctgccccggcacagcatcgggctggcccagctgctgctcttcgcccagcagatctgcgagggcatggcctatctgcactcgcagcactacatccaccgagacctagccgcgcgcaacgtgctgctggacaacgacaggctggtcaagatcggggactttggcctagccaaggccgtgcccgaaggccacgagtactaccgcgtgcgcgaggatggggacagccccgtgttctggtatgccccagagtgcctgaaggagtataagttctactatgcgtcagatgtctggtccttcggggtgaccctgtatgagctgctgacgcactgtgactccagccagagcccccccacgaaattccttgagctcataggcattgctcagggtcagatgacagttctgagactcactgagttgctggaacgaggggagaggctgccacggcccgacaaatgtccctgtgaggtctatcatctcatgaagaactgctgggagacagaggcgtcctttcgcccaaccttcgagaacctcatacccattctgaagacagtccatgagaagtaccaaggccaggccccttcagtgttcagcgtgtgc

SEQ ID NO: 216 - protein, 719 aa, Homo sapiens
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVAVEEDAESEDEEEEDVKLLSISGKRSAPGGGSKVPQKKVKLAADEDDDDDDEEDDDEDDDDDDFDDEEAEEKAPVKKSIRDTPAKNAQKSNQNGKDSKPSSTPRSKGQESFKKQEKTPKTPKGPSSVEDIKAKMQASIEKENKNLVHGNVCGRNILLARLGLAEGTSPFIKLSDPGVGLGALSREERVERIPWLAPECLPGGANSLSTAMDKWGFGATLLEICFDGEAPLQSRSPSEKEHFYQRQHRLPEPSCPQLATLTSQCLTYEPTQRPSFRTILRDLTRLQPHNLADVLTVNPDSPASDPTVFHKRYLKKIRDLGEGHFGKVSLYCYDPTNDGTGEMVAVKALKADCGPQHRSGWKQEIDILRTLYHEHIIKYKGCCEDQGEKSLQLVMEYVPLGSLRDYLPRHSIGLAQLLLFAQQICEGMAYLHSQHYIHRDLAARNVLLDNDRLVKIGDFGLAKAVPEGHEYYRVREDGDSPVFWYAPECLKEYKFYYASDVWSFGVTLYELLTHCDSSQSPPTKFLELIGIAQGQMTVLRLTELLERGERLPRPDKCPCEVYHLMKNCWETEASFRPTFENLIPILKTVHEKYQGQAPSVFSVC

SEQ ID NO: 217 - DNA, 600 bp, Homo sapiens
  source 1..600: mol_type = genomic DNA
atgctcaggctgctcttggctctcaacttattcccttcaattcaagtaacaggaaacaagattttggtgaagcagtcgcccatgcttgtagcgtacgacaatgcggtcaaccttagctgcaagtattcctacaatctcttctcaagggagttccgggcatcccttcacaaaggactggatagtgctgtggaagtctgtgttgtatatgggaattactcccagcagcttcaggtttactcaaaaacggggttcaactgtgatgggaaattgggcaatgaatcagtgacattctacctccagaatttgtatgttaaccaaacagatatttacttctgcaaaattgaagttatgtatcctcctccttacctagagaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggaccctgaaccgtgccctgattctgactttcttttgtggatattggctgccgtctcaagtgggctg

SEQ ID NO: 218 - protein, 200 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLENEKSNGTIIHVKGKHLCPSPLFPGPSKPFWVLVVVGGVLACYSLLVTVAFIIFWDPEPCPDSDFLLWILAAVSSGL

SEQ ID NO: 219 - DNA, 666 bp, Homo sapiens
  source 1..666: mol_type = genomic DNA
atgaaatccggtttgtggtatttctttcttttctgccttcgcatcaaggttttgacgggaaacaagattttggtgaagcagtcgcccatgcttgtagcgtacgacaatgcggtcaaccttagctgcaagtattcctacaatctcttctcaagggagttccgggcatcccttcacaaaggactggatagtgctgtggaagtctgtgttgtatatgggaattactcccagcagcttcaggtttactcaaaaacggggttcaactgtgatgggaaattgggcaatgaatcagtgacattctacctccagaatttgtatgttaaccaaacagatatttacttctgcaaaattgaagttatgtatcctcctccttacctagagaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 220 - protein, 222 aa, Homo sapiens
MKSGLWYFFLFCLRIKVLTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLENEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 221 - DNA, 1215 bp, Homo sapiens
  source 1..1215: mol_type = genomic DNA
atgctccaagcatgtaagatggaaggtttcccccttgttccacctccttctgaagacctggtgccctacgacaccgacctctatcaacgacaaacgcatgagtactacccctatctgagtagtgatggagaaagccattcagaccattactgggatttccatcctcatcacgttcatagtgagtttgagagtttcgctgagaacaacttcacagaattgcagagcgttcaaccaccccagctccagcaactttacaggcacatggaacttgagcagatgcacgttctggatactccaatggttcctccgcatccgtcacttggccatcaagtttcttatctgcctcgaatgtgcctccaatacccctctctcagtccggcgcagcctagtagtgacgaagaagaaggcgagcgacagtcacctcccttggaggtgtcagatggtgaagcggacgggctggaaccaggaccgggacttcttccgggagaaactggttctaaaaaaaagattcggctgtatcaatttcttttggacctcctgagaagcctggcagagttgaagtcaagcctggtcaacgaaagcgaaggtgcggccgggggtgccggcattcctggagttcctggggcgggtgctggcgcgagaggggaagcggaagcccttgggagagaacacgcggcacagcgattgttcccggacaaactccctgaacctttggaggatggacttaaagccccagagtgcacttctggtatgtacaaggaaaccgtgtactccgcatttaatctcctgatgcactaccctccaccaagtggggccggacagcatccacagccgcaaccacccttgcacaaagctaatcagccgccccacggagtgccccagctgagtctttacgagcattttaatagtccacacccgacgccagccccagcagacataagccaaaaacaggtccaccggccactccagaccccagacctgagcggattttatagccttacctcaggctctatgggacagttgccacatactgtaagctggtttactcacccgtctcttatgcttggatccggtgtgccgggtcatcccgctgcaattccacatcctgcaattgtgccgccgtcaggtaaacaagagctccagcctttcgaccgaaacctcaagacccaggcggaatcaaaagcagaaaaagaagcgaagaagccgacaatcaaa

SEQ ID NO: 222 - protein, 405 aa, Homo sapiens
MLQACKMEGFPLVPPPSEDLVPYDTDLYQRQTHEYYPYLSSDGESHSDHYWDFHPHHVHSEFESFAENNFTELQSVQPPQLQQLYRHMELEQMHVLDTPMVPPHPSLGHQVSYLPRMCLQYPSLSPAQPSSDEEEGERQSPPLEVSDGEADGLEPGPGLLPGETGSKKKIRLYQFLLDLLRSLAELKSSLVNESEGAAGGAGIPGVPGAGAGARGEAEALGREHAAQRLFPDKLPEPLEDGLKAPECTSGMYKETVYSAFNLLMHYPPPSGAGQHPQPQPPLHKANQPPHGVPQLSLYEHFNSPHPTPAPADISQKQVHRPLQTPDLSGFYSLTSGSMGQLPHTVSWFTHPSLMLGSGVPGHPAAIPHPAIVPPSGKQELQPFDRNLKTQAESKAEKEAKKPTIK

SEQ ID NO: 223 - DNA, 2817 bp, Homo sapiens
  source 1..2817: mol_type = genomic DNA
atggctggctacgaatacgtgagcccggagcagctggctggctttgataagtacaagaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctggtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctggagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgacctgtcccaggtgtatgagctgctagagaaggactaccgcatggagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgagtaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgcctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagctggaaagggcagtgccttagggacccctgctgcagctgagccagtgacccccaccagcaaagcaggctcaggtgcaccagggggcaccagcaagggccccgccgaggagtccagagtgaggaggcacaagcactcctctgagtcgccagggagggacaaggggaaattgtccaggctcaaacctgccccgccgcccccaccagcagcctctgcagggaaggctggaggaaagccctcgcagagcccgagccaggaggcggccggggaggcagtcctgggcgcaaagacaaaagccacgagtctggttgatgctgtgaacagtgacgctgccaagcccagccagccgggagagggcctcaaaaagcccgtgctcccggccactccaaagccacagtccgccaagccgtcggggacccccatcagcccagcccccgttccctccacgttgccatcagcatcctcggccctggcaggggaccagccgtcttccaccgccttcatccctctcatatcaacccgagtgtctcttcggaaaacccgccagcctccagagcggatcgccagcggtgccatcaccaagggcgtggtcctggacagcaccgaggcgctgtgcctcgccatctctaggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgacagcaggcagtggtccagcggccactcaggacttcagcaagctcctcagttcggtgaaggaaatcagtgacatagtgcagagg

SEQ ID NO: 224 - protein, 939 aa, Homo sapiens
MAGYEYVSPEQLAGFDKYKTTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 225 - DNA, 1791 bp, Synthetic construct
  source 1..1791: mol_type = other DNA
atggatgactacatggagacgctgaaggatgaagaggacgccttgtgggagaatgtggagtgtaaccggcacatgctcagccgctatatcaaccctgccaagctcacgccctacctgcgtcagtgtaaggtcattgatgagcaggatgaagatgaagtgcttaatgcccctatgctgccatccaagatcaaccgagcaggccggctgttggacattctacataccaaggggcaaaggggctatgtggtcttcttggagagcctagaattttattacccagaactgtacaaactggtgactgggaaagagcccactcggagattctccaccattgtggtggaggaaggccacgagggcctcacgcacttcctgatgaacgaggtcatcaagctgcagcagcagatgaaggccaaggacctgcaacgctgcgagctgctggccaggttgcggcagctggaggatgagaagaagcagatgacgctgacgcgcgtggagctgctaaccttccaggagcggtactacaagatgaaggaagagcgggacagctacaatgacgagctggtcaaggtgaaggacgacaactacaacttagccatgcgctacgcacagctcagtgaggagaagaacatggcggtcatgaggagccgagacctccaactcgagatcgatcagctaaagcaccggttgaataagatggaggaggaatgtaagctggagagaaatcagtctctaaaactgaagaatgacattgaaaatcggcccaagaaggagcaggttctggaactggagcgggagaatgaaatgctgaagaccaaaaaccaggagctgcagtccatcatccaggccgggaagcgcagcctgccagactcagacaaggccatcctggacatcttggaacacgaccgcaaggaggccctggaggacaggcaggagctggtcaacaggatctacaacctgcaggaggaggcccgccaggcagaggagctgcgagacaagtacctggaggagaaggaggacctggagctcaagtgctcgaccctgggaaaggactgtgaaatgtacaagcaccgcatgaacacggtcatgctgcagctggaggaggtggagcgggagcgggaccaggccttccactcccgagatgaagctcagacacagtactcgcagtgcttaatcgaaaaggacaagtacaggaagcagatccgcgagctggaggagaagaacgatgagatgaggatcgagatggtgcggcgggaggcctgcatcgtcaacctggagagcaagctgcggcgcctctccaaggacagcaacaacctggaccagagtctgcccaggaacctgccagtaaccatctggctcaatcacaaaggagtgagacagaaacgcctgaatgtctggctgggaattaagaatgaggatgctgctgagaactattttatcaatgaggaagatgaaaacctgccccattatgatgagaaaacctggtttgttgaggatatcaatcgagtacaagcagaggacttgctttatgggaaacctgatggtgcattcttaattcgtgagagtagcaagaaaggatgctatgcttgctctgtggtggccgatggggaagtgaagcactgtgtgatctacagcactgctcggggctatggctttgcagagccctacaacctgtacagctctctgaaggagctagtgctccattaccagcagacatccttggttcagcacaacgactccctcaacgtcaggcttgcctaccctgttcatgcacagatgccctcgctttgcaga

SEQ ID NO: 226 - protein, 597 aa, Synthetic construct
MDDYMETLKDEEDALWENVECNRHMLSRYINPAKLTPYLRQCKVIDEQDEDEVLNAPMLPSKINRAGRLLDILHTKGQRGYVVFLESLEFYYPELYKLVTGKEPTRRFSTIVVEEGHEGLTHFLMNEVIKLQQQMKAKDLQRCELLARLRQLEDEKKQMTLTRVELLTFQERYYKMKEERDSYNDELVKVKDDNYNLAMRYAQLSEEKNMAVMRSRDLQLEIDQLKHRLNKMEEECKLERNQSLKLKNDIENRPKKEQVLELERENEMLKTKNQELQSIIQAGKRSLPDSDKAILDILEHDRKEALEDRQELVNRIYNLQEEARQAEELRDKYLEEKEDLELKCSTLGKDCEMYKHRMNTVMLQLEEVERERDQAFHSRDEAQTQYSQCLIEKDKYRKQIRELEEKNDEMRIEMVRREACIVNLESKLRRLSKDSNNLDQSLPRNLPVTIWLNHKGVRQKRLNVWLGIKNEDAAENYFINEEDENLPHYDEKTWFVEDINRVQAEDLLYGKPDGAFLIRESSKKGCYACSVVADGEVKHCVIYSTARGYGFAEPYNLYSSLKELVLHYQQTSLVQHNDSLNVRLAYPVHAQMPSLCR

SEQ ID NO: 227 - DNA, 1707 bp, Synthetic construct
  source 1..1707: mol_type = other DNA
atggatgactacatggagacgctgaaggatgaagaggacgccttgtgggagaatgtggagtgtaaccggcacatgctcagccgctatatcaaccctgccaagctcacgccctacctgcgtcagtgtaaggtcattgatgagcaggatgaagatgaagtgcttaatgcccctatgctgccatccaagatcaaccgagcaggccggctgttggacattctacataccaaggggcaaaggggctatgtggtcttcttggagagcctagaattttattacccagaactgtacaaactggtgactgggaaagagcccactcggagattctccaccattgtggtggaggaaggccacgagggcctcacgcacttcctgatgaacgaggtcatcaagctgcagcagcagatgaaggccaaggacctgcaacgctgcgagctgctggccaggttgcggcagctggaggatgagaagaagcagatgacgctgacgcgcgtggagctgctaaccttccaggagcggtactacaagatgaaggaagagcgggacagctacaatgacgagctggtcaaggtgaaggacgacaactacaacttagccatgcgctacgcacagctcagtgaggagaagaacatggcggtcatgaggagccgagacctccaactcgagatcgatcagctaaagcaccggttgaataagatggaggaggaatgtaagctggagagaaatcagtctctaaaactgaagaatgacattgaaaatcggcccaagaaggagcaggttctggaactggagcgggagaatgaaatgctgaagaccaaaaaccaggagctgcagtccatcatccaggccgggaagcgcagcctgccagactcagacaaggccatcctggacatcttggaacacgaccgcaaggaggccctggaggacaggcaggagctggtcaacaggatctacaacctgcaggaggaggcccgccaggcagaggagctgcgagacaagtacctggaggagaaggaggacctggagctcaagtgctcgaccctgggaaaggactgtgaaatgtacaagcaccgcatgaacacggtcatgctgcagctggaggaggtggagcgggagcgggaccaggccttccactcccgagatgaagctcagacacagtactcgcagtgcttaatcgaaaaggacaagtacaggaagcagatccgcgagctggaggagaagaacgatgagatgaggatcgagatggtgcggcgggaggcctgcatcgtctggctcaatcacaaaggagtgagacagaaacgcctgaatgtctggctgggaattaagaatgaggatgctgctgagaactattttatcaatgaggaagatgaaaacctgccccattatgatgagaaaacctggtttgttgaggatatcaatcgagtacaagcagaggacttgctttatgggaaacctgatggtgcattcttaattcgtgagagtagcaagaaaggatgctatgcttgctctgtggtggccgatggggaagtgaagcactgtgtgatctacagcactgctcggggctatggctttgcagagccctacaacctgtacagctctctgaaggagctagtgctccattaccagcagacatccttggttcagcacaacgactccctcaacgtcaggcttgcctaccctgttcatgcacagatgccctcgctttgcaga

SEQ ID NO: 228 - protein, 569 aa, Synthetic construct
MDDYMETLKDEEDALWENVECNRHMLSRYINPAKLTPYLRQCKVIDEQDEDEVLNAPMLPSKINRAGRLLDILHTKGQRGYVVFLESLEFYYPELYKLVTGKEPTRRFSTIVVEEGHEGLTHFLMNEVIKLQQQMKAKDLQRCELLARLRQLEDEKKQMTLTRVELLTFQERYYKMKEERDSYNDELVKVKDDNYNLAMRYAQLSEEKNMAVMRSRDLQLEIDQLKHRLNKMEEECKLERNQSLKLKNDIENRPKKEQVLELERENEMLKTKNQELQSIIQAGKRSLPDSDKAILDILEHDRKEALEDRQELVNRIYNLQEEARQAEELRDKYLEEKEDLELKCSTLGKDCEMYKHRMNTVMLQLEEVERERDQAFHSRDEAQTQYSQCLIEKDKYRKQIRELEEKNDEMRIEMVRREACIVWLNHKGVRQKRLNVWLGIKNEDAAENYFINEEDENLPHYDEKTWFVEDINRVQAEDLLYGKPDGAFLIRESSKKGCYACSVVADGEVKHCVIYSTARGYGFAEPYNLYSSLKELVLHYQQTSLVQHNDSLNVRLAYPVHAQMPSLCR

SEQ ID NO: 229 - DNA, 1647 bp, Synthetic construct
  source 1..1647: mol_type = other DNA
atggatgactacatggagacgctgaaggatgaagaggacgccttgtgggagaatgtggagtgtaaccggcacatgctcagccgctatatcaaccctgccaagctcacgccctacctgcgtcagtgtaaggtcattgatgagcaggatgaagatgaagtgcttaatgcccctatgctgccatccaagatcaaccgagcaggccggctgttggacattctacataccaaggggcaaaggggctatgtggtcttcttggagagcctagaattttattacccagaactgtacaaactggtgactgggaaagagcccactcggagattctccaccattgtggtggaggaaggccacgagggcctcacgcacttcctgatgaacgaggtcatcaagctgcagcagcagatgaaggccaaggacctgcaacgctgcgagctgctggccaggttgcggcagctggaggatgagaagaagcagatgacgctgacgcgcgtggagctgctaaccttccaggagcggtactacaagatgaaggaagagcgggacagctacaatgacgagctggtcaaggtgaaggacgacaactacaacttagccatgcgctacgcacagctcagtgaggagaagaacatggcggtcatgaggagccgagacctccaactcgagatcgatcagctaaagcaccggttgaataagatggaggaggaatgtaagctggagagaaatcagtctctaaaactgaagaatgacattgaaaatcggcccaagaaggagcaggttctggaactggagcgggagaatgaaatgctgaagaccaaaaaccaggagctgcagtccatcatccaggccgggaagcgcagcctgccagactcagacaaggccatcctggacatcttggaacacgaccgcaaggaggccctggaggacaggcaggagctggtcaacaggatctacaacctgcaggaggaggcccgccaggcagaggagctgcgagacaagtacctggaggagaaggaggacctggagctcaagtgctcgaccctgggaaaggactgtgaaatgtacaagcaccgcatgaacacggtcatgctgcagctggaggaggtggagcgggagcgggaccaggccttccactcccgagatgaagctcagacacagtactcgcagtgcttaatcgaaaaggacaagtacaggaagcagatccgcgagtggctcaatcacaaaggagtgagacagaaacgcctgaatgtctggctgggaattaagaatgaggatgctgctgagaactattttatcaatgaggaagatgaaaacctgccccattatgatgagaaaacctggtttgttgaggatatcaatcgagtacaagcagaggacttgctttatgggaaacctgatggtgcattcttaattcgtgagagtagcaagaaaggatgctatgcttgctctgtggtggccgatggggaagtgaagcactgtgtgatctacagcactgctcggggctatggctttgcagagccctacaacctgtacagctctctgaaggagctagtgctccattaccagcagacatccttggttcagcacaacgactccctcaacgtcaggcttgcctaccctgttcatgcacagatgccctcgctttgcaga

SEQ ID NO: 230 - protein, 549 aa, Synthetic construct
MDDYMETLKDEEDALWENVECNRHMLSRYINPAKLTPYLRQCKVIDEQDEDEVLNAPMLPSKINRAGRLLDILHTKGQRGYVVFLESLEFYYPELYKLVTGKEPTRRFSTIVVEEGHEGLTHFLMNEVIKLQQQMKAKDLQRCELLARLRQLEDEKKQMTLTRVELLTFQERYYKMKEERDSYNDELVKVKDDNYNLAMRYAQLSEEKNMAVMRSRDLQLEIDQLKHRLNKMEEECKLERNQSLKLKNDIENRPKKEQVLELERENEMLKTKNQELQSIIQAGKRSLPDSDKAILDILEHDRKEALEDRQELVNRIYNLQEEARQAEELRDKYLEEKEDLELKCSTLGKDCEMYKHRMNTVMLQLEEVERERDQAFHSRDEAQTQYSQCLIEKDKYRKQIREWLNHKGVRQKRLNVWLGIKNEDAAENYFINEEDENLPHYDEKTWFVEDINRVQAEDLLYGKPDGAFLIRESSKKGCYACSVVADGEVKHCVIYSTARGYGFAEPYNLYSSLKELVLHYQQTSLVQHNDSLNVRLAYPVHAQMPSLCR

SEQ ID NO: 231 - DNA, 1587 bp, Synthetic construct
  source 1..1587: mol_type = other DNA
atggatgactacatggagacgctgaaggatgaagaggacgccttgtgggagaatgtggagtgtaaccggcacatgctcagccgctatatcaaccctgccaagctcacgccctacctgcgtcagtgtaaggtcattgatgagcaggatgaagatgaagtgcttaatgcccctatgctgccatccaagatcaaccgagcaggccggctgttggacattctacataccaaggggcaaaggggctatgtggtcttcttggagagcctagaattttattacccagaactgtacaaactggtgactgggaaagagcccactcggagattctccaccattgtggtggaggaaggccacgagggcctcacgcacttcctgatgaacgaggtcatcaagctgcagcagcagatgaaggccaaggacctgcaacgctgcgagctgctggccaggttgcggcagctggaggatgagaagaagcagatgacgctgacgcgcgtggagctgctaaccttccaggagcggtactacaagatgaaggaagagcgggacagctacaatgacgagctggtcaaggtgaaggacgacaactacaacttagccatgcgctacgcacagctcagtgaggagaagaacatggcggtcatgaggagccgagacctccaactcgagatcgatcagctaaagcaccggttgaataagatggaggaggaatgtaagctggagagaaatcagtctctaaaactgaagaatgacattgaaaatcggcccaagaaggagcaggttctggaactggagcgggagaatgaaatgctgaagaccaaaaaccaggagctgcagtccatcatccaggccgggaagcgcagcctgccagactcagacaaggccatcctggacatcttggaacacgaccgcaaggaggccctggaggacaggcaggagctggtcaacaggatctacaacctgcaggaggaggcccgccaggcagaggagctgcgagacaagtacctggaggagaaggaggacctggagctcaagtgctcgaccctgggaaaggactgtgaaatgtacaagcaccgcatgaacacggtcatgctgcagctggaggaggtggagcgggagcgggaccaggccttccactcccgagatgaagcttggctcaatcacaaaggagtgagacagaaacgcctgaatgtctggctgggaattaagaatgaggatgctgctgagaactattttatcaatgaggaagatgaaaacctgccccattatgatgagaaaacctggtttgttgaggatatcaatcgagtacaagcagaggacttgctttatgggaaacctgatggtgcattcttaattcgtgagagtagcaagaaaggatgctatgcttgctctgtggtggccgatggggaagtgaagcactgtgtgatctacagcactgctcggggctatggctttgcagagccctacaacctgtacagctctctgaaggagctagtgctccattaccagcagacatccttggttcagcacaacgactccctcaacgtcaggcttgcctaccctgttcatgcacagatgccctcgctttgcaga

SEQ ID NO: 232 - protein, 529 aa, Synthetic construct
MDDYMETLKDEEDALWENVECNRHMLSRYINPAKLTPYLRQCKVIDEQDEDEVLNAPMLPSKINRAGRLLDILHTKGQRGYVVFLESLEFYYPELYKLVTGKEPTRRFSTIVVEEGHEGLTHFLMNEVIKLQQQMKAKDLQRCELLARLRQLEDEKKQMTLTRVELLTFQERYYKMKEERDSYNDELVKVKDDNYNLAMRYAQLSEEKNMAVMRSRDLQLEIDQLKHRLNKMEEECKLERNQSLKLKNDIENRPKKEQVLELERENEMLKTKNQELQSIIQAGKRSLPDSDKAILDILEHDRKEALEDRQELVNRIYNLQEEARQAEELRDKYLEEKEDLELKCSTLGKDCEMYKHRMNTVMLQLEEVERERDQAFHSRDEAWLNHKGVRQKRLNVWLGIKNEDAAENYFINEEDENLPHYDEKTWFVEDINRVQAEDLLYGKPDGAFLIRESSKKGCYACSVVADGEVKHCVIYSTARGYGFAEPYNLYSSLKELVLHYQQTSLVQHNDSLNVRLAYPVHAQMPSLCR

SEQ ID NO: 233 - DNA, 1527 bp, Synthetic construct
  source 1..1527: mol_type = other DNA
atggatgactacatggagacgctgaaggatgaagaggacgccttgtgggagaatgtggagtgtaaccggcacatgctcagccgctatatcaaccctgccaagctcacgccctacctgcgtcagtgtaaggtcattgatgagcaggatgaagatgaagtgcttaatgcccctatgctgccatccaagatcaaccgagcaggccggctgttggacattctacataccaaggggcaaaggggctatgtggtcttcttggagagcctagaattttattacccagaactgtacaaactggtgactgggaaagagcccactcggagattctccaccattgtggtggaggaaggccacgagggcctcacgcacttcctgatgaacgaggtcatcaagctgcagcagcagatgaaggccaaggacctgcaacgctgcgagctgctggccaggttgcggcagctggaggatgagaagaagcagatgacgctgacgcgcgtggagctgctaaccttccaggagcggtactacaagatgaaggaagagcgggacagctacaatgacgagctggtcaaggtgaaggacgacaactacaacttagccatgcgctacgcacagctcagtgaggagaagaacatggcggtcatgaggagccgagacctccaactcgagatcgatcagctaaagcaccggttgaataagatggaggaggaatgtaagctggagagaaatcagtctctaaaactgaagaatgacattgaaaatcggcccaagaaggagcaggttctggaactggagcgggagaatgaaatgctgaagaccaaaaaccaggagctgcagtccatcatccaggccgggaagcgcagcctgccagactcagacaaggccatcctggacatcttggaacacgaccgcaaggaggccctggaggacaggcaggagctggtcaacaggatctacaacctgcaggaggaggcccgccaggcagaggagctgcgagacaagtacctggaggagaaggaggacctggagctcaagtgctcgaccctgggaaaggactgtgaaatgtacaagcaccgcatgaacacggtcatgtggctcaatcacaaaggagtgagacagaaacgcctgaatgtctggctgggaattaagaatgaggatgctgctgagaactattttatcaatgaggaagatgaaaacctgccccattatgatgagaaaacctggtttgttgaggatatcaatcgagtacaagcagaggacttgctttatgggaaacctgatggtgcattcttaattcgtgagagtagcaagaaaggatgctatgcttgctctgtggtggccgatggggaagtgaagcactgtgtgatctacagcactgctcggggctatggctttgcagagccctacaacctgtacagctctctgaaggagctagtgctccattaccagcagacatccttggttcagcacaacgactccctcaacgtcaggcttgcctaccctgttcatgcacagatgccctcgctttgcaga

SEQ ID NO: 234 - protein, 509 aa, Synthetic construct
MDDYMETLKDEEDALWENVECNRHMLSRYINPAKLTPYLRQCKVIDEQDEDEVLNAPMLPSKINRAGRLLDILHTKGQRGYVVFLESLEFYYPELYKLVTGKEPTRRFSTIVVEEGHEGLTHFLMNEVIKLQQQMKAKDLQRCELLARLRQLEDEKKQMTLTRVELLTFQERYYKMKEERDSYNDELVKVKDDNYNLAMRYAQLSEEKNMAVMRSRDLQLEIDQLKHRLNKMEEECKLERNQSLKLKNDIENRPKKEQVLELERENEMLKTKNQELQSIIQAGKRSLPDSDKAILDILEHDRKEALEDRQELVNRIYNLQEEARQAEELRDKYLEEKEDLELKCSTLGKDCEMYKHRMNTVMWLNHKGVRQKRLNVWLGIKNEDAAENYFINEEDENLPHYDEKTWFVEDINRVQAEDLLYGKPDGAFLIRESSKKGCYACSVVADGEVKHCVIYSTARGYGFAEPYNLYSSLKELVLHYQQTSLVQHNDSLNVRLAYPVHAQMPSLCR

SEQ ID NO: 235 - DNA, 1467 bp, Synthetic construct
  source 1..1467: mol_type = other DNA
atggatgactacatggagacgctgaaggatgaagaggacgccttgtgggagaatgtggagtgtaaccggcacatgctcagccgctatatcaaccctgccaagctcacgccctacctgcgtcagtgtaaggtcattgatgagcaggatgaagatgaagtgcttaatgcccctatgctgccatccaagatcaaccgagcaggccggctgttggacattctacataccaaggggcaaaggggctatgtggtcttcttggagagcctagaattttattacccagaactgtacaaactggtgactgggaaagagcccactcggagattctccaccattgtggtggaggaaggccacgagggcctcacgcacttcctgatgaacgaggtcatcaagctgcagcagcagatgaaggccaaggacctgcaacgctgcgagctgctggccaggttgcggcagctggaggatgagaagaagcagatgacgctgacgcgcgtggagctgctaaccttccaggagcggtactacaagatgaaggaagagcgggacagctacaatgacgagctggtcaaggtgaaggacgacaactacaacttagccatgcgctacgcacagctcagtgaggagaagaacatggcggtcatgaggagccgagacctccaactcgagatcgatcagctaaagcaccggttgaataagatggaggaggaatgtaagctggagagaaatcagtctctaaaactgaagaatgacattgaaaatcggcccaagaaggagcaggttctggaactggagcgggagaatgaaatgctgaagaccaaaaaccaggagctgcagtccatcatccaggccgggaagcgcagcctgccagactcagacaaggccatcctggacatcttggaacacgaccgcaaggaggccctggaggacaggcaggagctggtcaacaggatctacaacctgcaggaggaggcccgccaggcagaggagctgcgagacaagtacctggaggagaaggaggacctggagctctggctcaatcacaaaggagtgagacagaaacgcctgaatgtctggctgggaattaagaatgaggatgctgctgagaactattttatcaatgaggaagatgaaaacctgccccattatgatgagaaaacctggtttgttgaggatatcaatcgagtacaagcagaggacttgctttatgggaaacctgatggtgcattcttaattcgtgagagtagcaagaaaggatgctatgcttgctctgtggtggccgatggggaagtgaagcactgtgtgatctacagcactgctcggggctatggctttgcagagccctacaacctgtacagctctctgaaggagctagtgctccattaccagcagacatccttggttcagcacaacgactccctcaacgtcaggcttgcctaccctgttcatgcacagatgccctcgctttgcaga

SEQ ID NO: 236 - protein, 489 aa, Synthetic construct
MDDYMETLKDEEDALWENVECNRHMLSRYINPAKLTPYLRQCKVIDEQDEDEVLNAPMLPSKINRAGRLLDILHTKGQRGYVVFLESLEFYYPELYKLVTGKEPTRRFSTIVVEEGHEGLTHFLMNEVIKLQQQMKAKDLQRCELLARLRQLEDEKKQMTLTRVELLTFQERYYKMKEERDSYNDELVKVKDDNYNLAMRYAQLSEEKNMAVMRSRDLQLEIDQLKHRLNKMEEECKLERNQSLKLKNDIENRPKKEQVLELERENEMLKTKNQELQSIIQAGKRSLPDSDKAILDILEHDRKEALEDRQELVNRIYNLQEEARQAEELRDKYLEEKEDLELWLNHKGVRQKRLNVWLGIKNEDAAENYFINEEDENLPHYDEKTWFVEDINRVQAEDLLYGKPDGAFLIRESSKKGCYACSVVADGEVKHCVIYSTARGYGFAEPYNLYSSLKELVLHYQQTSLVQHNDSLNVRLAYPVHAQMPSLCR

SEQ ID NO: 237 - DNA, 1407 bp, Synthetic construct
  source 1..1407: mol_type = other DNA
atggatgactacatggagacgctgaaggatgaagaggacgccttgtgggagaatgtggagtgtaaccggcacatgctcagccgctatatcaaccctgccaagctcacgccctacctgcgtcagtgtaaggtcattgatgagcaggatgaagatgaagtgcttaatgcccctatgctgccatccaagatcaaccgagcaggccggctgttggacattctacataccaaggggcaaaggggctatgtggtcttcttggagagcctagaattttattacccagaactgtacaaactggtgactgggaaagagcccactcggagattctccaccattgtggtggaggaaggccacgagggcctcacgcacttcctgatgaacgaggtcatcaagctgcagcagcagatgaaggccaaggacctgcaacgctgcgagctgctggccaggttgcggcagctggaggatgagaagaagcagatgacgctgacgcgcgtggagctgctaaccttccaggagcggtactacaagatgaaggaagagcgggacagctacaatgacgagctggtcaaggtgaaggacgacaactacaacttagccatgcgctacgcacagctcagtgaggagaagaacatggcggtcatgaggagccgagacctccaactcgagatcgatcagctaaagcaccggttgaataagatggaggaggaatgtaagctggagagaaatcagtctctaaaactgaagaatgacattgaaaatcggcccaagaaggagcaggttctggaactggagcgggagaatgaaatgctgaagaccaaaaaccaggagctgcagtccatcatccaggccgggaagcgcagcctgccagactcagacaaggccatcctggacatcttggaacacgaccgcaaggaggccctggaggacaggcaggagctggtcaacaggatctacaacctgcaggaggagtggctcaatcacaaaggagtgagacagaaacgcctgaatgtctggctgggaattaagaatgaggatgctgctgagaactattttatcaatgaggaagatgaaaacctgccccattatgatgagaaaacctggtttgttgaggatatcaatcgagtacaagcagaggacttgctttatgggaaacctgatggtgcattcttaattcgtgagagtagcaagaaaggatgctatgcttgctctgtggtggccgatggggaagtgaagcactgtgtgatctacagcactgctcggggctatggctttgcagagccctacaacctgtacagctctctgaaggagctagtgctccattaccagcagacatccttggttcagcacaacgactccctcaacgtcaggcttgcctaccctgttcatgcacagatgccctcgctttgcaga

SEQ ID NO: 238 - protein, 469 aa, Synthetic construct
MDDYMETLKDEEDALWENVECNRHMLSRYINPAKLTPYLRQCKVIDEQDEDEVLNAPMLPSKINRAGRLLDILHTKGQRGYVVFLESLEFYYPELYKLVTGKEPTRRFSTIVVEEGHEGLTHFLMNEVIKLQQQMKAKDLQRCELLARLRQLEDEKKQMTLTRVELLTFQERYYKMKEERDSYNDELVKVKDDNYNLAMRYAQLSEEKNMAVMRSRDLQLEIDQLKHRLNKMEEECKLERNQSLKLKNDIENRPKKEQVLELERENEMLKTKNQELQSIIQAGKRSLPDSDKAILDILEHDRKEALEDRQELVNRIYNLQEEWLNHKGVRQKRLNVWLGIKNEDAAENYFINEEDENLPHYDEKTWFVEDINRVQAEDLLYGKPDGAFLIRESSKKGCYACSVVADGEVKHCVIYSTARGYGFAEPYNLYSSLKELVLHYQQTSLVQHNDSLNVRLAYPVHAQMPSLCR

SEQ ID NO: 239 - DNA, 1347 bp, Synthetic construct
  source 1..1347: mol_type = other DNA
atggatgactacatggagacgctgaaggatgaagaggacgccttgtgggagaatgtggagtgtaaccggcacatgctcagccgctatatcaaccctgccaagctcacgccctacctgcgtcagtgtaaggtcattgatgagcaggatgaagatgaagtgcttaatgcccctatgctgccatccaagatcaaccgagcaggccggctgttggacattctacataccaaggggcaaaggggctatgtggtcttcttggagagcctagaattttattacccagaactgtacaaactggtgactgggaaagagcccactcggagattctccaccattgtggtggaggaaggccacgagggcctcacgcacttcctgatgaacgaggtcatcaagctgcagcagcagatgaaggccaaggacctgcaacgctgcgagctgctggccaggttgcggcagctggaggatgagaagaagcagatgacgctgacgcgcgtggagctgctaaccttccaggagcggtactacaagatgaaggaagagcgggacagctacaatgacgagctggtcaaggtgaaggacgacaactacaacttagccatgcgctacgcacagctcagtgaggagaagaacatggcggtcatgaggagccgagacctccaactcgagatcgatcagctaaagcaccggttgaataagatggaggaggaatgtaagctggagagaaatcagtctctaaaactgaagaatgacattgaaaatcggcccaagaaggagcaggttctggaactggagcgggagaatgaaatgctgaagaccaaaaaccaggagctgcagtccatcatccaggccgggaagcgcagcctgccagactcagacaaggccatcctggacatcttggaacacgaccgctggctcaatcacaaaggagtgagacagaaacgcctgaatgtctggctgggaattaagaatgaggatgctgctgagaactattttatcaatgaggaagatgaaaacctgccccattatgatgagaaaacctggtttgttgaggatatcaatcgagtacaagcagaggacttgctttatgggaaacctgatggtgcattcttaattcgtgagagtagcaagaaaggatgctatgcttgctctgtggtggccgatggggaagtgaagcactgtgtgatctacagcactgctcggggctatggctttgcagagccctacaacctgtacagctctctgaaggagctagtgctccattaccagcagacatccttggttcagcacaacgactccctcaacgtcaggcttgcctaccctgttcatgcacagatgccctcgctttgcaga

SEQ ID NO: 240 - protein, 449 aa, Synthetic construct
MDDYMETLKDEEDALWENVECNRHMLSRYINPAKLTPYLRQCKVIDEQDEDEVLNAPMLPSKINRAGRLLDILHTKGQRGYVVFLESLEFYYPELYKLVTGKEPTRRFSTIVVEEGHEGLTHFLMNEVIKLQQQMKAKDLQRCELLARLRQLEDEKKQMTLTRVELLTFQERYYKMKEERDSYNDELVKVKDDNYNLAMRYAQLSEEKNMAVMRSRDLQLEIDQLKHRLNKMEEECKLERNQSLKLKNDIENRPKKEQVLELERENEMLKTKNQELQSIIQAGKRSLPDSDKAILDILEHDRWLNHKGVRQKRLNVWLGIKNEDAAENYFINEEDENLPHYDEKTWFVEDINRVQAEDLLYGKPDGAFLIRESSKKGCYACSVVADGEVKHCVIYSTARGYGFAEPYNLYSSLKELVLHYQQTSLVQHNDSLNVRLAYPVHAQMPSLCR

SEQ ID NO: 241 - DNA, 1287 bp, Synthetic construct
  source 1..1287: mol_type = other DNA
atggatgactacatggagacgctgaaggatgaagaggacgccttgtgggagaatgtggagtgtaaccggcacatgctcagccgctatatcaaccctgccaagctcacgccctacctgcgtcagtgtaaggtcattgatgagcaggatgaagatgaagtgcttaatgcccctatgctgccatccaagatcaaccgagcaggccggctgttggacattctacataccaaggggcaaaggggctatgtggtcttcttggagagcctagaattttattacccagaactgtacaaactggtgactgggaaagagcccactcggagattctccaccattgtggtggaggaaggccacgagggcctcacgcacttcctgatgaacgaggtcatcaagctgcagcagcagatgaaggccaaggacctgcaacgctgcgagctgctggccaggttgcggcagctggaggatgagaagaagcagatgacgctgacgcgcgtggagctgctaaccttccaggagcggtactacaagatgaaggaagagcgggacagctacaatgacgagctggtcaaggtgaaggacgacaactacaacttagccatgcgctacgcacagctcagtgaggagaagaacatggcggtcatgaggagccgagacctccaactcgagatcgatcagctaaagcaccggttgaataagatggaggaggaatgtaagctggagagaaatcagtctctaaaactgaagaatgacattgaaaatcggcccaagaaggagcaggttctggaactggagcgggagaatgaaatgctgaagaccaaaaaccaggagctgcagtccatcatccaggcctggctcaatcacaaaggagtgagacagaaacgcctgaatgtctggctgggaattaagaatgaggatgctgctgagaactattttatcaatgaggaagatgaaaacctgccccattatgatgagaaaacctggtttgttgaggatatcaatcgagtacaagcagaggacttgctttatgggaaacctgatggtgcattcttaattcgtgagagtagcaagaaaggatgctatgcttgctctgtggtggccgatggggaagtgaagcactgtgtgatctacagcactgctcggggctatggctttgcagagccctacaacctgtacagctctctgaaggagctagtgctccattaccagcagacatccttggttcagcacaacgactccctcaacgtcaggcttgcctaccctgttcatgcacagatgccctcgctttgcaga

SEQ ID NO: 242 - protein, 429 aa, Synthetic construct
MDDYMETLKDEEDALWENVECNRHMLSRYINPAKLTPYLRQCKVIDEQDEDEVLNAPMLPSKINRAGRLLDILHTKGQRGYVVFLESLEFYYPELYKLVTGKEPTRRFSTIVVEEGHEGLTHFLMNEVIKLQQQMKAKDLQRCELLARLRQLEDEKKQMTLTRVELLTFQERYYKMKEERDSYNDELVKVKDDNYNLAMRYAQLSEEKNMAVMRSRDLQLEIDQLKHRLNKMEEECKLERNQSLKLKNDIENRPKKEQVLELERENEMLKTKNQELQSIIQAWLNHKGVRQKRLNVWLGIKNEDAAENYFINEEDENLPHYDEKTWFVEDINRVQAEDLLYGKPDGAFLIRESSKKGCYACSVVADGEVKHCVIYSTARGYGFAEPYNLYSSLKELVLHYQQTSLVQHNDSLNVRLAYPVHAQMPSLCR

SEQ ID NO: 243 - DNA, 1227 bp, Synthetic construct
  source 1..1227: mol_type = other DNA
atggatgactacatggagacgctgaaggatgaagaggacgccttgtgggagaatgtggagtgtaaccggcacatgctcagccgctatatcaaccctgccaagctcacgccctacctgcgtcagtgtaaggtcattgatgagcaggatgaagatgaagtgcttaatgcccctatgctgccatccaagatcaaccgagcaggccggctgttggacattctacataccaaggggcaaaggggctatgtggtcttcttggagagcctagaattttattacccagaactgtacaaactggtgactgggaaagagcccactcggagattctccaccattgtggtggaggaaggccacgagggcctcacgcacttcctgatgaacgaggtcatcaagctgcagcagcagatgaaggccaaggacctgcaacgctgcgagctgctggccaggttgcggcagctggaggatgagaagaagcagatgacgctgacgcgcgtggagctgctaaccttccaggagcggtactacaagatgaaggaagagcgggacagctacaatgacgagctggtcaaggtgaaggacgacaactacaacttagccatgcgctacgcacagctcagtgaggagaagaacatggcggtcatgaggagccgagacctccaactcgagatcgatcagctaaagcaccggttgaataagatggaggaggaatgtaagctggagagaaatcagtctctaaaactgaagaatgacattgaaaatcggcccaagaaggagcaggttctggaactgtggctcaatcacaaaggagtgagacagaaacgcctgaatgtctggctgggaattaagaatgaggatgctgctgagaactattttatcaatgaggaagatgaaaacctgccccattatgatgagaaaacctggtttgttgaggatatcaatcgagtacaagcagaggacttgctttatgggaaacctgatggtgcattcttaattcgtgagagtagcaagaaaggatgctatgcttgctctgtggtggccgatggggaagtgaagcactgtgtgatctacagcactgctcggggctatggctttgcagagccctacaacctgtacagctctctgaaggagctagtgctccattaccagcagacatccttggttcagcacaacgactccctcaacgtcaggcttgcctaccctgttcatgcacagatgccctcgctttgcaga

SEQ ID NO: 244 - protein, 409 aa, Synthetic construct
MDDYMETLKDEEDALWENVECNRHMLSRYINPAKLTPYLRQCKVIDEQDEDEVLNAPMLPSKINRAGRLLDILHTKGQRGYVVFLESLEFYYPELYKLVTGKEPTRRFSTIVVEEGHEGLTHFLMNEVIKLQQQMKAKDLQRCELLARLRQLEDEKKQMTLTRVELLTFQERYYKMKEERDSYNDELVKVKDDNYNLAMRYAQLSEEKNMAVMRSRDLQLEIDQLKHRLNKMEEECKLERNQSLKLKNDIENRPKKEQVLELWLNHKGVRQKRLNVWLGIKNEDAAENYFINEEDENLPHYDEKTWFVEDINRVQAEDLLYGKPDGAFLIRESSKKGCYACSVVADGEVKHCVIYSTARGYGFAEPYNLYSSLKELVLHYQQTSLVQHNDSLNVRLAYPVHAQMPSLCR

SEQ ID NO: 245 - DNA, 1167 bp, Synthetic construct
  source 1..1167: mol_type = other DNA
atggatgactacatggagacgctgaaggatgaagaggacgccttgtgggagaatgtggagtgtaaccggcacatgctcagccgctatatcaaccctgccaagctcacgccctacctgcgtcagtgtaaggtcattgatgagcaggatgaagatgaagtgcttaatgcccctatgctgccatccaagatcaaccgagcaggccggctgttggacattctacataccaaggggcaaaggggctatgtggtcttcttggagagcctagaattttattacccagaactgtacaaactggtgactgggaaagagcccactcggagattctccaccattgtggtggaggaaggccacgagggcctcacgcacttcctgatgaacgaggtcatcaagctgcagcagcagatgaaggccaaggacctgcaacgctgcgagctgctggccaggttgcggcagctggaggatgagaagaagcagatgacgctgacgcgcgtggagctgctaaccttccaggagcggtactacaagatgaaggaagagcgggacagctacaatgacgagctggtcaaggtgaaggacgacaactacaacttagccatgcgctacgcacagctcagtgaggagaagaacatggcggtcatgaggagccgagacctccaactcgagatcgatcagctaaagcaccggttgaataagatggaggaggaatgtaagctggagagaaatcagtggctcaatcacaaaggagtgagacagaaacgcctgaatgtctggctgggaattaagaatgaggatgctgctgagaactattttatcaatgaggaagatgaaaacctgccccattatgatgagaaaacctggtttgttgaggatatcaatcgagtacaagcagaggacttgctttatgggaaacctgatggtgcattcttaattcgtgagagtagcaagaaaggatgctatgcttgctctgtggtggccgatggggaagtgaagcactgtgtgatctacagcactgctcggggctatggctttgcagagccctacaacctgtacagctctctgaaggagctagtgctccattaccagcagacatccttggttcagcacaacgactccctcaacgtcaggcttgcctaccctgttcatgcacagatgccctcgctttgcaga

SEQ ID NO: 246 - protein, 389 aa, Synthetic construct
MDDYMETLKDEEDALWENVECNRHMLSRYINPAKLTPYLRQCKVIDEQDEDEVLNAPMLPSKINRAGRLLDILHTKGQRGYVVFLESLEFYYPELYKLVTGKEPTRRFSTIVVEEGHEGLTHFLMNEVIKLQQQMKAKDLQRCELLARLRQLEDEKKQMTLTRVELLTFQERYYKMKEERDSYNDELVKVKDDNYNLAMRYAQLSEEKNMAVMRSRDLQLEIDQLKHRLNKMEEECKLERNQWLNHKGVRQKRLNVWLGIKNEDAAENYFINEEDENLPHYDEKTWFVEDINRVQAEDLLYGKPDGAFLIRESSKKGCYACSVVADGEVKHCVIYSTARGYGFAEPYNLYSSLKELVLHYQQTSLVQHNDSLNVRLAYPVHAQMPSLCR

SEQ ID NO: 247 - DNA, 1107 bp, Synthetic construct
  source 1..1107: mol_type = other DNA
atggatgactacatggagacgctgaaggatgaagaggacgccttgtgggagaatgtggagtgtaaccggcacatgctcagccgctatatcaaccctgccaagctcacgccctacctgcgtcagtgtaaggtcattgatgagcaggatgaagatgaagtgcttaatgcccctatgctgccatccaagatcaaccgagcaggccggctgttggacattctacataccaaggggcaaaggggctatgtggtcttcttggagagcctagaattttattacccagaactgtacaaactggtgactgggaaagagcccactcggagattctccaccattgtggtggaggaaggccacgagggcctcacgcacttcctgatgaacgaggtcatcaagctgcagcagcagatgaaggccaaggacctgcaacgctgcgagctgctggccaggttgcggcagctggaggatgagaagaagcagatgacgctgacgcgcgtggagctgctaaccttccaggagcggtactacaagatgaaggaagagcgggacagctacaatgacgagctggtcaaggtgaaggacgacaactacaacttagccatgcgctacgcacagctcagtgaggagaagaacatggcggtcatgaggagccgagacctccaactcgagatctggctcaatcacaaaggagtgagacagaaacgcctgaatgtctggctgggaattaagaatgaggatgctgctgagaactattttatcaatgaggaagatgaaaacctgccccattatgatgagaaaacctggtttgttgaggatatcaatcgagtacaagcagaggacttgctttatgggaaacctgatggtgcattcttaattcgtgagagtagcaagaaaggatgctatgcttgctctgtggtggccgatggggaagtgaagcactgtgtgatctacagcactgctcggggctatggctttgcagagccctacaacctgtacagctctctgaaggagctagtgctccattaccagcagacatccttggttcagcacaacgactccctcaacgtcaggcttgcctaccctgttcatgcacagatgccctcgctttgcaga

SEQ ID NO: 248 - protein, 369 aa, Synthetic construct
MDDYMETLKDEEDALWENVECNRHMLSRYINPAKLTPYLRQCKVIDEQDEDEVLNAPMLPSKINRAGRLLDILHTKGQRGYVVFLESLEFYYPELYKLVTGKEPTRRFSTIVVEEGHEGLTHFLMNEVIKLQQQMKAKDLQRCELLARLRQLEDEKKQMTLTRVELLTFQERYYKMKEERDSYNDELVKVKDDNYNLAMRYAQLSEEKNMAVMRSRDLQLEIWLNHKGVRQKRLNVWLGIKNEDAAENYFINEEDENLPHYDEKTWFVEDINRVQAEDLLYGKPDGAFLIRESSKKGCYACSVVADGEVKHCVIYSTARGYGFAEPYNLYSSLKELVLHYQQTSLVQHNDSLNVRLAYPVHAQMPSLCR

SEQ ID NO: 249 - DNA, 1047 bp, Synthetic construct
  source 1..1047: mol_type = other DNA
atggatgactacatggagacgctgaaggatgaagaggacgccttgtgggagaatgtggagtgtaaccggcacatgctcagccgctatatcaaccctgccaagctcacgccctacctgcgtcagtgtaaggtcattgatgagcaggatgaagatgaagtgcttaatgcccctatgctgccatccaagatcaaccgagcaggccggctgttggacattctacataccaaggggcaaaggggctatgtggtcttcttggagagcctagaattttattacccagaactgtacaaactggtgactgggaaagagcccactcggagattctccaccattgtggtggaggaaggccacgagggcctcacgcacttcctgatgaacgaggtcatcaagctgcagcagcagatgaaggccaaggacctgcaacgctgcgagctgctggccaggttgcggcagctggaggatgagaagaagcagatgacgctgacgcgcgtggagctgctaaccttccaggagcggtactacaagatgaaggaagagcgggacagctacaatgacgagctggtcaaggtgaaggacgacaactacaacttagccatgcgctacgcatggctcaatcacaaaggagtgagacagaaacgcctgaatgtctggctgggaattaagaatgaggatgctgctgagaactattttatcaatgaggaagatgaaaacctgccccattatgatgagaaaacctggtttgttgaggatatcaatcgagtacaagcagaggacttgctttatgggaaacctgatggtgcattcttaattcgtgagagtagcaagaaaggatgctatgcttgctctgtggtggccgatggggaagtgaagcactgtgtgatctacagcactgctcggggctatggctttgcagagccctacaacctgtacagctctctgaaggagctagtgctccattaccagcagacatccttggttcagcacaacgactccctcaacgtcaggcttgcctaccctgttcatgcacagatgccctcgctttgcaga

SEQ ID NO: 250 - protein, 349 aa, Synthetic construct
MDDYMETLKDEEDALWENVECNRHMLSRYINPAKLTPYLRQCKVIDEQDEDEVLNAPMLPSKINRAGRLLDILHTKGQRGYVVFLESLEFYYPELYKLVTGKEPTRRFSTIVVEEGHEGLTHFLMNEVIKLQQQMKAKDLQRCELLARLRQLEDEKKQMTLTRVELLTFQERYYKMKEERDSYNDELVKVKDDNYNLAMRYAWLNHKGVRQKRLNVWLGIKNEDAAENYFINEEDENLPHYDEKTWFVEDINRVQAEDLLYGKPDGAFLIRESSKKGCYACSVVADGEVKHCVIYSTARGYGFAEPYNLYSSLKELVLHYQQTSLVQHNDSLNVRLAYPVHAQMPSLCR

SEQ ID NO: 251 - DNA, 987 bp, Synthetic construct
  source 1..987: mol_type = other DNA
atggatgactacatggagacgctgaaggatgaagaggacgccttgtgggagaatgtggagtgtaaccggcacatgctcagccgctatatcaaccctgccaagctcacgccctacctgcgtcagtgtaaggtcattgatgagcaggatgaagatgaagtgcttaatgcccctatgctgccatccaagatcaaccgagcaggccggctgttggacattctacataccaaggggcaaaggggctatgtggtcttcttggagagcctagaattttattacccagaactgtacaaactggtgactgggaaagagcccactcggagattctccaccattgtggtggaggaaggccacgagggcctcacgcacttcctgatgaacgaggtcatcaagctgcagcagcagatgaaggccaaggacctgcaacgctgcgagctgctggccaggttgcggcagctggaggatgagaagaagcagatgacgctgacgcgcgtggagctgctaaccttccaggagcggtactacaagatgaaggaagagcgggacagctggctcaatcacaaaggagtgagacagaaacgcctgaatgtctggctgggaattaagaatgaggatgctgctgagaactattttatcaatgaggaagatgaaaacctgccccattatgatgagaaaacctggtttgttgaggatatcaatcgagtacaagcagaggacttgctttatgggaaacctgatggtgcattcttaattcgtgagagtagcaagaaaggatgctatgcttgctctgtggtggccgatggggaagtgaagcactgtgtgatctacagcactgctcggggctatggctttgcagagccctacaacctgtacagctctctgaaggagctagtgctccattaccagcagacatccttggttcagcacaacgactccctcaacgtcaggcttgcctaccctgttcatgcacagatgccctcgctttgcaga

SEQ ID NO: 252 - protein, 329 aa, Synthetic construct
MDDYMETLKDEEDALWENVECNRHMLSRYINPAKLTPYLRQCKVIDEQDEDEVLNAPMLPSKINRAGRLLDILHTKGQRGYVVFLESLEFYYPELYKLVTGKEPTRRFSTIVVEEGHEGLTHFLMNEVIKLQQQMKAKDLQRCELLARLRQLEDEKKQMTLTRVELLTFQERYYKMKEERDSWLNHKGVRQKRLNVWLGIKNEDAAENYFINEEDENLPHYDEKTWFVEDINRVQAEDLLYGKPDGAFLIRESSKKGCYACSVVADGEVKHCVIYSTARGYGFAEPYNLYSSLKELVLHYQQTSLVQHNDSLNVRLAYPVHAQMPSLCR

SEQ ID NO: 253 - DNA, 927 bp, Synthetic construct
  source 1..927: mol_type = other DNA
atggatgactacatggagacgctgaaggatgaagaggacgccttgtgggagaatgtggagtgtaaccggcacatgctcagccgctatatcaaccctgccaagctcacgccctacctgcgtcagtgtaaggtcattgatgagcaggatgaagatgaagtgcttaatgcccctatgctgccatccaagatcaaccgagcaggccggctgttggacattctacataccaaggggcaaaggggctatgtggtcttcttggagagcctagaattttattacccagaactgtacaaactggtgactgggaaagagcccactcggagattctccaccattgtggtggaggaaggccacgagggcctcacgcacttcctgatgaacgaggtcatcaagctgcagcagcagatgaaggccaaggacctgcaacgctgcgagctgctggccaggttgcggcagctggaggatgagaagaagcagatgacgctgacgtggctcaatcacaaaggagtgagacagaaacgcctgaatgtctggctgggaattaagaatgaggatgctgctgagaactattttatcaatgaggaagatgaaaacctgccccattatgatgagaaaacctggtttgttgaggatatcaatcgagtacaagcagaggacttgctttatgggaaacctgatggtgcattcttaattcgtgagagtagcaagaaaggatgctatgcttgctctgtggtggccgatggggaagtgaagcactgtgtgatctacagcactgctcggggctatggctttgcagagccctacaacctgtacagctctctgaaggagctagtgctccattaccagcagacatccttggttcagcacaacgactccctcaacgtcaggcttgcctaccctgttcatgcacagatgccctcgctttgcaga

SEQ ID NO: 254 - protein, 309 aa, Synthetic construct
MDDYMETLKDEEDALWENVECNRHMLSRYINPAKLTPYLRQCKVIDEQDEDEVLNAPMLPSKINRAGRLLDILHTKGQRGYVVFLESLEFYYPELYKLVTGKEPTRRFSTIVVEEGHEGLTHFLMNEVIKLQQQMKAKDLQRCELLARLRQLEDEKKQMTLTWLNHKGVRQKRLNVWLGIKNEDAAENYFINEEDENLPHYDEKTWFVEDINRVQAEDLLYGKPDGAFLIRESSKKGCYACSVVADGEVKHCVIYSTARGYGFAEPYNLYSSLKELVLHYQQTSLVQHNDSLNVRLAYPVHAQMPSLCR

SEQ ID NO: 255 - DNA, 867 bp, Synthetic construct
  source 1..867: mol_type = other DNA
atggatgactacatggagacgctgaaggatgaagaggacgccttgtgggagaatgtggagtgtaaccggcacatgctcagccgctatatcaaccctgccaagctcacgccctacctgcgtcagtgtaaggtcattgatgagcaggatgaagatgaagtgcttaatgcccctatgctgccatccaagatcaaccgagcaggccggctgttggacattctacataccaaggggcaaaggggctatgtggtcttcttggagagcctagaattttattacccagaactgtacaaactggtgactgggaaagagcccactcggagattctccaccattgtggtggaggaaggccacgagggcctcacgcacttcctgatgaacgaggtcatcaagctgcagcagcagatgaaggccaaggacctgcaacgctggctcaatcacaaaggagtgagacagaaacgcctgaatgtctggctgggaattaagaatgaggatgctgctgagaactattttatcaatgaggaagatgaaaacctgccccattatgatgagaaaacctggtttgttgaggatatcaatcgagtacaagcagaggacttgctttatgggaaacctgatggtgcattcttaattcgtgagagtagcaagaaaggatgctatgcttgctctgtggtggccgatggggaagtgaagcactgtgtgatctacagcactgctcggggctatggctttgcagagccctacaacctgtacagctctctgaaggagctagtgctccattaccagcagacatccttggttcagcacaacgactccctcaacgtcaggcttgcctaccctgttcatgcacagatgccctcgctttgcaga

SEQ ID NO: 256 - protein, 289 aa, Synthetic construct
MDDYMETLKDEEDALWENVECNRHMLSRYINPAKLTPYLRQCKVIDEQDEDEVLNAPMLPSKINRAGRLLDILHTKGQRGYVVFLESLEFYYPELYKLVTGKEPTRRFSTIVVEEGHEGLTHFLMNEVIKLQQQMKAKDLQRWLNHKGVRQKRLNVWLGIKNEDAAENYFINEEDENLPHYDEKTWFVEDINRVQAEDLLYGKPDGAFLIRESSKKGCYACSVVADGEVKHCVIYSTARGYGFAEPYNLYSSLKELVLHYQQTSLVQHNDSLNVRLAYPVHAQMPSLCR

SEQ ID NO: 257 - skipped

SEQ ID NO: 258 - protein, 585 aa, Synthetic construct
MPGGGPEMDDYMETLKDEEDALWENVECNRHMLSRYINPAKLTPYLRQCKVIDEQDEDEVLNAPMLPSKINRAGRLLDILHTKGQRGYVVFLESLEFYYPELYKLVTGKEPTRRFSTIVVEEGHEGLTHFLMNEVIKLQQQMKAKDLQRCELLARLRQLEDEKKQMTLTRVELLTFQERYYKMKEERDSYNDELVKVKDDNYNLAMRYAQLSEEKNMAVMRSRDLQLEIDQLKHRLNKMEEECKLERNQSLKLKNDIENRPKKEQVLELERENEMLKTKNQELQSIIQAGKRSLPDSDKAILDILEHDRKEALEDRQELVNRIYNLQEEARQAEELRDKYLEEKEDLELKCSTLGKDCEMYKHRMNTVMLQLEEVERERDQAFHSRDEAQTQYSQCLIEKDKYRKQIRELEEKNDEMRIEMVRREACIVNLESKLRRLSKDSNNLDQSLPRNLPVTIISQDFGDASPRTNGQEADDSSTSEESPEDSKYFLPYHPPQRRMNLKGIQLQRAKSPISLKRTSDFQAKGHEEEGTDASPSSCGSLPITNSFTKMQPPRSRSSIMSITAEPPGNDSIVRRYKEDAPHRS

SEQ ID NO: 259 - skipped

SEQ ID NO: 260 - protein, 147 aa, Synthetic construct
WLNHKGVRQKRLNVWLGIKNEDADENYFINEEDENLPHYDEKTWFVEDINRVQAEDLLYGKPDGAFLIRESSKKGCYACSVVADGEVKHCVIYSTARGYGFAEPYNLYSSLKELVLHYQQTSLVQHNDSLNVRLAYPVHAQMPSLCR

SEQ ID NO: 261 - protein, 93 aa, Homo sapiens
NDDECWSVLEGFRVTLTSVIDPSRITPYLRQCKVLNPDDEEQVLSDPNLVIRKRKVGVLLDILQRTGHKGYVAFLESLELYYPQLYKKVTGKE

SEQ ID NO: 262 - protein, 93 aa, Homo sapiens
EEDALWERIEGVRHRLARALNPAKLTPYLRQCRVIDEQDEEEVLSTYRFPCRVNRTGRLMDILRCRGKRGYEAFLEALEFYYPEHFTLLTGQE

SEQ ID NO: 263 - protein, 93 aa, Homo sapiens
EEDALWENVECNRHMLSRYINPAKLTPYLRQCKVIDEQDEDEVLNAPMLPSKINRAGRLLDILHTKGQRGYVVFLESLEFYYPELYKLVTGKE

SEQ ID NO: 264 - protein, 93 aa, Homo sapiens
DEETLWEMMESHRHRIVRCICPSRLTPYLRQAKVLCQLDEEEVLHSPRLTNSAMRAGHLLDLLKTRGKNGAIAFLESLKFHNPDVYTLVTGLQ

SEQ ID NO: 265 - protein, 69 aa, Homo sapiens
IERERKKLLEILQHDPDSILDTLTSRRLISEEEYETLENVTDLLKKSRKLLILVQKKGEATCQHFLKCL

SEQ ID NO: 266 - protein, 83 aa, Homo sapiens
FVKENHRQLQARMGDLKGVLDDLQDNEVLTENEKELVEQEKTRQSKNEALLSMVEKKGDLALDVLFRSISERDPYLVSYLRQQ

SEQ ID NO: 267 - protein, 83 aa, Homo sapiens
VLKEKRKLFIHSMGEGTINGLLDELLQTRVLNQEEMEKVKRENATVMDKTRALIDSVIPKGAQACQICITYICEEDSYLAETL

SEQ ID NO: 268 - protein, 83 aa, Homo sapiens
VLKEKRKQFIRSVGEGTINGLLGELLETRVLSQEEIEIVKCENATVMDKARALLDSVIRKGAPACQICITYICEEDSHLAGTL

SEQ ID NO: 269 - protein, 82 aa, Homo sapiens
LRKKRRIFIHSVGAGTINALLDCLLEDEVISQEDMNKVRDENDTVMDKARVLIDLVTGKGPKSCCKFIKHLCEEDPQLASKM

SEQ ID NO: 270 - protein, 92 aa, Homo sapiens
DRLVQDTPFLTGHGRLSEQQVDRIILQLNRYYPQILTNKEAEKFRNPKASLRVRLCDLLSHLQRSGERDCQEFYRALYIHAQPLHSRLPSRH

SEQ ID NO: 271 - protein, 83 aa, Homo sapiens
VLKEKRKLFIRSMGEGTINGLLDELLQTRVLNKEEMEKVKRENATVMDKTRALIDSVIPKGAQACQICITYICEEDSYLAGTL

SEQ ID NO: 272 - protein, 87 aa, Homo sapiens
MHPHHQETLKKNRVVLAKQLLLSELLEHLLEKDIITLEMRELIQAKVGSFSQNVELLNLLPKRGPQAFDAFCEALRETKQGHLEDML

SEQ ID NO: 273 - protein, 77 aa, Homo sapiens
NHRKKPLKVLESLGKDFLTGVLDNLVEQNVLNWKEEEKKKYYDAKTEDKVRVMADSMQEKQRMAGQMLLQTFFNIDQ

SEQ ID NO: 274 - protein, 80 aa, Homo sapiens
NHKKKTVKMLEYLGKDVLHGVFNYLAKHDVLTLKEEEKKKYYDTKIEDKALILVDSLRKNRVAHQMFTQTLLNMDQKITS

SEQ ID NO: 275 - protein, 74 aa, Homo sapiens
VEELQVDQLWDALLSRELFRPHMIEDIQRAGSGSRRDQARQLIIDLETRGSQALPLFISCLEDTGQDMLASFLR

SEQ ID NO: 276 - protein, 92 aa, Homo sapiens
MASDDLSLIRKNRMALFQQLTCVLPILDNLLKANVINKQEHDIIKQKTQIPLQARELIDTILVKGNAAANIFKNCLKEIDSTLYKNLFVDKN

SEQ ID NO: 277 - protein, 90 aa, Homo sapiens
SNDLLLIRKNRMALFQHLTCVIPILDSLLTAGIINEQEHDVIKQKTQTSLQARELIDTILVKGNIAATVFRNSLQEAEAVLYEHLFVQQD

SEQ ID NO: 278 - protein, 94 aa, Homo sapiens
MEARDKQVLRSLRLELGAEVLVEGLVLQYLYQEGILTENHIQEINAQTTGLRKTMLLLDILPSRGPKAFDTFLDSLQEFPWVREKLKKAREEAM

SEQ ID NO: 279 - protein, 83 aa, Homo sapiens
SDRQINQLAQRLGPEWEPMVLSLGLSQTDIYRCKANHPHNVQSQVVEAFIRWRQRFGKQATFQSLHNGLRAVEVDPSLLLHML

SEQ ID NO: 280 - protein, 85 aa, Homo sapiens
QLLKSNRELLVTHIRNTQCLVDNLLKNDYFSAEDAEIVCACPTQPDKVRKILDLVQSKGEEVSEFFLYLLQQLADAYVDLRPWLL

SEQ ID NO: 281 - protein, 86 aa, Homo sapiens
FQAQRSQLVELLVSGSLEGFESVLDWLLSWEVLSWEDYEGFHLLGQPLSHLARRLLDTVWNKGTWACQKLIAAAQEAQADSQSPKL

SEQ ID NO: 282 - protein, 81 aa, Homo sapiens
LQSHRPAIVRRLHSHVENMLDLAWERGFVSQYECDEIRLPIFTPSQRARRLLDLATVKANGLAAFLLQHVQELPVPLALPL

SEQ ID NO: 283 - protein, 87 aa, Homo sapiens
QWIQSKREDIVNQMTEACLNQSLDALLSRDLIMKEDYELVSTKPTRTSKVRQLLDTTDIQGEEFAKVIVQKLKDNKQMGLQPYPEIL

SEQ ID NO: 284 - protein, 87 aa, Homo sapiens; identical to SEQ ID NO: 272

SEQ ID NO: 285 - protein, 94 aa, Homo sapiens
MEARDKQVLRSLRLELGAEVLVEGLVLQYLYQEGILTENHIQEIKAQTTGLRKTMLLLDILPSRGPKAFDTFLDSLQEFPWVREKLEKAREEVT

SEQ ID NO: 286 - protein, 90 aa, Homo sapiens
MDAKARNCLLQHREALEKDIKTSYIMDHMISDGFLTISEEEKVRNEPTQQQRAAMLIKMILKKDNDSYVSFYNALLHEGYKDLAALLHDG

SEQ ID NO: 287 - protein, 89 aa, Homo sapiens
AAKPGLHFIDQHRAALIARVTNVEWLLDALYGKVLTDEQYQAVRAEPTNPSKMRKLFSFTPAWNWTCKDLLLQALRESQSYLVEDLERS

SEQ ID NO: 288 - protein, 87 aa, Homo sapiens
MTTEQRRSLQAFQDYIRKTLDPTYILSYMAPWFREEEVQYIQAEKNNKGPMEAATLFLKFLLELQEEGWFRGFLDALDHAGYSGLYE

SEQ ID NO: 289 - protein, 81 aa, Homo sapiens
WDFKKIEKLEEYRLLLKRLQPEFKTRIIPTDIISDLSECLINQECEEILQICSTKGMMAGAEKLVECLLRSDKENWPKTLK

SEQ ID NO: 290 - protein, 320 aa, Homo sapiens
FLMNEVIKLQQQMKAKDLQRCELLARLRQLEDEKKQMTLTRVELLTFQERYYKMKEERDSYNDELVKVKDDNYNLAMRYAQLSEEKNMAVMRSRDLQLEIDQLKHRLNKMEEECKLERNQSLKLKNDIENRPKKEQVLELERENEMLKTKNQELQSIIQAGKRSLPDSDKAILDILEHDRKEALEDRQELVNRIYNLQEEARQAEELRDKYLEEKEDLELKCSTLGKDCEMYKHRMNTVMLQLEEVERERDQAFHSRDEAQTQYSQCLIEKDKYRKQIRELEEKNDEMRIEMVRREACIVNLESKLRRLSKDSNNLDQSL

SEQ ID NO: 291 - protein, 161 aa, Homo sapiens
QLLMTEVMKLQKKVQDLTALLSSKDDFIKELRVKDSLLRKHQERVQRLKEECEAGSRELKRCKEENYDLAMRLAHQSEEKGAALMRNRDLQLEIDQLKHSLMKAEDDCKVERKHTLKLRHAMEQRPSQELLWELQQEKALLQARVQELEASVQEGKLDRSS

SEQ ID NO: 292 - protein, 319 aa, Homo sapiens
TEVRRLREARKSQLQREQQLQARGRVLEEERAGLEQRLRDQQQAQERCQRLREDWEAGSLELLRLKDENYMIAMRLAQLSEEKNSAVLRSRDLQLAVDQLKLKVSRLEEECALLRRARGPPPGAEEKEKEKEKEKEPDNVDLVSELRAENQRLTASLRELQEGLQQEASRPGAPGSERILLDILEHDWREAQDSRQELCQKLHAVQGELQWAEELRDQYLQEMEDLRLKHRTLQKDCDLYKHRMATVLAQLEEIEKERDQAIQSRDRIQLQYSQSLIEKDQYRKQVRGLEAERDELLTTLTSLEGTKALLEVQLQRAQG

SEQ ID NO: 293 - protein, 282 aa, Homo sapiens
LAGAIGSLQEELNQEKGQKEVLLRRCQQLQEHLGLAETRAEGLHQLEADHSRMKREVSAHFHEVLRLKDEMLSLSLHYSNALQEKELAASRCRSLQEELYLLKQELQRANMVSSCELELQEQSLRTASDQESGDEELNRLKEENEKLRSLTFSLAEKDILEQSLDEARGSRQELVERIHSLRERAVAAERQREQYWEEKEQTLLQFQKSKMACQLYREKVNALQAQVCELQKERDQAYSARDSAQREISQSLVEKDSLRRQVFELTDQVCELRTQLRQLQAE

SEQ ID NO: 294 - protein, 135 aa, Homo sapiens
PRNLPVTIISQDFGDASPRTNGQEADDSSTSEESPEDSKYFLPYHPPQRRMNLKGIQLQRAKSPISLKRTSDFQAKGHEEEGTDASPSSCGSLPITNSFTKMQPPRSRSSIMSITAEPPGNDSIVRRYKEDAPHR

SEQ ID NO: 295 - protein, 217 aa, Homo sapiens
PRNLPVTIISQDFGDASPRTNGQEADDSSTSEESPEDSKYFLPYHPPQRRMNLKGIQLQRAKSPISLKRTSDFQAKGHEEEGTDASPSSCGSLPITNSFTKMQPPRSRSSIMSITAEPPGNDSIVRRYKEDAPHRSTVEEDNDSGGFDALDLDDDSHERYSFGPSSIHSSSSSHQSEGLDAYDLEQVNLMFRKFSLERPFRPSVTSVGHVRGPGPSV

SEQ ID NO: 296 - DNA, 507 bp, Synthetic construct
  source 1..507: mol_type = other DNA
gatgaacctgaagggcatccagctgcagagagccaaatcccccatcagcctgaagcgaacatcagattttcaagccaaggggcacgaggaagaaggcacggacgccagccctagctcctgcggatctctgcccatcaccaactccttcaccaagatgcagcccccccggagccgcagcagcatcatgtcaatcaccgccgagcccccgggaaacgactccatcgtcagacgctacaaggaggacgcgccccatcgcagatggctcaatcacaaaggagtgagacagaaacgcctgaatgtctggctgggaattaagaatgaggatgctgatgagaactattttatcaatgaggaagatgaaaacctgccccattatgatgagaaaacctggtttgttgaggatatcaatcgagtacaagcagaggacttgctttatgggaaacctgatggtgcattcttaattcgtgagagtagcaagaaaggatgctatgcttgctctgtggtggc

SEQ ID NO: 297 - protein, 168 aa, Synthetic construct
MNLKGIQLQRAKSPISLKRTSDFQAKGHEEEGTDASPSSCGSLPITNSFTKMQPPRSRSSIMSITAEPPGNDSIVRRYKEDAPHRRWLNHKGVRQKRLNVWLGIKNEDADENYFINEEDENLPHYDEKTWFVEDINRVQAEDLLYGKPDGAFLIRESSKKGCYACSVV

SEQ ID NO: 298 - protein, 1068 aa, Homo sapiens
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

SEQ ID NO: 299 - protein, 1070 aa, Homo sapiens
MCFSFIMPPAMADILDIWAVDSQIASDGSIPVDFLLPTGIYIQLEVPREATISYIKQMLWKQVHNYPMFNLLMDIDSYMFACVNQTAVYEELEDETRRLCDVRPFLPVLKLVTRSCDPGEKLDSKIGVLIGKGLHEFDSLKDPEVNEFRRKMRKFSEEKILSLVGLSWMDWLKQTYPPEHEPSIPENLEDKLYGGKLIVAVHFENCQDVFSFQVSPNMNPIKVNELAIQKRLTIHGKEDEVSPYDYVLQVSGRVEYVFGDHPLIQFQYIRNCVMNRALPHFILVECCKIKKMYEQEMIAIEAAINRNSSNLPLPLPPKKTRIISHVWENNNPFQIVLVKGNKLNTEETVKVHVRAGLFHGTELLCKTIVSSEVSGKNDHIWNEPLEFDINICDLPRMARLCFAVYAVLDKVKTKKSTKTINPSKYQTIRKAGKVHYPVAWVNTMVFDFKGQLRTGDIILHSWSSFPDELEEMLNPMGTVQTNPYTENATALHVKFPENKKQPYYYPPFDKIIEKAAEIASSDSANVSSRGGKKFLPVLKEILDRDPLSQLCENEMDLIWTLRQDCREIFPQSLPKLLLSIKWNKLEDVAQLQALLQIWPKLPPREALELLDFNYPDQYVREYAVGCLRQMSDEELSQYLLQLVQVLKYEPFLDCALSRFLLERALGNRRIGQFLFWHLRSEVHIPAVSVQFGVILEAYCRGSVGHMKVLSKQVEALNKLKTLNSLIKLNAVKLNRAKGKEAMHTCLKQSAYREALSDLQSPLNPCVILSELYVEKCKYMDSKMKPLWLVYNNKVFGEDSVGVIFKNGDDLRQDMLTLQMLRLMDLLWKEAGLDLRMLPYGCLATGDRSGLIEVVSTSETIADIQLNSSNVAAAAAFNKDALLNWLKEYNSGDDLDRAIEEFTLSCAGYCVASYVLGIGDRHSDNIMVKKTGQLFHIDFGHILGNFKSKFGIKRERVPFILTYDFIHVIQQGKTGNTEKFGRFRQCCEDAYLILRRHGNLFITLFALMLTAGLPELTSVKDIQYLKDSLALGKSEEEALKQFKQKFDEALRESWTTKVNWMAHTVRKDYRS

SEQ ID NO: 300 - protein, 1044 aa, Homo sapiens
MPPGVDCPMEFWTKEENQSVVVDFLLPTGVYLNFPVSRNANLSTIKQLLWHRAQYEPLFHMLSGPEAYVFTCINQTAEQQELEDEQRRLCDVQPFLPVLRLVAREGDRVKKLINSQISLLIGKGLHEFDSLCDPEVNDFRAKMCQFCEEAAARRQQLGWEAWLQYSFPLQLEPSAQTWGPGTLRLPNRALLVNVKFEGSEESFTFQVSTKDVPLALMACALRKKATVFRQPLVEQPEDYTLQVNGRHEYLYGSYPLCQFQYICSCLHSGLTPHLTMVHSSSILAMRDEQSNPAPQVQKPRAKPPPIPAKKPSSVSLWSLEQPFRIELIQGSKVNADERMKLVVQAGLFHGNEMLCKTVSSSEVSVCSEPVWKQRLEFDINICDLPRMARLCFALYAVIEKAKKARSTKKKSKKADCPIAWANLMLFDYKDQLKTGERCLYMWPSVPDEKGELLNPTGTVRSNPNTDSAAALLICLPEVAPHPVYYPALEKILELGRHSECVHVTEEEQLQLREILERRGSGELYEHEKDLVWKLRHEVQEHFPEALARLLLVTKWNKHEDVAQMLYLLCSWPELPVLSALELLDFSFPDCHVGSFAIKSLRKLTDDELFQYLLQLVQVLKYESYLDCELTKFLLDRALANRKIGHFLFWHLRSEMHVPSVALRFGLILEAYCRGSTHHMKVLMKQGEALSKLKALNDFVKLSSQKTPKPQTKELMHLCMRQEAYLEALSHLQSPLDPSTLLAEVCVEQCTFMDSKMKPLWIMYSNEEAGSGGSVGIIFKNGDDLRQDMLTLQMIQLMDVLWKQEGLDLRMTPYGCLPTGDRTGLIEVVLRSDTIANIQLNKSNMAATAAFNKDALLNWLKSKNPGEALDRAIEEFTLSCAGYCVATYVLGIGDRHSDNIMIRESGQLFHIDFGHFLGNFKTKFGINRERVPFILTYDFVHVIQQGKTNNSEKFERFRGYCERAYTILRRHGLLFLHLFALMRAAGLPELSCSKDIQYLKDSLALGKTEEEALKHFRVKFNEALRESWKTKVNWLAHNVSKDNRQ

SEQ ID NO: 301 - protein, 1367 aa, Homo sapiens
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTC

SEQ ID NO: 302 - protein, 1255 aa, Homo sapiens [sequence omitted]

SEQ ID NO: 303 - protein, 220 aa, Homo sapiens; identical to SEQ ID NO: 42

SEQ ID NO: 304 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 305 - protein, 95 aa, Homo sapiens
WFVEDINRVQAEDLLYGKPDGAFLIRESSKKGCYACSVVADGEVKHCVIYSTARGYGFAEPYNLYSSLKELVLHYQQTSLVQHNDSLNVRLAYPV

SEQ ID NO: 306 - protein, 13 aa, Homo sapiens
FDNLYYWDQDPPE

SEQ ID NO: 307 - protein, 95 aa, Homo sapiens; identical to SEQ ID NO: 305

SEQ ID NO: 308 - protein, 95 aa, Homo sapiens
WYVGKINRTQAEEMLSGKRDGTFLIRESSQRGCYACSVVVDGDTKHCVIYRTATGFGFAEPYNLYGSLKELVLHYQHASLVQHNDALTVTLAHPV

SEQ ID NO: 309 - protein, 95 aa, Homo sapiens
WNVGSSNRNKAENLLRGKRDGTFLVRESSKQGCYACSVVVDGEVKHCVINKTATGYGFAEPYNLYSSLKELVLHYQHTSLVQHNDSLNVTLAYPV

SEQ ID NO: 310 - protein, 103 aa, Homo sapiens
QPWGSILRNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTG

SEQ ID NO: 311 - protein, 98 aa, Homo sapiens
WFFKNLSRKDAERQLLAPGNTHGSFLIRESESTAGSFSLSVRDFDQNQGEVVKHYKIRNLDNGGFYISPRITFPGLHELVRHYTNASDGLCTRLSRPC

SEQ ID NO: 312 - protein, 97 aa, Homo sapiens
WFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPL

SEQ ID NO: 313 - protein, 101 aa, Synthetic construct
QAEEWYFGKLGRKDAERQLLSFGNPRGTFLIRESETVKGAYALSIRDWDDMKGDHVKHYLIRKLDNGGYYIWLGAQFETLQQLVQHYSERAVPGSCRLVVP

SEQ ID NO: 314 - protein, 101 aa, Synthetic construct
QAEEWYFGKLGRKDAERQLLSFGNPRGTFLIRESETVKGAYALSIRDWDDMKGDHVKHYLIRKLDNGGYYIPGGAQFETLQQLVQHYSERAWYWCCRLVVP

SEQ ID NO: 315 - protein, 101 aa, Synthetic construct
QAEEWYFGKLGRKDAERQLLSFGNPRGTFLIRESETVKGAYALSIRDWDDMKGDHVKHYLIRKLDNGGYYIRRRAQFETLQQLVQHYSERALPGCCRLVVP

SEQ ID NO: 316 - protein, 121 aa, Homo sapiens
KRSDRKDVQHNEWYIGEYSRQAVEEAFMKENKDGSFLVRDCSTKSKEEPYVLAVFYENKVYNVKIRFLERNQQFALGTGLRGDEKFDSVEDIIEHYKNFPIILIDGKDKTGVHRKQCHLTQ

SEQ ID NO: 317 - protein, 118 aa, Homo sapiens
FDSEERSSWYWGRLSRQEAVALLQGQRHGVFLVRDSSTSPGDYVLSVSENSRVSHYIINSSGPRPPVPPSPAQPPPGVSPSRLRIGDQEFDSLPALLEFYKIHYLDTTTLIEPVSRSR

SEQ ID NO: 318 - protein, 101 aa, Homo sapiens
FDSSDRSAWYMGPVSRQEAQTRLQGQRHGMFLVRDSSTCPGDYVLSVSENSRVSHYIINSLPNRRFKIGDQEFDHLPALLEFYKIHYLDTTTLIEPAPRYP

SEQ ID NO: 319 - protein, 95 aa, Homo sapiens
KPHPWYSGRISRQLAEEILMKRNHLGAFLIRESESSPGEFSVSVNYGDQVQHFKVLREASGKYFLWEEKFNSLNELVDFYRTTTIAKKRQIFLRD

SEQ ID NO: 320 - protein, 94 aa, Homo sapiens
QFPKWFHEGLSRHQAENLLMGKEVGFFIIRASQSSPGDFSISVRHEDDVQHFKVMRDNKGNYFLWTEKFPSLNKLVDYYRTNSISRQKQIFLRD

SEQ ID NO: 321 - protein, 95 aa, Homo sapiens
KPHPWFFGKIPRAKAEEMLSKQRHDGAFLIRESESAPGDFSLSVKFGNDVQHFKVLRDGAGKYFLWVVKFNSLNELVDYHRSTSVSRNQQIFLRD

SEQ ID NO: 322 - protein, 97 aa, Homo sapiens
NPWYYGKVTRHQAEMALNERGHEGDFLIRDSESSPNDFSVSLKAQGKNKHFKVQLKETVYCIGQRKFSTMEELVEHYKKAPIFTSEQGEKLYLVKHL

SEQ ID NO: 323 - protein, 98 aa, Homo sapiens
REWYYGNVTRHQAECALNERGVEGDFLIRDSESSPSDFSVSLKASGKNKHFKVQLVDNVYCIGQRRFHTMDELVEHYKKAPIFTSEHGEKLYLVRALQ

SEQ ID NO: 324 - protein, 98 aa, Homo sapiens
PGICVARVYHGWLFEGLGRDKAEELLQLPDTKVGSFMIRESETKKGFYSLSVRHRQVKHYRIFRLPNNWYYISPRLTFQCLEDLVNHYSEVADGLCCV

SEQ ID NO: 325 - protein, 104 aa, Homo sapiens
PSVHVAKVSHGWLYEGLSREKAEELLLLPGNPGGAFLIRESQTRRGSYSLSVRLSRPASWDRIRHYRIHCLDNGWLYISPRLTFPSLQALVDHYSELADDICCL

SEQ ID NO: 326 - protein, 225 aa, Homo sapiens
RVPKPKGVLPSHYYESFLEKKGPCDRDYKKFWAGLQGLTIYFYNSNRDFQHVEKLNLGAFEKLTDEIPWGSSRDPGTHFSLILRDQEIKFKVETLECREMWKGFILTVVELRVPTDLTLLPGHLYMMSPSCFLKVSRLEAQLLLERYPECGNLLLRPSGDGADGVSVTTRQMHNGTHVVRHYKVKREGPKYVIDVEQPFSCTSLDAVVNYFVSHTKKALVPFLLD

SEQ ID NO: 327 - protein, 205 aa, Homo sapiens
ADHKQEEDMKRKQQRTTYIKRVIAHPSFHNINFKQAEKMMETMDQGDVIIRPSSKGENHLTVTWKVSDGIYQHVDVREEGKENAFSLGATLWINSEEFEDLDEIVARYVQPMASFARDLLNHKYYQDCSGGDRKKLEELLIKTKKEKPTFIPYFICACKELPGKFLLGYQPRGKPRIEYVTVTPEGFRYRGQIFPTVNGLFRWFK

SEQ ID NO: 328 - protein, 159 aa, Homo sapiens
DLVYVTERIIAVSFPSTANEENFRSNLREVAQMLKSKHGGNYLLFNLSERRPDITKLHAKVLEFGWPDLHTPALEKICSICKAMDTWLNADPHNVVVLHNKGNRGRIGVVIAAYMHYSNISASADQALDRFAMKRFYEDKIVPIGQPSQRRYVHYFSGL

SEQ ID NO: 329 - protein, 116 aa, Homo sapiens
TSKYWYKPEISREQAIALLKDQEPGAFIIRDSHSFRGAYGLAMKVSSPPPTIMQQNKKGDMTHELVRHFLIETGPRGVKLKGCPNEPNFGSLSALVYQHSIIPLALPCKLVIPNRD

SEQ ID NO: 330 - protein, 114 aa, Homo sapiens
TSKFWYKPHLSRDQAIALLKDKDPGAFLIRDSHSFQGAYGLALKVATPPPSAQPWKGDPVEQLVRHFLIETGPKGVKIKGCPSEPYFGSLSALVSQHSISPISLPCCLRIPSKD

SEQ ID NO: 331 - protein, 117 aa, Homo sapiens
TSKFWYKADISREQAIAMLKDKEPGSFIVRDSHSFRGAYGLAMKVATPPPSVLQLNKKAGDLANELVRHFLIECTPKGVRLKGCSNEPYFGSLTALVCQHSITPLALPCKLLIPERD

SEQ ID NO: 332 - protein, 114 aa, Homo sapiens
TSKYWFKPNITREQAIELLRKEEPGAFVIRDSSSYRGSFGLALKVQEVPASAQSRPGEDSNDLIRHFLIESSAKGVHLKGADEEPYFGSLSAFVCQHSIMALALPCKLTIPQRE

SEQ ID NO: 333 - protein, 94 aa, Homo sapiens
EKHSWYHGPVSRNAAEYLLSSGINGSFLVRESESSPGQRSISLRYEGRVYHYRINTASDGKLYVSSESRFNTLAELVHHHSTVADGLITTLHYP

SEQ ID NO: 334 - protein, 94 aa, Homo sapiens
EKHSWYHGPVSRSAAEYLLSSLINGSFLVRESESSPGQLSISLRYEGRVYHYRINTTADGKVYVTAESRFSTLAELVHHHSTVADGLVTTLHYP

SEQ ID NO: 335 - protein, 100 aa, Homo sapiens
EMERWFFRSQGRKEAERQLLAPINKAGSFLIRESETNKGAFSLSVKDVTTQGELIKHYKIRCLDEGGYYISPRITFPSLQALVQHYSKKGDGLCQRLTLP

SEQ ID NO: 336 - protein, 106 aa, Homo sapiens
ENLDDYDWFAGNISRSQSEQLLRQKGKEGAFMVRNSSQVGMYTVSLFSKAVNDKKGTVKHYHVHTNAENKLYLAENYCFDSIPKLIHYHQHNSAGMITRLRHPVST

SEQ ID NO: 337 - protein, 106 aa, Homo sapiens
DSIEMYEWYSKHMTRSQAEQLLKQEGKEGGFIVRDSSKAGKYTVSVFAKSTGDPQGVIRHYVVCSTPQSQYYLAEKHLFSTIPELINYHQHNSAGLISRLKYPVSQ

SEQ ID NO: 338 - protein, 98 aa, Homo sapiens
SLMPWFHGKITREQAERLLYPPETGLFLVRESTNYPGDYTLCVSCDGKVEHYRIMYHASKLSIDEEVYFENLMQLVEHYTSDADGLCTRLIKPKVMEG

SEQ ID NO: 339 - protein, 86 aa, Homo sapiens
KPLAEQDWYHGAIPRIEAQELLKKQGDFLVRESHGKPGEYVLSVYSDGQRRHFIIQYVDNMYRFEGTGFSNIPQLIDHHYTTKQVI

SEQ ID NO: 340 - protein, 85 aa, Homo sapiens
KPLHEQLWYHGAIPRAEVAELLVHSGDFLVRESQGKQEYVLSVLWDGLPRHFIIQSLDNLYRLEGEGFPSIPLLIDHLLSTQQPL

SEQ ID NO: 341 - protein, 101 aa, Homo sapiens
QAEEWYFGKIGRKDAERQLLSPGNPQGAFLIRESETTKGAYSLSIRDWDQTRGDHVKHYKIRKLDMGGYYITTRVQFNSVQELVQHYMEVNDGLCNLLIAP

SEQ ID NO: 342 - protein, 96 aa, Homo sapiens
QAEPWFFGAIGRSDAEKQLLYSENKTGSFLIRESESQKGEFSLSVLDGAVVKHYRIKRLDEGGFFLTRRRIFSTLNEFVSHYTKTSDGLCVKLGKP

SEQ ID NO: 343 - protein, 101 aa, Homo sapiens
QAEEWYFGKLGRKDAERQLLSFGNPRGTFLIRESETTKGAYSLSIRDWDDMKGDHVKHYKIRKLDNGGYYITTRAQFETLQQLVQHYSERAAGLCCRLVVP

SEQ ID NO: 344 - protein, 104 aa, Homo sapiens
ETEEWFFKGISRKDAERQLLAPGNMLGSFMIRDSETTKGSYSLSVRDYDPRQGDTVKHYKIRTLDNGGFYISPRSTFSTLQELVDHYKKGNDGLCQKLSVPCMS

SEQ ID NO: 345 - protein, 109 aa, Homo sapiens
NNLETYEWYNKSISRDKAEKLLLDTGKEGAFMVRDSRTAGTYTVSVFTKAVVSENNPCIKHYHIKETNDNPKRYYVAEKYVFDSIPLLINYHQHNGGGLVTRLRYPVCF

SEQ ID NO: 346 - protein, 102 aa, Homo sapiens
DVAPPLIVHNIQNGCHGPICTEYAINKLRQEGSEEGMYVLRWSCTDFDNILMTVTCFEKSEQVQGAQKQFKNFQIEVQKGRYSLHGSDRSFPSLGDLMSHLK

SEQ ID NO: 347 - protein, 97 aa, Homo sapiens
EVAPPAVLENIQSNCHGPISMDFAISKLKKAGNQTGLYVLRCSPKDFNKYFLTFAVERENVIEYKHCLITKNENEEYNLSGTKKNFSSLKDLLNCYQ

SEQ ID NO: 348 - protein, 97 aa, Homo sapiens
EVAPPRLLEEVAEQCHGPITLDFAINKLKTGGSRPGSYVLRRSPQDFDSFLLTVCVQNPLGPDYKGCLIRRSPTGTFLLVGLSRPHSSLRELLATCW

SEQ ID NO: 349 - protein, 101 aa, Homo sapiens
EPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRP

SEQ ID NO: 350 - protein, 101 aa, Homo sapiens
ETEEWFFKDITRKDAERQLLAPGNSAGAFLIRESETLKGSFSLSVRDFDPVHGDVIKHYKIRSLDNGGYYISPRITFPCISDMIKHYQKQADGLCRRLEKA

SEQ ID NO: 351 - protein, 98 aa, Homo sapiens
SLMPWFHGKISGQEAVQQLQPPEDGLFLVRESARHPGDYVLCVSFGRDVIHYRVLHRDGHLTIDEAVFFCNLMDMVEHYSKDKGAICTKLVRPKRKHG

SEQ ID NO: 352 - protein, 128 aa, Homo sapiens
DNIEAVGKKLHEYNTQFQEKSREYDRLYEEYTRTSQEIQMKRTAIEAFNETIKIFEEQCQTQERYSKEYIEKFKREGNEKEIQRIMHNYDKLKSRISEIIDSRRRLEEDLKKQAAEYREIDKRMNSIK

SEQ ID NO: 353 - protein, 104 aa, Homo sapiens
PHHDEKTWNVGSSNRNKAENLLRGKRDGTFLVRESSKQGCYACSVVVDGEVKHCVINKTATGYGFAEPYNLYSSLKELVLHYQHTSLVQHNDSLNVTLAYPVYA

SEQ ID NO: 354 - protein, 111 aa, Homo sapiens
NMSLQDAEWYWGDISREEVNEKLRDTADGTFLVRDASTKMHGDYTLTLRKGGNNKLIKIFHRDGKYGFSDPLTFSSVVELINHYRNESLAQYNPKLDVKLLYPVSKYQQDQ

SEQ ID NO: 355 - protein, 100 aa, Homo sapiens
SEPWFFGCISRSEAVRRLQAEGNATGAFLIRVSEKPSADYVLSVRDTQAVRHYKIWRRAGGRLHLNEAVSFLSLPELVNYHRAQSLSHGLRLAAPCRKHE

SEQ ID NO: 356 - protein, 101 aa, Homo sapiens
QAEEWYFGKITRRESERLLLNAENPRGTFLVRESETTKGAYCLSVSDFDNAKGLNVKHYKIRKLDSGGFYITSRTQFNSLQQLVAYYSKHADGLCHRLTTV

SEQ ID NO: 357 - protein, 79 aa, Homo sapiens
PWYFSGVSRTQAQQLLLSPPNEPGAFLIRPSESSLGGYSLSVRAQAKVCHYRVSMAADGSLYLQKGRLFPGLEELLTYY

SEQ ID NO: 358 - protein, 203 aa, Homo sapiens
LPFFFGNITREEAEDYLVQGGMSDGLYLLRQSRNYLGGFALSVAHGRKAHHYTIERELNGTYAIAGGRTHASPADLCHYHSQESDGLVCLLKKPFNRPQGVQPKEKMPWFHGKISREESEQIVLIGSKTNGKFLIRARDNNGSYALCLLHEGKVLHYRIDKDKTGKLSIPEGKKFDTLWQLVEHYSYKADGLLRVLTVPCQKI

SEQ ID NO: 359 - protein, 99 aa, Homo sapiens
EKMPWFHGKISREESEQIVLIGSKTNGKFLIRARDNNGSYALCLLHEGKVLHYRIDKDKTGKLSIPEGKKFDTLWQLVEHYSYKADGLLRVLTVPCQKI

SEQ ID NO: 360 - protein, 107 aa, Homo sapiens
NNLDQYEWYCRNMNRSKAEQLLRSEDKEGGFMVRDSSQPGLYTVSLYTKFGGEGSSGFRHYHIKETTTSPKKYYLAEKHAFGSIPEIIEYHKHNAAGLVTRLRYPVS

SEQ ID NO: 361 - protein, 106 aa, Homo sapiens
TNLEIYEWYHRNITRNQAEHLLRQESKEGAFIVRDSRHLGSYTISVFMGARRSTEAAIKHYQIKKNDSGQWYVAERHAFQSIPELIWYHQHNAAGLMTRLRYPVGL

SEQ ID NO: 362 - protein, 98 aa, Homo sapiens
EVAPPRLVMSIRDGIHGPLLEPFVQAKLRPEDGLYLIHWSTSHPYRLILTVAQRSQAPDGMQSLRLRKFPIEQQDGAFVLEGWGRSFPSVRELGAALQ

SEQ ID NO: 363 - protein, 100 aa, Homo sapiens
QAEEWYFGKMGRKDAERLLLNPGNQRGIFLVRESETTKGAYSLSIRDWDEIRGDNVKHYKIRKLDNGGYYITTRAQFDTLQKLVKHYTEHADGLCHKLTT

SEQ ID NO: 364 - protein, 104 aa, Homo sapiens
LIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACP

SEQ ID NO: 365 - protein, 104 aa, Homo sapiens
LPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQ

SEQ ID NO: 366 - protein, 99 aa, Homo sapiens
RWFHPNITGVEAENLLLTRGVDGSFLARPSKSNPGDFTLSVRRNGAVTHIKIQNTGDYYDLYGGEKFATLAELVQYYMEHHGQLKEKNGDVIELKYPLN

SEQ ID NO: 367 - protein, 108 aa, Homo sapiens
RWFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPLNT

SEQ ID NO: 368 - protein, 99 aa, Homo sapiens
RWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLN

SEQ ID NO: 369 - protein, 107 aa, Homo sapiens
RWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYA

SEQ ID NO: 370 - protein, 102 aa, Homo sapiens
MVPCWNHGNITRSKAEELLSRTGKDGSFLVRASESISRAYALCVLYRNCVYTYRILPNEDDKFTVQASEGVSMRFFTKLDQLIEFYKKENMGLVTHLQYPVP

SEQ ID NO: 371 - protein, 103 aa, Homo sapiens
QAPSWYHRDLSRAAAEELLARAGRDGSFLVRDSESVAGAFALCVLYQKHVHTYRILPDGEDFLAVQTSQGVPVRRFQTLGELIGLYAQPNQGLVCALLLPVEG

SEQ ID NO: 372 - protein, 111 aa, Homo sapiens
PPSLQDAEWYWGDISREEVNEKLRDTPDGTFLVRDASSKIQGEYTLTLRKGGNNKLIKVFHRDGHYGFSEPLTFCSVVDLINHYRHESLAQYNAKLDTRLLYPVSKYQQDQ

SEQ ID NO: 373 - protein, 103 aa, Homo sapiens
PHHEERTWYVGKINRTQAEEMLSGKRDGTFLIRESSQRGCYACSVVVDGDTKHCVIYRTATGFGFAEPYNLYGSLKELVLHYQHASLVQHNDALTVTLAHPVR

SEQ ID NO: 374 - protein, 128 aa, Homo sapiens
DNIDAVGKKLQEYHSQYQEKSKEYDRLYEEYTRTSQEIQMKRTAIEAFNETIKIFEEQCHTQEQHSKEYIERFRREGNEKEIERIMMNYDKLKSRLGEIHDSKMRLEQDLKNQALDNREIDKKMNSIK

SEQ ID NO: 375 - protein, 109 aa, Homo sapiens
SLQDAEWYWGDISREEVNDKLRDMPDGTFLVRDASTKMQGDYTLTLRKGGNNKLIKIYHRDGKYGFSDPLTFNSVVELINHYHHESLAQYNPKLDVKLMYPVSRYQQDQ

SEQ ID NO: 376 - protein, 104 aa, Homo sapiens
PHYDEKTWFVEDINRVQAEDLLYGKPDGAFLIRESSKKGCYACSVVADGEVKHCVIYSTARGYGFAEPYNLYSSLKELVLHYQQTSLVQHNDSLNVRLAYPVHA

SEQ ID NO: 377 - protein, 103 aa, Homo sapiens
HESKEWYHASLTRAQAEHMLMRVPRDGAFLVRKRNEPNSYAISFRAEGKIKHCRVQQEGQTVMLGNSEFDSLVDLISYYEKHPLYRKMKLRYPINEEALEKIG

SEQ ID NO: 378 - protein, 105 aa, Homo sapiens
HSNEKWFHGKLGAGRDGRHIAERLLTEYCIETGAPDGSFLVRESETFVGDYTLSFWRNGKVQHCRIHSRQDAGTPKFFLTDNLVFDSLYDLITHYQQVPLRCNEF

SEQ ID NO: 379 - protein, 104 aa, Homo sapiens
HESKPWYYDSLSRGEAEDMLMRIPRDGAFLIRKREGSDSYAITFRARGKVKHCRINRDGRHFVLGTSAYFESLVELVSYYEKHSLYRKMRLRYPVTPELLERYN

SEQ ID NO: 380 - protein, 101 aa, Homo sapiens
HFGEKWFHKKVEKRTSAEKLLQEYCMETGGKDGTFLVRESETFPNDYTLSFWRSGRVQHCRIRSTMEGGTLKYYLTDNLTFSSIYALIQHYRETHLRCAEF

SEQ ID NO: 381 - protein, 84 aa, Homo sapiens
WYAGACDRKSAEEALHRSNKDGSFLIRKSSGHDSKQPYTLVVFFNKRVYNIPVRFIEATKQYALGRKKNGEEYFGSVAEIIRNH

SEQ ID NO: 382 - protein, 121 aa, Homo sapiens
PAEEENSLNEEWYVSYITRPEAEAALRKINQDGTFLVRDSSKKTTTNPYVLMVLYKDKVYNIQIRYQKESQVYLLGTGLRGKEDFLSVSDIIDYFRKMPLLLIDGKNRGSRYQCTLTHAAG

SEQ ID NO: 383 - protein, 77 aa, Homo sapiens
WAFAGISRPCALALLRRDVLGAFLLWPELGASGQWCLSVRTQCGVVPHQVFRNHLGRYCLEHLPAEFPSLEALVENH

SEQ ID NO: 384 - protein, 104 aa, Homo sapiens
AEQLRGEPWFHGKLSRREAEALLQLNGDFLVRESTTTPGQYVLTGLQSGQPKHLLLVDPEGVVRTKDHRFESVSHLISYHMDNHLPIISAGSELCLQQPVERKL

SEQ ID NO: 385 - protein, 104 aa, Homo sapiens
EEQLRQEPWYHGRMSRRAAERMLRADGDFLVRDSVTNPGQYVLTGMHAGQPKHLLLVDPEGVVRTKDVLFESISHLIDHHLQNGQPIVAAESELHLRGVVSREP

SEQ ID NO: 386 - protein, 103 aa, Homo sapiens
EELQAETWYQGEMSRKEAEGLLEKDGDFLVRKSTTNPGSFVLTGMHNGQAKHLLLVDPEGTIRTKDRVFDSISHLINHHLESSLPIVSAGSELCLQQPVERKQ

SEQ ID NO: 387 - protein, 101 aa, Homo sapiens
KQQLWSEECYHGKLSRKAAESLLVKDGDFLVRESATSPGQYVLSGLQGGQAKHLLLVDPEGKVRTKDHVFDNVGHLIRYHMDNSLPIISSGSEVSLKQPVR

SEQ ID NO: 388 - protein, 85 aa, Homo sapiens
WYSGNCDRYAVESALLHLQKDGAYTVRPSSGPHGSQPFTLAVLLRGRVFNIPIRRLDGGRHYALGREGRNREELFSSVAAMVQHF

SEQ ID NO: 389 - protein, 88 aa, Homo sapiens
LRESGWYWGSITASEARQHLQKMPEGTFLVRDSTHPSYLFTLSVKTTRGPTNVRIEYADSSFRLDSNCLSRPRILAFPDVVSLVQHYV

SEQ ID NO: 390 - protein, 92 aa, Homo sapiens
ELLQDLGWYHGNLTRHAAEALLLSNGCDGSYLLRDSNETTGLYSLSVRAKDSVKHFHVEYTGYSFKFGFNEFSSLKDFVKHFANQPLIGSET

SEQ ID NO: 391 - protein, 108 aa, Homo sapiens
IHRTQHWFHGRISREESHRIIKQQGLVDGLFLLRDSQSNPKAFVLTLCHHQKIKNFQILPCEDDGQTFFSLDDGNTKFSDLIQLVDFYQLNKGVLPCKLKHHCIRVAL

SEQ ID NO: 392 - protein, 108 aa, Homo sapiens
IHRSQPWFHHKISRDEAQRLIIQQGLVDGVFLVRDSQSNPKTFVLSMSHGQKIKHFQIIPVEDDGEMFHTLDDGHTRFTDLIQLVEFYQLNKGVLPCKLKHYCARIAL

SEQ ID NO: 393 - protein, 108 aa, Homo sapiens
IHRTQLWFHGRISREESQRLIGQQGLVDGLFLVRESQRNPQGFVLSLCHLQKVKHYLILPSEEEGRLYFSMDDGQTRFTDLLQLVEFHQLNRGILPCLLRHCCTRVAL

SEQ ID NO: 394 - protein, 102 aa, Homo sapiens
LAQDGVPEWFHGAISREDAENLLESQPLGSFLIRVSHSHVGYTLSYKAQSSCCHFMVKLLDDGTFMIPGEKVAHTSLDALVTFHQQKPIEPRRELLTQPCRQ

SEQ ID NO: 395 - protein, 97 aa, Homo sapiens
GDQPLSGYPWFHGMLSRLKAAQLVLTGGTGSHGVFLVRQSETRRGEYVLTFNFQGKAKHLRLSLNEEGQCRVQHLWFQSIFDMLEHFRVHPIPLESG

SEQ ID NO: 396 - protein, 96 aa, Homo sapiens
ELELSDYPWFHGTLSRVKAAQLVLAGGPRNHGLFVIRQSETRPGEYVLTFNFQGKAKHLRLSLNGHGQCHVQHLWFQSVLDMLRHFHTHPIPLESG

SEQ ID NO: 397 - protein, 97 aa, Homo sapiens
TDHFLSCYPWFHGPISRVKAAQLVQLQGPDAHGVFLVRQSETRRGEYVLTFNFQGIAKHLRLSLTERGQCRVQHLHFPSVVDMLHHFQRSPIPLECG

SEQ ID NO: 398 - protein, 103 aa, Homo sapiens
DAVAVYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEK

SEQ ID NO: 399 - protein, 103 aa, Homo sapiens
MDLPYYHGRLTKQDCETLLLKEGVDGNFLLRDSESIPGVLCLCVSFKNIVYTYRIFREKHGYYRIQTAEGSPKQVFPSLKELISKFEKPNQGMVVHLLKPIKR

SEQ ID NO: 400 - protein, 102 aa, Homo sapiens
LQHGAAPAWFHGFITRREAERLLEPKPQGCYLVRFSESAVTFVLTYRSRTCCRHFLLAQLRDGRHVVLGEDSAHARLQDLLLHYTAHPLSPYGETLTEPLAR

SEQ ID NO: 401 - protein, 104 aa, Homo sapiens
KTSDTIAPWFHGILTLKKANELLLSTGMPGSFLIRVSERIKGYALSYLSEDGCKHFLIDASADAYSFLGVDQLQHATLADLVEYHKEEPITSLGKELLLYPCGQ

SEQ ID NO: 402 - protein, 103 aa, Homo sapiens
RNTKFIAPWFHGIISREDAEALLENMTEGAFLVRVSEKIWGYTLSYRLQKGFKHFLVDASGDFYSFLGVDPNRHATLTDLVDFHKEEIITVSGGELLQEPCGQ

SEQ ID NO: 403 - protein, 101 aa, Homo sapiens
SVFVNTTESCEVERLFKATSPRGEPQDGLYCIRNSSTKSGKVLVVWDETSNKVRNYRIFEKDSKFYLEGEVLFVSVGSMVEHYHTHVLPSHQSLLLRHPYG

SEQ ID NO: 404 - protein, 97 aa, Homo sapiens
QIWYHGAISRGDAENLLRLCKECSYLVRNSQTSKHDYSLSLRSNQGFMHMKLAKTKEKYVLGQNSPPFDSVPEVIHYYTTRKLPIKGAEHLSLLYPV

SEQ ID NO: 405 - protein, 98 aa, Homo sapiens
QPWFHGPLNRADAESLLSLCKEGSYLVRLSETNPQDCSLSLRSSQGFLHLKFARTRENQVVLGQHSGPFPSVPELVLHYSSRPLPVQGAEHLALLYPV

SEQ ID NO: 406 - protein, 73 aa, Homo sapiens
QPWYHGAISRAEAESRLQPCKEAGYLVRNSESGNSRYSIALKTSQGCVHIIVAQTKDNKYTLNQTSAVFDSIP

SEQ ID NO: 407 - protein, 98 aa, Homo sapiens
QVWYHGAISRTDAENLLRLCKEASYLVRNSETSKNDFSLSLKSSQGFMHMKLSRTKEHKYVLGQNSPPFSSVPEIVHHYASRKLPIKGAEHMSLLYPV

SEQ ID NO: 408 - protein, 98 aa, Homo sapiens
TRASALLDACGFYWGPLSVHGAHERLRAEPVGTFLVRDSRQRNCFFALSVKMASGPTSIRVHFQAGRFHLDGSRESFDCLFELLEHYVAAPRRMLGAP

SEQ ID NO: 409 - protein, 103 aa, Homo sapiens
LRELGQTGWYWGSMTVNEAKEKLKEAPEGTFLIRDSSHSDYLLTISVKTSAGPTNLRIEYQDGKFRLDSIICVKSKLKQFDSVVHLIDYYVQMCKDKRTGPEA

SEQ ID NO: 410 - protein, 101 aa, Homo sapiens
VNAVRKLQESGFYWSAVTGGEANLLLSAEPAGTFLIRDSSDQRHFFTLSVKTQSGTKNLRIQCEGGSFSLQSDPRSTQPVPRFDCVLKLVHHYMPPPGAPS

SEQ ID NO: 411 - protein, 101 aa, Homo sapiens
VPDLLQINNNPCYWGVMDKYAAEALLEGKPEGTFLLRDSAQEDYLFSVSFRRYSRSLHARIEQWNHNFSFDAHDPCVFHSPDITGLLEHYKDPSACMFFEP

SEQ ID NO: 412 - protein, 101 aa, Homo sapiens
VPDLLQITGNPCYWGVMDRYEAEALLEGKPEGTFLLRDSAQEDYLFSVSFRRYNRSLHARIEQWNHNFSFDAHDPCVFHSSTVTGLLEHYKDPSSCMFFEP

SEQ ID NO: 413 - protein, 100 aa, Homo sapiens
TEELKKLAKQGWYWGPITRWEAEGKLANVPDGSFLVRDSSDDRYLLSLSFRSHGKTLHTRIEHSNGRFSFYEQPDVEGHTSIVDLIEHSIRDSENGAFCY

SEQ ID NO: 414 - protein, 101 aa, Homo sapiens
AASLRELEKCGWYWGPMNWEDAEMKLKGKPDGSFLVRDSSDPRYILSLSFRSQGITHHTRMEHYRGTFSLWCHPKFEDRCQSVVEFIKRAIMHSKNGKFLY

SEQ ID NO: 415 - protein, 136 aa, Homo sapiens
EDLRSHAWYHGRIPRQVSENLVQRDGDFLVRDSLSSPGNFVLTCQWKNLAQHFKINRTVLRLSEAYSRVQYQFEMESFDSIPGLVRCYVGNRRPISQQSGAIIFQPINRTVPLRCLEEHYGTSPGQAREGSLTKGR

SEQ ID NO: 416 - protein, 87 aa, Homo sapiens
RPKYYGREFHGMISREAADQLLIVAEGSYLIRESQRQPGTYTLALRFGSQTRNFRLYYDGKHFVGEKRFESIHDLVTDGLITLYIET

SEQ ID NO: 417 - protein, 87 aa, Homo sapiens
RPKYYGREFHGIISREQADELLGGVEGAYILRESQRQPGCYTLALRFGNQTLNYRLFHDGKHFVGEKRFESIHDLVTDGLITLYIET

SEQ ID NO: 418 - protein, 181 aa, Homo sapiens
GPEYEEEEVAIPLTAPPTNQWYHGKLDRTIAEERLRQAGKSGSYLIRESDRRPGSFVLSFLSQMNVVNHFRIIAMCGDYYIGGRRFSSLSDLIGYYSHVSCLLKIWFHGKISKQEAYNLLMTVGQVCSFLVRPSDNTPGDYSLYFRTNENIQRFKICPTPNNQFMMGGRYYNSIGDIIDHY

SEQ ID NO: 419 - protein, 101 aa, Homo sapiens
LRERLLLTRPVWLQLQANAAAALHMLRTEPPGTFLVRKSNTRQCQALCMRLPEASGPSFVSSHYILESPGGVSLEGSELMFPDLVQLICAYCHTRDILLLP

SEQ ID NO: 420 - protein, 100 aa, Homo sapiens
ILDRLLHTHPIWLQLSLSEEEAAEVLQAQPPGIFLVHKSTKMQKKVLSLRLPCEFGAPLKEFAIKESTYTFSLEGSGISFADLFRLIAFYCISRDVLPFT

SEQ ID NO: 421 - protein, 102 aa, Homo sapiens
ILEKLIKTCPVWLQLSLGQAEVARILHRVVAGMFLVRRDSSSKQLVLCVHFPSLNESSAEVLEYTIKEEKSILYLEGSALVFEDIFRLIAFYCVSRDLLPFT

SEQ ID NO: 422 - protein, 136 aa, Homo sapiens
EDLAGQPWYHGLLSRQKAEALLQQNGDFLVRASGSRGGNPVISCRWRGSALHFEVFRVALRPRPGRPTALFQLEDEQFPSIPALVHSYMTGRRPLSQATGAVVSRPVTWQGPLRRSFSEDTLMDGPARIEPLRARK

SEQ ID NO: 423 - protein, 135 aa, Homo sapiens
TDLRSHAWYHGRIPREVSETLVQRNGDFLIRDSLTSLGDYVLTCRWRNQALHFKINKVVVKAGESYTHIQYLFEQESFDHVPALVRYHVGSRKAVSEQSGAIIYCPVNRTFPLRYLEASYGLGQGSSKPASPVSP

SEQ ID NO: 424 - protein, 103 aa, Homo sapiens
DLSVHLWYAGPMERAGAESILANRSDGTFLVRQRVKDAAEFAISIKYNVEVKHIKIMTAEGLYRITEKKAFRGLTELVEFYQQNSLKDCFKSLDTTLQFPFKE

SEQ ID NO: 425 - protein, 103 aa, Homo sapiens
DYTAYPWFAGNMERQQTDNLLKSHASGTYLIRERPAEAERFAISIKFNDEVKHIKVVEKDNWIHITEAKKFDSLLELVEYYQCHSLKESFKQLDTTLKYPYKS

SEQ ID NO: 426 - protein, 103 aa, Homo sapiens
DYSCQPWYAGAMERLQAETELINRVNSTYLVRHRTKESGEYAISIKYNNEAKHIKILTRDGFFHIAENRKFKSLMELVEYYKHHSLKEGFRTLDTTLQFPYKE

SEQ ID NO: 427 - protein, 93 aa, Homo sapiens
PACFYTVSRKEATEMLQKNPSLGNMILRPGSDSRNYSITIRQEIDIPRIKHYKVMSVGQNYTIELEKPVTLPNLFSVIDYFVKETRGNLRPFI

SEQ ID NO: 428 - protein, 151 aa, Homo sapiens
WIESILELIKKHLLPLWNDGCIMGFISKERERALLKDQQPGTFLLRFSESSREGAITFTWVERSQNGGEPDFHAVEPYTKKELSAVTFPDIIRNYKVMAAENIPENPLKYLYPNIDKDHAFGKYYSRPKEAPEPMELDGPKGTGYIKTELI

SEQ ID NO: 429 - protein, 151 aa, Homo sapiens
WLDKILELVHDHLKDLWNDGRIMGFVSRSQERRLLKKTMSGTFLLRFSESSEGGITCSWVEHQDDDKVLIYSVQPYTKEVLQSLPLTEIIRHYQLLTEENIPENPLRFLYPRIPRDEAFGCYYQEKVNLQERRKYLKHRLIVVSNRQVDEL

SEQ ID NO: 430 - protein, 162 aa, Homo sapiens
MAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTP

SEQ ID NO: 431 - protein, 148 aa, Homo sapiens
WLEAILDLIKKHILPLWIDGYVMGFVSKEKERLLLKDKMPGTFLLRFSESHLGGITFTWVDHSESGEVRFHSVEPYNKGRLSALPFADILRDYKVIMAENIPENPLKYLYPDIPKDKAFGKHYSSQPCEVSRPTERGDKGYVPSVFIP

SEQ ID NO: 432 - protein, 140 aa, Homo sapiens
WFDGVMEVLKKHHKPHWNDGAILGFVNKQQAHDLLINKPDGTFLLRFSDSEIGGITIAWKFDSPERNLWNLKPFTTRDFSIRSLADRLGDLSYLIYVFPDRPKDEVFSKYYTPVLAKAVDGYVKPQIKQVVPEFVNASAD

SEQ ID NO: 433 - protein, 145 aa, Homo sapiens
WFDGVMEVLKKHLKPHWNDGAILGFVNKQQAHDLLINKPDGTFLLRFSDSEIGGITIAWKFDSQERMFWNLMPFTTRDFSIRSLADRLGDLNYLIYVFPDRPKDEVYSKYYTPVPCESATAKAVDGYVKPQIKQVVPEFVNASAD

SEQ ID NO: 434 - protein, 130 aa, Homo sapiens
WFDGVLDLTKRCLRSYWSDRLIIGFISKQYVTSLLLNEPDGTFLLRFSDSEIGGITIAHVIRGQDGSPQIENIQPFSAKDLSIRSLGDRIRDLAQLKNLYPKKPKDEAFRSHYKPEQMGKDGRGYVPATI

SEQ ID NO: 435 - protein, 97 aa, Homo sapiens
RNWNSLAVTHPGYMAFLTYDEVKARLQKFIHKPGSYIFRLSCTRLGQWAIGYVTADGNILQTIPHNKPLFQALIDGFREGFYLFPDGRNQNPDLTGL

SEQ ID NO: 436 - protein, 97 aa, Homo sapiens
RNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTGL

SEQ ID NO: 437 - protein, 97 aa, Homo sapiens
KNWQLLAVNHPGYMAFLTYDEVQERLQACRDKPGSYIFRPSCTRLGQWAIGYVSSDGSILQTIPANKPLSQVLLEGQKDGFYLYPDGKTHNPDLTEL